(19) United States
(12) Patent Application Publication
(10) Pub. No.: US 2020/0071382 A1
(43) Pub. Date: Mar. 5, 2020

(54) TGF-BETA SUPERFAMILY HETEROMULTIMERS AND USES THEREOF

(71) Applicant: Acceleron Pharma Inc., Cambridge, MA (US)

(72) Inventors: Ravindra Kumar, Acton, MA (US); Asya Grinberg, Lexington, MA (US); Dianne Sako, Medford, MA (US); Roselyne Castonguay, Watertown, MA (US)

(73) Assignee: Acceleron Pharma Inc., Cambridge, MA (US)

(21) Appl. No.: 16/315,338

(22) PCT Filed: Jul. 6, 2017
(86) PCT No.: PCT/US2017/040849

§ 371 Date: Jan. 4, 2019

Related U.S. Application Data
(60) Provisional application No. 62/359,614, filed on Jul. 7, 2016.
(60) Provisional application No. 62/404,670, filed on Oct. 5, 2016.

(51) Int. Cl. C07K 14/71; C07K 14/705; C07K 14/47; C12N 15/85

(52) U.S. Cl. CPC C07K 14/71; C07K 14/70596; A61K 38/00; C12N 15/85; C07K 2319/30; C07K 14/47

(57) ABSTRACT

The present invention discloses heterodimers comprising endoglin-Fc and ALKI-Fc, or hetero dimers comprising endoglin-Fc and ALK2-Fc. The Fe domains can include amino acid mutations that promote heterodimer formation. In certain aspects, the disclosure provides heteromeric polypeptide complexes comprising a co-receptor of the TGF-beta superfamily and an extracellular domain of a type I serine/threonine kinase receptor of the TGF beta superfamily, an extracellular domain of a type II serine/threonine kinase receptor of the TGF-beta superfamily, or an additional co-receptor of the TGF-beta superfamily. In some embodiments, the disclosure provides heteromultimers comprising a ligand-domain of one or more co-receptor selected from: endoglin, Cripto-1, Cryptic, Cryptic family protein IB, CRIMI, CRIM2, BAMBI, BMPER, RGM-A, RGMB, hemojuvelin, betaglycan, and MuSK. In some embodiments, the disclosure provides soluble heteromultimers comprising a ligand-domain of a co-receptor and a ligand-binding domain of a type II receptor selected from: ActRIIA, ActRIIB. TGFBRII, BMPRII, and MISRII. In some embodiments, the disclosure provides soluble heteromultimers comprising a ligand-domain of a co-receptor and a ligand-binding domain of a type I receptor selected from: ALK1, ALK2, ALK3, ALK4, ALK5, ALK6, and ALK7. In certain aspects, such TGF-beta superfamily heteromultimers may be used to regulate (promote or inhibit) growth of tissues or cells including, for example, bone and hematopoietic lineages, including red blood cells.

### CROSS-REFERENCE TO RELATED APPLICATIONS

This application is a national stage filing under 35 U.S.C. § 371 of International Application No. PCT/US2017/040849, filed on Jul. 6, 2017, which claims the benefit of priority to U.S. provisional application Ser. No. 62/359,614, filed on Jul. 7, 2016 and 62/404,670, filed on Oct. 5, 2016. The disclosures of each of the foregoing applications are hereby incorporated by reference in their entirety.

### SEQUENCE LISTING

The instant application contains a Sequence Listing which has been submitted via EFS-Web and is hereby incorporated by reference in its entirety. Said ASCII copy, created on Jan. 4, 2019, is named 1848493-0002-113-301_Seq.txt and is 668,081 bytes in size.

### BACKGROUND OF THE INVENTION

The transforming growth factor-beta (TGF-beta) superfamily contains a variety of growth factors that share common sequence elements and structural motifs. These proteins are known to exert biological effects on a large variety of cell types in both vertebrates and invertebrates. Members of the superfamily perform important functions during embryonic development in pattern formation and tissue specification and can influence a variety of differentiation processes, including adipogenesis, myogenesis, chondrogenesis, cardiogenesis, hematopoiesis, neurogenesis, and epithelial cell differentiation. The family is divided into two general phylogenetic clades: the more recently evolved members of the superfamily, which includes TGF-betas, Activins, and nodal and the Glade of more distantly related proteins of the superfamily, which includes a number of BMPs and GDFs. Hinck (2012) FEBS Letters 586:1860-1870. TGF-beta family members have diverse, often complementary biological effects. By manipulating the activity of a member of the TGF-beta family, it is often possible to cause significant physiological changes in an organism. For example, the Piedmontese and Belgian Blue cattle breeds carry a loss-of-function mutation in the GDF8 (also called myostatin) gene that causes a marked increase in muscle mass. Grobet et al. (1997) Nat Genet., 17(1):71-4. Furthermore, in humans, inactive alleles of GDF8 are associated with increased muscle mass and, reportedly, exceptional strength. Schuelke et al. (2004) N Engl J Med, 350:2682-8.

Changes in bone, red blood cells, and other tissues may be achieved by enhancing or inhibiting signaling (e.g., SMAD 1, 2, 3, 5, and/or 8) that is mediated by ligands of the TGF-beta family. Thus, there is a need for agents that regulate the activity of various ligands of the TGF-beta superfamily.

### SUMMARY OF THE INVENTION

In part, the disclosure provides recombinant TGF-beta superfamily heteromultimers (heteromultimers) comprising at least one TGF-beta superfamily co-receptor polypeptide (e.g., endoglin, betaglycan, Cripto-1, Cryptic, Cryptic family protein 1B, Crim1, Crim2, BAMBI, BMPER, RGM-A, RGM-B, MuSK, and hemojuvelin), including fragments and variants thereof. In some embodiments, the disclosure relates to a recombinant heteromultimer comprising a TGF-beta superfamily co-receptor polypeptide selected from the group consisting of: endoglin, betaglycan, Cripto-1, Cryptic, Cryptic family protein 1B, Crim1, Crim2, BAMBI, BMPER, RGM-A, RGM-B, MuSK, and hemojuvelin, including fragments and variants thereof, and a TGF-beta superfamily type I receptor polypeptide selected from the group consisting of: ALK1, ALK2, ALK3, ALK4, ALK5, ALK6, and ALK7, including fragments and variants thereof. In some embodiments, the disclosure relates to a recombinant heteromultimer comprising a TGF-beta superfamily co-receptor polypeptide selected from the group consisting of: endoglin, betaglycan, Cripto-1, Cryptic, Cryptic family protein 1B, Crim1, Crim2, BAMBI, BMPER, RGM-A, RGM-B, MuSK, and hemojuvelin, including fragments and variants thereof, and a TGF-beta superfamily type II receptor polypeptide selected from the group consisting of: ActRIIA, ActRIIB, TGFBRII, BMPRII, and MISRII, including fragments and variants thereof. In some embodiments, the disclosure relates to a recombinant heteromultimer comprising a first TGF-beta superfamily co-receptor polypeptide selected from the group consisting of: endoglin, betaglycan, Cripto-1, Cryptic, Cryptic family protein 1B, Crim1, Crim2, BAMBI, BMPER, RGM-A, RGM-B, MuSK, and hemojuvelin, including fragments and variants thereof, and a second TGF-beta superfamily co-receptor polypeptide selected from the group consisting of: endoglin, betaglycan, Cripto-1, Cryptic, Cryptic family protein 1B, Crim1, Crim2, BAMBI, BMPER, RGM-A, RGM-B, MuSK, and hemojuvelin, including fragments and variants thereof. Preferably, TGF-beta superfamily co-receptor, type I receptor, and type II receptor polypeptides as described herein comprise a ligand-binding domain of the receptor, for example, an extracellular domain of a TGF-beta superfamily co-receptor, type I receptor, or type II receptor. In other preferred embodiments, polypeptides and heteromultimers of the disclosure (e.g., co-receptor:type I receptor, co-receptor:type II receptor, and co-receptor:co-receptor heteromultimers) are soluble. In certain preferred embodiments, heteromultimers of the disclosure (e.g., co-receptor:type I receptor, co-receptor:type II receptor, and co-receptor:co-receptor heteromultimers) bind to one or more TGF-beta superfamily ligands (e.g., BMP2, BMP2/7, BMP3, BMP4, BMP4/7, BMP5, BMP6, BMP7, BMP8a, BMP8b, BMP9, BMP10, GDF3, GDF5, GDF6/BMP13, GDF7, GDF8, GDF9b/BMP15, GDF11/BMP11, GDF15/MIC1, TGF-β1, TGF-β2, TGF-β3, activin A, activin B, activin C, activin E, activin AB, activin AC, activin AE, activin BC, activin BE, nodal, glial cell-derived neurotrophic factor (GDNF), neurturin, artemin, persephin, Müllerian-inhibiting substance (MIS), and Lefty). In some embodiments, a heteromultimer (e.g., co-receptor:type I receptor, co-receptor:type II receptor, and co-receptor:co-receptor heteromultimers) may bind to one or more TGF-beta superfamily ligands with a K_{D} of at least 1×10⁻⁷ M (e.g., K_{D} of greater than or equal to 10⁻⁷, 10⁻⁸, 10⁻⁹, 10⁻¹⁰, 10⁻¹¹, or 10⁻¹²). In some embodiments, a heteromultimer of the disclosure (e.g., co-receptor:type I receptor, co-receptor:type II receptor, and co-receptor:co-receptor heteromultimers) has a different TGF-beta superfamily ligand binding and/or inhibition profile (specificity) compared to a corresponding homomultimer (e.g., endoglin:ALK1 heteromultimer vs. endoglin and ALK1 homomultimers). In some embodiments, a heteromultimer of the disclosure (e.g., co-receptor:type I receptor, co-receptor:type II receptor, and co-receptor:co-receptor heteromultimers) may inhibit one or more TGF-beta superfamily ligands (e.g., BMP2, BMP2/7, BMP3, BMP4, BMP4/7, BMP5, BMP6, BMP7, BMP8a, BMP8b, BMP9, BMP10, GDF3, GDF5, GDF6/BMP13, GDF7, GDF8, GDF9b/BMP15, GDF11/BMP11, GDF15/MIC1, TGF-β1, TGF-β2, TGF-β3, activin A, activin B, activin C, activin E, activin AB, activin AC, activin AE, activin BC, activin BE, nodal, glial cell-derived neurotrophic factor (GDNF), neurturin, artemin, persephin, Müllerian-inhibiting substance (MIS), and Lefty). In some embodiments, a heteromultimer of the disclosure (e.g., co-receptor:type I receptor, co-receptor:type II receptor, and co-receptor:co-receptor heteromultimers) may inhibit signaling of one or more TGF-beta superfamily ligands. For example, in some embodiments, a heteromultimer of the disclosure (e.g., co-receptor:type I receptor, co-receptor:type II receptor, and co-receptor:co-receptor heteromultimers) may inhibit signaling of one or more TGF-beta superfamily ligands in a cell-based assay (e.g., cell-based signaling assays as described herein). In some embodiments, heteromultimers of the disclosure are heterodimers.

In certain aspects, the disclosure relates to heteromultimers that comprise at least one endoglin polypeptide, which includes fragments, functional variants, and modified forms thereof, and at least one ALK1 polypeptide, which includes fragments, functional variants, and modified forms thereof. In some embodiments, the endoglin:ALK1 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 501, 502, 505, 506, 509, 510, 593, or 594. In some embodiments, the endoglin:ALK1 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-30 of SEQ ID NO: 501 and ends at any one of amino acids 330-346 of SEQ ID NO: 501. In some embodiments, the endoglin:ALK1 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-30 of SEQ ID NO: 505 and ends at any one of amino acids 330-346 of SEQ ID NO: 505. In some embodiments, the endoglin:ALK1 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-25 of SEQ ID NO: 509, and ends at any one of amino acids 148-164 of SEQ ID NO: 509. In some embodiments, the endoglin:ALK1 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 14 or 15. In some embodiments, the endoglin:ALK1 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 22-34 of SEQ ID NO: 14 and ends at any one of amino acids 95-118 of SEQ ID NO: 14. In certain preferred embodiments, endoglin:ALK1 heteromultimers are soluble. In some embodiments, an endoglin:ALK1 heteromultimer of the disclosure binds to one or more TGF-beta superfamily ligands (e.g., binds to one or more TGF-beta superfamily ligands with a K_{D} of at least 1×10⁻⁷). In some embodiments, an endoglin:ALK1 heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, an endoglin:ALK1 heteromultimer of the disclosure has a different TGF-beta ligand binding and/or inhibition profile (specificity) compared to a corresponding homomultimer (e.g., endoglin and ALK1 homomultimers). In some embodiments, an endoglin:ALK1 heteromultimer of the disclosure is a heterodimer.

In certain aspects, the disclosure relates to heteromultimers that comprise at least one endoglin polypeptide, which includes fragments, functional variants, and modified forms thereof, and at least one ALK2 polypeptide, which includes fragments, functional variants, and modified forms thereof. In some embodiments, the endoglin:ALK2 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 501, 502, 505, 506, 509, 510, 593, or 594. In some embodiments, the endoglin:ALK2 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-30 of SEQ ID NO: 501 and ends at any one of amino acids 330-346 of SEQ ID NO: 501. In some embodiments, the endoglin:ALK2 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-30 of SEQ ID NO: 505 and ends at any one of amino acids 330-346 of SEQ ID NO: 505. In some embodiments, the endoglin:ALK2 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-25 of SEQ ID NO: 509, and ends at any one of amino acids 148-164 of SEQ ID NO: 509. In some embodiments, the endoglin:ALK2 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: SEQ ID NO: 18 or 19. In some embodiments the endoglin:ALK2 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 21-35 of SEQ ID NO: 18 and ends at any one of amino acids 99-123 of SEQ ID NO: 18. In certain preferred embodiments, endoglin:ALK2 heteromultimers are soluble. In some embodiments, an endoglin:ALK2 heteromultimer of the disclosure binds to one or more TGF-beta superfamily ligands (e.g., binds to one or more TGF-beta superfamily ligands with a K_{D} of at least 1×10⁻⁷). In some embodiments, an endoglin:ALK2 heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, an endoglin:ALK2 heteromultimer of the disclosure has a different TGF-beta ligand binding and/or inhibition profile (specificity) compared to a corresponding homomultimer (e.g., endoglin and ALK2 homomultimers). In some embodiments, an endoglin:ALK2 heteromultimer of the disclosure is a heterodimer.

In certain aspects, the disclosure relates to heteromultimers that comprise at least one endoglin polypeptide, which includes fragments, functional variants, and modified forms thereof, and at least one ALK3 polypeptide, which includes fragments, functional variants, and modified forms thereof. In some embodiments, the endoglin:ALK3 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 501, 502, 505, 506, 509, 510, 593, or 594. In some embodiments, the endoglin:ALK3 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-30 of SEQ ID NO: 501 and ends at any one of amino acids 330-346 of SEQ ID NO: 501. In some embodiments, the endoglin:ALK3 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-30 of SEQ ID NO: 505 and ends at any one of amino acids 330-346 of SEQ ID NO: 505. In some embodiments, the endoglin:ALK3 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-25 of SEQ ID NO: 509, and ends at any one of amino acids 148-164 of SEQ ID NO: 509. In some embodiments, the endoglin:ALK3 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: SEQ ID NO: 22 or 23. In some embodiments the endoglin:ALK3 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 24-61 of SEQ ID NO: 22 and ends at any one of amino acids 130-152 of SEQ ID NO: 22. In certain preferred embodiments, endoglin:ALK3 heteromultimers are soluble. In some embodiments, an endoglin:ALK3 heteromultimer of the disclosure binds to one or more TGF-beta superfamily ligands (e.g., binds to one or more TGF-beta superfamily ligands with a K_{D} of at least 1×10⁻⁷). In some embodiments, an endoglin:ALK3 heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, an endoglin:ALK3 heteromultimer of the disclosure has a different TGF-beta ligand binding and/or inhibition profile (specificity) compared to a corresponding homomultimer (e.g., endoglin and ALK3 homomultimers). In some embodiments, an endoglin:ALK3 heteromultimer of the disclosure is a heterodimer.

In certain aspects, the disclosure relates to heteromultimers that comprise at least one endoglin polypeptide, which includes fragments, functional variants, and modified forms thereof, and at least one ALK4 polypeptide, which includes fragments, functional variants, and modified forms thereof. In some embodiments, the endoglin:ALK4 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 501, 502, 505, 506, 509, 510, 593, or 594. In some embodiments, the endoglin:ALK4 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-30 of SEQ ID NO: 501 and ends at any one of amino acids 330-346 of SEQ ID NO: 501. In some embodiments, the endoglin:ALK4 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-30 of SEQ ID NO: 505 and ends at any one of amino acids 330-346 of SEQ ID NO: 505. In some embodiments, the endoglin:ALK4 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-25 of SEQ ID NO: 509, and ends at any one of amino acids 148-164 of SEQ ID NO: 509. In some embodiments, the endoglin:ALK4 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: SEQ ID NO: 26, 27, 83, or 84. In some embodiments the endoglin:ALK4 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 24-34 of SEQ ID NO: 26 and ends at any one of amino acids 101-126 of SEQ ID NO: 26. In some embodiments the endoglin:ALK4 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 24-34 of SEQ ID NO: 83 and ends at any one of amino acids 101-126 of SEQ ID NO: 83. In certain preferred embodiments, endoglin:ALK4 heteromultimers are soluble. In some embodiments, an endoglin:ALK4 heteromultimer of the disclosure binds to one or more TGF-beta superfamily ligands (e.g., binds to one or more TGF-beta superfamily ligands with a K_{D} of at least 1×10⁻⁷). In some embodiments, an endoglin:ALK4 heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, an endoglin:ALK4 heteromultimer of the disclosure has a different TGF-beta ligand binding and/or inhibition profile (specificity) compared to a corresponding homomultimer (e.g., endoglin and ALK4 homomultimers). In some embodiments, an endoglin:ALK4 heteromultimer of the disclosure is a heterodimer.

In certain aspects, the disclosure relates to heteromultimers that comprise at least one endoglin polypeptide, which includes fragments, functional variants, and modified forms thereof, and at least one ALK5 polypeptide, which includes fragments, functional variants, and modified forms thereof. In some embodiments, the endoglin:ALK5 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 501, 502, 505, 506, 509, 510, 593, or 594. In some embodiments, the endoglin:ALK5 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-30 of SEQ ID NO: 501 and ends at any one of amino acids 330-346 of SEQ ID NO: 501. In some embodiments, the endoglin:ALK5 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-30 of SEQ ID NO: 505 and ends at any one of amino acids 330-346 of SEQ ID NO: 505. In some embodiments, the endoglin:ALK5 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-25 of SEQ ID NO: 509, and ends at any one of amino acids 148-164 of SEQ ID NO: 509. In some embodiments, the endoglin:ALK5 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: SEQ ID NO: 30, 31, 87, or 88. In some embodiments the endoglin:ALK5 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 25-36 of SEQ ID NO: 30 and ends at any one of amino acids 101-126 of SEQ ID NO: 30. In some embodiments the endoglin:ALK5 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 25-36 of SEQ ID NO: 87 and ends at any one of amino acids 101-130 of SEQ ID NO: 87. In certain preferred embodiments, endoglin:ALK5 heteromultimers are soluble. In some embodiments, an endoglin:ALK5 heteromultimer of the disclosure binds to one or more TGF-beta superfamily ligands (e.g., binds to one or more TGF-beta superfamily ligands with a K_{D} of at least 1×10⁻⁷). In some embodiments, an endoglin:ALK5 heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, an endoglin:ALK5 heteromultimer of the disclosure has a different TGF-beta ligand binding and/or inhibition profile (specificity) compared to a corresponding homomultimer (e.g., endoglin and ALK5 homomultimers). In some embodiments, an endoglin:ALK5 heteromultimer of the disclosure is a heterodimer.

In certain aspects, the disclosure relates to heteromultimers that comprise at least one endoglin polypeptide, which includes fragments, functional variants, and modified forms thereof, and at least one ALK6 polypeptide, which includes fragments, functional variants, and modified forms thereof. In some embodiments, the endoglin:ALK6 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 501, 502, 505, 506, 509, 510, 593, or 594. In some embodiments, the endoglin:ALK6 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-30 of SEQ ID NO: 501 and ends at any one of amino acids 330-346 of SEQ ID NO: 501. In some embodiments, the endoglin:ALK6 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-30 of SEQ ID NO: 505 and ends at any one of amino acids 330-346 of SEQ ID NO: 505. In some embodiments, the endoglin:ALK6 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-25 of SEQ ID NO: 509, and ends at any one of amino acids 148-164 of SEQ ID NO: 509. In some embodiments, the endoglin:ALK6 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: SEQ ID NO: 34, 35, 91, or 92. In some embodiments the endoglin:ALK6 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 14-32 of SEQ ID NO: 34 and ends at any one of amino acids 102-126 of SEQ ID NO: 34. In some embodiments the endoglin:ALK6 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-62 of SEQ ID NO: 91 and ends at any one of amino acids 132-156 of SEQ ID NO: 91. In certain preferred embodiments, endoglin:ALK6 heteromultimers are soluble. In some embodiments, an endoglin:ALK6 heteromultimer of the disclosure binds to one or more TGF-beta superfamily ligands (e.g., binds to one or more TGF-beta superfamily ligands with a K_{D} of at least 1×10⁻⁷). In some embodiments, an endoglin:ALK6 heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, an endoglin:ALK6 heteromultimer of the disclosure has a different TGF-beta ligand binding and/or inhibition profile (specificity) compared to a corresponding homomultimer (e.g., endoglin and ALK6 homomultimers). In some embodiments, an endoglin:ALK6 heteromultimer of the disclosure is a heterodimer.

In certain aspects, the disclosure relates to heteromultimers that comprise at least one endoglin polypeptide, which includes fragments, functional variants, and modified forms thereof, and at least one ALK7 polypeptide, which includes fragments, functional variants, and modified forms thereof. In some embodiments, the endoglin:ALK7 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 501, 502, 505, 506, 509, 510, 593, or 594. In some embodiments, the endoglin:ALK7 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-30 of SEQ ID NO: 501 and ends at any one of amino acids 330-346 of SEQ ID NO: 501. In some embodiments, the endoglin:ALK7 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-30 of SEQ ID NO: 505 and ends at any one of amino acids 330-346 of SEQ ID NO: 505. In some embodiments, the endoglin:ALK7 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-25 of SEQ ID NO: 509, and ends at any one of amino acids 148-164 of SEQ ID NO: 509. In some embodiments, the endoglin:ALK7 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: SEQ ID NO: 38, 39, 301, 302, 305, 306, 309, 310, or 313. In some embodiments the endoglin:ALK7 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 21-28 of SEQ ID NO: 38 and ends at any one of amino acids 92-113 of SEQ ID NO: 38. In some embodiments the endoglin:ALK7 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-13 of SEQ ID NO: 301 and ends at any one of amino acids 42-63 of SEQ ID NO: 301. In some embodiments the endoglin:ALK7 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 21-28 of SEQ ID NO: 305 and ends at any one of amino acids 411-413 of SEQ ID NO: 305. In some embodiments the endoglin:ALK7 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 21-28 of SEQ ID NO: 309 and ends at any one of amino acids 334-336 of SEQ ID NO: 309. In certain preferred embodiments, endoglin:ALK7 heteromultimers are soluble. In some embodiments, an endoglin:ALK7 heteromultimer of the disclosure binds to one or more TGF-beta superfamily ligands (e.g., binds to one or more TGF-beta superfamily ligands with a K_{D} of at least 1×10⁻⁷). In some embodiments, an endoglin:ALK7 heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, an endoglin:ALK7 heteromultimer of the disclosure has a different TGF-beta ligand binding and/or inhibition profile (specificity) compared to a corresponding homomultimer (e.g., endoglin and ALK7 homomultimers). In some embodiments, an endoglin:ALK7 heteromultimer of the disclosure is a heterodimer.

In certain aspects, the disclosure relates to heteromultimers that comprise at least one betaglycan polypeptide, which includes fragments, functional variants, and modified forms thereof, and at least one ALK1 polypeptide, which includes fragments, functional variants, and modified forms thereof. In some embodiments, the betaglycan:ALK1 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 585, 586, 589, or 590. In some embodiments, the betaglycan:ALK1 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 21-28 of SEQ ID NO: 585 and ends at any one of amino acids 381-787 of SEQ ID NO: 585. In some embodiments, the betaglycan:ALK1 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 21-28 of SEQ ID NO: 589 and ends at any one of amino acids 380-786 of SEQ ID NO: 589. In some embodiments, the betaglycan:ALK1 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 14 or 15. In some embodiments, the betaglycan:ALK1 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 22-34 of SEQ ID NO: 14 and ends at any one of amino acids 95-118 of SEQ ID NO: 14. In certain preferred embodiments, betaglycan:ALK1 heteromultimers are soluble. In some embodiments, an betaglycan:ALK1 heteromultimer of the disclosure binds to one or more TGF-beta superfamily ligands (e.g., binds to one or more TGF-beta superfamily ligands with a K_{D} of at least 1×10⁻⁷). In some embodiments, an betaglycan:ALK1 heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, an betaglycan:ALK1 heteromultimer of the disclosure has a different TGF-beta ligand binding and/or inhibition profile (specificity) compared to a corresponding homomultimer (e.g., betaglycan and ALK1 homomultimers). In some embodiments, an betaglycan:ALK1 heteromultimer of the disclosure is a heterodimer.

In certain aspects, the disclosure relates to heteromultimers that comprise at least one betaglycan polypeptide, which includes fragments, functional variants, and modified forms thereof, and at least one ALK2 polypeptide, which includes fragments, functional variants, and modified forms thereof. In some embodiments, the betaglycan:ALK2 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 585, 586, 589, or 590. In some embodiments, the betaglycan:ALK2 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 21-28 of SEQ ID NO: 585 and ends at any one of amino acids 381-787 of SEQ ID NO: 585. In some embodiments, the betaglycan:ALK2 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 21-28 of SEQ ID NO: 589 and ends at any one of amino acids 380-786 of SEQ ID NO: 589. In some embodiments, the betaglycan:ALK2 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: SEQ ID NO: 18 or 19. In some embodiments the betaglycan:ALK2 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 21-35 of SEQ ID NO: 18 and ends at any one of amino acids 99-123 of SEQ ID NO: 18. In certain preferred embodiments, betaglycan:ALK2 heteromultimers are soluble. In some embodiments, an betaglycan:ALK2 heteromultimer of the disclosure binds to one or more TGF-beta superfamily ligands (e.g., binds to one or more TGF-beta superfamily ligands with a K_{D} of at least 1×10⁻⁷). In some embodiments, an betaglycan:ALK2 heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, an betaglycan:ALK2 heteromultimer of the disclosure has a different TGF-beta ligand binding and/or inhibition profile (specificity) compared to a corresponding homomultimer (e.g., betaglycan and ALK2 homomultimers). In some embodiments, an betaglycan:ALK2 heteromultimer of the disclosure is a heterodimer.

In certain aspects, the disclosure relates to heteromultimers that comprise at least one betaglycan polypeptide, which includes fragments, functional variants, and modified forms thereof, and at least one ALK3 polypeptide, which includes fragments, functional variants, and modified forms thereof. In some embodiments, the betaglycan:ALK3 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 585, 586, 589, or 590. In some embodiments, the betaglycan:ALK3 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 21-28 of SEQ ID NO: 585 and ends at any one of amino acids 381-787 of SEQ ID NO: 585. In some embodiments, the betaglycan:ALK3 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 21-28 of SEQ ID NO: 589 and ends at any one of amino acids 380-786 of SEQ ID NO: 589. In some embodiments, the betaglycan:ALK3 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: SEQ ID NO: 22 or 23. In some embodiments the betaglycan:ALK3 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 24-61 of SEQ ID NO: 22 and ends at any one of amino acids 130-152 of SEQ ID NO: 22. In certain preferred embodiments, betaglycan:ALK3 heteromultimers are soluble. In some embodiments, an betaglycan:ALK3 heteromultimer of the disclosure binds to one or more TGF-beta superfamily ligands (e.g., binds to one or more TGF-beta superfamily ligands with a K_{D} of at least 1×10⁻⁷). In some embodiments, an betaglycan:ALK3 heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, an betaglycan:ALK3 heteromultimer of the disclosure has a different TGF-beta ligand binding and/or inhibition profile (specificity) compared to a corresponding homomultimer (e.g., betaglycan and ALK3 homomultimers). In some embodiments, an betaglycan:ALK3 heteromultimer of the disclosure is a heterodimer.

In certain aspects, the disclosure relates to heteromultimers that comprise at least one betaglycan polypeptide, which includes fragments, functional variants, and modified forms thereof, and at least one ALK4 polypeptide, which includes fragments, functional variants, and modified forms thereof. In some embodiments, the betaglycan:ALK4 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 585, 586, 589, or 590. In some embodiments, the betaglycan:ALK4 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 21-28 of SEQ ID NO: 585 and ends at any one of amino acids 381-787 of SEQ ID NO: 585. In some embodiments, the betaglycan:ALK4 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 21-28 of SEQ ID NO: 589 and ends at any one of amino acids 380-786 of SEQ ID NO: 589. In some embodiments, the betaglycan:ALK4 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: SEQ ID NO: 26, 27, 83, or 84. In some embodiments the betaglycan:ALK4 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 24-34 of SEQ ID NO: 26 and ends at any one of amino acids 101-126 of SEQ ID NO: 26. In some embodiments the betaglycan:ALK4 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 24-34 of SEQ ID NO: 83 and ends at any one of amino acids 101-126 of SEQ ID NO: 83. In certain preferred embodiments, betaglycan:ALK4 heteromultimers are soluble. In some embodiments, an betaglycan:ALK4 heteromultimer of the disclosure binds to one or more TGF-beta superfamily ligands (e.g., binds to one or more TGF-beta superfamily ligands with a K_{D} of at least 1×10⁻⁷). In some embodiments, an betaglycan:ALK4 heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, an betaglycan:ALK4 heteromultimer of the disclosure has a different TGF-beta ligand binding and/or inhibition profile (specificity) compared to a corresponding homomultimer (e.g., betaglycan and ALK4 homomultimers). In some embodiments, an betaglycan:ALK4 heteromultimer of the disclosure is a heterodimer.

In certain aspects, the disclosure relates to heteromultimers that comprise at least one betaglycan polypeptide, which includes fragments, functional variants, and modified forms thereof, and at least one ALK5 polypeptide, which includes fragments, functional variants, and modified forms thereof. In some embodiments, the betaglycan:ALK5 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 585, 586, 589, or 590. In some embodiments, the betaglycan:ALK5 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 21-28 of SEQ ID NO: 585 and ends at any one of amino acids 381-787 of SEQ ID NO: 585. In some embodiments, the betaglycan:ALK5 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 21-28 of SEQ ID NO: 589 and ends at any one of amino acids 380-786 of SEQ ID NO: 589. In some embodiments, the betaglycan:ALK5 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: SEQ ID NO: 30, 31, 87, or 88. In some embodiments the betaglycan:ALK5 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 25-36 of SEQ ID NO: 30 and ends at any one of amino acids 101-126 of SEQ ID NO: 30. In some embodiments the betaglycan:ALK5 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 25-36 of SEQ ID NO: 87 and ends at any one of amino acids 101-130 of SEQ ID NO: 87. In certain preferred embodiments, betaglycan:ALK5 heteromultimers are soluble. In some embodiments, an betaglycan:ALK5 heteromultimer of the disclosure binds to one or more TGF-beta superfamily ligands (e.g., binds to one or more TGF-beta superfamily ligands with a K_{D} of at least 1×10⁻⁷). In some embodiments, an betaglycan:ALK5 heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, an betaglycan:ALK5 heteromultimer of the disclosure has a different TGF-beta ligand binding and/or inhibition profile (specificity) compared to a corresponding homomultimer (e.g., betaglycan and ALK5 homomultimers). In some embodiments, an betaglycan:ALK5 heteromultimer of the disclosure is a heterodimer.

In certain aspects, the disclosure relates to heteromultimers that comprise at least one betaglycan polypeptide, which includes fragments, functional variants, and modified forms thereof, and at least one ALK6 polypeptide, which includes fragments, functional variants, and modified forms thereof. In some embodiments, the betaglycan:ALK6 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 585, 586, 589, or 590. In some embodiments, the betaglycan:ALK6 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 21-28 of SEQ ID NO: 585 and ends at any one of amino acids 381-787 of SEQ ID NO: 585. In some embodiments, the betaglycan:ALK6 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 21-28 of SEQ ID NO: 589 and ends at any one of amino acids 380-786 of SEQ ID NO: 589. In some embodiments, the betaglycan:ALK6 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: SEQ ID NO: 34, 35, 91, or 92. In some embodiments the betaglycan:ALK6 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 14-32 of SEQ ID NO: 34 and ends at any one of amino acids 102-126 of SEQ ID NO: 34. In some embodiments the betaglycan:ALK6 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-62 of SEQ ID NO: 91 and ends at any one of amino acids 132-156 of SEQ ID NO: 91. In certain preferred embodiments, betaglycan:ALK6 heteromultimers are soluble. In some embodiments, an betaglycan:ALK6 heteromultimer of the disclosure binds to one or more TGF-beta superfamily ligands (e.g., binds to one or more TGF-beta superfamily ligands with a K_{D} of at least 1×10⁻⁷). In some embodiments, an betaglycan:ALK6 heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, an betaglycan:ALK6 heteromultimer of the disclosure has a different TGF-beta ligand binding and/or inhibition profile (specificity) compared to a corresponding homomultimer (e.g., betaglycan and ALK6 homomultimers). In some embodiments, an betaglycan:ALK6 heteromultimer of the disclosure is a heterodimer.

In certain aspects, the disclosure relates to heteromultimers that comprise at least one betaglycan polypeptide, which includes fragments, functional variants, and modified forms thereof, and at least one ALK7 polypeptide, which includes fragments, functional variants, and modified forms thereof. In some embodiments, the betaglycan:ALK7 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 585, 586, 589, or 590. In some embodiments, the betaglycan:ALK7 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 21-28 of SEQ ID NO: 585 and ends at any one of amino acids 381-787 of SEQ ID NO: 585. In some embodiments, the betaglycan:ALK7 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 21-28 of SEQ ID NO: 589 and ends at any one of amino acids 380-786 of SEQ ID NO: 589. In some embodiments, the betaglycan:ALK7 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: SEQ ID NO: 38, 39, 301, 302, 305, 306, 309, 310, or 313. In some embodiments the betaglycan:ALK7 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 21-28 of SEQ ID NO: 38 and ends at any one of amino acids 92-113 of SEQ ID NO: 38. In some embodiments the betaglycan:ALK7 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-13 of SEQ ID NO: 301 and ends at any one of amino acids 42-63 of SEQ ID NO: 301. In some embodiments the betaglycan:ALK7 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 21-28 of SEQ ID NO: 305 and ends at any one of amino acids 411-413 of SEQ ID NO: 305. In some embodiments the betaglycan:ALK7 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 21-28 of SEQ ID NO: 309 and ends at any one of amino acids 334-336 of SEQ ID NO: 309. In certain preferred embodiments, betaglycan:ALK7 heteromultimers are soluble. In some embodiments, an betaglycan:ALK7 heteromultimer of the disclosure binds to one or more TGF-beta superfamily ligands (e.g., binds to one or more TGF-beta superfamily ligands with a K_{D} of at least 1×10⁻⁷). In some embodiments, an betaglycan:ALK7 heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, an betaglycan:ALK7 heteromultimer of the disclosure has a different TGF-beta ligand binding and/or inhibition profile (specificity) compared to a corresponding homomultimer (e.g., betaglycan and ALK7 homomultimers). In some embodiments, an betaglycan:ALK7 heteromultimer of the disclosure is a heterodimer.

In certain aspects, the disclosure relates to heteromultimers that comprise at least one Cripto-1 polypeptide, which includes fragments, functional variants, and modified forms thereof, and at least one ALK1 polypeptide, which includes fragments, functional variants, and modified forms thereof. In some embodiments, the Cripto-1:ALK1 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 513, 514, 517, or 518. In some embodiments, the Cripto-1:ALK1 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 31-82 of SEQ ID NO: 513 and ends at any one of amino acids 172-188 of SEQ ID NO: 513. In some embodiments, the Cripto-1:ALK1 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 15-66 of SEQ ID NO: 517, and ends at any one of amino acids 156-172 of SEQ ID NO: 517. In some embodiments, the Cripto-1:ALK1 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 14 or 15. In some embodiments, the Cripto-1:ALK1 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 22-34 of SEQ ID NO: 14 and ends at any one of amino acids 95-118 of SEQ ID NO: 14. In certain preferred embodiments, Cripto-1:ALK1 heteromultimers are soluble. In some embodiments, an Cripto-1:ALK1 heteromultimer of the disclosure binds to one or more TGF-beta superfamily ligands (e.g., binds to one or more TGF-beta superfamily ligands with a K_{D} of at least 1×10⁻⁷). In some embodiments, an Cripto-1:ALK1 heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, an Cripto-1:ALK1 heteromultimer of the disclosure has a different TGF-beta ligand binding and/or inhibition profile (specificity) compared to a corresponding homomultimer (e.g., Cripto-1 and ALK1 homomultimers). In some embodiments, an Cripto-1:ALK1 heteromultimer of the disclosure is a heterodimer.

In certain aspects, the disclosure relates to heteromultimers that comprise at least one Cripto-1 polypeptide, which includes fragments, functional variants, and modified forms thereof, and at least one ALK2 polypeptide, which includes fragments, functional variants, and modified forms thereof. In some embodiments, the Cripto-1:ALK2 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 513, 514, 517, or 518. In some embodiments, the Cripto-1:ALK2 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 31-82 of SEQ ID NO: 513 and ends at any one of amino acids 172-188 of SEQ ID NO: 513. In some embodiments, the Cripto-1:ALK2 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 15-66 of SEQ ID NO: 517, and ends at any one of amino acids 156-172 of SEQ ID NO: 517. In some embodiments, the Cripto-1:ALK2 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: SEQ ID NO: 18 or 19. In some embodiments the Cripto-1:ALK2 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 21-35 of SEQ ID NO: 18 and ends at any one of amino acids 99-123 of SEQ ID NO: 18. In certain preferred embodiments, Cripto-1:ALK2 heteromultimers are soluble. In some embodiments, an Cripto-1:ALK2 heteromultimer of the disclosure binds to one or more TGF-beta superfamily ligands (e.g., binds to one or more TGF-beta superfamily ligands with a K_{D} of at least 1×10⁻⁷). In some embodiments, an Cripto-1:ALK2 heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, an Cripto-1:ALK2 heteromultimer of the disclosure has a different TGF-beta ligand binding and/or inhibition profile (specificity) compared to a corresponding homomultimer (e.g., Cripto-1 and ALK2 homomultimers). In some embodiments, an Cripto-1:ALK2 heteromultimer of the disclosure is a heterodimer.

In certain aspects, the disclosure relates to heteromultimers that comprise at least one Cripto-1 polypeptide, which includes fragments, functional variants, and modified forms thereof, and at least one ALK3 polypeptide, which includes fragments, functional variants, and modified forms thereof. In some embodiments, the Cripto-1:ALK3 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 513, 514, 517, or 518. In some embodiments, the Cripto-1:ALK3 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 31-82 of SEQ ID NO: 513 and ends at any one of amino acids 172-188 of SEQ ID NO: 513. In some embodiments, the Cripto-1:ALK3 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 15-66 of SEQ ID NO: 517, and ends at any one of amino acids 156-172 of SEQ ID NO: 517. In some embodiments, the Cripto-1:ALK3 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: SEQ ID NO: 22 or 23. In some embodiments the Cripto-1:ALK3 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 24-61 of SEQ ID NO: 22 and ends at any one of amino acids 130-152 of SEQ ID NO: 22. In certain preferred embodiments, Cripto-1:ALK3 heteromultimers are soluble. In some embodiments, an Cripto-1:ALK3 heteromultimer of the disclosure binds to one or more TGF-beta superfamily ligands (e.g., binds to one or more TGF-beta superfamily ligands with a K_{D} of at least 1×10⁻⁷). In some embodiments, an Cripto-1:ALK3 heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, an Cripto-1:ALK3 heteromultimer of the disclosure has a different TGF-beta ligand binding and/or inhibition profile (specificity) compared to a corresponding homomultimer (e.g., Cripto-1 and ALK3 homomultimers). In some embodiments, an Cripto-1:ALK3 heteromultimer of the disclosure is a heterodimer.

In certain aspects, the disclosure relates to heteromultimers that comprise at least one Cripto-1 polypeptide, which includes fragments, functional variants, and modified forms thereof, and at least one ALK4 polypeptide, which includes fragments, functional variants, and modified forms thereof. In some embodiments, the Cripto-1:ALK4 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 513, 514, 517, or 518. In some embodiments, the Cripto-1:ALK4 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 31-82 of SEQ ID NO: 513 and ends at any one of amino acids 172-188 of SEQ ID NO: 513. In some embodiments, the Cripto-1:ALK4 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 15-66 of SEQ ID NO: 517, and ends at any one of amino acids 156-172 of SEQ ID NO: 517. In some embodiments, the Cripto-1:ALK4 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: SEQ ID NO: 26, 27, 83, or 84. In some embodiments the Cripto-1:ALK4 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 24-34 of SEQ ID NO: 26 and ends at any one of amino acids 101-126 of SEQ ID NO: 26. In some embodiments the Cripto-1:ALK4 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 24-34 of SEQ ID NO: 83 and ends at any one of amino acids 101-126 of SEQ ID NO: 83. In certain preferred embodiments, Cripto-1:ALK4 heteromultimers are soluble. In some embodiments, a Cripto-1:ALK4 heteromultimer of the disclosure binds to one or more TGF-beta superfamily ligands (e.g., binds to one or more TGF-beta superfamily ligands with a K_{D} of at least 1×10⁻⁷). In some embodiments, a Cripto-1:ALK4 heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a Cripto-1:ALK4 heteromultimer of the disclosure has a different TGF-beta ligand binding and/or inhibition profile (specificity) compared to a corresponding homomultimer (e.g., Cripto-1 and ALK4 homomultimers). In some embodiments, a Cripto-1:ALK4 heteromultimer of the disclosure is a heterodimer.

In certain aspects, the disclosure relates to heteromultimers that comprise at least one Cripto-1 polypeptide, which includes fragments, functional variants, and modified forms thereof, and at least one ALK5 polypeptide, which includes fragments, functional variants, and modified forms thereof. In some embodiments, the Cripto-1:ALK5 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 513, 514, 517, or 518. In some embodiments, the Cripto-1:ALK5 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 31-82 of SEQ ID NO: 513 and ends at any one of amino acids 172-188 of SEQ ID NO: 513. In some embodiments, the Cripto-1:ALK5 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 15-66 of SEQ ID NO: 517, and ends at any one of amino acids 156-172 of SEQ ID NO: 517. In some embodiments, the Cripto-1:ALK5 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: SEQ ID NO: 30, 31, 87, or 88. In some embodiments the Cripto-1:ALK5 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 25-36 of SEQ ID NO: 30 and ends at any one of amino acids 101-126 of SEQ ID NO: 30. In some embodiments the Cripto-1:ALK5 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 25-36 of SEQ ID NO: 87 and ends at any one of amino acids 101-130 of SEQ ID NO: 87. In certain preferred embodiments, Cripto-1:ALK5 heteromultimers are soluble. In some embodiments, a Cripto-1:ALK5 heteromultimer of the disclosure binds to one or more TGF-beta superfamily ligands (e.g., binds to one or more TGF-beta superfamily ligands with a K_{D} of at least 1×10⁻⁷). In some embodiments, a Cripto-1:ALK5 heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a Cripto-1:ALK5 heteromultimer of the disclosure has a different TGF-beta ligand binding and/or inhibition profile (specificity) compared to a corresponding homomultimer (e.g., Cripto-1 and ALK5 homomultimers). In some embodiments, a Cripto-1:ALK5 heteromultimer of the disclosure is a heterodimer.

In certain aspects, the disclosure relates to heteromultimers that comprise at least one Cripto-1 polypeptide, which includes fragments, functional variants, and modified forms thereof, and at least one ALK6 polypeptide, which includes fragments, functional variants, and modified forms thereof. In some embodiments, the Cripto-1:ALK6 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 513, 514, 517, or 518. In some embodiments, the Cripto-1:ALK6 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 31-82 of SEQ ID NO: 513 and ends at any one of amino acids 172-188 of SEQ ID NO: 513. In some embodiments, the Cripto-1:ALK6 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 15-66 of SEQ ID NO: 517, and ends at any one of amino acids 156-172 of SEQ ID NO: 517. In some embodiments, the Cripto-1:ALK6 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: SEQ ID NO: 34, 35, 91, or 92. In some embodiments the Cripto-1:ALK6 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 14-32 of SEQ ID NO: 34 and ends at any one of amino acids 102-126 of SEQ ID NO: 34. In some embodiments the Cripto-1:ALK6 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-62 of SEQ ID NO: 91 and ends at any one of amino acids 132-156 of SEQ ID NO: 91. In certain preferred embodiments, Cripto-1:ALK6 heteromultimers are soluble. In some embodiments, a Cripto-1:ALK6 heteromultimer of the disclosure binds to one or more TGF-beta superfamily ligands (e.g., binds to one or more TGF-beta superfamily ligands with a K_{D} of at least 1×10⁻⁷). In some embodiments, a Cripto-1:ALK6 heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a Cripto-1:ALK6 heteromultimer of the disclosure has a different TGF-beta ligand binding and/or inhibition profile (specificity) compared to a corresponding homomultimer (e.g., Cripto-1 and 6 homomultimers). In some embodiments, a Cripto-1:ALK6 heteromultimer of the disclosure is a heterodimer.

In certain aspects, the disclosure relates to heteromultimers that comprise at least one Cripto-1 polypeptide, which includes fragments, functional variants, and modified forms thereof, and at least one ALK7 polypeptide, which includes fragments, functional variants, and modified forms thereof. In some embodiments, the Cripto-1:ALK7 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 513, 514, 517, or 518. In some embodiments, the Cripto-1:ALK7 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 31-82 of SEQ ID NO: 513 and ends at any one of amino acids 172-188 of SEQ ID NO: 513. In some embodiments, the Cripto-1:ALK7 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 15-66 of SEQ ID NO: 517, and ends at any one of amino acids 156-172 of SEQ ID NO: 517. In some embodiments, the Cripto-1:ALK7 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: SEQ ID NO: 38, 39, 301, 302, 305, 306, 309, 310, or 313. In some embodiments the Cripto-1:ALK7 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 21-28 of SEQ ID NO: 38 and ends at any one of amino acids 92-113 of SEQ ID NO: 38. In some embodiments the Cripto-1:ALK7 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-13 of SEQ ID NO: 301 and ends at any one of amino acids 42-63 of SEQ ID NO: 301. In some embodiments the Cripto-1:ALK7 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 21-28 of SEQ ID NO: 305 and ends at any one of amino acids 411-413 of SEQ ID NO: 305. In some embodiments the Cripto-1:ALK7 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 21-28 of SEQ ID NO: 309 and ends at any one of amino acids 334-336 of SEQ ID NO: 309. In certain preferred embodiments, Cripto-1:ALK7 heteromultimers are soluble. In some embodiments, a Cripto-1:ALK7 heteromultimer of the disclosure binds to one or more TGF-beta superfamily ligands (e.g., binds to one or more TGF-beta superfamily ligands with a K_{D} of at least 1×10⁻⁷). In some embodiments, a Cripto-1:ALK7 heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a Cripto-1:ALK7 heteromultimer of the disclosure has a different TGF-beta ligand binding and/or inhibition profile (specificity) compared to a corresponding homomultimer (e.g., Cripto-1 and ALK7 homomultimers). In some embodiments, a Cripto-1:ALK7 heteromultimer of the disclosure is a heterodimer.

In certain aspects, the disclosure relates to heteromultimers that comprise at least one Cryptic protein polypeptide, which includes fragments, functional variants, and modified forms thereof, and at least one ALK1 polypeptide, which includes fragments, functional variants, and modified forms thereof. In some embodiments, the Cryptic protein:ALK1 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 521, 522, 525, 526, 529, or 530. In some embodiments, the Cryptic protein:ALK1 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-90 of SEQ ID NO: 521 and ends at any one of amino acids 157-233 of SEQ ID NO: 521. In some embodiments, the Cryptic protein:ALK1 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-30 of SEQ ID NO: 525 and ends at any one of amino acids 82-191 of SEQ ID NO: 525. In some embodiments, the Cryptic protein:ALK1 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-30 of SEQ ID NO: 529, and ends at any one of amino acids 82-148 of SEQ ID NO: 529. In some embodiments, the Cryptic protein:ALK1 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 14 or 15. In some embodiments, the Cryptic protein:ALK1 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 22-34 of SEQ ID NO: 14 and ends at any one of amino acids 95-118 of SEQ ID NO: 14. In certain preferred embodiments, Cryptic protein:ALK1 heteromultimers are soluble. In some embodiments, a Cryptic protein:ALK1 heteromultimer of the disclosure binds to one or more TGF-beta superfamily ligands (e.g., binds to one or more TGF-beta superfamily ligands with a K_{D} of at least 1×10⁻⁷). In some embodiments, a Cryptic protein:ALK1 heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a Cryptic protein:ALK1 heteromultimer of the disclosure has a different TGF-beta ligand binding and/or inhibition profile (specificity) compared to a corresponding homomultimer (e.g., Cryptic protein and ALK1 homomultimers). In some embodiments, a Cryptic protein:ALK1 heteromultimer of the disclosure is a heterodimer.

In certain aspects, the disclosure relates to heteromultimers that comprise at least one Cryptic protein polypeptide, which includes fragments, functional variants, and modified forms thereof, and at least one ALK2 polypeptide, which includes fragments, functional variants, and modified forms thereof. In some embodiments, the Cryptic protein:ALK2 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 521, 522, 525, 526, 529, or 530. In some embodiments, the Cryptic protein:ALK2 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-90 of SEQ ID NO: 521 and ends at any one of amino acids 157-233 of SEQ ID NO: 521. In some embodiments, the Cryptic protein:ALK2 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-30 of SEQ ID NO: 525 and ends at any one of amino acids 82-191 of SEQ ID NO: 525. In some embodiments, the Cryptic protein:ALK2 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-30 of SEQ ID NO: 529, and ends at any one of amino acids 82-148 of SEQ ID NO: 529. In some embodiments, the Cryptic protein:ALK2 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: SEQ ID NO: 18 or 19. In some embodiments the Cryptic protein:ALK2 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 21-35 of SEQ ID NO: 18 and ends at any one of amino acids 99-123 of SEQ ID NO: 18. In certain preferred embodiments, Cryptic protein:ALK2 heteromultimers are soluble. In some embodiments, a Cryptic protein:ALK2 heteromultimer of the disclosure binds to one or more TGF-beta superfamily ligands (e.g., binds to one or more TGF-beta superfamily ligands with a K_{D} of at least 1×10⁻⁷). In some embodiments, a Cryptic protein:ALK2 heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a Cryptic protein:ALK2 heteromultimer of the disclosure has a different TGF-beta ligand binding and/or inhibition profile (specificity) compared to a corresponding homomultimer (e.g., Cryptic protein and ALK2 homomultimers). In some embodiments, a Cryptic protein:ALK2 heteromultimer of the disclosure is a heterodimer.

In certain aspects, the disclosure relates to heteromultimers that comprise at least one Cryptic protein polypeptide, which includes fragments, functional variants, and modified forms thereof, and at least one ALK3 polypeptide, which includes fragments, functional variants, and modified forms thereof. In some embodiments, the Cryptic protein:ALK3 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 521, 522, 525, 526, 529, or 530. In some embodiments, the Cryptic protein:ALK3 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-90 of SEQ ID NO: 521 and ends at any one of amino acids 157-233 of SEQ ID NO: 521. In some embodiments, the Cryptic protein:ALK3 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-30 of SEQ ID NO: 525 and ends at any one of amino acids 82-191 of SEQ ID NO: 525. In some embodiments, the Cryptic protein:ALK3 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-30 of SEQ ID NO: 529, and ends at any one of amino acids 82-148 of SEQ ID NO: 529. In some embodiments, the Cryptic protein:ALK3 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: SEQ ID NO: 22 or 23. In some embodiments the Cryptic protein:ALK3 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 24-61 of SEQ ID NO: 22 and ends at any one of amino acids 130-152 of SEQ ID NO: 22. In certain preferred embodiments, Cryptic protein:ALK3 heteromultimers are soluble. In some embodiments, a Cryptic protein:ALK3 heteromultimer of the disclosure binds to one or more TGF-beta superfamily ligands (e.g., binds to one or more TGF-beta superfamily ligands with a K_{D} of at least 1×10⁻⁷). In some embodiments, a Cryptic protein:ALK3 heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a Cryptic protein:ALK3 heteromultimer of the disclosure has a different TGF-beta ligand binding and/or inhibition profile (specificity) compared to a corresponding homomultimer (e.g., Cryptic protein and ALK3 homomultimers). In some embodiments, a Cryptic protein:ALK3 heteromultimer of the disclosure is a heterodimer.

In certain aspects, the disclosure relates to heteromultimers that comprise at least one Cryptic protein polypeptide, which includes fragments, functional variants, and modified forms thereof, and at least one ALK4 polypeptide, which includes fragments, functional variants, and modified forms thereof. In some embodiments, the Cryptic protein:ALK4 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 521, 522, 525, 526, 529, or 530. In some embodiments, the Cryptic protein:ALK4 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-90 of SEQ ID NO: 521 and ends at any one of amino acids 157-233 of SEQ ID NO: 521. In some embodiments, the Cryptic protein:ALK4 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-30 of SEQ ID NO: 525 and ends at any one of amino acids 82-191 of SEQ ID NO: 525. In some embodiments, the Cryptic protein:ALK4 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-30 of SEQ ID NO: 529, and ends at any one of amino acids 82-148 of SEQ ID NO: 529. In some embodiments, the Cryptic protein:ALK4 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: SEQ ID NO: 26, 27, 83, or 84. In some embodiments the Cryptic protein:ALK4 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 24-34 of SEQ ID NO: 26 and ends at any one of amino acids 101-126 of SEQ ID NO: 26. In some embodiments the Cryptic protein:ALK4 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 24-34 of SEQ ID NO: 83 and ends at any one of amino acids 101-126 of SEQ ID NO: 83. In certain preferred embodiments, Cryptic protein:ALK4 heteromultimers are soluble. In some embodiments, a Cryptic protein:ALK4 heteromultimer of the disclosure binds to one or more TGF-beta superfamily ligands (e.g., binds to one or more TGF-beta superfamily ligands with a K_{D} of at least 1×10⁻⁷). In some embodiments, a Cryptic protein:ALK4 heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a Cryptic protein:ALK4 heteromultimer of the disclosure has a different TGF-beta ligand binding and/or inhibition profile (specificity) compared to a corresponding homomultimer (e.g., Cryptic protein and ALK4 homomultimers). In some embodiments, a Cryptic protein:ALK4 heteromultimer of the disclosure is a heterodimer.

In certain aspects, the disclosure relates to heteromultimers that comprise at least one Cryptic protein polypeptide, which includes fragments, functional variants, and modified forms thereof, and at least one ALK5 polypeptide, which includes fragments, functional variants, and modified forms thereof. In some embodiments, the Cryptic protein:ALK5 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 521, 522, 525, 526, 529, or 530. In some embodiments, the Cryptic protein:ALK5 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-90 of SEQ ID NO: 521 and ends at any one of amino acids 157-233 of SEQ ID NO: 521. In some embodiments, the Cryptic protein:ALK5 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-30 of SEQ ID NO: 525 and ends at any one of amino acids 82-191 of SEQ ID NO: 525. In some embodiments, the Cryptic protein:ALK5 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-30 of SEQ ID NO: 529, and ends at any one of amino acids 82-148 of SEQ ID NO: 529. In some embodiments, the Cryptic protein:ALK5 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: SEQ ID NO: 30, 31, 87, or 88. In some embodiments the Cryptic protein:ALK5 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 25-36 of SEQ ID NO: 30 and ends at any one of amino acids 101-126 of SEQ ID NO: 30. In some embodiments the Cryptic protein:ALK5 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 25-36 of SEQ ID NO: 87 and ends at any one of amino acids 101-130 of SEQ ID NO: 87. In certain preferred embodiments, Cryptic protein:ALK5 heteromultimers are soluble. In some embodiments, a Cryptic protein:ALK5 heteromultimer of the disclosure binds to one or more TGF-beta superfamily ligands (e.g., binds to one or more TGF-beta superfamily ligands with a K_{D} of at least 1×10⁻⁷). In some embodiments, a Cryptic protein:ALK5 heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a Cryptic protein:ALK5 heteromultimer of the disclosure has a different TGF-beta ligand binding and/or inhibition profile (specificity) compared to a corresponding homomultimer (e.g., Cryptic protein and ALK5 homomultimers). In some embodiments, a Cryptic protein:ALK5 heteromultimer of the disclosure is a heterodimer.

In certain aspects, the disclosure relates to heteromultimers that comprise at least one Cryptic protein polypeptide, which includes fragments, functional variants, and modified forms thereof, and at least one ALK6 polypeptide, which includes fragments, functional variants, and modified forms thereof. In some embodiments, the Cryptic protein:ALK6 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 521, 522, 525, 526, 529, or 530. In some embodiments, the Cryptic protein:ALK6 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-90 of SEQ ID NO: 521 and ends at any one of amino acids 157-233 of SEQ ID NO: 521. In some embodiments, the Cryptic protein:ALK6 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-30 of SEQ ID NO: 525 and ends at any one of amino acids 82-191 of SEQ ID NO: 525. In some embodiments, the Cryptic protein:ALK6 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-30 of SEQ ID NO: 529, and ends at any one of amino acids 82-148 of SEQ ID NO: 529. In some embodiments, the Cryptic protein:ALK6 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: SEQ ID NO: 34, 35, 91, or 92. In some embodiments the Cryptic protein:ALK6 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 14-32 of SEQ ID NO: 34 and ends at any one of amino acids 102-126 of SEQ ID NO: 34. In some embodiments the Cryptic protein:ALK6 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-62 of SEQ ID NO: 91 and ends at any one of amino acids 132-156 of SEQ ID NO: 91. In certain preferred embodiments, Cryptic protein:ALK6 heteromultimers are soluble. In some embodiments, a Cryptic protein:ALK6 heteromultimer of the disclosure binds to one or more TGF-beta superfamily ligands (e.g., binds to one or more TGF-beta superfamily ligands with a K_{D} of at least 1×10⁻⁷). In some embodiments, a Cryptic protein:ALK6 heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a Cryptic protein:ALK6 heteromultimer of the disclosure has a different TGF-beta ligand binding and/or inhibition profile (specificity) compared to a corresponding homomultimer (e.g., Cryptic protein and ALK6 homomultimers). In some embodiments, a Cryptic protein:ALK6 heteromultimer of the disclosure is a heterodimer.

In certain aspects, the disclosure relates to heteromultimers that comprise at least one Cryptic protein polypeptide, which includes fragments, functional variants, and modified forms thereof, and at least one ALK7 polypeptide, which includes fragments, functional variants, and modified forms thereof. In some embodiments, the Cryptic protein:ALK7 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 521, 522, 525, 526, 529, or 530. In some embodiments, the Cryptic protein:ALK7 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-90 of SEQ ID NO: 521 and ends at any one of amino acids 157-233 of SEQ ID NO: 521. In some embodiments, the Cryptic protein:ALK7 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-30 of SEQ ID NO: 525 and ends at any one of amino acids 82-191 of SEQ ID NO: 525. In some embodiments, the Cryptic protein:ALK7 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-30 of SEQ ID NO: 529, and ends at any one of amino acids 82-148 of SEQ ID NO: 529. In some embodiments, the Cryptic protein:ALK7 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: SEQ ID NO: 38, 39, 301, 302, 305, 306, 309, 310, or 313. In some embodiments the Cryptic protein:ALK7 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 21-28 of SEQ ID NO: 38 and ends at any one of amino acids 92-113 of SEQ ID NO: 38. In some embodiments the Cryptic protein:ALK7 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-13 of SEQ ID NO: 301 and ends at any one of amino acids 42-63 of SEQ ID NO: 301. In some embodiments the Cryptic protein:ALK7 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 21-28 of SEQ ID NO: 305 and ends at any one of amino acids 411-413 of SEQ ID NO: 305. In some embodiments the Cryptic protein:ALK7 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 21-28 of SEQ ID NO: 309 and ends at any one of amino acids 334-336 of SEQ ID NO: 309. In certain preferred embodiments, Cryptic protein:ALK7 heteromultimers are soluble. In some embodiments, a Cryptic protein:ALK7 heteromultimer of the disclosure binds to one or more TGF-beta superfamily ligands (e.g., binds to one or more TGF-beta superfamily ligands with a K_{D} of at least 1×10⁻⁷). In some embodiments, a Cryptic protein:ALK7 heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a Cryptic protein:ALK7 heteromultimer of the disclosure has a different TGF-beta ligand binding and/or inhibition profile (specificity) compared to a corresponding homomultimer (e.g., Cryptic protein and ALK7 homomultimers). In some embodiments, a Cryptic protein:ALK7 heteromultimer of the disclosure is a heterodimer.

In certain aspects, the disclosure relates to heteromultimers that comprise at least one Cryptic family protein 1B polypeptide, which includes fragments, functional variants, and modified forms thereof, and at least one ALK1 polypeptide, which includes fragments, functional variants, and modified forms thereof. In some embodiments, the Cryptic family protein 1B:ALK1 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 533 or 534. In some embodiments, the Cryptic family protein 1B:ALK1 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-30 of SEQ ID NO: 533 and ends at any one of amino acids 82-223 of SEQ ID NO: 533. In some embodiments, the Cryptic family protein 1B:ALK1 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 14 or 15. In some embodiments, the Cryptic family protein 1B:ALK1 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 22-34 of SEQ ID NO: 14 and ends at any one of amino acids 95-118 of SEQ ID NO: 14. In certain preferred embodiments, Cryptic family protein 1B:ALK1 heteromultimers are soluble. In some embodiments, a Cryptic family protein 1B:ALK1 heteromultimer of the disclosure binds to one or more TGF-beta superfamily ligands (e.g., binds to one or more TGF-beta superfamily ligands with a K_{D} of at least 1×10⁻⁷). In some embodiments, a Cryptic family protein 1B:ALK1 heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a Cryptic family protein 1B:ALK1 heteromultimer of the disclosure has a different TGF-beta ligand binding and/or inhibition profile (specificity) compared to a corresponding homomultimer (e.g., Cryptic family protein 1B and ALK1 homomultimers). In some embodiments, a Cryptic family protein 1B:ALK1 heteromultimer of the disclosure is a heterodimer.

In certain aspects, the disclosure relates to heteromultimers that comprise at least one Cryptic family protein 1B polypeptide, which includes fragments, functional variants, and modified forms thereof, and at least one ALK2 polypeptide, which includes fragments, functional variants, and modified forms thereof. In some embodiments, the Cryptic family protein 1B:ALK2 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 533 or 534. In some embodiments, the Cryptic family protein 1B:ALK2 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-30 of SEQ ID NO: 533 and ends at any one of amino acids 82-223 of SEQ ID NO: 533. In some embodiments, the Cryptic family protein 1B:ALK2 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: SEQ ID NO: 18 or 19. In some embodiments the Cryptic family protein 1B:ALK2 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 21-35 of SEQ ID NO: 18 and ends at any one of amino acids 99-123 of SEQ ID NO: 18. In certain preferred embodiments, Cryptic family protein 1B:ALK2 heteromultimers are soluble. In some embodiments, a Cryptic family protein 1B:ALK2 heteromultimer of the disclosure binds to one or more TGF-beta superfamily ligands (e.g., binds to one or more TGF-beta superfamily ligands with a K_{D} of at least 1×10⁻⁷). In some embodiments, a Cryptic family protein 1B:ALK2 heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a Cryptic family protein 1B:ALK2 heteromultimer of the disclosure has a different TGF-beta ligand binding and/or inhibition profile (specificity) compared to a corresponding homomultimer (e.g., Cryptic family protein 1B and ALK2 homomultimers). In some embodiments, a Cryptic family protein 1B:ALK2 heteromultimer of the disclosure is a heterodimer.

In certain aspects, the disclosure relates to heteromultimers that comprise at least one Cryptic family protein 1B polypeptide, which includes fragments, functional variants, and modified forms thereof, and at least one ALK3 polypeptide, which includes fragments, functional variants, and modified forms thereof. In some embodiments, the Cryptic family protein 1B:ALK3 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 533 or 534. In some embodiments, the Cryptic family protein 1B:ALK3 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-30 of SEQ ID NO: 533 and ends at any one of amino acids 82-223 of SEQ ID NO: 533. In some embodiments, the Cryptic family protein 1B:ALK3 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: SEQ ID NO: 22 or 23. In some embodiments the Cryptic family protein 1B:ALK3 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 24-61 of SEQ ID NO: 22 and ends at any one of amino acids 130-152 of SEQ ID NO: 22. In certain preferred embodiments, Cryptic family protein 1B:ALK3 heteromultimers are soluble. In some embodiments, a Cryptic family protein 1B:ALK3 heteromultimer of the disclosure binds to one or more TGF-beta superfamily ligands (e.g., binds to one or more TGF-beta superfamily ligands with a K_{D} of at least 1×10⁻⁷). In some embodiments, a Cryptic family protein 1B:ALK3 heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a Cryptic family protein 1B:ALK3 heteromultimer of the disclosure has a different TGF-beta ligand binding and/or inhibition profile (specificity) compared to a corresponding homomultimer (e.g., Cryptic family protein 1B and ALK3 homomultimers). In some embodiments, a Cryptic family protein 1B:ALK3 heteromultimer of the disclosure is a heterodimer.

In certain aspects, the disclosure relates to heteromultimers that comprise at least one Cryptic family protein 1B polypeptide, which includes fragments, functional variants, and modified forms thereof, and at least one ALK4 polypeptide, which includes fragments, functional variants, and modified forms thereof. In some embodiments, the Cryptic family protein 1B:ALK4 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 533 or 534. In some embodiments, the Cryptic family protein 1B:ALK4 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-30 of SEQ ID NO: 533 and ends at any one of amino acids 82-223 of SEQ ID NO: 533. In some embodiments, the Cryptic family protein 1B:ALK4 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: SEQ ID NO: 26, 27, 83, or 84. In some embodiments the Cryptic family protein 1B:ALK4 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 24-34 of SEQ ID NO: 26 and ends at any one of amino acids 101-126 of SEQ ID NO: 26. In some embodiments the Cryptic family protein 1B:ALK4 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 24-34 of SEQ ID NO: 83 and ends at any one of amino acids 101-126 of SEQ ID NO: 83. In certain preferred embodiments, Cryptic family protein 1B:ALK4 heteromultimers are soluble. In some embodiments, a Cryptic family protein 1B:ALK4 heteromultimer of the disclosure binds to one or more TGF-beta superfamily ligands (e.g., binds to one or more TGF-beta superfamily ligands with a K_{D} of at least 1×10⁻⁷). In some embodiments, a Cryptic family protein 1B:ALK4 heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a Cryptic family protein 1B:ALK4 heteromultimer of the disclosure has a different TGF-beta ligand binding and/or inhibition profile (specificity) compared to a corresponding homomultimer (e.g., Cryptic family protein 1B and ALK4 homomultimers). In some embodiments, a Cryptic family protein 1B:ALK4 heteromultimer of the disclosure is a heterodimer.

In certain aspects, the disclosure relates to heteromultimers that comprise at least one Cryptic family protein 1B polypeptide, which includes fragments, functional variants, and modified forms thereof, and at least one ALK5 polypeptide, which includes fragments, functional variants, and modified forms thereof. In some embodiments, the Cryptic family protein 1B:ALK5 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 533 or 534. In some embodiments, the Cryptic family protein 1B:ALK5 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-30 of SEQ ID NO: 533 and ends at any one of amino acids 82-223 of SEQ ID NO: 533. In some embodiments, the Cryptic family protein 1B:ALK5 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: SEQ ID NO: 30, 31, 87, or 88. In some embodiments the Cryptic family protein 1B:ALK5 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 25-36 of SEQ ID NO: 30 and ends at any one of amino acids 101-126 of SEQ ID NO: 30. In some embodiments the Cryptic family protein 1B:ALK5 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 25-36 of SEQ ID NO: 87 and ends at any one of amino acids 101-130 of SEQ ID NO: 87. In certain preferred embodiments, Cryptic family protein 1B:ALK5 heteromultimers are soluble. In some embodiments, a Cryptic family protein 1B:ALK5 heteromultimer of the disclosure binds to one or more TGF-beta superfamily ligands (e.g., binds to one or more TGF-beta superfamily ligands with a K_{D} of at least 1×10⁻⁷). In some embodiments, a Cryptic family protein 1B:ALK5 heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a Cryptic family protein 1B:ALK5 heteromultimer of the disclosure has a different TGF-beta ligand binding and/or inhibition profile (specificity) compared to a corresponding homomultimer (e.g., Cryptic family protein 1B and ALK5 homomultimers). In some embodiments, a Cryptic family protein 1B:ALK5 heteromultimer of the disclosure is a heterodimer.

In certain aspects, the disclosure relates to heteromultimers that comprise at least one Cryptic family protein 1B polypeptide, which includes fragments, functional variants, and modified forms thereof, and at least one ALK6 polypeptide, which includes fragments, functional variants, and modified forms thereof. In some embodiments, the Cryptic family protein 1B:ALK6 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 533 or 534. In some embodiments, the Cryptic family protein 1B:ALK6 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-30 of SEQ ID NO: 533 and ends at any one of amino acids 82-223 of SEQ ID NO: 533. In some embodiments, the Cryptic family protein 1B:ALK6 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: SEQ ID NO: 34, 35, 91, or 92. In some embodiments the Cryptic family protein 1B:ALK6 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 14-32 of SEQ ID NO: 34 and ends at any one of amino acids 102-126 of SEQ ID NO: 34. In some embodiments the Cryptic family protein 1B:ALK6 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-62 of SEQ ID NO: 91 and ends at any one of amino acids 132-156 of SEQ ID NO: 91. In certain preferred embodiments, Cryptic family protein 1B:ALK6 heteromultimers are soluble. In some embodiments, a Cryptic family protein 1B:ALK6 heteromultimer of the disclosure binds to one or more TGF-beta superfamily ligands (e.g., binds to one or more TGF-beta superfamily ligands with a K_{D} of at least 1×10⁻⁷). In some embodiments, a Cryptic family protein 1B:ALK6 heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a Cryptic family protein 1B:ALK6 heteromultimer of the disclosure has a different TGF-beta ligand binding and/or inhibition profile (specificity) compared to a corresponding homomultimer (e.g., Cryptic family protein 1B and ALK6 homomultimers). In some embodiments, a Cryptic family protein 1B:ALK6 heteromultimer of the disclosure is a heterodimer.

In certain aspects, the disclosure relates to heteromultimers that comprise at least one Cryptic family protein 1B polypeptide, which includes fragments, functional variants, and modified forms thereof, and at least one ALK7 polypeptide, which includes fragments, functional variants, and modified forms thereof. In some embodiments, the Cryptic family protein 1B:ALK7 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 533 or 534. In some embodiments, the Cryptic family protein 1B:ALK7 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-30 of SEQ ID NO: 533 and ends at any one of amino acids 82-223 of SEQ ID NO: 533. In some embodiments, the Cryptic family protein 1B:ALK7 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: SEQ ID NO: 38, 39, 301, 302, 305, 306, 309, 310, or 313. In some embodiments the Cryptic family protein 1B:ALK7 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 21-28 of SEQ ID NO: 38 and ends at any one of amino acids 92-113 of SEQ ID NO: 38. In some embodiments the Cryptic family protein 1B:ALK7 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-13 of SEQ ID NO: 301 and ends at any one of amino acids 42-63 of SEQ ID NO: 301. In some embodiments the Cryptic family protein 1B:ALK7 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 21-28 of SEQ ID NO: 305 and ends at any one of amino acids 411-413 of SEQ ID NO: 305. In some embodiments the Cryptic family protein 1B:ALK7 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 21-28 of SEQ ID NO: 309 and ends at any one of amino acids 334-336 of SEQ ID NO: 309. In certain preferred embodiments, Cryptic family protein 1B:ALK7 heteromultimers are soluble. In some embodiments, a Cryptic family protein 1B:ALK7 heteromultimer of the disclosure binds to one or more TGF-beta superfamily ligands (e.g., binds to one or more TGF-beta superfamily ligands with a K_{D} of at least 1×10⁻⁷). In some embodiments, a Cryptic family protein 1B:ALK7 heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a Cryptic family protein 1B:ALK7 heteromultimer of the disclosure has a different TGF-beta ligand binding and/or inhibition profile (specificity) compared to a corresponding homomultimer (e.g., Cryptic family protein 1B and ALK7 homomultimers). In some embodiments, a Cryptic family protein 1B:ALK7 heteromultimer of the disclosure is a heterodimer.

In certain aspects, the disclosure relates to heteromultimers that comprise at least one Crim1 polypeptide, which includes fragments, functional variants, and modified forms thereof, and at least one ALK1 polypeptide, which includes fragments, functional variants, and modified forms thereof. In some embodiments, the Crim1:ALK1 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 537 or 538. In some embodiments, the Crim1:ALK1 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 35-37 of SEQ ID NO: 537 and ends at any one of amino acids 873-939 of SEQ ID NO: 537. In some embodiments, the Crim1:ALK1 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 537 or 538. In some embodiments, the Crim1:ALK1 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 35-37 of SEQ ID NO: 537 and ends at any one of amino acids 873-939 of SEQ ID NO: 537. In some embodiments, the Crim1:ALK1 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 14 or 15. In some embodiments, the Crim1:ALK1 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 22-34 of SEQ ID NO: 14 and ends at any one of amino acids 95-118 of SEQ ID NO: 14. In certain preferred embodiments, Crim1:ALK1 heteromultimers are soluble. In some embodiments, a Crim1:ALK1 heteromultimer of the disclosure binds to one or more TGF-beta superfamily ligands (e.g., binds to one or more TGF-beta superfamily ligands with a K_{D} of at least 1×10⁻⁷). In some embodiments, a Crim1:ALK1 heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a Crim1:ALK1 heteromultimer of the disclosure has a different TGF-beta ligand binding and/or inhibition profile (specificity) compared to a corresponding homomultimer (e.g., Crim1 and ALK1 homomultimers). In some embodiments, a Crim1:ALK1 heteromultimer of the disclosure is a heterodimer.

In certain aspects, the disclosure relates to heteromultimers that comprise at least one Crim1 polypeptide, which includes fragments, functional variants, and modified forms thereof, and at least one ALK2 polypeptide, which includes fragments, functional variants, and modified forms thereof. In some embodiments, the Crim1:ALK2 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 537 or 538. In some embodiments, the Crim1:ALK2 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 35-37 of SEQ ID NO: 537 and ends at any one of amino acids 873-939 of SEQ ID NO: 537. In some embodiments, the Crim1:ALK2 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: SEQ ID NO: 18 or 19. In some embodiments the Crim1:ALK2 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 21-35 of SEQ ID NO: 18 and ends at any one of amino acids 99-123 of SEQ ID NO: 18. In certain preferred embodiments, Crim1:ALK2 heteromultimers are soluble. In some embodiments, a Crim1:ALK2 heteromultimer of the disclosure binds to one or more TGF-beta superfamily ligands (e.g., binds to one or more TGF-beta superfamily ligands with a K_{D} of at least 1×10⁻⁷). In some embodiments, a Crim1:ALK2 heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a Crim1:ALK2 heteromultimer of the disclosure has a different TGF-beta ligand binding and/or inhibition profile (specificity) compared to a corresponding homomultimer (e.g., Crim1 and ALK2 homomultimers). In some embodiments, a Crim1:ALK2 heteromultimer of the disclosure is a heterodimer.

In certain aspects, the disclosure relates to heteromultimers that comprise at least one Crim1 polypeptide, which includes fragments, functional variants, and modified forms thereof, and at least one ALK3 polypeptide, which includes fragments, functional variants, and modified forms thereof. In some embodiments, the Crim1:ALK3 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 537 or 538. In some embodiments, the Crim1:ALK3 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 35-37 of SEQ ID NO: 537 and ends at any one of amino acids 873-939 of SEQ ID NO: 537. In some embodiments, the Crim1:ALK3 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: SEQ ID NO: 22 or 23. In some embodiments the Crim1:ALK3 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 24-61 of SEQ ID NO: 22 and ends at any one of amino acids 130-152 of SEQ ID NO: 22. In certain preferred embodiments, Crim1:ALK3 heteromultimers are soluble. In some embodiments, a Crim1:ALK3 heteromultimer of the disclosure binds to one or more TGF-beta superfamily ligands (e.g., binds to one or more TGF-beta superfamily ligands with a K_{D} of at least 1×10⁻⁷). In some embodiments, a Crim1:ALK3 heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a Crim1:ALK3 heteromultimer of the disclosure has a different TGF-beta ligand binding and/or inhibition profile (specificity) compared to a corresponding homomultimer (e.g., Crim1 and ALK3 homomultimers). In some embodiments, a Crim1:ALK3 heteromultimer of the disclosure is a heterodimer.

In certain aspects, the disclosure relates to heteromultimers that comprise at least one Crim1 polypeptide, which includes fragments, functional variants, and modified forms thereof, and at least one ALK4 polypeptide, which includes fragments, functional variants, and modified forms thereof. In some embodiments, the Crim1:ALK4 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 537 or 538. In some embodiments, the Crim1:ALK4 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 35-37 of SEQ ID NO: 537 and ends at any one of amino acids 873-939 of SEQ ID NO: 537. In some embodiments, the Crim1:ALK4 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: SEQ ID NO: 26, 27, 83, or 84. In some embodiments the Crim1:ALK4 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 24-34 of SEQ ID NO: 26 and ends at any one of amino acids 101-126 of SEQ ID NO: 26. In some embodiments the Crim1:ALK4 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 24-34 of SEQ ID NO: 83 and ends at any one of amino acids 101-126 of SEQ ID NO: 83. In certain preferred embodiments, Crim1:ALK4 heteromultimers are soluble. In some embodiments, a Crim1:ALK4 heteromultimer of the disclosure binds to one or more TGF-beta superfamily ligands (e.g., binds to one or more TGF-beta superfamily ligands with a K_{D} of at least 1×10⁻⁷). In some embodiments, a Crim1:ALK4 heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a Crim1:ALK4 heteromultimer of the disclosure has a different TGF-beta ligand binding and/or inhibition profile (specificity) compared to a corresponding homomultimer (e.g., Crim1 and ALK4 homomultimers). In some embodiments, a Crim1:ALK4 heteromultimer of the disclosure is a heterodimer.

In certain aspects, the disclosure relates to heteromultimers that comprise at least one Crim1 polypeptide, which includes fragments, functional variants, and modified forms thereof, and at least one ALK5 polypeptide, which includes fragments, functional variants, and modified forms thereof. In some embodiments, the Crim1:ALK5 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 537 or 538. In some embodiments, the Crim1:ALK5 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 35-37 of SEQ ID NO: 537 and ends at any one of amino acids 873-939 of SEQ ID NO: 537. In some embodiments, the Crim1:ALK5 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: SEQ ID NO: 30, 31, 87, or 88. In some embodiments the Crim1:ALK5 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 25-36 of SEQ ID NO: 30 and ends at any one of amino acids 101-126 of SEQ ID NO: 30. In some embodiments the Crim1:ALK5 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 25-36 of SEQ ID NO: 87 and ends at any one of amino acids 101-130 of SEQ ID NO: 87. In certain preferred embodiments, Crim1:ALK5 heteromultimers are soluble. In some embodiments, a Crim1:ALK5 heteromultimer of the disclosure binds to one or more TGF-beta superfamily ligands (e.g., binds to one or more TGF-beta superfamily ligands with a K_{D} of at least 1×10⁻⁷). In some embodiments, a Crim1:ALK5 heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a Crim1:ALK5 heteromultimer of the disclosure has a different TGF-beta ligand binding and/or inhibition profile (specificity) compared to a corresponding homomultimer (e.g., Crim1 and ALK5 homomultimers). In some embodiments, a Crim1:ALK5 heteromultimer of the disclosure is a heterodimer.

In certain aspects, the disclosure relates to heteromultimers that comprise at least one Crim1 polypeptide, which includes fragments, functional variants, and modified forms thereof, and at least one ALK6 polypeptide, which includes fragments, functional variants, and modified forms thereof. In some embodiments, the Crim1:ALK6 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 537 or 538. In some embodiments, the Crim1:ALK6 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 35-37 of SEQ ID NO: 537 and ends at any one of amino acids 873-939 of SEQ ID NO: 537. In some embodiments, the Crim1:ALK6 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: SEQ ID NO: 34, 35, 91, or 92. In some embodiments the Crim1:ALK6 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 14-32 of SEQ ID NO: 34 and ends at any one of amino acids 102-126 of SEQ ID NO: 34. In some embodiments the Crim1:ALK6 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-62 of SEQ ID NO: 91 and ends at any one of amino acids 132-156 of SEQ ID NO: 91. In certain preferred embodiments, Crim1:ALK6 heteromultimers are soluble. In some embodiments, a Crim1:ALK6 heteromultimer of the disclosure binds to one or more TGF-beta superfamily ligands (e.g., binds to one or more TGF-beta superfamily ligands with a K_{D} of at least 1×10⁻⁷). In some embodiments, a Crim1:ALK6 heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a Crim1:ALK6 heteromultimer of the disclosure has a different TGF-beta ligand binding and/or inhibition profile (specificity) compared to a corresponding homomultimer (e.g., Crim1 and ALK6 homomultimers). In some embodiments, a Crim1:ALK6 heteromultimer of the disclosure is a heterodimer.

In certain aspects, the disclosure relates to heteromultimers that comprise at least one Crim1 polypeptide, which includes fragments, functional variants, and modified forms thereof, and at least one ALK7 polypeptide, which includes fragments, functional variants, and modified forms thereof. In some embodiments, the Crim1:ALK7 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 537 or 538. In some embodiments, the Crim1:ALK7 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 35-37 of SEQ ID NO: 537 and ends at any one of amino acids 873-939 of SEQ ID NO: 537. In some embodiments, the Crim1:ALK7 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: SEQ ID NO: 38, 39, 301, 302, 305, 306, 309, 310, or 313. In some embodiments the Crim1:ALK7 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 21-28 of SEQ ID NO: 38 and ends at any one of amino acids 92-113 of SEQ ID NO: 38. In some embodiments the Crim1:ALK7 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-13 of SEQ ID NO: 301 and ends at any one of amino acids 42-63 of SEQ ID NO: 301. In some embodiments the Crim1:ALK7 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 21-28 of SEQ ID NO: 305 and ends at any one of amino acids 411-413 of SEQ ID NO: 305. In some embodiments the Crim1:ALK7 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 21-28 of SEQ ID NO: 309 and ends at any one of amino acids 334-336 of SEQ ID NO: 309. In certain preferred embodiments, Crim1:ALK7 heteromultimers are soluble. In some embodiments, a Crim1:ALK7 heteromultimer of the disclosure binds to one or more TGF-beta superfamily ligands (e.g., binds to one or more TGF-beta superfamily ligands with a K_{D} of at least 1×10⁻⁷). In some embodiments, a Crim1:ALK7 heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a Crim1:ALK7 heteromultimer of the disclosure has a different TGF-beta ligand binding and/or inhibition profile (specificity) compared to a corresponding homomultimer (e.g., Crim1 and ALK7 homomultimers). In some embodiments, a Crim1:ALK7 heteromultimer of the disclosure is a heterodimer.

In certain aspects, the disclosure relates to heteromultimers that comprise at least one Crim2 polypeptide, which includes fragments, functional variants, and modified forms thereof, and at least one ALK1 polypeptide, which includes fragments, functional variants, and modified forms thereof. In some embodiments, the Crim2:ALK1 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 541, 542, 545, or 546. In some embodiments, the Crim2:ALK1 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-138 of SEQ ID NO: 541 and ends at any one of amino acids 1298-1503 of SEQ ID NO: 541. In some embodiments, the Crim2:ALK1 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 24-138 of SEQ ID NO: 545 and ends at any one of amino acids 539-814 of SEQ ID NO: 545. In some embodiments, the Crim2:ALK1 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 14 or 15. In some embodiments, the Crim2:ALK1 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 22-34 of SEQ ID NO: 14 and ends at any one of amino acids 95-118 of SEQ ID NO: 14. In certain preferred embodiments, Crim2:ALK1 heteromultimers are soluble. In some embodiments, a Crim2:ALK1 heteromultimer of the disclosure binds to one or more TGF-beta superfamily ligands (e.g., binds to one or more TGF-beta superfamily ligands with a K_{D} of at least 1×10⁻⁷). In some embodiments, a Crim2:ALK1 heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a Crim2:ALK1 heteromultimer of the disclosure has a different TGF-beta ligand binding and/or inhibition profile (specificity) compared to a corresponding homomultimer (e.g., Crim2 and ALK1 homomultimers). In some embodiments, a Crim2:ALK1 heteromultimer of the disclosure is a heterodimer.

In certain aspects, the disclosure relates to heteromultimers that comprise at least one Crim2 polypeptide, which includes fragments, functional variants, and modified forms thereof, and at least one ALK2 polypeptide, which includes fragments, functional variants, and modified forms thereof. In some embodiments, the Crim2:ALK2 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 541, 542, 545, or 546. In some embodiments, the Crim2:ALK2 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-138 of SEQ ID NO: 541 and ends at any one of amino acids 1298-1503 of SEQ ID NO: 541. In some embodiments, the Crim2:ALK2 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 24-138 of SEQ ID NO: 545 and ends at any one of amino acids 539-814 of SEQ ID NO: 545. In some embodiments, the Crim2:ALK2 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: SEQ ID NO: 18 or 19. In some embodiments the Crim2:ALK2 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 21-35 of SEQ ID NO: 18 and ends at any one of amino acids 99-123 of SEQ ID NO: 18. In certain preferred embodiments, Crim2:ALK2 heteromultimers are soluble. In some embodiments, a Crim2:ALK2 heteromultimer of the disclosure binds to one or more TGF-beta superfamily ligands (e.g., binds to one or more TGF-beta superfamily ligands with a K_{D} of at least 1×10⁻⁷). In some embodiments, a Crim2:ALK2 heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a Crim2:ALK2 heteromultimer of the disclosure has a different TGF-beta ligand binding and/or inhibition profile (specificity) compared to a corresponding homomultimer (e.g., Crim2 and ALK2 homomultimers). In some embodiments, a Crim2:ALK2 heteromultimer of the disclosure is a heterodimer.

In certain aspects, the disclosure relates to heteromultimers that comprise at least one Crim2 polypeptide, which includes fragments, functional variants, and modified forms thereof, and at least one ALK3 polypeptide, which includes fragments, functional variants, and modified forms thereof. In some embodiments, the Crim2:ALK3 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 541, 542, 545, or 546. In some embodiments, the Crim2:ALK3 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-138 of SEQ ID NO: 541 and ends at any one of amino acids 1298-1503 of SEQ ID NO: 541. In some embodiments, the Crim2:ALK3 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 24-138 of SEQ ID NO: 545 and ends at any one of amino acids 539-814 of SEQ ID NO: 545. In some embodiments, the Crim2:ALK1 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 541, 542, 545, or 546. In some embodiments, the Crim2:ALK1 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-138 of SEQ ID NO: 541 and ends at any one of amino acids 1298-1503 of SEQ ID NO: 541. In some embodiments, the Crim2:ALK1 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 24-138 of SEQ ID NO: 545 and ends at any one of amino acids 539-814 of SEQ ID NO: 545. In certain preferred embodiments, Crim2:ALK3 heteromultimers are soluble. In some embodiments, a Crim2:ALK3 heteromultimer of the disclosure binds to one or more TGF-beta superfamily ligands (e.g., binds to one or more TGF-beta superfamily ligands with a K_{D} of at least 1×10⁻⁷). In some embodiments, a Crim2:ALK3 heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a Crim2:ALK3 heteromultimer of the disclosure has a different TGF-beta ligand binding and/or inhibition profile (specificity) compared to a corresponding homomultimer (e.g., Crim2 and ALK3 homomultimers). In some embodiments, a Crim2:ALK3 heteromultimer of the disclosure is a heterodimer.

In certain aspects, the disclosure relates to heteromultimers that comprise at least one Crim2 polypeptide, which includes fragments, functional variants, and modified forms thereof, and at least one ALK4 polypeptide, which includes fragments, functional variants, and modified forms thereof. In some embodiments, the Crim2:ALK4 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 541, 542, 545, or 546. In some embodiments, the Crim2:ALK4 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-138 of SEQ ID NO: 541 and ends at any one of amino acids 1298-1503 of SEQ ID NO: 541. In some embodiments, the Crim2:ALK4 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 24-138 of SEQ ID NO: 545 and ends at any one of amino acids 539-814 of SEQ ID NO: 545. In some embodiments, the Crim2:ALK4 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: SEQ ID NO: 26, 27, 83, or 84. In some embodiments the Crim2:ALK4 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 24-34 of SEQ ID NO: 26 and ends at any one of amino acids 101-126 of SEQ ID NO: 26. In some embodiments the Crim2:ALK4 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 24-34 of SEQ ID NO: 83 and ends at any one of amino acids 101-126 of SEQ ID NO: 83. In certain preferred embodiments, Crim2:ALK4 heteromultimers are soluble. In some embodiments, a Crim2:ALK4 heteromultimer of the disclosure binds to one or more TGF-beta superfamily ligands (e.g., binds to one or more TGF-beta superfamily ligands with a K_{D} of at least 1×10⁻⁷). In some embodiments, a Crim2:ALK4 heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a Crim2:ALK4 heteromultimer of the disclosure has a different TGF-beta ligand binding and/or inhibition profile (specificity) compared to a corresponding homomultimer (e.g., Crim2 and ALK4 homomultimers). In some embodiments, a Crim2:ALK4 heteromultimer of the disclosure is a heterodimer.

In certain aspects, the disclosure relates to heteromultimers that comprise at least one Crim2 polypeptide, which includes fragments, functional variants, and modified forms thereof, and at least one ALK5 polypeptide, which includes fragments, functional variants, and modified forms thereof. In some embodiments, the Crim2:ALK5 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 541, 542, 545, or 546. In some embodiments, the Crim2:ALK5 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-138 of SEQ ID NO: 541 and ends at any one of amino acids 1298-1503 of SEQ ID NO: 541. In some embodiments, the Crim2:ALK5 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 24-138 of SEQ ID NO: 545 and ends at any one of amino acids 539-814 of SEQ ID NO: 545. In some embodiments, the Crim2:ALK5 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: SEQ ID NO: 30, 31, 87, or 88. In some embodiments the Crim2:ALK5 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 25-36 of SEQ ID NO: 30 and ends at any one of amino acids 101-126 of SEQ ID NO: 30. In some embodiments the Crim2:ALK5 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 25-36 of SEQ ID NO: 87 and ends at any one of amino acids 101-130 of SEQ ID NO: 87. In certain preferred embodiments, Crim2:ALK5 heteromultimers are soluble. In some embodiments, a Crim2:ALK5 heteromultimer of the disclosure binds to one or more TGF-beta superfamily ligands (e.g., binds to one or more TGF-beta superfamily ligands with a K_{D} of at least 1×10⁻⁷). In some embodiments, a Crim2:ALK5 heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a Crim2:ALK5 heteromultimer of the disclosure has a different TGF-beta ligand binding and/or inhibition profile (specificity) compared to a corresponding homomultimer (e.g., Crim2 and ALK5 homomultimers). In some embodiments, a Crim2:ALK5 heteromultimer of the disclosure is a heterodimer.

In certain aspects, the disclosure relates to heteromultimers that comprise at least one Crim2 polypeptide, which includes fragments, functional variants, and modified forms thereof, and at least one ALK6 polypeptide, which includes fragments, functional variants, and modified forms thereof. In some embodiments, the Crim2:ALK6 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 541, 542, 545, or 546. In some embodiments, the Crim2:ALK6 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-138 of SEQ ID NO: 541 and ends at any one of amino acids 1298-1503 of SEQ ID NO: 541. In some embodiments, the Crim2:ALK6 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 24-138 of SEQ ID NO: 545 and ends at any one of amino acids 539-814 of SEQ ID NO: 545. In some embodiments, the Crim2:ALK6 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: SEQ ID NO: 34, 35, 91, or 92. In some embodiments the Crim2:ALK6 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 14-32 of SEQ ID NO: 34 and ends at any one of amino acids 102-126 of SEQ ID NO: 34. In some embodiments the Crim2:ALK6 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-62 of SEQ ID NO: 91 and ends at any one of amino acids 132-156 of SEQ ID NO: 91. In certain preferred embodiments, Crim2:ALK6 heteromultimers are soluble. In some embodiments, a Crim2:ALK6 heteromultimer of the disclosure binds to one or more TGF-beta superfamily ligands (e.g., binds to one or more TGF-beta superfamily ligands with a K_{D} of at least 1×10⁻⁷). In some embodiments, a Crim2:ALK6 heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a Crim2:ALK6 heteromultimer of the disclosure has a different TGF-beta ligand binding and/or inhibition profile (specificity) compared to a corresponding homomultimer (e.g., Crim2 and ALK6 homomultimers). In some embodiments, a Crim2:ALK6 heteromultimer of the disclosure is a heterodimer.

In certain aspects, the disclosure relates to heteromultimers that comprise at least one Crim2 polypeptide, which includes fragments, functional variants, and modified forms thereof, and at least one ALK7 polypeptide, which includes fragments, functional variants, and modified forms thereof. In some embodiments, the Crim2:ALK7 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 541, 542, 545, or 546. In some embodiments, the Crim2:ALK7 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-138 of SEQ ID NO: 541 and ends at any one of amino acids 1298-1503 of SEQ ID NO: 541. In some embodiments, the Crim2:ALK7 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 24-138 of SEQ ID NO: 545 and ends at any one of amino acids 539-814 of SEQ ID NO: 545. In some embodiments, the Crim2:ALK7 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: SEQ ID NO: 38, 39, 301, 302, 305, 306, 309, 310, or 313. In some embodiments the Crim2:ALK7 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 21-28 of SEQ ID NO: 38 and ends at any one of amino acids 92-113 of SEQ ID NO: 38. In some embodiments the Crim2:ALK7 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-13 of SEQ ID NO: 301 and ends at any one of amino acids 42-63 of SEQ ID NO: 301. In some embodiments the Crim2:ALK7 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 21-28 of SEQ ID NO: 305 and ends at any one of amino acids 411-413 of SEQ ID NO: 305. In some embodiments the Crim2:ALK7 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 21-28 of SEQ ID NO: 309 and ends at any one of amino acids 334-336 of SEQ ID NO: 309. In certain preferred embodiments, Crim2:ALK7 heteromultimers are soluble. In some embodiments, a Crim2:ALK7 heteromultimer of the disclosure binds to one or more TGF-beta superfamily ligands (e.g., binds to one or more TGF-beta superfamily ligands with a K_{D} of at least 1×10⁻⁷). In some embodiments, a Crim2:ALK7 heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a Crim2:ALK7 heteromultimer of the disclosure has a different TGF-beta ligand binding and/or inhibition profile (specificity) compared to a corresponding homomultimer (e.g., Crim2 and ALK7 homomultimers). In some embodiments, a Crim2:ALK7 heteromultimer of the disclosure is a heterodimer.

In certain aspects, the disclosure relates to heteromultimers that comprise at least one BAMBI polypeptide, which includes fragments, functional variants, and modified forms thereof, and at least one ALK1 polypeptide, which includes fragments, functional variants, and modified forms thereof. In some embodiments, the BAMBI:ALK1 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 549 or 550. In some embodiments, the BAMBI:ALK1 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 21-30 of SEQ ID NO: 549 and ends at any one of amino acids 104-152 of SEQ ID NO: 549. In some embodiments, the BAMBI:ALK1 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 14 or 15. In some embodiments, the BAMBI:ALK1 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 22-34 of SEQ ID NO: 14 and ends at any one of amino acids 95-118 of SEQ ID NO: 14. In certain preferred embodiments, BAMBI:ALK1 heteromultimers are soluble. In some embodiments, a BAMBI:ALK1 heteromultimer of the disclosure binds to one or more TGF-beta superfamily ligands (e.g., binds to one or more TGF-beta superfamily ligands with a K_{D} of at least 1×10⁻⁷). In some embodiments, a BAMBI:ALK1 heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a BAMBI:ALK1 heteromultimer of the disclosure has a different TGF-beta ligand binding and/or inhibition profile (specificity) compared to a corresponding homomultimer (e.g., BAMBI and ALK1 homomultimers). In some embodiments, a BAMBI:ALK1 heteromultimer of the disclosure is a heterodimer.

In certain aspects, the disclosure relates to heteromultimers that comprise at least one BAMBI polypeptide, which includes fragments, functional variants, and modified forms thereof, and at least one ALK2 polypeptide, which includes fragments, functional variants, and modified forms thereof. In some embodiments, the BAMBI:ALK2 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 549 or 550. In some embodiments, the BAMBI:ALK2 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 21-30 of SEQ ID NO: 549 and ends at any one of amino acids 104-152 of SEQ ID NO: 549. In some embodiments, the BAMBI:ALK2 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: SEQ ID NO: 18 or 19. In some embodiments the BAMBI:ALK2 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 21-35 of SEQ ID NO: 18 and ends at any one of amino acids 99-123 of SEQ ID NO: 18. In certain preferred embodiments, BAMBI:ALK2 heteromultimers are soluble. In some embodiments, a BAMBI:ALK2 heteromultimer of the disclosure binds to one or more TGF-beta superfamily ligands (e.g., binds to one or more TGF-beta superfamily ligands with a K_{D} of at least 1×10⁻⁷). In some embodiments, a BAMBI:ALK2 heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a BAMBI:ALK2 heteromultimer of the disclosure has a different TGF-beta ligand binding and/or inhibition profile (specificity) compared to a corresponding homomultimer (e.g., BAMBI and ALK2 homomultimers). In some embodiments, a BAMBI:ALK2 heteromultimer of the disclosure is a heterodimer.

In certain aspects, the disclosure relates to heteromultimers that comprise at least one BAMBI polypeptide, which includes fragments, functional variants, and modified forms thereof, and at least one ALK3 polypeptide, which includes fragments, functional variants, and modified forms thereof. In some embodiments, the BAMBI:ALK3 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 549 or 550. In some embodiments, the BAMBI:ALK3 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 21-30 of SEQ ID NO: 549 and ends at any one of amino acids 104-152 of SEQ ID NO: 549. In some embodiments, the BAMBI:ALK3 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: SEQ ID NO: 22 or 23. In some embodiments the BAMBI:ALK3 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 24-61 of SEQ ID NO: 22 and ends at any one of amino acids 130-152 of SEQ ID NO: 22. In certain preferred embodiments, BAMBI:ALK3 heteromultimers are soluble. In some embodiments, a BAMBI:ALK3 heteromultimer of the disclosure binds to one or more TGF-beta superfamily ligands (e.g., binds to one or more TGF-beta superfamily ligands with a K_{D} of at least 1×10⁻⁷). In some embodiments, a BAMBI:ALK3 heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a BAMBI:ALK3 heteromultimer of the disclosure has a different TGF-beta ligand binding and/or inhibition profile (specificity) compared to a corresponding homomultimer (e.g., BAMBI and ALK3 homomultimers). In some embodiments, a BAMBI:ALK3 heteromultimer of the disclosure is a heterodimer.

In certain aspects, the disclosure relates to heteromultimers that comprise at least one BAMBI polypeptide, which includes fragments, functional variants, and modified forms thereof, and at least one ALK4 polypeptide, which includes fragments, functional variants, and modified forms thereof. In some embodiments, the BAMBI:ALK4 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 549 or 550. In some embodiments, the BAMBI:ALK4 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 21-30 of SEQ ID NO: 549 and ends at any one of amino acids 104-152 of SEQ ID NO: 549. In some embodiments, the BAMBI:ALK4 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: SEQ ID NO: 26, 27, 83, or 84. In some embodiments the BAMBI:ALK4 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 24-34 of SEQ ID NO: 26 and ends at any one of amino acids 101-126 of SEQ ID NO: 26. In some embodiments the BAMBI:ALK4 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 24-34 of SEQ ID NO: 83 and ends at any one of amino acids 101-126 of SEQ ID NO: 83. In certain preferred embodiments, BAMBI:ALK4 heteromultimers are soluble. In some embodiments, a BAMBI:ALK4 heteromultimer of the disclosure binds to one or more TGF-beta superfamily ligands (e.g., binds to one or more TGF-beta superfamily ligands with a K_{D} of at least 1×10⁻⁷). In some embodiments, a BAMBI:ALK4 heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a BAMBI:ALK4 heteromultimer of the disclosure has a different TGF-beta ligand binding and/or inhibition profile (specificity) compared to a corresponding homomultimer (e.g., BAMBI and ALK4 homomultimers). In some embodiments, a BAMBI:ALK4 heteromultimer of the disclosure is a heterodimer.

In certain aspects, the disclosure relates to heteromultimers that comprise at least one BAMBI polypeptide, which includes fragments, functional variants, and modified forms thereof, and at least one ALK5 polypeptide, which includes fragments, functional variants, and modified forms thereof. In some embodiments, the BAMBI:ALK5 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 549 or 550. In some embodiments, the BAMBI:ALK5 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 21-30 of SEQ ID NO: 549 and ends at any one of amino acids 104-152 of SEQ ID NO: 549. In some embodiments, the BAMBI:ALK5 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: SEQ ID NO: 30, 31, 87, or 88. In some embodiments the BAMBI:ALK5 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 25-36 of SEQ ID NO: 30 and ends at any one of amino acids 101-126 of SEQ ID NO: 30. In some embodiments the BAMBI:ALK5 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 25-36 of SEQ ID NO: 87 and ends at any one of amino acids 101-130 of SEQ ID NO: 87. In certain preferred embodiments, BAMBI:ALK5 heteromultimers are soluble. In some embodiments, a BAMBI:ALK5 heteromultimer of the disclosure binds to one or more TGF-beta superfamily ligands (e.g., binds to one or more TGF-beta superfamily ligands with a K_{D} of at least 1×10⁻⁷). In some embodiments, a BAMBI:ALK5 heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a BAMBI:ALK5 heteromultimer of the disclosure has a different TGF-beta ligand binding and/or inhibition profile (specificity) compared to a corresponding homomultimer (e.g., BAMBI and ALK5 homomultimers). In some embodiments, a BAMBI:ALK5 heteromultimer of the disclosure is a heterodimer.

In certain aspects, the disclosure relates to heteromultimers that comprise at least one BAMBI polypeptide, which includes fragments, functional variants, and modified forms thereof, and at least one ALK6 polypeptide, which includes fragments, functional variants, and modified forms thereof. In some embodiments, the BAMBI:ALK6 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 549 or 550. In some embodiments, the BAMBI:ALK6 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 21-30 of SEQ ID NO: 549 and ends at any one of amino acids 104-152 of SEQ ID NO: 549. In some embodiments, the BAMBI:ALK6 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: SEQ ID NO: 34, 35, 91, or 92. In some embodiments the BAMBI:ALK6 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 14-32 of SEQ ID NO: 34 and ends at any one of amino acids 102-126 of SEQ ID NO: 34. In some embodiments the BAMBI:ALK6 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-62 of SEQ ID NO: 91 and ends at any one of amino acids 132-156 of SEQ ID NO: 91. In certain preferred embodiments, BAMBI:ALK6 heteromultimers are soluble. In some embodiments, a BAMBI:ALK6 heteromultimer of the disclosure binds to one or more TGF-beta superfamily ligands (e.g., binds to one or more TGF-beta superfamily ligands with a K_{D} of at least 1×10⁻⁷). In some embodiments, a BAMBI:ALK6 heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a BAMBI:ALK6 heteromultimer of the disclosure has a different TGF-beta ligand binding and/or inhibition profile (specificity) compared to a corresponding homomultimer (e.g., BAMBI and ALK6 homomultimers). In some embodiments, a BAMBI:ALK6 heteromultimer of the disclosure is a heterodimer.

In certain aspects, the disclosure relates to heteromultimers that comprise at least one BAMBI polypeptide, which includes fragments, functional variants, and modified forms thereof, and at least one ALK7 polypeptide, which includes fragments, functional variants, and modified forms thereof. In some embodiments, the BAMBI:ALK7 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 549 or 550. In some embodiments, the BAMBI:ALK7 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 21-30 of SEQ ID NO: 549 and ends at any one of amino acids 104-152 of SEQ ID NO: 549. In some embodiments, the BAMBI:ALK7 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: SEQ ID NO: 38, 39, 301, 302, 305, 306, 309, 310, or 313. In some embodiments the BAMBI:ALK7 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 21-28 of SEQ ID NO: 38 and ends at any one of amino acids 92-113 of SEQ ID NO: 38. In some embodiments the BAMBI:ALK7 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-13 of SEQ ID NO: 301 and ends at any one of amino acids 42-63 of SEQ ID NO: 301. In some embodiments the BAMBI:ALK7 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 21-28 of SEQ ID NO: 305 and ends at any one of amino acids 411-413 of SEQ ID NO: 305. In some embodiments the BAMBI:ALK7 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 21-28 of SEQ ID NO: 309 and ends at any one of amino acids 334-336 of SEQ ID NO: 309. In certain preferred embodiments, BAMBI:ALK7 heteromultimers are soluble. In some embodiments, a BAMBI:ALK7 heteromultimer of the disclosure binds to one or more TGF-beta superfamily ligands (e.g., binds to one or more TGF-beta superfamily ligands with a K_{D} of at least 1×10⁻⁷). In some embodiments, a BAMBI:ALK7 heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a BAMBI:ALK7 heteromultimer of the disclosure has a different TGF-beta ligand binding and/or inhibition profile (specificity) compared to a corresponding homomultimer (e.g., BAMBI and ALK7 homomultimers). In some embodiments, a BAMBI:ALK7 heteromultimer of the disclosure is a heterodimer.

In certain aspects, the disclosure relates to heteromultimers that comprise at least one BMPER polypeptide, which includes fragments, functional variants, and modified forms thereof, and at least one ALK1 polypeptide, which includes fragments, functional variants, and modified forms thereof. In some embodiments, the BMPER:ALK1 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 553 or 554. In some embodiments, the BMPER:ALK1 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 39-50 of SEQ ID NO: 553 and ends at any one of amino acids 364-369 of SEQ ID NO: 553. In some embodiments, the BMPER:ALK1 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 370-386 of SEQ ID NO: 553 and ends at any one of amino acids 682-685 of SEQ ID NO: 553. In some embodiments, the BMPER:ALK1 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 39-50 of SEQ ID NO: 553 and ends at any one of amino acids 682-685 of SEQ ID NO: 553. In some embodiments, the BMPER:ALK1 heteromultimer comprises a BMPER protein, wherein the BMPER protein is a dimer comprising a first polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 39-50 of SEQ ID NO: 553 and ends at any one of amino acids 364-369 of SEQ ID NO: 553, and second polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 370-386 of SEQ ID NO: 553, and ends at any one of amino acids 682-685 of SEQ ID NO: 553. In some embodiments, the BMPER:ALK1 heteromultimer comprises a single chain ligand trap that comprises a first BMPER polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 39-50 of SEQ ID NO: 553 and ends at any one of amino acids 364-369 of SEQ ID NO: 553, and second BMPER polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 370-386 of SEQ ID NO: 553 and ends at any one of amino acids 682-685 of SEQ ID NO: 553. In some embodiments, the BMPER:ALK1 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 14 or 15. In some embodiments, the BMPER:ALK1 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 22-34 of SEQ ID NO: 14 and ends at any one of amino acids 95-118 of SEQ ID NO: 14. In certain preferred embodiments, BMPER:ALK1 heteromultimers are soluble. In some embodiments, a BMPER:ALK1 heteromultimer of the disclosure binds to one or more TGF-beta superfamily ligands (e.g., binds to one or more TGF-beta superfamily ligands with a K_{D} of at least 1×10⁻⁷). In some embodiments, a BMPER:ALK1 heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a BMPER:ALK1 heteromultimer of the disclosure has a different TGF-beta ligand binding and/or inhibition profile (specificity) compared to a corresponding homomultimer (e.g., BMPER and ALK1 homomultimers). In some embodiments, a BMPER:ALK1 heteromultimer of the disclosure is a heterodimer.

In certain aspects, the disclosure relates to heteromultimers that comprise at least one BMPER polypeptide, which includes fragments, functional variants, and modified forms thereof, and at least one ALK2 polypeptide, which includes fragments, functional variants, and modified forms thereof. In some embodiments, the BMPER:ALK2 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 553 or 554. In some embodiments, the BMPER:ALK2 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 39-50 of SEQ ID NO: 553 and ends at any one of amino acids 364-369 of SEQ ID NO: 553. In some embodiments, the BMPER:ALK2 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 370-386 of SEQ ID NO: 553 and ends at any one of amino acids 682-685 of SEQ ID NO: 553. In some embodiments, the BMPER:ALK2 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 39-50 of SEQ ID NO: 553 and ends at any one of amino acids 682-685 of SEQ ID NO: 553. In some embodiments, the BMPER:ALK2 heteromultimer comprises a BMPER protein, wherein the BMPER protein is a dimer comprising a first polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 39-50 of SEQ ID NO: 553 and ends at any one of amino acids 364-369 of SEQ ID NO: 553, and second polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 370-386 of SEQ ID NO: 553, and ends at any one of amino acids 682-685 of SEQ ID NO: 553. In some embodiments, the BMPER:ALK2 heteromultimer comprises a single chain ligand trap that comprises a first BMPER polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 39-50 of SEQ ID NO: 553 and ends at any one of amino acids 364-369 of SEQ ID NO: 553, and second BMPER polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 370-386 of SEQ ID NO: 553 and ends at any one of amino acids 682-685 of SEQ ID NO: 553. In some embodiments, the BMPER:ALK2 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: SEQ ID NO: 18 or 19. In some embodiments the BMPER:ALK2 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 21-35 of SEQ ID NO: 18 and ends at any one of amino acids 99-123 of SEQ ID NO: 18. In certain preferred embodiments, BMPER:ALK2 heteromultimers are soluble. In some embodiments, a BMPER:ALK2 heteromultimer of the disclosure binds to one or more TGF-beta superfamily ligands (e.g., binds to one or more TGF-beta superfamily ligands with a K_{D} of at least 1×10⁻⁷). In some embodiments, a BMPER:ALK2 heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a BMPER:ALK2 heteromultimer of the disclosure has a different TGF-beta ligand binding and/or inhibition profile (specificity) compared to a corresponding homomultimer (e.g., BMPER and ALK2 homomultimers). In some embodiments, a BMPER:ALK2 heteromultimer of the disclosure is a heterodimer.

In certain aspects, the disclosure relates to heteromultimers that comprise at least one BMPER polypeptide, which includes fragments, functional variants, and modified forms thereof, and at least one ALK3 polypeptide, which includes fragments, functional variants, and modified forms thereof. In some embodiments, the BMPER:ALK3 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 553 or 554. In some embodiments, the BMPER:ALK3 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 39-50 of SEQ ID NO: 553 and ends at any one of amino acids 364-369 of SEQ ID NO: 553. In some embodiments, the BMPER:ALK3 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 370-386 of SEQ ID NO: 553 and ends at any one of amino acids 682-685 of SEQ ID NO: 553. In some embodiments, the BMPER:ALK3 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 39-50 of SEQ ID NO: 553 and ends at any one of amino acids 682-685 of SEQ ID NO: 553. In some embodiments, the BMPER:ALK3 heteromultimer comprises a BMPER protein, wherein the BMPER protein is a dimer comprising a first polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 39-50 of SEQ ID NO: 553 and ends at any one of amino acids 364-369 of SEQ ID NO: 553, and second polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 370-386 of SEQ ID NO: 553, and ends at any one of amino acids 682-685 of SEQ ID NO: 553. In some embodiments, the BMPER:ALK3 heteromultimer comprises a single chain ligand trap that comprises a first BMPER polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 39-50 of SEQ ID NO: 553 and ends at any one of amino acids 364-369 of SEQ ID NO: 553, and second BMPER polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 370-386 of SEQ ID NO: 553 and ends at any one of amino acids 682-685 of SEQ ID NO: 553. In some embodiments, the BMPER:ALK3 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: SEQ ID NO: 22 or 23. In some embodiments the BMPER:ALK3 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 24-61 of SEQ ID NO: 22 and ends at any one of amino acids 130-152 of SEQ ID NO: 22. In certain preferred embodiments, BMPER:ALK3 heteromultimers are soluble. In some embodiments, a BMPER:ALK3 heteromultimer of the disclosure binds to one or more TGF-beta superfamily ligands (e.g., binds to one or more TGF-beta superfamily ligands with a K_{D} of at least 1×10⁻⁷). In some embodiments, a BMPER:ALK3 heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a BMPER:ALK3 heteromultimer of the disclosure has a different TGF-beta ligand binding and/or inhibition profile (specificity) compared to a corresponding homomultimer (e.g., BMPER and ALK3 homomultimers). In some embodiments, a BMPER:ALK3 heteromultimer of the disclosure is a heterodimer.

In certain aspects, the disclosure relates to heteromultimers that comprise at least one BMPER polypeptide, which includes fragments, functional variants, and modified forms thereof, and at least one ALK4 polypeptide, which includes fragments, functional variants, and modified forms thereof. In some embodiments, the BMPER:ALK4 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 553 or 554. In some embodiments, the BMPER:ALK4 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 39-50 of SEQ ID NO: 553 and ends at any one of amino acids 364-369 of SEQ ID NO: 553. In some embodiments, the BMPER:ALK4 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 370-386 of SEQ ID NO: 553 and ends at any one of amino acids 682-685 of SEQ ID NO: 553. In some embodiments, the BMPER:ALK4 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 39-50 of SEQ ID NO: 553 and ends at any one of amino acids 682-685 of SEQ ID NO: 553. In some embodiments, the BMPER:ALK4 heteromultimer comprises a BMPER protein, wherein the BMPER protein is a dimer comprising a first polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 39-50 of SEQ ID NO: 553 and ends at any one of amino acids 364-369 of SEQ ID NO: 553, and second polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 370-386 of SEQ ID NO: 553, and ends at any one of amino acids 682-685 of SEQ ID NO: 553. In some embodiments, the BMPER:ALK4 heteromultimer comprises a single chain ligand trap that comprises a first BMPER polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 39-50 of SEQ ID NO: 553 and ends at any one of amino acids 364-369 of SEQ ID NO: 553, and second BMPER polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 370-386 of SEQ ID NO: 553 and ends at any one of amino acids 682-685 of SEQ ID NO: 553. In some embodiments, the BMPER:ALK4 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: SEQ ID NO: 26, 27, 83, or 84. In some embodiments the BMPER:ALK4 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 24-34 of SEQ ID NO: 26 and ends at any one of amino acids 101-126 of SEQ ID NO: 26. In some embodiments the BMPER:ALK4 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 24-34 of SEQ ID NO: 83 and ends at any one of amino acids 101-126 of SEQ ID NO: 83. In certain preferred embodiments, BMPER:ALK4 heteromultimers are soluble. In some embodiments, a BMPER:ALK4 heteromultimer of the disclosure binds to one or more TGF-beta superfamily ligands (e.g., binds to one or more TGF-beta superfamily ligands with a K_{D} of at least 1×10⁻⁷). In some embodiments, a BMPER:ALK4 heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a BMPER:ALK4 heteromultimer of the disclosure has a different TGF-beta ligand binding and/or inhibition profile (specificity) compared to a corresponding homomultimer (e.g., BMPER and ALK4 homomultimers). In some embodiments, a BMPER:ALK4 heteromultimer of the disclosure is a heterodimer.

In certain aspects, the disclosure relates to heteromultimers that comprise at least one BMPER polypeptide, which includes fragments, functional variants, and modified forms thereof, and at least one ALK5 polypeptide, which includes fragments, functional variants, and modified forms thereof. In some embodiments, the BMPER:ALK5 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 553 or 554. In some embodiments, the BMPER:ALK5 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 39-50 of SEQ ID NO: 553 and ends at any one of amino acids 364-369 of SEQ ID NO: 553. In some embodiments, the BMPER:ALK5 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 370-386 of SEQ ID NO: 553 and ends at any one of amino acids 682-685 of SEQ ID NO: 553. In some embodiments, the BMPER:ALK5 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 39-50 of SEQ ID NO: 553 and ends at any one of amino acids 682-685 of SEQ ID NO: 553. In some embodiments, the BMPER:ALK5 heteromultimer comprises a BMPER protein, wherein the BMPER protein is a dimer comprising a first polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 39-50 of SEQ ID NO: 553 and ends at any one of amino acids 364-369 of SEQ ID NO: 553, and second polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 370-386 of SEQ ID NO: 553, and ends at any one of amino acids 682-685 of SEQ ID NO: 553. In some embodiments, the BMPER:ALK5 heteromultimer comprises a single chain ligand trap that comprises a first BMPER polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 39-50 of SEQ ID NO: 553 and ends at any one of amino acids 364-369 of SEQ ID NO: 553, and second BMPER polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 370-386 of SEQ ID NO: 553 and ends at any one of amino acids 682-685 of SEQ ID NO: 553. In some embodiments, the BMPER:ALK5 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: SEQ ID NO: 30, 31, 87, or 88. In some embodiments the BMPER:ALK5 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 25-36 of SEQ ID NO: 30 and ends at any one of amino acids 101-126 of SEQ ID NO: 30. In some embodiments the BMPER:ALK5 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 25-36 of SEQ ID NO: 87 and ends at any one of amino acids 101-130 of SEQ ID NO: 87. In certain preferred embodiments, BMPER:ALK5 heteromultimers are soluble. In some embodiments, a BMPER:ALK5 heteromultimer of the disclosure binds to one or more TGF-beta superfamily ligands (e.g., binds to one or more TGF-beta superfamily ligands with a K_{D} of at least 1×10⁻⁷). In some embodiments, a BMPER:ALK5 heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a BMPER:ALK5 heteromultimer of the disclosure has a different TGF-beta ligand binding and/or inhibition profile (specificity) compared to a corresponding homomultimer (e.g., BMPER and ALK5 homomultimers). In some embodiments, a BMPER:ALK5 heteromultimer of the disclosure is a heterodimer.

In certain aspects, the disclosure relates to heteromultimers that comprise at least one BMPER polypeptide, which includes fragments, functional variants, and modified forms thereof, and at least one ALK6 polypeptide, which includes fragments, functional variants, and modified forms thereof. In some embodiments, the BMPER:ALK6 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 553 or 554. In some embodiments, the BMPER:ALK6 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 39-50 of SEQ ID NO: 553 and ends at any one of amino acids 364-369 of SEQ ID NO: 553. In some embodiments, the BMPER:ALK6 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 370-386 of SEQ ID NO: 553 and ends at any one of amino acids 682-685 of SEQ ID NO: 553. In some embodiments, the BMPER:ALK6 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 39-50 of SEQ ID NO: 553 and ends at any one of amino acids 682-685 of SEQ ID NO: 553. In some embodiments, the BMPER:ALK6 heteromultimer comprises a BMPER protein, wherein the BMPER protein is a dimer comprising a first polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 39-50 of SEQ ID NO: 553 and ends at any one of amino acids 364-369 of SEQ ID NO: 553, and second polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 370-386 of SEQ ID NO: 553, and ends at any one of amino acids 682-685 of SEQ ID NO: 553. In some embodiments, the BMPER:ALK6 heteromultimer comprises a single chain ligand trap that comprises a first BMPER polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 39-50 of SEQ ID NO: 553 and ends at any one of amino acids 364-369 of SEQ ID NO: 553, and second BMPER polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 370-386 of SEQ ID NO: 553 and ends at any one of amino acids 682-685 of SEQ ID NO: 553. In some embodiments, the BMPER:ALK6 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: SEQ ID NO: 34, 35, 91, or 92. In some embodiments the BMPER:ALK6 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 14-32 of SEQ ID NO: 34 and ends at any one of amino acids 102-126 of SEQ ID NO: 34. In some embodiments the BMPER:ALK6 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-62 of SEQ ID NO: 91 and ends at any one of amino acids 132-156 of SEQ ID NO: 91. In certain preferred embodiments, BMPER:ALK6 heteromultimers are soluble. In some embodiments, a BMPER:ALK6 heteromultimer of the disclosure binds to one or more TGF-beta superfamily ligands (e.g., binds to one or more TGF-beta superfamily ligands with a K_{D} of at least 1×10⁻⁷). In some embodiments, a BMPER:ALK6 heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a BMPER:ALK6 heteromultimer of the disclosure has a different TGF-beta ligand binding and/or inhibition profile (specificity) compared to a corresponding homomultimer (e.g., BMPER and ALK6 homomultimers). In some embodiments, a BMPER:ALK6 heteromultimer of the disclosure is a heterodimer.

In certain aspects, the disclosure relates to heteromultimers that comprise at least one BMPER polypeptide, which includes fragments, functional variants, and modified forms thereof, and at least one ALK7 polypeptide, which includes fragments, functional variants, and modified forms thereof. In some embodiments, the BMPER:ALK7 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 553 or 554. In some embodiments, the BMPER:ALK7 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 39-50 of SEQ ID NO: 553 and ends at any one of amino acids 364-369 of SEQ ID NO: 553. In some embodiments, the BMPER:ALK7 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 370-386 of SEQ ID NO: 553 and ends at any one of amino acids 682-685 of SEQ ID NO: 553. In some embodiments, the BMPER:ALK7 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 39-50 of SEQ ID NO: 553 and ends at any one of amino acids 682-685 of SEQ ID NO: 553. In some embodiments, the BMPER:ALK7 heteromultimer comprises a BMPER protein, wherein the BMPER protein is a dimer comprising a first polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 39-50 of SEQ ID NO: 553 and ends at any one of amino acids 364-369 of SEQ ID NO: 553, and second polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 370-386 of SEQ ID NO: 553, and ends at any one of amino acids 682-685 of SEQ ID NO: 553. In some embodiments, the BMPER:ALK7 heteromultimer comprises a single chain ligand trap that comprises a first BMPER polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 39-50 of SEQ ID NO: 553 and ends at any one of amino acids 364-369 of SEQ ID NO: 553, and second BMPER polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 370-386 of SEQ ID NO: 553 and ends at any one of amino acids 682-685 of SEQ ID NO: 553. In some embodiments, the BMPER:ALK7 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: SEQ ID NO: 38, 39, 301, 302, 305, 306, 309, 310, or 313. In some embodiments the BMPER:ALK7 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 21-28 of SEQ ID NO: 38 and ends at any one of amino acids 92-113 of SEQ ID NO: 38. In some embodiments the BMPER:ALK7 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-13 of SEQ ID NO: 301 and ends at any one of amino acids 42-63 of SEQ ID NO: 301. In some embodiments the BMPER:ALK7 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 21-28 of SEQ ID NO: 305 and ends at any one of amino acids 411-413 of SEQ ID NO: 305. In some embodiments the BMPER:ALK7 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 21-28 of SEQ ID NO: 309 and ends at any one of amino acids 334-336 of SEQ ID NO: 309. In certain preferred embodiments, BMPER:ALK7 heteromultimers are soluble. In some embodiments, a BMPER:ALK7 heteromultimer of the disclosure binds to one or more TGF-beta superfamily ligands (e.g., binds to one or more TGF-beta superfamily ligands with a K_{D} of at least 1×10⁻⁷). In some embodiments, a BMPER:ALK7 heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a BMPER:ALK7 heteromultimer of the disclosure has a different TGF-beta ligand binding and/or inhibition profile (specificity) compared to a corresponding homomultimer (e.g., BMPER and ALK7 homomultimers). In some embodiments, a BMPER:ALK7 heteromultimer of the disclosure is a heterodimer.

In certain aspects, the disclosure relates to heteromultimers that comprise at least one RGM-A polypeptide, which includes fragments, functional variants, and modified forms thereof, and at least one ALK1 polypeptide, which includes fragments, functional variants, and modified forms thereof. In some embodiments, the RGM-A:ALK1 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 553 or 554. In some embodiments, the RGM-A:ALK1 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-177 of SEQ ID NO: 561 and ends at any one of amino acids 430-458 of SEQ ID NO: 561. In some embodiments, the RGM-A:ALK1 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-153 of SEQ ID NO: 565 and ends at any one of amino acids 406-434 of SEQ ID NO: 565. In some embodiments, the RGM-A:ALK1 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-169 of SEQ ID NO: 569 and ends at any one of amino acids 422-450 of SEQ ID NO: 569. In some embodiments, the RGM-A:ALK1 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 14 or 15. In some embodiments, the RGM-A:ALK1 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 22-34 of SEQ ID NO: 14 and ends at any one of amino acids 95-118 of SEQ ID NO: 14. In certain preferred embodiments, RGM-A:ALK1 heteromultimers are soluble. In some embodiments, a RGM-A:ALK heteromultimer of the disclosure binds to one or more TGF-beta superfamily ligands (e.g., binds to one or more TGF-beta superfamily ligands with a K_{D} of at least 1×10⁻⁷). In some embodiments, a RGM-A:ALK1 heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a RGM-A:ALK1 heteromultimer of the disclosure has a different TGF-beta ligand binding and/or inhibition profile (specificity) compared to a corresponding homomultimer (e.g., RGM-A and ALK1 homomultimers). In some embodiments, a RGM-A:ALK1 heteromultimer of the disclosure is a heterodimer.

In certain aspects, the disclosure relates to heteromultimers that comprise at least one RGM-A polypeptide, which includes fragments, functional variants, and modified forms thereof, and at least one ALK2 polypeptide, which includes fragments, functional variants, and modified forms thereof. In some embodiments, the RGM-A:ALK2 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 553 or 554. In some embodiments, the RGM-A:ALK2 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-177 of SEQ ID NO: 561 and ends at any one of amino acids 430-458 of SEQ ID NO: 561. In some embodiments, the RGM-A:ALK2 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-153 of SEQ ID NO: 565 and ends at any one of amino acids 406-434 of SEQ ID NO: 565. In some embodiments, the RGM-A:ALK2 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-169 of SEQ ID NO: 569 and ends at any one of amino acids 422-450 of SEQ ID NO: 569. In some embodiments, the RGM-A:ALK2 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: SEQ ID NO: 18 or 19. In some embodiments the RGM-A:ALK2 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 21-35 of SEQ ID NO: 18 and ends at any one of amino acids 99-123 of SEQ ID NO: 18. In certain preferred embodiments, RGM-A:ALK2 heteromultimers are soluble. In some embodiments, a RGM-A:ALK2 heteromultimer of the disclosure binds to one or more TGF-beta superfamily ligands (e.g., binds to one or more TGF-beta superfamily ligands with a K_{D} of at least 1×10⁻⁷). In some embodiments, a RGM-A:ALK2 heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a RGM-A:ALK2 heteromultimer of the disclosure has a different TGF-beta ligand binding and/or inhibition profile (specificity) compared to a corresponding homomultimer (e.g., RGM-A and ALK2 homomultimers). In some embodiments, a RGM-A:ALK2 heteromultimer of the disclosure is a heterodimer.

In certain aspects, the disclosure relates to heteromultimers that comprise at least one RGM-A polypeptide, which includes fragments, functional variants, and modified forms thereof, and at least one ALK3 polypeptide, which includes fragments, functional variants, and modified forms thereof. In some embodiments, the RGM-A:ALK3 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 553 or 554. In some embodiments, the RGM-A:ALK3 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-177 of SEQ ID NO: 561 and ends at any one of amino acids 430-458 of SEQ ID NO: 561. In some embodiments, the RGM-A:ALK3 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-153 of SEQ ID NO: 565 and ends at any one of amino acids 406-434 of SEQ ID NO: 565. In some embodiments, the RGM-A:ALK3 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-169 of SEQ ID NO: 569 and ends at any one of amino acids 422-450 of SEQ ID NO: 569. In some embodiments, the RGM-A:ALK3 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: SEQ ID NO: 22 or 23. In some embodiments the RGM-A:ALK3 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 24-61 of SEQ ID NO: 22 and ends at any one of amino acids 130-152 of SEQ ID NO: 22. In certain preferred embodiments, RGM-A:ALK3 heteromultimers are soluble. In some embodiments, a RGM-A:ALK3 heteromultimer of the disclosure binds to one or more TGF-beta superfamily ligands (e.g., binds to one or more TGF-beta superfamily ligands with a K_{D} of at least 1×10⁻⁷). In some embodiments, a RGM-A:ALK3 heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a RGM-A:ALK3 heteromultimer of the disclosure has a different TGF-beta ligand binding and/or inhibition profile (specificity) compared to a corresponding homomultimer (e.g., RGM-A and ALK3 homomultimers). In some embodiments, a RGM-A:ALK3 heteromultimer of the disclosure is a heterodimer.

In certain aspects, the disclosure relates to heteromultimers that comprise at least one RGM-A polypeptide, which includes fragments, functional variants, and modified forms thereof, and at least one ALK4 polypeptide, which includes fragments, functional variants, and modified forms thereof. In some embodiments, the RGM-A:ALK4 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 553 or 554. In some embodiments, the RGM-A:ALK4 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-177 of SEQ ID NO: 561 and ends at any one of amino acids 430-458 of SEQ ID NO: 561. In some embodiments, the RGM-A:ALK4 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-153 of SEQ ID NO: 565 and ends at any one of amino acids 406-434 of SEQ ID NO: 565. In some embodiments, the RGM-A:ALK4 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-169 of SEQ ID NO: 569 and ends at any one of amino acids 422-450 of SEQ ID NO: 569. In some embodiments, the RGM-A:ALK4 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: SEQ ID NO: 26, 27, 83, or 84. In some embodiments the RGM-A:ALK4 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 24-34 of SEQ ID NO: 26 and ends at any one of amino acids 101-126 of SEQ ID NO: 26. In some embodiments the RGM-A:ALK4 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 24-34 of SEQ ID NO: 83 and ends at any one of amino acids 101-126 of SEQ ID NO: 83. In certain preferred embodiments, RGM-A:ALK4 heteromultimers are soluble. In some embodiments, a RGM-A:ALK4 heteromultimer of the disclosure binds to one or more TGF-beta superfamily ligands (e.g., binds to one or more TGF-beta superfamily ligands with a K_{D} of at least 1×10⁻⁷). In some embodiments, a RGM-A:ALK4 heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a RGM-A:ALK4 heteromultimer of the disclosure has a different TGF-beta ligand binding and/or inhibition profile (specificity) compared to a corresponding homomultimer (e.g., RGM-A and ALK4 homomultimers). In some embodiments, a RGM-A:ALK4 heteromultimer of the disclosure is a heterodimer.

In certain aspects, the disclosure relates to heteromultimers that comprise at least one RGM-A polypeptide, which includes fragments, functional variants, and modified forms thereof, and at least one ALK5 polypeptide, which includes fragments, functional variants, and modified forms thereof. In some embodiments, the RGM-A:ALK5 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 553 or 554. In some embodiments, the RGM-A:ALK5 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-177 of SEQ ID NO: 561 and ends at any one of amino acids 430-458 of SEQ ID NO: 561. In some embodiments, the RGM-A:ALK5 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-153 of SEQ ID NO: 565 and ends at any one of amino acids 406-434 of SEQ ID NO: 565. In some embodiments, the RGM-A:ALK5 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-169 of SEQ ID NO: 569 and ends at any one of amino acids 422-450 of SEQ ID NO: 569. In some embodiments, the RGM-A:ALK5 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: SEQ ID NO: 30, 31, 87, or 88. In some embodiments the RGM-A:ALK5 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 25-36 of SEQ ID NO: 30 and ends at any one of amino acids 101-126 of SEQ ID NO: 30. In some embodiments the RGM-A:ALK5 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 25-36 of SEQ ID NO: 87 and ends at any one of amino acids 101-130 of SEQ ID NO: 87. In certain preferred embodiments, RGM-A:ALK5 heteromultimers are soluble. In some embodiments, a RGM-A:ALK5 heteromultimer of the disclosure binds to one or more TGF-beta superfamily ligands (e.g., binds to one or more TGF-beta superfamily ligands with a K_{D} of at least 1×10⁻⁷). In some embodiments, a RGM-A:ALK5 heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a RGM-A:ALK5 heteromultimer of the disclosure has a different TGF-beta ligand binding and/or inhibition profile (specificity) compared to a corresponding homomultimer (e.g., RGM-A and ALK5 homomultimers). In some embodiments, a RGM-A:ALK5 heteromultimer of the disclosure is a heterodimer.

In certain aspects, the disclosure relates to heteromultimers that comprise at least one RGM-A polypeptide, which includes fragments, functional variants, and modified forms thereof, and at least one ALK6 polypeptide, which includes fragments, functional variants, and modified forms thereof. In some embodiments, the RGM-A:ALK6 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 553 or 554. In some embodiments, the RGM-A:ALK6 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-177 of SEQ ID NO: 561 and ends at any one of amino acids 430-458 of SEQ ID NO: 561. In some embodiments, the RGM-A:ALK6 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-153 of SEQ ID NO: 565 and ends at any one of amino acids 406-434 of SEQ ID NO: 565. In some embodiments, the RGM-A:ALK6 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-169 of SEQ ID NO: 569 and ends at any one of amino acids 422-450 of SEQ ID NO: 569. In some embodiments, the RGM-A:ALK6 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 553 or 554. In some embodiments, the RGM-A:ALK6 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-177 of SEQ ID NO: 561 and ends at any one of amino acids 430-458 of SEQ ID NO: 561. In some embodiments, the RGM-A:ALK6 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-153 of SEQ ID NO: 565 and ends at any one of amino acids 406-434 of SEQ ID NO: 565. In some embodiments, the RGM-A:ALK6 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-169 of SEQ ID NO: 569 and ends at any one of amino acids 422-450 of SEQ ID NO: 569. In certain preferred embodiments, RGM-A:ALK6 heteromultimers are soluble. In some embodiments, a RGM-A:ALK6 heteromultimer of the disclosure binds to one or more TGF-beta superfamily ligands (e.g., binds to one or more TGF-beta superfamily ligands with a K_{D} of at least 1×10⁻⁷). In some embodiments, a RGM-A:ALK6 heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a RGM-A:ALK6 heteromultimer of the disclosure has a different TGF-beta ligand binding and/or inhibition profile (specificity) compared to a corresponding homomultimer (e.g., RGM-A and ALK6 homomultimers). In some embodiments, a RGM-A:ALK6 heteromultimer of the disclosure is a heterodimer.

In certain aspects, the disclosure relates to heteromultimers that comprise at least one RGM-A polypeptide, which includes fragments, functional variants, and modified forms thereof, and at least one ALK7 polypeptide, which includes fragments, functional variants, and modified forms thereof. In some embodiments, the RGM-A:ALK7 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 553 or 554. In some embodiments, the RGM-A:ALK7 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-177 of SEQ ID NO: 561 and ends at any one of amino acids 430-458 of SEQ ID NO: 561. In some embodiments, the RGM-A:ALK7 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-153 of SEQ ID NO: 565 and ends at any one of amino acids 406-434 of SEQ ID NO: 565. In some embodiments, the RGM-A:ALK7 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-169 of SEQ ID NO: 569 and ends at any one of amino acids 422-450 of SEQ ID NO: 569. In some embodiments, the RGM-A:ALK7 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: SEQ ID NO: 38, 39, 301, 302, 305, 306, 309, 310, or 313. In some embodiments the RGM-A:ALK7 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 21-28 of SEQ ID NO: 38 and ends at any one of amino acids 92-113 of SEQ ID NO: 38. In some embodiments the RGM-A:ALK7 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-13 of SEQ ID NO: 301 and ends at any one of amino acids 42-63 of SEQ ID NO: 301. In some embodiments the RGM-A:ALK7 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 21-28 of SEQ ID NO: 305 and ends at any one of amino acids 411-413 of SEQ ID NO: 305. In some embodiments the RGM-A:ALK7 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 21-28 of SEQ ID NO: 309 and ends at any one of amino acids 334-336 of SEQ ID NO: 309. In certain preferred embodiments, RGM-A:ALK7 heteromultimers are soluble. In some embodiments, a RGM-A:ALK7 heteromultimer of the disclosure binds to one or more TGF-beta superfamily ligands (e.g., binds to one or more TGF-beta superfamily ligands with a K_{D} of at least 1×10⁻⁷). In some embodiments, a RGM-A:ALK7 heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a RGM-A:ALK7 heteromultimer of the disclosure has a different TGF-beta ligand binding and/or inhibition profile (specificity) compared to a corresponding homomultimer (e.g., RGM-A and ALK7 homomultimers). In some embodiments, a RGM-A:ALK7 heteromultimer of the disclosure is a heterodimer.

In certain aspects, the disclosure relates to heteromultimers that comprise at least one RGM-B polypeptide, which includes fragments, functional variants, and modified forms thereof, and at least one ALK1 polypeptide, which includes fragments, functional variants, and modified forms thereof. In some embodiments, the RGM-B:ALK1 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 557 or 558. In some embodiments, the RGM-B:ALK1 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-87 of SEQ ID NO: 557 and ends at any one of amino acids 452-478 of SEQ ID NO: 557. In some embodiments, the RGM-B:ALK1 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 210-222 of SEQ ID NO: 557 and ends at any one of amino acids 413-452 of SEQ ID NO: 557. In some embodiments, the RGM-B:ALK1 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 87-95 of SEQ ID NO: 557 and ends at any one of amino acids 204-209 of SEQ ID NO: 557. In some embodiments, the RGM-B:ALK1 heteromultimer comprises a RGM-B protein, wherein the RGM-B protein is a dimer comprising a first polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 87-95 of SEQ ID NO: 557 and ends at any one of amino acids 204-209 of SEQ ID NO: 557 and second polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 210-222 of SEQ ID NO: 557 and ends at any one of amino acids 413-452 of SEQ ID NO: 557. In some embodiments, the RGM-B:ALK1 heteromultimer comprises a single chain ligand trap that comprises a first RGM-B polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 87-95 of SEQ ID NO: 557 and ends at any one of amino acids 204-209 of SEQ ID NO: 557 and second RGM-B polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 210-222 of SEQ ID NO: 557 and ends at any one of amino acids 413-452 of SEQ ID NO: 557. In some embodiments, the RGM-B:ALK1 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 14 or 15. In some embodiments, the RGM-B:ALK1 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 22-34 of SEQ ID NO: 14 and ends at any one of amino acids 95-118 of SEQ ID NO: 14. In certain preferred embodiments, RGM-B:ALK1 heteromultimers are soluble. In some embodiments, a RGM-B:ALK1 heteromultimer of the disclosure binds to one or more TGF-beta superfamily ligands (e.g., binds to one or more TGF-beta superfamily ligands with a K_{D} of at least 1×10⁻⁷). In some embodiments, a RGM-B:ALK1 heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a RGM-B:ALK1 heteromultimer of the disclosure has a different TGF-beta ligand binding and/or inhibition profile (specificity) compared to a corresponding homomultimer (e.g., RGM-B and ALK1 homomultimers). In some embodiments, a RGM-B:ALK1 heteromultimer of the disclosure is a heterodimer.

In certain aspects, the disclosure relates to heteromultimers that comprise at least one RGM-B polypeptide, which includes fragments, functional variants, and modified forms thereof, and at least one ALK2 polypeptide, which includes fragments, functional variants, and modified forms thereof. In some embodiments, the RGM-B:ALK2 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 557 or 558. In some embodiments, the RGM-B:ALK2 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-87 of SEQ ID NO: 557 and ends at any one of amino acids 452-478 of SEQ ID NO: 557. In some embodiments, the RGM-B:ALK2 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 210-222 of SEQ ID NO: 557 and ends at any one of amino acids 413-452 of SEQ ID NO: 557. In some embodiments, the RGM-B:ALK2 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 87-95 of SEQ ID NO: 557 and ends at any one of amino acids 204-209 of SEQ ID NO: 557. In some embodiments, the RGM-B:ALK2 heteromultimer comprises a RGM-B protein, wherein the RGM-B protein is a dimer comprising a first polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 87-95 of SEQ ID NO: 557 and ends at any one of amino acids 204-209 of SEQ ID NO: 557 and second polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 210-222 of SEQ ID NO: 557 and ends at any one of amino acids 413-452 of SEQ ID NO: 557. In some embodiments, the RGM-B:ALK2 heteromultimer comprises a single chain ligand trap that comprises a first RGM-B polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 87-95 of SEQ ID NO: 557 and ends at any one of amino acids 204-209 of SEQ ID NO: 557 and second RGM-B polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 210-222 of SEQ ID NO: 557 and ends at any one of amino acids 413-452 of SEQ ID NO: 557. In some embodiments, the RGM-B:ALK2 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: SEQ ID NO: 18 or 19. In some embodiments the RGM-B:ALK2 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 21-35 of SEQ ID NO: 18 and ends at any one of amino acids 99-123 of SEQ ID NO: 18. In certain preferred embodiments, RGM-B:ALK2 heteromultimers are soluble. In some embodiments, a RGM-B:ALK2 heteromultimer of the disclosure binds to one or more TGF-beta superfamily ligands (e.g., binds to one or more TGF-beta superfamily ligands with a K_{D} of at least 1×10⁻⁷). In some embodiments, a RGM-B:ALK2 heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a RGM-B:ALK2 heteromultimer of the disclosure has a different TGF-beta ligand binding and/or inhibition profile (specificity) compared to a corresponding homomultimer (e.g., RGM-B and ALK2 homomultimers). In some embodiments, a RGM-B:ALK2 heteromultimer of the disclosure is a heterodimer.

In certain aspects, the disclosure relates to heteromultimers that comprise at least one RGM-B polypeptide, which includes fragments, functional variants, and modified forms thereof, and at least one ALK3 polypeptide, which includes fragments, functional variants, and modified forms thereof. In some embodiments, the RGM-B:ALK3 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 557 or 558. In some embodiments, the RGM-B:ALK3 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-87 of SEQ ID NO: 557 and ends at any one of amino acids 452-478 of SEQ ID NO: 557. In some embodiments, the RGM-B:ALK3 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 210-222 of SEQ ID NO: 557 and ends at any one of amino acids 413-452 of SEQ ID NO: 557. In some embodiments, the RGM-B:ALK3 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 87-95 of SEQ ID NO: 557 and ends at any one of amino acids 204-209 of SEQ ID NO: 557. In some embodiments, the RGM-B:ALK3 heteromultimer comprises a RGM-B protein, wherein the RGM-B protein is a dimer comprising a first polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 87-95 of SEQ ID NO: 557 and ends at any one of amino acids 204-209 of SEQ ID NO: 557 and second polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 210-222 of SEQ ID NO: 557 and ends at any one of amino acids 413-452 of SEQ ID NO: 557. In some embodiments, the RGM-B:ALK3 heteromultimer comprises a single chain ligand trap that comprises a first RGM-B polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 87-95 of SEQ ID NO: 557 and ends at any one of amino acids 204-209 of SEQ ID NO: 557 and second RGM-B polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 210-222 of SEQ ID NO: 557 and ends at any one of amino acids 413-452 of SEQ ID NO: 557. In some embodiments, the RGM-B:ALK3 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: SEQ ID NO: 22 or 23. In some embodiments the RGM-B:ALK3 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 24-61 of SEQ ID NO: 22 and ends at any one of amino acids 130-152 of SEQ ID NO: 22. In certain preferred embodiments, RGM-B:ALK3 heteromultimers are soluble. In some embodiments, a RGM-B:ALK3 heteromultimer of the disclosure binds to one or more TGF-beta superfamily ligands (e.g., binds to one or more TGF-beta superfamily ligands with a K_{D} of at least 1×10⁻⁷). In some embodiments, a RGM-B:ALK3 heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a RGM-B:ALK3 heteromultimer of the disclosure has a different TGF-beta ligand binding and/or inhibition profile (specificity) compared to a corresponding homomultimer (e.g., RGM-B and ALK3 homomultimers). In some embodiments, a RGM-B:ALK3 heteromultimer of the disclosure is a heterodimer.

In certain aspects, the disclosure relates to heteromultimers that comprise at least one RGM-B polypeptide, which includes fragments, functional variants, and modified forms thereof, and at least one ALK4 polypeptide, which includes fragments, functional variants, and modified forms thereof. In some embodiments, the RGM-B:ALK4 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 557 or 558. In some embodiments, the RGM-B:ALK4 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-87 of SEQ ID NO: 557 and ends at any one of amino acids 452-478 of SEQ ID NO: 557. In some embodiments, the RGM-B:ALK4 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 210-222 of SEQ ID NO: 557 and ends at any one of amino acids 413-452 of SEQ ID NO: 557. In some embodiments, the RGM-B:ALK4 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 87-95 of SEQ ID NO: 557 and ends at any one of amino acids 204-209 of SEQ ID NO: 557. In some embodiments, the RGM-B:ALK4 heteromultimer comprises a RGM-B protein, wherein the RGM-B protein is a dimer comprising a first polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 87-95 of SEQ ID NO: 557 and ends at any one of amino acids 204-209 of SEQ ID NO: 557 and second polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 210-222 of SEQ ID NO: 557 and ends at any one of amino acids 413-452 of SEQ ID NO: 557. In some embodiments, the RGM-B:ALK4 heteromultimer comprises a single chain ligand trap that comprises a first RGM-B polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 87-95 of SEQ ID NO: 557 and ends at any one of amino acids 204-209 of SEQ ID NO: 557 and second RGM-B polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 210-222 of SEQ ID NO: 557 and ends at any one of amino acids 413-452 of SEQ ID NO: 557. In some embodiments, the RGM-B:ALK4 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: SEQ ID NO: 26, 27, 83, or 84. In some embodiments the RGM-B:ALK4 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 24-34 of SEQ ID NO: 26 and ends at any one of amino acids 101-126 of SEQ ID NO: 26. In some embodiments the RGM-B:ALK4 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 24-34 of SEQ ID NO: 83 and ends at any one of amino acids 101-126 of SEQ ID NO: 83. In certain preferred embodiments, RGM-B:ALK4 heteromultimers are soluble. In some embodiments, a RGM-B:ALK4 heteromultimer of the disclosure binds to one or more TGF-beta superfamily ligands (e.g., binds to one or more TGF-beta superfamily ligands with a K_{D} of at least 1×10⁻⁷). In some embodiments, a RGM-B:ALK4 heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a RGM-B:ALK4 heteromultimer of the disclosure has a different TGF-beta ligand binding and/or inhibition profile (specificity) compared to a corresponding homomultimer (e.g., RGM-B and ALK4 homomultimers). In some embodiments, a RGM-B:ALK4 heteromultimer of the disclosure is a heterodimer.

In certain aspects, the disclosure relates to heteromultimers that comprise at least one RGM-B polypeptide, which includes fragments, functional variants, and modified forms thereof, and at least one ALK5 polypeptide, which includes fragments, functional variants, and modified forms thereof. In some embodiments, the RGM-B:ALK5 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 557 or 558. In some embodiments, the RGM-B:ALK5 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-87 of SEQ ID NO: 557 and ends at any one of amino acids 452-478 of SEQ ID NO: 557. In some embodiments, the RGM-B:ALK5 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 210-222 of SEQ ID NO: 557 and ends at any one of amino acids 413-452 of SEQ ID NO: 557. In some embodiments, the RGM-B:ALK5 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 87-95 of SEQ ID NO: 557 and ends at any one of amino acids 204-209 of SEQ ID NO: 557. In some embodiments, the RGM-B:ALK5 heteromultimer comprises a RGM-B protein, wherein the RGM-B protein is a dimer comprising a first polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 87-95 of SEQ ID NO: 557 and ends at any one of amino acids 204-209 of SEQ ID NO: 557 and second polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 210-222 of SEQ ID NO: 557 and ends at any one of amino acids 413-452 of SEQ ID NO: 557. In some embodiments, the RGM-B:ALK5 heteromultimer comprises a single chain ligand trap that comprises a first RGM-B polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 87-95 of SEQ ID NO: 557 and ends at any one of amino acids 204-209 of SEQ ID NO: 557 and second RGM-B polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 210-222 of SEQ ID NO: 557 and ends at any one of amino acids 413-452 of SEQ ID NO: 557. In some embodiments, the RGM-B:ALK5 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: SEQ ID NO: 30, 31, 87, or 88. In some embodiments the RGM-B:ALK5 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 25-36 of SEQ ID NO: 30 and ends at any one of amino acids 101-126 of SEQ ID NO: 30. In some embodiments the RGM-B:ALK5 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 25-36 of SEQ ID NO: 87 and ends at any one of amino acids 101-130 of SEQ ID NO: 87. In certain preferred embodiments, RGM-B:ALK5 heteromultimers are soluble. In some embodiments, a RGM-B:ALK5 heteromultimer of the disclosure binds to one or more TGF-beta superfamily ligands (e.g., binds to one or more TGF-beta superfamily ligands with a K_{D} of at least 1×10⁻⁷). In some embodiments, a RGM-B:ALK5 heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a RGM-B:ALK5 heteromultimer of the disclosure has a different TGF-beta ligand binding and/or inhibition profile (specificity) compared to a corresponding homomultimer (e.g., RGM-B and ALK5 homomultimers). In some embodiments, a RGM-B:ALK5 heteromultimer of the disclosure is a heterodimer.

In certain aspects, the disclosure relates to heteromultimers that comprise at least one RGM-B polypeptide, which includes fragments, functional variants, and modified forms thereof, and at least one ALK6 polypeptide, which includes fragments, functional variants, and modified forms thereof. In some embodiments, the RGM-B:ALK6 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 557 or 558. In some embodiments, the RGM-B:ALK6 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-87 of SEQ ID NO: 557 and ends at any one of amino acids 452-478 of SEQ ID NO: 557. In some embodiments, the RGM-B:ALK6 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 210-222 of SEQ ID NO: 557 and ends at any one of amino acids 413-452 of SEQ ID NO: 557. In some embodiments, the RGM-B:ALK6 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 87-95 of SEQ ID NO: 557 and ends at any one of amino acids 204-209 of SEQ ID NO: 557. In some embodiments, the RGM-B:ALK6 heteromultimer comprises a RGM-B protein, wherein the RGM-B protein is a dimer comprising a first polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 87-95 of SEQ ID NO: 557 and ends at any one of amino acids 204-209 of SEQ ID NO: 557 and second polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 210-222 of SEQ ID NO: 557 and ends at any one of amino acids 413-452 of SEQ ID NO: 557. In some embodiments, the RGM-B:ALK6 heteromultimer comprises a single chain ligand trap that comprises a first RGM-B polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 87-95 of SEQ ID NO: 557 and ends at any one of amino acids 204-209 of SEQ ID NO: 557 and second RGM-B polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 210-222 of SEQ ID NO: 557 and ends at any one of amino acids 413-452 of SEQ ID NO: 557. In some embodiments, the RGM-B:ALK6 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: SEQ ID NO: 34, 35, 91, or 92. In some embodiments the RGM-B:ALK6 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 14-32 of SEQ ID NO: 34 and ends at any one of amino acids 102-126 of SEQ ID NO: 34. In some embodiments the RGM-B:ALK6 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-62 of SEQ ID NO: 91 and ends at any one of amino acids 132-156 of SEQ ID NO: 91. In certain preferred embodiments, RGM-B:ALK6 heteromultimers are soluble. In some embodiments, a RGM-B:ALK6 heteromultimer of the disclosure binds to one or more TGF-beta superfamily ligands (e.g., binds to one or more TGF-beta superfamily ligands with a K_{D} of at least 1×10⁻⁷). In some embodiments, a RGM-B:ALK6 heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a RGM-B:ALK6 heteromultimer of the disclosure has a different TGF-beta ligand binding and/or inhibition profile (specificity) compared to a corresponding homomultimer (e.g., RGM-B and ALK6 homomultimers). In some embodiments, a RGM-B:ALK6 heteromultimer of the disclosure is a heterodimer.

In certain aspects, the disclosure relates to heteromultimers that comprise at least one RGM-B polypeptide, which includes fragments, functional variants, and modified forms thereof, and at least one ALK7 polypeptide, which includes fragments, functional variants, and modified forms thereof. In some embodiments, the RGM-B:ALK7 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 557 or 558. In some embodiments, the RGM-B:ALK7 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-87 of SEQ ID NO: 557 and ends at any one of amino acids 452-478 of SEQ ID NO: 557. In some embodiments, the RGM-B:ALK7 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 210-222 of SEQ ID NO: 557 and ends at any one of amino acids 413-452 of SEQ ID NO: 557. In some embodiments, the RGM-B:ALK7 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 87-95 of SEQ ID NO: 557 and ends at any one of amino acids 204-209 of SEQ ID NO: 557. In some embodiments, the RGM-B:ALK7 heteromultimer comprises a RGM-B protein, wherein the RGM-B protein is a dimer comprising a first polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 87-95 of SEQ ID NO: 557 and ends at any one of amino acids 204-209 of SEQ ID NO: 557 and second polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 210-222 of SEQ ID NO: 557 and ends at any one of amino acids 413-452 of SEQ ID NO: 557. In some embodiments, the RGM-B:ALK7 heteromultimer comprises a single chain ligand trap that comprises a first RGM-B polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 87-95 of SEQ ID NO: 557 and ends at any one of amino acids 204-209 of SEQ ID NO: 557 and second RGM-B polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 210-222 of SEQ ID NO: 557 and ends at any one of amino acids 413-452 of SEQ ID NO: 557. In some embodiments, the RGM-B:ALK7 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: SEQ ID NO: 38, 39, 301, 302, 305, 306, 309, 310, or 313. In some embodiments the RGM-B:ALK7 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 21-28 of SEQ ID NO: 38 and ends at any one of amino acids 92-113 of SEQ ID NO: 38. In some embodiments the RGM-B:ALK7 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-13 of SEQ ID NO: 301 and ends at any one of amino acids 42-63 of SEQ ID NO: 301. In some embodiments the RGM-B:ALK7 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 21-28 of SEQ ID NO: 305 and ends at any one of amino acids 411-413 of SEQ ID NO: 305. In some embodiments the RGM-B:ALK7 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 21-28 of SEQ ID NO: 309 and ends at any one of amino acids 334-336 of SEQ ID NO: 309. In certain preferred embodiments, RGM-B:ALK7 heteromultimers are soluble. In some embodiments, a RGM-B:ALK7 heteromultimer of the disclosure binds to one or more TGF-beta superfamily ligands (e.g., binds to one or more TGF-beta superfamily ligands with a K_{D} of at least 1×10⁻⁷). In some embodiments, a RGM-B:ALK7 heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a RGM-B:ALK7 heteromultimer of the disclosure has a different TGF-beta ligand binding and/or inhibition profile (specificity) compared to a corresponding homomultimer (e.g., RGM-B and ALK7 homomultimers). In some embodiments, a RGM-B:ALK7 heteromultimer of the disclosure is a heterodimer.

In certain aspects, the disclosure relates to heteromultimers that comprise at least one hemojuvelin polypeptide, which includes fragments, functional variants, and modified forms thereof, and at least one ALK1 polypeptide, which includes fragments, functional variants, and modified forms thereof. In some embodiments, the hemojuvelin:ALK1 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 573, 574, 577, 578, 581, or 582. In some embodiments, the hemojuvelin:ALK1 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-36 of SEQ ID NO: 573 and ends at any one of amino acids 400-426 of SEQ ID NO: 573. In some embodiments, the hemojuvelin:ALK1 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 36-42 of SEQ ID NO: 573 and ends at any one of amino acids 167-172 of SEQ ID NO: 573. In some embodiments, the hemojuvelin:ALK1 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 173-185 of SEQ ID NO: 573 and ends at any one of amino acids 361-400 of SEQ ID NO: 573. In some embodiments, the hemojuvelin:ALK1 heteromultimer comprises a hemojuvelin protein that is a dimer comprising a first polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 36-42 of SEQ ID NO: 573, and ends at any one of amino acids 167-172 of SEQ ID NO: 573 and second polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 173-185 of SEQ ID NO: 573 and ends at any one of amino acids 361-400 of SEQ ID NO: 573. In some embodiments, the hemojuvelin:ALK1 heteromultimer comprises a single chain ligand trap that comprises a first hemojuvelin polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 36-42 of SEQ ID NO: 573 and ends at any one of amino acids 167-172 of SEQ ID NO: 573 and second hemojuvelin polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 173-185 of SEQ ID NO: 573 and ends at any one of amino acids 361-400 of SEQ ID NO: 573. In some embodiments, the hemojuvelin:ALK1 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-6 of SEQ ID NO: 577 and ends at any one of amino acids 287-313 of SEQ ID NO: 577. In some embodiments, the hemojuvelin:ALK1 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-6 of SEQ ID NO: 577 and ends at any one of amino acids 54-59 of SEQ ID NO: 577. In some embodiments, the hemojuvelin:ALK1 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 60-72 of SEQ ID NO: 577 and ends at any one of amino acids 248-287 of SEQ ID NO: 577. In some embodiments, the hemojuvelin:ALK1 heteromultimer comprises a hemojuvelin protein, wherein the hemojuvelin protein is a dimer comprising a first polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 1-6 of SEQ ID NO: 577 and ends at any one of amino acids 54-59 of SEQ ID NO: 577, and second polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 60-72 of SEQ ID NO: 577 and ends at any one of amino acids 248-287 of SEQ ID NO: 577. In some embodiments, the hemojuvelin:ALK1 heteromultimer comprises a single chain ligand trap that comprises a first hemojuvelin polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 1-6 of SEQ ID NO: 577 and ends at any one of amino acids 54-59 of SEQ ID NO: 577, and second hemojuvelin polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 60-72 of SEQ ID NO: 577, and ends at any one of amino acids 248-287 of SEQ ID NO: 577. In some embodiments, the hemojuvelin:ALK1 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-4 of SEQ ID NO: 581 and ends at any one of amino acids 135-200 of SEQ ID NO: 581. In some embodiments, the hemojuvelin:ALK1 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 14 or 15. In some embodiments, the hemojuvelin:ALK1 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 22-34 of SEQ ID NO: 14 and ends at any one of amino acids 95-118 of SEQ ID NO: 14. In certain preferred embodiments, hemojuvelin:ALK1 heteromultimers are soluble. In some embodiments, a hemojuvelin:ALK heteromultimer of the disclosure binds to one or more TGF-beta superfamily ligands (e.g., binds to one or more TGF-beta superfamily ligands with a K_{D} of at least 1×10⁻⁷). In some embodiments, a hemojuvelin:ALK1 heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a hemojuvelin:ALK1 heteromultimer of the disclosure has a different TGF-beta ligand binding and/or inhibition profile (specificity) compared to a corresponding homomultimer (e.g., hemojuvelin and ALK1 homomultimers). In some embodiments, a hemojuvelin:ALK1 heteromultimer of the disclosure is a heterodimer.

In certain aspects, the disclosure relates to heteromultimers that comprise at least one hemojuvelin polypeptide, which includes fragments, functional variants, and modified forms thereof, and at least one ALK2 polypeptide, which includes fragments, functional variants, and modified forms thereof. In some embodiments, the hemojuvelin:ALK2 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 573, 574, 577, 578, 581, or 582. In some embodiments, the hemojuvelin:ALK2 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-36 of SEQ ID NO: 573 and ends at any one of amino acids 400-426 of SEQ ID NO: 573. In some embodiments, the hemojuvelin:ALK2 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 36-42 of SEQ ID NO: 573 and ends at any one of amino acids 167-172 of SEQ ID NO: 573. In some embodiments, the hemojuvelin:ALK2 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 173-185 of SEQ ID NO: 573 and ends at any one of amino acids 361-400 of SEQ ID NO: 573. In some embodiments, the hemojuvelin:ALK2 heteromultimer comprises a hemojuvelin protein that is a dimer comprising a first polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 36-42 of SEQ ID NO: 573, and ends at any one of amino acids 167-172 of SEQ ID NO: 573 and second polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 173-185 of SEQ ID NO: 573 and ends at any one of amino acids 361-400 of SEQ ID NO: 573. In some embodiments, the hemojuvelin:ALK2 heteromultimer comprises a single chain ligand trap that comprises a first hemojuvelin polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 36-42 of SEQ ID NO: 573 and ends at any one of amino acids 167-172 of SEQ ID NO: 573 and second hemojuvelin polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 173-185 of SEQ ID NO: 573 and ends at any one of amino acids 361-400 of SEQ ID NO: 573. In some embodiments, the hemojuvelin:ALK2 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-6 of SEQ ID NO: 577 and ends at any one of amino acids 287-313 of SEQ ID NO: 577. In some embodiments, the hemojuvelin:ALK2 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-6 of SEQ ID NO: 577 and ends at any one of amino acids 54-59 of SEQ ID NO: 577. In some embodiments, the hemojuvelin:ALK2 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 60-72 of SEQ ID NO: 577 and ends at any one of amino acids 248-287 of SEQ ID NO: 577. In some embodiments, the hemojuvelin:ALK2 heteromultimer comprises a hemojuvelin protein, wherein the hemojuvelin protein is a dimer comprising a first polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 1-6 of SEQ ID NO: 577 and ends at any one of amino acids 54-59 of SEQ ID NO: 577, and second polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 60-72 of SEQ ID NO: 577 and ends at any one of amino acids 248-287 of SEQ ID NO: 577. In some embodiments, the hemojuvelin:ALK2 heteromultimer comprises a single chain ligand trap that comprises a first hemojuvelin polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 1-6 of SEQ ID NO: 577 and ends at any one of amino acids 54-59 of SEQ ID NO: 577, and second hemojuvelin polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 60-72 of SEQ ID NO: 577, and ends at any one of amino acids 248-287 of SEQ ID NO: 577. In some embodiments, the hemojuvelin:ALK2 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-4 of SEQ ID NO: 581 and ends at any one of amino acids 135-200 of SEQ ID NO: 581. In some embodiments, the hemojuvelin:ALK2 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: SEQ ID NO: 18 or 19. In some embodiments the hemojuvelin:ALK2 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 21-35 of SEQ ID NO: 18 and ends at any one of amino acids 99-123 of SEQ ID NO: 18. In certain preferred embodiments, hemojuvelin:ALK2 heteromultimers are soluble. In some embodiments, a hemojuvelin:ALK2 heteromultimer of the disclosure binds to one or more TGF-beta superfamily ligands (e.g., binds to one or more TGF-beta superfamily ligands with a K_{D} of at least 1×10⁻⁷). In some embodiments, a hemojuvelin:ALK2 heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a hemojuvelin:ALK2 heteromultimer of the disclosure has a different TGF-beta ligand binding and/or inhibition profile (specificity) compared to a corresponding homomultimer (e.g., hemojuvelin and ALK2 homomultimers). In some embodiments, a hemojuvelin:ALK2 heteromultimer of the disclosure is a heterodimer.

In certain aspects, the disclosure relates to heteromultimers that comprise at least one hemojuvelin polypeptide, which includes fragments, functional variants, and modified forms thereof, and at least one ALK3 polypeptide, which includes fragments, functional variants, and modified forms thereof. In some embodiments, the hemojuvelin:ALK3 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 573, 574, 577, 578, 581, or 582. In some embodiments, the hemojuvelin:ALK3 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-36 of SEQ ID NO: 573 and ends at any one of amino acids 400-426 of SEQ ID NO: 573. In some embodiments, the hemojuvelin:ALK3 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 36-42 of SEQ ID NO: 573 and ends at any one of amino acids 167-172 of SEQ ID NO: 573. In some embodiments, the hemojuvelin:ALK3 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 173-185 of SEQ ID NO: 573 and ends at any one of amino acids 361-400 of SEQ ID NO: 573. In some embodiments, the hemojuvelin:ALK3 heteromultimer comprises a hemojuvelin protein that is a dimer comprising a first polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 36-42 of SEQ ID NO: 573, and ends at any one of amino acids 167-172 of SEQ ID NO: 573 and second polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 173-185 of SEQ ID NO: 573 and ends at any one of amino acids 361-400 of SEQ ID NO: 573. In some embodiments, the hemojuvelin:ALK3 heteromultimer comprises a single chain ligand trap that comprises a first hemojuvelin polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 36-42 of SEQ ID NO: 573 and ends at any one of amino acids 167-172 of SEQ ID NO: 573 and second hemojuvelin polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 173-185 of SEQ ID NO: 573 and ends at any one of amino acids 361-400 of SEQ ID NO: 573. In some embodiments, the hemojuvelin:ALK3 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-6 of SEQ ID NO: 577 and ends at any one of amino acids 287-313 of SEQ ID NO: 577. In some embodiments, the hemojuvelin:ALK3 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-6 of SEQ ID NO: 577 and ends at any one of amino acids 54-59 of SEQ ID NO: 577. In some embodiments, the hemojuvelin:ALK3 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 60-72 of SEQ ID NO: 577 and ends at any one of amino acids 248-287 of SEQ ID NO: 577. In some embodiments, the hemojuvelin:ALK3 heteromultimer comprises a hemojuvelin protein, wherein the hemojuvelin protein is a dimer comprising a first polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 1-6 of SEQ ID NO: 577 and ends at any one of amino acids 54-59 of SEQ ID NO: 577, and second polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 60-72 of SEQ ID NO: 577 and ends at any one of amino acids 248-287 of SEQ ID NO: 577. In some embodiments, the hemojuvelin:ALK3 heteromultimer comprises a single chain ligand trap that comprises a first hemojuvelin polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 1-6 of SEQ ID NO: 577 and ends at any one of amino acids 54-59 of SEQ ID NO: 577, and second hemojuvelin polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 60-72 of SEQ ID NO: 577, and ends at any one of amino acids 248-287 of SEQ ID NO: 577. In some embodiments, the hemojuvelin:ALK3 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-4 of SEQ ID NO: 581 and ends at any one of amino acids 135-200 of SEQ ID NO: 581. In some embodiments, the hemojuvelin:ALK3 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: SEQ ID NO: 22 or 23. In some embodiments the hemojuvelin:ALK3 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 24-61 of SEQ ID NO: 22 and ends at any one of amino acids 130-152 of SEQ ID NO: 22. In certain preferred embodiments, hemojuvelin:ALK3 heteromultimers are soluble. In some embodiments, a hemojuvelin:ALK3 heteromultimer of the disclosure binds to one or more TGF-beta superfamily ligands (e.g., binds to one or more TGF-beta superfamily ligands with a K_{D} of at least 1×10⁻⁷). In some embodiments, a hemojuvelin:ALK3 heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a hemojuvelin:ALK3 heteromultimer of the disclosure has a different TGF-beta ligand binding and/or inhibition profile (specificity) compared to a corresponding homomultimer (e.g., hemojuvelin and ALK3 homomultimers). In some embodiments, a hemojuvelin:ALK3 heteromultimer of the disclosure is a heterodimer.

In certain aspects, the disclosure relates to heteromultimers that comprise at least one hemojuvelin polypeptide, which includes fragments, functional variants, and modified forms thereof, and at least one ALK4 polypeptide, which includes fragments, functional variants, and modified forms thereof. In some embodiments, the hemojuvelin:ALK4 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 573, 574, 577, 578, 581, or 582. In some embodiments, the hemojuvelin:ALK4 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-36 of SEQ ID NO: 573 and ends at any one of amino acids 400-426 of SEQ ID NO: 573. In some embodiments, the hemojuvelin:ALK4 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 36-42 of SEQ ID NO: 573 and ends at any one of amino acids 167-172 of SEQ ID NO: 573. In some embodiments, the hemojuvelin:ALK4 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 173-185 of SEQ ID NO: 573 and ends at any one of amino acids 361-400 of SEQ ID NO: 573. In some embodiments, the hemojuvelin:ALK4 heteromultimer comprises a hemojuvelin protein that is a dimer comprising a first polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 36-42 of SEQ ID NO: 573, and ends at any one of amino acids 167-172 of SEQ ID NO: 573 and second polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 173-185 of SEQ ID NO: 573 and ends at any one of amino acids 361-400 of SEQ ID NO: 573. In some embodiments, the hemojuvelin:ALK4 heteromultimer comprises a single chain ligand trap that comprises a first hemojuvelin polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 36-42 of SEQ ID NO: 573 and ends at any one of amino acids 167-172 of SEQ ID NO: 573 and second hemojuvelin polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 173-185 of SEQ ID NO: 573 and ends at any one of amino acids 361-400 of SEQ ID NO: 573. In some embodiments, the hemojuvelin:ALK4 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-6 of SEQ ID NO: 577 and ends at any one of amino acids 287-313 of SEQ ID NO: 577. In some embodiments, the hemojuvelin:ALK4 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-6 of SEQ ID NO: 577 and ends at any one of amino acids 54-59 of SEQ ID NO: 577. In some embodiments, the hemojuvelin:ALK4 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 60-72 of SEQ ID NO: 577 and ends at any one of amino acids 248-287 of SEQ ID NO: 577. In some embodiments, the hemojuvelin:ALK4 heteromultimer comprises a hemojuvelin protein, wherein the hemojuvelin protein is a dimer comprising a first polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 1-6 of SEQ ID NO: 577 and ends at any one of amino acids 54-59 of SEQ ID NO: 577, and second polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 60-72 of SEQ ID NO: 577 and ends at any one of amino acids 248-287 of SEQ ID NO: 577. In some embodiments, the hemojuvelin:ALK4 heteromultimer comprises a single chain ligand trap that comprises a first hemojuvelin polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 1-6 of SEQ ID NO: 577 and ends at any one of amino acids 54-59 of SEQ ID NO: 577, and second hemojuvelin polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 60-72 of SEQ ID NO: 577, and ends at any one of amino acids 248-287 of SEQ ID NO: 577. In some embodiments, the hemojuvelin:ALK4 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-4 of SEQ ID NO: 581 and ends at any one of amino acids 135-200 of SEQ ID NO: 581. In some embodiments, the hemojuvelin:ALK4 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: SEQ ID NO: 26, 27, 83, or 84. In some embodiments the hemojuvelin:ALK4 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 24-34 of SEQ ID NO: 26 and ends at any one of amino acids 101-126 of SEQ ID NO: 26. In some embodiments the hemojuvelin:ALK4 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 24-34 of SEQ ID NO: 83 and ends at any one of amino acids 101-126 of SEQ ID NO: 83. In certain preferred embodiments, hemojuvelin:ALK4 heteromultimers are soluble. In some embodiments, a hemojuvelin:ALK4 heteromultimer of the disclosure binds to one or more TGF-beta superfamily ligands (e.g., binds to one or more TGF-beta superfamily ligands with a K_{D} of at least 1×10⁻⁷). In some embodiments, a hemojuvelin:ALK4 heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a hemojuvelin:ALK4 heteromultimer of the disclosure has a different TGF-beta ligand binding and/or inhibition profile (specificity) compared to a corresponding homomultimer (e.g., hemojuvelin and ALK4 homomultimers). In some embodiments, a hemojuvelin:ALK4 heteromultimer of the disclosure is a heterodimer.

In certain aspects, the disclosure relates to heteromultimers that comprise at least one hemojuvelin polypeptide, which includes fragments, functional variants, and modified forms thereof, and at least one ALK5 polypeptide, which includes fragments, functional variants, and modified forms thereof. In some embodiments, the hemojuvelin:ALK5 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 573, 574, 577, 578, 581, or 582. In some embodiments, the hemojuvelin:ALK5 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-36 of SEQ ID NO: 573 and ends at any one of amino acids 400-426 of SEQ ID NO: 573. In some embodiments, the hemojuvelin:ALK5 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 36-42 of SEQ ID NO: 573 and ends at any one of amino acids 167-172 of SEQ ID NO: 573. In some embodiments, the hemojuvelin:ALK5 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 173-185 of SEQ ID NO: 573 and ends at any one of amino acids 361-400 of SEQ ID NO: 573. In some embodiments, the hemojuvelin:ALK5 heteromultimer comprises a hemojuvelin protein that is a dimer comprising a first polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 36-42 of SEQ ID NO: 573, and ends at any one of amino acids 167-172 of SEQ ID NO: 573 and second polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 173-185 of SEQ ID NO: 573 and ends at any one of amino acids 361-400 of SEQ ID NO: 573. In some embodiments, the hemojuvelin:ALK5 heteromultimer comprises a single chain ligand trap that comprises a first hemojuvelin polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 36-42 of SEQ ID NO: 573 and ends at any one of amino acids 167-172 of SEQ ID NO: 573 and second hemojuvelin polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 173-185 of SEQ ID NO: 573 and ends at any one of amino acids 361-400 of SEQ ID NO: 573. In some embodiments, the hemojuvelin:ALK5 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-6 of SEQ ID NO: 577 and ends at any one of amino acids 287-313 of SEQ ID NO: 577. In some embodiments, the hemojuvelin:ALK5 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-6 of SEQ ID NO: 577 and ends at any one of amino acids 54-59 of SEQ ID NO: 577. In some embodiments, the hemojuvelin:ALK5 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 60-72 of SEQ ID NO: 577 and ends at any one of amino acids 248-287 of SEQ ID NO: 577. In some embodiments, the hemojuvelin:ALK5 heteromultimer comprises a hemojuvelin protein, wherein the hemojuvelin protein is a dimer comprising a first polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 1-6 of SEQ ID NO: 577 and ends at any one of amino acids 54-59 of SEQ ID NO: 577, and second polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 60-72 of SEQ ID NO: 577 and ends at any one of amino acids 248-287 of SEQ ID NO: 577. In some embodiments, the hemojuvelin:ALK5 heteromultimer comprises a single chain ligand trap that comprises a first hemojuvelin polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 1-6 of SEQ ID NO: 577 and ends at any one of amino acids 54-59 of SEQ ID NO: 577, and second hemojuvelin polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 60-72 of SEQ ID NO: 577, and ends at any one of amino acids 248-287 of SEQ ID NO: 577. In some embodiments, the hemojuvelin:ALK5 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-4 of SEQ ID NO: 581 and ends at any one of amino acids 135-200 of SEQ ID NO: 581. In some embodiments, the hemojuvelin:ALK5 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: SEQ ID NO: 30, 31, 87, or 88. In some embodiments the hemojuvelin:ALK5 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 25-36 of SEQ ID NO: 30 and ends at any one of amino acids 101-126 of SEQ ID NO: 30. In some embodiments the hemojuvelin:ALK5 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 25-36 of SEQ ID NO: 87 and ends at any one of amino acids 101-130 of SEQ ID NO: 87. In certain preferred embodiments, hemojuvelin:ALK5 heteromultimers are soluble. In some embodiments, a hemojuvelin:ALK5 heteromultimer of the disclosure binds to one or more TGF-beta superfamily ligands (e.g., binds to one or more TGF-beta superfamily ligands with a K_{D} of at least 1×10⁻⁷). In some embodiments, a hemojuvelin:ALK5 heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a hemojuvelin:ALK5 heteromultimer of the disclosure has a different TGF-beta ligand binding and/or inhibition profile (specificity) compared to a corresponding homomultimer (e.g., hemojuvelin and ALK5 homomultimers). In some embodiments, a hemojuvelin:ALK5 heteromultimer of the disclosure is a heterodimer.

In certain aspects, the disclosure relates to heteromultimers that comprise at least one hemojuvelin polypeptide, which includes fragments, functional variants, and modified forms thereof, and at least one ALK6 polypeptide, which includes fragments, functional variants, and modified forms thereof. In some embodiments, the hemojuvelin:ALK6 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 573, 574, 577, 578, 581, or 582. In some embodiments, the hemojuvelin:ALK6 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-36 of SEQ ID NO: 573 and ends at any one of amino acids 400-426 of SEQ ID NO: 573. In some embodiments, the hemojuvelin:ALK6 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 36-42 of SEQ ID NO: 573 and ends at any one of amino acids 167-172 of SEQ ID NO: 573. In some embodiments, the hemojuvelin:ALK6 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 173-185 of SEQ ID NO: 573 and ends at any one of amino acids 361-400 of SEQ ID NO: 573. In some embodiments, the hemojuvelin:ALK6 heteromultimer comprises a hemojuvelin protein that is a dimer comprising a first polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 36-42 of SEQ ID NO: 573, and ends at any one of amino acids 167-172 of SEQ ID NO: 573 and second polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 173-185 of SEQ ID NO: 573 and ends at any one of amino acids 361-400 of SEQ ID NO: 573. In some embodiments, the hemojuvelin:ALK6 heteromultimer comprises a single chain ligand trap that comprises a first hemojuvelin polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 36-42 of SEQ ID NO: 573 and ends at any one of amino acids 167-172 of SEQ ID NO: 573 and second hemojuvelin polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 173-185 of SEQ ID NO: 573 and ends at any one of amino acids 361-400 of SEQ ID NO: 573. In some embodiments, the hemojuvelin:ALK6 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-6 of SEQ ID NO: 577 and ends at any one of amino acids 287-313 of SEQ ID NO: 577. In some embodiments, the hemojuvelin:ALK6 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-6 of SEQ ID NO: 577 and ends at any one of amino acids 54-59 of SEQ ID NO: 577. In some embodiments, the hemojuvelin:ALK6 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 60-72 of SEQ ID NO: 577 and ends at any one of amino acids 248-287 of SEQ ID NO: 577. In some embodiments, the hemojuvelin:ALK6 heteromultimer comprises a hemojuvelin protein, wherein the hemojuvelin protein is a dimer comprising a first polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 1-6 of SEQ ID NO: 577 and ends at any one of amino acids 54-59 of SEQ ID NO: 577, and second polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 60-72 of SEQ ID NO: 577 and ends at any one of amino acids 248-287 of SEQ ID NO: 577. In some embodiments, the hemojuvelin:ALK6 heteromultimer comprises a single chain ligand trap that comprises a first hemojuvelin polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 1-6 of SEQ ID NO: 577 and ends at any one of amino acids 54-59 of SEQ ID NO: 577, and second hemojuvelin polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 60-72 of SEQ ID NO: 577, and ends at any one of amino acids 248-287 of SEQ ID NO: 577. In some embodiments, the hemojuvelin:ALK6 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-4 of SEQ ID NO: 581 and ends at any one of amino acids 135-200 of SEQ ID NO: 581. In some embodiments, the hemojuvelin:ALK6 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: SEQ ID NO: 34, 35, 91, or 92. In some embodiments the hemojuvelin:ALK6 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 14-32 of SEQ ID NO: 34 and ends at any one of amino acids 102-126 of SEQ ID NO: 34. In some embodiments the hemojuvelin:ALK6 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-62 of SEQ ID NO: 91 and ends at any one of amino acids 132-156 of SEQ ID NO: 91. In certain preferred embodiments, hemojuvelin:ALK6 heteromultimers are soluble. In some embodiments, a hemojuvelin:ALK6 heteromultimer of the disclosure binds to one or more TGF-beta superfamily ligands (e.g., binds to one or more TGF-beta superfamily ligands with a K_{D} of at least 1×10⁻⁷). In some embodiments, a hemojuvelin:ALK6 heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a hemojuvelin:ALK6 heteromultimer of the disclosure has a different TGF-beta ligand binding and/or inhibition profile (specificity) compared to a corresponding homomultimer (e.g., hemojuvelin and ALK6 homomultimers). In some embodiments, a hemojuvelin:ALK6 heteromultimer of the disclosure is a heterodimer.

In certain aspects, the disclosure relates to heteromultimers that comprise at least one hemojuvelin polypeptide, which includes fragments, functional variants, and modified forms thereof, and at least one ALK7 polypeptide, which includes fragments, functional variants, and modified forms thereof. In some embodiments, the hemojuvelin:ALK7 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 573, 574, 577, 578, 581, or 582. In some embodiments, the hemojuvelin:ALK7 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-36 of SEQ ID NO: 573 and ends at any one of amino acids 400-426 of SEQ ID NO: 573. In some embodiments, the hemojuvelin:ALK7 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 36-42 of SEQ ID NO: 573 and ends at any one of amino acids 167-172 of SEQ ID NO: 573. In some embodiments, the hemojuvelin:ALK7 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 173-185 of SEQ ID NO: 573 and ends at any one of amino acids 361-400 of SEQ ID NO: 573. In some embodiments, the hemojuvelin:ALK7 heteromultimer comprises a hemojuvelin protein that is a dimer comprising a first polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 36-42 of SEQ ID NO: 573, and ends at any one of amino acids 167-172 of SEQ ID NO: 573 and second polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 173-185 of SEQ ID NO: 573 and ends at any one of amino acids 361-400 of SEQ ID NO: 573. In some embodiments, the hemojuvelin:ALK7 heteromultimer comprises a single chain ligand trap that comprises a first hemojuvelin polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 36-42 of SEQ ID NO: 573 and ends at any one of amino acids 167-172 of SEQ ID NO: 573 and second hemojuvelin polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 173-185 of SEQ ID NO: 573 and ends at any one of amino acids 361-400 of SEQ ID NO: 573. In some embodiments, the hemojuvelin:ALK7 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-6 of SEQ ID NO: 577 and ends at any one of amino acids 287-313 of SEQ ID NO: 577. In some embodiments, the hemojuvelin:ALK7 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-6 of SEQ ID NO: 577 and ends at any one of amino acids 54-59 of SEQ ID NO: 577. In some embodiments, the hemojuvelin:ALK7 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 60-72 of SEQ ID NO: 577 and ends at any one of amino acids 248-287 of SEQ ID NO: 577. In some embodiments, the hemojuvelin:ALK7 heteromultimer comprises a hemojuvelin protein, wherein the hemojuvelin protein is a dimer comprising a first polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 1-6 of SEQ ID NO: 577 and ends at any one of amino acids 54-59 of SEQ ID NO: 577, and second polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 60-72 of SEQ ID NO: 577 and ends at any one of amino acids 248-287 of SEQ ID NO: 577. In some embodiments, the hemojuvelin:ALK7 heteromultimer comprises a single chain ligand trap that comprises a first hemojuvelin polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 1-6 of SEQ ID NO: 577 and ends at any one of amino acids 54-59 of SEQ ID NO: 577, and second hemojuvelin polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 60-72 of SEQ ID NO: 577, and ends at any one of amino acids 248-287 of SEQ ID NO: 577. In some embodiments, the hemojuvelin:ALK7 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-4 of SEQ ID NO: 581 and ends at any one of amino acids 135-200 of SEQ ID NO: 581. In some embodiments, the hemojuvelin:ALK7 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: SEQ ID NO: 38, 39, 301, 302, 305, 306, 309, 310, or 313. In some embodiments the hemojuvelin:ALK7 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 21-28 of SEQ ID NO: 38 and ends at any one of amino acids 92-113 of SEQ ID NO: 38. In some embodiments the hemojuvelin:ALK7 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-13 of SEQ ID NO: 301 and ends at any one of amino acids 42-63 of SEQ ID NO: 301. In some embodiments the hemojuvelin:ALK7 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 21-28 of SEQ ID NO: 305 and ends at any one of amino acids 411-413 of SEQ ID NO: 305. In some embodiments the hemojuvelin:ALK7 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 21-28 of SEQ ID NO: 309 and ends at any one of amino acids 334-336 of SEQ ID NO: 309. In certain preferred embodiments, hemojuvelin:ALK7 heteromultimers are soluble. In some embodiments, a hemojuvelin:ALK7 heteromultimer of the disclosure binds to one or more TGF-beta superfamily ligands (e.g., binds to one or more TGF-beta superfamily ligands with a K_{D} of at least 1×10⁻⁷). In some embodiments, a hemojuvelin:ALK7 heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a hemojuvelin:ALK7 heteromultimer of the disclosure has a different TGF-beta ligand binding and/or inhibition profile (specificity) compared to a corresponding homomultimer (e.g., hemojuvelin and ALK7 homomultimers). In some embodiments, a hemojuvelin:ALK7 heteromultimer of the disclosure is a heterodimer.

In certain aspects, the disclosure relates to heteromultimers that comprise at least one endoglin polypeptide, which includes fragments, functional variants, and modified forms thereof, and at least one ActRIIA polypeptide, which includes fragments, functional variants, and modified forms thereof. In some embodiments, the endoglin:ActRIIA heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 501, 502, 505, 506, 509, 510, 593, or 594. In some embodiments, the endoglin:ActRIIA heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-30 of SEQ ID NO: 501 and ends at any one of amino acids 330-346 of SEQ ID NO: 501. In some embodiments, the endoglin:ActRIIA heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-30 of SEQ ID NO: 505 and ends at any one of amino acids 330-346 of SEQ ID NO: 505. In some embodiments, the endoglin:ActRIIA heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-25 of SEQ ID NO: 509, and ends at any one of amino acids 148-164 of SEQ ID NO: 509. In some embodiments, a endoglin:ActRIIA heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 9, 10, and 11. In some embodiments, a endoglin:ActRIIA heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 21-30 of SEQ ID NO: 9, and ends at any one of amino acids 110-135 of SEQ ID NO: 9. In certain preferred embodiments, endoglin:ActRIIA heteromultimers are soluble. In some embodiments, a endoglin:ActRIIA heteromultimer of the disclosure binds to one or more TGF-beta superfamily ligands (e.g., binds to one or more TGF-beta superfamily ligands with a K_{D} of at least 1×10⁻⁷). In some embodiments, a endoglin:ActRIIA heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a endoglin:ActRIIA heteromultimer of the disclosure has a different TGF-beta ligand binding and/or inhibition profile (specificity) compared to a corresponding homomultimer (e.g., endoglin and ActRIIA homomultimers). In some embodiments, a endoglin:ActRIIA heteromultimer of the disclosure is a heterodimer.

In certain aspects, the disclosure relates to heteromultimers that comprise at least one endoglin polypeptide, which includes fragments, functional variants, and modified forms thereof, and at least one ActRIIB polypeptide, which includes fragments, functional variants, and modified forms thereof. In some embodiments, the endoglin:ActRIIB heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 501, 502, 505, 506, 509, 510, 593, or 594. In some embodiments, the endoglin:ActRIIB heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-30 of SEQ ID NO: 501 and ends at any one of amino acids 330-346 of SEQ ID NO: 501. In some embodiments, the endoglin:ActRIIB heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-30 of SEQ ID NO: 505 and ends at any one of amino acids 330-346 of SEQ ID NO: 505. In some embodiments, the endoglin:ActRIIB heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-25 of SEQ ID NO: 509, and ends at any one of amino acids 148-164 of SEQ ID NO: 509. In some embodiments, a endoglin:ActRIIB heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 1, 2, 3, 4, 5, and 6. In some embodiments, a endoglin:ActRIIB heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids 20-29 of SEQ ID NO: 1 and ends at a position corresponding to any one of amino acids 109-134 of SEQ ID NO: 1. In some embodiments, a endoglin:ActRIIB heteromultimer comprises an ActRIIB polypeptide wherein the amino acid position corresponding to L79 of SEQ ID NO: 1 is not an acidic amino acid. In certain preferred embodiments, endoglin:ActRIIB heteromultimers are soluble. In some embodiments, a endoglin:ActRIIB heteromultimer of the disclosure binds to one or more TGF-beta superfamily ligands (e.g., binds to one or more TGF-beta superfamily ligands with a K_{D} of at least 1×10⁻⁷). In some embodiments, a endoglin:ActRIIB heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a endoglin:ActRIIB heteromultimer of the disclosure has a different TGF-beta ligand binding and/or inhibition profile (specificity) compared to a corresponding homomultimer (e.g., endoglin and ActRIIB homomultimers). In some embodiments, a endoglin:ActRIIB heteromultimer of the disclosure is a heterodimer.

In certain aspects, the disclosure relates to heteromultimers that comprise at least one endoglin polypeptide, which includes fragments, functional variants, and modified forms thereof, and at least one TGFBRII polypeptide, which includes fragments, functional variants, and modified forms thereof. In some embodiments, the endoglin:TGFBRII heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 501, 502, 505, 506, 509, 510, 593, or 594. In some embodiments, the endoglin:TGFBRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-30 of SEQ ID NO: 501 and ends at any one of amino acids 330-346 of SEQ ID NO: 501. In some embodiments, the endoglin:TGFBRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-30 of SEQ ID NO: 505 and ends at any one of amino acids 330-346 of SEQ ID NO: 505. In some embodiments, the endoglin:TGFBRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-25 of SEQ ID NO: 509, and ends at any one of amino acids 148-164 of SEQ ID NO: 509. In some embodiments, a endoglin:TGFBRII heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 42, 43, 67, or 68. In some embodiments, a endoglin:TGFBRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 23-51 of SEQ ID NO: 42 and ends at any one of amino acids 143-166 of SEQ ID NO: 42. In some embodiments, a endoglin:TGFBRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 23-44 of SEQ ID NO: 67 and ends at any one of amino acids 168-191 of SEQ ID NO: 67. In certain preferred embodiments, endoglin:TGFBRII heteromultimers are soluble. In some embodiments, a endoglin:TGFBRII heteromultimer of the disclosure binds to one or more TGF-beta superfamily ligands (e.g., binds to one or more TGF-beta superfamily ligands with a K_{D} of at least 1×10⁻⁷). In some embodiments, a endoglin:TGFBRII heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a endoglin:TGFBRII heteromultimer of the disclosure has a different TGF-beta ligand binding and/or inhibition profile (specificity) compared to a corresponding homomultimer (e.g., endoglin and TGFBRII homomultimers). In some embodiments, a endoglin:TGFBRII heteromultimer of the disclosure is a heterodimer.

In certain aspects, the disclosure relates to heteromultimers that comprise at least one endoglin polypeptide, which includes fragments, functional variants, and modified forms thereof, and at least one BMPRII polypeptide, which includes fragments, functional variants, and modified forms thereof. In some embodiments, the endoglin:BMPRII heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 501, 502, 505, 506, 509, 510, 593, or 594. In some embodiments, the endoglin:BMPRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-30 of SEQ ID NO: 501 and ends at any one of amino acids 330-346 of SEQ ID NO: 501. In some embodiments, the endoglin:BMPRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-30 of SEQ ID NO: 505 and ends at any one of amino acids 330-346 of SEQ ID NO: 505. In some embodiments, the endoglin:BMPRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-25 of SEQ ID NO: 509, and ends at any one of amino acids 148-164 of SEQ ID NO: 509. In some embodiments, a endoglin:BMPRII heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 46, 47, 71, or 72. In some embodiments, a endoglin:BMPRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 27-34 of SEQ ID NO: 46 and ends at any one of amino acids 123-150 of SEQ ID NO: 46. In some embodiments, a endoglin:BMPRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 27-34 of SEQ ID NO: 71 and ends at any one of amino acids 123-150 of SEQ ID NO: 71. In certain preferred embodiments, endoglin:BMPRII heteromultimers are soluble. In some embodiments, a endoglin:BMPRII heteromultimer of the disclosure binds to one or more TGF-beta superfamily ligands (e.g., binds to one or more TGF-beta superfamily ligands with a K_{D} of at least 1×10⁻⁷). In some embodiments, a endoglin:BMPRII heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a endoglin:BMPRII heteromultimer of the disclosure has a different TGF-beta ligand binding and/or inhibition profile (specificity) compared to a corresponding homomultimer (e.g., endoglin and BMPRII homomultimers). In some embodiments, a endoglin:BMPRII heteromultimer of the disclosure is a heterodimer.

In certain aspects, the disclosure relates to heteromultimers that comprise at least one endoglin polypeptide, which includes fragments, functional variants, and modified forms thereof, and at least one MISRII polypeptide, which includes fragments, functional variants, and modified forms thereof. In some embodiments, the endoglin:MISRII heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 501, 502, 505, 506, 509, 510, 593, or 594. In some embodiments, the endoglin:MISRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-30 of SEQ ID NO: 501 and ends at any one of amino acids 330-346 of SEQ ID NO: 501. In some embodiments, the endoglin:MISRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-30 of SEQ ID NO: 505 and ends at any one of amino acids 330-346 of SEQ ID NO: 505. In some embodiments, the endoglin:MISRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-25 of SEQ ID NO: 509, and ends at any one of amino acids 148-164 of SEQ ID NO: 509. In some embodiments, a endoglin:MISRII heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 50, 51, 75, 76, 79, or 80. In some embodiments, a endoglin:MISRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 17-24 of SEQ ID NO: 50 and ends at any one of amino acids 116-149 of SEQ ID NO: 50. In some embodiments, a endoglin:MISRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 17-24 of SEQ ID NO: 75 and ends at any one of amino acids 116-149 of SEQ ID NO: 75. In some embodiments, a endoglin:MISRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 17-24 of SEQ ID NO: 50 and ends at any one of amino acids 116-149 of SEQ ID NO: 79. In certain preferred embodiments, endoglin:MISRII heteromultimers are soluble. In some embodiments, a endoglin:MISRII heteromultimer of the disclosure binds to one or more TGF-beta superfamily ligands (e.g., binds to one or more TGF-beta superfamily ligands with a K_{D} of at least 1×10⁻⁷). In some embodiments, a endoglin:MISRII heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a endoglin:MISRII heteromultimer of the disclosure has a different TGF-beta ligand binding and/or inhibition profile (specificity) compared to a corresponding homomultimer (e.g., endoglin and MISRII homomultimers). In some embodiments, a endoglin:MISRII heteromultimer of the disclosure is a heterodimer.

In certain aspects, the disclosure relates to heteromultimers that comprise at least one betaglycan polypeptide, which includes fragments, functional variants, and modified forms thereof, and at least one ActRIIA polypeptide, which includes fragments, functional variants, and modified forms thereof. In some embodiments, the betaglycan:ActRIIA heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 585, 586, 589, or 590. In some embodiments, the betaglycan:ActRIIA heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 21-28 of SEQ ID NO: 585 and ends at any one of amino acids 381-787 of SEQ ID NO: 585. In some embodiments, the betaglycan:ActRIIA heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 21-28 of SEQ ID NO: 589 and ends at any one of amino acids 380-786 of SEQ ID NO: 589. In some embodiments, a betaglycan:ActRIIA heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 9, 10, and 11. In some embodiments, a betaglycan:ActRIIA heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 21-30 of SEQ ID NO: 9, and ends at any one of amino acids 110-135 of SEQ ID NO: 9. In certain preferred embodiments, betaglycan:ActRIIA heteromultimers are soluble. In some embodiments, a betaglycan:ActRIIA heteromultimer of the disclosure binds to one or more TGF-beta superfamily ligands (e.g., binds to one or more TGF-beta superfamily ligands with a K_{D} of at least 1×10⁻⁷). In some embodiments, a betaglycan:ActRIIA heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a betaglycan:ActRIIA heteromultimer of the disclosure has a different TGF-beta ligand binding and/or inhibition profile (specificity) compared to a corresponding homomultimer (e.g., betaglycan and ActRIIA homomultimers). In some embodiments, a betaglycan:ActRIIA heteromultimer of the disclosure is a heterodimer.

In certain aspects, the disclosure relates to heteromultimers that comprise at least one betaglycan polypeptide, which includes fragments, functional variants, and modified forms thereof, and at least one ActRIIB polypeptide, which includes fragments, functional variants, and modified forms thereof. In some embodiments, the betaglycan:ActRIIB heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 585, 586, 589, or 590. In some embodiments, the betaglycan:ActRIIB heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 21-28 of SEQ ID NO: 585 and ends at any one of amino acids 381-787 of SEQ ID NO: 585. In some embodiments, the betaglycan:ActRIIB heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 21-28 of SEQ ID NO: 589 and ends at any one of amino acids 380-786 of SEQ ID NO: 589. In some embodiments, a betaglycan:ActRIIB heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 1, 2, 3, 4, 5, and 6. In some embodiments, a betaglycan:ActRIIB heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids 20-29 of SEQ ID NO: 1 and ends at a position corresponding to any one of amino acids 109-134 of SEQ ID NO: 1. In some embodiments, a betaglycan:ActRIIB heteromultimer comprises an ActRIIB polypeptide wherein the amino acid position corresponding to L79 of SEQ ID NO: 1 is not an acidic amino acid. In certain preferred embodiments, betaglycan:ActRIIB heteromultimers are soluble. In some embodiments, a betaglycan:ActRIIB heteromultimer of the disclosure binds to one or more TGF-beta superfamily ligands (e.g., binds to one or more TGF-beta superfamily ligands with a K_{D} of at least 1×10⁻⁷). In some embodiments, a betaglycan:ActRIIB heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a betaglycan:ActRIIB heteromultimer of the disclosure has a different TGF-beta ligand binding and/or inhibition profile (specificity) compared to a corresponding homomultimer (e.g., betaglycan and ActRIIB homomultimers). In some embodiments, a betaglycan:ActRIIB heteromultimer of the disclosure is a heterodimer.

In certain aspects, the disclosure relates to heteromultimers that comprise at least one betaglycan polypeptide, which includes fragments, functional variants, and modified forms thereof, and at least one TGFBRII polypeptide, which includes fragments, functional variants, and modified forms thereof. In some embodiments, the betaglycan:TGFBRII heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 585, 586, 589, or 590. In some embodiments, the betaglycan:TGFBRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 21-28 of SEQ ID NO: 585 and ends at any one of amino acids 381-787 of SEQ ID NO: 585. In some embodiments, the betaglycan:TGFBRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 21-28 of SEQ ID NO: 589 and ends at any one of amino acids 380-786 of SEQ ID NO: 589. In some embodiments, a betaglycan:TGFBRII heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 42, 43, 67, or 68. In some embodiments, a betaglycan:TGFBRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 23-51 of SEQ ID NO: 42 and ends at any one of amino acids 143-166 of SEQ ID NO: 42. In some embodiments, a betaglycan:TGFBRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 23-44 of SEQ ID NO: 67 and ends at any one of amino acids 168-191 of SEQ ID NO: 67. In certain preferred embodiments, betaglycan:TGFBRII heteromultimers are soluble. In some embodiments, a betaglycan:TGFBRII heteromultimer of the disclosure binds to one or more TGF-beta superfamily ligands (e.g., binds to one or more TGF-beta superfamily ligands with a K_{D} of at least 1×10⁻⁷). In some embodiments, a betaglycan:TGFBRII heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a betaglycan:TGFBRII heteromultimer of the disclosure has a different TGF-beta ligand binding and/or inhibition profile (specificity) compared to a corresponding homomultimer (e.g., betaglycan and TGFBRII homomultimers). In some embodiments, a betaglycan:TGFBRII heteromultimer of the disclosure is a heterodimer.

In certain aspects, the disclosure relates to heteromultimers that comprise at least one betaglycan polypeptide, which includes fragments, functional variants, and modified forms thereof, and at least one BMPRII polypeptide, which includes fragments, functional variants, and modified forms thereof. In some embodiments, the betaglycan:BMPRII heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 585, 586, 589, or 590. In some embodiments, the betaglycan:BMPRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 21-28 of SEQ ID NO: 585 and ends at any one of amino acids 381-787 of SEQ ID NO: 585. In some embodiments, the betaglycan:BMPRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 21-28 of SEQ ID NO: 589 and ends at any one of amino acids 380-786 of SEQ ID NO: 589. In some embodiments, a betaglycan:BMPRII heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 46, 47, 71, or 72. In some embodiments, a betaglycan:BMPRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 27-34 of SEQ ID NO: 46 and ends at any one of amino acids 123-150 of SEQ ID NO: 46. In some embodiments, a betaglycan:BMPRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 27-34 of SEQ ID NO: 71 and ends at any one of amino acids 123-150 of SEQ ID NO: 71. In certain preferred embodiments, betaglycan:BMPRII heteromultimers are soluble. In some embodiments, a betaglycan:BMPRII heteromultimer of the disclosure binds to one or more TGF-beta superfamily ligands (e.g., binds to one or more TGF-beta superfamily ligands with a K_{D} of at least 1×10⁻⁷). In some embodiments, a betaglycan:BMPRII heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a betaglycan:BMPRII heteromultimer of the disclosure has a different TGF-beta ligand binding and/or inhibition profile (specificity) compared to a corresponding homomultimer (e.g., betaglycan and BMPRII homomultimers). In some embodiments, a betaglycan:BMPRII heteromultimer of the disclosure is a heterodimer.

In certain aspects, the disclosure relates to heteromultimers that comprise at least one betaglycan polypeptide, which includes fragments, functional variants, and modified forms thereof, and at least one MISRII polypeptide, which includes fragments, functional variants, and modified forms thereof. In some embodiments, the betaglycan:MISRII heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 585, 586, 589, or 590. In some embodiments, the betaglycan:MISRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 21-28 of SEQ ID NO: 585 and ends at any one of amino acids 381-787 of SEQ ID NO: 585. In some embodiments, the betaglycan:MISRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 21-28 of SEQ ID NO: 589 and ends at any one of amino acids 380-786 of SEQ ID NO: 589. In some embodiments, a betaglycan:MISRII heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 50, 51, 75, 76, 79, or 80. In some embodiments, a betaglycan:MISRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 17-24 of SEQ ID NO: 50 and ends at any one of amino acids 116-149 of SEQ ID NO: 50. In some embodiments, a betaglycan:MISRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 17-24 of SEQ ID NO: 75 and ends at any one of amino acids 116-149 of SEQ ID NO: 75. In some embodiments, a betaglycan:MISRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 17-24 of SEQ ID NO: 50 and ends at any one of amino acids 116-149 of SEQ ID NO: 79. In certain preferred embodiments, betaglycan:MISRII heteromultimers are soluble. In some embodiments, a betaglycan:MISRII heteromultimer of the disclosure binds to one or more TGF-beta superfamily ligands (e.g., binds to one or more TGF-beta superfamily ligands with a K_{D} of at least 1×10⁻⁷). In some embodiments, a betaglycan:MISRII heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a betaglycan:MISRII heteromultimer of the disclosure has a different TGF-beta ligand binding and/or inhibition profile (specificity) compared to a corresponding homomultimer (e.g., betaglycan and MISRII homomultimers). In some embodiments, a betaglycan:MISRII heteromultimer of the disclosure is a heterodimer.

In certain aspects, the disclosure relates to heteromultimers that comprise at least one Cripto-1 polypeptide, which includes fragments, functional variants, and modified forms thereof, and at least one ActRIIA polypeptide, which includes fragments, functional variants, and modified forms thereof. In some embodiments, the Cripto-1:ActRIIA heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 513, 514, 517, or 518. In some embodiments, the Cripto-1:ActRIIA heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 31-82 of SEQ ID NO: 513 and ends at any one of amino acids 172-188 of SEQ ID NO: 513. In some embodiments, the Cripto-1:ActRIIA heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 15-66 of SEQ ID NO: 517, and ends at any one of amino acids 156-172 of SEQ ID NO: 517. In some embodiments, a Cripto-1:ActRIIA heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 9, 10, and 11. In some embodiments, a Cripto-1:ActRIIA heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 21-30 of SEQ ID NO: 9, and ends at any one of amino acids 110-135 of SEQ ID NO: 9. In certain preferred embodiments, Cripto-1:ActRIIA heteromultimers are soluble. In some embodiments, a Cripto-1:ActRIIA heteromultimer of the disclosure binds to one or more TGF-beta superfamily ligands (e.g., binds to one or more TGF-beta superfamily ligands with a K_{D} of at least 1×10⁻⁷). In some embodiments, a Cripto-1:ActRIIA heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a Cripto-1:ActRIIA heteromultimer of the disclosure has a different TGF-beta ligand binding and/or inhibition profile (specificity) compared to a corresponding homomultimer (e.g., Cripto-1 and ActRIIA homomultimers). In some embodiments, a Cripto-1:ActRIIA heteromultimer of the disclosure is a heterodimer.

In certain aspects, the disclosure relates to heteromultimers that comprise at least one Cripto-1 polypeptide, which includes fragments, functional variants, and modified forms thereof, and at least one ActRIIB polypeptide, which includes fragments, functional variants, and modified forms thereof. In some embodiments, the Cripto-1:ActRIIB heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 513, 514, 517, or 518. In some embodiments, the Cripto-1:ActRIIB heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 31-82 of SEQ ID NO: 513 and ends at any one of amino acids 172-188 of SEQ ID NO: 513. In some embodiments, the Cripto-1:ActRIIB heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 15-66 of SEQ ID NO: 517, and ends at any one of amino acids 156-172 of SEQ ID NO: 517. In some embodiments, a Cripto-1:ActRIIB heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 1, 2, 3, 4, 5, and 6. In some embodiments, a Cripto-1:ActRIIB heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids 20-29 of SEQ ID NO: 1 and ends at a position corresponding to any one of amino acids 109-134 of SEQ ID NO: 1. In some embodiments, a Cripto-1:ActRIIB heteromultimer comprises an ActRIIB polypeptide wherein the amino acid position corresponding to L79 of SEQ ID NO: 1 is not an acidic amino acid. In some embodiments, a Cripto-1:ActRIIB heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 1, 2, 3, 4, 5, and 6. In some embodiments, a Cripto-1:ActRIIB heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids 20-29 of SEQ ID NO: 1 and ends at a position corresponding to any one of amino acids 109-134 of SEQ ID NO: 1. In some embodiments, a Cripto-1:ActRIIB heteromultimer comprises an ActRIIB polypeptide wherein the amino acid position corresponding to L79 of SEQ ID NO: 1 is not an acidic amino acid. In certain preferred embodiments, Cripto-1:ActRIIB heteromultimers are soluble. In some embodiments, a Cripto-1:ActRIIB heteromultimer of the disclosure binds to one or more TGF-beta superfamily ligands (e.g., binds to one or more TGF-beta superfamily ligands with a K_{D} of at least 1×10⁻⁷). In some embodiments, a Cripto-1:ActRIIB heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a Cripto-1:ActRIIB heteromultimer of the disclosure has a different TGF-beta ligand binding and/or inhibition profile (specificity) compared to a corresponding homomultimer (e.g., Cripto-1 and ActRIIB homomultimers). In some embodiments, a Cripto-1:ActRIIB heteromultimer of the disclosure is a heterodimer.

In certain aspects, the disclosure relates to heteromultimers that comprise at least one Cripto-1 polypeptide, which includes fragments, functional variants, and modified forms thereof, and at least one TGFBRII polypeptide, which includes fragments, functional variants, and modified forms thereof. In some embodiments, the Cripto-1:TGFBRII heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 513, 514, 517, or 518. In some embodiments, the Cripto-1:TGFBRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 31-82 of SEQ ID NO: 513 and ends at any one of amino acids 172-188 of SEQ ID NO: 513. In some embodiments, the Cripto-1:TGFBRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 15-66 of SEQ ID NO: 517, and ends at any one of amino acids 156-172 of SEQ ID NO: 517. In some embodiments, a Cripto-1:TGFBRII heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 42, 43, 67, or 68. In some embodiments, a Cripto-1:TGFBRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 23-51 of SEQ ID NO: 42 and ends at any one of amino acids 143-166 of SEQ ID NO: 42. In some embodiments, a Cripto-1:TGFBRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 23-44 of SEQ ID NO: 67 and ends at any one of amino acids 168-191 of SEQ ID NO: 67. In certain preferred embodiments, Cripto-1:TGFBRII heteromultimers are soluble. In some embodiments, a Cripto-1:TGFBRII heteromultimer of the disclosure binds to one or more TGF-beta superfamily ligands (e.g., binds to one or more TGF-beta superfamily ligands with a K_{D} of at least 1×10⁻⁷). In some embodiments, a Cripto-1:TGFBRII heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a Cripto-1:TGFBRII heteromultimer of the disclosure has a different TGF-beta ligand binding and/or inhibition profile (specificity) compared to a corresponding homomultimer (e.g., Cripto-1 and TGFBRII homomultimers). In some embodiments, a Cripto-1:TGFBRII heteromultimer of the disclosure is a heterodimer.

In certain aspects, the disclosure relates to heteromultimers that comprise at least one Cripto-1 polypeptide, which includes fragments, functional variants, and modified forms thereof, and at least one BMPRII polypeptide, which includes fragments, functional variants, and modified forms thereof. In some embodiments, the Cripto-1:BMPRII heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 513, 514, 517, or 518. In some embodiments, the Cripto-1:BMPRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 31-82 of SEQ ID NO: 513 and ends at any one of amino acids 172-188 of SEQ ID NO: 513. In some embodiments, the Cripto-1:BMPRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 15-66 of SEQ ID NO: 517, and ends at any one of amino acids 156-172 of SEQ ID NO: 517. In some embodiments, a Cripto-1:BMPRII heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 46, 47, 71, or 72. In some embodiments, a Cripto-1:BMPRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 27-34 of SEQ ID NO: 46 and ends at any one of amino acids 123-150 of SEQ ID NO: 46. In some embodiments, a Cripto-1:BMPRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 27-34 of SEQ ID NO: 71 and ends at any one of amino acids 123-150 of SEQ ID NO: 71. In certain preferred embodiments, Cripto-1:BMPRII heteromultimers are soluble. In some embodiments, a Cripto-1:BMPRII heteromultimer of the disclosure binds to one or more TGF-beta superfamily ligands (e.g., binds to one or more TGF-beta superfamily ligands with a K_{D} of at least 1×10⁻⁷). In some embodiments, a Cripto-1:BMPRII heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a Cripto-1:BMPRII heteromultimer of the disclosure has a different TGF-beta ligand binding and/or inhibition profile (specificity) compared to a corresponding homomultimer (e.g., Cripto-1 and BMPRII homomultimers). In some embodiments, a Cripto-1:BMPRII heteromultimer of the disclosure is a heterodimer.

In certain aspects, the disclosure relates to heteromultimers that comprise at least one Cripto-1 polypeptide, which includes fragments, functional variants, and modified forms thereof, and at least one MISRII polypeptide, which includes fragments, functional variants, and modified forms thereof. In some embodiments, the Cripto-1:MISRII heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 513, 514, 517, or 518. In some embodiments, the Cripto-1:MISRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 31-82 of SEQ ID NO: 513 and ends at any one of amino acids 172-188 of SEQ ID NO: 513. In some embodiments, the Cripto-1:MISRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 15-66 of SEQ ID NO: 517, and ends at any one of amino acids 156-172 of SEQ ID NO: 517. In some embodiments, a Cripto-1:MISRII heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 50, 51, 75, 76, 79, or 80. In some embodiments, a Cripto-1:MISRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 17-24 of SEQ ID NO: 50 and ends at any one of amino acids 116-149 of SEQ ID NO: 50. In some embodiments, a Cripto-1:MISRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 17-24 of SEQ ID NO: 75 and ends at any one of amino acids 116-149 of SEQ ID NO: 75. In some embodiments, a Cripto-1:MISRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 17-24 of SEQ ID NO: 50 and ends at any one of amino acids 116-149 of SEQ ID NO: 79. In certain preferred embodiments, Cripto-1:MISRII heteromultimers are soluble. In some embodiments, a Cripto-1:MISRII heteromultimer of the disclosure binds to one or more TGF-beta superfamily ligands (e.g., binds to one or more TGF-beta superfamily ligands with a K_{D} of at least 1×10⁻⁷). In some embodiments, a Cripto-1:MISRII heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a Cripto-1:MISRII heteromultimer of the disclosure has a different TGF-beta ligand binding and/or inhibition profile (specificity) compared to a corresponding homomultimer (e.g., Cripto-1 and MISRII homomultimers). In some embodiments, a Cripto-1:MISRII heteromultimer of the disclosure is a heterodimer.

In certain aspects, the disclosure relates to heteromultimers that comprise at least one Cryptic protein polypeptide, which includes fragments, functional variants, and modified forms thereof, and at least one ActRIIA polypeptide, which includes fragments, functional variants, and modified forms thereof. In some embodiments, the Cryptic protein:ActRIIA heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 521, 522, 525, 526, 529, or 530. In some embodiments, the Cryptic protein:ActRIIA heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-90 of SEQ ID NO: 521 and ends at any one of amino acids 157-233 of SEQ ID NO: 521. In some embodiments, the Cryptic protein:ActRIIA heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-30 of SEQ ID NO: 525 and ends at any one of amino acids 82-191 of SEQ ID NO: 525. In some embodiments, the Cryptic protein:ActRIIA heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-30 of SEQ ID NO: 529, and ends at any one of amino acids 82-148 of SEQ ID NO: 529. In some embodiments, a Cryptic protein:ActRIIA heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 9, 10, and 11. In some embodiments, a Cryptic protein:ActRIIA heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 21-30 of SEQ ID NO: 9, and ends at any one of amino acids 110-135 of SEQ ID NO: 9. In certain preferred embodiments, Cryptic protein:ActRIIA heteromultimers are soluble. In some embodiments, a Cryptic protein:ActRIIA heteromultimer of the disclosure binds to one or more TGF-beta superfamily ligands (e.g., binds to one or more TGF-beta superfamily ligands with a K_{D} of at least 1×10⁻⁷). In some embodiments, a Cryptic protein:ActRIIA heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a Cryptic protein:ActRIIA heteromultimer of the disclosure has a different TGF-beta ligand binding and/or inhibition profile (specificity) compared to a corresponding homomultimer (e.g., Cryptic protein and ActRIIA homomultimers). In some embodiments, a Cryptic protein:ActRIIA heteromultimer of the disclosure is a heterodimer.

In certain aspects, the disclosure relates to heteromultimers that comprise at least one Cryptic protein polypeptide, which includes fragments, functional variants, and modified forms thereof, and at least one ActRIIB polypeptide, which includes fragments, functional variants, and modified forms thereof. In some embodiments, the Cryptic protein:ActRIIB heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 521, 522, 525, 526, 529, or 530. In some embodiments, the Cryptic protein:ActRIIB heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-90 of SEQ ID NO: 521 and ends at any one of amino acids 157-233 of SEQ ID NO: 521. In some embodiments, the Cryptic protein:ActRIIB heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-30 of SEQ ID NO: 525 and ends at any one of amino acids 82-191 of SEQ ID NO: 525. In some embodiments, the Cryptic protein:ActRIIB heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-30 of SEQ ID NO: 529, and ends at any one of amino acids 82-148 of SEQ ID NO: 529. In some embodiments, a Cryptic protein:ActRIIB heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 1, 2, 3, 4, 5, and 6. In some embodiments, a Cryptic protein:ActRIIB heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids 20-29 of SEQ ID NO: 1 and ends at a position corresponding to any one of amino acids 109-134 of SEQ ID NO: 1. In some embodiments, a Cryptic protein:ActRIIB heteromultimer comprises an ActRIIB polypeptide wherein the amino acid position corresponding to L79 of SEQ ID NO: 1 is not an acidic amino acid. In certain preferred embodiments, Cryptic protein:ActRIIB heteromultimers are soluble. In some embodiments, a Cryptic protein:ActRIIB heteromultimer of the disclosure binds to one or more TGF-beta superfamily ligands (e.g., binds to one or more TGF-beta superfamily ligands with a K_{D} of at least 1×10⁻⁷). In some embodiments, a Cryptic protein:ActRIIB heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a Cryptic protein:ActRIIB heteromultimer of the disclosure has a different TGF-beta ligand binding and/or inhibition profile (specificity) compared to a corresponding homomultimer (e.g., Cryptic protein and ActRIIB homomultimers). In some embodiments, a Cryptic protein:ActRIIB heteromultimer of the disclosure is a heterodimer.

In certain aspects, the disclosure relates to heteromultimers that comprise at least one Cryptic protein polypeptide, which includes fragments, functional variants, and modified forms thereof, and at least one TGFBRII polypeptide, which includes fragments, functional variants, and modified forms thereof. In some embodiments, the Cryptic protein:TGFBRII heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 521, 522, 525, 526, 529, or 530. In some embodiments, the Cryptic protein:TGFBRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-90 of SEQ ID NO: 521 and ends at any one of amino acids 157-233 of SEQ ID NO: 521. In some embodiments, the Cryptic protein:TGFBRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-30 of SEQ ID NO: 525 and ends at any one of amino acids 82-191 of SEQ ID NO: 525. In some embodiments, the Cryptic protein:TGFBRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-30 of SEQ ID NO: 529, and ends at any one of amino acids 82-148 of SEQ ID NO: 529. In some embodiments, a Cryptic protein:TGFBRII heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 42, 43, 67, or 68. In some embodiments, a Cryptic protein:TGFBRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 23-51 of SEQ ID NO: 42 and ends at any one of amino acids 143-166 of SEQ ID NO: 42. In some embodiments, a Cryptic protein:TGFBRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 23-44 of SEQ ID NO: 67 and ends at any one of amino acids 168-191 of SEQ ID NO: 67. In certain preferred embodiments, Cryptic protein:TGFBRII heteromultimers are soluble. In some embodiments, a Cryptic protein:TGFBRII heteromultimer of the disclosure binds to one or more TGF-beta superfamily ligands (e.g., binds to one or more TGF-beta superfamily ligands with a K_{D} of at least 1×10⁻⁷). In some embodiments, a Cryptic protein:TGFBRII heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a Cryptic protein:TGFBRII heteromultimer of the disclosure has a different TGF-beta ligand binding and/or inhibition profile (specificity) compared to a corresponding homomultimer (e.g., Cryptic protein and TGFBRII homomultimers). In some embodiments, a Cryptic protein:TGFBRII heteromultimer of the disclosure is a heterodimer.

In certain aspects, the disclosure relates to heteromultimers that comprise at least one Cryptic protein polypeptide, which includes fragments, functional variants, and modified forms thereof, and at least one BMPRII polypeptide, which includes fragments, functional variants, and modified forms thereof. In some embodiments, the Cryptic protein:BMPRII heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 521, 522, 525, 526, 529, or 530. In some embodiments, the Cryptic protein:BMPRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-90 of SEQ ID NO: 521 and ends at any one of amino acids 157-233 of SEQ ID NO: 521. In some embodiments, the Cryptic protein:BMPRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-30 of SEQ ID NO: 525 and ends at any one of amino acids 82-191 of SEQ ID NO: 525. In some embodiments, the Cryptic protein:BMPRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-30 of SEQ ID NO: 529, and ends at any one of amino acids 82-148 of SEQ ID NO: 529. In some embodiments, a Cryptic protein:BMPRII heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 46, 47, 71, or 72. In some embodiments, a Cryptic protein:BMPRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 27-34 of SEQ ID NO: 46 and ends at any one of amino acids 123-150 of SEQ ID NO: 46. In some embodiments, a Cryptic protein:BMPRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 27-34 of SEQ ID NO: 71 and ends at any one of amino acids 123-150 of SEQ ID NO: 71. In certain preferred embodiments, Cryptic protein:BMPRII heteromultimers are soluble. In some embodiments, a Cryptic protein:BMPRII heteromultimer of the disclosure binds to one or more TGF-beta superfamily ligands (e.g., binds to one or more TGF-beta superfamily ligands with a K_{D} of at least 1×10⁻⁷). In some embodiments, a Cryptic protein:BMPRII heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a Cryptic protein:BMPRII heteromultimer of the disclosure has a different TGF-beta ligand binding and/or inhibition profile (specificity) compared to a corresponding homomultimer (e.g., Cryptic protein and BMPRII homomultimers). In some embodiments, a Cryptic protein:BMPRII heteromultimer of the disclosure is a heterodimer.

In certain aspects, the disclosure relates to heteromultimers that comprise at least one Cryptic protein polypeptide, which includes fragments, functional variants, and modified forms thereof, and at least one MISRII polypeptide, which includes fragments, functional variants, and modified forms thereof. In some embodiments, the Cryptic protein:MISRII heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 521, 522, 525, 526, 529, or 530. In some embodiments, the Cryptic protein:MISRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-90 of SEQ ID NO: 521 and ends at any one of amino acids 157-233 of SEQ ID NO: 521. In some embodiments, the Cryptic protein:MISRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-30 of SEQ ID NO: 525 and ends at any one of amino acids 82-191 of SEQ ID NO: 525. In some embodiments, the Cryptic protein:MISRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-30 of SEQ ID NO: 529, and ends at any one of amino acids 82-148 of SEQ ID NO: 529. In some embodiments, a Cryptic protein:MISRII heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 50, 51, 75, 76, 79, or 80. In some embodiments, a Cryptic protein:MISRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 17-24 of SEQ ID NO: 50 and ends at any one of amino acids 116-149 of SEQ ID NO: 50. In some embodiments, a Cryptic protein:MISRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 17-24 of SEQ ID NO: 75 and ends at any one of amino acids 116-149 of SEQ ID NO: 75. In some embodiments, a Cryptic protein:MISRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 17-24 of SEQ ID NO: 50 and ends at any one of amino acids 116-149 of SEQ ID NO: 79. In certain preferred embodiments, Cryptic protein:MISRII heteromultimers are soluble. In some embodiments, a Cryptic protein:MISRII heteromultimer of the disclosure binds to one or more TGF-beta superfamily ligands (e.g., binds to one or more TGF-beta superfamily ligands with a K_{D} of at least 1×10⁻⁷). In some embodiments, a Cryptic protein:MISRII heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a Cryptic protein:MISRII heteromultimer of the disclosure has a different TGF-beta ligand binding and/or inhibition profile (specificity) compared to a corresponding homomultimer (e.g., Cryptic protein and MISRII homomultimers). In some embodiments, a Cryptic protein:MISRII heteromultimer of the disclosure is a heterodimer.

In certain aspects, the disclosure relates to heteromultimers that comprise at least one Cryptic family protein 1B polypeptide, which includes fragments, functional variants, and modified forms thereof, and at least one ActRIIA polypeptide, which includes fragments, functional variants, and modified forms thereof. In some embodiments, the Cryptic family protein 1B:ActRIIA heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 533 or 534. In some embodiments, the Cryptic family protein 1B:ActRIIA heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-30 of SEQ ID NO: 533 and ends at any one of amino acids 82-223 of SEQ ID NO: 533. In some embodiments, a Cryptic family protein 1B:ActRIIA heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 9, 10, and 11. In some embodiments, a Cryptic family protein 1B:ActRIIA heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 21-30 of SEQ ID NO: 9, and ends at any one of amino acids 110-135 of SEQ ID NO: 9. In certain preferred embodiments, Cryptic family protein 1B:ActRIIA heteromultimers are soluble. In some embodiments, a Cryptic family protein 1B:ActRIIA heteromultimer of the disclosure binds to one or more TGF-beta superfamily ligands (e.g., binds to one or more TGF-beta superfamily ligands with a K_{D} of at least 1×10⁻⁷). In some embodiments, a Cryptic family protein 1B:ActRIIA heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a Cryptic family protein 1B:ActRIIA heteromultimer of the disclosure has a different TGF-beta ligand binding and/or inhibition profile (specificity) compared to a corresponding homomultimer (e.g., Cryptic family protein 1B and ActRIIA homomultimers). In some embodiments, a Cryptic family protein 1B:ActRIIA heteromultimer of the disclosure is a heterodimer.

In certain aspects, the disclosure relates to heteromultimers that comprise at least one Cryptic family protein 1B polypeptide, which includes fragments, functional variants, and modified forms thereof, and at least one ActRIIB polypeptide, which includes fragments, functional variants, and modified forms thereof. In some embodiments, the Cryptic family protein 1B:ActRIIB heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 533 or 534. In some embodiments, the Cryptic family protein 1B:ActRIIB heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-30 of SEQ ID NO: 533 and ends at any one of amino acids 82-223 of SEQ ID NO: 533. In some embodiments, a Cryptic family protein 1B:ActRIIB heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 1, 2, 3, 4, 5, and 6. In some embodiments, a Cryptic family protein 1B:ActRIIB heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids 20-29 of SEQ ID NO: 1 and ends at a position corresponding to any one of amino acids 109-134 of SEQ ID NO: 1. In some embodiments, a Cryptic family protein 1B:ActRIIB heteromultimer comprises an ActRIIB polypeptide wherein the amino acid position corresponding to L79 of SEQ ID NO: 1 is not an acidic amino acid. In certain preferred embodiments, Cryptic family protein 1B:ActRIIB heteromultimers are soluble. In some embodiments, a Cryptic family protein 1B:ActRIIB heteromultimer of the disclosure binds to one or more TGF-beta superfamily ligands (e.g., binds to one or more TGF-beta superfamily ligands with a K_{D} of at least 1×10⁻⁷). In some embodiments, a Cryptic family protein 1B:ActRIIB heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a Cryptic family protein 1B:ActRIIB heteromultimer of the disclosure has a different TGF-beta ligand binding and/or inhibition profile (specificity) compared to a corresponding homomultimer (e.g., Cryptic family protein 1B and ActRIIB homomultimers). In some embodiments, a Cryptic family protein 1B:ActRIIB heteromultimer of the disclosure is a heterodimer.

In certain aspects, the disclosure relates to heteromultimers that comprise at least one Cryptic family protein 1B polypeptide, which includes fragments, functional variants, and modified forms thereof, and at least one TGFBRII polypeptide, which includes fragments, functional variants, and modified forms thereof. In some embodiments, the Cryptic family protein 1B:TGFBRII heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 533 or 534. In some embodiments, the Cryptic family protein 1B:TGFBRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-30 of SEQ ID NO: 533 and ends at any one of amino acids 82-223 of SEQ ID NO: 533. In some embodiments, a Cryptic family protein 1B:TGFBRII heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 42, 43, 67, or 68. In some embodiments, a Cryptic family protein 1B:TGFBRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 23-51 of SEQ ID NO: 42 and ends at any one of amino acids 143-166 of SEQ ID NO: 42. In some embodiments, a Cryptic family protein 1B:TGFBRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 23-44 of SEQ ID NO: 67 and ends at any one of amino acids 168-191 of SEQ ID NO: 67. In certain preferred embodiments, Cryptic family protein 1B:TGFBRII heteromultimers are soluble. In some embodiments, a Cryptic family protein 1B:TGFBRII heteromultimer of the disclosure binds to one or more TGF-beta superfamily ligands (e.g., binds to one or more TGF-beta superfamily ligands with a K_{D} of at least 1×10⁻⁷). In some embodiments, a Cryptic family protein 1B:TGFBRII heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a Cryptic family protein 1B:TGFBRII heteromultimer of the disclosure has a different TGF-beta ligand binding and/or inhibition profile (specificity) compared to a corresponding homomultimer (e.g., Cryptic family protein 1B and TGFBRII homomultimers). In some embodiments, a Cryptic family protein 1B:TGFBRII heteromultimer of the disclosure is a heterodimer.

In certain aspects, the disclosure relates to heteromultimers that comprise at least one Cryptic family protein 1B polypeptide, which includes fragments, functional variants, and modified forms thereof, and at least one BMPRII polypeptide, which includes fragments, functional variants, and modified forms thereof. In some embodiments, the Cryptic family protein 1B:BMPRII heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 533 or 534. In some embodiments, the Cryptic family protein 1B:BMPRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-30 of SEQ ID NO: 533 and ends at any one of amino acids 82-223 of SEQ ID NO: 533. In some embodiments, a Cryptic family protein 1B:BMPRII heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 46, 47, 71, or 72. In some embodiments, a Cryptic family protein 1B:BMPRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 27-34 of SEQ ID NO: 46 and ends at any one of amino acids 123-150 of SEQ ID NO: 46. In some embodiments, a Cryptic family protein 1B:BMPRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 27-34 of SEQ ID NO: 71 and ends at any one of amino acids 123-150 of SEQ ID NO: 71. In certain preferred embodiments, Cryptic family protein 1B:BMPRII heteromultimers are soluble. In some embodiments, a Cryptic family protein 1B:BMPRII heteromultimer of the disclosure binds to one or more TGF-beta superfamily ligands (e.g., binds to one or more TGF-beta superfamily ligands with a K_{D} of at least 1×10⁻⁷). In some embodiments, a Cryptic family protein 1B:BMPRII heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a Cryptic family protein 1B:BMPRII heteromultimer of the disclosure has a different TGF-beta ligand binding and/or inhibition profile (specificity) compared to a corresponding homomultimer (e.g., Cryptic family protein 1B and BMPRII homomultimers). In some embodiments, a Cryptic family protein 1B:BMPRII heteromultimer of the disclosure is a heterodimer.

In certain aspects, the disclosure relates to heteromultimers that comprise at least one Cryptic family protein 1B polypeptide, which includes fragments, functional variants, and modified forms thereof, and at least one MISRII polypeptide, which includes fragments, functional variants, and modified forms thereof. In some embodiments, the Cryptic family protein 1B:MISRII heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 533 or 534. In some embodiments, the Cryptic family protein 1B:MISRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-30 of SEQ ID NO: 533 and ends at any one of amino acids 82-223 of SEQ ID NO: 533. In some embodiments, a Cryptic family protein 1B:MISRII heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 50, 51, 75, 76, 79, or 80. In some embodiments, a Cryptic family protein 1B:MISRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 17-24 of SEQ ID NO: 50 and ends at any one of amino acids 116-149 of SEQ ID NO: 50. In some embodiments, a Cryptic family protein 1B:MISRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 17-24 of SEQ ID NO: 75 and ends at any one of amino acids 116-149 of SEQ ID NO: 75. In some embodiments, a Cryptic family protein 1B:MISRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 17-24 of SEQ ID NO: 50 and ends at any one of amino acids 116-149 of SEQ ID NO: 79. In certain preferred embodiments, Cryptic family protein 1B:MISRII heteromultimers are soluble. In some embodiments, a Cryptic family protein 1B:MISRII heteromultimer of the disclosure binds to one or more TGF-beta superfamily ligands (e.g., binds to one or more TGF-beta superfamily ligands with a K_{D} of at least 1×10⁻⁷). In some embodiments, a Cryptic family protein 1B:MISRII heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a Cryptic family protein 1B:MISRII heteromultimer of the disclosure has a different TGF-beta ligand binding and/or inhibition profile (specificity) compared to a corresponding homomultimer (e.g., Cryptic family protein 1B and MISRII homomultimers). In some embodiments, a Cryptic family protein 1B:MISRII heteromultimer of the disclosure is a heterodimer.

In certain aspects, the disclosure relates to heteromultimers that comprise at least one Crim1 polypeptide, which includes fragments, functional variants, and modified forms thereof, and at least one ActRIIA polypeptide, which includes fragments, functional variants, and modified forms thereof. In some embodiments, the Crim1:ActRIIA heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 537 or 538. In some embodiments, the Crim1:ActRIIA heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 35-37 of SEQ ID NO: 537 and ends at any one of amino acids 873-939 of SEQ ID NO: 537. In some embodiments, a Crim1:ActRIIA heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 9, 10, and 11. In some embodiments, a Crim1:ActRIIA heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 21-30 of SEQ ID NO: 9, and ends at any one of amino acids 110-135 of SEQ ID NO: 9. In certain preferred embodiments, Crim1:ActRIIA heteromultimers are soluble. In some embodiments, a Crim1:ActRIIA heteromultimer of the disclosure binds to one or more TGF-beta superfamily ligands (e.g., binds to one or more TGF-beta superfamily ligands with a K_{D} of at least 1×10⁻⁷). In some embodiments, a Crim1:ActRIIA heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a Crim1:ActRIIA heteromultimer of the disclosure has a different TGF-beta ligand binding and/or inhibition profile (specificity) compared to a corresponding homomultimer (e.g., Crim1 and ActRIIA homomultimers). In some embodiments, a Crim1:ActRIIA heteromultimer of the disclosure is a heterodimer.

In certain aspects, the disclosure relates to heteromultimers that comprise at least one Crim1 polypeptide, which includes fragments, functional variants, and modified forms thereof, and at least one ActRIIB polypeptide, which includes fragments, functional variants, and modified forms thereof. In some embodiments, the Crim1:ActRIIB heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 537 or 538. In some embodiments, the Crim1:ActRIIB heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 35-37 of SEQ ID NO: 537 and ends at any one of amino acids 873-939 of SEQ ID NO: 537. In some embodiments, a Crim1:ActRIIB heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 1, 2, 3, 4, 5, and 6. In some embodiments, a Crim1:ActRIIB heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids 20-29 of SEQ ID NO: 1 and ends at a position corresponding to any one of amino acids 109-134 of SEQ ID NO: 1. In some embodiments, a Crim1:ActRIIB heteromultimer comprises an ActRIIB polypeptide wherein the amino acid position corresponding to L79 of SEQ ID NO: 1 is not an acidic amino acid. In certain preferred embodiments, Crim1:ActRIIB heteromultimers are soluble. In some embodiments, a Crim1:ActRIIB heteromultimer of the disclosure binds to one or more TGF-beta superfamily ligands (e.g., binds to one or more TGF-beta superfamily ligands with a K_{D} of at least 1×10⁻⁷). In some embodiments, a Crim1:ActRIIB heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a Crim1:ActRIIB heteromultimer of the disclosure has a different TGF-beta ligand binding and/or inhibition profile (specificity) compared to a corresponding homomultimer (e.g., Crim1 and ActRIIB homomultimers). In some embodiments, a Crim1:ActRIIB heteromultimer of the disclosure is a heterodimer.

In certain aspects, the disclosure relates to heteromultimers that comprise at least one Crim1 polypeptide, which includes fragments, functional variants, and modified forms thereof, and at least one TGFBRII polypeptide, which includes fragments, functional variants, and modified forms thereof. In some embodiments, the Crim1:TGFBRII heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 537 or 538. In some embodiments, the Crim1:TGFBRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 35-37 of SEQ ID NO: 537 and ends at any one of amino acids 873-939 of SEQ ID NO: 537. In some embodiments, a Crim1:TGFBRII heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 42, 43, 67, or 68. In some embodiments, a Crim1:TGFBRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 23-51 of SEQ ID NO: 42 and ends at any one of amino acids 143-166 of SEQ ID NO: 42. In some embodiments, a Crim1:TGFBRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 23-44 of SEQ ID NO: 67 and ends at any one of amino acids 168-191 of SEQ ID NO: 67. In certain preferred embodiments, Crim1:TGFBRII heteromultimers are soluble. In some embodiments, a Crim1:TGFBRII heteromultimer of the disclosure binds to one or more TGF-beta superfamily ligands (e.g., binds to one or more TGF-beta superfamily ligands with a K_{D} of at least 1×10⁻⁷). In some embodiments, a Crim1:TGFBRII heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a Crim1:TGFBRII heteromultimer of the disclosure has a different TGF-beta ligand binding and/or inhibition profile (specificity) compared to a corresponding homomultimer (e.g., Crim1 and TGFBRII homomultimers). In some embodiments, a Crim1:TGFBRII heteromultimer of the disclosure is a heterodimer.

In certain aspects, the disclosure relates to heteromultimers that comprise at least one Crim1 polypeptide, which includes fragments, functional variants, and modified forms thereof, and at least one BMPRII polypeptide, which includes fragments, functional variants, and modified forms thereof. In some embodiments, the Crim1:BMPRII heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 537 or 538. In some embodiments, the Crim1:BMPRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 35-37 of SEQ ID NO: 537 and ends at any one of amino acids 873-939 of SEQ ID NO: 537. In some embodiments, a Crim1:BMPRII heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 46, 47, 71, or 72. In some embodiments, a Crim1:BMPRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 27-34 of SEQ ID NO: 46 and ends at any one of amino acids 123-150 of SEQ ID NO: 46. In some embodiments, a Crim1:BMPRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 27-34 of SEQ ID NO: 71 and ends at any one of amino acids 123-150 of SEQ ID NO: 71. In certain preferred embodiments, Crim1:BMPRII heteromultimers are soluble. In some embodiments, a Crim1:BMPRII heteromultimer of the disclosure binds to one or more TGF-beta superfamily ligands (e.g., binds to one or more TGF-beta superfamily ligands with a K_{D} of at least 1×10⁻⁷). In some embodiments, a Crim1:BMPRII heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a Crim1:BMPRII heteromultimer of the disclosure has a different TGF-beta ligand binding and/or inhibition profile (specificity) compared to a corresponding homomultimer (e.g., Crim1 and BMPRII homomultimers). In some embodiments, a Crim1:BMPRII heteromultimer of the disclosure is a heterodimer.

In certain aspects, the disclosure relates to heteromultimers that comprise at least one Crim1 polypeptide, which includes fragments, functional variants, and modified forms thereof, and at least one MISRII polypeptide, which includes fragments, functional variants, and modified forms thereof. In some embodiments, the Crim1:MISRII heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 537 or 538. In some embodiments, the Crim1:MISRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 35-37 of SEQ ID NO: 537 and ends at any one of amino acids 873-939 of SEQ ID NO: 537. In some embodiments, a Crim1:MISRII heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 50, 51, 75, 76, 79, or 80. In some embodiments, a Crim1:MISRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 17-24 of SEQ ID NO: 50 and ends at any one of amino acids 116-149 of SEQ ID NO: 50. In some embodiments, a Crim1:MISRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 17-24 of SEQ ID NO: 75 and ends at any one of amino acids 116-149 of SEQ ID NO: 75. In some embodiments, a Crim1:MISRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 17-24 of SEQ ID NO: 50 and ends at any one of amino acids 116-149 of SEQ ID NO: 79. In certain preferred embodiments, Crim1:MISRII heteromultimers are soluble. In some embodiments, a Crim1:MISRII heteromultimer of the disclosure binds to one or more TGF-beta superfamily ligands (e.g., binds to one or more TGF-beta superfamily ligands with a K_{D} of at least 1×10⁻⁷). In some embodiments, a Crim1:MISRII heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a Crim1:MISRII heteromultimer of the disclosure has a different TGF-beta ligand binding and/or inhibition profile (specificity) compared to a corresponding homomultimer (e.g., Crim1 and MISRII homomultimers). In some embodiments, a Crim1:MISRII heteromultimer of the disclosure is a heterodimer.

In certain aspects, the disclosure relates to heteromultimers that comprise at least one Crim2 polypeptide, which includes fragments, functional variants, and modified forms thereof, and at least one ActRIIA polypeptide, which includes fragments, functional variants, and modified forms thereof. In some embodiments, the Crim2:ActRIIA heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 541, 542, 545, or 546. In some embodiments, the Crim2:ActRIIA heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-138 of SEQ ID NO: 541 and ends at any one of amino acids 1298-1503 of SEQ ID NO: 541. In some embodiments, the Crim2:ActRIIA heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 24-138 of SEQ ID NO: 545 and ends at any one of amino acids 539-814 of SEQ ID NO: 545. In some embodiments, a Crim2:ActRIIA heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 9, 10, and 11. In some embodiments, a Crim2:ActRIIA heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 21-30 of SEQ ID NO: 9, and ends at any one of amino acids 110-135 of SEQ ID NO: 9. In certain preferred embodiments, Crim2:ActRIIA heteromultimers are soluble. In some embodiments, a Crim2:ActRIIA heteromultimer of the disclosure binds to one or more TGF-beta superfamily ligands (e.g., binds to one or more TGF-beta superfamily ligands with a K_{D} of at least 1×10⁻⁷). In some embodiments, a Crim2:ActRIIA heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a Crim2:ActRIIA heteromultimer of the disclosure has a different TGF-beta ligand binding and/or inhibition profile (specificity) compared to a corresponding homomultimer (e.g., Crim2 and ActRIIA homomultimers). In some embodiments, a Crim2:ActRIIA heteromultimer of the disclosure is a heterodimer.

In certain aspects, the disclosure relates to heteromultimers that comprise at least one Crim2 polypeptide, which includes fragments, functional variants, and modified forms thereof, and at least one ActRIIB polypeptide, which includes fragments, functional variants, and modified forms thereof. In some embodiments, the Crim2:ActRIIB heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 541, 542, 545, or 546. In some embodiments, the Crim2:ActRIIB heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-138 of SEQ ID NO: 541 and ends at any one of amino acids 1298-1503 of SEQ ID NO: 541. In some embodiments, the Crim2:ActRIIB heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 24-138 of SEQ ID NO: 545 and ends at any one of amino acids 539-814 of SEQ ID NO: 545. In some embodiments, a Crim2:ActRIIB heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 1, 2, 3, 4, 5, and 6. In some embodiments, a Crim2:ActRIIB heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids 20-29 of SEQ ID NO: 1 and ends at a position corresponding to any one of amino acids 109-134 of SEQ ID NO: 1. In some embodiments, a Crim2:ActRIIB heteromultimer comprises an ActRIIB polypeptide wherein the amino acid position corresponding to L79 of SEQ ID NO: 1 is not an acidic amino acid. In certain preferred embodiments, Crim2:ActRIIB heteromultimers are soluble. In some embodiments, a Crim2:ActRIIB heteromultimer of the disclosure binds to one or more TGF-beta superfamily ligands (e.g., binds to one or more TGF-beta superfamily ligands with a K_{D} of at least 1×10⁻⁷). In some embodiments, a Crim2:ActRIIB heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a Crim2:ActRIIB heteromultimer of the disclosure has a different TGF-beta ligand binding and/or inhibition profile (specificity) compared to a corresponding homomultimer (e.g., Crim2 and ActRIIB homomultimers). In some embodiments, a Crim2:ActRIIB heteromultimer of the disclosure is a heterodimer.

In certain aspects, the disclosure relates to heteromultimers that comprise at least one Crim2 polypeptide, which includes fragments, functional variants, and modified forms thereof, and at least one TGFBRII polypeptide, which includes fragments, functional variants, and modified forms thereof. In some embodiments, the Crim2:TGFBRII heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 541, 542, 545, or 546. In some embodiments, the Crim2:TGFBRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-138 of SEQ ID NO: 541 and ends at any one of amino acids 1298-1503 of SEQ ID NO: 541. In some embodiments, the Crim2:TGFBRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 24-138 of SEQ ID NO: 545 and ends at any one of amino acids 539-814 of SEQ ID NO: 545. In some embodiments, a Crim2:TGFBRII heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 42, 43, 67, or 68. In some embodiments, a Crim2:TGFBRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 23-51 of SEQ ID NO: 42 and ends at any one of amino acids 143-166 of SEQ ID NO: 42. In some embodiments, a Crim2:TGFBRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 23-44 of SEQ ID NO: 67 and ends at any one of amino acids 168-191 of SEQ ID NO: 67. In certain preferred embodiments, Crim2:TGFBRII heteromultimers are soluble. In some embodiments, a Crim2:TGFBRII heteromultimer of the disclosure binds to one or more TGF-beta superfamily ligands (e.g., binds to one or more TGF-beta superfamily ligands with a K_{D} of at least 1×10⁻⁷). In some embodiments, a Crim2:TGFBRII heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a Crim2:TGFBRII heteromultimer of the disclosure has a different TGF-beta ligand binding and/or inhibition profile (specificity) compared to a corresponding homomultimer (e.g., Crim2 and TGFBRII homomultimers). In some embodiments, a Crim2:TGFBRII heteromultimer of the disclosure is a heterodimer.

In certain aspects, the disclosure relates to heteromultimers that comprise at least one Crim2 polypeptide, which includes fragments, functional variants, and modified forms thereof, and at least one BMPRII polypeptide, which includes fragments, functional variants, and modified forms thereof. In some embodiments, the Crim2:BMPRII heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 541, 542, 545, or 546. In some embodiments, the Crim2:BMPRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-138 of SEQ ID NO: 541 and ends at any one of amino acids 1298-1503 of SEQ ID NO: 541. In some embodiments, the Crim2:BMPRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 24-138 of SEQ ID NO: 545 and ends at any one of amino acids 539-814 of SEQ ID NO: 545. In some embodiments, a Crim2:BMPRII heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 46, 47, 71, or 72. In some embodiments, a Crim2:BMPRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 27-34 of SEQ ID NO: 46 and ends at any one of amino acids 123-150 of SEQ ID NO: 46. In some embodiments, a Crim2:BMPRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 27-34 of SEQ ID NO: 71 and ends at any one of amino acids 123-150 of SEQ ID NO: 71. In certain preferred embodiments, Crim2:BMPRII heteromultimers are soluble. In some embodiments, a Crim2:BMPRII heteromultimer of the disclosure binds to one or more TGF-beta superfamily ligands (e.g., binds to one or more TGF-beta superfamily ligands with a K_{D} of at least 1×10⁻⁷). In some embodiments, a Crim2:BMPRII heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a Crim2:BMPRII heteromultimer of the disclosure has a different TGF-beta ligand binding and/or inhibition profile (specificity) compared to a corresponding homomultimer (e.g., Crim2 and BMPRII homomultimers). In some embodiments, a Crim2:BMPRII heteromultimer of the disclosure is a heterodimer.

In certain aspects, the disclosure relates to heteromultimers that comprise at least one Crim2 polypeptide, which includes fragments, functional variants, and modified forms thereof, and at least one MISRII polypeptide, which includes fragments, functional variants, and modified forms thereof. In some embodiments, the Crim2:MISRII heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 541, 542, 545, or 546. In some embodiments, the Crim2:MISRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-138 of SEQ ID NO: 541 and ends at any one of amino acids 1298-1503 of SEQ ID NO: 541. In some embodiments, the Crim2:MISRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 24-138 of SEQ ID NO: 545 and ends at any one of amino acids 539-814 of SEQ ID NO: 545. In some embodiments, a Crim2:MISRII heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 50, 51, 75, 76, 79, or 80. In some embodiments, a Crim2:MISRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 17-24 of SEQ ID NO: 50 and ends at any one of amino acids 116-149 of SEQ ID NO: 50. In some embodiments, a Crim2:MISRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 17-24 of SEQ ID NO: 75 and ends at any one of amino acids 116-149 of SEQ ID NO: 75. In some embodiments, a Crim2:MISRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 17-24 of SEQ ID NO: 50 and ends at any one of amino acids 116-149 of SEQ ID NO: 79. In certain preferred embodiments, Crim2:MISRII heteromultimers are soluble. In some embodiments, a Crim2:MISRII heteromultimer of the disclosure binds to one or more TGF-beta superfamily ligands (e.g., binds to one or more TGF-beta superfamily ligands with a K_{D} of at least 1×10⁻⁷). In some embodiments, a Crim2:MISRII heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a Crim2:MISRII heteromultimer of the disclosure has a different TGF-beta ligand binding and/or inhibition profile (specificity) compared to a corresponding homomultimer (e.g., Crim2 and MISRII homomultimers). In some embodiments, a Crim2:MISRII heteromultimer of the disclosure is a heterodimer.

In certain aspects, the disclosure relates to heteromultimers that comprise at least one BAMBI polypeptide, which includes fragments, functional variants, and modified forms thereof, and at least one ActRIIA polypeptide, which includes fragments, functional variants, and modified forms thereof. In some embodiments, the BAMBI:ActRIIA heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 549 or 550. In some embodiments, the BAMBI:ActRIIA heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 21-30 of SEQ ID NO: 549 and ends at any one of amino acids 104-152 of SEQ ID NO: 549. In some embodiments, a BAMBI:ActRIIA heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 9, 10, and 11. In some embodiments, a BAMBI:ActRIIA heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 21-30 of SEQ ID NO: 9, and ends at any one of amino acids 110-135 of SEQ ID NO: 9. In certain preferred embodiments, BAMBI:ActRIIA heteromultimers are soluble. In some embodiments, a BAMBI:ActRIIA heteromultimer of the disclosure binds to one or more TGF-beta superfamily ligands (e.g., binds to one or more TGF-beta superfamily ligands with a K_{D} of at least 1×10⁻⁷). In some embodiments, a BAMBI:ActRIIA heteromultimer of the disclosure inhibits one or more TGF-beta superfamily ligands (e.g., inhibits Smad signaling).

Heteromultimer-ligand binding and inhibition may be determined using a variety of assays including, for example, those described herein (e.g., in vitro binding and/or cell-based signaling assays). In some embodiments, a BAMBI:ActRIIA heteromultimer of the disclosure has a different TGF-beta ligand binding and/or inhibition profile (specificity) compared to a corresponding homomultimer (e.g., BAMBI and ActRIIA homomultimers). In some embodiments, a BAMBI:ActRIIA heteromultimer of the disclosure is a heterodimer.

In certain aspects, the disclosure relates to heteromultimers that comprise at least one BAMBI polypeptide, which includes fragments, functional variants, and modified forms thereof, and at least one ActRIIB polypeptide, which includes fragments, functional variants, and modified forms thereof. In some embodiments, the BAMBI:ActRIIB heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 549 or 550. In some embodiments, the BAMBI:ActRIIB heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 21-30 of SEQ ID NO: 549 and ends at any one of amino acids 104-152 of SEQ ID NO: 549. In some embodiments, a BAMBI:ActRIIB heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 1, 2, 3, 4, 5, and 6. In some embodiments, a BAMBI:ActRIIB heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids 20-29 of SEQ ID NO: 1 and ends at a position corresponding to any one of amino acids 109-134 of SEQ ID NO: 1. In some embodiments, a BAMBI:ActRIIB heteromultimer comprises an ActRIIB polypeptide wherein the amino acid position corresponding to L79 of SEQ ID NO: 1 is not an acidic amino acid. In certain preferred embodiments, BAMBI:ActRIIB heteromultimers are soluble. In some embodiments, a BAMBI:ActRIIB heteromultimer of the disclosure binds to one or more TGF-beta superfamily ligands (e.g., binds to one or more TGF-beta superfamily ligands with a K_{D} of at least 1×10⁻⁷). In some embodiments, a BAMBI:ActRIIB heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a BAMBI:ActRIIB heteromultimer of the disclosure has a different TGF-beta ligand binding and/or inhibition profile (specificity) compared to a corresponding homomultimer (e.g., BAMBI and ActRIIB homomultimers). In some embodiments, a BAMBI:ActRIIB heteromultimer of the disclosure is a heterodimer.

In certain aspects, the disclosure relates to heteromultimers that comprise at least one BAMBI polypeptide, which includes fragments, functional variants, and modified forms thereof, and at least one TGFBRII polypeptide, which includes fragments, functional variants, and modified forms thereof. In some embodiments, the BAMBI:TGFBRII heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 549 or 550. In some embodiments, the BAMBI:TGFBRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 21-30 of SEQ ID NO: 549 and ends at any one of amino acids 104-152 of SEQ ID NO: 549. In some embodiments, a BAMBI:TGFBRII heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 42, 43, 67, or 68. In some embodiments, a BAMBI:TGFBRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 23-51 of SEQ ID NO: 42 and ends at any one of amino acids 143-166 of SEQ ID NO: 42. In some embodiments, a BAMBI:TGFBRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 23-44 of SEQ ID NO: 67 and ends at any one of amino acids 168-191 of SEQ ID NO: 67. In certain preferred embodiments, BAMBI:TGFBRII heteromultimers are soluble. In some embodiments, a BAMBI:TGFBRII heteromultimer of the disclosure binds to one or more TGF-beta superfamily ligands (e.g., binds to one or more TGF-beta superfamily ligands with a K_{D} of at least 1×10⁻⁷). In some embodiments, a BAMBI:TGFBRII heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a BAMBI:TGFBRII heteromultimer of the disclosure has a different TGF-beta ligand binding and/or inhibition profile (specificity) compared to a corresponding homomultimer (e.g., BAMBI and TGFBRII homomultimers). In some embodiments, a BAMBI:TGFBRII heteromultimer of the disclosure is a heterodimer.

In certain aspects, the disclosure relates to heteromultimers that comprise at least one BAMBI polypeptide, which includes fragments, functional variants, and modified forms thereof, and at least one BMPRII polypeptide, which includes fragments, functional variants, and modified forms thereof. In some embodiments, the BAMBI:BMPRII heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 549 or 550. In some embodiments, the BAMBI:BMPRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 21-30 of SEQ ID NO: 549 and ends at any one of amino acids 104-152 of SEQ ID NO: 549. In some embodiments, a BAMBI:BMPRII heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 46, 47, 71, or 72. In some embodiments, a BAMBI:BMPRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 27-34 of SEQ ID NO: 46 and ends at any one of amino acids 123-150 of SEQ ID NO: 46. In some embodiments, a BAMBI:BMPRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 27-34 of SEQ ID NO: 71 and ends at any one of amino acids 123-150 of SEQ ID NO: 71. In certain preferred embodiments, BAMBI:BMPRII heteromultimers are soluble. In some embodiments, a BAMBI:BMPRII heteromultimer of the disclosure binds to one or more TGF-beta superfamily ligands (e.g., binds to one or more TGF-beta superfamily ligands with a K_{D} of at least 1×10⁻⁷). In some embodiments, a BAMBI:BMPRII heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a BAMBI:BMPRII heteromultimer of the disclosure has a different TGF-beta ligand binding and/or inhibition profile (specificity) compared to a corresponding homomultimer (e.g., BAMBI and BMPRII homomultimers). In some embodiments, a BAMBI:BMPRII heteromultimer of the disclosure is a heterodimer.

In certain aspects, the disclosure relates to heteromultimers that comprise at least one BAMBI polypeptide, which includes fragments, functional variants, and modified forms thereof, and at least one MISRII polypeptide, which includes fragments, functional variants, and modified forms thereof. In some embodiments, the BAMBI:MISRII heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 549 or 550. In some embodiments, the BAMBI:MISRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 21-30 of SEQ ID NO: 549 and ends at any one of amino acids 104-152 of SEQ ID NO: 549. In some embodiments, a BAMBI:MISRII heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 50, 51, 75, 76, 79, or 80. In some embodiments, a BAMBI:MISRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 17-24 of SEQ ID NO: 50 and ends at any one of amino acids 116-149 of SEQ ID NO: 50. In some embodiments, a BAMBI:MISRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 17-24 of SEQ ID NO: 75 and ends at any one of amino acids 116-149 of SEQ ID NO: 75. In some embodiments, a BAMBI:MISRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 17-24 of SEQ ID NO: 50 and ends at any one of amino acids 116-149 of SEQ ID NO: 79. In certain preferred embodiments, BAMBI:MISRII heteromultimers are soluble. In some embodiments, a BAMBI:MISRII heteromultimer of the disclosure binds to one or more TGF-beta superfamily ligands (e.g., binds to one or more TGF-beta superfamily ligands with a K_{D} of at least 1×10⁻⁷). In some embodiments, a BAMBI:MISRII heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a BAMBI:MISRII heteromultimer of the disclosure has a different TGF-beta ligand binding and/or inhibition profile (specificity) compared to a corresponding homomultimer (e.g., BAMBI and MISRII homomultimers). In some embodiments, a BAMBI:MISRII heteromultimer of the disclosure is a heterodimer.

In certain aspects, the disclosure relates to heteromultimers that comprise at least one BMPER polypeptide, which includes fragments, functional variants, and modified forms thereof, and at least one ActRIIA polypeptide, which includes fragments, functional variants, and modified forms thereof. In some embodiments, the BMPER:ActRIIA heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 553 or 554. In some embodiments, the BMPER:ActRIIA heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 39-50 of SEQ ID NO: 553 and ends at any one of amino acids 364-369 of SEQ ID NO: 553. In some embodiments, the BMPER:ActRIIA heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 370-386 of SEQ ID NO: 553 and ends at any one of amino acids 682-685 of SEQ ID NO: 553. In some embodiments, the BMPER:ActRIIA heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 39-50 of SEQ ID NO: 553 and ends at any one of amino acids 682-685 of SEQ ID NO: 553. In some embodiments, the BMPER:ActRIIA heteromultimer comprises a BMPER protein, wherein the BMPER protein is a dimer comprising a first polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 39-50 of SEQ ID NO: 553 and ends at any one of amino acids 364-369 of SEQ ID NO: 553, and second polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 370-386 of SEQ ID NO: 553, and ends at any one of amino acids 682-685 of SEQ ID NO: 553. In some embodiments, the BMPER:ActRIIA heteromultimer comprises a single chain ligand trap that comprises a first BMPER polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 39-50 of SEQ ID NO: 553 and ends at any one of amino acids 364-369 of SEQ ID NO: 553, and second BMPER polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 370-386 of SEQ ID NO: 553 and ends at any one of amino acids 682-685 of SEQ ID NO: 553. In some embodiments, a BMPER:ActRIIA heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 9, 10, and 11. In some embodiments, a BMPER:ActRIIA heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 21-30 of SEQ ID NO: 9, and ends at any one of amino acids 110-135 of SEQ ID NO: 9. In certain preferred embodiments, BMPER:ActRIIA heteromultimers are soluble. In some embodiments, a BMPER:ActRIIA heteromultimer of the disclosure binds to one or more TGF-beta superfamily ligands (e.g., binds to one or more TGF-beta superfamily ligands with a K_{D} of at least 1×10⁻⁷). In some embodiments, a BMPER:ActRIIA heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a BMPER:ActRIIA heteromultimer of the disclosure has a different TGF-beta ligand binding and/or inhibition profile (specificity) compared to a corresponding homomultimer (e.g., BMPER and ActRIIA homomultimers). In some embodiments, a BMPER:ActRIIA heteromultimer of the disclosure is a heterodimer.

In certain aspects, the disclosure relates to heteromultimers that comprise at least one BMPER polypeptide, which includes fragments, functional variants, and modified forms thereof, and at least one ActRIIB polypeptide, which includes fragments, functional variants, and modified forms thereof. In some embodiments, the BMPER:ActRIIB heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 553 or 554. In some embodiments, the BMPER:ActRIIB heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 39-50 of SEQ ID NO: 553 and ends at any one of amino acids 364-369 of SEQ ID NO: 553. In some embodiments, the BMPER:ActRIIB heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 370-386 of SEQ ID NO: 553 and ends at any one of amino acids 682-685 of SEQ ID NO: 553. In some embodiments, the BMPER:ActRIIB heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 39-50 of SEQ ID NO: 553 and ends at any one of amino acids 682-685 of SEQ ID NO: 553. In some embodiments, the BMPER:ActRIIB heteromultimer comprises a BMPER protein, wherein the BMPER protein is a dimer comprising a first polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 39-50 of SEQ ID NO: 553 and ends at any one of amino acids 364-369 of SEQ ID NO: 553, and second polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 370-386 of SEQ ID NO: 553, and ends at any one of amino acids 682-685 of SEQ ID NO: 553. In some embodiments, the BMPER:ActRIIB heteromultimer comprises a single chain ligand trap that comprises a first BMPER polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 39-50 of SEQ ID NO: 553 and ends at any one of amino acids 364-369 of SEQ ID NO: 553, and second BMPER polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 370-386 of SEQ ID NO: 553 and ends at any one of amino acids 682-685 of SEQ ID NO: 553. In some embodiments, a BMPER:ActRIIB heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 1, 2, 3, 4, 5, and 6. In some embodiments, a BMPER:ActRIIB heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids 20-29 of SEQ ID NO: 1 and ends at a position corresponding to any one of amino acids 109-134 of SEQ ID NO: 1. In some embodiments, a BMPER:ActRIIB heteromultimer comprises an ActRIIB polypeptide wherein the amino acid position corresponding to L79 of SEQ ID NO: 1 is not an acidic amino acid. In certain preferred embodiments, BMPER:ActRIIB heteromultimers are soluble. In some embodiments, a BMPER:ActRIIB heteromultimer of the disclosure binds to one or more TGF-beta superfamily ligands (e.g., binds to one or more TGF-beta superfamily ligands with a K_{D} of at least 1×10⁻⁷). In some embodiments, a BMPER:ActRIIB heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a BMPER:ActRIIB heteromultimer of the disclosure has a different TGF-beta ligand binding and/or inhibition profile (specificity) compared to a corresponding homomultimer (e.g., BMPER and ActRIIB homomultimers). In some embodiments, a BMPER:ActRIIB heteromultimer of the disclosure is a heterodimer.

In certain aspects, the disclosure relates to heteromultimers that comprise at least one BMPER polypeptide, which includes fragments, functional variants, and modified forms thereof, and at least one TGFBRII polypeptide, which includes fragments, functional variants, and modified forms thereof. In some embodiments, the BMPER:TGFBRII heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 553 or 554. In some embodiments, the BMPER:TGFBRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 39-50 of SEQ ID NO: 553 and ends at any one of amino acids 364-369 of SEQ ID NO: 553. In some embodiments, the BMPER:TGFBRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 370-386 of SEQ ID NO: 553 and ends at any one of amino acids 682-685 of SEQ ID NO: 553. In some embodiments, the BMPER:TGFBRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 39-50 of SEQ ID NO: 553 and ends at any one of amino acids 682-685 of SEQ ID NO: 553. In some embodiments, the BMPER:TGFBRII heteromultimer comprises a BMPER protein, wherein the BMPER protein is a dimer comprising a first polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 39-50 of SEQ ID NO: 553 and ends at any one of amino acids 364-369 of SEQ ID NO: 553, and second polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 370-386 of SEQ ID NO: 553, and ends at any one of amino acids 682-685 of SEQ ID NO: 553. In some embodiments, the BMPER:TGFBRII heteromultimer comprises a single chain ligand trap that comprises a first BMPER polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 39-50 of SEQ ID NO: 553 and ends at any one of amino acids 364-369 of SEQ ID NO: 553, and second BMPER polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 370-386 of SEQ ID NO: 553 and ends at any one of amino acids 682-685 of SEQ ID NO: 553. In some embodiments, a BMPER:TGFBRII heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 42, 43, 67, or 68. In some embodiments, a BMPER:TGFBRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 23-51 of SEQ ID NO: 42 and ends at any one of amino acids 143-166 of SEQ ID NO: 42. In some embodiments, a BMPER:TGFBRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 23-44 of SEQ ID NO: 67 and ends at any one of amino acids 168-191 of SEQ ID NO: 67. In certain preferred embodiments, BMPER:TGFBRII heteromultimers are soluble. In some embodiments, a BMPER:TGFBRII heteromultimer of the disclosure binds to one or more TGF-beta superfamily ligands (e.g., binds to one or more TGF-beta superfamily ligands with a K_{D} of at least 1×10⁻⁷). In some embodiments, a BMPER:TGFBRII heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a BMPER:TGFBRII heteromultimer of the disclosure has a different TGF-beta ligand binding and/or inhibition profile (specificity) compared to a corresponding homomultimer (e.g., BMPER and TGFBRII homomultimers). In some embodiments, a BMPER:TGFBRII heteromultimer of the disclosure is a heterodimer.

In certain aspects, the disclosure relates to heteromultimers that comprise at least one BMPER polypeptide, which includes fragments, functional variants, and modified forms thereof, and at least one BMPRII polypeptide, which includes fragments, functional variants, and modified forms thereof. In some embodiments, the BMPER:BMPRII heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 553 or 554. In some embodiments, the BMPER:BMPRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 39-50 of SEQ ID NO: 553 and ends at any one of amino acids 364-369 of SEQ ID NO: 553. In some embodiments, the BMPER:BMPRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 370-386 of SEQ ID NO: 553 and ends at any one of amino acids 682-685 of SEQ ID NO: 553. In some embodiments, the BMPER:BMPRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 39-50 of SEQ ID NO: 553 and ends at any one of amino acids 682-685 of SEQ ID NO: 553. In some embodiments, the BMPER:BMPRII heteromultimer comprises a BMPER protein, wherein the BMPER protein is a dimer comprising a first polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 39-50 of SEQ ID NO: 553 and ends at any one of amino acids 364-369 of SEQ ID NO: 553, and second polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 370-386 of SEQ ID NO: 553, and ends at any one of amino acids 682-685 of SEQ ID NO: 553. In some embodiments, the BMPER:BMPRII heteromultimer comprises a single chain ligand trap that comprises a first BMPER polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 39-50 of SEQ ID NO: 553 and ends at any one of amino acids 364-369 of SEQ ID NO: 553, and second BMPER polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 370-386 of SEQ ID NO: 553 and ends at any one of amino acids 682-685 of SEQ ID NO: 553. In some embodiments, a BMPER:BMPRII heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 46, 47, 71, or 72. In some embodiments, a BMPER:BMPRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 27-34 of SEQ ID NO: 46 and ends at any one of amino acids 123-150 of SEQ ID NO: 46. In some embodiments, a BMPER:BMPRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 27-34 of SEQ ID NO: 71 and ends at any one of amino acids 123-150 of SEQ ID NO: 71. In certain preferred embodiments, BMPER:BMPRII heteromultimers are soluble. In some embodiments, a BMPER:BMPRII heteromultimer of the disclosure binds to one or more TGF-beta superfamily ligands (e.g., binds to one or more TGF-beta superfamily ligands with a K_{D} of at least 1×10⁻⁷). In some embodiments, a BMPER:BMPRII heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a BMPER:BMPRII heteromultimer of the disclosure has a different TGF-beta ligand binding and/or inhibition profile (specificity) compared to a corresponding homomultimer (e.g., BMPER and BMPRII homomultimers). In some embodiments, a BMPER:BMPRII heteromultimer of the disclosure is a heterodimer.

In certain aspects, the disclosure relates to heteromultimers that comprise at least one BMPER polypeptide, which includes fragments, functional variants, and modified forms thereof, and at least one MISRII polypeptide, which includes fragments, functional variants, and modified forms thereof. In some embodiments, the BMPER:MISRII heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 553 or 554. In some embodiments, the BMPER:MISRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 39-50 of SEQ ID NO: 553 and ends at any one of amino acids 364-369 of SEQ ID NO: 553. In some embodiments, the BMPER:MISRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 370-386 of SEQ ID NO: 553 and ends at any one of amino acids 682-685 of SEQ ID NO: 553. In some embodiments, the BMPER:MISRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 39-50 of SEQ ID NO: 553 and ends at any one of amino acids 682-685 of SEQ ID NO: 553. In some embodiments, the BMPER:MISRII heteromultimer comprises a BMPER protein, wherein the BMPER protein is a dimer comprising a first polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 39-50 of SEQ ID NO: 553 and ends at any one of amino acids 364-369 of SEQ ID NO: 553, and second polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 370-386 of SEQ ID NO: 553, and ends at any one of amino acids 682-685 of SEQ ID NO: 553. In some embodiments, the BMPER:MISRII heteromultimer comprises a single chain ligand trap that comprises a first BMPER polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 39-50 of SEQ ID NO: 553 and ends at any one of amino acids 364-369 of SEQ ID NO: 553, and second BMPER polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 370-386 of SEQ ID NO: 553 and ends at any one of amino acids 682-685 of SEQ ID NO: 553. In some embodiments, a BMPER:MISRII heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 50, 51, 75, 76, 79, or 80. In some embodiments, a BMPER:MISRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 17-24 of SEQ ID NO: 50 and ends at any one of amino acids 116-149 of SEQ ID NO: 50. In some embodiments, a BMPER:MISRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 17-24 of SEQ ID NO: 75 and ends at any one of amino acids 116-149 of SEQ ID NO: 75. In some embodiments, a BMPER:MISRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 17-24 of SEQ ID NO: 50 and ends at any one of amino acids 116-149 of SEQ ID NO: 79. In certain preferred embodiments, BMPER:MISRII heteromultimers are soluble. In some embodiments, a BMPER:MISRII heteromultimer of the disclosure binds to one or more TGF-beta superfamily ligands (e.g., binds to one or more TGF-beta superfamily ligands with a K_{D} of at least 1×10⁻⁷). In some embodiments, a BMPER:MISRII heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a BMPER:MISRII heteromultimer of the disclosure has a different TGF-beta ligand binding and/or inhibition profile (specificity) compared to a corresponding homomultimer (e.g., BMPER and MISRII homomultimers). In some embodiments, a BMPER:MISRII heteromultimer of the disclosure is a heterodimer.

In certain aspects, the disclosure relates to heteromultimers that comprise at least one RGM-A polypeptide, which includes fragments, functional variants, and modified forms thereof, and at least one ActRIIA polypeptide, which includes fragments, functional variants, and modified forms thereof. In some embodiments, the RGM-A:ActRIIA heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 553 or 554. In some embodiments, the RGM-A:ActRIIA heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-177 of SEQ ID NO: 561 and ends at any one of amino acids 430-458 of SEQ ID NO: 561. In some embodiments, the RGM-A:ActRIIA heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-153 of SEQ ID NO: 565 and ends at any one of amino acids 406-434 of SEQ ID NO: 565. In some embodiments, the RGM-A:ActRIIA heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-169 of SEQ ID NO: 569 and ends at any one of amino acids 422-450 of SEQ ID NO: 569. In some embodiments, a RGM-A:ActRIIA heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 9, 10, and 11. In some embodiments, a RGM-A:ActRIIA heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 21-30 of SEQ ID NO: 9, and ends at any one of amino acids 110-135 of SEQ ID NO: 9. In certain preferred embodiments, RGM-A:ActRIIA heteromultimers are soluble. In some embodiments, a RGM-A:ActRIIA heteromultimer of the disclosure binds to one or more TGF-beta superfamily ligands (e.g., binds to one or more TGF-beta superfamily ligands with a K_{D} of at least 1×10⁻⁷). In some embodiments, a RGM-A:ActRIIA heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a RGM-A:ActRIIA heteromultimer of the disclosure has a different TGF-beta ligand binding and/or inhibition profile (specificity) compared to a corresponding homomultimer (e.g., RGM-A and ActRIIA homomultimers). In some embodiments, a RGM-A:ActRIIA heteromultimer of the disclosure is a heterodimer.

In certain aspects, the disclosure relates to heteromultimers that comprise at least one RGM-A polypeptide, which includes fragments, functional variants, and modified forms thereof, and at least one ActRIIB polypeptide, which includes fragments, functional variants, and modified forms thereof. In some embodiments, the RGM-A:ActRIIB heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 553 or 554. In some embodiments, the RGM-A:ActRIIB heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-177 of SEQ ID NO: 561 and ends at any one of amino acids 430-458 of SEQ ID NO: 561. In some embodiments, the RGM-A:ActRIIB heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-153 of SEQ ID NO: 565 and ends at any one of amino acids 406-434 of SEQ ID NO: 565. In some embodiments, the RGM-A:ActRIIB heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-169 of SEQ ID NO: 569 and ends at any one of amino acids 422-450 of SEQ ID NO: 569. In some embodiments, a RGM-A:ActRIIB heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 1, 2, 3, 4, 5, and 6. In some embodiments, a RGM-A:ActRIIB heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids 20-29 of SEQ ID NO: 1 and ends at a position corresponding to any one of amino acids 109-134 of SEQ ID NO: 1. In some embodiments, a RGM-A:ActRIIB heteromultimer comprises an ActRIIB polypeptide wherein the amino acid position corresponding to L79 of SEQ ID NO: 1 is not an acidic amino acid. In certain preferred embodiments, RGM-A:ActRIIB heteromultimers are soluble. In some embodiments, a RGM-A:ActRIIB heteromultimer of the disclosure binds to one or more TGF-beta superfamily ligands (e.g., binds to one or more TGF-beta superfamily ligands with a K_{D} of at least 1×10⁻⁷). In some embodiments, a RGM-A:ActRIIB heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a RGM-A:ActRIIB heteromultimer of the disclosure has a different TGF-beta ligand binding and/or inhibition profile (specificity) compared to a corresponding homomultimer (e.g., RGM-A and ActRIIB homomultimers). In some embodiments, a RGM-A:ActRIIB heteromultimer of the disclosure is a heterodimer.

In certain aspects, the disclosure relates to heteromultimers that comprise at least one RGM-A polypeptide, which includes fragments, functional variants, and modified forms thereof, and at least one TGFBRII polypeptide, which includes fragments, functional variants, and modified forms thereof. In some embodiments, the RGM-A:TGFBRII heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 553 or 554. In some embodiments, the RGM-A:TGFBRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-177 of SEQ ID NO: 561 and ends at any one of amino acids 430-458 of SEQ ID NO: 561. In some embodiments, the RGM-A:TGFBRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-153 of SEQ ID NO: 565 and ends at any one of amino acids 406-434 of SEQ ID NO: 565. In some embodiments, the RGM-A:TGFBRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-169 of SEQ ID NO: 569 and ends at any one of amino acids 422-450 of SEQ ID NO: 569. In some embodiments, a RGM-A:TGFBRII heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 42, 43, 67, or 68. In some embodiments, a RGM-A:TGFBRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 23-51 of SEQ ID NO: 42 and ends at any one of amino acids 143-166 of SEQ ID NO: 42. In some embodiments, a RGM-A:TGFBRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 23-44 of SEQ ID NO: 67 and ends at any one of amino acids 168-191 of SEQ ID NO: 67. In certain preferred embodiments, RGM-A:TGFBRII heteromultimers are soluble. In some embodiments, a RGM-A:TGFBRII heteromultimer of the disclosure binds to one or more TGF-beta superfamily ligands (e.g., binds to one or more TGF-beta superfamily ligands with a K_{D} of at least 1×10⁻⁷). In some embodiments, a RGM-A:TGFBRII heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a RGM-A:TGFBRII heteromultimer of the disclosure has a different TGF-beta ligand binding and/or inhibition profile (specificity) compared to a corresponding homomultimer (e.g., RGM-A and TGFBRII homomultimers). In some embodiments, a RGM-A:TGFBRII heteromultimer of the disclosure is a heterodimer.

In certain aspects, the disclosure relates to heteromultimers that comprise at least one RGM-A polypeptide, which includes fragments, functional variants, and modified forms thereof, and at least one BMPRII polypeptide, which includes fragments, functional variants, and modified forms thereof. In some embodiments, the RGM-A:BMPRII heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 553 or 554. In some embodiments, the RGM-A:BMPRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-177 of SEQ ID NO: 561 and ends at any one of amino acids 430-458 of SEQ ID NO: 561. In some embodiments, the RGM-A:BMPRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-153 of SEQ ID NO: 565 and ends at any one of amino acids 406-434 of SEQ ID NO: 565. In some embodiments, the RGM-A:BMPRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-169 of SEQ ID NO: 569 and ends at any one of amino acids 422-450 of SEQ ID NO: 569. In some embodiments, a RGM-A:BMPRII heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 46, 47, 71, or 72. In some embodiments, a RGM-A:BMPRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 27-34 of SEQ ID NO: 46 and ends at any one of amino acids 123-150 of SEQ ID NO: 46. In some embodiments, a RGM-A:BMPRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 27-34 of SEQ ID NO: 71 and ends at any one of amino acids 123-150 of SEQ ID NO: 71. In certain preferred embodiments, RGM-A:BMPRII heteromultimers are soluble. In some embodiments, a RGM-A:BMPRII heteromultimer of the disclosure binds to one or more TGF-beta superfamily ligands (e.g., binds to one or more TGF-beta superfamily ligands with a K_{D} of at least 1×10⁻⁷). In some embodiments, a RGM-A:BMPRII heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a RGM-A:BMPRII heteromultimer of the disclosure has a different TGF-beta ligand binding and/or inhibition profile (specificity) compared to a corresponding homomultimer (e.g., RGM-A and BMPRII homomultimers). In some embodiments, a RGM-A:BMPRII heteromultimer of the disclosure is a heterodimer.

In certain aspects, the disclosure relates to heteromultimers that comprise at least one RGM-A polypeptide, which includes fragments, functional variants, and modified forms thereof, and at least one MISRII polypeptide, which includes fragments, functional variants, and modified forms thereof. In some embodiments, the RGM-A:MISRII heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 553 or 554. In some embodiments, the RGM-A:MISRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-177 of SEQ ID NO: 561 and ends at any one of amino acids 430-458 of SEQ ID NO: 561. In some embodiments, the RGM-A:MISRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-153 of SEQ ID NO: 565 and ends at any one of amino acids 406-434 of SEQ ID NO: 565. In some embodiments, the RGM-A:MISRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-169 of SEQ ID NO: 569 and ends at any one of amino acids 422-450 of SEQ ID NO: 569. In some embodiments, a RGM-A:MISRII heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 50, 51, 75, 76, 79, or 80. In some embodiments, a RGM-A:MISRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 17-24 of SEQ ID NO: 50 and ends at any one of amino acids 116-149 of SEQ ID NO: 50. In some embodiments, a RGM-A:MISRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 17-24 of SEQ ID NO: 75 and ends at any one of amino acids 116-149 of SEQ ID NO: 75. In some embodiments, a RGM-A:MISRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 17-24 of SEQ ID NO: 50 and ends at any one of amino acids 116-149 of SEQ ID NO: 79. In certain preferred embodiments, RGM-A:MISRII heteromultimers are soluble. In some embodiments, a RGM-A:MISRII heteromultimer of the disclosure binds to one or more TGF-beta superfamily ligands (e.g., binds to one or more TGF-beta superfamily ligands with a K_{D} of at least 1×10⁻⁷). In some embodiments, a RGM-A:MISRII heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a RGM-A:MISRII heteromultimer of the disclosure has a different TGF-beta ligand binding and/or inhibition profile (specificity) compared to a corresponding homomultimer (e.g., RGM-A and MISRII homomultimers). In some embodiments, a RGM-A:MISRII heteromultimer of the disclosure is a heterodimer.

In certain aspects, the disclosure relates to heteromultimers that comprise at least one RGM-B polypeptide, which includes fragments, functional variants, and modified forms thereof, and at least one ActRIIA polypeptide, which includes fragments, functional variants, and modified forms thereof. In some embodiments, the RGM-B:ActRIIA heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 557 or 558. In some embodiments, the RGM-B:ActRIIA heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-87 of SEQ ID NO: 557 and ends at any one of amino acids 452-478 of SEQ ID NO: 557. In some embodiments, the RGM-B:ActRIIA heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 210-222 of SEQ ID NO: 557 and ends at any one of amino acids 413-452 of SEQ ID NO: 557. In some embodiments, the RGM-B:ActRIIA heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 87-95 of SEQ ID NO: 557 and ends at any one of amino acids 204-209 of SEQ ID NO: 557. In some embodiments, the RGM-B:ActRIIA heteromultimer comprises a RGM-B protein, wherein the RGM-B protein is a dimer comprising a first polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 87-95 of SEQ ID NO: 557 and ends at any one of amino acids 204-209 of SEQ ID NO: 557 and second polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 210-222 of SEQ ID NO: 557 and ends at any one of amino acids 413-452 of SEQ ID NO: 557. In some embodiments, the RGM-B:ActRIIA heteromultimer comprises a single chain ligand trap that comprises a first RGM-B polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 87-95 of SEQ ID NO: 557 and ends at any one of amino acids 204-209 of SEQ ID NO: 557 and second RGM-B polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 210-222 of SEQ ID NO: 557 and ends at any one of amino acids 413-452 of SEQ ID NO: 557. In some embodiments, a RGM-B:ActRIIA heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 9, 10, and 11. In some embodiments, a RGM-B:ActRIIA heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 21-30 of SEQ ID NO: 9, and ends at any one of amino acids 110-135 of SEQ ID NO: 9. In certain preferred embodiments, RGM-B:ActRIIA heteromultimers are soluble. In some embodiments, a RGM-B:ActRIIA heteromultimer of the disclosure binds to one or more TGF-beta superfamily ligands (e.g., binds to one or more TGF-beta superfamily ligands with a K_{D} of at least 1×10⁻⁷). In some embodiments, a RGM-B:ActRIIA heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a RGM-B:ActRIIA heteromultimer of the disclosure has a different TGF-beta ligand binding and/or inhibition profile (specificity) compared to a corresponding homomultimer (e.g., RGM-B and ActRIIA homomultimers). In some embodiments, a RGM-B:ActRIIA heteromultimer of the disclosure is a heterodimer.

In certain aspects, the disclosure relates to heteromultimers that comprise at least one RGM-B polypeptide, which includes fragments, functional variants, and modified forms thereof, and at least one ActRIIB polypeptide, which includes fragments, functional variants, and modified forms thereof. In some embodiments, the RGM-B:ActRIIB heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 557 or 558. In some embodiments, the RGM-B:ActRIIB heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-87 of SEQ ID NO: 557 and ends at any one of amino acids 452-478 of SEQ ID NO: 557. In some embodiments, the RGM-B:ActRIIB heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 210-222 of SEQ ID NO: 557 and ends at any one of amino acids 413-452 of SEQ ID NO: 557. In some embodiments, the RGM-B:ActRIIB heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 87-95 of SEQ ID NO: 557 and ends at any one of amino acids 204-209 of SEQ ID NO: 557. In some embodiments, the RGM-B:ActRIIB heteromultimer comprises a RGM-B protein, wherein the RGM-B protein is a dimer comprising a first polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 87-95 of SEQ ID NO: 557 and ends at any one of amino acids 204-209 of SEQ ID NO: 557 and second polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 210-222 of SEQ ID NO: 557 and ends at any one of amino acids 413-452 of SEQ ID NO: 557. In some embodiments, the RGM-B:ActRIIB heteromultimer comprises a single chain ligand trap that comprises a first RGM-B polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 87-95 of SEQ ID NO: 557 and ends at any one of amino acids 204-209 of SEQ ID NO: 557 and second RGM-B polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 210-222 of SEQ ID NO: 557 and ends at any one of amino acids 413-452 of SEQ ID NO: 557. In some embodiments, a RGM-B:ActRIIB heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 1, 2, 3, 4, 5, and 6. In some embodiments, a RGM-B:ActRIIB heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids 20-29 of SEQ ID NO: 1 and ends at a position corresponding to any one of amino acids 109-134 of SEQ ID NO: 1. In some embodiments, a RGM-B:ActRIIB heteromultimer comprises an ActRIIB polypeptide wherein the amino acid position corresponding to L79 of SEQ ID NO: 1 is not an acidic amino acid. In certain preferred embodiments, RGM-B:ActRIIB heteromultimers are soluble. In some embodiments, a RGM-B:ActRIIB heteromultimer of the disclosure binds to one or more TGF-beta superfamily ligands (e.g., binds to one or more TGF-beta superfamily ligands with a K_{D} of at least 1×10⁻⁷). In some embodiments, a RGM-B:ActRIIB heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a RGM-B:ActRIIB heteromultimer of the disclosure has a different TGF-beta ligand binding and/or inhibition profile (specificity) compared to a corresponding homomultimer (e.g., RGM-B and ActRIIB homomultimers). In some embodiments, a RGM-B:ActRIIB heteromultimer of the disclosure is a heterodimer.

In certain aspects, the disclosure relates to heteromultimers that comprise at least one RGM-B polypeptide, which includes fragments, functional variants, and modified forms thereof, and at least one TGFBRII polypeptide, which includes fragments, functional variants, and modified forms thereof. In some embodiments, the RGM-B:TGFBRII heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 557 or 558. In some embodiments, the RGM-B:TGFBRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-87 of SEQ ID NO: 557 and ends at any one of amino acids 452-478 of SEQ ID NO: 557. In some embodiments, the RGM-B:TGFBRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 210-222 of SEQ ID NO: 557 and ends at any one of amino acids 413-452 of SEQ ID NO: 557. In some embodiments, the RGM-B:TGFBRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 87-95 of SEQ ID NO: 557 and ends at any one of amino acids 204-209 of SEQ ID NO: 557. In some embodiments, the RGM-B:TGFBRII heteromultimer comprises a RGM-B protein, wherein the RGM-B protein is a dimer comprising a first polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 87-95 of SEQ ID NO: 557 and ends at any one of amino acids 204-209 of SEQ ID NO: 557 and second polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 210-222 of SEQ ID NO: 557 and ends at any one of amino acids 413-452 of SEQ ID NO: 557. In some embodiments, the RGM-B:TGFBRII heteromultimer comprises a single chain ligand trap that comprises a first RGM-B polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 87-95 of SEQ ID NO: 557 and ends at any one of amino acids 204-209 of SEQ ID NO: 557 and second RGM-B polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 210-222 of SEQ ID NO: 557 and ends at any one of amino acids 413-452 of SEQ ID NO: 557. In some embodiments, a RGM-B:TGFBRII heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 42, 43, 67, or 68. In some embodiments, a RGM-B:TGFBRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 23-51 of SEQ ID NO: 42 and ends at any one of amino acids 143-166 of SEQ ID NO: 42. In some embodiments, a RGM-B:TGFBRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 23-44 of SEQ ID NO: 67 and ends at any one of amino acids 168-191 of SEQ ID NO: 67. In certain preferred embodiments, RGM-B:TGFBRII heteromultimers are soluble. In some embodiments, a RGM-B:TGFBRII heteromultimer of the disclosure binds to one or more TGF-beta superfamily ligands (e.g., binds to one or more TGF-beta superfamily ligands with a K_{D} of at least 1×10⁻⁷). In some embodiments, a RGM-B:TGFBRII heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a RGM-B:TGFBRII heteromultimer of the disclosure has a different TGF-beta ligand binding and/or inhibition profile (specificity) compared to a corresponding homomultimer (e.g., RGM-B and TGFBRII homomultimers). In some embodiments, a RGM-B:TGFBRII heteromultimer of the disclosure is a heterodimer.

In certain aspects, the disclosure relates to heteromultimers that comprise at least one RGM-B polypeptide, which includes fragments, functional variants, and modified forms thereof, and at least one BMPRII polypeptide, which includes fragments, functional variants, and modified forms thereof. In some embodiments, the RGM-B:BMPRII heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 557 or 558. In some embodiments, the RGM-B:BMPRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-87 of SEQ ID NO: 557 and ends at any one of amino acids 452-478 of SEQ ID NO: 557. In some embodiments, the RGM-B:BMPRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 210-222 of SEQ ID NO: 557 and ends at any one of amino acids 413-452 of SEQ ID NO: 557. In some embodiments, the RGM-B:BMPRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 87-95 of SEQ ID NO: 557 and ends at any one of amino acids 204-209 of SEQ ID NO: 557. In some embodiments, the RGM-B:BMPRII heteromultimer comprises a RGM-B protein, wherein the RGM-B protein is a dimer comprising a first polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 87-95 of SEQ ID NO: 557 and ends at any one of amino acids 204-209 of SEQ ID NO: 557 and second polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 210-222 of SEQ ID NO: 557 and ends at any one of amino acids 413-452 of SEQ ID NO: 557. In some embodiments, the RGM-B:BMPRII heteromultimer comprises a single chain ligand trap that comprises a first RGM-B polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 87-95 of SEQ ID NO: 557 and ends at any one of amino acids 204-209 of SEQ ID NO: 557 and second RGM-B polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 210-222 of SEQ ID NO: 557 and ends at any one of amino acids 413-452 of SEQ ID NO: 557. In some embodiments, a RGM-B:BMPRII heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 46, 47, 71, or 72. In some embodiments, a RGM-B:BMPRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 27-34 of SEQ ID NO: 46 and ends at any one of amino acids 123-150 of SEQ ID NO: 46. In some embodiments, a RGM-B:BMPRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 27-34 of SEQ ID NO: 71 and ends at any one of amino acids 123-150 of SEQ ID NO: 71. In certain preferred embodiments, RGM-B:BMPRII heteromultimers are soluble. In some embodiments, a RGM-B:BMPRII heteromultimer of the disclosure binds to one or more TGF-beta superfamily ligands (e.g., binds to one or more TGF-beta superfamily ligands with a K_{D} of at least 1×10⁻⁷). In some embodiments, a RGM-B:BMPRII heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a RGM-B:BMPRII heteromultimer of the disclosure has a different TGF-beta ligand binding and/or inhibition profile (specificity) compared to a corresponding homomultimer (e.g., RGM-B and BMPRII homomultimers). In some embodiments, a RGM-B:BMPRII heteromultimer of the disclosure is a heterodimer.

In certain aspects, the disclosure relates to heteromultimers that comprise at least one RGM-B polypeptide, which includes fragments, functional variants, and modified forms thereof, and at least one MISRII polypeptide, which includes fragments, functional variants, and modified forms thereof. In some embodiments, the RGM-B:MISRII heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 557 or 558. In some embodiments, the RGM-B:MISRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-87 of SEQ ID NO: 557 and ends at any one of amino acids 452-478 of SEQ ID NO: 557. In some embodiments, the RGM-B:MISRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 210-222 of SEQ ID NO: 557 and ends at any one of amino acids 413-452 of SEQ ID NO: 557. In some embodiments, the RGM-B:MISRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 87-95 of SEQ ID NO: 557 and ends at any one of amino acids 204-209 of SEQ ID NO: 557. In some embodiments, the RGM-B:MISRII heteromultimer comprises a RGM-B protein, wherein the RGM-B protein is a dimer comprising a first polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 87-95 of SEQ ID NO: 557 and ends at any one of amino acids 204-209 of SEQ ID NO: 557 and second polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 210-222 of SEQ ID NO: 557 and ends at any one of amino acids 413-452 of SEQ ID NO: 557. In some embodiments, the RGM-B:MISRII heteromultimer comprises a single chain ligand trap that comprises a first RGM-B polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 87-95 of SEQ ID NO: 557 and ends at any one of amino acids 204-209 of SEQ ID NO: 557 and second RGM-B polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 210-222 of SEQ ID NO: 557 and ends at any one of amino acids 413-452 of SEQ ID NO: 557. In some embodiments, a RGM-B:MISRII heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 50, 51, 75, 76, 79, or 80. In some embodiments, a RGM-B:MISRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 17-24 of SEQ ID NO: 50 and ends at any one of amino acids 116-149 of SEQ ID NO: 50. In some embodiments, a RGM-B:MISRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 17-24 of SEQ ID NO: 75 and ends at any one of amino acids 116-149 of SEQ ID NO: 75. In some embodiments, a RGM-B:MISRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 17-24 of SEQ ID NO: 50 and ends at any one of amino acids 116-149 of SEQ ID NO: 79. In certain preferred embodiments, RGM-B:MISRII heteromultimers are soluble. In some embodiments, a RGM-B:MISRII heteromultimer of the disclosure binds to one or more TGF-beta superfamily ligands (e.g., binds to one or more TGF-beta superfamily ligands with a K_{D} of at least 1×10⁻⁷). In some embodiments, a RGM-B:MISRII heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a RGM-B:MISRII heteromultimer of the disclosure has a different TGF-beta ligand binding and/or inhibition profile (specificity) compared to a corresponding homomultimer (e.g., RGM-B and MISRII homomultimers). In some embodiments, a RGM-B:MISRII heteromultimer of the disclosure is a heterodimer.

In certain aspects, the disclosure relates to heteromultimers that comprise at least one hemojuvelin polypeptide, which includes fragments, functional variants, and modified forms thereof, and at least one ActRIIA polypeptide, which includes fragments, functional variants, and modified forms thereof. In some embodiments, the hemojuvelin:ActRIIA heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 573, 574, 577, 578, 581, or 582. In some embodiments, the hemojuvelin:ActRIIA heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-36 of SEQ ID NO: 573 and ends at any one of amino acids 400-426 of SEQ ID NO: 573. In some embodiments, the hemojuvelin:ActRIIA heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 36-42 of SEQ ID NO: 573 and ends at any one of amino acids 167-172 of SEQ ID NO: 573. In some embodiments, the hemojuvelin:ActRIIA heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 173-185 of SEQ ID NO: 573 and ends at any one of amino acids 361-400 of SEQ ID NO: 573. In some embodiments, the hemojuvelin:ActRIIA heteromultimer comprises a hemojuvelin protein that is a dimer comprising a first polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 36-42 of SEQ ID NO: 573, and ends at any one of amino acids 167-172 of SEQ ID NO: 573 and second polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 173-185 of SEQ ID NO: 573 and ends at any one of amino acids 361-400 of SEQ ID NO: 573. In some embodiments, the hemojuvelin:ActRIIA heteromultimer comprises a single chain ligand trap that comprises a first hemojuvelin polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 36-42 of SEQ ID NO: 573 and ends at any one of amino acids 167-172 of SEQ ID NO: 573 and second hemojuvelin polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 173-185 of SEQ ID NO: 573 and ends at any one of amino acids 361-400 of SEQ ID NO: 573. In some embodiments, the hemojuvelin:ActRIIA heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-6 of SEQ ID NO: 577 and ends at any one of amino acids 287-313 of SEQ ID NO: 577. In some embodiments, the hemojuvelin:ActRIIA heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-6 of SEQ ID NO: 577 and ends at any one of amino acids 54-59 of SEQ ID NO: 577. In some embodiments, the hemojuvelin:ActRIIA heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 60-72 of SEQ ID NO: 577 and ends at any one of amino acids 248-287 of SEQ ID NO: 577. In some embodiments, the hemojuvelin:ActRIIA heteromultimer comprises a hemojuvelin protein, wherein the hemojuvelin protein is a dimer comprising a first polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 1-6 of SEQ ID NO: 577 and ends at any one of amino acids 54-59 of SEQ ID NO: 577, and second polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 60-72 of SEQ ID NO: 577 and ends at any one of amino acids 248-287 of SEQ ID NO: 577. In some embodiments, the hemojuvelin:ActRIIA heteromultimer comprises a single chain ligand trap that comprises a first hemojuvelin polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 1-6 of SEQ ID NO: 577 and ends at any one of amino acids 54-59 of SEQ ID NO: 577, and second hemojuvelin polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 60-72 of SEQ ID NO: 577, and ends at any one of amino acids 248-287 of SEQ ID NO: 577. In some embodiments, the hemojuvelin:ActRIIA heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-4 of SEQ ID NO: 581 and ends at any one of amino acids 135-200 of SEQ ID NO: 581. In some embodiments, a hemojuvelin:ActRIIA heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 9, 10, and 11. In some embodiments, a hemojuvelin:ActRIIA heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 21-30 of SEQ ID NO: 9, and ends at any one of amino acids 110-135 of SEQ ID NO: 9. In certain preferred embodiments, hemojuvelin:ActRIIA heteromultimers are soluble. In some embodiments, a hemojuvelin:ActRIIA heteromultimer of the disclosure binds to one or more TGF-beta superfamily ligands (e.g., binds to one or more TGF-beta superfamily ligands with a Kr) of at least 1×10⁻⁷). In some embodiments, a hemojuvelin:ActRIIA heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a hemojuvelin:ActRIIA heteromultimer of the disclosure has a different TGF-beta ligand binding and/or inhibition profile (specificity) compared to a corresponding homomultimer (e.g., hemojuvelin and ActRIIA homomultimers). In some embodiments, a hemojuvelin:ActRIIA heteromultimer of the disclosure is a heterodimer.

In certain aspects, the disclosure relates to heteromultimers that comprise at least one hemojuvelin polypeptide, which includes fragments, functional variants, and modified forms thereof, and at least one ActRIIB polypeptide, which includes fragments, functional variants, and modified forms thereof. In some embodiments, the hemojuvelin:ActRIIB heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 573, 574, 577, 578, 581, or 582. In some embodiments, the hemojuvelin:ActRIIB heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-36 of SEQ ID NO: 573 and ends at any one of amino acids 400-426 of SEQ ID NO: 573. In some embodiments, the hemojuvelin:ActRIIB heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 36-42 of SEQ ID NO: 573 and ends at any one of amino acids 167-172 of SEQ ID NO: 573. In some embodiments, the hemojuvelin:ActRIIB heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 173-185 of SEQ ID NO: 573 and ends at any one of amino acids 361-400 of SEQ ID NO: 573. In some embodiments, the hemojuvelin:ActRIIB heteromultimer comprises a hemojuvelin protein that is a dimer comprising a first polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 36-42 of SEQ ID NO: 573, and ends at any one of amino acids 167-172 of SEQ ID NO: 573 and second polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 173-185 of SEQ ID NO: 573 and ends at any one of amino acids 361-400 of SEQ ID NO: 573. In some embodiments, the hemojuvelin:ActRIIB heteromultimer comprises a single chain ligand trap that comprises a first hemojuvelin polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 36-42 of SEQ ID NO: 573 and ends at any one of amino acids 167-172 of SEQ ID NO: 573 and second hemojuvelin polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 173-185 of SEQ ID NO: 573 and ends at any one of amino acids 361-400 of SEQ ID NO: 573. In some embodiments, the hemojuvelin:ActRIIB heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-6 of SEQ ID NO: 577 and ends at any one of amino acids 287-313 of SEQ ID NO: 577. In some embodiments, the hemojuvelin:ActRIIB heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-6 of SEQ ID NO: 577 and ends at any one of amino acids 54-59 of SEQ ID NO: 577. In some embodiments, the hemojuvelin:ActRIIB heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 60-72 of SEQ ID NO: 577 and ends at any one of amino acids 248-287 of SEQ ID NO: 577. In some embodiments, the hemojuvelin:ActRIIB heteromultimer comprises a hemojuvelin protein, wherein the hemojuvelin protein is a dimer comprising a first polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 1-6 of SEQ ID NO: 577 and ends at any one of amino acids 54-59 of SEQ ID NO: 577, and second polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 60-72 of SEQ ID NO: 577 and ends at any one of amino acids 248-287 of SEQ ID NO: 577. In some embodiments, the hemojuvelin:ActRIIB heteromultimer comprises a single chain ligand trap that comprises a first hemojuvelin polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 1-6 of SEQ ID NO: 577 and ends at any one of amino acids 54-59 of SEQ ID NO: 577, and second hemojuvelin polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 60-72 of SEQ ID NO: 577, and ends at any one of amino acids 248-287 of SEQ ID NO: 577. In some embodiments, the hemojuvelin:ActRIIB heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-4 of SEQ ID NO: 581 and ends at any one of amino acids 135-200 of SEQ ID NO: 581. In some embodiments, a hemojuvelin:ActRIIB heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 1, 2, 3, 4, 5, and 6. In some embodiments, a hemojuvelin:ActRIIB heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids 20-29 of SEQ ID NO: 1 and ends at a position corresponding to any one of amino acids 109-134 of SEQ ID NO: 1. In some embodiments, a hemojuvelin:ActRIIB heteromultimer comprises an ActRIIB polypeptide wherein the amino acid position corresponding to L79 of SEQ ID NO: 1 is not an acidic amino acid. In certain preferred embodiments, hemojuvelin:ActRIIB heteromultimers are soluble. In some embodiments, a hemojuvelin:ActRIIB heteromultimer of the disclosure binds to one or more TGF-beta superfamily ligands (e.g., binds to one or more TGF-beta superfamily ligands with a K_{D} of at least 1×10⁻⁷). In some embodiments, a hemojuvelin:ActRIIB heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a hemojuvelin:ActRIIB heteromultimer of the disclosure has a different TGF-beta ligand binding and/or inhibition profile (specificity) compared to a corresponding homomultimer (e.g., hemojuvelin and ActRIIB homomultimers). In some embodiments, a hemojuvelin:ActRIIB heteromultimer of the disclosure is a heterodimer.

In certain aspects, the disclosure relates to heteromultimers that comprise at least one hemojuvelin polypeptide, which includes fragments, functional variants, and modified forms thereof, and at least one TGFBRII polypeptide, which includes fragments, functional variants, and modified forms thereof. In some embodiments, the hemojuvelin:TGFBRII heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 573, 574, 577, 578, 581, or 582. In some embodiments, the hemojuvelin:TGFBRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-36 of SEQ ID NO: 573 and ends at any one of amino acids 400-426 of SEQ ID NO: 573. In some embodiments, the hemojuvelin:TGFBRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 36-42 of SEQ ID NO: 573 and ends at any one of amino acids 167-172 of SEQ ID NO: 573. In some embodiments, the hemojuvelin:TGFBRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 173-185 of SEQ ID NO: 573 and ends at any one of amino acids 361-400 of SEQ ID NO: 573. In some embodiments, the hemojuvelin:TGFBRII heteromultimer comprises a hemojuvelin protein that is a dimer comprising a first polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 36-42 of SEQ ID NO: 573, and ends at any one of amino acids 167-172 of SEQ ID NO: 573 and second polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 173-185 of SEQ ID NO: 573 and ends at any one of amino acids 361-400 of SEQ ID NO: 573. In some embodiments, the hemojuvelin:TGFBRII heteromultimer comprises a single chain ligand trap that comprises a first hemojuvelin polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 36-42 of SEQ ID NO: 573 and ends at any one of amino acids 167-172 of SEQ ID NO: 573 and second hemojuvelin polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 173-185 of SEQ ID NO: 573 and ends at any one of amino acids 361-400 of SEQ ID NO: 573. In some embodiments, the hemojuvelin:TGFBRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-6 of SEQ ID NO: 577 and ends at any one of amino acids 287-313 of SEQ ID NO: 577. In some embodiments, the hemojuvelin:TGFBRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-6 of SEQ ID NO: 577 and ends at any one of amino acids 54-59 of SEQ ID NO: 577. In some embodiments, the hemojuvelin:TGFBRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 60-72 of SEQ ID NO: 577 and ends at any one of amino acids 248-287 of SEQ ID NO: 577. In some embodiments, the hemojuvelin:TGFBRII heteromultimer comprises a hemojuvelin protein, wherein the hemojuvelin protein is a dimer comprising a first polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 1-6 of SEQ ID NO: 577 and ends at any one of amino acids 54-59 of SEQ ID NO: 577, and second polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 60-72 of SEQ ID NO: 577 and ends at any one of amino acids 248-287 of SEQ ID NO: 577. In some embodiments, the hemojuvelin:TGFBRII heteromultimer comprises a single chain ligand trap that comprises a first hemojuvelin polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 1-6 of SEQ ID NO: 577 and ends at any one of amino acids 54-59 of SEQ ID NO: 577, and second hemojuvelin polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 60-72 of SEQ ID NO: 577, and ends at any one of amino acids 248-287 of SEQ ID NO: 577. In some embodiments, the hemojuvelin:TGFBRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-4 of SEQ ID NO: 581 and ends at any one of amino acids 135-200 of SEQ ID NO: 581. In some embodiments, a hemojuvelin:TGFBRII heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 42, 43, 67, or 68. In some embodiments, a hemojuvelin:TGFBRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 23-51 of SEQ ID NO: 42 and ends at any one of amino acids 143-166 of SEQ ID NO: 42. In some embodiments, a hemojuvelin:TGFBRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 23-44 of SEQ ID NO: 67 and ends at any one of amino acids 168-191 of SEQ ID NO: 67. In certain preferred embodiments, hemojuvelin:TGFBRII heteromultimers are soluble. In some embodiments, a hemojuvelin:TGFBRII heteromultimer of the disclosure binds to one or more TGF-beta superfamily ligands (e.g., binds to one or more TGF-beta superfamily ligands with a K_{D} of at least 1×10⁻⁷). In some embodiments, a hemojuvelin:TGFBRII heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a hemojuvelin:TGFBRII heteromultimer of the disclosure has a different TGF-beta ligand binding and/or inhibition profile (specificity) compared to a corresponding homomultimer (e.g., hemojuvelin and TGFBRII homomultimers). In some embodiments, a hemojuvelin:TGFBRII heteromultimer of the disclosure is a heterodimer.

In certain aspects, the disclosure relates to heteromultimers that comprise at least one hemojuvelin polypeptide, which includes fragments, functional variants, and modified forms thereof, and at least one BMPRII polypeptide, which includes fragments, functional variants, and modified forms thereof. In some embodiments, the hemojuvelin:BMPRII heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 573, 574, 577, 578, 581, or 582. In some embodiments, the hemojuvelin:BMPRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-36 of SEQ ID NO: 573 and ends at any one of amino acids 400-426 of SEQ ID NO: 573. In some embodiments, the hemojuvelin:BMPRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 36-42 of SEQ ID NO: 573 and ends at any one of amino acids 167-172 of SEQ ID NO: 573. In some embodiments, the hemojuvelin:BMPRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 173-185 of SEQ ID NO: 573 and ends at any one of amino acids 361-400 of SEQ ID NO: 573. In some embodiments, the hemojuvelin:BMPRII heteromultimer comprises a hemojuvelin protein that is a dimer comprising a first polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 36-42 of SEQ ID NO: 573, and ends at any one of amino acids 167-172 of SEQ ID NO: 573 and second polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 173-185 of SEQ ID NO: 573 and ends at any one of amino acids 361-400 of SEQ ID NO: 573. In some embodiments, the hemojuvelin:BMPRII heteromultimer comprises a single chain ligand trap that comprises a first hemojuvelin polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 36-42 of SEQ ID NO: 573 and ends at any one of amino acids 167-172 of SEQ ID NO: 573 and second hemojuvelin polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 173-185 of SEQ ID NO: 573 and ends at any one of amino acids 361-400 of SEQ ID NO: 573. In some embodiments, the hemojuvelin:BMPRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-6 of SEQ ID NO: 577 and ends at any one of amino acids 287-313 of SEQ ID NO: 577. In some embodiments, the hemojuvelin:BMPRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-6 of SEQ ID NO: 577 and ends at any one of amino acids 54-59 of SEQ ID NO: 577. In some embodiments, the hemojuvelin:BMPRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 60-72 of SEQ ID NO: 577 and ends at any one of amino acids 248-287 of SEQ ID NO: 577. In some embodiments, the hemojuvelin:BMPRII heteromultimer comprises a hemojuvelin protein, wherein the hemojuvelin protein is a dimer comprising a first polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 1-6 of SEQ ID NO: 577 and ends at any one of amino acids 54-59 of SEQ ID NO: 577, and second polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 60-72 of SEQ ID NO: 577 and ends at any one of amino acids 248-287 of SEQ ID NO: 577. In some embodiments, the hemojuvelin:BMPRII heteromultimer comprises a single chain ligand trap that comprises a first hemojuvelin polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 1-6 of SEQ ID NO: 577 and ends at any one of amino acids 54-59 of SEQ ID NO: 577, and second hemojuvelin polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 60-72 of SEQ ID NO: 577, and ends at any one of amino acids 248-287 of SEQ ID NO: 577. In some embodiments, the hemojuvelin:BMPRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-4 of SEQ ID NO: 581 and ends at any one of amino acids 135-200 of SEQ ID NO: 581. In some embodiments, a hemojuvelin:BMPRII heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 46, 47, 71, or 72. In some embodiments, a hemojuvelin:BMPRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 27-34 of SEQ ID NO: 46 and ends at any one of amino acids 123-150 of SEQ ID NO: 46. In some embodiments, a hemojuvelin:BMPRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 27-34 of SEQ ID NO: 71 and ends at any one of amino acids 123-150 of SEQ ID NO: 71. In certain preferred embodiments, hemojuvelin:BMPRII heteromultimers are soluble. In some embodiments, a hemojuvelin:BMPRII heteromultimer of the disclosure binds to one or more TGF-beta superfamily ligands (e.g., binds to one or more TGF-beta superfamily ligands with a K_{D} of at least 1×10⁻⁷). In some embodiments, a hemojuvelin:BMPRII heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a hemojuvelin:BMPRII heteromultimer of the disclosure has a different TGF-beta ligand binding and/or inhibition profile (specificity) compared to a corresponding homomultimer (e.g., hemojuvelin and BMPRII homomultimers). In some embodiments, a hemojuvelin:BMPRII heteromultimer of the disclosure is a heterodimer.

In certain aspects, the disclosure relates to heteromultimers that comprise at least one hemojuvelin polypeptide, which includes fragments, functional variants, and modified forms thereof, and at least one MISRII polypeptide, which includes fragments, functional variants, and modified forms thereof. In some embodiments, the hemojuvelin:MISRII heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 573, 574, 577, 578, 581, or 582. In some embodiments, the hemojuvelin:MISRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-36 of SEQ ID NO: 573 and ends at any one of amino acids 400-426 of SEQ ID NO: 573. In some embodiments, the hemojuvelin:MISRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 36-42 of SEQ ID NO: 573 and ends at any one of amino acids 167-172 of SEQ ID NO: 573. In some embodiments, the hemojuvelin:MISRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 173-185 of SEQ ID NO: 573 and ends at any one of amino acids 361-400 of SEQ ID NO: 573. In some embodiments, the hemojuvelin:MISRII heteromultimer comprises a hemojuvelin protein that is a dimer comprising a first polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 36-42 of SEQ ID NO: 573, and ends at any one of amino acids 167-172 of SEQ ID NO: 573 and second polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 173-185 of SEQ ID NO: 573 and ends at any one of amino acids 361-400 of SEQ ID NO: 573. In some embodiments, the hemojuvelin:MISRII heteromultimer comprises a single chain ligand trap that comprises a first hemojuvelin polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 36-42 of SEQ ID NO: 573 and ends at any one of amino acids 167-172 of SEQ ID NO: 573 and second hemojuvelin polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 173-185 of SEQ ID NO: 573 and ends at any one of amino acids 361-400 of SEQ ID NO: 573. In some embodiments, the hemojuvelin:MISRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-6 of SEQ ID NO: 577 and ends at any one of amino acids 287-313 of SEQ ID NO: 577. In some embodiments, the hemojuvelin:MISRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-6 of SEQ ID NO: 577 and ends at any one of amino acids 54-59 of SEQ ID NO: 577. In some embodiments, the hemojuvelin:MISRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 60-72 of SEQ ID NO: 577 and ends at any one of amino acids 248-287 of SEQ ID NO: 577. In some embodiments, the hemojuvelin:MISRII heteromultimer comprises a hemojuvelin protein, wherein the hemojuvelin protein is a dimer comprising a first polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 1-6 of SEQ ID NO: 577 and ends at any one of amino acids 54-59 of SEQ ID NO: 577, and second polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 60-72 of SEQ ID NO: 577 and ends at any one of amino acids 248-287 of SEQ ID NO: 577. In some embodiments, the hemojuvelin:MISRII heteromultimer comprises a single chain ligand trap that comprises a first hemojuvelin polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 1-6 of SEQ ID NO: 577 and ends at any one of amino acids 54-59 of SEQ ID NO: 577, and second hemojuvelin polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 60-72 of SEQ ID NO: 577, and ends at any one of amino acids 248-287 of SEQ ID NO: 577. In some embodiments, the hemojuvelin:MISRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-4 of SEQ ID NO: 581 and ends at any one of amino acids 135-200 of SEQ ID NO: 581. In some embodiments, a hemojuvelin:MISRII heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 50, 51, 75, 76, 79, or 80. In some embodiments, a hemojuvelin:MISRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 17-24 of SEQ ID NO: 50 and ends at any one of amino acids 116-149 of SEQ ID NO: 50. In some embodiments, a hemojuvelin:MISRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 17-24 of SEQ ID NO: 75 and ends at any one of amino acids 116-149 of SEQ ID NO: 75. In some embodiments, a hemojuvelin:MISRII heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 17-24 of SEQ ID NO: 50 and ends at any one of amino acids 116-149 of SEQ ID NO: 79. In certain preferred embodiments, hemojuvelin:MISRII heteromultimers are soluble. In some embodiments, a hemojuvelin:MISRII heteromultimer of the disclosure binds to one or more TGF-beta superfamily ligands (e.g., binds to one or more TGF-beta superfamily ligands with a K_{D} of at least 1×10⁻⁷). In some embodiments, a hemojuvelin:MISRII heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a hemojuvelin:MISRII heteromultimer of the disclosure has a different TGF-beta ligand binding and/or inhibition profile (specificity) compared to a corresponding homomultimer (e.g., hemojuvelin and MISRII homomultimers). In some embodiments, a hemojuvelin:MISRII heteromultimer of the disclosure is a heterodimer.

In certain aspects, the disclosure relates to heteromultimers that comprise at least one endoglin polypeptide, which includes fragments, functional variants, and modified forms thereof, and at least one betaglycan polypeptide, which includes fragments, functional variants, and modified forms thereof. In some embodiments, the endoglin:betaglycan heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 501, 502, 505, 506, 509, 510, 593, or 594. In some embodiments, the endoglin:betaglycan heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-30 of SEQ ID NO: 501 and ends at any one of amino acids 330-346 of SEQ ID NO: 501. In some embodiments, the endoglin:betaglycan heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-30 of SEQ ID NO: 505 and ends at any one of amino acids 330-346 of SEQ ID NO: 505. In some embodiments, the endoglin:betaglycan heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-25 of SEQ ID NO: 509, and ends at any one of amino acids 148-164 of SEQ ID NO: 509. In some embodiments, the endoglin:betaglycan heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 585, 586, 589, or 590. In some embodiments, the endoglin:betaglycan heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 21-28 of SEQ ID NO: 585 and ends at any one of amino acids 381-787 of SEQ ID NO: 585. In some embodiments, the endoglin:betaglycan heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 21-28 of SEQ ID NO: 589 and ends at any one of amino acids 380-786 of SEQ ID NO: 589. In certain preferred embodiments, endoglin:betaglycan heteromultimers are soluble. In some embodiments, a endoglin:betaglycan heteromultimer of the disclosure binds to one or more TGF-beta superfamily ligands (e.g., binds to one or more TGF-beta superfamily ligands with a K_{D} of at least 1×10⁻⁷). In some embodiments, a endoglin:betaglycan heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a endoglin:betaglycan heteromultimer of the disclosure has a different TGF-beta ligand binding and/or inhibition profile (specificity) compared to a corresponding homomultimer (e.g., endoglin and betaglycan homomultimers). In some embodiments, a endoglin:betaglycan heteromultimer of the disclosure is a heterodimer.

In certain aspects, the disclosure relates to heteromultimers that comprise at least one endoglin polypeptide, which includes fragments, functional variants, and modified forms thereof, and at least one Cripto-1 polypeptide, which includes fragments, functional variants, and modified forms thereof. In some embodiments, the endoglin:Cripto-1 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 501, 502, 505, 506, 509, 510, 593, or 594. In some embodiments, the endoglin:Cripto-1 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-30 of SEQ ID NO: 501 and ends at any one of amino acids 330-346 of SEQ ID NO: 501. In some embodiments, the endoglin:Cripto-1 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-30 of SEQ ID NO: 505 and ends at any one of amino acids 330-346 of SEQ ID NO: 505. In some embodiments, the endoglin:Cripto-1 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-25 of SEQ ID NO: 509, and ends at any one of amino acids 148-164 of SEQ ID NO: 509. In some embodiments, the endoglin:Cripto-1 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 513, 514, 517, or 518. In some embodiments, the endoglin:Cripto-1 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 31-82 of SEQ ID NO: 513 and ends at any one of amino acids 172-188 of SEQ ID NO: 513. In some embodiments, the endoglin:Cripto-1 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 15-66 of SEQ ID NO: 517, and ends at any one of amino acids 156-172 of SEQ ID NO: 517. In certain preferred embodiments, endoglin:Cripto-1 heteromultimers are soluble. In some embodiments, a endoglin:Cripto-1 heteromultimer of the disclosure binds to one or more TGF-beta superfamily ligands (e.g., binds to one or more TGF-beta superfamily ligands with a K_{D} of at least 1×10⁻⁷). In some embodiments, a endoglin:Cripto-1 heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a endoglin:Cripto-1 heteromultimer of the disclosure has a different TGF-beta ligand binding and/or inhibition profile (specificity) compared to a corresponding homomultimer (e.g., endoglin and Cripto-1 homomultimers). In some embodiments, a endoglin:Cripto-1 heteromultimer of the disclosure is a heterodimer.

In certain aspects, the disclosure relates to heteromultimers that comprise at least one endoglin polypeptide, which includes fragments, functional variants, and modified forms thereof, and at least one Cryptic protein polypeptide, which includes fragments, functional variants, and modified forms thereof. In some embodiments, the endoglin:Cryptic protein heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 501, 502, 505, 506, 509, 510, 593, or 594. In some embodiments, the endoglin:Cryptic protein heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-30 of SEQ ID NO: 501 and ends at any one of amino acids 330-346 of SEQ ID NO: 501. In some embodiments, the endoglin:Cryptic protein heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-30 of SEQ ID NO: 505 and ends at any one of amino acids 330-346 of SEQ ID NO: 505. In some embodiments, the endoglin:Cryptic protein heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-25 of SEQ ID NO: 509, and ends at any one of amino acids 148-164 of SEQ ID NO: 509. In some embodiments, the endoglin:Cryptic protein heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 521, 522, 525, 526, 529, or 530. In some embodiments, the endoglin:Cryptic protein heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-90 of SEQ ID NO: 521 and ends at any one of amino acids 157-233 of SEQ ID NO: 521. In some embodiments, the endoglin:Cryptic protein heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-30 of SEQ ID NO: 525 and ends at any one of amino acids 82-191 of SEQ ID NO: 525. In some embodiments, the endoglin:Cryptic protein heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-30 of SEQ ID NO: 529, and ends at any one of amino acids 82-148 of SEQ ID NO: 529. In certain preferred embodiments, endoglin:Cryptic protein heteromultimers are soluble. In some embodiments, a endoglin:Cryptic protein heteromultimer of the disclosure binds to one or more TGF-beta superfamily ligands (e.g., binds to one or more TGF-beta superfamily ligands with a K_{D} of at least 1×10⁻⁷). In some embodiments, a endoglin:Cryptic protein heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a endoglin:Cryptic protein heteromultimer of the disclosure has a different TGF-beta ligand binding and/or inhibition profile (specificity) compared to a corresponding homomultimer (e.g., endoglin and Cryptic protein homomultimers). In some embodiments, a endoglin:Cryptic protein heteromultimer of the disclosure is a heterodimer.

In certain aspects, the disclosure relates to heteromultimers that comprise at least one endoglin polypeptide, which includes fragments, functional variants, and modified forms thereof, and at least one Cryptic family protein 1B polypeptide, which includes fragments, functional variants, and modified forms thereof. In some embodiments, the endoglin:Cryptic family protein 1B heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 501, 502, 505, 506, 509, 510, 593, or 594. In some embodiments, the endoglin:Cryptic family protein 1B heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-30 of SEQ ID NO: 501 and ends at any one of amino acids 330-346 of SEQ ID NO: 501. In some embodiments, the endoglin:Cryptic family protein 1B heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-30 of SEQ ID NO: 505 and ends at any one of amino acids 330-346 of SEQ ID NO: 505. In some embodiments, the endoglin:Cryptic family protein 1B heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-25 of SEQ ID NO: 509, and ends at any one of amino acids 148-164 of SEQ ID NO: 509. In some embodiments, the endoglin:Cryptic family protein 1B heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 533 or 534. In some embodiments, the endoglin:Cryptic family protein 1B heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-30 of SEQ ID NO: 533 and ends at any one of amino acids 82-223 of SEQ ID NO: 533. In certain preferred embodiments, endoglin:Cryptic family protein 1B heteromultimers are soluble. In some embodiments, a endoglin:Cryptic family protein 1B heteromultimer of the disclosure binds to one or more TGF-beta superfamily ligands (e.g., binds to one or more TGF-beta superfamily ligands with a K_{D} of at least 1×10⁻⁷). In some embodiments, a endoglin:Cryptic family protein 1B heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a endoglin:Cryptic family protein 1B heteromultimer of the disclosure has a different TGF-beta ligand binding and/or inhibition profile (specificity) compared to a corresponding homomultimer (e.g., endoglin and Cryptic family protein 1B homomultimers). In some embodiments, a endoglin:Cryptic family protein 1B heteromultimer of the disclosure is a heterodimer.

In certain aspects, the disclosure relates to heteromultimers that comprise at least one endoglin polypeptide, which includes fragments, functional variants, and modified forms thereof, and at least one Crim1 polypeptide, which includes fragments, functional variants, and modified forms thereof. In some embodiments, the endoglin:Crim1 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 501, 502, 505, 506, 509, 510, 593, or 594. In some embodiments, the endoglin:Crim1 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-30 of SEQ ID NO: 501 and ends at any one of amino acids 330-346 of SEQ ID NO: 501. In some embodiments, the endoglin:Crim1 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-30 of SEQ ID NO: 505 and ends at any one of amino acids 330-346 of SEQ ID NO: 505. In some embodiments, the endoglin:Crim1 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-25 of SEQ ID NO: 509, and ends at any one of amino acids 148-164 of SEQ ID NO: 509. In some embodiments, the endoglin:Crim1 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 537 or 538. In some embodiments, the endoglin:Crim1 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 35-37 of SEQ ID NO: 537 and ends at any one of amino acids 873-939 of SEQ ID NO: 537. In certain preferred embodiments, endoglin:Crim1 heteromultimers are soluble. In some embodiments, a endoglin:Crim1 heteromultimer of the disclosure binds to one or more TGF-beta superfamily ligands (e.g., binds to one or more TGF-beta superfamily ligands with a K_{D} of at least 1×10⁻⁷). In some embodiments, a endoglin:Crim1 heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a endoglin:Crim1 heteromultimer of the disclosure has a different TGF-beta ligand binding and/or inhibition profile (specificity) compared to a corresponding homomultimer (e.g., endoglin and Crim1 homomultimers). In some embodiments, a endoglin:Crim1 heteromultimer of the disclosure is a heterodimer.

In certain aspects, the disclosure relates to heteromultimers that comprise at least one endoglin polypeptide, which includes fragments, functional variants, and modified forms thereof, and at least one Crim2 polypeptide, which includes fragments, functional variants, and modified forms thereof. In some embodiments, the endoglin:Crim2 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 501, 502, 505, 506, 509, 510, 593, or 594. In some embodiments, the endoglin:Crim2 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-30 of SEQ ID NO: 501 and ends at any one of amino acids 330-346 of SEQ ID NO: 501. In some embodiments, the endoglin:Crim2 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-30 of SEQ ID NO: 505 and ends at any one of amino acids 330-346 of SEQ ID NO: 505. In some embodiments, the endoglin:Crim2 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-25 of SEQ ID NO: 509, and ends at any one of amino acids 148-164 of SEQ ID NO: 509. In some embodiments, the endoglin:Crim2 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 541, 542, 545, or 546. In some embodiments, the endoglin:Crim2 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-138 of SEQ ID NO: 541 and ends at any one of amino acids 1298-1503 of SEQ ID NO: 541. In some embodiments, the endoglin:Crim2 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 24-138 of SEQ ID NO: 545 and ends at any one of amino acids 539-814 of SEQ ID NO: 545. In certain preferred embodiments, endoglin:Crim2 heteromultimers are soluble. In some embodiments, a endoglin:Crim2 heteromultimer of the disclosure binds to one or more TGF-beta superfamily ligands (e.g., binds to one or more TGF-beta superfamily ligands with a K_{D} of at least 1×10⁻⁷). In some embodiments, a endoglin:Crim2 heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a endoglin:Crim2 heteromultimer of the disclosure has a different TGF-beta ligand binding and/or inhibition profile (specificity) compared to a corresponding homomultimer (e.g., endoglin and Crim2 homomultimers). In some embodiments, a endoglin:Crim2 heteromultimer of the disclosure is a heterodimer.

In certain aspects, the disclosure relates to heteromultimers that comprise at least one endoglin polypeptide, which includes fragments, functional variants, and modified forms thereof, and at least one BAMBI polypeptide, which includes fragments, functional variants, and modified forms thereof. In some embodiments, the endoglin:BAMBI heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 501, 502, 505, 506, 509, 510, 593, or 594. In some embodiments, the endoglin:BAMBI heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-30 of SEQ ID NO: 501 and ends at any one of amino acids 330-346 of SEQ ID NO: 501. In some embodiments, the endoglin:BAMBI heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-30 of SEQ ID NO: 505 and ends at any one of amino acids 330-346 of SEQ ID NO: 505. In some embodiments, the endoglin:BAMBI heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-25 of SEQ ID NO: 509, and ends at any one of amino acids 148-164 of SEQ ID NO: 509. In some embodiments, the endoglin:BAMBI heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 549 or 550. In some embodiments, the endoglin:BAMBI heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 21-30 of SEQ ID NO: 549 and ends at any one of amino acids 104-152 of SEQ ID NO: 549. In certain preferred embodiments, endoglin:BAMBI heteromultimers are soluble. In some embodiments, a endoglin:BAMBI heteromultimer of the disclosure binds to one or more TGF-beta superfamily ligands (e.g., binds to one or more TGF-beta superfamily ligands with a K_{D} of at least 1×10⁻⁷). In some embodiments, a endoglin:BAMBI heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a endoglin:BAMBI heteromultimer of the disclosure has a different TGF-beta ligand binding and/or inhibition profile (specificity) compared to a corresponding homomultimer (e.g., endoglin and BAMBI homomultimers). In some embodiments, a endoglin:BAMBI heteromultimer of the disclosure is a heterodimer.

In certain aspects, the disclosure relates to heteromultimers that comprise at least one endoglin polypeptide, which includes fragments, functional variants, and modified forms thereof, and at least one BMPER polypeptide, which includes fragments, functional variants, and modified forms thereof. In some embodiments, the endoglin:BMPER heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 501, 502, 505, 506, 509, 510, 593, or 594. In some embodiments, the endoglin:BMPER heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-30 of SEQ ID NO: 501 and ends at any one of amino acids 330-346 of SEQ ID NO: 501. In some embodiments, the endoglin:BMPER heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-30 of SEQ ID NO: 505 and ends at any one of amino acids 330-346 of SEQ ID NO: 505. In some embodiments, the endoglin:BMPER heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-25 of SEQ ID NO: 509, and ends at any one of amino acids 148-164 of SEQ ID NO: 509. In some embodiments, the endoglin:BMPER heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 553 or 554. In some embodiments, the endoglin:BMPER heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 39-50 of SEQ ID NO: 553 and ends at any one of amino acids 364-369 of SEQ ID NO: 553. In some embodiments, the endoglin:BMPER heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 370-386 of SEQ ID NO: 553 and ends at any one of amino acids 682-685 of SEQ ID NO: 553. In some embodiments, the endoglin:BMPER heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 39-50 of SEQ ID NO: 553 and ends at any one of amino acids 682-685 of SEQ ID NO: 553. In some embodiments, the endoglin:BMPER heteromultimer comprises a BMPER protein, wherein the BMPER protein is a dimer comprising a first polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 39-50 of SEQ ID NO: 553 and ends at any one of amino acids 364-369 of SEQ ID NO: 553, and second polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 370-386 of SEQ ID NO: 553, and ends at any one of amino acids 682-685 of SEQ ID NO: 553. In some embodiments, the endoglin:BMPER heteromultimer comprises a single chain ligand trap that comprises a first BMPER polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 39-50 of SEQ ID NO: 553 and ends at any one of amino acids 364-369 of SEQ ID NO: 553, and second BMPER polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 370-386 of SEQ ID NO: 553 and ends at any one of amino acids 682-685 of SEQ ID NO: 553. In certain preferred embodiments, endoglin:BMPER heteromultimers are soluble. In some embodiments, a endoglin:BMPER heteromultimer of the disclosure binds to one or more TGF-beta superfamily ligands (e.g., binds to one or more TGF-beta superfamily ligands with a K_{D} of at least 1×10⁻⁷). In some embodiments, a endoglin:BMPER heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a endoglin:BMPER heteromultimer of the disclosure has a different TGF-beta ligand binding and/or inhibition profile (specificity) compared to a corresponding homomultimer (e.g., endoglin and BMPER homomultimers). In some embodiments, a endoglin:BMPER heteromultimer of the disclosure is a heterodimer.

In certain aspects, the disclosure relates to heteromultimers that comprise at least one endoglin polypeptide, which includes fragments, functional variants, and modified forms thereof, and at least one RGM-A polypeptide, which includes fragments, functional variants, and modified forms thereof. In some embodiments, the endoglin:RGM-A heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 501, 502, 505, 506, 509, 510, 593, or 594. In some embodiments, the endoglin:RGM-A heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-30 of SEQ ID NO: 501 and ends at any one of amino acids 330-346 of SEQ ID NO: 501. In some embodiments, the endoglin:RGM-A heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-30 of SEQ ID NO: 505 and ends at any one of amino acids 330-346 of SEQ ID NO: 505. In some embodiments, the endoglin:RGM-A heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-25 of SEQ ID NO: 509, and ends at any one of amino acids 148-164 of SEQ ID NO: 509. In some embodiments, the endoglin:RGM-A heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 553 or 554. In some embodiments, the endoglin:RGM-A heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-177 of SEQ ID NO: 561 and ends at any one of amino acids 430-458 of SEQ ID NO: 561. In some embodiments, the endoglin:RGM-A heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-153 of SEQ ID NO: 565 and ends at any one of amino acids 406-434 of SEQ ID NO: 565. In some embodiments, the endoglin:RGM-A heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-169 of SEQ ID NO: 569 and ends at any one of amino acids 422-450 of SEQ ID NO: 569. In certain preferred embodiments, endoglin:RGM-A heteromultimers are soluble. In some embodiments, a endoglin:RGM-A heteromultimer of the disclosure binds to one or more TGF-beta superfamily ligands (e.g., binds to one or more TGF-beta superfamily ligands with a K_{D} of at least 1×10⁻⁷). In some embodiments, a endoglin:RGM-A heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a endoglin:RGM-A heteromultimer of the disclosure has a different TGF-beta ligand binding and/or inhibition profile (specificity) compared to a corresponding homomultimer (e.g., endoglin and RGM-A homomultimers). In some embodiments, a endoglin:RGM-A heteromultimer of the disclosure is a heterodimer.

In certain aspects, the disclosure relates to heteromultimers that comprise at least one endoglin polypeptide, which includes fragments, functional variants, and modified forms thereof, and at least one RGM-B polypeptide, which includes fragments, functional variants, and modified forms thereof. In some embodiments, the endoglin:RGM-B heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 501, 502, 505, 506, 509, 510, 593, or 594. In some embodiments, the endoglin:RGM-B heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-30 of SEQ ID NO: 501 and ends at any one of amino acids 330-346 of SEQ ID NO: 501. In some embodiments, the endoglin:RGM-B heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-30 of SEQ ID NO: 505 and ends at any one of amino acids 330-346 of SEQ ID NO: 505. In some embodiments, the endoglin:RGM-B heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-25 of SEQ ID NO: 509, and ends at any one of amino acids 148-164 of SEQ ID NO: 509. In some embodiments, the endoglin:RGM-B heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 557 or 558. In some embodiments, the endoglin:RGM-B heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-87 of SEQ ID NO: 557 and ends at any one of amino acids 452-478 of SEQ ID NO: 557. In some embodiments, the endoglin:RGM-B heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 210-222 of SEQ ID NO: 557 and ends at any one of amino acids 413-452 of SEQ ID NO: 557. In some embodiments, the endoglin:RGM-B heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 87-95 of SEQ ID NO: 557 and ends at any one of amino acids 204-209 of SEQ ID NO: 557. In some embodiments, the endoglin:RGM-B heteromultimer comprises a RGM-B protein, wherein the RGM-B protein is a dimer comprising a first polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 87-95 of SEQ ID NO: 557 and ends at any one of amino acids 204-209 of SEQ ID NO: 557 and second polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 210-222 of SEQ ID NO: 557 and ends at any one of amino acids 413-452 of SEQ ID NO: 557. In some embodiments, the endoglin:RGM-B heteromultimer comprises a single chain ligand trap that comprises a first RGM-B polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 87-95 of SEQ ID NO: 557 and ends at any one of amino acids 204-209 of SEQ ID NO: 557 and second RGM-B polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 210-222 of SEQ ID NO: 557 and ends at any one of amino acids 413-452 of SEQ ID NO: 557. In certain preferred embodiments, endoglin:RGM-B heteromultimers are soluble. In some embodiments, a endoglin:RGM-B heteromultimer of the disclosure binds to one or more TGF-beta superfamily ligands (e.g., binds to one or more TGF-beta superfamily ligands with a K_{D} of at least 1×10⁻⁷). In some embodiments, a endoglin:RGM-B heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a endoglin:RGM-B heteromultimer of the disclosure has a different TGF-beta ligand binding and/or inhibition profile (specificity) compared to a corresponding homomultimer (e.g., endoglin and RGM-B homomultimers). In some embodiments, a endoglin:RGM-B heteromultimer of the disclosure is a heterodimer.

In certain aspects, the disclosure relates to heteromultimers that comprise at least one endoglin polypeptide, which includes fragments, functional variants, and modified forms thereof, and at least one hemojuvelin polypeptide, which includes fragments, functional variants, and modified forms thereof. In some embodiments, the endoglin:hemojuvelin heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 501, 502, 505, 506, 509, 510, 593, or 594. In some embodiments, the endoglin:hemojuvelin heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-30 of SEQ ID NO: 501 and ends at any one of amino acids 330-346 of SEQ ID NO: 501. In some embodiments, the endoglin:hemojuvelin heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-30 of SEQ ID NO: 505 and ends at any one of amino acids 330-346 of SEQ ID NO: 505. In some embodiments, the endoglin:hemojuvelin heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-25 of SEQ ID NO: 509, and ends at any one of amino acids 148-164 of SEQ ID NO: 509. In some embodiments, the endoglin:hemojuvelin heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 573, 574, 577, 578, 581, or 582. In some embodiments, the endoglin:hemojuvelin heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-36 of SEQ ID NO: 573 and ends at any one of amino acids 400-426 of SEQ ID NO: 573. In some embodiments, the endoglin:hemojuvelin heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 36-42 of SEQ ID NO: 573 and ends at any one of amino acids 167-172 of SEQ ID NO: 573. In some embodiments, the endoglin:hemojuvelin heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 173-185 of SEQ ID NO: 573 and ends at any one of amino acids 361-400 of SEQ ID NO: 573. In some embodiments, the endoglin:hemojuvelin heteromultimer comprises a hemojuvelin protein that is a dimer comprising a first polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 36-42 of SEQ ID NO: 573, and ends at any one of amino acids 167-172 of SEQ ID NO: 573 and second polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 173-185 of SEQ ID NO: 573 and ends at any one of amino acids 361-400 of SEQ ID NO: 573. In some embodiments, the endoglin:hemojuvelin heteromultimer comprises a single chain ligand trap that comprises a first hemojuvelin polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 36-42 of SEQ ID NO: 573 and ends at any one of amino acids 167-172 of SEQ ID NO: 573 and second hemojuvelin polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 173-185 of SEQ ID NO: 573 and ends at any one of amino acids 361-400 of SEQ ID NO: 573. In some embodiments, the endoglin:hemojuvelin heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-6 of SEQ ID NO: 577 and ends at any one of amino acids 287-313 of SEQ ID NO: 577. In some embodiments, the endoglin:hemojuvelin heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-6 of SEQ ID NO: 577 and ends at any one of amino acids 54-59 of SEQ ID NO: 577. In some embodiments, the endoglin:hemojuvelin heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 60-72 of SEQ ID NO: 577 and ends at any one of amino acids 248-287 of SEQ ID NO: 577. In some embodiments, the endoglin:hemojuvelin heteromultimer comprises a hemojuvelin protein, wherein the hemojuvelin protein is a dimer comprising a first polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 1-6 of SEQ ID NO: 577 and ends at any one of amino acids 54-59 of SEQ ID NO: 577, and second polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 60-72 of SEQ ID NO: 577 and ends at any one of amino acids 248-287 of SEQ ID NO: 577. In some embodiments, the endoglin:hemojuvelin heteromultimer comprises a single chain ligand trap that comprises a first hemojuvelin polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 1-6 of SEQ ID NO: 577 and ends at any one of amino acids 54-59 of SEQ ID NO: 577, and second hemojuvelin polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 60-72 of SEQ ID NO: 577, and ends at any one of amino acids 248-287 of SEQ ID NO: 577. In some embodiments, the endoglin:hemojuvelin heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-4 of SEQ ID NO: 581 and ends at any one of amino acids 135-200 of SEQ ID NO: 581. In certain preferred embodiments, endoglin:hemojuvelin heteromultimers are soluble. In some embodiments, a endoglin:hemojuvelin heteromultimer of the disclosure binds to one or more TGF-beta superfamily ligands (e.g., binds to one or more TGF-beta superfamily ligands with a K_{D} of at least 1×10⁻⁷). In some embodiments, a endoglin:hemojuvelin heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a endoglin:hemojuvelin heteromultimer of the disclosure has a different TGF-beta ligand binding and/or inhibition profile (specificity) compared to a corresponding homomultimer (e.g., endoglin and hemojuvelin homomultimers). In some embodiments, a endoglin:hemojuvelin heteromultimer of the disclosure is a heterodimer.

In certain aspects, the disclosure relates to heteromultimers that comprise at least one betaglycan polypeptide, which includes fragments, functional variants, and modified forms thereof, and at least one Cripto-1 polypeptide, which includes fragments, functional variants, and modified forms thereof. In some embodiments, the betaglycan:Cripto-1 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 585, 586, 589, or 590. In some embodiments, the betaglycan:Cripto-1 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 21-28 of SEQ ID NO: 585 and ends at any one of amino acids 381-787 of SEQ ID NO: 585. In some embodiments, the betaglycan:Cripto-1 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 21-28 of SEQ ID NO: 589 and ends at any one of amino acids 380-786 of SEQ ID NO: 589. In some embodiments, the betaglycan:Cripto-1 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 513, 514, 517, or 518. In some embodiments, the betaglycan:Cripto-1 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 31-82 of SEQ ID NO: 513 and ends at any one of amino acids 172-188 of SEQ ID NO: 513. In some embodiments, the betaglycan:Cripto-1 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 15-66 of SEQ ID NO: 517, and ends at any one of amino acids 156-172 of SEQ ID NO: 517. In certain preferred embodiments, betaglycan:Cripto-1 heteromultimers are soluble. In some embodiments, a betaglycan:Cripto-1 heteromultimer of the disclosure binds to one or more TGF-beta superfamily ligands (e.g., binds to one or more TGF-beta superfamily ligands with a K_{D} of at least 1×10⁻⁷). In some embodiments, a betaglycan:Cripto-1 heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a betaglycan:Cripto-1 heteromultimer of the disclosure has a different TGF-beta ligand binding and/or inhibition profile (specificity) compared to a corresponding homomultimer (e.g., betaglycan and Cripto-1 homomultimers). In some embodiments, a betaglycan:Cripto-1 heteromultimer of the disclosure is a heterodimer.

In certain aspects, the disclosure relates to heteromultimers that comprise at least one betaglycan polypeptide, which includes fragments, functional variants, and modified forms thereof, and at least one Cryptic protein polypeptide, which includes fragments, functional variants, and modified forms thereof. In some embodiments, the betaglycan:Cryptic protein heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 585, 586, 589, or 590. In some embodiments, the betaglycan:Cryptic protein heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 21-28 of SEQ ID NO: 585 and ends at any one of amino acids 381-787 of SEQ ID NO: 585. In some embodiments, the betaglycan:Cryptic protein heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 21-28 of SEQ ID NO: 589 and ends at any one of amino acids 380-786 of SEQ ID NO: 589. In some embodiments, the betaglycan:Cryptic protein heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 521, 522, 525, 526, 529, or 530. In some embodiments, the betaglycan:Cryptic protein heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-90 of SEQ ID NO: 521 and ends at any one of amino acids 157-233 of SEQ ID NO: 521. In some embodiments, the betaglycan:Cryptic protein heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-30 of SEQ ID NO: 525 and ends at any one of amino acids 82-191 of SEQ ID NO: 525. In some embodiments, the betaglycan:Cryptic protein heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-30 of SEQ ID NO: 529, and ends at any one of amino acids 82-148 of SEQ ID NO: 529. In certain preferred embodiments, betaglycan:Cryptic protein heteromultimers are soluble. In some embodiments, a betaglycan:Cryptic protein heteromultimer of the disclosure binds to one or more TGF-beta superfamily ligands (e.g., binds to one or more TGF-beta superfamily ligands with a K_{D} of at least 1×10⁻⁷). In some embodiments, a betaglycan:Cryptic protein heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a betaglycan:Cryptic protein heteromultimer of the disclosure has a different TGF-beta ligand binding and/or inhibition profile (specificity) compared to a corresponding homomultimer (e.g., betaglycan and Cryptic protein homomultimers). In some embodiments, a betaglycan:Cryptic protein heteromultimer of the disclosure is a heterodimer.

In certain aspects, the disclosure relates to heteromultimers that comprise at least one betaglycan polypeptide, which includes fragments, functional variants, and modified forms thereof, and at least one Cryptic family protein 1B polypeptide, which includes fragments, functional variants, and modified forms thereof. In some embodiments, the betaglycan:Cryptic family protein 1B heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 585, 586, 589, or 590. In some embodiments, the betaglycan:Cryptic family protein 1B heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 21-28 of SEQ ID NO: 585 and ends at any one of amino acids 381-787 of SEQ ID NO: 585. In some embodiments, the betaglycan:Cryptic family protein 1B heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 21-28 of SEQ ID NO: 589 and ends at any one of amino acids 380-786 of SEQ ID NO: 589. In some embodiments, the betaglycan:Cryptic family protein 1B heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 533 or 534. In some embodiments, the betaglycan:Cryptic family protein 1B heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-30 of SEQ ID NO: 533 and ends at any one of amino acids 82-223 of SEQ ID NO: 533. In certain preferred embodiments, betaglycan:Cryptic family protein 1B heteromultimers are soluble. In some embodiments, a betaglycan:Cryptic family protein 1B heteromultimer of the disclosure binds to one or more TGF-beta superfamily ligands (e.g., binds to one or more TGF-beta superfamily ligands with a K_{D} of at least 1×10⁻⁷). In some embodiments, a betaglycan:Cryptic family protein 1B heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a betaglycan:Cryptic family protein 1B heteromultimer of the disclosure has a different TGF-beta ligand binding and/or inhibition profile (specificity) compared to a corresponding homomultimer (e.g., betaglycan and Cryptic family protein 1B homomultimers). In some embodiments, a betaglycan:Cryptic family protein 1B heteromultimer of the disclosure is a heterodimer.

In certain aspects, the disclosure relates to heteromultimers that comprise at least one betaglycan polypeptide, which includes fragments, functional variants, and modified forms thereof, and at least one Crim1 polypeptide, which includes fragments, functional variants, and modified forms thereof. In some embodiments, the betaglycan:Crim1 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 585, 586, 589, or 590. In some embodiments, the betaglycan:Crim1 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 21-28 of SEQ ID NO: 585 and ends at any one of amino acids 381-787 of SEQ ID NO: 585. In some embodiments, the betaglycan:Crim1 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 21-28 of SEQ ID NO: 589 and ends at any one of amino acids 380-786 of SEQ ID NO: 589. In some embodiments, the betaglycan:Crim1 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 537 or 538. In some embodiments, the betaglycan:Crim1 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 35-37 of SEQ ID NO: 537 and ends at any one of amino acids 873-939 of SEQ ID NO: 537. In certain preferred embodiments, betaglycan:Crim1 heteromultimers are soluble. In some embodiments, a betaglycan:Crim1 heteromultimer of the disclosure binds to one or more TGF-beta superfamily ligands (e.g., binds to one or more TGF-beta superfamily ligands with a K_{D} of at least 1×10⁻⁷). In some embodiments, a betaglycan:Crim1 heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a betaglycan:Crim1 heteromultimer of the disclosure has a different TGF-beta ligand binding and/or inhibition profile (specificity) compared to a corresponding homomultimer (e.g., betaglycan and Crim1 homomultimers). In some embodiments, a betaglycan:Crim1 heteromultimer of the disclosure is a heterodimer.

In certain aspects, the disclosure relates to heteromultimers that comprise at least one betaglycan polypeptide, which includes fragments, functional variants, and modified forms thereof, and at least one Crim2 polypeptide, which includes fragments, functional variants, and modified forms thereof. In some embodiments, the betaglycan:Crim2 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 585, 586, 589, or 590. In some embodiments, the betaglycan:Crim2 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 21-28 of SEQ ID NO: 585 and ends at any one of amino acids 381-787 of SEQ ID NO: 585. In some embodiments, the betaglycan:Crim2 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 21-28 of SEQ ID NO: 589 and ends at any one of amino acids 380-786 of SEQ ID NO: 589. In some embodiments, the betaglycan:Crim2 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 541, 542, 545, or 546. In some embodiments, the betaglycan:Crim2 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-138 of SEQ ID NO: 541 and ends at any one of amino acids 1298-1503 of SEQ ID NO: 541. In some embodiments, the betaglycan:Crim2 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 24-138 of SEQ ID NO: 545 and ends at any one of amino acids 539-814 of SEQ ID NO: 545. In certain preferred embodiments, betaglycan:Crim2 heteromultimers are soluble. In some embodiments, a betaglycan:Crim2 heteromultimer of the disclosure binds to one or more TGF-beta superfamily ligands (e.g., binds to one or more TGF-beta superfamily ligands with a K_{D} of at least 1×10⁻⁷). In some embodiments, a betaglycan:Crim2 heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a betaglycan:Crim2 heteromultimer of the disclosure has a different TGF-beta ligand binding and/or inhibition profile (specificity) compared to a corresponding homomultimer (e.g., betaglycan and Crim2 homomultimers). In some embodiments, a betaglycan:Crim2 heteromultimer of the disclosure is a heterodimer.

In certain aspects, the disclosure relates to heteromultimers that comprise at least one betaglycan polypeptide, which includes fragments, functional variants, and modified forms thereof, and at least one BAMBI polypeptide, which includes fragments, functional variants, and modified forms thereof. In some embodiments, the betaglycan:BAMBI heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 585, 586, 589, or 590. In some embodiments, the betaglycan:BAMBI heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 21-28 of SEQ ID NO: 585 and ends at any one of amino acids 381-787 of SEQ ID NO: 585. In some embodiments, the betaglycan:BAMBI heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 21-28 of SEQ ID NO: 589 and ends at any one of amino acids 380-786 of SEQ ID NO: 589. In some embodiments, the betaglycan:BAMBI heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 549 or 550. In some embodiments, the betaglycan:BAMBI heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 21-30 of SEQ ID NO: 549 and ends at any one of amino acids 104-152 of SEQ ID NO: 549. In certain preferred embodiments, betaglycan:BAMBI heteromultimers are soluble. In some embodiments, a betaglycan:BAMBI heteromultimer of the disclosure binds to one or more TGF-beta superfamily ligands (e.g., binds to one or more TGF-beta superfamily ligands with a K_{D} of at least 1×10⁻⁷). In some embodiments, a betaglycan:BAMBI heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a betaglycan:BAMBI heteromultimer of the disclosure has a different TGF-beta ligand binding and/or inhibition profile (specificity) compared to a corresponding homomultimer (e.g., betaglycan and BAMBI homomultimers). In some embodiments, a betaglycan:BAMBI heteromultimer of the disclosure is a heterodimer.

In certain aspects, the disclosure relates to heteromultimers that comprise at least one betaglycan polypeptide, which includes fragments, functional variants, and modified forms thereof, and at least one BMPER polypeptide, which includes fragments, functional variants, and modified forms thereof. In some embodiments, the betaglycan:BMPER heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 585, 586, 589, or 590. In some embodiments, the betaglycan:BMPER heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 21-28 of SEQ ID NO: 585 and ends at any one of amino acids 381-787 of SEQ ID NO: 585. In some embodiments, the betaglycan:BMPER heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 21-28 of SEQ ID NO: 589 and ends at any one of amino acids 380-786 of SEQ ID NO: 589. In some embodiments, the betaglycan:BMPER heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 553 or 554. In some embodiments, the betaglycan:BMPER heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 39-50 of SEQ ID NO: 553 and ends at any one of amino acids 364-369 of SEQ ID NO: 553. In some embodiments, the betaglycan:BMPER heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 370-386 of SEQ ID NO: 553 and ends at any one of amino acids 682-685 of SEQ ID NO: 553. In some embodiments, the betaglycan:BMPER heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 39-50 of SEQ ID NO: 553 and ends at any one of amino acids 682-685 of SEQ ID NO: 553. In some embodiments, the betaglycan:BMPER heteromultimer comprises a BMPER protein, wherein the BMPER protein is a dimer comprising a first polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 39-50 of SEQ ID NO: 553 and ends at any one of amino acids 364-369 of SEQ ID NO: 553, and second polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 370-386 of SEQ ID NO: 553, and ends at any one of amino acids 682-685 of SEQ ID NO: 553. In some embodiments, the betaglycan:BMPER heteromultimer comprises a single chain ligand trap that comprises a first BMPER polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 39-50 of SEQ ID NO: 553 and ends at any one of amino acids 364-369 of SEQ ID NO: 553, and second BMPER polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 370-386 of SEQ ID NO: 553 and ends at any one of amino acids 682-685 of SEQ ID NO: 553. In certain preferred embodiments, betaglycan:BMPER heteromultimers are soluble. In some embodiments, a betaglycan:BMPER heteromultimer of the disclosure binds to one or more TGF-beta superfamily ligands (e.g., binds to one or more TGF-beta superfamily ligands with a K_{D} of at least 1×10⁻⁷). In some embodiments, a betaglycan:BMPER heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a betaglycan:BMPER heteromultimer of the disclosure has a different TGF-beta ligand binding and/or inhibition profile (specificity) compared to a corresponding homomultimer (e.g., betaglycan and BMPER homomultimers). In some embodiments, a betaglycan:BMPER heteromultimer of the disclosure is a heterodimer.

In certain aspects, the disclosure relates to heteromultimers that comprise at least one betaglycan polypeptide, which includes fragments, functional variants, and modified forms thereof, and at least one RGM-A polypeptide, which includes fragments, functional variants, and modified forms thereof. In some embodiments, the betaglycan:RGM-A heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 585, 586, 589, or 590. In some embodiments, the betaglycan:RGM-A heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 21-28 of SEQ ID NO: 585 and ends at any one of amino acids 381-787 of SEQ ID NO: 585. In some embodiments, the betaglycan:RGM-A heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 21-28 of SEQ ID NO: 589 and ends at any one of amino acids 380-786 of SEQ ID NO: 589. In some embodiments, the betaglycan:RGM-A heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 553 or 554. In some embodiments, the betaglycan:RGM-A heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-177 of SEQ ID NO: 561 and ends at any one of amino acids 430-458 of SEQ ID NO: 561. In some embodiments, the betaglycan:RGM-A heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-153 of SEQ ID NO: 565 and ends at any one of amino acids 406-434 of SEQ ID NO: 565. In some embodiments, the betaglycan:RGM-A heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-169 of SEQ ID NO: 569 and ends at any one of amino acids 422-450 of SEQ ID NO: 569. In certain preferred embodiments, betaglycan:RGM-A heteromultimers are soluble. In some embodiments, a betaglycan:RGM-A heteromultimer of the disclosure binds to one or more TGF-beta superfamily ligands (e.g., binds to one or more TGF-beta superfamily ligands with a K_{D} of at least 1×10⁻⁷). In some embodiments, a betaglycan:RGM-A heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a betaglycan:RGM-A heteromultimer of the disclosure has a different TGF-beta ligand binding and/or inhibition profile (specificity) compared to a corresponding homomultimer (e.g., betaglycan and RGM-A homomultimers). In some embodiments, a betaglycan:RGM-A heteromultimer of the disclosure is a heterodimer.

In certain aspects, the disclosure relates to heteromultimers that comprise at least one betaglycan polypeptide, which includes fragments, functional variants, and modified forms thereof, and at least one RGM-B polypeptide, which includes fragments, functional variants, and modified forms thereof. In some embodiments, the betaglycan:RGM-B heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 585, 586, 589, or 590. In some embodiments, the betaglycan:RGM-B heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 21-28 of SEQ ID NO: 585 and ends at any one of amino acids 381-787 of SEQ ID NO: 585. In some embodiments, the betaglycan:RGM-B heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 21-28 of SEQ ID NO: 589 and ends at any one of amino acids 380-786 of SEQ ID NO: 589. In some embodiments, the betaglycan:RGM-B heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 557 or 558. In some embodiments, the betaglycan:RGM-B heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-87 of SEQ ID NO: 557 and ends at any one of amino acids 452-478 of SEQ ID NO: 557. In some embodiments, the betaglycan:RGM-B heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 210-222 of SEQ ID NO: 557 and ends at any one of amino acids 413-452 of SEQ ID NO: 557. In some embodiments, the betaglycan:RGM-B heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 87-95 of SEQ ID NO: 557 and ends at any one of amino acids 204-209 of SEQ ID NO: 557. In some embodiments, the betaglycan:RGM-B heteromultimer comprises a RGM-B protein, wherein the RGM-B protein is a dimer comprising a first polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 87-95 of SEQ ID NO: 557 and ends at any one of amino acids 204-209 of SEQ ID NO: 557 and second polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 210-222 of SEQ ID NO: 557 and ends at any one of amino acids 413-452 of SEQ ID NO: 557. In some embodiments, the betaglycan:RGM-B heteromultimer comprises a single chain ligand trap that comprises a first RGM-B polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 87-95 of SEQ ID NO: 557 and ends at any one of amino acids 204-209 of SEQ ID NO: 557 and second RGM-B polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 210-222 of SEQ ID NO: 557 and ends at any one of amino acids 413-452 of SEQ ID NO: 557. In certain preferred embodiments, betaglycan:RGM-B heteromultimers are soluble. In some embodiments, a betaglycan:RGM-B heteromultimer of the disclosure binds to one or more TGF-beta superfamily ligands (e.g., binds to one or more TGF-beta superfamily ligands with a K_{D} of at least 1×10⁻⁷). In some embodiments, a betaglycan:RGM-B heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a betaglycan:RGM-B heteromultimer of the disclosure has a different TGF-beta ligand binding and/or inhibition profile (specificity) compared to a corresponding homomultimer (e.g., betaglycan and RGM-B homomultimers). In some embodiments, a betaglycan:RGM-B heteromultimer of the disclosure is a heterodimer.

In certain aspects, the disclosure relates to heteromultimers that comprise at least one betaglycan polypeptide, which includes fragments, functional variants, and modified forms thereof, and at least one hemojuvelin polypeptide, which includes fragments, functional variants, and modified forms thereof. In some embodiments, the betaglycan:hemojuvelin heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 585, 586, 589, or 590. In some embodiments, the betaglycan:hemojuvelin heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 21-28 of SEQ ID NO: 585 and ends at any one of amino acids 381-787 of SEQ ID NO: 585. In some embodiments, the betaglycan:hemojuvelin heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 21-28 of SEQ ID NO: 589 and ends at any one of amino acids 380-786 of SEQ ID NO: 589. In some embodiments, the betaglycan:hemojuvelin heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 573, 574, 577, 578, 581, or 582. In some embodiments, the betaglycan:hemojuvelin heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-36 of SEQ ID NO: 573 and ends at any one of amino acids 400-426 of SEQ ID NO: 573. In some embodiments, the betaglycan:hemojuvelin heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 36-42 of SEQ ID NO: 573 and ends at any one of amino acids 167-172 of SEQ ID NO: 573. In some embodiments, the betaglycan:hemojuvelin heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 173-185 of SEQ ID NO: 573 and ends at any one of amino acids 361-400 of SEQ ID NO: 573. In some embodiments, the betaglycan:hemojuvelin heteromultimer comprises a hemojuvelin protein that is a dimer comprising a first polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 36-42 of SEQ ID NO: 573, and ends at any one of amino acids 167-172 of SEQ ID NO: 573 and second polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 173-185 of SEQ ID NO: 573 and ends at any one of amino acids 361-400 of SEQ ID NO: 573. In some embodiments, the betaglycan:hemojuvelin heteromultimer comprises a single chain ligand trap that comprises a first hemojuvelin polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 36-42 of SEQ ID NO: 573 and ends at any one of amino acids 167-172 of SEQ ID NO: 573 and second hemojuvelin polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 173-185 of SEQ ID NO: 573 and ends at any one of amino acids 361-400 of SEQ ID NO: 573. In some embodiments, the betaglycan:hemojuvelin heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-6 of SEQ ID NO: 577 and ends at any one of amino acids 287-313 of SEQ ID NO: 577. In some embodiments, the betaglycan:hemojuvelin heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-6 of SEQ ID NO: 577 and ends at any one of amino acids 54-59 of SEQ ID NO: 577. In some embodiments, the betaglycan:hemojuvelin heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 60-72 of SEQ ID NO: 577 and ends at any one of amino acids 248-287 of SEQ ID NO: 577. In some embodiments, the betaglycan:hemojuvelin heteromultimer comprises a hemojuvelin protein, wherein the hemojuvelin protein is a dimer comprising a first polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 1-6 of SEQ ID NO: 577 and ends at any one of amino acids 54-59 of SEQ ID NO: 577, and second polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 60-72 of SEQ ID NO: 577 and ends at any one of amino acids 248-287 of SEQ ID NO: 577. In some embodiments, the betaglycan:hemojuvelin heteromultimer comprises a single chain ligand trap that comprises a first hemojuvelin polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 1-6 of SEQ ID NO: 577 and ends at any one of amino acids 54-59 of SEQ ID NO: 577, and second hemojuvelin polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 60-72 of SEQ ID NO: 577, and ends at any one of amino acids 248-287 of SEQ ID NO: 577. In some embodiments, the betaglycan:hemojuvelin heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-4 of SEQ ID NO: 581 and ends at any one of amino acids 135-200 of SEQ ID NO: 581. In certain preferred embodiments, betaglycan:hemojuvelin heteromultimers are soluble. In some embodiments, a betaglycan:hemojuvelin heteromultimer of the disclosure binds to one or more TGF-beta superfamily ligands (e.g., binds to one or more TGF-beta superfamily ligands with a K_{D} of at least 1×10⁻⁷). In some embodiments, a betaglycan:hemojuvelin heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a betaglycan:hemojuvelin heteromultimer of the disclosure has a different TGF-beta ligand binding and/or inhibition profile (specificity) compared to a corresponding homomultimer (e.g., betaglycan and hemojuvelin homomultimers). In some embodiments, a betaglycan:hemojuvelin heteromultimer of the disclosure is a heterodimer.

In certain aspects, the disclosure relates to heteromultimers that comprise at least one Cripto-1 polypeptide, which includes fragments, functional variants, and modified forms thereof, and at least one Cryptic protein polypeptide, which includes fragments, functional variants, and modified forms thereof. In some embodiments, the Cripto-1:Cryptic protein heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 513, 514, 517, or 518. In some embodiments, the Cripto-1:Cryptic protein heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 31-82 of SEQ ID NO: 513 and ends at any one of amino acids 172-188 of SEQ ID NO: 513. In some embodiments, the Cripto-1:Cryptic protein heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 15-66 of SEQ ID NO: 517, and ends at any one of amino acids 156-172 of SEQ ID NO: 517. In some embodiments, the Cripto-1:Cryptic protein heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 521, 522, 525, 526, 529, or 530. In some embodiments, the Cripto-1:Cryptic protein heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-90 of SEQ ID NO: 521 and ends at any one of amino acids 157-233 of SEQ ID NO: 521. In some embodiments, the Cripto-1:Cryptic protein heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-30 of SEQ ID NO: 525 and ends at any one of amino acids 82-191 of SEQ ID NO: 525. In some embodiments, the Cripto-1:Cryptic protein heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-30 of SEQ ID NO: 529, and ends at any one of amino acids 82-148 of SEQ ID NO: 529. In certain preferred embodiments, Cripto-1:Cryptic protein heteromultimers are soluble. In some embodiments, a Cripto-1:Cryptic protein heteromultimer of the disclosure binds to one or more TGF-beta superfamily ligands (e.g., binds to one or more TGF-beta superfamily ligands with a K_{D} of at least 1×10⁻⁷). In some embodiments, a Cripto-1:Cryptic protein heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a Cripto-1:Cryptic protein heteromultimer of the disclosure has a different TGF-beta ligand binding and/or inhibition profile (specificity) compared to a corresponding homomultimer (e.g., Cripto-1 and Cryptic protein homomultimers). In some embodiments, a Cripto-1:Cryptic protein heteromultimer of the disclosure is a heterodimer.

In certain aspects, the disclosure relates to heteromultimers that comprise at least one Cripto-1 polypeptide, which includes fragments, functional variants, and modified forms thereof, and at least one Cryptic family protein 1B polypeptide, which includes fragments, functional variants, and modified forms thereof. In some embodiments, the Cripto-1:Cryptic family protein 1B heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 513, 514, 517, or 518. In some embodiments, the Cripto-1:Cryptic family protein 1B heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 31-82 of SEQ ID NO: 513 and ends at any one of amino acids 172-188 of SEQ ID NO: 513. In some embodiments, the Cripto-1:Cryptic family protein 1B heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 15-66 of SEQ ID NO: 517, and ends at any one of amino acids 156-172 of SEQ ID NO: 517. In some embodiments, the Cripto-1:Cryptic family protein 1B heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 533 or 534. In some embodiments, the Cripto-1:Cryptic family protein 1B heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-30 of SEQ ID NO: 533 and ends at any one of amino acids 82-223 of SEQ ID NO: 533. In certain preferred embodiments, Cripto-1:Cryptic family protein 1B heteromultimers are soluble. In some embodiments, a Cripto-1:Cryptic family protein 1B heteromultimer of the disclosure binds to one or more TGF-beta superfamily ligands (e.g., binds to one or more TGF-beta superfamily ligands with a K_{D} of at least 1×10⁻⁷). In some embodiments, a Cripto-1:Cryptic family protein 1B heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a Cripto-1:Cryptic family protein 1B heteromultimer of the disclosure has a different TGF-beta ligand binding and/or inhibition profile (specificity) compared to a corresponding homomultimer (e.g., Cripto-1 and Cryptic family protein 1B homomultimers). In some embodiments, a Cripto-1:Cryptic family protein 1B heteromultimer of the disclosure is a heterodimer.

In certain aspects, the disclosure relates to heteromultimers that comprise at least one Cripto-1 polypeptide, which includes fragments, functional variants, and modified forms thereof, and at least one Crim1 polypeptide, which includes fragments, functional variants, and modified forms thereof. In some embodiments, the Cripto-1:Crim1 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 513, 514, 517, or 518. In some embodiments, the Cripto-1:Crim1 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 31-82 of SEQ ID NO: 513 and ends at any one of amino acids 172-188 of SEQ ID NO: 513. In some embodiments, the Cripto-1:Crim1 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 15-66 of SEQ ID NO: 517, and ends at any one of amino acids 156-172 of SEQ ID NO: 517. In some embodiments, the Cripto-1:Crim1 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 537 or 538. In some embodiments, the Cripto-1:Crim1 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 35-37 of SEQ ID NO: 537 and ends at any one of amino acids 873-939 of SEQ ID NO: 537. In certain preferred embodiments, Cripto-1:Crim1 heteromultimers are soluble. In some embodiments, a Cripto-1:Crim1 heteromultimer of the disclosure binds to one or more TGF-beta superfamily ligands (e.g., binds to one or more TGF-beta superfamily ligands with a K_{D} of at least 1×10⁻⁷). In some embodiments, a Cripto-1:Crim1 heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a Cripto-1:Crim1 heteromultimer of the disclosure has a different TGF-beta ligand binding and/or inhibition profile (specificity) compared to a corresponding homomultimer (e.g., Cripto-1 and Crim1 homomultimers). In some embodiments, a Cripto-1:Crim1 heteromultimer of the disclosure is a heterodimer.

In certain aspects, the disclosure relates to heteromultimers that comprise at least one Cripto-1 polypeptide, which includes fragments, functional variants, and modified forms thereof, and at least one Crim2 polypeptide, which includes fragments, functional variants, and modified forms thereof. In some embodiments, the Cripto-1:Crim2 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 513, 514, 517, or 518. In some embodiments, the Cripto-1:Crim2 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 31-82 of SEQ ID NO: 513 and ends at any one of amino acids 172-188 of SEQ ID NO: 513. In some embodiments, the Cripto-1:Crim2 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 15-66 of SEQ ID NO: 517, and ends at any one of amino acids 156-172 of SEQ ID NO: 517. In some embodiments, the Cripto-1:Crim2 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 541, 542, 545, or 546. In some embodiments, the Cripto-1:Crim2 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-138 of SEQ ID NO: 541 and ends at any one of amino acids 1298-1503 of SEQ ID NO: 541. In some embodiments, the Cripto-1:Crim2 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 24-138 of SEQ ID NO: 545 and ends at any one of amino acids 539-814 of SEQ ID NO: 545. In certain preferred embodiments, Cripto-1:Crim2 heteromultimers are soluble. In some embodiments, a Cripto-1:Crim2 heteromultimer of the disclosure binds to one or more TGF-beta superfamily ligands (e.g., binds to one or more TGF-beta superfamily ligands with a K_{D} of at least 1×10⁻⁷). In some embodiments, a Cripto-1:Crim2 heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a Cripto-1:Crim2 heteromultimer of the disclosure has a different TGF-beta ligand binding and/or inhibition profile (specificity) compared to a corresponding homomultimer (e.g., Cripto-1 and Crim2 homomultimers). In some embodiments, a Cripto-1:Crim2 heteromultimer of the disclosure is a heterodimer.

In certain aspects, the disclosure relates to heteromultimers that comprise at least one Cripto-1 polypeptide, which includes fragments, functional variants, and modified forms thereof, and at least one BAMBI polypeptide, which includes fragments, functional variants, and modified forms thereof. In some embodiments, the Cripto-1:BAMBI heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 513, 514, 517, or 518. In some embodiments, the Cripto-1:BAMBI heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 31-82 of SEQ ID NO: 513 and ends at any one of amino acids 172-188 of SEQ ID NO: 513. In some embodiments, the Cripto-1:BAMBI heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 15-66 of SEQ ID NO: 517, and ends at any one of amino acids 156-172 of SEQ ID NO: 517. In some embodiments, the Cripto-1:BAMBI heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 549 or 550. In some embodiments, the Cripto-1:BAMBI heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 21-30 of SEQ ID NO: 549 and ends at any one of amino acids 104-152 of SEQ ID NO: 549. In certain preferred embodiments, Cripto-1:BAMBI heteromultimers are soluble. In some embodiments, a Cripto-1:BAMBI heteromultimer of the disclosure binds to one or more TGF-beta superfamily ligands (e.g., binds to one or more TGF-beta superfamily ligands with a K_{D} of at least 1×10⁻⁷). In some embodiments, a Cripto-1:BAMBI heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a Cripto-1:BAMBI heteromultimer of the disclosure has a different TGF-beta ligand binding and/or inhibition profile (specificity) compared to a corresponding homomultimer (e.g., Cripto-1 and BAMBI homomultimers). In some embodiments, a Cripto-1:BAMBI heteromultimer of the disclosure is a heterodimer.

In certain aspects, the disclosure relates to heteromultimers that comprise at least one Cripto-1 polypeptide, which includes fragments, functional variants, and modified forms thereof, and at least one BMPER polypeptide, which includes fragments, functional variants, and modified forms thereof. In some embodiments, the Cripto-1:BMPER heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 513, 514, 517, or 518. In some embodiments, the Cripto-1:BMPER heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 31-82 of SEQ ID NO: 513 and ends at any one of amino acids 172-188 of SEQ ID NO: 513. In some embodiments, the Cripto-1:BMPER heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 15-66 of SEQ ID NO: 517, and ends at any one of amino acids 156-172 of SEQ ID NO: 517. In some embodiments, the Cripto-1:BMPER heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 553 or 554. In some embodiments, the Cripto-1:BMPER heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 39-50 of SEQ ID NO: 553 and ends at any one of amino acids 364-369 of SEQ ID NO: 553. In some embodiments, the Cripto-1:BMPER heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 370-386 of SEQ ID NO: 553 and ends at any one of amino acids 682-685 of SEQ ID NO: 553. In some embodiments, the Cripto-1:BMPER heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 39-50 of SEQ ID NO: 553 and ends at any one of amino acids 682-685 of SEQ ID NO: 553. In some embodiments, the Cripto-1:BMPER heteromultimer comprises a BMPER protein, wherein the BMPER protein is a dimer comprising a first polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 39-50 of SEQ ID NO: 553 and ends at any one of amino acids 364-369 of SEQ ID NO: 553, and second polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 370-386 of SEQ ID NO: 553, and ends at any one of amino acids 682-685 of SEQ ID NO: 553. In some embodiments, the Cripto-1:BMPER heteromultimer comprises a single chain ligand trap that comprises a first BMPER polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 39-50 of SEQ ID NO: 553 and ends at any one of amino acids 364-369 of SEQ ID NO: 553, and second BMPER polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 370-386 of SEQ ID NO: 553 and ends at any one of amino acids 682-685 of SEQ ID NO: 553. In certain preferred embodiments, Cripto-1:BMPER heteromultimers are soluble. In some embodiments, a Cripto-1:BMPER heteromultimer of the disclosure binds to one or more TGF-beta superfamily ligands (e.g., binds to one or more TGF-beta superfamily ligands with a K_{D} of at least 1×10⁻⁷). In some embodiments, a Cripto-1:BMPER heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a Cripto-1:BMPER heteromultimer of the disclosure has a different TGF-beta ligand binding and/or inhibition profile (specificity) compared to a corresponding homomultimer (e.g., Cripto-1 and BMPER homomultimers). In some embodiments, a Cripto-1:BMPER heteromultimer of the disclosure is a heterodimer.

In certain aspects, the disclosure relates to heteromultimers that comprise at least one Cripto-1 polypeptide, which includes fragments, functional variants, and modified forms thereof, and at least one RGM-A polypeptide, which includes fragments, functional variants, and modified forms thereof. In some embodiments, the Cripto-1:RGM-A heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 513, 514, 517, or 518. In some embodiments, the Cripto-1:RGM-A heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 31-82 of SEQ ID NO: 513 and ends at any one of amino acids 172-188 of SEQ ID NO: 513. In some embodiments, the Cripto-1:RGM-A heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 15-66 of SEQ ID NO: 517, and ends at any one of amino acids 156-172 of SEQ ID NO: 517. In some embodiments, the Cripto-1:RGM-A heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 553 or 554. In some embodiments, the Cripto-1:RGM-A heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-177 of SEQ ID NO: 561 and ends at any one of amino acids 430-458 of SEQ ID NO: 561. In some embodiments, the Cripto-1:RGM-A heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-153 of SEQ ID NO: 565 and ends at any one of amino acids 406-434 of SEQ ID NO: 565. In some embodiments, the Cripto-1:RGM-A heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-169 of SEQ ID NO: 569 and ends at any one of amino acids 422-450 of SEQ ID NO: 569. In certain preferred embodiments, Cripto-1:RGM-A heteromultimers are soluble. In some embodiments, a Cripto-1:RGM-A heteromultimer of the disclosure binds to one or more TGF-beta superfamily ligands (e.g., binds to one or more TGF-beta superfamily ligands with a K_{D} of at least 1×10⁻⁷). In some embodiments, a Cripto-1:RGM-A heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a Cripto-1:RGM-A heteromultimer of the disclosure has a different TGF-beta ligand binding and/or inhibition profile (specificity) compared to a corresponding homomultimer (e.g., Cripto-1 and RGM-A homomultimers). In some embodiments, a Cripto-1:RGM-A heteromultimer of the disclosure is a heterodimer.

In certain aspects, the disclosure relates to heteromultimers that comprise at least one Cripto-1 polypeptide, which includes fragments, functional variants, and modified forms thereof, and at least one RGM-B polypeptide, which includes fragments, functional variants, and modified forms thereof. In some embodiments, the Cripto-1:RGM-B heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 513, 514, 517, or 518. In some embodiments, the Cripto-1:RGM-B heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 31-82 of SEQ ID NO: 513 and ends at any one of amino acids 172-188 of SEQ ID NO: 513. In some embodiments, the Cripto-1:RGM-B heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 15-66 of SEQ ID NO: 517, and ends at any one of amino acids 156-172 of SEQ ID NO: 517. In some embodiments, the Cripto-1:RGM-B heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 557 or 558. In some embodiments, the Cripto-1:RGM-B heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-87 of SEQ ID NO: 557 and ends at any one of amino acids 452-478 of SEQ ID NO: 557. In some embodiments, the Cripto-1:RGM-B heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 210-222 of SEQ ID NO: 557 and ends at any one of amino acids 413-452 of SEQ ID NO: 557. In some embodiments, the Cripto-1:RGM-B heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 87-95 of SEQ ID NO: 557 and ends at any one of amino acids 204-209 of SEQ ID NO: 557. In some embodiments, the Cripto-1:RGM-B heteromultimer comprises a RGM-B protein, wherein the RGM-B protein is a dimer comprising a first polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 87-95 of SEQ ID NO: 557 and ends at any one of amino acids 204-209 of SEQ ID NO: 557 and second polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 210-222 of SEQ ID NO: 557 and ends at any one of amino acids 413-452 of SEQ ID NO: 557. In some embodiments, the Cripto-1:RGM-B heteromultimer comprises a single chain ligand trap that comprises a first RGM-B polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 87-95 of SEQ ID NO: 557 and ends at any one of amino acids 204-209 of SEQ ID NO: 557 and second RGM-B polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 210-222 of SEQ ID NO: 557 and ends at any one of amino acids 413-452 of SEQ ID NO: 557. In certain preferred embodiments, Cripto-1:RGM-B heteromultimers are soluble. In some embodiments, a Cripto-1:RGM-B heteromultimer of the disclosure binds to one or more TGF-beta superfamily ligands (e.g., binds to one or more TGF-beta superfamily ligands with a K_{D} of at least 1×10⁻⁷). In some embodiments, a Cripto-1:RGM-B heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a Cripto-1:RGM-B heteromultimer of the disclosure has a different TGF-beta ligand binding and/or inhibition profile (specificity) compared to a corresponding homomultimer (e.g., Cripto-1 and RGM-B homomultimers). In some embodiments, a Cripto-1:RGM-B heteromultimer of the disclosure is a heterodimer.

In certain aspects, the disclosure relates to heteromultimers that comprise at least one Cripto-1 polypeptide, which includes fragments, functional variants, and modified forms thereof, and at least one hemojuvelin polypeptide, which includes fragments, functional variants, and modified forms thereof. In some embodiments, the Cripto-1:hemojuvelin heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 513, 514, 517, or 518. In some embodiments, the Cripto-1:hemojuvelin heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 31-82 of SEQ ID NO: 513 and ends at any one of amino acids 172-188 of SEQ ID NO: 513. In some embodiments, the Cripto-1:hemojuvelin heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 15-66 of SEQ ID NO: 517, and ends at any one of amino acids 156-172 of SEQ ID NO: 517. In some embodiments, the Cripto-1:hemojuvelin heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 573, 574, 577, 578, 581, or 582. In some embodiments, the Cripto-1:hemojuvelin heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-36 of SEQ ID NO: 573 and ends at any one of amino acids 400-426 of SEQ ID NO: 573. In some embodiments, the Cripto-1:hemojuvelin heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 36-42 of SEQ ID NO: 573 and ends at any one of amino acids 167-172 of SEQ ID NO: 573. In some embodiments, the Cripto-1:hemojuvelin heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 173-185 of SEQ ID NO: 573 and ends at any one of amino acids 361-400 of SEQ ID NO: 573. In some embodiments, the Cripto-1:hemojuvelin heteromultimer comprises a hemojuvelin protein that is a dimer comprising a first polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 36-42 of SEQ ID NO: 573, and ends at any one of amino acids 167-172 of SEQ ID NO: 573 and second polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 173-185 of SEQ ID NO: 573 and ends at any one of amino acids 361-400 of SEQ ID NO: 573. In some embodiments, the Cripto-1:hemojuvelin heteromultimer comprises a single chain ligand trap that comprises a first hemojuvelin polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 36-42 of SEQ ID NO: 573 and ends at any one of amino acids 167-172 of SEQ ID NO: 573 and second hemojuvelin polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 173-185 of SEQ ID NO: 573 and ends at any one of amino acids 361-400 of SEQ ID NO: 573. In some embodiments, the Cripto-1:hemojuvelin heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-6 of SEQ ID NO: 577 and ends at any one of amino acids 287-313 of SEQ ID NO: 577. In some embodiments, the Cripto-1:hemojuvelin heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-6 of SEQ ID NO: 577 and ends at any one of amino acids 54-59 of SEQ ID NO: 577. In some embodiments, the Cripto-1:hemojuvelin heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 60-72 of SEQ ID NO: 577 and ends at any one of amino acids 248-287 of SEQ ID NO: 577. In some embodiments, the Cripto-1:hemojuvelin heteromultimer comprises a hemojuvelin protein, wherein the hemojuvelin protein is a dimer comprising a first polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 1-6 of SEQ ID NO: 577 and ends at any one of amino acids 54-59 of SEQ ID NO: 577, and second polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 60-72 of SEQ ID NO: 577 and ends at any one of amino acids 248-287 of SEQ ID NO: 577. In some embodiments, the Cripto-1:hemojuvelin heteromultimer comprises a single chain ligand trap that comprises a first hemojuvelin polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 1-6 of SEQ ID NO: 577 and ends at any one of amino acids 54-59 of SEQ ID NO: 577, and second hemojuvelin polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 60-72 of SEQ ID NO: 577, and ends at any one of amino acids 248-287 of SEQ ID NO: 577. In some embodiments, the Cripto-1:hemojuvelin heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-4 of SEQ ID NO: 581 and ends at any one of amino acids 135-200 of SEQ ID NO: 581. In certain preferred embodiments, Cripto-1:hemojuvelin heteromultimers are soluble. In some embodiments, a Cripto-1:hemojuvelin heteromultimer of the disclosure binds to one or more TGF-beta superfamily ligands (e.g., binds to one or more TGF-beta superfamily ligands with a K_{D} of at least 1×10⁻⁷). In some embodiments, a Cripto-1:hemojuvelin heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a Cripto-1:hemojuvelin heteromultimer of the disclosure has a different TGF-beta ligand binding and/or inhibition profile (specificity) compared to a corresponding homomultimer (e.g., Cripto-1 and hemojuvelin homomultimers). In some embodiments, a Cripto-1:hemojuvelin heteromultimer of the disclosure is a heterodimer.

In certain aspects, the disclosure relates to heteromultimers that comprise at least one Cryptic protein polypeptide, which includes fragments, functional variants, and modified forms thereof, and at least one Cryptic family protein 1B polypeptide, which includes fragments, functional variants, and modified forms thereof. In some embodiments, the Cryptic protein:Cryptic family protein 1B heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 521, 522, 525, 526, 529, or 530. In some embodiments, the Cryptic protein:Cryptic family protein 1B heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-90 of SEQ ID NO: 521 and ends at any one of amino acids 157-233 of SEQ ID NO: 521. In some embodiments, the Cryptic protein:Cryptic family protein 1B heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-30 of SEQ ID NO: 525 and ends at any one of amino acids 82-191 of SEQ ID NO: 525. In some embodiments, the Cryptic protein:Cryptic family protein 1B heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-30 of SEQ ID NO: 529, and ends at any one of amino acids 82-148 of SEQ ID NO: 529. In some embodiments, the Cryptic protein:Cryptic family protein 1B heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 533 or 534. In some embodiments, the Cryptic protein:Cryptic family protein 1B heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-30 of SEQ ID NO: 533 and ends at any one of amino acids 82-223 of SEQ ID NO: 533. In certain preferred embodiments, Cryptic protein:Cryptic family protein 1B heteromultimers are soluble. In some embodiments, a Cryptic protein:Cryptic family protein 1B heteromultimer of the disclosure binds to one or more TGF-beta superfamily ligands (e.g., binds to one or more TGF-beta superfamily ligands with a K_{D} of at least 1×10⁻⁷). In some embodiments, a Cryptic protein:Cryptic family protein 1B heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a Cryptic protein:Cryptic family protein 1B heteromultimer of the disclosure has a different TGF-beta ligand binding and/or inhibition profile (specificity) compared to a corresponding homomultimer (e.g., Cryptic protein and Cryptic family protein 1B homomultimers). In some embodiments, a Cryptic protein:Cryptic family protein 1B heteromultimer of the disclosure is a heterodimer.

In certain aspects, the disclosure relates to heteromultimers that comprise at least one Cryptic protein polypeptide, which includes fragments, functional variants, and modified forms thereof, and at least one Crim1 polypeptide, which includes fragments, functional variants, and modified forms thereof. In some embodiments, the Cryptic protein:Crim1 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 521, 522, 525, 526, 529, or 530. In some embodiments, the Cryptic protein:Crim1 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-90 of SEQ ID NO: 521 and ends at any one of amino acids 157-233 of SEQ ID NO: 521. In some embodiments, the Cryptic protein:Crim1 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-30 of SEQ ID NO: 525 and ends at any one of amino acids 82-191 of SEQ ID NO: 525. In some embodiments, the Cryptic protein:Crim1 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-30 of SEQ ID NO: 529, and ends at any one of amino acids 82-148 of SEQ ID NO: 529. In some embodiments, the Cryptic protein:Crim1 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 537 or 538. In some embodiments, the Cryptic protein:Crim1 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 35-37 of SEQ ID NO: 537 and ends at any one of amino acids 873-939 of SEQ ID NO: 537. In certain preferred embodiments, Cryptic protein:Crim1 heteromultimers are soluble. In some embodiments, a Cryptic protein:Crim1 heteromultimer of the disclosure binds to one or more TGF-beta superfamily ligands (e.g., binds to one or more TGF-beta superfamily ligands with a K_{D} of at least 1×10⁻⁷). In some embodiments, a Cryptic protein:Crim1 heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a Cryptic protein:Crim1 heteromultimer of the disclosure has a different TGF-beta ligand binding and/or inhibition profile (specificity) compared to a corresponding homomultimer (e.g., Cryptic protein and Crim1 homomultimers). In some embodiments, a Cryptic protein:Crim1 heteromultimer of the disclosure is a heterodimer.

In certain aspects, the disclosure relates to heteromultimers that comprise at least one Cryptic protein polypeptide, which includes fragments, functional variants, and modified forms thereof, and at least one Crim2 polypeptide, which includes fragments, functional variants, and modified forms thereof. In some embodiments, the Cryptic protein:Crim2 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 521, 522, 525, 526, 529, or 530. In some embodiments, the Cryptic protein:Crim2 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-90 of SEQ ID NO: 521 and ends at any one of amino acids 157-233 of SEQ ID NO: 521. In some embodiments, the Cryptic protein:Crim2 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-30 of SEQ ID NO: 525 and ends at any one of amino acids 82-191 of SEQ ID NO: 525. In some embodiments, the Cryptic protein:Crim2 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-30 of SEQ ID NO: 529, and ends at any one of amino acids 82-148 of SEQ ID NO: 529. In some embodiments, the Cryptic protein:Crim2 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 541, 542, 545, or 546. In some embodiments, the Cryptic protein:Crim2 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-138 of SEQ ID NO: 541 and ends at any one of amino acids 1298-1503 of SEQ ID NO: 541. In some embodiments, the Cryptic protein:Crim2 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 24-138 of SEQ ID NO: 545 and ends at any one of amino acids 539-814 of SEQ ID NO: 545. In certain preferred embodiments, Cryptic protein:Crim2 heteromultimers are soluble. In some embodiments, a Cryptic protein:Crim2 heteromultimer of the disclosure binds to one or more TGF-beta superfamily ligands (e.g., binds to one or more TGF-beta superfamily ligands with a K_{D} of at least 1×10⁻⁷). In some embodiments, a Cryptic protein:Crim2 heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a Cryptic protein:Crim2 heteromultimer of the disclosure has a different TGF-beta ligand binding and/or inhibition profile (specificity) compared to a corresponding homomultimer (e.g., Cryptic protein and Crim2 homomultimers). In some embodiments, a Cryptic protein:Crim2 heteromultimer of the disclosure is a heterodimer.

In certain aspects, the disclosure relates to heteromultimers that comprise at least one Cryptic protein polypeptide, which includes fragments, functional variants, and modified forms thereof, and at least one BAMBI polypeptide, which includes fragments, functional variants, and modified forms thereof. In some embodiments, the Cryptic protein:BAMBI heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 521, 522, 525, 526, 529, or 530. In some embodiments, the Cryptic protein:BAMBI heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-90 of SEQ ID NO: 521 and ends at any one of amino acids 157-233 of SEQ ID NO: 521. In some embodiments, the Cryptic protein:BAMBI heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-30 of SEQ ID NO: 525 and ends at any one of amino acids 82-191 of SEQ ID NO: 525. In some embodiments, the Cryptic protein:BAMBI heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-30 of SEQ ID NO: 529, and ends at any one of amino acids 82-148 of SEQ ID NO: 529. In some embodiments, the Cryptic protein:BAMBI heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 549 or 550. In some embodiments, the Cryptic protein:BAMBI heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 21-30 of SEQ ID NO: 549 and ends at any one of amino acids 104-152 of SEQ ID NO: 549. In certain preferred embodiments, Cryptic protein:BAMBI heteromultimers are soluble. In some embodiments, a Cryptic protein:BAMBI heteromultimer of the disclosure binds to one or more TGF-beta superfamily ligands (e.g., binds to one or more TGF-beta superfamily ligands with a K_{D} of at least 1×10⁻⁷). In some embodiments, a Cryptic protein:BAMBI heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a Cryptic protein:BAMBI heteromultimer of the disclosure has a different TGF-beta ligand binding and/or inhibition profile (specificity) compared to a corresponding homomultimer (e.g., Cryptic protein and BAMBI homomultimers). In some embodiments, a Cryptic protein:BAMBI heteromultimer of the disclosure is a heterodimer.

In certain aspects, the disclosure relates to heteromultimers that comprise at least one Cryptic protein polypeptide, which includes fragments, functional variants, and modified forms thereof, and at least one BMPER polypeptide, which includes fragments, functional variants, and modified forms thereof. In some embodiments, the Cryptic protein:BMPER heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 521, 522, 525, 526, 529, or 530. In some embodiments, the Cryptic protein:BMPER heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-90 of SEQ ID NO: 521 and ends at any one of amino acids 157-233 of SEQ ID NO: 521. In some embodiments, the Cryptic protein:BMPER heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-30 of SEQ ID NO: 525 and ends at any one of amino acids 82-191 of SEQ ID NO: 525. In some embodiments, the Cryptic protein:BMPER heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-30 of SEQ ID NO: 529, and ends at any one of amino acids 82-148 of SEQ ID NO: 529. In some embodiments, the Cryptic protein:BMPER heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 553 or 554. In some embodiments, the Cryptic protein:BMPER heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 39-50 of SEQ ID NO: 553 and ends at any one of amino acids 364-369 of SEQ ID NO: 553. In some embodiments, the Cryptic protein:BMPER heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 370-386 of SEQ ID NO: 553 and ends at any one of amino acids 682-685 of SEQ ID NO: 553. In some embodiments, the Cryptic protein:BMPER heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 39-50 of SEQ ID NO: 553 and ends at any one of amino acids 682-685 of SEQ ID NO: 553. In some embodiments, the Cryptic protein:BMPER heteromultimer comprises a BMPER protein, wherein the BMPER protein is a dimer comprising a first polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 39-50 of SEQ ID NO: 553 and ends at any one of amino acids 364-369 of SEQ ID NO: 553, and second polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 370-386 of SEQ ID NO: 553, and ends at any one of amino acids 682-685 of SEQ ID NO: 553. In some embodiments, the Cryptic protein:BMPER heteromultimer comprises a single chain ligand trap that comprises a first BMPER polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 39-50 of SEQ ID NO: 553 and ends at any one of amino acids 364-369 of SEQ ID NO: 553, and second BMPER polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 370-386 of SEQ ID NO: 553 and ends at any one of amino acids 682-685 of SEQ ID NO: 553. In certain preferred embodiments, Cryptic protein:BMPER heteromultimers are soluble. In some embodiments, a Cryptic protein:BMPER heteromultimer of the disclosure binds to one or more TGF-beta superfamily ligands (e.g., binds to one or more TGF-beta superfamily ligands with a K_{D} of at least 1×10⁻⁷). In some embodiments, a Cryptic protein:BMPER heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a Cryptic protein:BMPER heteromultimer of the disclosure has a different TGF-beta ligand binding and/or inhibition profile (specificity) compared to a corresponding homomultimer (e.g., Cryptic protein and BMPER homomultimers). In some embodiments, a Cryptic protein:BMPER heteromultimer of the disclosure is a heterodimer.

In certain aspects, the disclosure relates to heteromultimers that comprise at least one Cryptic protein polypeptide, which includes fragments, functional variants, and modified forms thereof, and at least one RGM-A polypeptide, which includes fragments, functional variants, and modified forms thereof. In some embodiments, the Cryptic protein:RGM-A heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 521, 522, 525, 526, 529, or 530. In some embodiments, the Cryptic protein:RGM-A heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-90 of SEQ ID NO: 521 and ends at any one of amino acids 157-233 of SEQ ID NO: 521. In some embodiments, the Cryptic protein:RGM-A heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-30 of SEQ ID NO: 525 and ends at any one of amino acids 82-191 of SEQ ID NO: 525. In some embodiments, the Cryptic protein:RGM-A heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-30 of SEQ ID NO: 529, and ends at any one of amino acids 82-148 of SEQ ID NO: 529. In some embodiments, the Cryptic protein:RGM-A heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 553 or 554. In some embodiments, the Cryptic protein:RGM-A heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-177 of SEQ ID NO: 561 and ends at any one of amino acids 430-458 of SEQ ID NO: 561. In some embodiments, the Cryptic protein:RGM-A heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-153 of SEQ ID NO: 565 and ends at any one of amino acids 406-434 of SEQ ID NO: 565. In some embodiments, the Cryptic protein:RGM-A heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-169 of SEQ ID NO: 569 and ends at any one of amino acids 422-450 of SEQ ID NO: 569. In certain preferred embodiments, Cryptic protein:RGM-A heteromultimers are soluble. In some embodiments, a Cryptic protein:RGM-A heteromultimer of the disclosure binds to one or more TGF-beta superfamily ligands (e.g., binds to one or more TGF-beta superfamily ligands with a K_{D} of at least 1×10⁻⁷). In some embodiments, a Cryptic protein:RGM-A heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a Cryptic protein:RGM-A heteromultimer of the disclosure has a different TGF-beta ligand binding and/or inhibition profile (specificity) compared to a corresponding homomultimer (e.g., Cryptic protein and RGM-A homomultimers). In some embodiments, a Cryptic protein:RGM-A heteromultimer of the disclosure is a heterodimer.

In certain aspects, the disclosure relates to heteromultimers that comprise at least one Cryptic protein polypeptide, which includes fragments, functional variants, and modified forms thereof, and at least one RGM-B polypeptide, which includes fragments, functional variants, and modified forms thereof. In some embodiments, the Cryptic protein:RGM-B heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 521, 522, 525, 526, 529, or 530. In some embodiments, the Cryptic protein:RGM-B heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-90 of SEQ ID NO: 521 and ends at any one of amino acids 157-233 of SEQ ID NO: 521. In some embodiments, the Cryptic protein:RGM-B heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-30 of SEQ ID NO: 525 and ends at any one of amino acids 82-191 of SEQ ID NO: 525. In some embodiments, the Cryptic protein:RGM-B heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-30 of SEQ ID NO: 529, and ends at any one of amino acids 82-148 of SEQ ID NO: 529. In some embodiments, the Cryptic protein:RGM-B heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 557 or 558. In some embodiments, the Cryptic protein:RGM-B heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-87 of SEQ ID NO: 557 and ends at any one of amino acids 452-478 of SEQ ID NO: 557. In some embodiments, the Cryptic protein:RGM-B heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 210-222 of SEQ ID NO: 557 and ends at any one of amino acids 413-452 of SEQ ID NO: 557. In some embodiments, the Cryptic protein:RGM-B heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 87-95 of SEQ ID NO: 557 and ends at any one of amino acids 204-209 of SEQ ID NO: 557. In some embodiments, the Cryptic protein:RGM-B heteromultimer comprises a RGM-B protein, wherein the RGM-B protein is a dimer comprising a first polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 87-95 of SEQ ID NO: 557 and ends at any one of amino acids 204-209 of SEQ ID NO: 557 and second polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 210-222 of SEQ ID NO: 557 and ends at any one of amino acids 413-452 of SEQ ID NO: 557. In some embodiments, the Cryptic protein:RGM-B heteromultimer comprises a single chain ligand trap that comprises a first RGM-B polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 87-95 of SEQ ID NO: 557 and ends at any one of amino acids 204-209 of SEQ ID NO: 557 and second RGM-B polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 210-222 of SEQ ID NO: 557 and ends at any one of amino acids 413-452 of SEQ ID NO: 557. In certain preferred embodiments, Cryptic protein:RGM-B heteromultimers are soluble. In some embodiments, a Cryptic protein:RGM-B heteromultimer of the disclosure binds to one or more TGF-beta superfamily ligands (e.g., binds to one or more TGF-beta superfamily ligands with a K_{D} of at least 1×10⁻⁷). In some embodiments, a Cryptic protein:RGM-B heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a Cryptic protein:RGM-B heteromultimer of the disclosure has a different TGF-beta ligand binding and/or inhibition profile (specificity) compared to a corresponding homomultimer (e.g., Cryptic protein and RGM-B homomultimers). In some embodiments, a Cryptic protein:RGM-B heteromultimer of the disclosure is a heterodimer.

In certain aspects, the disclosure relates to heteromultimers that comprise at least one Cryptic protein polypeptide, which includes fragments, functional variants, and modified forms thereof, and at least one hemojuvelin polypeptide, which includes fragments, functional variants, and modified forms thereof. In some embodiments, the Cryptic protein:hemojuvelin heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 521, 522, 525, 526, 529, or 530. In some embodiments, the Cryptic protein:hemojuvelin heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-90 of SEQ ID NO: 521 and ends at any one of amino acids 157-233 of SEQ ID NO: 521. In some embodiments, the Cryptic protein:hemojuvelin heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-30 of SEQ ID NO: 525 and ends at any one of amino acids 82-191 of SEQ ID NO: 525. In some embodiments, the Cryptic protein:hemojuvelin heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-30 of SEQ ID NO: 529, and ends at any one of amino acids 82-148 of SEQ ID NO: 529. In some embodiments, the Cryptic protein:hemojuvelin heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 573, 574, 577, 578, 581, or 582. In some embodiments, the Cryptic protein:hemojuvelin heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-36 of SEQ ID NO: 573 and ends at any one of amino acids 400-426 of SEQ ID NO: 573. In some embodiments, the Cryptic protein:hemojuvelin heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 36-42 of SEQ ID NO: 573 and ends at any one of amino acids 167-172 of SEQ ID NO: 573. In some embodiments, the Cryptic protein:hemojuvelin heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 173-185 of SEQ ID NO: 573 and ends at any one of amino acids 361-400 of SEQ ID NO: 573. In some embodiments, the Cryptic protein:hemojuvelin heteromultimer comprises a hemojuvelin protein that is a dimer comprising a first polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 36-42 of SEQ ID NO: 573, and ends at any one of amino acids 167-172 of SEQ ID NO: 573 and second polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 173-185 of SEQ ID NO: 573 and ends at any one of amino acids 361-400 of SEQ ID NO: 573. In some embodiments, the Cryptic protein:hemojuvelin heteromultimer comprises a single chain ligand trap that comprises a first hemojuvelin polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 36-42 of SEQ ID NO: 573 and ends at any one of amino acids 167-172 of SEQ ID NO: 573 and second hemojuvelin polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 173-185 of SEQ ID NO: 573 and ends at any one of amino acids 361-400 of SEQ ID NO: 573. In some embodiments, the Cryptic protein:hemojuvelin heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-6 of SEQ ID NO: 577 and ends at any one of amino acids 287-313 of SEQ ID NO: 577. In some embodiments, the Cryptic protein:hemojuvelin heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-6 of SEQ ID NO: 577 and ends at any one of amino acids 54-59 of SEQ ID NO: 577. In some embodiments, the Cryptic protein:hemojuvelin heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 60-72 of SEQ ID NO: 577 and ends at any one of amino acids 248-287 of SEQ ID NO: 577. In some embodiments, the Cryptic protein:hemojuvelin heteromultimer comprises a hemojuvelin protein, wherein the hemojuvelin protein is a dimer comprising a first polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 1-6 of SEQ ID NO: 577 and ends at any one of amino acids 54-59 of SEQ ID NO: 577, and second polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 60-72 of SEQ ID NO: 577 and ends at any one of amino acids 248-287 of SEQ ID NO: 577. In some embodiments, the Cryptic protein:hemojuvelin heteromultimer comprises a single chain ligand trap that comprises a first hemojuvelin polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 1-6 of SEQ ID NO: 577 and ends at any one of amino acids 54-59 of SEQ ID NO: 577, and second hemojuvelin polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 60-72 of SEQ ID NO: 577, and ends at any one of amino acids 248-287 of SEQ ID NO: 577. In some embodiments, the Cryptic protein:hemojuvelin heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-4 of SEQ ID NO: 581 and ends at any one of amino acids 135-200 of SEQ ID NO: 581. In certain preferred embodiments, Cryptic protein:hemojuvelin heteromultimers are soluble. In some embodiments, a Cryptic protein:hemojuvelin heteromultimer of the disclosure binds to one or more TGF-beta superfamily ligands (e.g., binds to one or more TGF-beta superfamily ligands with a K_{D} of at least 1×10⁻⁷). In some embodiments, a Cryptic protein:hemojuvelin heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a Cryptic protein:hemojuvelin heteromultimer of the disclosure has a different TGF-beta ligand binding and/or inhibition profile (specificity) compared to a corresponding homomultimer (e.g., Cryptic protein and hemojuvelin homomultimers). In some embodiments, a Cryptic protein:hemojuvelin heteromultimer of the disclosure is a heterodimer.

In certain aspects, the disclosure relates to heteromultimers that comprise at least one Cryptic family protein 1B polypeptide, which includes fragments, functional variants, and modified forms thereof, and at least one Crim1 polypeptide, which includes fragments, functional variants, and modified forms thereof. In some embodiments, the Cryptic family protein 1B:Crim1 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 533 or 534. In some embodiments, the Cryptic family protein 1B:Crim1 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-30 of SEQ ID NO: 533 and ends at any one of amino acids 82-223 of SEQ ID NO: 533. In some embodiments, the Cryptic family protein 1B:Crim1 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 537 or 538. In some embodiments, the Cryptic family protein 1B:Crim1 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 35-37 of SEQ ID NO: 537 and ends at any one of amino acids 873-939 of SEQ ID NO: 537. In certain preferred embodiments, Cryptic family protein 1B:Crim1 heteromultimers are soluble. In some embodiments, a Cryptic family protein 1B:Crim1 heteromultimer of the disclosure binds to one or more TGF-beta superfamily ligands (e.g., binds to one or more TGF-beta superfamily ligands with a K_{D} of at least 1×10⁻⁷). In some embodiments, a Cryptic family protein 1B:Crim1 heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a Cryptic family protein 1B:Crim1 heteromultimer of the disclosure has a different TGF-beta ligand binding and/or inhibition profile (specificity) compared to a corresponding homomultimer (e.g., Cryptic family protein 1B and Crim1 homomultimers). In some embodiments, a Cryptic family protein 1B:Crim1 heteromultimer of the disclosure is a heterodimer.

In certain aspects, the disclosure relates to heteromultimers that comprise at least one Cryptic family protein 1B polypeptide, which includes fragments, functional variants, and modified forms thereof, and at least one Crim2 polypeptide, which includes fragments, functional variants, and modified forms thereof. In some embodiments, the Cryptic family protein 1B:Crim2 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 533 or 534. In some embodiments, the Cryptic family protein 1B:Crim2 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-30 of SEQ ID NO: 533 and ends at any one of amino acids 82-223 of SEQ ID NO: 533. In some embodiments, the Cryptic family protein 1B:Crim2 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 541, 542, 545, or 546. In some embodiments, the Cryptic family protein 1B:Crim2 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-138 of SEQ ID NO: 541 and ends at any one of amino acids 1298-1503 of SEQ ID NO: 541. In some embodiments, the Cryptic family protein 1B:Crim2 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 24-138 of SEQ ID NO: 545 and ends at any one of amino acids 539-814 of SEQ ID NO: 545. In certain preferred embodiments, Cryptic family protein 1B:Crim2 heteromultimers are soluble. In some embodiments, a Cryptic family protein 1B:Crim2 heteromultimer of the disclosure binds to one or more TGF-beta superfamily ligands (e.g., binds to one or more TGF-beta superfamily ligands with a K_{D} of at least 1×10⁻⁷). In some embodiments, a Cryptic family protein 1B:Crim2 heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a Cryptic family protein 1B:Crim2 heteromultimer of the disclosure has a different TGF-beta ligand binding and/or inhibition profile (specificity) compared to a corresponding homomultimer (e.g., Cryptic family protein 1B and Crim2 homomultimers). In some embodiments, a Cryptic family protein 1B:Crim2 heteromultimer of the disclosure is a heterodimer.

In certain aspects, the disclosure relates to heteromultimers that comprise at least one Cryptic family protein 1B polypeptide, which includes fragments, functional variants, and modified forms thereof, and at least one BAMBI polypeptide, which includes fragments, functional variants, and modified forms thereof. In some embodiments, the Cryptic family protein 1B:BAMBI heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 533 or 534. In some embodiments, the Cryptic family protein 1B:BAMBI heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-30 of SEQ ID NO: 533 and ends at any one of amino acids 82-223 of SEQ ID NO: 533. In some embodiments, the Cryptic family protein 1B:BAMBI heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 549 or 550. In some embodiments, the Cryptic family protein 1B:BAMBI heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 21-30 of SEQ ID NO: 549 and ends at any one of amino acids 104-152 of SEQ ID NO: 549. In certain preferred embodiments, Cryptic family protein 1B:BAMBI heteromultimers are soluble. In some embodiments, a Cryptic family protein 1B:BAMBI heteromultimer of the disclosure binds to one or more TGF-beta superfamily ligands (e.g., binds to one or more TGF-beta superfamily ligands with a K_{D} of at least 1×10⁻⁷). In some embodiments, a Cryptic family protein 1B:BAMBI heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a Cryptic family protein 1B:BAMBI of the disclosure has a different TGF-beta ligand binding and/or inhibition profile (specificity) compared to a corresponding homomultimer (e.g., Cryptic family protein 1B and BAMBI homomultimers). In some embodiments, a Cryptic family protein 1B:BAMBI heteromultimer of the disclosure is a heterodimer.

In certain aspects, the disclosure relates to heteromultimers that comprise at least one Cryptic family protein 1B polypeptide, which includes fragments, functional variants, and modified forms thereof, and at least one BMPER polypeptide, which includes fragments, functional variants, and modified forms thereof. In some embodiments, the Cryptic family protein 1B:BMPER heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 533 or 534. In some embodiments, the Cryptic family protein 1B:BMPER heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-30 of SEQ ID NO: 533 and ends at any one of amino acids 82-223 of SEQ ID NO: 533. In some embodiments, the Cryptic family protein 1B:BMPER heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 553 or 554. In some embodiments, the Cryptic family protein 1B:BMPER heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 39-50 of SEQ ID NO: 553 and ends at any one of amino acids 364-369 of SEQ ID NO: 553. In some embodiments, the Cryptic family protein 1B:BMPER heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 370-386 of SEQ ID NO: 553 and ends at any one of amino acids 682-685 of SEQ ID NO: 553. In some embodiments, the Cryptic family protein 1B:BMPER heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 39-50 of SEQ ID NO: 553 and ends at any one of amino acids 682-685 of SEQ ID NO: 553. In some embodiments, the Cryptic family protein 1B:BMPER heteromultimer comprises a BMPER protein, wherein the BMPER protein is a dimer comprising a first polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 39-50 of SEQ ID NO: 553 and ends at any one of amino acids 364-369 of SEQ ID NO: 553, and second polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 370-386 of SEQ ID NO: 553, and ends at any one of amino acids 682-685 of SEQ ID NO: 553. In some embodiments, the Cryptic family protein 1B:BMPER heteromultimer comprises a single chain ligand trap that comprises a first BMPER polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 39-50 of SEQ ID NO: 553 and ends at any one of amino acids 364-369 of SEQ ID NO: 553, and second BMPER polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 370-386 of SEQ ID NO: 553 and ends at any one of amino acids 682-685 of SEQ ID NO: 553. In certain preferred embodiments, Cryptic family protein 1B:BMPER heteromultimers are soluble. In some embodiments, a Cryptic family protein 1B:BMPER heteromultimer of the disclosure binds to one or more TGF-beta superfamily ligands (e.g., binds to one or more TGF-beta superfamily ligands with a K_{D} of at least 1×10⁻⁷). In some embodiments, a Cryptic family protein 1B:BMPER heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a Cryptic family protein 1B:BMPER heteromultimer of the disclosure has a different TGF-beta ligand binding and/or inhibition profile (specificity) compared to a corresponding homomultimer (e.g., Cryptic family protein 1B and BMPER homomultimers). In some embodiments, a Cryptic family protein 1B:BMPER heteromultimer of the disclosure is a heterodimer.

In certain aspects, the disclosure relates to heteromultimers that comprise at least one Cryptic family protein 1B polypeptide, which includes fragments, functional variants, and modified forms thereof, and at least one RGM-A polypeptide, which includes fragments, functional variants, and modified forms thereof. In some embodiments, the Cryptic family protein 1B:RGM-A heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 533 or 534. In some embodiments, the Cryptic family protein 1B:RGM-A heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-30 of SEQ ID NO: 533 and ends at any one of amino acids 82-223 of SEQ ID NO: 533. In some embodiments, the Cryptic family protein 1B:RGM-A heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 553 or 554. In some embodiments, the Cryptic family protein 1B:RGM-A heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-177 of SEQ ID NO: 561 and ends at any one of amino acids 430-458 of SEQ ID NO: 561. In some embodiments, the Cryptic family protein 1B:RGM-A heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-153 of SEQ ID NO: 565 and ends at any one of amino acids 406-434 of SEQ ID NO: 565. In some embodiments, the Cryptic family protein 1B:RGM-A heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-169 of SEQ ID NO: 569 and ends at any one of amino acids 422-450 of SEQ ID NO: 569. In certain preferred embodiments, Cryptic family protein 1B:RGM-A heteromultimers are soluble. In some embodiments, a Cryptic family protein 1B:RGM-A heteromultimer of the disclosure binds to one or more TGF-beta superfamily ligands (e.g., binds to one or more TGF-beta superfamily ligands with a K_{D} of at least 1×10⁻⁷). In some embodiments, a Cryptic family protein 1B:RGM-A heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a Cryptic family protein 1B:RGM-A heteromultimer of the disclosure has a different TGF-beta ligand binding and/or inhibition profile (specificity) compared to a corresponding homomultimer (e.g., Cryptic family protein 1B and RGM-A homomultimers). In some embodiments, a Cryptic family protein 1B:RGM-A heteromultimer of the disclosure is a heterodimer.

In certain aspects, the disclosure relates to heteromultimers that comprise at least one Cryptic family protein 1B polypeptide, which includes fragments, functional variants, and modified forms thereof, and at least one RGM-B polypeptide, which includes fragments, functional variants, and modified forms thereof. In some embodiments, the Cryptic family protein 1B:RGM-B heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 533 or 534. In some embodiments, the Cryptic family protein 1B:RGM-B heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-30 of SEQ ID NO: 533 and ends at any one of amino acids 82-223 of SEQ ID NO: 533. In some embodiments, the Cryptic family protein 1B:RGM-B heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 557 or 558. In some embodiments, the Cryptic family protein 1B:RGM-B heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-87 of SEQ ID NO: 557 and ends at any one of amino acids 452-478 of SEQ ID NO: 557. In some embodiments, the Cryptic family protein 1B:RGM-B heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 210-222 of SEQ ID NO: 557 and ends at any one of amino acids 413-452 of SEQ ID NO: 557. In some embodiments, the Cryptic family protein 1B:RGM-B heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 87-95 of SEQ ID NO: 557 and ends at any one of amino acids 204-209 of SEQ ID NO: 557. In some embodiments, the Cryptic family protein 1B:RGM-B heteromultimer comprises a RGM-B protein, wherein the RGM-B protein is a dimer comprising a first polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 87-95 of SEQ ID NO: 557 and ends at any one of amino acids 204-209 of SEQ ID NO: 557 and second polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 210-222 of SEQ ID NO: 557 and ends at any one of amino acids 413-452 of SEQ ID NO: 557. In some embodiments, the Cryptic family protein 1B:RGM-B heteromultimer comprises a single chain ligand trap that comprises a first RGM-B polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 87-95 of SEQ ID NO: 557 and ends at any one of amino acids 204-209 of SEQ ID NO: 557 and second RGM-B polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 210-222 of SEQ ID NO: 557 and ends at any one of amino acids 413-452 of SEQ ID NO: 557. In certain preferred embodiments, Cryptic family protein 1B:RGM-B heteromultimers are soluble. In some embodiments, a Cryptic family protein 1B:RGM-B heteromultimer of the disclosure binds to one or more TGF-beta superfamily ligands (e.g., binds to one or more TGF-beta superfamily ligands with a K_{D} of at least 1×10⁻⁷). In some embodiments, a Cryptic family protein 1B:RGM-B heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a Cryptic family protein 1B:RGM-B heteromultimer of the disclosure has a different TGF-beta ligand binding and/or inhibition profile (specificity) compared to a corresponding homomultimer (e.g., Cryptic family protein 1B and RGM-B homomultimers). In some embodiments, a Cryptic family protein 1B:RGM-B of the disclosure is a heterodimer.

In certain aspects, the disclosure relates to heteromultimers that comprise at least one Cryptic family protein 1B polypeptide, which includes fragments, functional variants, and modified forms thereof, and at least one hemojuvelin polypeptide, which includes fragments, functional variants, and modified forms thereof. In some embodiments, the Cryptic family protein 1B:hemojuvelin heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 533 or 534. In some embodiments, the Cryptic family protein 1B:hemojuvelin heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-30 of SEQ ID NO: 533 and ends at any one of amino acids 82-223 of SEQ ID NO: 533. In some embodiments, the Cryptic family protein 1B:hemojuvelin heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 573, 574, 577, 578, 581, or 582. In some embodiments, the Cryptic family protein 1B:hemojuvelin heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-36 of SEQ ID NO: 573 and ends at any one of amino acids 400-426 of SEQ ID NO: 573. In some embodiments, the Cryptic family protein 1B:hemojuvelin heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 36-42 of SEQ ID NO: 573 and ends at any one of amino acids 167-172 of SEQ ID NO: 573. In some embodiments, the Cryptic family protein 1B:hemojuvelin heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 173-185 of SEQ ID NO: 573 and ends at any one of amino acids 361-400 of SEQ ID NO: 573. In some embodiments, the Cryptic family protein 1B:hemojuvelin heteromultimer comprises a hemojuvelin protein that is a dimer comprising a first polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 36-42 of SEQ ID NO: 573, and ends at any one of amino acids 167-172 of SEQ ID NO: 573 and second polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 173-185 of SEQ ID NO: 573 and ends at any one of amino acids 361-400 of SEQ ID NO: 573. In some embodiments, the Cryptic family protein 1B:hemojuvelin heteromultimer comprises a single chain ligand trap that comprises a first hemojuvelin polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 36-42 of SEQ ID NO: 573 and ends at any one of amino acids 167-172 of SEQ ID NO: 573 and second hemojuvelin polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 173-185 of SEQ ID NO: 573 and ends at any one of amino acids 361-400 of SEQ ID NO: 573. In some embodiments, the Cryptic family protein 1B:hemojuvelin heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-6 of SEQ ID NO: 577 and ends at any one of amino acids 287-313 of SEQ ID NO: 577. In some embodiments, the Cryptic family protein 1B:hemojuvelin heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-6 of SEQ ID NO: 577 and ends at any one of amino acids 54-59 of SEQ ID NO: 577. In some embodiments, the Cryptic family protein 1B:hemojuvelin heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 60-72 of SEQ ID NO: 577 and ends at any one of amino acids 248-287 of SEQ ID NO: 577. In some embodiments, the Cryptic family protein 1B:hemojuvelin heteromultimer comprises a hemojuvelin protein, wherein the hemojuvelin protein is a dimer comprising a first polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 1-6 of SEQ ID NO: 577 and ends at any one of amino acids 54-59 of SEQ ID NO: 577, and second polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 60-72 of SEQ ID NO: 577 and ends at any one of amino acids 248-287 of SEQ ID NO: 577. In some embodiments, the Cryptic family protein 1B:hemojuvelin heteromultimer comprises a single chain ligand trap that comprises a first hemojuvelin polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 1-6 of SEQ ID NO: 577 and ends at any one of amino acids 54-59 of SEQ ID NO: 577, and second hemojuvelin polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 60-72 of SEQ ID NO: 577, and ends at any one of amino acids 248-287 of SEQ ID NO: 577. In some embodiments, the Cryptic family protein 1B:hemojuvelin heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-4 of SEQ ID NO: 581 and ends at any one of amino acids 135-200 of SEQ ID NO: 581. In certain preferred embodiments, Cryptic family protein 1B:hemojuvelin heteromultimers are soluble. In some embodiments, a Cryptic family protein 1B:hemojuvelin heteromultimer of the disclosure binds to one or more TGF-beta superfamily ligands (e.g., binds to one or more TGF-beta superfamily ligands with a K_{D} of at least 1×10⁻⁷). In some embodiments, a Cryptic family protein 1B:hemojuvelin heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a Cryptic family protein 1B:hemojuvelin heteromultimer of the disclosure has a different TGF-beta ligand binding and/or inhibition profile (specificity) compared to a corresponding homomultimer (e.g., Cryptic family protein 1B and hemojuvelin homomultimers). In some embodiments, a Cryptic family protein 1B:hemojuvelin heteromultimer of the disclosure is a heterodimer.

In certain aspects, the disclosure relates to heteromultimers that comprise at least one Crim1 polypeptide, which includes fragments, functional variants, and modified forms thereof, and at least one Crim2 polypeptide, which includes fragments, functional variants, and modified forms thereof. In some embodiments, the Crim1:Crim2 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 537 or 538. In some embodiments, the Crim1:Crim2 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 35-37 of SEQ ID NO: 537 and ends at any one of amino acids 873-939 of SEQ ID NO: 537. In some embodiments, the Crim1:Crim2 heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 541, 542, 545, or 546. In some embodiments, the Crim1:Crim2 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-138 of SEQ ID NO: 541 and ends at any one of amino acids 1298-1503 of SEQ ID NO: 541. In some embodiments, the Crim1:Crim2 heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 24-138 of SEQ ID NO: 545 and ends at any one of amino acids 539-814 of SEQ ID NO: 545. In certain preferred embodiments, Crim1:Crim2 heteromultimers are soluble. In some embodiments, a Crim1:Crim2 heteromultimer of the disclosure binds to one or more TGF-beta superfamily ligands (e.g., binds to one or more TGF-beta superfamily ligands with a K_{D} of at least 1×10⁻⁷). In some embodiments, a Crim1:Crim2 heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a Crim1:Crim2 heteromultimer of the disclosure has a different TGF-beta ligand binding and/or inhibition profile (specificity) compared to a corresponding homomultimer (e.g., Crim1 and Crim2 homomultimers). In some embodiments, a Crim1:Crim2 heteromultimer of the disclosure is a heterodimer.

In certain aspects, the disclosure relates to heteromultimers that comprise at least one Crim1 polypeptide, which includes fragments, functional variants, and modified forms thereof, and at least one BAMBI polypeptide, which includes fragments, functional variants, and modified forms thereof.

In some embodiments, the Crim1:BAMBI heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 537 or 538. In some embodiments, the Crim1:BAMBI heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 35-37 of SEQ ID NO: 537 and ends at any one of amino acids 873-939 of SEQ ID NO: 537. In some embodiments, the Crim1:BAMBI heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 549 or 550. In some embodiments, the Crim1:BAMBI heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 21-30 of SEQ ID NO: 549 and ends at any one of amino acids 104-152 of SEQ ID NO: 549. In certain preferred embodiments, Crim1:BAMBI heteromultimers are soluble. In some embodiments, a Crim1:BAMBI heteromultimer of the disclosure binds to one or more TGF-beta superfamily ligands (e.g., binds to one or more TGF-beta superfamily ligands with a K_{D} of at least 1×10⁻⁷). In some embodiments, a Crim1:BAMBI heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a Crim1:BAMBI heteromultimer of the disclosure has a different TGF-beta ligand binding and/or inhibition profile (specificity) compared to a corresponding homomultimer (e.g., Crim1 and BAMBI homomultimers). In some embodiments, a Crim1:BAMBI heteromultimer of the disclosure is a heterodimer.

In certain aspects, the disclosure relates to heteromultimers that comprise at least one Crim1 polypeptide, which includes fragments, functional variants, and modified forms thereof, and at least one BMPER polypeptide, which includes fragments, functional variants, and modified forms thereof. In some embodiments, the Crim1:BMPER heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 537 or 538. In some embodiments, the Crim1:BMPER heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 35-37 of SEQ ID NO: 537 and ends at any one of amino acids 873-939 of SEQ ID NO: 537. In some embodiments, the Crim1:BMPER heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 553 or 554. In some embodiments, the Crim1:BMPER heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 39-50 of SEQ ID NO: 553 and ends at any one of amino acids 364-369 of SEQ ID NO: 553. In some embodiments, the Crim1:BMPER heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 370-386 of SEQ ID NO: 553 and ends at any one of amino acids 682-685 of SEQ ID NO: 553. In some embodiments, the Crim1:BMPER heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 39-50 of SEQ ID NO: 553 and ends at any one of amino acids 682-685 of SEQ ID NO: 553. In some embodiments, the Crim1:BMPER heteromultimer comprises a BMPER protein, wherein the BMPER protein is a dimer comprising a first polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 39-50 of SEQ ID NO: 553 and ends at any one of amino acids 364-369 of SEQ ID NO: 553, and second polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 370-386 of SEQ ID NO: 553, and ends at any one of amino acids 682-685 of SEQ ID NO: 553. In some embodiments, the Crim1:BMPER heteromultimer comprises a single chain ligand trap that comprises a first BMPER polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 39-50 of SEQ ID NO: 553 and ends at any one of amino acids 364-369 of SEQ ID NO: 553, and second BMPER polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 370-386 of SEQ ID NO: 553 and ends at any one of amino acids 682-685 of SEQ ID NO: 553. In certain preferred embodiments, Crim1:BMPER heteromultimers are soluble. In some embodiments, a Crim1:BMPER heteromultimer of the disclosure binds to one or more TGF-beta superfamily ligands (e.g., binds to one or more TGF-beta superfamily ligands with a K_{D} of at least 1×10⁻⁷). In some embodiments, a Crim1:BMPER heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a Crim1:BMPER heteromultimer of the disclosure has a different TGF-beta ligand binding and/or inhibition profile (specificity) compared to a corresponding homomultimer (e.g., Crim1 and BMPER homomultimers). In some embodiments, a Crim1:BMPER heteromultimer of the disclosure is a heterodimer.

In certain aspects, the disclosure relates to heteromultimers that comprise at least one Crim1 polypeptide, which includes fragments, functional variants, and modified forms thereof, and at least one RGM-A polypeptide, which includes fragments, functional variants, and modified forms thereof. In some embodiments, the Crim1:RGM-A heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 537 or 538. In some embodiments, the Crim1:RGM-A heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 35-37 of SEQ ID NO: 537 and ends at any one of amino acids 873-939 of SEQ ID NO: 537. In some embodiments, the Crim1:RGM-A heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 553 or 554. In some embodiments, the Crim1:RGM-A heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-177 of SEQ ID NO: 561 and ends at any one of amino acids 430-458 of SEQ ID NO: 561. In some embodiments, the Crim1:RGM-A heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-153 of SEQ ID NO: 565 and ends at any one of amino acids 406-434 of SEQ ID NO: 565. In some embodiments, the Crim1:RGM-A heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-169 of SEQ ID NO: 569 and ends at any one of amino acids 422-450 of SEQ ID NO: 569. In certain preferred embodiments, Crim1:RGM-A heteromultimers are soluble. In some embodiments, a Crim1:RGM-A heteromultimer of the disclosure binds to one or more TGF-beta superfamily ligands (e.g., binds to one or more TGF-beta superfamily ligands with a K_{D} of at least 1×10⁻⁷). In some embodiments, a Crim1:RGM-A heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a Crim1:RGM-A heteromultimer of the disclosure has a different TGF-beta ligand binding and/or inhibition profile (specificity) compared to a corresponding homomultimer (e.g., Crim1 and RGM-A homomultimers). In some embodiments, a Crim1:RGM-A heteromultimer of the disclosure is a heterodimer.

In certain aspects, the disclosure relates to heteromultimers that comprise at least one Crim1 polypeptide, which includes fragments, functional variants, and modified forms thereof, and at least one RGM-B polypeptide, which includes fragments, functional variants, and modified forms thereof. In some embodiments, the Crim1:RGM-B heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 537 or 538. In some embodiments, the Crim1:RGM-B heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 35-37 of SEQ ID NO: 537 and ends at any one of amino acids 873-939 of SEQ ID NO: 537. In some embodiments, the Crim1:RGM-B heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 557 or 558. In some embodiments, the Crim1:RGM-B heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-87 of SEQ ID NO: 557 and ends at any one of amino acids 452-478 of SEQ ID NO: 557. In some embodiments, the Crim1:RGM-B heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 210-222 of SEQ ID NO: 557 and ends at any one of amino acids 413-452 of SEQ ID NO: 557. In some embodiments, the Crim1:RGM-B heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 87-95 of SEQ ID NO: 557 and ends at any one of amino acids 204-209 of SEQ ID NO: 557. In some embodiments, the Crim1:RGM-B heteromultimer comprises a RGM-B protein, wherein the RGM-B protein is a dimer comprising a first polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 87-95 of SEQ ID NO: 557 and ends at any one of amino acids 204-209 of SEQ ID NO: 557 and second polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 210-222 of SEQ ID NO: 557 and ends at any one of amino acids 413-452 of SEQ ID NO: 557. In some embodiments, the Crim1:RGM-B heteromultimer comprises a single chain ligand trap that comprises a first RGM-B polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 87-95 of SEQ ID NO: 557 and ends at any one of amino acids 204-209 of SEQ ID NO: 557 and second RGM-B polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 210-222 of SEQ ID NO: 557 and ends at any one of amino acids 413-452 of SEQ ID NO: 557. In certain preferred embodiments, Crim1:RGM-B heteromultimers are soluble. In some embodiments, a Crim1:RGM-B heteromultimer of the disclosure binds to one or more TGF-beta superfamily ligands (e.g., binds to one or more TGF-beta superfamily ligands with a K_{D} of at least 1×10⁻⁷). In some embodiments, a Crim1:RGM-B heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a Crim1:RGM-B heteromultimer of the disclosure has a different TGF-beta ligand binding and/or inhibition profile (specificity) compared to a corresponding homomultimer (e.g., Crim1 and RGM-B homomultimers). In some embodiments, a Crim1:RGM-B heteromultimer of the disclosure is a heterodimer.

In certain aspects, the disclosure relates to heteromultimers that comprise at least one Crim1 polypeptide, which includes fragments, functional variants, and modified forms thereof, and at least one hemojuvelin polypeptide, which includes fragments, functional variants, and modified forms thereof. In some embodiments, the Crim1:hemojuvelin heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 537 or 538. In some embodiments, the Crim1:hemojuvelin heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 35-37 of SEQ ID NO: 537 and ends at any one of amino acids 873-939 of SEQ ID NO: 537. In some embodiments, the Crim1:hemojuvelin heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 573, 574, 577, 578, 581, or 582. In some embodiments, the Crim1:hemojuvelin heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-36 of SEQ ID NO: 573 and ends at any one of amino acids 400-426 of SEQ ID NO: 573. In some embodiments, the Crim1:hemojuvelin heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 36-42 of SEQ ID NO: 573 and ends at any one of amino acids 167-172 of SEQ ID NO: 573. In some embodiments, the Crim1:hemojuvelin heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 173-185 of SEQ ID NO: 573 and ends at any one of amino acids 361-400 of SEQ ID NO: 573. In some embodiments, the Crim1:hemojuvelin heteromultimer comprises a hemojuvelin protein that is a dimer comprising a first polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 36-42 of SEQ ID NO: 573, and ends at any one of amino acids 167-172 of SEQ ID NO: 573 and second polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 173-185 of SEQ ID NO: 573 and ends at any one of amino acids 361-400 of SEQ ID NO: 573. In some embodiments, the Crim1:hemojuvelin heteromultimer comprises a single chain ligand trap that comprises a first hemojuvelin polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 36-42 of SEQ ID NO: 573 and ends at any one of amino acids 167-172 of SEQ ID NO: 573 and second hemojuvelin polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 173-185 of SEQ ID NO: 573 and ends at any one of amino acids 361-400 of SEQ ID NO: 573. In some embodiments, the Crim1:hemojuvelin heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-6 of SEQ ID NO: 577 and ends at any one of amino acids 287-313 of SEQ ID NO: 577. In some embodiments, the Crim1:hemojuvelin heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-6 of SEQ ID NO: 577 and ends at any one of amino acids 54-59 of SEQ ID NO: 577. In some embodiments, the Crim1:hemojuvelin heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 60-72 of SEQ ID NO: 577 and ends at any one of amino acids 248-287 of SEQ ID NO: 577. In some embodiments, the Crim1:hemojuvelin heteromultimer comprises a hemojuvelin protein, wherein the hemojuvelin protein is a dimer comprising a first polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 1-6 of SEQ ID NO: 577 and ends at any one of amino acids 54-59 of SEQ ID NO: 577, and second polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 60-72 of SEQ ID NO: 577 and ends at any one of amino acids 248-287 of SEQ ID NO: 577. In some embodiments, the Crim1:hemojuvelin heteromultimer comprises a single chain ligand trap that comprises a first hemojuvelin polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 1-6 of SEQ ID NO: 577 and ends at any one of amino acids 54-59 of SEQ ID NO: 577, and second hemojuvelin polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 60-72 of SEQ ID NO: 577, and ends at any one of amino acids 248-287 of SEQ ID NO: 577. In some embodiments, the Crim1:hemojuvelin heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-4 of SEQ ID NO: 581 and ends at any one of amino acids 135-200 of SEQ ID NO: 581. In certain preferred embodiments, Crim1:hemojuvelin heteromultimers are soluble. In some embodiments, a Crim1:hemojuvelin heteromultimer of the disclosure binds to one or more TGF-beta superfamily ligands (e.g., binds to one or more TGF-beta superfamily ligands with a K_{D} of at least 1×10⁻⁷). In some embodiments, a Crim1:hemojuvelin heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a Crim1:hemojuvelin heteromultimer of the disclosure has a different TGF-beta ligand binding and/or inhibition profile (specificity) compared to a corresponding homomultimer (e.g., Crim1 and hemojuvelin homomultimers). In some embodiments, a Crim1:hemojuvelin heteromultimer of the disclosure is a heterodimer.

In certain aspects, the disclosure relates to heteromultimers that comprise at least one Crim2 polypeptide, which includes fragments, functional variants, and modified forms thereof, and at least one BAMBI polypeptide, which includes fragments, functional variants, and modified forms thereof. In some embodiments, the Crim2:BAMBI heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 541, 542, 545, or 546. In some embodiments, the Crim2:BAMBI heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-138 of SEQ ID NO: 541 and ends at any one of amino acids 1298-1503 of SEQ ID NO: 541. In some embodiments, the Crim2:BAMBI heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 24-138 of SEQ ID NO: 545 and ends at any one of amino acids 539-814 of SEQ ID NO: 545. In some embodiments, the Crim2:BAMBI heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 549 or 550. In some embodiments, the Crim2:BAMBI heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 21-30 of SEQ ID NO: 549 and ends at any one of amino acids 104-152 of SEQ ID NO: 549. In certain preferred embodiments, Crim2:BAMBI heteromultimers are soluble. In some embodiments, a Crim2:BAMBI heteromultimer of the disclosure binds to one or more TGF-beta superfamily ligands (e.g., binds to one or more TGF-beta superfamily ligands with a K_{D} of at least 1×10⁻⁷). In some embodiments, a Crim2:BAMBI heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a Crim2:BAMBI heteromultimer of the disclosure has a different TGF-beta ligand binding and/or inhibition profile (specificity) compared to a corresponding homomultimer (e.g., Crim2 and BAMBI homomultimers). In some embodiments, a Crim2:BAMBI heteromultimer of the disclosure is a heterodimer.

In certain aspects, the disclosure relates to heteromultimers that comprise at least one Crim2 polypeptide, which includes fragments, functional variants, and modified forms thereof, and at least one BMPER polypeptide, which includes fragments, functional variants, and modified forms thereof. In some embodiments, the Crim2:BMPER heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 541, 542, 545, or 546. In some embodiments, the Crim2:BMPER heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-138 of SEQ ID NO: 541 and ends at any one of amino acids 1298-1503 of SEQ ID NO: 541. In some embodiments, the Crim2:BMPER heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 24-138 of SEQ ID NO: 545 and ends at any one of amino acids 539-814 of SEQ ID NO: 545. In some embodiments, the Crim2:BMPER heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 553 or 554. In some embodiments, the Crim2:BMPER heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 39-50 of SEQ ID NO: 553 and ends at any one of amino acids 364-369 of SEQ ID NO: 553. In some embodiments, the Crim2:BMPER heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 370-386 of SEQ ID NO: 553 and ends at any one of amino acids 682-685 of SEQ ID NO: 553. In some embodiments, the Crim2:BMPER heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 39-50 of SEQ ID NO: 553 and ends at any one of amino acids 682-685 of SEQ ID NO: 553. In some embodiments, the Crim2:BMPER heteromultimer comprises a BMPER protein, wherein the BMPER protein is a dimer comprising a first polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 39-50 of SEQ ID NO: 553 and ends at any one of amino acids 364-369 of SEQ ID NO: 553, and second polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 370-386 of SEQ ID NO: 553, and ends at any one of amino acids 682-685 of SEQ ID NO: 553. In some embodiments, the Crim2:BMPER heteromultimer comprises a single chain ligand trap that comprises a first BMPER polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 39-50 of SEQ ID NO: 553 and ends at any one of amino acids 364-369 of SEQ ID NO: 553, and second BMPER polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 370-386 of SEQ ID NO: 553 and ends at any one of amino acids 682-685 of SEQ ID NO: 553. In certain preferred embodiments, Crim2:BMPER heteromultimers are soluble. In some embodiments, a Crim2:BMPER heteromultimer of the disclosure binds to one or more TGF-beta superfamily ligands (e.g., binds to one or more TGF-beta superfamily ligands with a K_{D} of at least 1×10⁻⁷). In some embodiments, a Crim2:BMPER heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a Crim2:BMPER heteromultimer of the disclosure has a different TGF-beta ligand binding and/or inhibition profile (specificity) compared to a corresponding homomultimer (e.g., Crim2 and BMPER homomultimers). In some embodiments, a Crim2:BMPER heteromultimer of the disclosure is a heterodimer.

In certain aspects, the disclosure relates to heteromultimers that comprise at least one Crim2 polypeptide, which includes fragments, functional variants, and modified forms thereof, and at least one RGM-A polypeptide, which includes fragments, functional variants, and modified forms thereof. In some embodiments, the Crim2:RGM-A heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 541, 542, 545, or 546. In some embodiments, the Crim2:RGM-A heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-138 of SEQ ID NO: 541 and ends at any one of amino acids 1298-1503 of SEQ ID NO: 541. In some embodiments, the Crim2:RGM-A heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 24-138 of SEQ ID NO: 545 and ends at any one of amino acids 539-814 of SEQ ID NO: 545. In some embodiments, the Crim2:RGM-A heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 553 or 554. In some embodiments, the Crim2:RGM-A heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-177 of SEQ ID NO: 561 and ends at any one of amino acids 430-458 of SEQ ID NO: 561. In some embodiments, the Crim2:RGM-A heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-153 of SEQ ID NO: 565 and ends at any one of amino acids 406-434 of SEQ ID NO: 565. In some embodiments, the Crim2:RGM-A heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-169 of SEQ ID NO: 569 and ends at any one of amino acids 422-450 of SEQ ID NO: 569. In certain preferred embodiments, Crim2:RGM-A heteromultimers are soluble. In some embodiments, a Crim2:RGM-A heteromultimer of the disclosure binds to one or more TGF-beta superfamily ligands (e.g., binds to one or more TGF-beta superfamily ligands with a K_{D} of at least 1×10⁻⁷). In some embodiments, a Crim2:RGM-A heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a Crim2:RGM-A heteromultimer of the disclosure has a different TGF-beta ligand binding and/or inhibition profile (specificity) compared to a corresponding homomultimer (e.g., Crim2 and RGM-A homomultimers). In some embodiments, a Crim2:RGM-A heteromultimer of the disclosure is a heterodimer.

In certain aspects, the disclosure relates to heteromultimers that comprise at least one Crim2 polypeptide, which includes fragments, functional variants, and modified forms thereof, and at least one RGM-B polypeptide, which includes fragments, functional variants, and modified forms thereof. In some embodiments, the Crim2:RGM-B heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 541, 542, 545, or 546. In some embodiments, the Crim2:RGM-B heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-138 of SEQ ID NO: 541 and ends at any one of amino acids 1298-1503 of SEQ ID NO: 541. In some embodiments, the Crim2:RGM-B heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 24-138 of SEQ ID NO: 545 and ends at any one of amino acids 539-814 of SEQ ID NO: 545. In some embodiments, the Crim2:RGM-B heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 557 or 558. In some embodiments, the Crim2:RGM-B heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-87 of SEQ ID NO: 557 and ends at any one of amino acids 452-478 of SEQ ID NO: 557. In some embodiments, the Crim2:RGM-B heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 210-222 of SEQ ID NO: 557 and ends at any one of amino acids 413-452 of SEQ ID NO: 557. In some embodiments, the Crim2:RGM-B heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 87-95 of SEQ ID NO: 557 and ends at any one of amino acids 204-209 of SEQ ID NO: 557. In some embodiments, the Crim2:RGM-B heteromultimer comprises a RGM-B protein, wherein the RGM-B protein is a dimer comprising a first polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 87-95 of SEQ ID NO: 557 and ends at any one of amino acids 204-209 of SEQ ID NO: 557 and second polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 210-222 of SEQ ID NO: 557 and ends at any one of amino acids 413-452 of SEQ ID NO: 557. In some embodiments, the Crim2:RGM-B heteromultimer comprises a single chain ligand trap that comprises a first RGM-B polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 87-95 of SEQ ID NO: 557 and ends at any one of amino acids 204-209 of SEQ ID NO: 557 and second RGM-B polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 210-222 of SEQ ID NO: 557 and ends at any one of amino acids 413-452 of SEQ ID NO: 557. In certain preferred embodiments, Crim2:RGM-B heteromultimers are soluble. In some embodiments, a Crim2:RGM-B heteromultimer of the disclosure binds to one or more TGF-beta superfamily ligands (e.g., binds to one or more TGF-beta superfamily ligands with a K_{D} of at least 1×10⁻⁷). In some embodiments, a Crim2:RGM-B heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a Crim2:RGM-B heteromultimer of the disclosure has a different TGF-beta ligand binding and/or inhibition profile (specificity) compared to a corresponding homomultimer (e.g., Crim2 and RGM-B homomultimers). In some embodiments, a Crim2:RGM-B heteromultimer of the disclosure is a heterodimer.

In certain aspects, the disclosure relates to heteromultimers that comprise at least one Crim2 polypeptide, which includes fragments, functional variants, and modified forms thereof, and at least one hemojuvelin polypeptide, which includes fragments, functional variants, and modified forms thereof. In some embodiments, the Crim2:hemojuvelin heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 541, 542, 545, or 546. In some embodiments, the Crim2:hemojuvelin heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 26-138 of SEQ ID NO: 541 and ends at any one of amino acids 1298-1503 of SEQ ID NO: 541. In some embodiments, the Crim2:hemojuvelin heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 24-138 of SEQ ID NO: 545 and ends at any one of amino acids 539-814 of SEQ ID NO: 545. In some embodiments, the Crim2:hemojuvelin heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 573, 574, 577, 578, 581, or 582. In some embodiments, the Crim2:hemojuvelin heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-36 of SEQ ID NO: 573 and ends at any one of amino acids 400-426 of SEQ ID NO: 573. In some embodiments, the Crim2:hemojuvelin heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 36-42 of SEQ ID NO: 573 and ends at any one of amino acids 167-172 of SEQ ID NO: 573. In some embodiments, the Crim2:hemojuvelin heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 173-185 of SEQ ID NO: 573 and ends at any one of amino acids 361-400 of SEQ ID NO: 573. In some embodiments, the Crim2:hemojuvelin heteromultimer comprises a hemojuvelin protein that is a dimer comprising a first polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 36-42 of SEQ ID NO: 573, and ends at any one of amino acids 167-172 of SEQ ID NO: 573 and second polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 173-185 of SEQ ID NO: 573 and ends at any one of amino acids 361-400 of SEQ ID NO: 573. In some embodiments, the Crim2:hemojuvelin heteromultimer comprises a single chain ligand trap that comprises a first hemojuvelin polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 36-42 of SEQ ID NO: 573 and ends at any one of amino acids 167-172 of SEQ ID NO: 573 and second hemojuvelin polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 173-185 of SEQ ID NO: 573 and ends at any one of amino acids 361-400 of SEQ ID NO: 573. In some embodiments, the Crim2:hemojuvelin heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-6 of SEQ ID NO: 577 and ends at any one of amino acids 287-313 of SEQ ID NO: 577. In some embodiments, the Crim2:hemojuvelin heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-6 of SEQ ID NO: 577 and ends at any one of amino acids 54-59 of SEQ ID NO: 577. In some embodiments, the Crim2:hemojuvelin heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 60-72 of SEQ ID NO: 577 and ends at any one of amino acids 248-287 of SEQ ID NO: 577. In some embodiments, the Crim2:hemojuvelin heteromultimer comprises a hemojuvelin protein, wherein the hemojuvelin protein is a dimer comprising a first polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 1-6 of SEQ ID NO: 577 and ends at any one of amino acids 54-59 of SEQ ID NO: 577, and second polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 60-72 of SEQ ID NO: 577 and ends at any one of amino acids 248-287 of SEQ ID NO: 577. In some embodiments, the Crim2:hemojuvelin heteromultimer comprises a single chain ligand trap that comprises a first hemojuvelin polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 1-6 of SEQ ID NO: 577 and ends at any one of amino acids 54-59 of SEQ ID NO: 577, and second hemojuvelin polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 60-72 of SEQ ID NO: 577, and ends at any one of amino acids 248-287 of SEQ ID NO: 577. In some embodiments, the Crim2:hemojuvelin heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-4 of SEQ ID NO: 581 and ends at any one of amino acids 135-200 of SEQ ID NO: 581. In certain preferred embodiments, Crim2:hemojuvelin heteromultimers are soluble. In some embodiments, a Crim2:hemojuvelin heteromultimer of the disclosure binds to one or more TGF-beta superfamily ligands (e.g., binds to one or more TGF-beta superfamily ligands with a K_{D} of at least 1×10⁻⁷). In some embodiments, a Crim2:hemojuvelin heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a Crim2:hemojuvelin heteromultimer of the disclosure has a different TGF-beta ligand binding and/or inhibition profile (specificity) compared to a corresponding homomultimer (e.g., Crim2 and hemojuvelin homomultimers). In some embodiments, a Crim2:hemojuvelin heteromultimer of the disclosure is a heterodimer.

In certain aspects, the disclosure relates to heteromultimers that comprise at least one BAMBI polypeptide, which includes fragments, functional variants, and modified forms thereof, and at least one BMPER polypeptide, which includes fragments, functional variants, and modified forms thereof. In some embodiments, the BAMBI:BMPER heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 549 or 550. In some embodiments, the BAMBI:BMPER heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 21-30 of SEQ ID NO: 549 and ends at any one of amino acids 104-152 of SEQ ID NO: 549. In some embodiments, the BAMBI:BMPER heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 553 or 554. In some embodiments, the BAMBI:BMPER heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 39-50 of SEQ ID NO: 553 and ends at any one of amino acids 364-369 of SEQ ID NO: 553. In some embodiments, the BAMBI:BMPER heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 370-386 of SEQ ID NO: 553 and ends at any one of amino acids 682-685 of SEQ ID NO: 553. In some embodiments, the BAMBI:BMPER heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 39-50 of SEQ ID NO: 553 and ends at any one of amino acids 682-685 of SEQ ID NO: 553. In some embodiments, the BAMBI:BMPER heteromultimer comprises a BMPER protein, wherein the BMPER protein is a dimer comprising a first polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 39-50 of SEQ ID NO: 553 and ends at any one of amino acids 364-369 of SEQ ID NO: 553, and second polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 370-386 of SEQ ID NO: 553, and ends at any one of amino acids 682-685 of SEQ ID NO: 553. In some embodiments, the BAMBI:BMPER heteromultimer comprises a single chain ligand trap that comprises a first BMPER polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 39-50 of SEQ ID NO: 553 and ends at any one of amino acids 364-369 of SEQ ID NO: 553, and second BMPER polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 370-386 of SEQ ID NO: 553 and ends at any one of amino acids 682-685 of SEQ ID NO: 553. In certain preferred embodiments, BAMBI:BMPER heteromultimers are soluble. In some embodiments, a BAMBI:BMPER heteromultimer of the disclosure binds to one or more TGF-beta superfamily ligands (e.g., binds to one or more TGF-beta superfamily ligands with a K_{D} of at least 1×10⁻⁷). In some embodiments, a BAMBI:BMPER heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a BAMBI:BMPER heteromultimer of the disclosure has a different TGF-beta ligand binding and/or inhibition profile (specificity) compared to a corresponding homomultimer (e.g., BAMBI and BMPER homomultimers). In some embodiments, a BAMBI:BMPER heteromultimer of the disclosure is a heterodimer.

In certain aspects, the disclosure relates to heteromultimers that comprise at least one BAMBI polypeptide, which includes fragments, functional variants, and modified forms thereof, and at least one RGM-A polypeptide, which includes fragments, functional variants, and modified forms thereof. In some embodiments, the BAMBI:RGM-A heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 549 or 550. In some embodiments, the BAMBI:RGM-A heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 21-30 of SEQ ID NO: 549 and ends at any one of amino acids 104-152 of SEQ ID NO: 549. In some embodiments, the BAMBI:RGM-A heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 553 or 554. In some embodiments, the BAMBI:RGM-A heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-177 of SEQ ID NO: 561 and ends at any one of amino acids 430-458 of SEQ ID NO: 561. In some embodiments, the BAMBI:RGM-A heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-153 of SEQ ID NO: 565 and ends at any one of amino acids 406-434 of SEQ ID NO: 565. In some embodiments, the BAMBI:RGM-A heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-169 of SEQ ID NO: 569 and ends at any one of amino acids 422-450 of SEQ ID NO: 569. In certain preferred embodiments, BAMBI:RGM-A heteromultimers are soluble. In some embodiments, a BAMBI:RGM-A heteromultimer of the disclosure binds to one or more TGF-beta superfamily ligands (e.g., binds to one or more TGF-beta superfamily ligands with a K_{D} of at least 1×10⁻⁷). In some embodiments, a BAMBI:RGM-A heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a BAMBI:RGM-A heteromultimer of the disclosure has a different TGF-beta ligand binding and/or inhibition profile (specificity) compared to a corresponding homomultimer (e.g., BAMBI and RGM-A homomultimers). In some embodiments, a BAMBI:RGM-A heteromultimer of the disclosure is a heterodimer.

In certain aspects, the disclosure relates to heteromultimers that comprise at least one BAMBI polypeptide, which includes fragments, functional variants, and modified forms thereof, and at least one RGM-B polypeptide, which includes fragments, functional variants, and modified forms thereof. In some embodiments, the BAMBI:RGM-B heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 549 or 550. In some embodiments, the BAMBI:RGM-B heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 21-30 of SEQ ID NO: 549 and ends at any one of amino acids 104-152 of SEQ ID NO: 549. In some embodiments, the BAMBI:RGM-B heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 557 or 558. In some embodiments, the BAMBI:RGM-B heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-87 of SEQ ID NO: 557 and ends at any one of amino acids 452-478 of SEQ ID NO: 557. In some embodiments, the BAMBI:RGM-B heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 210-222 of SEQ ID NO: 557 and ends at any one of amino acids 413-452 of SEQ ID NO: 557. In some embodiments, the BAMBI:RGM-B heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 87-95 of SEQ ID NO: 557 and ends at any one of amino acids 204-209 of SEQ ID NO: 557. In some embodiments, the BAMBI:RGM-B heteromultimer comprises a RGM-B protein, wherein the RGM-B protein is a dimer comprising a first polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 87-95 of SEQ ID NO: 557 and ends at any one of amino acids 204-209 of SEQ ID NO: 557 and second polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 210-222 of SEQ ID NO: 557 and ends at any one of amino acids 413-452 of SEQ ID NO: 557. In some embodiments, the BAMBI:RGM-B heteromultimer comprises a single chain ligand trap that comprises a first RGM-B polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 87-95 of SEQ ID NO: 557 and ends at any one of amino acids 204-209 of SEQ ID NO: 557 and second RGM-B polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 210-222 of SEQ ID NO: 557 and ends at any one of amino acids 413-452 of SEQ ID NO: 557. In certain preferred embodiments, BAMBI:RGM-B heteromultimers are soluble. In some embodiments, a BAMBI:RGM-B heteromultimer of the disclosure binds to one or more TGF-beta superfamily ligands (e.g., binds to one or more TGF-beta superfamily ligands with a K_{D} of at least 1×10⁻⁷). In some embodiments, a BAMBI:RGM-B heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a BAMBI:RGM-B heteromultimer of the disclosure has a different TGF-beta ligand binding and/or inhibition profile (specificity) compared to a corresponding homomultimer (e.g., BAMBI and RGM-B homomultimers). In some embodiments, a BAMBI:RGM-B heteromultimer of the disclosure is a heterodimer.

In certain aspects, the disclosure relates to heteromultimers that comprise at least one BAMBI polypeptide, which includes fragments, functional variants, and modified forms thereof, and at least one hemojuvelin polypeptide, which includes fragments, functional variants, and modified forms thereof. In some embodiments, the BAMBI:hemojuvelin heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 549 or 550. In some embodiments, the BAMBI:hemojuvelin heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 21-30 of SEQ ID NO: 549 and ends at any one of amino acids 104-152 of SEQ ID NO: 549. In some embodiments, the BAMBI:hemojuvelin heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 573, 574, 577, 578, 581, or 582. In some embodiments, the BAMBI:hemojuvelin heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-36 of SEQ ID NO: 573 and ends at any one of amino acids 400-426 of SEQ ID NO: 573. In some embodiments, the BAMBI:hemojuvelin heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 36-42 of SEQ ID NO: 573 and ends at any one of amino acids 167-172 of SEQ ID NO: 573. In some embodiments, the BAMBI:hemojuvelin heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 173-185 of SEQ ID NO: 573 and ends at any one of amino acids 361-400 of SEQ ID NO: 573. In some embodiments, the BAMBI:hemojuvelin heteromultimer comprises a hemojuvelin protein that is a dimer comprising a first polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 36-42 of SEQ ID NO: 573, and ends at any one of amino acids 167-172 of SEQ ID NO: 573 and second polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 173-185 of SEQ ID NO: 573 and ends at any one of amino acids 361-400 of SEQ ID NO: 573. In some embodiments, the BAMBI:hemojuvelin heteromultimer comprises a single chain ligand trap that comprises a first hemojuvelin polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 36-42 of SEQ ID NO: 573 and ends at any one of amino acids 167-172 of SEQ ID NO: 573 and second hemojuvelin polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 173-185 of SEQ ID NO: 573 and ends at any one of amino acids 361-400 of SEQ ID NO: 573. In some embodiments, the BAMBI:hemojuvelin heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-6 of SEQ ID NO: 577 and ends at any one of amino acids 287-313 of SEQ ID NO: 577. In some embodiments, the BAMBI:hemojuvelin heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-6 of SEQ ID NO: 577 and ends at any one of amino acids 54-59 of SEQ ID NO: 577. In some embodiments, the BAMBI:hemojuvelin heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 60-72 of SEQ ID NO: 577 and ends at any one of amino acids 248-287 of SEQ ID NO: 577. In some embodiments, the BAMBI:hemojuvelin heteromultimer comprises a hemojuvelin protein, wherein the hemojuvelin protein is a dimer comprising a first polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 1-6 of SEQ ID NO: 577 and ends at any one of amino acids 54-59 of SEQ ID NO: 577, and second polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 60-72 of SEQ ID NO: 577 and ends at any one of amino acids 248-287 of SEQ ID NO: 577. In some embodiments, the BAMBI:hemojuvelin heteromultimer comprises a single chain ligand trap that comprises a first hemojuvelin polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 1-6 of SEQ ID NO: 577 and ends at any one of amino acids 54-59 of SEQ ID NO: 577, and second hemojuvelin polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 60-72 of SEQ ID NO: 577, and ends at any one of amino acids 248-287 of SEQ ID NO: 577. In some embodiments, the BAMBI:hemojuvelin heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-4 of SEQ ID NO: 581 and ends at any one of amino acids 135-200 of SEQ ID NO: 581. In certain preferred embodiments, BAMBI:hemojuvelin heteromultimers are soluble. In some embodiments, a BAMBI:hemojuvelin heteromultimer of the disclosure binds to one or more TGF-beta superfamily ligands (e.g., binds to one or more TGF-beta superfamily ligands with a K_{D} of at least 1×10⁻⁷). In some embodiments, a BAMBI:hemojuvelin heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a BAMBI:hemojuvelin heteromultimer of the disclosure has a different TGF-beta ligand binding and/or inhibition profile (specificity) compared to a corresponding homomultimer (e.g., BAMBI and hemojuvelin homomultimers). In some embodiments, a BAMBI:hemojuvelin heteromultimer of the disclosure is a heterodimer.

In certain aspects, the disclosure relates to heteromultimers that comprise at least one BMPER polypeptide, which includes fragments, functional variants, and modified forms thereof, and at least one RGM-A polypeptide, which includes fragments, functional variants, and modified forms thereof. In some embodiments, the BMPER:RGM-A heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 553 or 554. In some embodiments, the BMPER:RGM-A heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 39-50 of SEQ ID NO: 553 and ends at any one of amino acids 364-369 of SEQ ID NO: 553. In some embodiments, the BMPER:RGM-A heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 370-386 of SEQ ID NO: 553 and ends at any one of amino acids 682-685 of SEQ ID NO: 553. In some embodiments, the BMPER:RGM-A heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 39-50 of SEQ ID NO: 553 and ends at any one of amino acids 682-685 of SEQ ID NO: 553. In some embodiments, the BMPER:RGM-A heteromultimer comprises a BMPER protein, wherein the BMPER protein is a dimer comprising a first polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 39-50 of SEQ ID NO: 553 and ends at any one of amino acids 364-369 of SEQ ID NO: 553, and second polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 370-386 of SEQ ID NO: 553, and ends at any one of amino acids 682-685 of SEQ ID NO: 553. In some embodiments, the BMPER:RGM-A heteromultimer comprises a single chain ligand trap that comprises a first BMPER polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 39-50 of SEQ ID NO: 553 and ends at any one of amino acids 364-369 of SEQ ID NO: 553, and second BMPER polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 370-386 of SEQ ID NO: 553 and ends at any one of amino acids 682-685 of SEQ ID NO: 553. In some embodiments, the BMPER:RGM-A heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 553 or 554. In some embodiments, the BMPER:RGM-A heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-177 of SEQ ID NO: 561 and ends at any one of amino acids 430-458 of SEQ ID NO: 561. In some embodiments, the BMPER:RGM-A heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-153 of SEQ ID NO: 565 and ends at any one of amino acids 406-434 of SEQ ID NO: 565. In some embodiments, the BMPER:RGM-A heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-169 of SEQ ID NO: 569 and ends at any one of amino acids 422-450 of SEQ ID NO: 569. In certain preferred embodiments, BMPER:RGM-A heteromultimers are soluble. In some embodiments, a BMPER:RGM-A heteromultimer of the disclosure binds to one or more TGF-beta superfamily ligands (e.g., binds to one or more TGF-beta superfamily ligands with a K_{D} of at least 1×10⁻⁷). In some embodiments, a BMPER:RGM-A heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a BMPER:RGM-A heteromultimer of the disclosure has a different TGF-beta ligand binding and/or inhibition profile (specificity) compared to a corresponding homomultimer (e.g., BMPER and RGM-A homomultimers). In some embodiments, a BMPER:RGM-A heteromultimer of the disclosure is a heterodimer.

In certain aspects, the disclosure relates to heteromultimers that comprise at least one BMPER polypeptide, which includes fragments, functional variants, and modified forms thereof, and at least one RGM-B polypeptide, which includes fragments, functional variants, and modified forms thereof. In some embodiments, the BMPER:RGM-B heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 553 or 554. In some embodiments, the BMPER:RGM-B heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 39-50 of SEQ ID NO: 553 and ends at any one of amino acids 364-369 of SEQ ID NO: 553. In some embodiments, the BMPER:RGM-B heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 370-386 of SEQ ID NO: 553 and ends at any one of amino acids 682-685 of SEQ ID NO: 553. In some embodiments, the BMPER:RGM-B heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 39-50 of SEQ ID NO: 553 and ends at any one of amino acids 682-685 of SEQ ID NO: 553. In some embodiments, the BMPER:RGM-B heteromultimer comprises a BMPER protein, wherein the BMPER protein is a dimer comprising a first polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 39-50 of SEQ ID NO: 553 and ends at any one of amino acids 364-369 of SEQ ID NO: 553, and second polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 370-386 of SEQ ID NO: 553, and ends at any one of amino acids 682-685 of SEQ ID NO: 553. In some embodiments, the BMPER:RGM-B heteromultimer comprises a single chain ligand trap that comprises a first BMPER polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 39-50 of SEQ ID NO: 553 and ends at any one of amino acids 364-369 of SEQ ID NO: 553, and second BMPER polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 370-386 of SEQ ID NO: 553 and ends at any one of amino acids 682-685 of SEQ ID NO: 553. In some embodiments, the BMPER:RGM-B heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 557 or 558. In some embodiments, the BMPER:RGM-B heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-87 of SEQ ID NO: 557 and ends at any one of amino acids 452-478 of SEQ ID NO: 557. In some embodiments, the BMPER:RGM-B heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 210-222 of SEQ ID NO: 557 and ends at any one of amino acids 413-452 of SEQ ID NO: 557. In some embodiments, the BMPER:RGM-B heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 87-95 of SEQ ID NO: 557 and ends at any one of amino acids 204-209 of SEQ ID NO: 557. In some embodiments, the BMPER:RGM-B heteromultimer comprises a RGM-B protein, wherein the RGM-B protein is a dimer comprising a first polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 87-95 of SEQ ID NO: 557 and ends at any one of amino acids 204-209 of SEQ ID NO: 557 and second polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 210-222 of SEQ ID NO: 557 and ends at any one of amino acids 413-452 of SEQ ID NO: 557. In some embodiments, the BMPER:RGM-B heteromultimer comprises a single chain ligand trap that comprises a first RGM-B polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 87-95 of SEQ ID NO: 557 and ends at any one of amino acids 204-209 of SEQ ID NO: 557 and second RGM-B polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 210-222 of SEQ ID NO: 557 and ends at any one of amino acids 413-452 of SEQ ID NO: 557. In certain preferred embodiments, BMPER:RGM-B heteromultimers are soluble. In some embodiments, a BMPER:RGM-B heteromultimer of the disclosure binds to one or more TGF-beta superfamily ligands (e.g., binds to one or more TGF-beta superfamily ligands with a K_{D} of at least 1×10⁻⁷). In some embodiments, a BMPER:RGM-B heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a BMPER:RGM-B heteromultimer of the disclosure has a different TGF-beta ligand binding and/or inhibition profile (specificity) compared to a corresponding homomultimer (e.g., BMPER and RGM-B homomultimers). In some embodiments, a BMPER:RGM-B heteromultimer of the disclosure is a heterodimer.

In certain aspects, the disclosure relates to heteromultimers that comprise at least one BMPER polypeptide, which includes fragments, functional variants, and modified forms thereof, and at least one hemojuvelin polypeptide, which includes fragments, functional variants, and modified forms thereof. In some embodiments, the BMPER:hemojuvelin heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 553 or 554. In some embodiments, the BMPER:hemojuvelin heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 39-50 of SEQ ID NO: 553 and ends at any one of amino acids 364-369 of SEQ ID NO: 553. In some embodiments, the BMPER:hemojuvelin heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 370-386 of SEQ ID NO: 553 and ends at any one of amino acids 682-685 of SEQ ID NO: 553. In some embodiments, the BMPER:hemojuvelin heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 39-50 of SEQ ID NO: 553 and ends at any one of amino acids 682-685 of SEQ ID NO: 553. In some embodiments, the BMPER:hemojuvelin heteromultimer comprises a BMPER protein, wherein the BMPER protein is a dimer comprising a first polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 39-50 of SEQ ID NO: 553 and ends at any one of amino acids 364-369 of SEQ ID NO: 553, and second polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 370-386 of SEQ ID NO: 553, and ends at any one of amino acids 682-685 of SEQ ID NO: 553. In some embodiments, the BMPER:hemojuvelin heteromultimer comprises a single chain ligand trap that comprises a first BMPER polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 39-50 of SEQ ID NO: 553 and ends at any one of amino acids 364-369 of SEQ ID NO: 553, and second BMPER polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 370-386 of SEQ ID NO: 553 and ends at any one of amino acids 682-685 of SEQ ID NO: 553. In some embodiments, the BMPER:hemojuvelin heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 573, 574, 577, 578, 581, or 582. In some embodiments, the BMPER:hemojuvelin heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-36 of SEQ ID NO: 573 and ends at any one of amino acids 400-426 of SEQ ID NO: 573. In some embodiments, the BMPER:hemojuvelin heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 36-42 of SEQ ID NO: 573 and ends at any one of amino acids 167-172 of SEQ ID NO: 573. In some embodiments, the BMPER:hemojuvelin heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 173-185 of SEQ ID NO: 573 and ends at any one of amino acids 361-400 of SEQ ID NO: 573. In some embodiments, the BMPER:hemojuvelin heteromultimer comprises a hemojuvelin protein that is a dimer comprising a first polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 36-42 of SEQ ID NO: 573, and ends at any one of amino acids 167-172 of SEQ ID NO: 573 and second polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 173-185 of SEQ ID NO: 573 and ends at any one of amino acids 361-400 of SEQ ID NO: 573. In some embodiments, the BMPER:hemojuvelin heteromultimer comprises a single chain ligand trap that comprises a first hemojuvelin polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 36-42 of SEQ ID NO: 573 and ends at any one of amino acids 167-172 of SEQ ID NO: 573 and second hemojuvelin polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 173-185 of SEQ ID NO: 573 and ends at any one of amino acids 361-400 of SEQ ID NO: 573. In some embodiments, the BMPER:hemojuvelin heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-6 of SEQ ID NO: 577 and ends at any one of amino acids 287-313 of SEQ ID NO: 577. In some embodiments, the BMPER:hemojuvelin heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-6 of SEQ ID NO: 577 and ends at any one of amino acids 54-59 of SEQ ID NO: 577. In some embodiments, the BMPER:hemojuvelin heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 60-72 of SEQ ID NO: 577 and ends at any one of amino acids 248-287 of SEQ ID NO: 577. In some embodiments, the BMPER:hemojuvelin heteromultimer comprises a hemojuvelin protein, wherein the hemojuvelin protein is a dimer comprising a first polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 1-6 of SEQ ID NO: 577 and ends at any one of amino acids 54-59 of SEQ ID NO: 577, and second polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 60-72 of SEQ ID NO: 577 and ends at any one of amino acids 248-287 of SEQ ID NO: 577. In some embodiments, the BMPER:hemojuvelin heteromultimer comprises a single chain ligand trap that comprises a first hemojuvelin polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 1-6 of SEQ ID NO: 577 and ends at any one of amino acids 54-59 of SEQ ID NO: 577, and second hemojuvelin polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 60-72 of SEQ ID NO: 577, and ends at any one of amino acids 248-287 of SEQ ID NO: 577. In some embodiments, the BMPER:hemojuvelin heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-4 of SEQ ID NO: 581 and ends at any one of amino acids 135-200 of SEQ ID NO: 581. In certain preferred embodiments, BMPER:hemojuvelin heteromultimers are soluble. In some embodiments, a BMPER:hemojuvelin heteromultimer of the disclosure binds to one or more TGF-beta superfamily ligands (e.g., binds to one or more TGF-beta superfamily ligands with a K_{D} of at least 1×10⁻⁷). In some embodiments, a BMPER:hemojuvelin heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a BMPER:hemojuvelin heteromultimer of the disclosure has a different TGF-beta ligand binding and/or inhibition profile (specificity) compared to a corresponding homomultimer (e.g., BMPER and hemojuvelin homomultimers). In some embodiments, a BMPER:hemojuvelin heteromultimer of the disclosure is a heterodimer.

In certain aspects, the disclosure relates to heteromultimers that comprise at least one RGM-A polypeptide, which includes fragments, functional variants, and modified forms thereof, and at least one RGM-B polypeptide, which includes fragments, functional variants, and modified forms thereof. In some embodiments, the RGM-A:RGM-B heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 553 or 554. In some embodiments, the RGM-A:RGM-B heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-177 of SEQ ID NO: 561 and ends at any one of amino acids 430-458 of SEQ ID NO: 561. In some embodiments, the RGM-A:RGM-B heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-153 of SEQ ID NO: 565 and ends at any one of amino acids 406-434 of SEQ ID NO: 565. In some embodiments, the RGM-A:RGM-B heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-169 of SEQ ID NO: 569 and ends at any one of amino acids 422-450 of SEQ ID NO: 569. In some embodiments, the RGM-A:RGM-B heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 557 or 558. In some embodiments, the RGM-A:RGM-B heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-87 of SEQ ID NO: 557 and ends at any one of amino acids 452-478 of SEQ ID NO: 557. In some embodiments, the RGM-A:RGM-B heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 210-222 of SEQ ID NO: 557 and ends at any one of amino acids 413-452 of SEQ ID NO: 557. In some embodiments, the RGM-A:RGM-B heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 87-95 of SEQ ID NO: 557 and ends at any one of amino acids 204-209 of SEQ ID NO: 557. In some embodiments, the RGM-A:RGM-B heteromultimer comprises a RGM-B protein, wherein the RGM-B protein is a dimer comprising a first polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 87-95 of SEQ ID NO: 557 and ends at any one of amino acids 204-209 of SEQ ID NO: 557 and second polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 210-222 of SEQ ID NO: 557 and ends at any one of amino acids 413-452 of SEQ ID NO: 557. In some embodiments, the RGM-A:RGM-B heteromultimer comprises a single chain ligand trap that comprises a first RGM-B polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 87-95 of SEQ ID NO: 557 and ends at any one of amino acids 204-209 of SEQ ID NO: 557 and second RGM-B polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 210-222 of SEQ ID NO: 557 and ends at any one of amino acids 413-452 of SEQ ID NO: 557. In certain preferred embodiments, RGM-A:RGM-B heteromultimers are soluble. In some embodiments, a RGM-A:RGM-B heteromultimer of the disclosure binds to one or more TGF-beta superfamily ligands (e.g., binds to one or more TGF-beta superfamily ligands with a K_{D} of at least 1×10⁻⁷). In some embodiments, a RGM-A:RGM-B heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a RGM-A:RGM-B heteromultimer of the disclosure has a different TGF-beta ligand binding and/or inhibition profile (specificity) compared to a corresponding homomultimer (e.g., RGM-A and RGM-B homomultimers). In some embodiments, a RGM-A:RGM-B heteromultimer of the disclosure is a heterodimer.

In certain aspects, the disclosure relates to heteromultimers that comprise at least one RGM-A polypeptide, which includes fragments, functional variants, and modified forms thereof, and at least one hemojuvelin polypeptide, which includes fragments, functional variants, and modified forms thereof. In some embodiments, the RGM-A:hemojuvelin heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 553 or 554. In some embodiments, the RGM-A:hemojuvelin heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-177 of SEQ ID NO: 561 and ends at any one of amino acids 430-458 of SEQ ID NO: 561. In some embodiments, the RGM-A:hemojuvelin heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-153 of SEQ ID NO: 565 and ends at any one of amino acids 406-434 of SEQ ID NO: 565. In some embodiments, the RGM-A:hemojuvelin heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-169 of SEQ ID NO: 569 and ends at any one of amino acids 422-450 of SEQ ID NO: 569. In some embodiments, the RGM-A:hemojuvelin heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 573, 574, 577, 578, 581, or 582. In some embodiments, the RGM-A:hemojuvelin heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-36 of SEQ ID NO: 573 and ends at any one of amino acids 400-426 of SEQ ID NO: 573. In some embodiments, the RGM-A:hemojuvelin heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 36-42 of SEQ ID NO: 573 and ends at any one of amino acids 167-172 of SEQ ID NO: 573. In some embodiments, the RGM-A:hemojuvelin heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 173-185 of SEQ ID NO: 573 and ends at any one of amino acids 361-400 of SEQ ID NO: 573. In some embodiments, the RGM-A:hemojuvelin heteromultimer comprises a hemojuvelin protein that is a dimer comprising a first polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 36-42 of SEQ ID NO: 573, and ends at any one of amino acids 167-172 of SEQ ID NO: 573 and second polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 173-185 of SEQ ID NO: 573 and ends at any one of amino acids 361-400 of SEQ ID NO: 573. In some embodiments, the RGM-A:hemojuvelin heteromultimer comprises a single chain ligand trap that comprises a first hemojuvelin polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 36-42 of SEQ ID NO: 573 and ends at any one of amino acids 167-172 of SEQ ID NO: 573 and second hemojuvelin polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 173-185 of SEQ ID NO: 573 and ends at any one of amino acids 361-400 of SEQ ID NO: 573. In some embodiments, the RGM-A:hemojuvelin heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-6 of SEQ ID NO: 577 and ends at any one of amino acids 287-313 of SEQ ID NO: 577. In some embodiments, the RGM-A:hemojuvelin heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-6 of SEQ ID NO: 577 and ends at any one of amino acids 54-59 of SEQ ID NO: 577. In some embodiments, the RGM-A:hemojuvelin heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 60-72 of SEQ ID NO: 577 and ends at any one of amino acids 248-287 of SEQ ID NO: 577. In some embodiments, the RGM-A:hemojuvelin heteromultimer comprises a hemojuvelin protein, wherein the hemojuvelin protein is a dimer comprising a first polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 1-6 of SEQ ID NO: 577 and ends at any one of amino acids 54-59 of SEQ ID NO: 577, and second polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 60-72 of SEQ ID NO: 577 and ends at any one of amino acids 248-287 of SEQ ID NO: 577. In some embodiments, the RGM-A:hemojuvelin heteromultimer comprises a single chain ligand trap that comprises a first hemojuvelin polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 1-6 of SEQ ID NO: 577 and ends at any one of amino acids 54-59 of SEQ ID NO: 577, and second hemojuvelin polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 60-72 of SEQ ID NO: 577, and ends at any one of amino acids 248-287 of SEQ ID NO: 577. In some embodiments, the RGM-A:hemojuvelin heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-4 of SEQ ID NO: 581 and ends at any one of amino acids 135-200 of SEQ ID NO: 581. In certain preferred embodiments, RGM-A:hemojuvelin heteromultimers are soluble. In some embodiments, a RGM-A:hemojuvelin heteromultimer of the disclosure binds to one or more TGF-beta superfamily ligands (e.g., binds to one or more TGF-beta superfamily ligands with a K_{D} of at least 1×10⁻⁷). In some embodiments, a RGM-A:hemojuvelin heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a RGM-A:hemojuvelin heteromultimer of the disclosure has a different TGF-beta ligand binding and/or inhibition profile (specificity) compared to a corresponding homomultimer (e.g., RGM-A and hemojuvelin homomultimers). In some embodiments, a RGM-A:hemojuvelin heteromultimer of the disclosure is a heterodimer.

In certain aspects, the disclosure relates to heteromultimers that comprise at least one RGM-B polypeptide, which includes fragments, functional variants, and modified forms thereof, and at least one hemojuvelin polypeptide, which includes fragments, functional variants, and modified forms thereof. In some embodiments, the RGM-B:hemojuvelin heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 557 or 558. In some embodiments, the RGM-B:hemojuvelin heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-87 of SEQ ID NO: 557 and ends at any one of amino acids 452-478 of SEQ ID NO: 557. In some embodiments, the RGM-B:hemojuvelin heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 210-222 of SEQ ID NO: 557 and ends at any one of amino acids 413-452 of SEQ ID NO: 557. In some embodiments, the RGM-B:hemojuvelin heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 87-95 of SEQ ID NO: 557 and ends at any one of amino acids 204-209 of SEQ ID NO: 557. In some embodiments, the RGM-B:hemojuvelin heteromultimer comprises a RGM-B protein, wherein the RGM-B protein is a dimer comprising a first polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 87-95 of SEQ ID NO: 557 and ends at any one of amino acids 204-209 of SEQ ID NO: 557 and second polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 210-222 of SEQ ID NO: 557 and ends at any one of amino acids 413-452 of SEQ ID NO: 557. In some embodiments, the RGM-B:hemojuvelin heteromultimer comprises a single chain ligand trap that comprises a first RGM-B polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 87-95 of SEQ ID NO: 557 and ends at any one of amino acids 204-209 of SEQ ID NO: 557 and second RGM-B polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 210-222 of SEQ ID NO: 557 and ends at any one of amino acids 413-452 of SEQ ID NO: 557. In some embodiments, the RGM-B:hemojuvelin heteromultimer comprises a polypeptide that is at least 70%, 75%, 80%, 85%, 90%, 91%, 92%, 93%, 94%, 95%, 95%, 96%, 97%, 98%, 99%, or 100% identical to the amino acid sequence of any one of SEQ ID NOs: 573, 574, 577, 578, 581, or 582. In some embodiments, the RGM-B:hemojuvelin heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-36 of SEQ ID NO: 573 and ends at any one of amino acids 400-426 of SEQ ID NO: 573. In some embodiments, the RGM-B:hemojuvelin heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 36-42 of SEQ ID NO: 573 and ends at any one of amino acids 167-172 of SEQ ID NO: 573. In some embodiments, the RGM-B:hemojuvelin heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 173-185 of SEQ ID NO: 573 and ends at any one of amino acids 361-400 of SEQ ID NO: 573. In some embodiments, the RGM-B:hemojuvelin heteromultimer comprises a hemojuvelin protein that is a dimer comprising a first polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 36-42 of SEQ ID NO: 573, and ends at any one of amino acids 167-172 of SEQ ID NO: 573 and second polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 173-185 of SEQ ID NO: 573 and ends at any one of amino acids 361-400 of SEQ ID NO: 573. In some embodiments, the RGM-B:hemojuvelin heteromultimer comprises a single chain ligand trap that comprises a first hemojuvelin polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 36-42 of SEQ ID NO: 573 and ends at any one of amino acids 167-172 of SEQ ID NO: 573 and second hemojuvelin polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 173-185 of SEQ ID NO: 573 and ends at any one of amino acids 361-400 of SEQ ID NO: 573. In some embodiments, the RGM-B:hemojuvelin heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-6 of SEQ ID NO: 577 and ends at any one of amino acids 287-313 of SEQ ID NO: 577. In some embodiments, the RGM-B:hemojuvelin heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-6 of SEQ ID NO: 577 and ends at any one of amino acids 54-59 of SEQ ID NO: 577. In some embodiments, the RGM-B:hemojuvelin heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 60-72 of SEQ ID NO: 577 and ends at any one of amino acids 248-287 of SEQ ID NO: 577. In some embodiments, the RGM-B:hemojuvelin heteromultimer comprises a hemojuvelin protein, wherein the hemojuvelin protein is a dimer comprising a first polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 1-6 of SEQ ID NO: 577 and ends at any one of amino acids 54-59 of SEQ ID NO: 577, and second polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 60-72 of SEQ ID NO: 577 and ends at any one of amino acids 248-287 of SEQ ID NO: 577. In some embodiments, the RGM-B:hemojuvelin heteromultimer comprises a single chain ligand trap that comprises a first hemojuvelin polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 1-6 of SEQ ID NO: 577 and ends at any one of amino acids 54-59 of SEQ ID NO: 577, and second hemojuvelin polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 60-72 of SEQ ID NO: 577, and ends at any one of amino acids 248-287 of SEQ ID NO: 577. In some embodiments, the RGM-B:hemojuvelin heteromultimer comprises a polypeptide that is at least 70%, 75%, 80%, 85%, 90%, 91%, 92%, 93%, 94%, 95%, 95%, 96%, 97%, 98%, 99%, or 100% identical to a polypeptide that begins at any one of amino acids of 1-4 of SEQ ID NO: 581 and ends at any one of amino acids 135-200 of SEQ ID NO: 581. In certain preferred embodiments, RGM-B:hemojuvelin heteromultimers are soluble. In some embodiments, a RGM-B:hemojuvelin heteromultimer of the disclosure binds to one or more TGF-beta superfamily ligands (e.g., binds to one or more TGF-beta superfamily ligands with a K_{D} of at least 1×10⁻⁷). In some embodiments, a RGM-B:hemojuvelin heteromultimer of the disclosure inhibits one or more TGF-beta superfamily ligands (e.g., inhibits Smad signaling). Heteromultimer-ligand binding and inhibition may be determined using a variety of assays including, for example, those described herein (e.g., in vitro binding and/or cell-based signaling assays). In some embodiments, a RGM-B:hemojuvelin heteromultimer of the disclosure has a different TGF-beta ligand binding and/or inhibition profile (specificity) compared to a corresponding homomultimer (e.g., RGM-B and hemojuvelin homomultimers). In some embodiments, a RGM-B:hemojuvelin heteromultimer of the disclosure is a heterodimer.

In certain aspects, a TGF-beta superfamily type I receptor polypeptide, TGF-beta superfamily type II receptor polypeptide, and/or TGF-beta superfamily co-receptor polypeptide of the disclosure is a fusion protein further comprising polypeptide domain that is heterologous (a heterologous polypeptide domain) to the TGF-beta superfamily type I receptor polypeptide domain, TGF-beta superfamily type II receptor polypeptide domain, and/or TGF-beta superfamily co-receptor polypeptide domain. In some embodiments, a TGF-beta superfamily type I receptor polypeptide is a fusion protein further comprising a heterologous polypeptide domain that is a first or second member of an interaction pair. In some embodiments, a TGF-beta superfamily type II receptor polypeptide is a fusion protein further comprising a heterologous polypeptide domain that is a first or second member of an interaction pair. In some embodiments, a TGF-beta superfamily co-receptor polypeptide is a fusion protein further comprising a heterologous polypeptide domain that is a first or second member of an interaction pair. In certain embodiments, heteromultimers described herein comprise a first polypeptide covalently or non-covalently associated with a second polypeptide wherein the first polypeptide comprises the amino acid sequence of a TGF-beta superfamily type I receptor polypeptide and the amino acid sequence of a first member of an interaction pair (or a second member of an interaction pair) and the second polypeptide comprises the amino acid sequence of a TGF-beta superfamily co-receptor polypeptide and the amino acid sequence of a second member of the interaction pair (or a first member of the interaction pair). In certain embodiments, heteromultimers described herein comprise a first polypeptide covalently or non-covalently associated with a second polypeptide wherein the first polypeptide comprises the amino acid sequence of a TGF-beta superfamily type II receptor polypeptide and the amino acid sequence of a first member of an interaction pair (or a second member of an interaction pair) and the second polypeptide comprises the amino acid sequence of a TGF-beta superfamily co-receptor polypeptide and the amino acid sequence of a second member of the interaction pair (or a first member of the interaction pair). In certain embodiments, heteromultimers described herein comprise a first polypeptide covalently or non-covalently associated with a second polypeptide wherein the first polypeptide comprises the amino acid sequence of a first TGF-beta superfamily co-receptor polypeptide and the amino acid sequence of a first member of an interaction pair (or a second member of an interaction pair) and the second polypeptide comprises the amino acid sequence of a second TGF-beta superfamily co-receptor polypeptide and the amino acid sequence of a second member of the interaction pair (or a first member of the interaction pair). Optionally, the TGF-beta superfamily type I receptor polypeptide is connected directly to the first member of the interaction pair, or an intervening sequence, such as a linker, may be positioned between the amino acid sequence of the TGF-beta superfamily type I receptor polypeptide and the amino acid sequence of the first member of the interaction pair. Similarly, the TGF-beta superfamily type II receptor polypeptide may be connected directly to the second member of the interaction pair, or an intervening sequence, such as a linker, may be positioned between the amino acid sequence of the TGF-beta superfamily type II receptor polypeptide and the amino acid sequence of the second member of the interaction pair. Similarly, the TGF-beta superfamily co-receptor polypeptide may be connected directly to the second member of the interaction pair, or an intervening sequence, such as a linker, may be positioned between the amino acid sequence of the TGF-beta superfamily co-receptor polypeptide and the amino acid sequence of the second member of the interaction pair. Examples of linkers include, but are not limited to, the sequences TGGG (SEQ ID NO: 62), TGGGG (SEQ ID NO: 60), SGGGG (SEQ ID NO: 61), GGGG (SEQ ID NO: 59) SGGG (SEQ ID NO: 63), and GGG (SEQ ID NO: 58).

Interaction pairs described herein are designed to promote dimerization or form higher order multimers. In some embodiments, the interaction pair may be any two polypeptide sequences that interact to form a complex, particularly a heterodimeric complex although operative embodiments may also employ an interaction pair that forms a homodimeric sequence. The first and second members of the interaction pair may be an asymmetric pair, meaning that the members of the pair preferentially associate with each other rather than self-associate. Accordingly, first and second members of an asymmetric interaction pair may associate to form a heterodimeric complex. Alternatively, the interaction pair may be unguided, meaning that the members of the pair may associate with each other or self-associate without substantial preference and thus may have the same or different amino acid sequences. Accordingly, first and second members of an unguided interaction pair may associate to form a homodimer complex or a heterodimeric complex. Optionally, the first member of the interaction action pair (e.g., an asymmetric pair or an unguided interaction pair) associates covalently with the second member of the interaction pair. Optionally, the first member of the interaction action pair (e.g., an asymmetric pair or an unguided interaction pair) associates non-covalently with the second member of the interaction pair. Optionally, the first member of the interaction pair (e.g., an asymmetrical or an unguided interaction pair) associates through both covalent and non-covalent mechanisms with the second member of the interaction pair.

In certain aspects, a TGF-beta superfamily type I receptor polypeptide, TGF-beta superfamily type II receptor polypeptide, and/or TGF-beta superfamily co-receptor polypeptide is a fusion protein that comprises constant region from an IgG heavy chain. In some embodiments, the constant region from an IgG heavy chain is an immunoglobulin Fc domain. Traditional Fc fusion proteins and antibodies are examples of unguided interaction pairs, whereas a variety of engineered Fc domains have been designed as asymmetric interaction pairs [Spiess et al (2015) Molecular Immunology 67(2A): 95-106]. Therefore, a first member and/or a second member of an interaction pair described herein may comprise a constant domain of an immunoglobulin, including, for example, the Fc portion of an immunoglobulin. For example, a first member of an interaction pair may comprise an amino acid sequence that is derived from an Fc domain of an IgG (IgG1, IgG2, IgG3, or IgG4), IgA (IgA1 or IgA2), IgE, or IgM immunoglobulin. For example, the first member of an interaction pair may comprise, consist essentially of, or consist of an amino acid sequence that is at least 80%, 85%, 90%, 91%, 92%, 93%, 94%, 95%, 96%, 97%, 98%, 99% or 100% identical to any one of SEQ ID NOs: 200-217. Optionally, a second member of an interaction pair may comprise an amino acid sequence that is derived from an Fc domain of an IgG (IgG1, IgG2, IgG3, or IgG4), IgA (IgA1 or IgA2), IgE, or IgM. Such immunoglobulin domains may comprise one or more amino acid modifications (e.g., deletions, additions, and/or substitutions) that promote heterodimer formation. For example, the second member of an interaction pair may comprise, consist essentially of, or consist of an amino acid sequence that is at least 80%, 85%, 90%, 91%, 92%, 93%, 94%, 95%, 96%, 97%, 98%, 99% or 100% identical to any one of SEQ ID NOs: 200-217. In some embodiments, a first member and a second member of an interaction pair comprise Fc domains derived from the same immunoglobulin class and subtype. In other embodiments, a first member and a second member of an interaction pair comprise Fc domains derived from different immunoglobulin classes or subtypes. Similarly, a first member and/or a second member of an interaction pair (e.g., an asymmetric pair or an unguided interaction pair) comprise a modified constant domain of an immunoglobulin, including, for example, a modified Fc portion of an immunoglobulin. For example, protein complexes of the disclosure may comprise a first modified Fc portion of an immunoglobulin comprising an amino acid sequence that is at least 80%, 85%, 90%, 91%, 92%, 93%, 94%, 95%, 96%, 97%, 98%, or 99% identical to an amino acid sequence selected from the group: SEQ ID NOs: 200-217 and a second modified Fc portion of an immunoglobulin, which may be the same or different from the amino acid sequence of the first modified Fc portion of the immunoglobulin, comprising an amino acid sequence that is at least 80%, 85%, 90%, 91%, 92%, 93%, 94%, 95%, 96%, 97%, 98%, or 99% identical to an amino acid sequence selected from the group: SEQ ID NOs: 200-217. Such immunoglobulin domains may comprise one or more amino acid modifications (e.g., deletions, additions, and/or substitutions) that promote heteromultimer formation. Such immunoglobulin domains may comprise one or more amino acid modifications (e.g., deletions, additions, and/or substitutions) that inhibit homomultimer formation. In some embodiments, a heteromultimer of the disclosure comprises a TGF-beta superfamily type I receptor polypeptide that is a fusion protein further comprising a heterologous polypeptide domain, and wherein the heterologous polypeptide domain comprises an amino acid sequence that is 70%, 75%, 80%, 85%, 90%, 91%, 92%, 93%, 94%, 95%, 96%, 97%, 98%, 99%, or 100% identical to the amino acid sequence of any one of SEQ ID Nos: 200, 202, 204, 206, 213, 215, or 217, and TGF-beta superfamily co-receptor polypeptide that is a fusion protein further comprising a heterologous polypeptide domain, and wherein the heterologous polypeptide domain comprises an amino acid sequence that is 70%, 75%, 80%, 85%, 90%, 91%, 92%, 93%, 94%, 95%, 96%, 97%, 98%, 99%, or 100% identical to the amino acid sequence of any one of SEQ ID Nos: 201, 203, 205, 207, 214, or 216. In some embodiments, heteromultimer of the disclosure comprise TGF-beta superfamily co-receptor polypeptide that is a fusion protein further comprising a heterologous polypeptide domain, and wherein the heterologous polypeptide domain comprises an amino acid sequence that is 70%, 75%, 80%, 85%, 90%, 91%, 92%, 93%, 94%, 95%, 96%, 97%, 98%, 99%, or 100% identical to the amino acid sequence of any one of SEQ ID Nos: 200, 202, 204, 206, 213, 215 or 217; and TGF-beta superfamily type I receptor polypeptide that is a fusion protein further comprising a heterologous polypeptide domain, and wherein the heterologous polypeptide domain comprises an amino acid sequence that is 70%, 75%, 80%, 85%, 90%, 91%, 92%, 93%, 94%, 95%, 96%, 97%, 98%, 99%, or 100% identical to the amino acid sequence of any one of SEQ ID Nos: 201, 203, 205, 207, 214, or 216. In some embodiments, a heteromultimer of the disclosure comprises a TGF-beta superfamily type II receptor polypeptide that is a fusion protein further comprising a heterologous polypeptide domain, and wherein the heterologous polypeptide domain comprises an amino acid sequence that is 70%, 75%, 80%, 85%, 90%, 91%, 92%, 93%, 94%, 95%, 96%, 97%, 98%, 99%, or 100% identical to the amino acid sequence of any one of SEQ ID Nos: 200, 202, 204, 206, 213, 215, or 217; and a TGF-beta superfamily co-receptor polypeptide that is a fusion protein further comprising a heterologous polypeptide domain, and wherein the heterologous polypeptide domain comprises an amino acid sequence that is 70%, 75%, 80%, 85%, 90%, 91%, 92%, 93%, 94%, 95%, 96%, 97%, 98%, 99%, or 100% identical to the amino acid sequence of any one of SEQ ID Nos: 201, 203, 205, 207, 214 and 216. In some embodiments, a heteromultimer of the disclosure comprises a TGF-beta superfamily co-receptor polypeptide that is a fusion protein further comprising a heterologous polypeptide domain, and wherein the heterologous polypeptide domain comprises an amino acid sequence that is 70%, 75%, 80%, 85%, 90%, 91%, 92%, 93%, 94%, 95%, 96%, 97%, 98%, 99%, or 100% identical to the amino acid sequence of any one of SEQ ID Nos: 200, 202, 204, 206, 213, 215, or 217; and a TGF-beta superfamily type II receptor polypeptide that is a fusion protein further comprising a heterologous polypeptide domain, and wherein the heterologous polypeptide domain comprises an amino acid sequence that is 70%, 75%, 80%, 85%, 90%, 91%, 92%, 93%, 94%, 95%, 96%, 97%, 98%, 99%, or 100% identical to the amino acid sequence of any one of SEQ ID Nos: 201, 203, 205, 207, 214, or 216. In some embodiments, a heteromultimer of the disclosure comprises a first TGF-beta superfamily co-receptor polypeptide that is a fusion protein further comprising a heterologous polypeptide domain, and wherein the heterologous polypeptide domain comprises an amino acid sequence that is 70%, 75%, 80%, 85%, 90%, 91%, 92%, 93%, 94%, 95%, 96%, 97%, 98%, 99%, or 100% identical to the amino acid sequence of any one of SEQ ID Nos: 200, 202, 204, 206, 213, 215, or 217; and a second TGF-beta superfamily co-receptor polypeptide that is a fusion protein further comprising a heterologous polypeptide domain, and wherein the heterologous polypeptide domain comprises an amino acid sequence that is 70%, 75%, 80%, 85%, 90%, 91%, 92%, 93%, 94%, 95%, 96%, 97%, 98%, 99%, or 100% identical to the amino acid sequence of any one of SEQ ID Nos: 201, 203, 205, 207, 214, or 216. In some embodiments, a heteromultimer of the disclosure comprises a second TGF-beta superfamily co-receptor polypeptide that is a fusion protein further comprising a heterologous polypeptide domain, and wherein the heterologous polypeptide domain comprises an amino acid sequence that is 70%, 75%, 80%, 85%, 90%, 91%, 92%, 93%, 94%, 95%, 96%, 97%, 98%, 99%, or 100% identical to the amino acid sequence of any one of SEQ ID Nos: 200, 202, 204, 206, 213, 215, or 217; and a first TGF-beta superfamily co-receptor polypeptide that is a fusion protein further comprising a heterologous polypeptide domain, and wherein the heterologous polypeptide domain comprises an amino acid sequence that is 70%, 75%, 80%, 85%, 90%, 91%, 92%, 93%, 94%, 95%, 96%, 97%, 98%, 99%, or 100% identical to the amino acid sequence of any one of SEQ ID Nos: 201, 203, 205, 207, 214 and 216.

In certain aspects, a TGF-beta superfamily type I receptor polypeptide, TGF-beta superfamily type II receptor polypeptide, and/or TGF-beta superfamily co-receptor polypeptide disclosed herein comprise one or more modified amino acid residues selected from: a glycosylated amino acid, a PEGylated amino acid, a farnesylated amino acid, an acetylated amino acid, a biotinylated amino acid, an amino acid conjugated to a lipid moiety, and an amino acid conjugated to an organic derivatizing agent. In some embodiments, a TGF-beta superfamily type I receptor polypeptide, TGF-beta superfamily type II receptor polypeptide, and/or TGF-beta superfamily co-receptor polypeptide is glycosylated and has a glycosylation pattern obtainable from the expression of the polypeptides in a mammalian cell, including, for example, a CHO cell.

In certain aspects, the disclosure provides nucleic acids (e.g., isolated nucleic acids and/or recombinant nucleic acids) encoding any of the TGF-beta superfamily type I receptor polypeptides, TGF-beta superfamily type II receptor polypeptides, and/or TGF-beta superfamily co-receptor polypeptides described herein. In some embodiments, one or more of the nucleic acids disclosed herein may be operably linked to a promoter for expression. In some embodiments, the disclosure provides vectors that comprise one or more of the nucleic acids disclosed herein. In some embodiments, the disclosure provides cells that comprise one or more of the nucleic acids or vectors disclosed herein. Preferably the cell is a mammalian cell such as a COS cell or a CHO cell.

In certain aspects, the disclosure provides methods for making one or more of the TGF-beta superfamily type I receptor polypeptides, TGF-beta superfamily type II receptor polypeptides, and/or TGF-beta superfamily co-receptor polypeptides described herein as well as heteromultimers comprising such polypeptides. Such methods may include expressing any of the nucleic acids disclosed herein in a suitable cell (e.g., CHO cell or a COS cell). In some embodiments, the disclosure relates to a method of making a heteromultimer comprising a TGF-beta type I receptor polypeptide and a TGF-beta co-receptor polypeptide by culturing a cell under conditions suitable for expression of a TGF-beta type I receptor polypeptide and a TGF-beta co-receptor polypeptide, wherein the cell comprises a first nucleic acid comprising a coding sequence for a TGF-beta type I receptor polypeptide, such as those described herein, and a second nucleic acid comprising a coding sequence for a TGF-beta co-receptor, such as those described herein. In some embodiments, the disclosure relates to a method of making a heteromultimer comprising a TGF-beta type II receptor polypeptide and a TGF-beta co-receptor polypeptide by culturing a cell under conditions suitable for expression of a TGF-beta type II receptor polypeptide and a TGF-beta co-receptor polypeptide, wherein the cell comprises a first nucleic acid comprising a coding sequence for a TGF-beta type II receptor, such as those described herein, and a second nucleic acid comprising a coding sequence for the TGF-beta co-receptor, such as those described herein. In some embodiments, the disclosure relates to a method of making a heteromultimer comprising a first TGF-beta co-receptor polypeptide and a second TGF-beta co-receptor polypeptide by culturing a cell under conditions suitable for expression of a first TGF-beta co-receptor polypeptide and a second TGF-beta co-receptor polypeptide, wherein the cell comprises a first nucleic acid comprising a coding sequence for the TGF-beta co-receptor, such as those described herein and a second nucleic acid comprising a coding sequence for the TGF-beta co-receptor, such as those described herein. In some embodiments, the disclosure relates to a method of making a heteromultimer comprising a TGF-beta type I receptor polypeptide and a TGF-beta co-receptor polypeptide comprising: a) culturing a first cell under conditions suitable for expression of a TGF-beta type I receptor polypeptide, wherein the cell comprises a nucleic acid comprising a coding sequence for a TGF-beta type I receptor polypeptide; b) recovering the TGF-beta type I receptor polypeptide; c) culturing a second cell under conditions suitable for expression of a TGF-beta co-receptor polypeptide, wherein the cell comprises a nucleic acid comprising a coding sequence for a TGF-beta co-receptor polypeptide; d) recovering the TGF-beta co-receptor polypeptide; e) combining the recovered TGF-beta type I receptor polypeptide and the TGF-beta co-receptor polypeptide under conditions suitable for heteromultimer formation. In some embodiments, the disclosure relates to a method of making a heteromultimer comprising a TGF-beta type II receptor polypeptide and a TGF-beta co-receptor polypeptide comprising: a) culturing a first cell under conditions suitable for expression of a TGF-beta type II receptor polypeptide, wherein the cell comprises a nucleic acid comprising a coding sequence for a TGF-beta type II receptor polypeptide; b) recovering the TGF-beta type II receptor polypeptide; c) culturing a second cell under conditions suitable for expression of a TGF-beta co-receptor polypeptide, wherein the cell comprises a nucleic acid comprising a coding sequence for a TGF-beta co-receptor polypeptide; d) recovering the TGF-beta co-receptor polypeptide; e) combining the recovered TGF-beta type II receptor polypeptide and the TGF-beta co-receptor polypeptide under conditions suitable for heteromultimer formation. In some embodiments, the disclosure relates to a method of making a heteromultimer comprising a first TGF-beta co-receptor polypeptide and a second TGF-beta co-receptor polypeptide comprising: a) culturing a first cell under conditions suitable for expression of a first TGF-beta co-receptor polypeptide, wherein the cell comprises a nucleic acid comprising a coding sequence for a first TGF-beta co-receptor polypeptide; b) recovering the first TGF-beta co-receptor polypeptide; c) culturing a second cell under conditions suitable for expression of a second TGF-beta co-receptor polypeptide, wherein the cell comprises a nucleic acid comprising a coding sequence for a second TGF-beta co-receptor polypeptide; d) recovering the second TGF-beta co-receptor polypeptide; e) combining the recovered first TGF-beta co-receptor polypeptide and the second TGF-beta co-receptor polypeptide under conditions suitable for heteromultimer formation. Optionally, methods of making a heteromultimer as described herein may comprise a further step of recovering the heteromultimer. Heteromultimers disclosed herein may be crude, partially purified, or highly purified fractions using any of the well-known techniques for obtaining protein from cell cultures.

Any of the heteromultimers described herein may be incorporated into a pharmaceutical preparation. Optionally, such pharmaceutical preparations are at least 80%, 85%, 90%, 95%, 97%, 98% or 99% pure with respect to other polypeptide components. Optionally, pharmaceutical preparations disclosed herein may comprise one or more additional active agents. In some embodiments, heteromultimers of the disclosure comprise less than 10%, 9%, 8%, 7%, 5%, 4%, 3%, 2%, or less than 1% type I receptor polypeptide homomultimers. In some embodiments, heteromultimers of the disclosure comprise less than 10%, 9%, 8%, 7%, 5%, 4%, 3%, 2%, or less than 1% type II receptor polypeptide homomultimers. In some embodiments, heteromultimers of the disclosure comprise less than 10%, 9%, 8%, 7%, 5%, 4%, 3%, 2%, or less than 1% co-receptor polypeptide homomultimers.

The disclosure further relates to methods and heteromultimeric for use in the treatment or prevention of various disease and disorders associated with, for example, bone and red blood cells that are affected by one or more ligands of the TGF-beta superfamily. Such disease and disorders include, but are not limited to, anemia, a hemoglobinopathy, MDS, sickle-cell disease, thalassemia, and a bone-related disorder (e.g., bone-related disorders associated with low bone strength, low bone mineral density, and/or low bone growth including). In some embodiments, the disclosure relates to methods and heteromultimers for use in increasing red blood cell and/or hemoglobin levels in a patient in need thereof. In some embodiments, the disclosure relates to methods and heteromultimeric for use in increasing bone strength, bone mineral density, and/or bone growth in a patient in need thereof. In some embodiments, the disclosure relates to methods and heteromultimers for treating, preventing, and/or delaying the progression or onset of one or more complications of any one of MDS, sickle-cell disease, a thalassemia, and a hemoglobinopathy in a patient in need thereof.

### BRIEF DESCRIPTION OF THE DRAWINGS

This patent or patent application filed contains at least one drawing executed in color. Copies of this patent or patent application publication with color drawing(s) will be provided by the office upon request and payment of the necessary fee.

FIGS. 1A and 1B show two schematic examples of heteromultimer proteins comprising a TGF-beta superfamily co-receptor polypeptide and a TGF-beta superfamily type I receptor or type II receptor polypeptide. FIG. 1A depicts a heteromultimer comprising one TGF-beta superfamily co-receptor fusion polypeptide and one TGF-beta superfamily type I receptor or type II receptor fusion polypeptide, which can be assembled covalently or noncovalently via a multimerization domain contained within each polypeptide chain. Two assembled multimerization domains constitute an interaction pair, which can be either guided or unguided. FIG. 1B depicts a heteromultimer comprising two heterodimeric complexes as in FIG. 1A. Complexes of higher order can be envisioned.

FIG. 2 shows a schematic example of a heteromultimer comprising a TGF-beta superfamily co-receptor polypeptide (indicated as “co-receptor”) (e.g. a polypeptide that is at least 80%, 85%, 90%, 95%, 96%, 97%, 98%, 99% or 100% identical to an endoglin, betaglycan, Cripto-1, Cryptic protein, Cryptic family protein IB, Crim1, Crim2, BAMBI, BMPER, RGM-A, RGM-B, or hemojuvelin polypeptide from humans or other species such as those described herein, e.g., SEQ ID Nos: 501, 502, 505, 506, 509, 510, 513, 514, 517, 518, 521, 522, 525, 526, 529, 530, 533, 534, 537, 538, 541, 542, 545, 546, 549, 550, 553, 554, 557, 558, 561, 562, 565, 566, 569, 570, 573, 574, 577, 578, 581, 582, 585, 586, 589, 590, 593, or 594) and a type I receptor polypeptide (e.g. a polypeptide that is at least 80%, 85%, 90%, 95%, 96%, 97%, 98%, 99% or 100% identical to an ALK1, ALK2, ALK3, ALK4, ALK5, ALK6 or ALK7 polypeptide from humans or other species such as those described herein, e.g., SEQ ID Nos: 14, 15, 18, 19, 22, 23, 26, 27, 30, 31, 34, 35, 38, 39, 83, 84, 87, 88, 91, 92, 301, 302, 305, 306, 309, 310, and 313) or a TGF-beta superfamily type II receptor polypeptide (e.g. a polypeptide that is at least 80%, 85%, 90%, 95%, 96%, 97%, 98%, 99% or 100% identical to an ActRIIA, ActRIIB, MISRII, BMPRII, or TGFBRII polypeptide from humans or other species such as those described herein, e.g., SEQ ID Nos: 1, 2, 3, 4, 5, 6, 9, 10, 11, 42, 43, 46, 47, 50, 51, 67, 68, 71, 72, 75, 76, 79, and 80) (indicated as “I/II”). In the illustrated embodiment, the co-receptor polypeptide is part of a fusion polypeptide that comprises a first member of an interaction pair (“C”), and the type I or II receptor polypeptide is part of a fusion polypeptide that comprises a second member of an interaction pair (“D”). In each fusion polypeptide, a linker may be positioned between the co-, type I, or type II receptor polypeptide and the corresponding member of the interaction pair. The first and second members of the interaction pair (C, D) may be a guided (asymmetric) pair, meaning that the members of the pair associate preferentially with each other rather than self-associate, or the interaction pair may be unguided, meaning that the members of the pair may associate with each other or self-associate without substantial preference and may have the same or different amino acid sequences. Traditional Fc fusion proteins and antibodies are examples of unguided interaction pairs, whereas a variety of engineered Fc domains have been designed as guided (asymmetric) interaction pairs [e.g., Spiess et al (2015) Molecular Immunology 67(2A): 95-106].

FIG. 3 shows an alignment of extracellular domains of human ActRIIA (SEQ ID NO: 10) and human ActRIIB (SEQ ID NO: 2) with the residues that are deduced herein, based on composite analysis of multiple ActRIIB and ActRIIA crystal structures, to directly contact ligand indicated with boxes.

FIG. 4 shows a multiple sequence alignment of various vertebrate ActRIIB precursor proteins without their intracellular domains, human ActRIIA precursor protein without its intracellular domain, and a consensus ActRII precursor protein.

FIG. 5 shows multiple sequence alignment of Fc domains from human IgG isotypes using Clustal 2.1. Hinge regions are indicated by dotted underline. Double underline indicates examples of positions engineered in IgG1 Fc to promote asymmetric chain pairing and the corresponding positions with respect to other isotypes IgG2, IgG3 and IgG4.

FIGS. 6A-6D show schematic examples of heteromeric protein complexes comprising a TGF-beta superfamily co-receptor polypeptide (indicated as “co-receptor”) (e.g. a polypeptide that is at least 80%, 85%, 90%, 95%, 96%, 97%, 98%, 99% or 100% identical to an endoglin, betaglycan, Cripto-1, Cryptic protein, Cryptic family protein IB, Crim1, Crim2, BAMBI, BMPER, RGM-A, RGM-B, or hemojuvelin polypeptide from humans or other species such as those described herein, e.g., SEQ ID Nos: 501, 502, 505, 506, 509, 510, 513, 514, 517, 518, 521, 522, 525, 526, 529, 530, 533, 534, 537, 538, 541, 542, 545, 546, 549, 550, 553, 554, 557, 558, 561, 562, 565, 566, 569, 570, 573, 574, 577, 578, 581, 582, 585, 586, 589, 590, 593, or 594) and a type I receptor polypeptide (e.g. a polypeptide that is at least 80%, 85%, 90%, 95%, 96%, 97%, 98%, 99% or 100% identical to an ALK1, ALK2, ALK3, ALK4, ALK5, ALK6 or ALK7 polypeptide from humans or other species such as those described herein, e.g., SEQ ID Nos: 14, 15, 18, 19, 22, 23, 26, 27, 30, 31, 34, 35, 38, 39, 83, 84, 87, 88, 91, 92, 301, 302, 305, 306, 309, 310, and 313) or a TGF-beta superfamily type II receptor polypeptide (e.g. a polypeptide that is at least 80%, 85%, 90%, 95%, 96%, 97%, 98%, 99% or 100% identical to an ActRIIA, ActRIIB, MISRII, BMPRII, or TGFBRII polypeptide from humans or other species such as those described herein, e.g., SEQ ID Nos: 1, 2, 3, 4, 5, 6, 9, 10, 11, 42, 43, 46, 47, 50, 51, 67, 68, 71, 72, 75, 76, 79, and 80) (indicated as “I/II”). In the illustrated embodiments, the a co-receptor polypeptide is part of a fusion polypeptide that comprises a first member of an interaction pair (“C₁”), and a type I or type II receptor polypeptide is part of a fusion polypeptide that comprises a second member of an interaction pair (“C₂”). Suitable interaction pairs included, for example, heavy chain and/or light chain immunoglobulin interaction pairs, truncations, and variants thereof such as those described herein [e.g., Spiess et al (2015) Molecular Immunology 67(2A): 95-106]. In each fusion polypeptide, a linker may be positioned between the co-, type I, and/or type II receptor polypeptide receptor polypeptide and the corresponding member of the interaction pair. The first and second members of the interaction pair may be unguided, meaning that the members of the pair may associate with each other or self-associate without substantial preference, and they may have the same or different amino acid sequences. See FIG. 6A. Alternatively, the interaction pair may be a guided (asymmetric) pair, meaning that the members of the pair associate preferentially with each other rather than self-associate. See FIG. 6B. Complexes of higher order can be envisioned. See FIGS. 6C and 6D.

FIGS. 7A-7G show schematic examples of heteromultimers comprising two TGF-beta superfamily co-receptor polypeptides (indicated as “co-receptor”) (e.g. a polypeptide that is at least 80%, 85%, 90%, 95%, 96%, 97%, 98%, 99% or 100% identical to an endoglin, betaglycan, Cripto-1, Cryptic protein, Cryptic family protein IB, Crim1, Crim2, BAMBI, BMPER, RGM-A, RGM-B, or hemojuvelin polypeptide from humans or other species such as those described herein, e.g., SEQ ID Nos: 501, 502, 505, 506, 509, 510, 513, 514, 517, 518, 521, 522, 525, 526, 529, 530, 533, 534, 537, 538, 541, 542, 545, 546, 549, 550, 553, 554, 557, 558, 561, 562, 565, 566, 569, 570, 573, 574, 577, 578, 581, 582, 585, 586, 589, 590, 593, or 594) and two type I receptor polypeptides (e.g. a polypeptide that is at least 80%, 85%, 90%, 95%, 96%, 97%, 98%, 99% or 100% identical to an ALK1, ALK2, ALK3, ALK4, ALK5, ALK6 or ALK7 polypeptide from humans or other species such as those described herein, e.g., SEQ ID Nos: 14, 15, 18, 19, 22, 23, 26, 27, 30, 31, 34, 35, 38, 39, 83, 84, 87, 88, 91, 92, 301, 302, 305, 306, 309, 310, and 313) or TGF-beta superfamily type II receptor polypeptides (e.g. a polypeptide that is at least 80%, 85%, 90%, 95%, 96%, 97%, 98%, 99% or 100% identical to an ActRIIA, ActRIIB, MISRII, BMPRII, or TGFBRII polypeptide from humans or other species such as those described herein, e.g., SEQ ID Nos: 1, 2, 3, 4, 5, 6, 9, 10, 11, 42, 43, 46, 47, 50, 51, 67, 68, 71, 72, 75, 76, 79, and 80) (indicated as “I/II”). In the illustrated embodiment 7A, the first co-receptor polypeptide (from left to right) is part of a fusion polypeptide that comprises a first member of an interaction pair (“C₁”) and further comprises an additional first member of an interaction pair (“A₁”); and the second co-receptor polypeptide is part of a fusion polypeptide that comprises a second member of an interaction pair (“C₂”) and further comprises an first member of an interaction pair (“A₂”). The first type I or type II receptor polypeptide (from left to right) is part of a fusion polypeptide that comprises a second member of an interaction pair (“B₁”); and the second type I or type II receptor polypeptide is part of a fusion polypeptide that comprises a second member of an interaction pair (“B₂”). A₁ and A₂ may be the same or different; B₁ and B₂ may be the same or different, and C₁ and C₂ may be the same or different. In each fusion polypeptide, a linker may be positioned between the co, type I, and/or type II receptor polypeptides and the corresponding member of the interaction pair as well as between interaction pairs. FIG. 7A is an example of an association of unguided interaction pairs, meaning that the members of the pair may associate with each other or self-associate without substantial preference and may have the same or different amino acid sequences.

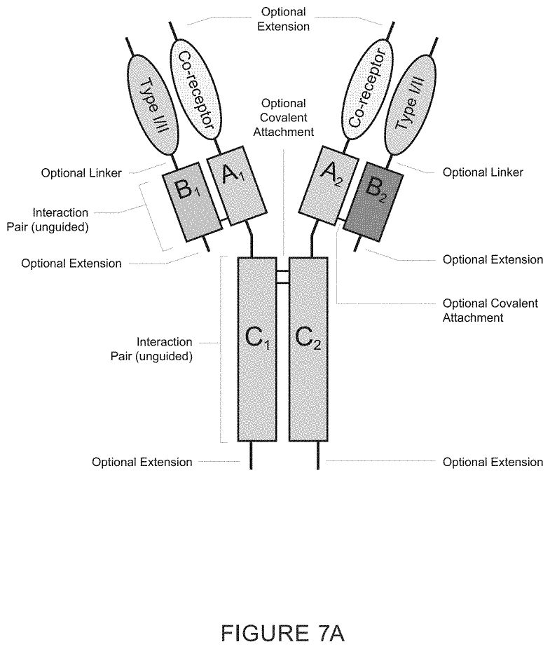

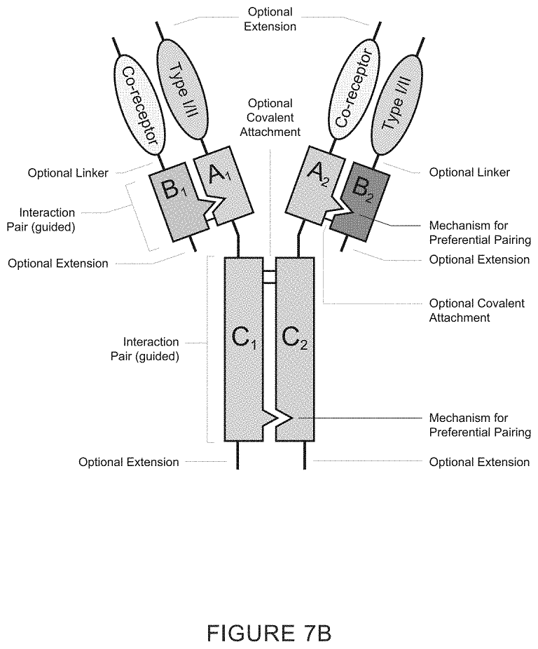

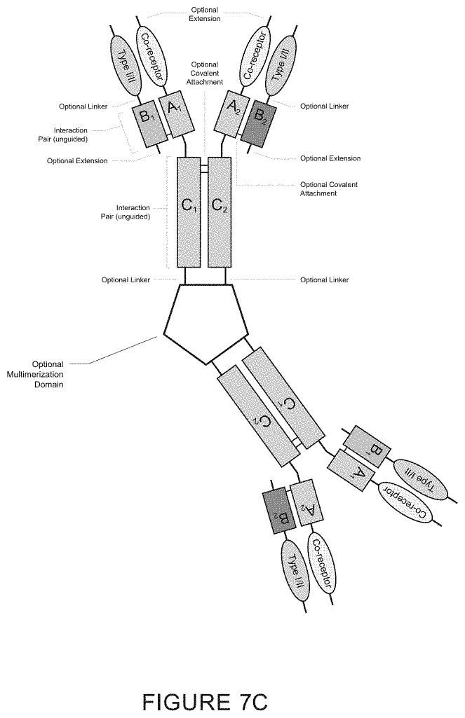

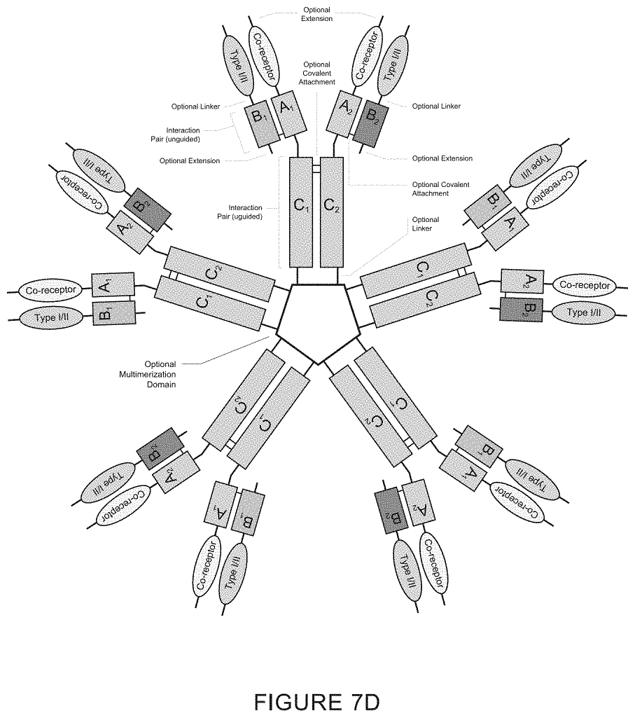

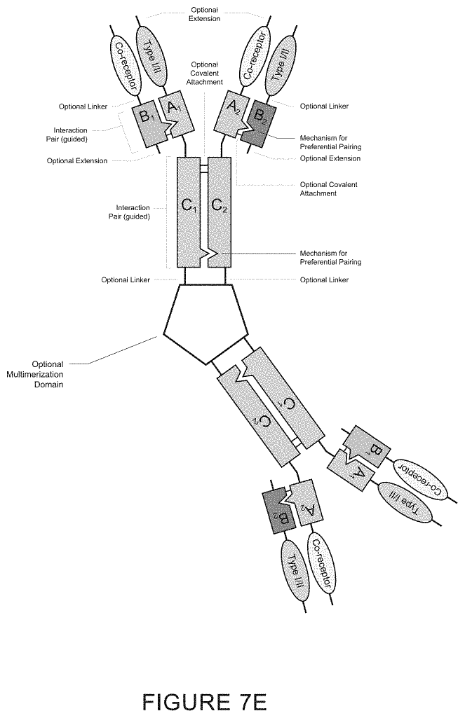

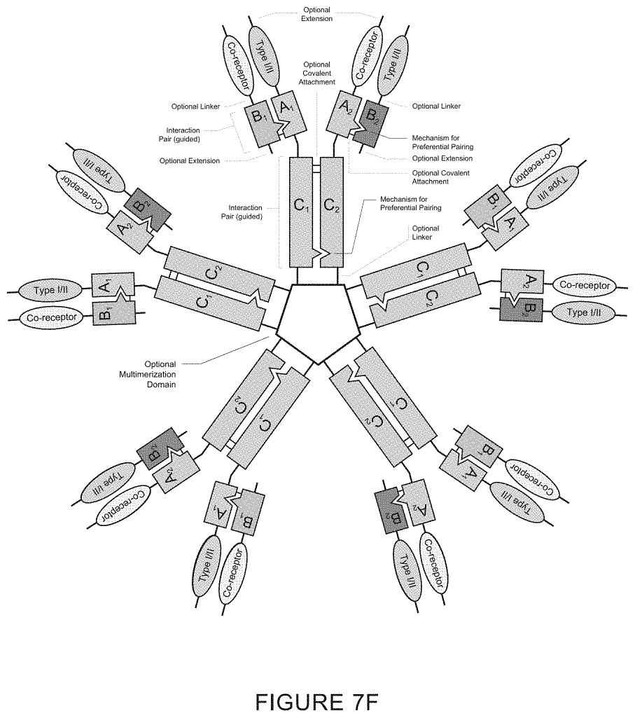

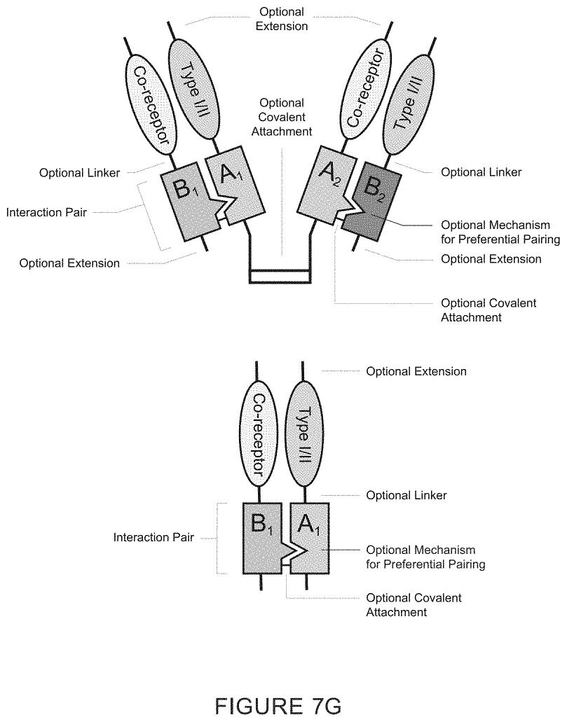

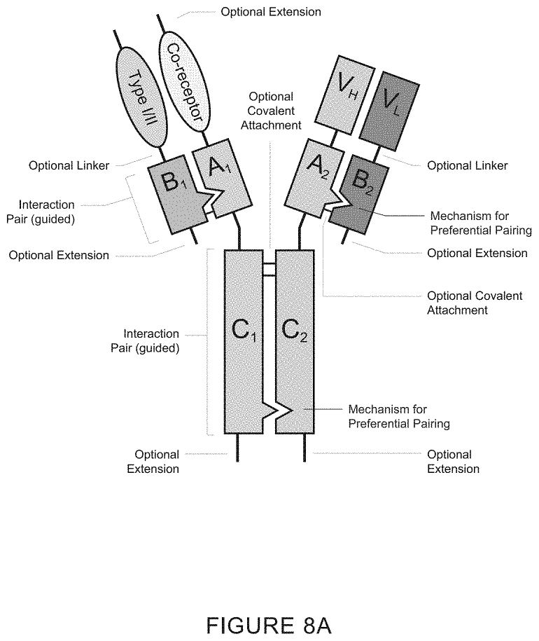

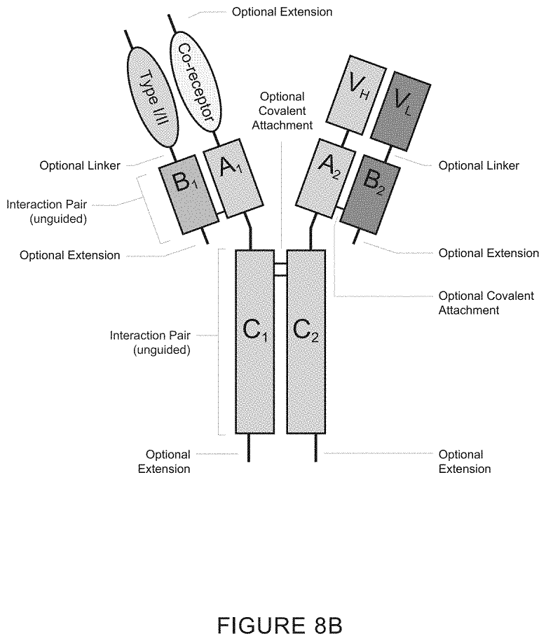

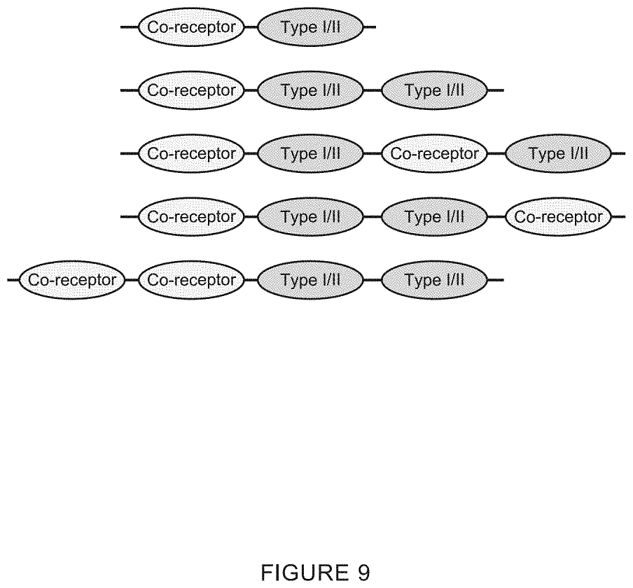

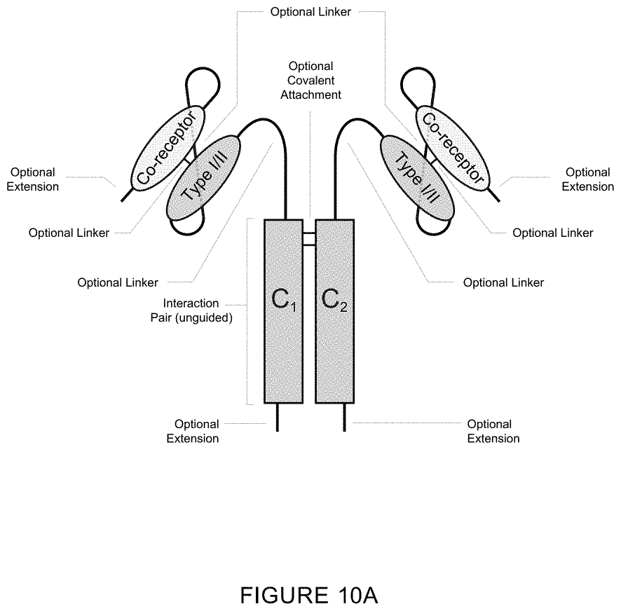

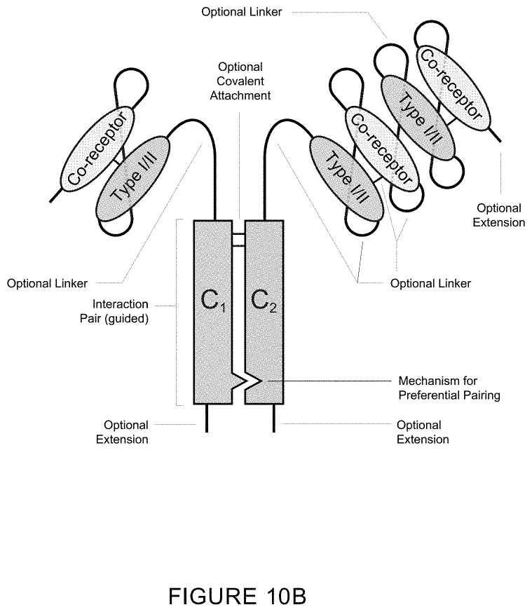

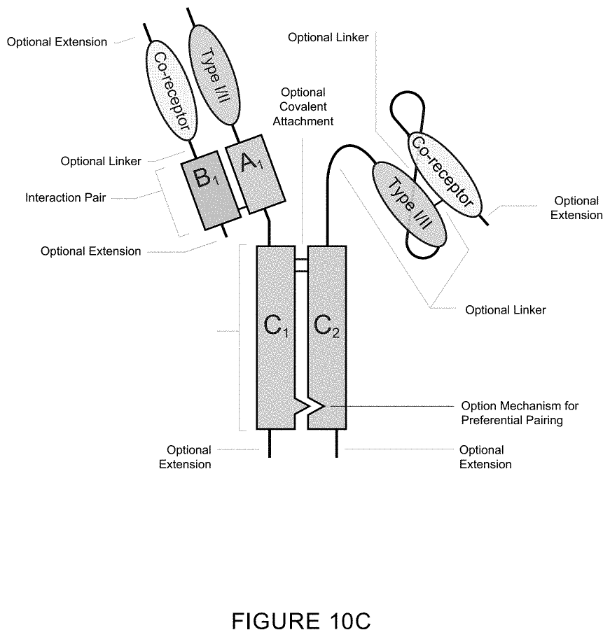

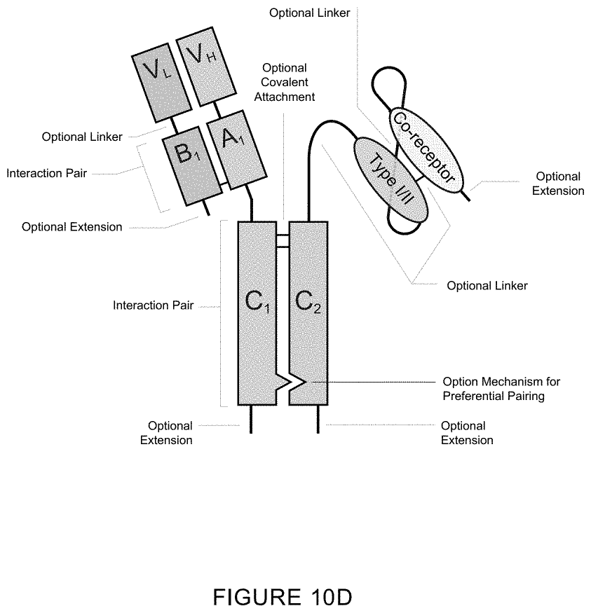

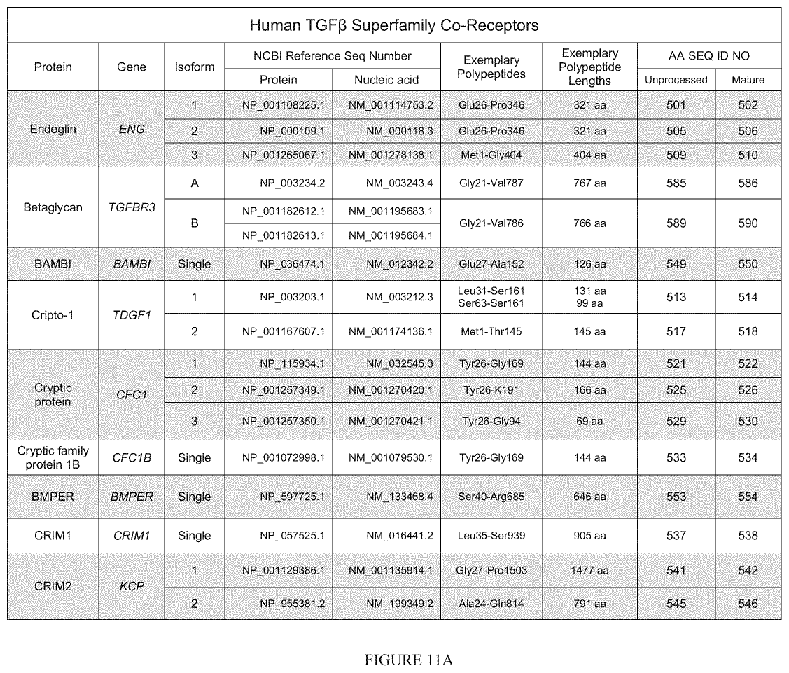

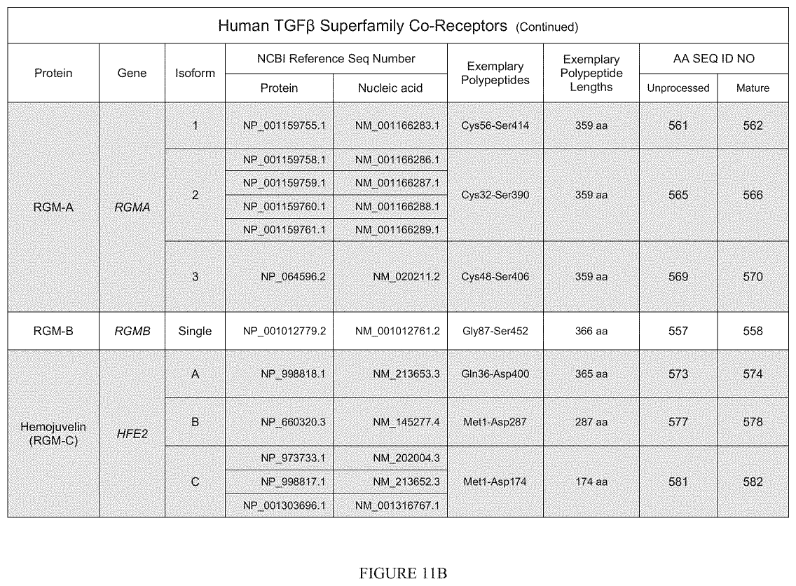

In the illustrated embodiment 7B, the first type I or type II receptor polypeptide (from left to right) is part of a fusion polypeptide that comprises a first member of an interaction pair (“C₁”) and further comprises an additional first member of an interaction pair (“A₁”); and the second type I or type II receptor polypeptide is part of a fusion polypeptide that comprises a second member of an interaction pair (“B₂”). The first co-receptor polypeptide (from left to right) is part of a fusion polypeptide that comprises a second member of an interaction pair (“B₁”); and the second co-receptor polypeptide is part of a fusion polypeptide that comprises a second member of an interaction pair (“C₂”) and further comprises a first member of an interaction pair (“A₂”). In each fusion polypeptide, a linker may be positioned between the co-, type I, and/or type II receptor polypeptide and the corresponding member of the interaction pair as well as between interaction pairs. FIG. 7B is an example of an association of guided (asymmetric) interaction pairs, meaning that the members of the pair associate preferentially with each other rather than self-associate.

Suitable interaction pairs included, for example, heavy chain and/or light chain immunoglobulin interaction pairs, truncations, and variants thereof as described herein [e.g., Spiess et al (2015) Molecular Immunology 67(2A): 95-106]. Complexes of higher order can be envisioned. See FIG. 7C-7F. Using similar methods, particularly those that employ light and/or heavy chain immunoglobulins, truncations, or variants thereof, interaction pairs may be used to produce heterodimers that resemble antibody Fab and F(ab′)₂ complexes [e.g., Spiess et al (2015) Molecular Immunology 67(2A): 95-106]. See FIG. 7G.

FIGS. 8A and 8B show schematic examples of a heteromultimers comprising a TGF-beta superfamily co-receptor (indicated as “co-receptor”) (e.g. a polypeptide that is at least 80%, 85%, 90%, 95%, 96%, 97%, 98%, 99% or 100% identical to an endoglin, betaglycan, Cripto-1, Cryptic protein, Cryptic family protein IB, Crim1, Crim2, BAMBI, BMPER, RGM-A, RGM-B, or hemojuvelin polypeptide from humans or other species such as those described herein, e.g., SEQ ID Nos: 501, 502, 505, 506, 509, 510, 513, 514, 517, 518, 521, 522, 525, 526, 529, 530, 533, 534, 537, 538, 541, 542, 545, 546, 549, 550, 553, 554, 557, 558, 561, 562, 565, 566, 569, 570, 573, 574, 577, 578, 581, 582, 585, 586, 589, 590, 593, or 594) and a type I receptor polypeptide (e.g. a polypeptide that is at least 80%, 85%, 90%, 95%, 96%, 97%, 98%, 99% or 100% identical to an ALK1, ALK2, ALK3, ALK4, ALK5, ALK6 or ALK7 polypeptide from humans or other species such as those described herein, e.g., SEQ ID Nos: 14, 15, 18, 19, 22, 23, 26, 27, 30, 31, 34, 35, 38, 39, 83, 84, 87, 88, 91, 92, 301, 302, 305, 306, 309, 310, and 313) or a TGF-beta superfamily type II receptor polypeptide (e.g. a polypeptide that is at least 80%, 85%, 90%, 95%, 96%, 97%, 98%, 99% or 100% identical to an ActRIIA, ActRIIB, MISRII, BMPRII, or TGFBRII polypeptide from humans or other species such as those described herein, e.g., SEQ ID Nos: 1, 2, 3, 4, 5, 6, 9, 10, 11, 42, 43, 46, 47, 50, 51, 67, 68, 71, 72, 75, 76, 79, and 80) (indicated as “I/II”). In the illustrated embodiments, the co-receptor polypeptide is part of a fusion polypeptide that comprises a first member of an interaction pair (“C₁”), and further comprises an additional first member of an interaction pair (“A₁”). The type I or type II receptor polypeptide is part of a fusion polypeptide that comprises a second member of an interaction pair (“B₁”). The variable heavy chain (V_{H}) polypeptide is part of a fusion polypeptide that comprises a second member of an interaction pair (“C₂”), and further comprises a first member of an interaction pair (“A₂”). The variable heavy chain (V_{L}) polypeptide is part of a fusion polypeptide that comprises a second member of an interaction pair (“B₂”). In each fusion polypeptide, a linker may be positioned between the co-, type I, and/or type II receptor polypeptide and the corresponding member of the interaction pair, between interaction pairs, and between the V_{H} and V_{L} polypeptides and a member of the interaction pair. A₁ and A₂ may be the same or different; B₁ and B₂ may be the same or different, and C₁ and C₂ may be the same or different. Suitable interaction pairs included, for example, constant heavy chain and/or light chain immunoglobulin interaction pairs, truncations, and variants thereof as described herein [e.g., Spiess et al (2015) Molecular Immunology 67(2A): 95-106]. FIG. 8A is an example of an association of guided (asymmetric) interaction pairs, meaning that the members of the pair associate preferentially with each other rather than self-associate. FIG. 8B is an example of an association of unguided interaction pairs, meaning that the members of the pair may associate with each other or self-associate without substantial preference and may have the same or different amino acid sequences.

FIG. 9 shows schematic examples of co-receptor: type I/II receptor single-trap polypeptides. Co-receptor: type I/II receptor single-trap polypeptides may contain multiple co-receptor domains (e.g., 1, 2, 3, 4, 5, 6, 7, 9, 10 or more domains), having the same or different sequences, and type I receptor domains (e.g., 1, 2, 3, 4, 5, 6, 7, 9, 10 or more domains), having the same or different sequences, or multiple type II receptor domains (e.g., 1, 2, 3, 4, 5, 6, 7, 9, 10 or more domains), having the same or different sequences. These co-receptor and type I/II receptor domains may be arranged in any order and may comprise one or more linker domains positions between one or more of the co-, type I, and/or type II receptor domains. Such ligand traps may be useful as therapeutic agents to treat or prevent diseases or disorders described herein.

FIG. 10A-10D show schematic examples of heteromultimers comprising at least one co-receptor: type I/II receptor single-chain trap polypeptide. In the illustrated embodiments 10A and 10B, a first co-receptor: type I/II receptor single-chain trap polypeptide (from left to right) is part of a fusion polypeptide that comprises a first member of an interaction pair (“C₁”); and a second co-receptor: type I/II receptor single-chain trap polypeptide is part of a fusion polypeptide that comprises a second member of an interaction pair (“C₂”). C₁ and C₂ may be the same or different. The first and second co-receptor: type I/II receptor single-chain trap polypeptides may be the same or different. In each fusion polypeptide, a linker may be positioned between the co-receptor: type I/II receptor single-chain trap polypeptide and the corresponding member of the interaction pair. Suitable interaction pairs included, for example, heavy chain and/or light chain immunoglobulin interaction pairs, truncations, and variants thereof as described herein [e.g., Spiess et al (2015) Molecular Immunology 67(2A): 95-106]. FIG. 10A is an example of an association of unguided interaction pairs, meaning that the members of the pair may associate with each other or self-associate without substantial preference and may have the same or different amino acid sequences. FIG. 10B is an example of an association of guided (asymmetric) interaction pairs, meaning that the members of the pair associate preferentially with each other rather than self-associate. Complexes of higher order can be envisioned. In addition, such co-receptor: type I/II receptor single-chain trap polypeptides may be similarly be associated, covalently or non-covalently, with one or more co-receptor, type I receptor polypeptides, and/or one or more type II receptor polypeptides. See FIG. 10C. Also, such co-receptor: type I/II receptor single-chain trap polypeptides may be similarly be associated, covalently or non-covalently, with one or more ligand-binding domain of an antibody (e.g., a ligand binding domain of an antibody that binds to one or more type I receptor: type II receptor heteromultimer binding-ligands). See FIG. 10D.

FIGS. 11A and 11B indicates exemplary polypeptides for TGFβ superfamily co-receptors, along with their amino acid (AA) and nucleotide (NT) sequence identification numbers in the present application, corresponding to individual co-receptor isoforms identified by NCBI Reference Sequence number.

### DETAILED DESCRIPTION OF THE INVENTION

### 1. Overview

In part, the disclosure provides recombinant TGF-beta superfamily heteromultimers (heteromultimers) comprising at least one TGF-beta superfamily co-receptor polypeptide (e.g., endoglin, betaglycan, Cripto-1, Cryptic, Cryptic family protein 1B, Crim1, Crim2, BAMBI, BMPER, RGM-A, RGM-B, hemojuvelin, and MuSK), including fragments and variants thereof. In some embodiments, the disclosure relates to a recombinant heteromultimer comprising a TGF-beta superfamily co-receptor polypeptide selected from the group consisting of: endoglin, betaglycan, Cripto-1, Cryptic, Cryptic family protein 1B, Crim1, Crim2, BAMBI, BMPER, RGM-A, RGM-B, hemojuvelin, and MuSK, including fragments and variants thereof, and a TGF-beta superfamily type I receptor polypeptide selected from the group consisting of: ALK1, ALK2, ALK3, ALK4, ALK5, ALK6, and ALK7, including fragments and variants thereof. In some embodiments, the disclosure relates to a recombinant heteromultimer comprising a TGF-beta superfamily co-receptor polypeptide selected from the group consisting of: endoglin, betaglycan, Cripto-1, Cryptic, Cryptic family protein 1B, Crim1, Crim2, BAMBI, BMPER, RGM-A, RGM-B, hemojuvelin, and MuSK, including fragments and variants thereof, and a TGF-beta superfamily type II receptor polypeptide selected from the group consisting of: ActRIIA, ActRIIB, TGFBRII, BMPRII, and MISRII, including fragments and variants thereof. In some embodiments, the disclosure relates to a recombinant heteromultimer comprising a first TGF-beta superfamily co-receptor polypeptide selected from the group consisting of: endoglin, betaglycan, Cripto-1, Cryptic, Cryptic family protein 1B, Crim1, Crim2, BAMBI, BMPER, RGM-A, RGM-B, hemojuvelin, and MuSK, including fragments and variants thereof, and a second TGF-beta superfamily co-receptor polypeptide selected from the group consisting of: endoglin, betaglycan, Cripto-1, Cryptic, Cryptic family protein 1B, Crim1, Crim2, BAMBI, BMPER, RGM-A, RGM-B, hemojuvelin, and MuSK, including fragments and variants thereof.

The TGF-β superfamily is comprised of over 30 secreted factors including TGF-betas, activins, nodals, bone morphogenetic proteins (BMPs), growth and differentiation factors (GDFs), and anti-Mullerian hormone (AMH). See, e.g., Weiss et al. (2013) Developmental Biology, 2(1): 47-63. Members of the superfamily, which are found in both vertebrates and invertebrates, are ubiquitously expressed in diverse tissues and function during the earliest stages of development throughout the lifetime of an animal. Indeed, TGF-β superfamily proteins are key mediators of stem cell self-renewal, gastrulation, differentiation, organ morphogenesis, and adult tissue homeostasis. Consistent with this ubiquitous activity, aberrant TGF-beta superfamily signaling is associated with a wide range of human pathologies.

Ligands of the TGF-beta superfamily share the same dimeric structure in which the central 3-1/2 turn helix of one monomer packs against the concave surface formed by the beta-strands of the other monomer. The majority of TGF-beta family members are further stabilized by an intermolecular disulfide bonds. This disulfide bond traverses through a ring formed by two other disulfide bonds generating what has been termed a ‘cysteine knot’ motif. See, e.g., Lin et al., (2006) Reproduction 132: 179-190 and Hinck et al. (2012) FEBS Letters 586: 1860-1870.

TGF-beta superfamily signaling is mediated by heteromeric complexes of type I and type II serine/threonine kinase receptors, which phosphorylate and activate downstream SMAD proteins (e.g., SMAD proteins 1, 2, 3, 5, and 8) upon ligand stimulation. See, e.g., Massague (2000) Nat. Rev. Mol. Cell Biol. 1:169-178. These type I and type II receptors are transmembrane proteins, composed of a ligand-binding extracellular domain with cysteine-rich region, a transmembrane domain, and a cytoplasmic domain with predicted serine/threonine kinase specificity. In general, type I receptors mediate intracellular signaling while the type II receptors are required for binding TGF-beta superfamily ligands. Type I and II receptors form a stable complex after ligand binding, resulting in phosphorylation of type I receptors by type II receptors.

The TGF-beta family can be divided into two phylogenetic branches based on the type I receptors they bind and the Smad proteins they activate. One is the more recently evolved branch, which includes, e.g., the TGF-betas, activins, GDF8, GDF9, GDF11, BMP3 and nodal, which signal through type I receptors that activate Smads 2 and 3 [Hinck (2012) FEBS Letters 586:1860-1870]. The other branch comprises the more distantly related proteins of the superfamily and includes, e.g., BMP2, BMP4, BMP5, BMP6, BMP7, BMP8a, BMP8b, BMP9, BMP10, GDF1, GDF5, GDF6, and GDF7, which signal through Smads 1, 5, and 8.

TGF-beta isoforms are the founding members of the TGF-beta superfamily, of which there are 3 known isoforms in mammals designated as TGF-beta1, TGF-beta2 and TGF-beta3. Mature bioactive TGF-beta ligands function as homodimers and predominantly signal through the type I receptor ALK5, but have also been found to additionally signal through ALK1 in endothelial cells. See, e.g., Goumans et al. (2003) Mol Cell 12(4): 817-828. TGF-beta1 is the most abundant and ubiquitously expressed isoform. TGF-beta1 is known to have an important role in wound healing, and mice expressing a constitutively active TGF-beta1 transgene develop fibrosis. See e.g., Clouthier et al., (1997) J Clin. Invest. 100(11): 2697-2713. TGF-beta1 is also involved in T cell activation and maintenance of T regulatory cells. See, e.g., Li et al., (2006) Immunity 25(3): 455-471. TGF-beta2 expression was first described in human glioblastoma cells, and is occurs in neurons and astroglial cells of the embryonic nervous system. TGF-beta2 is known to suppress interleukin-2-dependent growth of T lymphocytes. TGF-beta3 was initially isolated from a human rhabdomyosarcoma cell line and since has been found in carcinoma cell lines. TGF-beta3 is known to be important for palate and lung morphogenesis. See, e.g., Kubiczkova et al., (2012) Journal of Translational Medicine 10:183.

Activins are members of the TGF-beta superfamily and were initially discovered as regulators of secretion of follicle-stimulating hormone, but subsequently various reproductive and non-reproductive roles have been characterized. There are three principal activin forms (A, B, and AB) that are homo/heterodimers of two closely related β subunits (β_{A}β_{A}, β_{B}β_{B}, and β_{A}β_{B}, respectively). The human genome also encodes an activin C and an activin E, which are primarily expressed in the liver, and heterodimeric forms containing β_{C} or β_{E} are also known. In the TGF-beta superfamily, activins are unique and multifunctional factors that can stimulate hormone production in ovarian and placental cells, support neuronal cell survival, influence cell-cycle progress positively or negatively depending on cell type, and induce mesodermal differentiation at least in amphibian embryos. See, e.g., DePaolo et al. (1991) Proc Soc Ep Biol Med. 198:500-512; Dyson et al. (1997) Curr Biol. 7:81-84; and Woodruff (1998) Biochem Pharmacol. 55:953-963. In several tissues, activin signaling is antagonized by its related heterodimer, inhibin. For example, in the regulation of follicle-stimulating hormone (FSH) secretion from the pituitary, activin promotes FSH synthesis and secretion, while inhibin reduces FSH synthesis and secretion. Other proteins that may regulate activin bioactivity and/or bind to activin include follistatin (FS), follistatin-related protein (FSRP, also known as FLRG or FSTL3), and α₂-macroglobulin.

As described herein, agents that bind to “activin A” are agents that specifically bind to the β_{A} subunit, whether in the context of an isolated β_{A} subunit or as a dimeric complex (e.g., a β_{A}β_{A} homodimer or a β_{A}β_{B} heterodimer). In the case of a heterodimer complex (e.g., a β_{A}β_{B} heterodimer), agents that bind to “activin A” are specific for epitopes present within the β_{A} subunit, but do not bind to epitopes present within the non-β_{A} subunit of the complex (e.g., the 13B subunit of the complex). Similarly, agents disclosed herein that antagonize (inhibit) “activin A” are agents that inhibit one or more activities as mediated by a β_{A} subunit, whether in the context of an isolated β_{A} subunit or as a dimeric complex (e.g., a β_{A}β_{A} homodimer or a β_{A}β_{B} heterodimer). In the case of β_{A}β_{B} heterodimers, agents that inhibit “activin A” are agents that specifically inhibit one or more activities of the β_{A} subunit, but do not inhibit the activity of the non-β_{A} subunit of the complex (e.g., the β_{B} subunit of the complex). This principle applies also to agents that bind to and/or inhibit “activin B”, “activin C”, and “activin E”. Agents disclosed herein that antagonize “activin AB”, “activin AC”, “activin AE”, “activin BC”, or “activin BE” are agents that inhibit one or more activities as mediated by the β_{A} subunit and one or more activities as mediated by the β_{B} subunit. The same principle applies to agents that bind to and/or inhibit “activin AC”, “activin AE”, “activin BC”, or “activin BE”.

Nodal proteins have functions in mesoderm and endoderm induction and formation, as well as subsequent organization of axial structures such as heart and stomach in early embryogenesis. It has been demonstrated that dorsal tissue in a developing vertebrate embryo contributes predominantly to the axial structures of the notochord and pre-chordal plate while it recruits surrounding cells to form non-axial embryonic structures. Nodal appears to signal through both type I and type II receptors and intracellular effectors known as SMAD proteins. Studies support the idea that ActRIIA and ActRIIB serve as type II receptors for nodal. See, e.g., Sakuma et al. (2002) Genes Cells. 2002, 7:401-12. It is suggested that Nodal ligands interact with their co-factors (e.g., Cripto or Cryptic) to activate activin type I and type II receptors, which phosphorylate SMAD2. Nodal proteins are implicated in many events critical to the early vertebrate embryo, including mesoderm formation, anterior patterning, and left-right axis specification. Experimental evidence has demonstrated that nodal signaling activates pAR3-Lux, a luciferase reporter previously shown to respond specifically to activin and TGF-beta. However, nodal is unable to induce pTlx2-Lux, a reporter specifically responsive to bone morphogenetic proteins. Recent results provide direct biochemical evidence that nodal signaling is mediated by SMAD2 and SMAD3, which also mediate signaling by TGF-betas and activins. Further evidence has shown that the extracellular protein Cripto or Cryptic is required for nodal signaling, making it distinct from activin or TGF-beta signaling.

The BMPs and GDFs together form a family of cysteine-knot cytokines sharing the characteristic fold of the TGF-beta superfamily. See, e.g., Rider et al. (2010) Biochem J., 429(1):1-12. This family includes, for example, BMP2, BMP4, BMP6, BMP7, BMP2a, BMP3, BMP3b (also known as GDF10), BMP4, BMP5, BMP6, BMP7, BMP8, BMP8a, BMP8b, BMP9 (also known as GDF2), BMP10, BMP11 (also known as GDF11), BMP12 (also known as GDF7), BMP13 (also known as GDF6), BMP14 (also known as GDF5), BMP15, GDF1, GDF3 (also known as VGR2), GDF8 (also known as myostatin), GDF9, GDF15, and decapentaplegic. Besides the ability to induce bone formation, which gave the BMPs their name, the BMP/GDFs display morphogenetic activities in the development of a wide range of tissues. BMP/GDF homo- and hetero-dimers interact with combinations of type I and type II receptor dimers to produce multiple possible signaling complexes, leading to the activation of one of two competing sets of SMAD transcription factors. BMP/GDFs have highly specific and localized functions. These are regulated in a number of ways, including the developmental restriction of BMP/GDF expression and through the secretion of several specific BMP antagonist proteins that bind with high affinity to the cytokines. Curiously, a number of these antagonists resemble TGF-beta superfamily ligands.

Growth and differentiation factor-8 (GDF8) is also known as myostatin. GDF8 is a negative regulator of skeletal muscle mass and is highly expressed in developing and adult skeletal muscle. The GDF8 null mutation in transgenic mice is characterized by a marked hypertrophy and hyperplasia of skeletal muscle. See, e.g., McPherron et al., Nature (1997) 387:83-90. Similar increases in skeletal muscle mass are evident in naturally occurring mutations of GDF8 in cattle and, strikingly, in humans. See, e.g., Ashmore et al. (1974) Growth, 38:501-507; Swatland and Kieffer, J. Anim. Sci. (1994) 38:752-757; McPherron and Lee, Proc. Natl. Acad. Sci. USA (1997) 94:12457-12461; Kambadur et al., Genome Res. (1997) 7:910-915; and Schuelke et al. (2004) N Engl J Med, 350:2682-8. Studies have also shown that muscle wasting associated with HIV-infection in humans is accompanied by increases in GDF8 protein expression. See, e.g., Gonzalez-Cadavid et al., PNAS (1998) 95:14938-43. In addition, GDF8 can modulate the production of muscle-specific enzymes (e.g., creatine kinase) and modulate myoblast cell proliferation. See, e.g., International Patent Application Publication No. WO 00/43781). The GDF8 propeptide can noncovalently bind to the mature GDF8 domain dimer, inactivating its biological activity. See, e.g., Miyazono et al. (1988) J. Biol. Chem., 263: 6407-6415; Wakefield et al. (1988) J. Biol. Chem., 263; 7646-7654; and Brown et al. (1990) Growth Factors, 3: 35-43. Other proteins which bind to GDF8 or structurally related proteins and inhibit their biological activity include follistatin, and potentially, follistatin-related proteins. See, e.g., Gamer et al. (1999) Dev. Biol., 208: 222-232.

GDF11, also known as BMP11, is a secreted protein that is expressed in the tail bud, limb bud, maxillary and mandibular arches, and dorsal root ganglia during mouse development. See, e.g., McPherron et al. (1999) Nat. Genet., 22: 260-264; and Nakashima et al. (1999) Mech. Dev., 80: 185-189. GDF11 plays a unique role in patterning both mesodermal and neural tissues. See, e.g., Gamer et al. (1999) Dev Biol., 208:222-32. GDF11 was shown to be a negative regulator of chondrogenesis and myogenesis in developing chick limb. See, e.g., Gamer et al. (2001) Dev Biol., 229:407-20. The expression of GDF11 in muscle also suggests its role in regulating muscle growth in a similar way to GDF8. In addition, the expression of GDF11 in brain suggests that GDF11 may also possess activities that relate to the function of the nervous system. Interestingly, GDF11 was found to inhibit neurogenesis in the olfactory epithelium. See, e.g., Wu et al. (2003) Neuron., 37:197-207. Hence, GDF11 may have in vitro and in vivo applications in the treatment of diseases such as muscle diseases and neurodegenerative diseases (e.g., amyotrophic lateral sclerosis).

BMP7, also called osteogenic protein-1 (OP-1), is well known to induce cartilage and bone formation. In addition, BMP7 regulates a wide array of physiological processes. For example, BMP7 may be the osteoinductive factor responsible for the phenomenon of epithelial osteogenesis. It is also found that BMP7 plays a role in calcium regulation and bone homeostasis. Like activin, BMP7 binds to type II receptors, ActRIIA and ActRIIB. However, BMP7 and activin recruit distinct type I receptors into heteromeric receptor complexes. The major BMP7 type I receptor observed was ALK2, while activin bound exclusively to ALK4 (ActRIIB). BMP7 and activin elicited distinct biological responses and activated different SMAD pathways. See, e.g., Macias-Silva et al. (1998) J Biol Chem. 273:25628-36.

Anti-Mullerian hormone (AMH), also known as Mullerian-inhibiting substance (MIS), is a TGF-beta family glycoprotein. One AMH-associated type II receptor has been identified and is designated as AMHRII, or alternatively MISRII. AMH induces regression of the Mullerian ducts in the human male embryo. AMH is expressed in reproductive age women and does not fluctuate with cycle or pregnancy, but was found to gradual decrease as both oocyte quantity and quality decrease, suggesting AMH could serve as a biomarker for ovarian physiology. See e.g. Zec et al., (2011) Biochemia Medica 21(3): 219-30.

Activin receptor-like kinase-1 (ALK1), the product of the ACVRL1 gene known alternatively as ACVRLK1, is a type I receptor whose expression is predominantly restricted to endothelial cells. See, e.g., OMIM entry 601284. ALK1 is activated by the binding of TGF-beta family ligands such as BMP9 and BMP10, and ALK1 signaling is critical in the regulation of both developmental and pathological blood vessel formation. ALK1 expression overlaps with sites of vasculogenesis and angiogenesis in early mouse development, and ALK1 knockout mice die around embryonic day 11.5 because of severe vascular abnormalities (see e.g., Cunha and Pietras (2011) Blood 117(26):6999-7006.) ALK1 expression has also been described in other cell types such as hepatic stellate cells and chondrocytes. Additionally, ALK1 along with activin receptor-like kinase-2 (ALK2) have been found to be important for BMP9-induced osteogenic signaling in mesenchymal stem cells. See e.g., Cunha and Pietras (2011) Blood 117(26):6999-7006.

ALK2, the product of the ACVR1 gene known alternatively as ActRIA or ACVRLK2, is a type I receptor that has been shown to bind activins and BMPs. ALK2 is critical for embryogenesis as ALK2 knockout mice die soon after gastrulation. See, e.g., Mishina et al. (1999) Dev Biol. 213: 314-326 and OMIM entry 102576. Constitutively active mutations in ALK2 are associated with fibrodysplasia ossificans progressiva (FOP). FOP is rare genetic disorder that causes fibrous tissue, including muscle, tendon and ligament, to be ossified spontaneously or when damaged. An arginine to histidine mutation in codon 206 of ALK2 is naturally occurring mutation associated with FOP in humans. This mutation induces BMP-specific signaling via ALK2 without the binding of ligand. See, e.g., Fukuda et al., (2009) J Biol Chem. 284(11):7149-7156 and Kaplan et al., (2011) Ann N.Y. Acad Sci. 1237: 5-10.

Activin receptor-like kinase-3 (ALK3), the product of the BMPR1A gene known alternatively as ACVRLK3, is a type I receptor mediating effects of multiple ligands in the BMP family. Unlike several type I receptors with ubiquitous tissue expression, ALK3 displays a restricted pattern of expression consistent with more specialized functionality. See, e.g., ten Dijke (1993) Oncogene, 8: 2879-2887 and OMIM entry 601299. ALK3 is generally recognized as a high affinity receptor for BMP2, BMP4, BMP7 and other members of the BMP family. BMP2 and BMP7 are potent stimulators of osteoblastic differentiation, and are now used clinically to induce bone formation in spine fusions and certain non-union fractures. ALK3 is regarded as a key receptor in mediating BMP2 and BMP4 signaling in osteoblasts. See, e.g., Lavery et al. (2008) J. Biol. Chem. 283: 20948-20958. A homozygous ALK3 knockout mouse dies early in embryogenesis (day 9.5), however, adult mice carrying a conditional disruption of ALK3 in osteoblasts have been recently reported to exhibit increased bone mass, although the newly formed bone showed evidence of disorganization. See, e.g., Kamiya (2008) J. Bone Miner. Res., 23:2007-2017; and Kamiya (2008) Development 135: 3801-3811. This finding is in startling contrast to the effectiveness of BMP2 and BMP7 (ligands for ALK3) as bone building agents in clinical use.

Activin receptor-like kinase-4 (ALK4), the product of the ACVR1B gene alternatively known as ACVRLK4, is a type I receptor that transduces signaling for a number of TGF-beta family ligands including activins, nodal and GDFs. ALK4 mutations are associated with pancreatic cancer and expression of dominant negative truncated ALK4 isoforms are highly expressed in human pituitary tumors. See, e.g., Tsuchida et al., (2008) Endocrine Journal 55(1):11-21 and OMIM entry 601300.

Activin receptor-like kinase-5 (ALK5), the product of the TGFBR1 gene, is widely expressed in most cell types. Several TGF-beta superfamily ligands, including TGF-betas, activin, and GDF-8, signal via ALK5 and activate downstream Smad 2 and Smad 3. Mice deficient in ALK5 exhibit severe defects in the vascular development of the yolk sac and placenta, lack circulating red blood cells, and die mid-gestation. It was found that these embryos had normal hematopoietic potential, but enhanced proliferation and improper migration of endothelial cells. Thus, ALK5-dependent signaling is important for angiogenesis, but not for the development of hematopoietic progenitor cells and functional hematopoiesis. See, e.g. Larsson et al., (2001) The EMBO Journal, 20(7): 1663-1673 and OMIM entry 190181. In endothelial cells, ALK5 acts cooperatively and opposite to ALK1 signaling. ALK5 inhibits cell migration and proliferation, notably the opposite effect of ALK1. See, e.g., Goumans et al. (2003) Mol Cell 12(4): 817-828. Additionally, ALK5 is believed to negatively regulate muscle growth. Knockdown of ALK5 in the muscle a mouse model of muscular dystrophy was found to decrease fibrosis and increase expression of genes associate with muscle growth. See, e.g. Kemaladewi et al., (2014) Mol Ther Nucleic Acids 3, e156.

Activin receptor-like kinase-6 (ALK6) is the product of the BMPR1B gene, whose deficiency is associated with chrondodysplasia and limb defects in both humans and mice. See, e.g., Demirhan et al., (2005) J Med Genet. 42:314-317. ALK6 is widely expressed throughout the developing skeleton, and is required for chondrogenesis in mice. See, e.g., Yi et al., (2000) Development 127:621-630 and OMIM entry 603248.

Activin receptor-like kinase-7 (ALK7) is the product of the ACVR1C gene. ALK7 null mice are viable, fertile, and display no skeletal or limb malformations. GDF3 signaling through ALK7 appears to play a role in insulin sensitivity and obesity. This is supported by results that Alk7 null mice show reduced fat accumulation and resistance to diet-induced obesity. See, e.g., Andersson et al., (2008) PNAS 105(20): 7252-7256. ALK7-mediated Nodal signaling has been implicated to have both tumor promoting and tumor suppressing effects in a variety of different cancer cell lines. See, e.g., De Silva et al., (2012) Frontiers in Endocrinology 3:59 and OMIM entry 608981.

As used herein the term “ActRII” refers to the family of type II activin receptors. This family includes both the activin receptor type IIA (ActRIIA), encoded by the ACVR2A gene, and the activin receptor type IIB (ActRIIB), encoded by the ACVR2B gene. ActRII receptors are TGF-beta superfamily type II receptors that bind a variety of TGF-beta superfamily ligands including activins, GDF8 (myostatin), GDF11, and a subset of BMPs, notably BMP6 and BMP7. ActRII receptors are implicated in a variety of biological disorders including muscle and neuromuscular disorders (e.g., muscular dystrophy, amyotrophic lateral sclerosis (ALS), and muscle atrophy), undesired bone/cartilage growth, adipose tissue disorders (e.g., obesity), metabolic disorders (e.g., type 2 diabetes), and neurodegenerative disorders. See, e.g., Tsuchida et al., (2008) Endocrine Journal 55(1):11-21, Knopf et al., U.S. Pat. No. 8,252,900, and OMIM entries 102581 and 602730.

Transforming growth factor beta receptor II (TGFBRII), encoded by the TGFBR2 gene, is a type II receptor that is known to bind TGF-beta ligands and activate downstream Smad 2 and Smad 3 effectors. See, e.g., Hinck (2012) FEBS Letters 586: 1860-1870 and OMIM entry 190182. TGF-beta signaling through TGFBRII is critical in T-cell proliferation, maintenance of T regulatory cells and proliferation of precartilaginous stem cells. See, e.g., Li et al., (2006) Immunity 25(3): 455-471 and Cheng et al., Int. J. Mol. Sci. 2014, 15, 12665-12676.

Bone morphogenetic protein receptor II (BMPRII), encoded by the BMPR2 gene, is a type II receptor that is thought to bind certain BMP ligands. In some instances, efficient ligand binding to BMPRII is dependent on the presence of the appropriate TGFBR type I receptors. See, e.g., Rosenzweig et al., (1995) PNAS 92:7632-7636. Mutations in BMPRII are associated pulmonary hypertension in humans. See OMIM entry 600799.

Müllerian-inhibiting substance receptor II (MISRII), the product of the AMHR2 gene known alternatively as anti-Müllerian hormone type II receptor, is a type II TGF-beta receptor. MISRII binds the MIS ligand, but requires the presence of an appropriate type I receptor, such as ALK3 or ALK6, for signal transduction. See, e.g., Hinck (2012) FEBS Letters 586:1860-1870 and OMIM entry 600956. MISRII is involved in sex differentiation in humans and is required for Müllerian regression in the human male. AMH is expressed in reproductive age women and does not fluctuate with cycle or pregnancy, but was found to gradual decrease as both oocyte quantity and quality decrease, suggesting AMH could serve as a biomarker of ovarian physiology. See, e.g., Zec et al., (2011) Biochemia Medica 21(3): 219-30 and OMIM entry 600956.

In certain aspects, the present invention relates to ENG polypeptides. The protein endoglin (ENG), also known as CD105 and encoded by ENG, is considered a co-receptor for the transforming growth factor-β (TGF-β) superfamily of ligands and is implicated in normal and pathological fibrosis and angiogenesis. Structurally, ENG is a homodimeric cell-surface glycoprotein. It belongs to the zona pellucida (ZP) family of proteins and consists of a short C-terminal cytoplasmic domain, a single hydrophobic transmembrane domain, and a long extracellular domain (ECD) (Gougos et al, 1990, J Biol Chem 265:8361-8364). As determined by electron microscopy, monomeric ENG ECD consists of two ZP regions and an orphan domain located at the N-terminus (Llorca et al, 2007, J Mol Biol 365:694-705).

ENG expression is low in quiescent vascular endothelium but upregulated in endothelial cells of healing wounds, developing embryos, inflammatory tissues, and solid tumors (Dallas et al, 2008, Clin Cancer Res 14:1931-1937). Mice homozygous for null ENG alleles die early in gestation due to defective vascular development (Li et al, 1999, Science 284:1534-1537), whereas heterozygous null ENG mice display angiogenic abnormalities as adults (Jerkic et al, 2006, Cardiovasc Res 69:845-854). In humans, ENG gene mutations have been identified as the cause of hereditary hemorrhagic telangiectasia (Osler-Rendu-Weber syndrome) type-1 (HHT-1), an autosomal dominant form of vascular dysplasia characterized by arteriovenous malformations resulting in direct flow (communication) from artery to vein (arteriovenous shunt) without an intervening capillary bed (McAllister et al, 1994, Nat Genet 8:345-351; Fernandez-L et al, 2006, Clin Med Res 4:66-78). Typical symptoms of patients with HHT include recurrent epistaxis, gastrointestinal hemorrhage, cutaneous and mucocutaneous telangiectases, and arteriovenous malformations in the pulmonary, cerebral, or hepatic vasculature.

As a co-receptor, ENG is thought to modulate responses of other receptors to TGF-β family ligands without direct mediation of ligand signaling by itself. Ligands in the TGF-β family typically signal by binding to a homodimeric type II receptor, which triggers recruitment and transphosphorylation of a homodimeric type I receptor, thereby leading to phosphorylation of Smad proteins responsible for transcriptional activation of specific genes (Massague, 2000, Nat Rev Mol Cell Biol 1:169-178). Based on ectopic cellular expression assays, it has been reported that ENG cannot bind ligands on its own and that its binding to TGF-β1, TGF-β3, activin A, bone morphogenetic protein-2 (BMP-2), and BMP-7 requires the presence of an appropriate type I and/or type II receptor (Barbara et al, 1999, J Biol Chem 274:584-594). Nevertheless, there is evidence that ENG expressed by a fibroblast cell line can bind TGF-β1 (St.-Jacques et al, 1994, Endocrinology 134:2645-2657), and recent results in COS cells indicate that transfected full-length ENG can bind BMP-9 in the absence of transfected type I or type II receptors (Scharpfenecker et al, 2007, J Cell Sci 120:964-972).

In addition to the foregoing, ENG can occur in a soluble form in vivo under certain conditions after proteolytic cleavage of the full-length membrane-bound protein (Hawinkels et al, 2010, Cancer Res 70:4141-4150). Elevated levels of soluble ENG have been observed in the circulation of patients with cancer and preeclampsia (Li et al, 2000, Int J Cancer 89:122-126; Calabro et al, 2003, J Cell Physiol 194:171-175; Venkatesha et al, 2006, Nat Med 12:642-649; Levine et al, 2006, N Engl J Med 355:992-1005). Although the role of endogenous soluble ENG is poorly understood, a protein corresponding to residues 26-437 of the ENG precursor (amino acids 26-437 of SEQ ID NO: 1) has been proposed to act as a scavenger or trap for TGF-β family ligands (Venkatesha et al, 2006, Nat Med 12:642-649; WO-2007/143023), of which only TGF-β1 and TGF-β3 have specifically been implicated.

In certain aspects, the present invention relates to betaglycan polypeptides. Betaglycan, also known as TGFβ receptor type III (TβRIII, TGFβRIII) and encoded by TGFBR3, is a single-pass transmembrane protein consisting of a large extracellular domain, transmembrane domain, and relatively short cytoplasmic domain (43 amino acids). It is thought that betaglycan is not directly involved in signal transduction since its cytoplasmic domain lacks an obvious signaling motif. Consistent with a co-receptor role, the presence of betaglycan on the cell surface increases the binding of TGFβ isoforms to their type II receptor (TGFβRII) and increases ligand efficacy in biologic assays (Bilandzic et al., 2011, Mol Cell Endocrinol 339:180-189). This effect is most pronounced for TGFβ2, which binds weakly to TGFβRII in the absence of betaglycan (Lopez-Casillas et al., 1993, 1994). In addition, the extracellular domain of betaglycan is released from some cells in a soluble form whose physiologic role remains to be determined.

Betaglycan can alter signaling by superfamily ligands besides TGFβ. For example, inhibin is capable of binding ActRIIA or ActRIIB and functionally antagonizing activins by preventing recruitment of activin type I receptors. However, inhibin requires the presence of betaglycan for high potency inhibition of activin signaling (Lewis et al., 2000, Nature 404:411-414; Wiater et al., 2009, Mol Endocrinol 23:1033-1042). Betaglycan forms a stable complex with inhibin and activin type II receptors, thus reducing the availability of these receptors to transmit activin signaling (Lewis et al., 2000, Nature 404:411-414). In a similar manner, betaglycan enables inhibin to antagonize the binding of BMPs to ActRIIA, ActRIIB, or BMPRII, thereby inhibiting BMP signaling (Wiater et al., 2003, J Biol Chem 278:7934-7941).

In certain aspects, the present invention relates to EGF-CFC family polypeptides. Members of the epidermal growth factor-Cripto-1/FRL-1/Cryptic (EGF-CFC) family in humans include founder Cripto-1 (encoded by TDGF1) as well as Cryptic protein (encoded by CFC1) and Cryptic family protein 1B (encoded by CFC1B). EGF-CFC genes encode small extracellular proteins that contain a divergent EGF motif and a novel conserved cysteine-rich domain termed the CFC motif, with most sequence similarity occurring in the central EGF and CFC motifs (Shen et al., 2000, Trends Genet 16:303-309). Most EGF-CFC proteins have been shown or predicted to possess a glycosylphosphatidylinositol (GPI) anchor site at the C-terminus. However, soluble extracellular forms of these proteins also exist (see, e.g., Watanabe et al., 2007, J Biol Chem 282:31643-31655).

In certain aspects, the present invention relates to Cripto-1 polypeptides. Cripto-1, also known as Cripto or teratocarcinoma-derived growth factor (TDGF-1), regulates the activity of multiple TGFβ superfamily ligands that signal via the Smad2/3 pathway. Cripto-1 functions as an obligatory cell-surface co-receptor for a subset of ligands including Nodal, GDF1, and GDF3 (Gray et al., 2012, FEBS Lett 586:1836-1845). Cripto-1 acts as a co-receptor for Nodal by recruiting ALK4, leading to formation of an ActRIIB-ALK4-Cripto-Nodal complex for signaling (Rosa, 2002, Sci STKE 2002(158):pe47; Yan et al., 2002, Mol Cell Biol 22:4439-4449; Blanchet et al., 2008, Sci Signal 1(45):ra13). This co-receptor function plays essential roles in regulating stem cell differentiation and vertebrate embryogenesis and regulates normal tissue growth and remodeling in adult tissues. See, e.g., Guardiola et al. (2012) Proc Natl Acad Sci USA 109:E3231-E3240. Cripto-1 co-receptor function has also been linked to tumor growth since Nodal signaling plays a key role in promoting tumorigenicity. In addition to facilitating signaling by some ligands, Cripto-1 inhibits receptor activation by activin A, activin B, myostatin (GDF8), and TGFβ (Gray et al., 2003, Proc Natl Acad Sci USA 100:5193-5198; Gray et al., 2006, Mol Cell Biol 26:9268-9278; Guardiola et al., 2012, Proc Natl Acad Sci USA 109:E3231-E3240). It has been shown in a detailed analysis that Cripto-1 forms analogous receptor complexes with Nodal and activin and thereby functions as a noncompetitive activin antagonist (Kelber et al., 2008, J Biol Chem 283:4490-4500).

In certain aspects, the present invention relates to Cryptic and Cryptic family 1B polypeptides. On the basis of phenotypes in double null mutant mice, Cryptic and Cripto-1 have been found to serve partially redundant functions during early embryonic development, and most if not all Nodal activity in early mouse embryogenesis is thought to be dependent on these two EGF-CFC proteins (Chu et al., 2010, Dev Biol 342:63-73). A separate study of mice deficient only in Cryptic has revealed a role for this protein in correct establishment of left-right asymmetry during embryogenesis (Gaio et al., 1999, Curr Biol 9:1339-1342).

In certain aspects, the present invention relates to chordin-related polypeptides. Proteins in this family contain chordin-like cysteine-rich repeat (CRR) motifs of the von Willebrand C (VWC) type which are important for protein binding to superfamily ligands. Such CRRs have a conserved consensus sequence based on ten cysteines (CXₙWX₄CX₂CXCX₆CX₄CX₄₋₆CX₉₋₁₁CCPXC) (Sasai et al., 1994, Cell 79:779-790; Garcia-Abreu et al., 2002, Gene 287:39-47). Examples of chordin-related proteins include BMPER, CRIM1, and CRIM2.

In certain aspects, the present invention relates to BMPER polypeptides. BMP-binding endothelial cell precursor-derived regulator (BMPER) is encoded by BMPER and is the human homolog of Drosophila Crossveinless-2 (CV-2). BMPER is a secreted protein containing five CCR motifs and is reported to be proteolytically cleaved to generate two fragments that are disulfide-linked (Moser et al., 2003, Mol Cell Biol 23:5664-5679; Binnerts et al., 2004, Biochem Biophys Res Commun 315:272-280). Mammalian BMPER was originally identified as an inhibitor of BMP signaling. However, subsequent investigation determined that BMPER can exert biphasic activity depending on concentration, enhancing BMP-mediated signaling at molar concentrations less than that of ligand but inhibiting such signaling at concentrations exceeding those of ligand (Kelley et al., 2009, J Cell Biol 184:597-609). BMPER is implicated in a wide range of BMP-mediated differentiation processes during embryonic development and also implicated as an important postnatal regulator of BMP-mediated vascular inflammation in mice (Pi et al., 2012, Arterioscler Thromb Vasc Biol 32:2214-2222).

In certain aspects, the present invention relates to CRIM1 polypeptides. Cysteine-rich motor neuron 1 (CRIM1), also known as “cysteine-rich transmembrane BMP regulator 1”, is encoded by CRIM1. This type I transmembrane protein contains a signal sequence, an extracellular domain (905 amino acids), a transmembrane domain (21 amino acids), and an intracellular domain (76 amino acids). The extracellular domain can also be released from the cell as a soluble form, likely via cleavage of the full protein at the membrane (Wilkinson et al., 2003, J Biol Chem 278:34181-34188), and contains an N-terminal insulin-like growth factor-binding motif and six chordin-like CRR motifs of the VWC type. These CRRs mediate protein binding to superfamily ligands such as TGFβ isoforms, BMP4, and BMP7 (see, e.g., Wilkinson et al., 2003, J Biol Chem 278:34181-34188). CRIM1 inhibits BMP signaling in part by reducing the rate of processing and delivery of BMPs to the cell surface. Studies in transgenic mice expressing a dominant negative (truncated) CRIM1 isoform indicate the importance of CRIM1 for normal development of the eye, central nervous system, and kidney (Pennisi et al., 2007, Dev Dyn 236:502-511; Wilkinson et al., 2007, J Am Soc Nephrol 18:1697-1708).

In certain aspects, the present invention relates to CRIM2 polypeptides. CRIM2 is a secreted protein encoded by the human gene KCP (kielin/chordin-like protein 1), named in recognition of the protein's sequence similarity to Xenopus kielin and mouse chordin. The longest CRIM2 isoform, which is nearly 1500 amino acids in human, contains many CRR motifs of the VWC type. Unlike most inhibitory proteins containing CRR motifs, CRIM2 is a potent enhancer of BMP signaling and is able to increase the affinity of BMP7 for its type I receptor ALK3 and/or enhance the stability of this ligand-receptor complex in mice (Lin et al., 2005, Nat Med 11:387-393). Mice homozygous for a CRIM2 null allele are viable and fertile but are hypersensitive to developing renal interstitial fibrosis, a disease stimulated by TGFβ but inhibited by BMP7. In contrast to the enhancing effect on BMPs, CRIM2 inhibits both activin A-mediated and TGFβ1-mediated signaling through the Smad2/3 pathway (Lin et al., 2006, Mol Cell Biol 26:4577-4585). These inhibitory effects of CRIM2 are mediated in a paracrine manner, suggesting that direct binding of CRIM2 to TGFβ1 or activin A can block interactions of these ligands with prospective receptors. The ability to enhance BMP signaling while suppressing activation by TGFβ and activin indicates an important role for CRIM2 in modulating responses between these antifibrotic and profibrotic cytokines in the initiation and progression of renal interstitial fibrosis.

In certain aspects, the present invention relates to BAMBI polypeptides. The protein named “BMP and activin membrane-bound inhibitor” (BAMBI), also known as “non-metastatic gene A” (NMA), is encoded by BAMBI. BAMBI resembles a type I receptor from the TGFβ superfamily, with an extracellular domain (132 amino acids), a transmembrane domain, and a cytoplasmic domain. However, BAMBI lacks an intracellular kinase domain and has therefore been described as a pseudoreceptor (Onichtchouk et al., 1999, Nature 401:480-485). BAMBI competes with type I receptors to form stable complexes with type II receptors and thereby prevents the formation of active complexes of type I and type II receptors. Additionally, BAMBI cooperates with Smad7 to inhibit ligand-mediated signaling (Yan et al., 2009, J Biol Chem 284:30097-30104). Ligands inhibited by BAMBI include BMPs, activin, and TGFβ. During development, BAMBI is prominent in gastrulation, neurulation, and development of bones and teeth, and is often co-expressed with BMP family members (Onichtchouk et al., 1999, Nature 401:480-485; Knight et al., J Dent Res 80:1895; Paulsen et al., 2011, Proc Natl Acad Sci USA 108:10202-). In the adult, BAMBI modulates processes such as diabetic nephropathy, thrombus formation, response to cardiac overload, and TGFβ-mediated tumor invasiveness (Villar et al., 2013, Biochim Biophys Acta 1832:323-335; Salles-Crawley et al., 2014, Blood 123:2873-2881; Fan et al., 2015, Diabetes 64:2220-2233; Marwitz et al., 2016, Cancer Res 76:3785-3801).

In certain aspects, the present invention relates to repulsive guidance molecule (RGM) polypeptides. RGMs constitute a family of structurally related proteins that have been proposed to act as co-receptors for BMP signaling and also interact with an unrelated transmembrane protein known as neogenin. The three mammalian proteins, RGM-A, RGM-B, and RGM-C, are approximately 50-60% identical in primary amino acid sequence and share structural features such as a proteolytic cleavage site and GPI anchor but undergo distinct biosynthetic and processing steps. Each RGM exhibits a distinct tissue-specific pattern of gene expression (Oldecamp et al., 2004, Gene Expr Patterns 4:283-288) and is thought to serve distinct biologic functions (see below). Soluble RGM proteins, which could form by shedding (Lin et al., 2008, Blood Cells Mol Dis 40:122-131; Tassew et al., 2012, Dev Cell 22:391-402), have been shown to inhibit BMP activity (Lin et al., 2005, Blood 106:2884-2889). A recent structural study reveals that the N-terminal domains of RGMs mimic a key BMP-binding motif of type I superfamily receptors, which could enable membrane-anchored RGMs to compete with type I receptors for BMP binding in a pH-dependent manner and yet eventually enhance BMP signaling from within an endosomal compartment (Healey et al., 2015, Nat Struct Mol Biol 22:458-465; Mueller, 2015, Nat Struct Mol Biol 22:439-440). As determined by surface plasmon resonance, the three RGM proteins exhibit differential binding kinetics for BMPs, which may contribute to their context-specific effects in vivo (Wu et al., 2012, PLOS One 7:e46307).

The protein RGM-A, encoded by RGMA, is expressed in the central nervous system during embryonic development in a largely non-overlapping manner with RGM-B. In the adult, RGM-A is expressed in brain as well as many other tissues, and it has been implicated in cancer, immune regulation, and as a sarcoplasmic protein regulating differentiation and size of skeletal muscle cells (Tian et al., 2013, Mol Reprod Dev 80:700-717; Martins et al., 2014, Cells Tissues Organs 200:326-338). Studies of RGM-A in several cell types in vitro suggest that it increases BMP signaling by facilitating use of ActRIIA by endogenous BMP2 and BMP4 ligands that otherwise prefer signaling through BMPRII (Xia et al., 2007, J Biol Chem 282:18129-18140).

RGM-B, also known as DRAGON and encoded by RGMB. Like RGM-A, RGM-B is expressed in brain as well as many other tissues of the adult. RGM-B knockout mice die several weeks after birth for undetermined reasons (Xia et al., 2011, J Immunol 186:1369-1376). RGM-B binds BMP2 and BMP4 but not BMP7, activin A, or TGFβ isoforms, as determined by surface plasmon resonance, and interacts directly with type I receptors (ALK2, ALK3, and ALK6) and type II receptors (ActRIIA and ActRIIB), as determined by co-immunoprecipitation and blockade with dominant negative receptors (Samad et al., 2005, J Biol Chem 280:14122-14129). The ability of RGM-B to increase BMP signaling requires membrane association through its C-terminal GPI anchor.

The protein RGM-C, also known as hemojuvelin (HJV) and encoded by HFE2, is associated with juvenile hemochromatosis, a rare recessive disease characterized by early-onset systemic iron overload with severe clinical complications. Hemojuvelin is now known to be an essential factor in the regulation of hepcidin, a master regulator of iron homeostasis (Niederkofler et al., 2005, J Clin Invest 115:2180-2186). Hemojuvelin is expressed primarily in liver, consistent with the predominant site of hepcidin regulation, and also in heart and skeletal muscle, where the role of hemojuvelin is unclear. Multiple studies have demonstrated that hemojuvelin regulates hepcidin expression in the liver by altering BMP signaling. Unlike RGM-A and RGM-B, hemojuvelin binds with high affinity to BMP6, a key ligand regulating hepcidin expression (Andriopoulos et al., 2009, Nat Genet 41:482-487), in addition to binding BMP2 and BMP4. On the basis of siRNA knockdown experiments in cell lines and hepatic expression of superfamily proteins, it has been suggested that hemojuvelin promotes endogenous signaling of BMP2, BMP4, and BMP6 through ALK2 or ALK3 and ActRIIA (Xia et al., 2008, Blood 111:5195-5204).

In certain aspects, the present invention relates to MuSK polypeptides. Muscle-associated receptor tyrosine kinase (MuSK), also known as muscle-specific kinase, CMS9, or FADS, is encoded by MUSK. MuSK is a single-pass transmembrane protein originally identified as a receptor tyrosine kinase expressed prominently in embryonic skeletal muscle and at the mature neuromuscular junction (Valenzuela et al., 1995, Neuron 15:573-584). These investigators showed that MuSK expression is induced dramatically throughout the adult myofiber after denervation, blockade of electrical activity, or physical immobilization. Subsequent studies indicate that MuSK is activated by proteins structurally unrelated to the TGFβ superfamily in a complex temporal-spatial manner to promote and maintain clustering of acetylcholine receptors on the postsynaptic side of the neuromuscular junction and to induce differentiation of the presynaptic nerve terminal (Hubbard et al., 2013, Biochim Biophys Acta 1834:2166-2169). Surprisingly, recent studies have revealed that MuSK also serves as a BMP co-receptor which is capable of binding BMPs and type I receptors (ALK3, ALK6) and stimulating BMP signaling by a mechanism independent of MuSK tyrosine kinase activity (Yilmaz et al., 2016, Sci Signal 9:ra87).

The terms used in this specification generally have their ordinary meanings in the art, within the context of this disclosure and in the specific context where each term is used. Certain terms are discussed below or elsewhere in the specification to provide additional guidance to the practitioner in describing the compositions and methods of the disclosure and how to make and use them. The scope or meaning of any use of a term will be apparent from the specific context in which it is used.

The terms “heteromultimer complex”, “heteromer”, or “heteromultimer” is a complex comprising at least a first polypeptide and a second polypeptide, wherein the second polypeptide differs in amino acid sequence from the first polypeptide by at least one amino acid residue. The heteromer can comprise a “heterodimer” formed by the first and second polypeptide or can form higher order structures where polypeptides in addition to the first and second polypeptide are present. Exemplary structures for the heteromultimer include heterodimers, heterotrimers, heterotetramers and further oligomeric structures. Heterodimers are designated herein as X:Y or equivalently as X-Y, where X represents a first polypeptide and Y represents a second polypeptide. Higher-order heteromers and oligomeric structures are designated herein in a corresponding manner. In certain embodiments a heteromultimer is recombinant (e.g., one or more polypeptide components may be a recombinant protein), isolated and/or purified.

“Homologous,” in all its grammatical forms and spelling variations, refers to the relationship between two proteins that possess a “common evolutionary origin,” including proteins from superfamilies in the same species of organism, as well as homologous proteins from different species of organism. Such proteins (and their encoding nucleic acids) have sequence homology, as reflected by their sequence similarity, whether in terms of percent identity or by the presence of specific residues or motifs and conserved positions. However, in common usage and in the instant application, the term “homologous,” when modified with an adverb such as “highly,” may refer to sequence similarity and may or may not relate to a common evolutionary origin.

The term “sequence similarity,” in all its grammatical forms, refers to the degree of identity or correspondence between nucleic acid or amino acid sequences that may or may not share a common evolutionary origin.

“Percent (%) sequence identity” with respect to a reference polypeptide (or nucleotide) sequence is defined as the percentage of amino acid residues (or nucleic acids) in a candidate sequence that are identical to the amino acid residues (or nucleic acids) in the reference polypeptide (nucleotide) sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid (nucleic acid) sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

“Agonize”, in all its grammatical forms, refers to the process of activating a protein and/or gene (e.g., by activating or amplifying that protein's gene expression or by inducing an inactive protein to enter an active state) or increasing a protein's and/or gene's activity.

“Antagonize”, in all its grammatical forms, refers to the process of inhibiting a protein and/or gene (e.g., by inhibiting or decreasing that protein's gene expression or by inducing an active protein to enter an inactive state) or decreasing a protein's and/or gene's activity.

The terms “about” and “approximately” as used in connection with a numerical value throughout the specification and the claims denotes an interval of accuracy, familiar and acceptable to a person skilled in the art. In general, such interval of accuracy is ±10%, Alternatively, and particularly in biological systems, the terms “about” and “approximately” may mean values that are within an order of magnitude, preferably ≤5-fold and more preferably ≤2-fold of a given value.

Numeric ranges disclosed herein are inclusive of the numbers defining the ranges.

The terms “a” and “an” include plural referents unless the context in which the term is used clearly dictates otherwise. The terms “a” (or “an”), as well as the terms “one or more,” and “at least one” can be used interchangeably herein. Furthermore, “and/or” where used herein is to be taken as specific disclosure of each of the two or more specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

### 2. TGF-beta Superfamily Co-receptor, Type I Receptor, and Type II Receptor Polypeptides and Heteromultimers

In part, the disclosure provides recombinant TGF-beta superfamily heteromultimers (heteromultimers) comprising at least one TGF-beta superfamily co-receptor polypeptide (e.g., endoglin, betaglycan, Cripto-1, Cryptic, Cryptic family protein 1B, Crim1, Crim2, BAMBI, BMPER, RGM-A, RGM-B, hemojuvelin, and MuSK), including fragments and variants thereof. In some embodiments, the disclosure relates to a recombinant heteromultimer comprising a TGF-beta superfamily co-receptor polypeptide selected from the group consisting of: endoglin, betaglycan, Cripto-1, Cryptic, Cryptic family protein 1B, Crim1, Crim2, BAMBI, BMPER, RGM-A, RGM-B, hemojuvelin, and MuSK, including fragments and variants thereof, and a TGF-beta superfamily type I receptor polypeptide selected from the group consisting of: ALK1, ALK2, ALK3, ALK4, ALK5, ALK6, and ALK7, including fragments and variants thereof. In some embodiments, the disclosure relates to a recombinant heteromultimer comprising a TGF-beta superfamily co-receptor polypeptide selected from the group consisting of: endoglin, betaglycan, Cripto-1, Cryptic, Cryptic family protein 1B, Crim1, Crim2, BAMBI, BMPER, RGM-A, RGM-B, hemojuvelin, and MuSK, including fragments and variants thereof, and a TGF-beta superfamily type II receptor polypeptide selected from the group consisting of: ActRIIA, ActRIIB, TGFBRII, BMPRII, and MISRII, including fragments and variants thereof. In some embodiments, the disclosure relates to a recombinant heteromultimer comprising a first TGF-beta superfamily co-receptor polypeptide selected from the group consisting of: endoglin, betaglycan, Cripto-1, Cryptic, Cryptic family protein 1B, Crim1, Crim2, BAMBI, BMPER, RGM-A, RGM-B, hemojuvelin, and MuSK, including fragments and variants thereof, and a second TGF-beta superfamily co-receptor polypeptide selected from the group consisting of: endoglin, betaglycan, Cripto-1, Cryptic, Cryptic family protein 1B, Crim1, Crim2, BAMBI, BMPER, RGM-A, RGM-B, hemojuvelin, and MuSK, including fragments and variants thereof. Preferably, TGF-beta superfamily co-receptor, type I receptor, and type II receptor polypeptides as described herein comprise a ligand-binding domain of the receptor, for example, an extracellular domain of a TGF-beta superfamily co-receptor, type I receptor, or type II receptor. In other preferred embodiments, polypeptides and heteromultimers of the disclosure (e.g., co-receptor:type I receptor, co-receptor:type II receptor, and co-receptor:co-receptor heteromultimers) are soluble. In certain preferred embodiments, heteromultimers of the disclosure (e.g., co-receptor:type I receptor, co-receptor:type II receptor, and co-receptor:co-receptor heteromultimers) bind to one or more TGF-beta superfamily ligands (e.g., BMP2, BMP2/7, BMP3, BMP4, BMP4/7, BMP5, BMP6, BMP7, BMP8a, BMP8b, BMP9, BMP10, GDF3, GDF5, GDF6/BMP13, GDF7, GDF8, GDF9b/BMP15, GDF11/BMP11, GDF15/MIC1, TGF-β1, TGF-β2, TGF-β3, activin A, activin B, activin C, activin E, activin AB, activin AC, activin AE, activin BC, activin BE, nodal, glial cell-derived neurotrophic factor (GDNF), neurturin, artemin, persephin, Müllerian-inhibiting substance (MIS), and Lefty). In some embodiments, a heteromultimer (e.g., co-receptor:type I receptor, co-receptor:type II receptor, and co-receptor:co-receptor heteromultimers) may bind to one or more TGF-beta superfamily ligands with a K_{D} of at least 1×10⁻⁷ M (e.g., K_{D} of greater than or equal to 10⁻⁷, 10⁻⁸, 10⁻⁹, 10⁻¹⁰, 10⁻¹¹, or 10⁻¹²). In some embodiments, a heteromultimer of the disclosure (e.g., co-receptor:type I receptor, co-receptor:type II receptor, and co-receptor:co-receptor heteromultimers) has a different TGF-beta superfamily ligand binding and/or inhibition profile (specificity) compared to a corresponding homomultimer (e.g., endoglin:ALK1 heteromultimer vs. endoglin and ALK1 homomultimers). In some embodiments, a heteromultimer of the disclosure (e.g., co-receptor:type I receptor, co-receptor:type II receptor, and co-receptor:co-receptor heteromultimers) may inhibit one or more TGF-beta superfamily ligands (e.g., BMP2, BMP2/7, BMP3, BMP4, BMP4/7, BMP5, BMP6, BMP7, BMP8a, BMP8b, BMP9, BMP10, GDF3, GDF5, GDF6/BMP13, GDF7, GDF8, GDF9b/BMP15, GDF11/BMP11, GDF15/MIC1, TGF-β1, TGF-β2, TGF-β3, activin A, activin B, activin C, activin E, activin AB, activin AC, activin AE, activin BC, activin BE, nodal, glial cell-derived neurotrophic factor (GDNF), neurturin, artemin, persephin, Müllerian-inhibiting substance (MIS), and Lefty). In some embodiments, a heteromultimer of the disclosure (e.g., co-receptor:type I receptor, co-receptor:type II receptor, and co-receptor:co-receptor heteromultimers) may inhibit signaling of one or more TGF-beta superfamily ligands. For example, in some embodiments, a heteromultimer of the disclosure (e.g., co-receptor:type I receptor, co-receptor:type II receptor, and co-receptor:co-receptor heteromultimers) may inhibit signaling of one or more TGF-beta superfamily ligands in a cell-based assay (e.g., cell-based signaling assays as described herein). In some embodiments, heteromultimers of the disclosure are heterodimers.

As used herein, the term “ActRIIB” refers to a family of activin receptor type IIB (ActRIIB) proteins from any species and variants derived from such ActRIIB proteins by mutagenesis or other modification. Reference to ActRIIB herein is understood to be a reference to any one of the currently identified forms. Members of the ActRIIB family are generally transmembrane proteins, composed of a ligand-binding extracellular domain comprising a cysteine-rich region, a transmembrane domain, and a cytoplasmic domain with predicted serine/threonine kinase activity.

The term “ActRIIB polypeptide” includes polypeptides comprising any naturally occurring polypeptide of an ActRIIB family member as well as any variants thereof (including mutants, fragments, fusions, and peptidomimetic forms) that retain a useful activity. Examples of such variant ActRIIB polypeptides are provided throughout the present disclosure as well as in International Patent Application Publication Nos. WO 2006/012627, WO 2008/097541, and WO 2010/151426, which are incorporated herein by reference in their entirety.

The human ActRIIB precursor protein sequence is as follows:

| (SEQ ID NO: 1) | |
|---|---|
| 1 | MTAPWVALAL LWGSLCAGSG RGEAETRECI YYNANWELER TNQSGLERCE |
| 51 | GEQDKRLHCY ASWRNSSGTI ELVKKGCWLD DFNCYDRQEC VATEENPQVY |
| 101 | FCCCEGNFCN ERFTHLPEAG GPEVTYEPPP TAPTLLTVLA YSLLPIGGLS |
| 151 | LIVLLAFWMY RHRKPPYGHV DIHEDPGPPP PSPLVGLKPL QLLEIKARGR |
| 201 | FGCVWKAQLM NDFVAVKIFP LQDKQSWQSE REIFSTPGMK HENLLQFIAA |
| 251 | EKRGSNLEVE LWLITAFHDK GSLTDYLKGN IITWNELCHV AETMSRGLSY |
| 301 | LHEDVPWCRG EGHKPSIAHR DFKSKNVLLK SDLTAVLADF GLAVRFEPGK |
| 351 | PPGDTHGQVG TRRYMAPEVL EGAINFQRDA FLRIDMYAMG LVLWELVSRC |
| 401 | KAADGPVDEY MLPFEEEIGQ HPSLEELQEV VVHKKMRPTI KDHWLKHPGL |
| 451 | AQLCVTIEEC WDHDAEARLS AGCVEERVSL IRRSVNGTTS DCLVSLVTSV |
| 501 | TNVDLPPKES SI |

The signal peptide is indicated with a single underline; the extracellular domain is indicated in bold font; and the potential, endogenous N-linked glycosylation sites are indicated with a double underline.

A processed extracellular ActRIIB polypeptide sequence is as follows:

(SEQ ID NO: 2)

GRGEAETRECIYYNANWELERTNQSGLERCEGEQDKRLHCYASWRNSSGT
IELVKKGCWLDDFNCYDRQECVATEENPQVYFCCCEGNFCNERFTHLPEA
GGPEVTYEPPPTAP.

In some embodiments, the protein may be produced with an “SGR . . . ” sequence at the N-terminus. The C-terminal “tail” of the extracellular domain is indicated by a single underline. The sequence with the “tail” deleted (a 415 sequence) is as follows:

(SEQ ID NO: 3)

GRGEAETRECIYYNANWELERTNQSGLERCEGEQDKRLHCYASWRNSSGT
IELVKKGCWLDDFNCYDRQECVATEENPQVYFCCCEGNFCNERFTHLPE
A.

A form of ActRIIB with an alanine at position 64 of SEQ ID NO: 1 (A64) is also reported in the literature. See, e.g., Hilden et al. (1994) Blood, 83(8): 2163-2170. Applicants have ascertained that an ActRIIB-Fc fusion protein comprising an extracellular domain of ActRIIB with the A64 substitution has a relatively low affinity for activin and GDF11. By contrast, the same ActRIIB-Fc fusion protein with an arginine at position 64 (R64) has an affinity for activin and GDF11 in the low nanomolar to high picomolar range. Therefore, sequences with an R64 are used as the “wild-type” reference sequence for human ActRIIB in this disclosure.

The form of ActRIIB with an alanine at position 64 is as follows:

| (SEQ ID NO: 4) | |
|---|---|
| 1 | MTAPWVALAL LWGSLCAGSG RGEAETRECI YYNANWELER TNQSGLERCE |
| 51 | GEQDKRLHCY ASWANSSGTI ELVKKGCWLD DFNCYDRQEC VATEENPQVY |
| 101 | FCCCEGNFCN ERFTHLPEAG GPEVTYEPPP TAPTLLTVLA YSLLPIGGLS |
| 151 | LIVLLAFWMY RHRKPPYGHV DIHEDPGPPP PSPLVGLKPL QLLEIKARGR |
| 201 | FGCVWKAQLM NDFVAVKIFP LQDKQSWQSE REIFSTPGMK HENLLQFIAA |
| 251 | EKRGSNLEVE LWLITAFHDK GSLTDYLKGN IITWNELCHV AETMSRGLSY |
| 301 | LHEDVPWCRG EGHKPSIAHR DFKSKNVLLK SDLTAVLADF GLAVRFEPGK |
| 351 | PPGDTHGQVG TRRYMAPEVL EGAINFQRDA FLRIDMYAMG LVLWELVSRC |
| 401 | KAADGPVDEY MLPFEEEIGQ HPSLEELQEV VVHKKMRPTI KDHWLKHPGL |
| 451 | AQLCVTIEEC WDHDAEARLS AGCVEERVSL IRRSVNGTTS DCLVSLVTSV |
| 501 | TNVDLPPKES SI |

The signal peptide is indicated by single underline and the extracellular domain is indicated by bold font.

A processed extracellular ActRIIB polypeptide sequence of the alternative A64 form is as follows:

(SEQ ID NO: 5)

GRGEAETRECIYYNANWELERTNQSGLERCEGEQDKRLHCYASWANSSGT
IELVKKGCWLDDFNCYDRQECVATEENPQVYFCCCEGNFCNERFTHLPEA
GGPEVTYEPPPTAPT

In some embodiments, the protein may be produced with an “SGR . . . ” sequence at the N-terminus. The C-terminal “tail” of the extracellular domain is indicated by single underline. The sequence with the “tail” deleted (a 415 sequence) is as follows:

(SEQ ID NO: 6)

GRGEAETRECIYYNANWELERTNQSGLERCEGEQDKRLHCYASWANSSGT
IELVKKGCWLDDFNCYDRQECVATEENPQVYFCCCEGNFCNERFTHLPEA

A nucleic acid sequence encoding the human ActRIIB precursor protein is shown below (SEQ ID NO: 7), representing nucleotides 25-1560 of Genbank Reference Sequence NM_001106.3, which encode amino acids 1-513 of the ActRIIB precursor. The sequence as shown provides an arginine at position 64 and may be modified to provide an alanine instead. The signal sequence is underlined.

| (SEQ ID NO: 7) | |
|---|---|
| 1 | ATGACGGCGC CCTGGGTGGC CCTCGCCCTC CTCTGGGGAT CGCTGTGCGC |
| 51 | CGGCTCTGGG CGTGGGGAGG CTGAGACACG GGAGTGCATC TACTACAACG |
| 101 | CCAACTGGGA GCTGGAGCGC ACCAACCAGA GCGGCCTGGA GCGCTGCGAA |
| 151 | GGCGAGCAGG ACAAGCGGCT GCACTGCTAC GCCTCCTGGC GCAACAGCTC |
| 201 | TGGCACCATC GAGCTCGTGA AGAAGGGCTG CTGGCTAGAT GACTTCAACT |
| 251 | GCTACGATAG GCAGGAGTGT GTGGCCACTG AGGAGAACCC CCAGGTGTAC |
| 301 | TTCTGCTGCT GTGAAGGCAA CTTCTGCAAC GAACGCTTCA CTCATTTGCC |
| 351 | AGAGGCTGGG GGCCCGGAAG TCACGTACGA GCCACCCCCG ACAGCCCCCA |
| 401 | CCCTGCTCAC GGTGCTGGCC TACTCACTGC TGCCCATCGG GGGCCTTTCC |
| 451 | CTCATCGTCC TGCTGGCCTT TTGGATGTAC CGGCATCGCA AGCCCCCCTA |
| 501 | CGGTCATGTG GACATCCATG AGGACCCTGG GCCTCCACCA CCATCCCCTC |
| 551 | TGGTGGGCCT GAAGCCACTG CAGCTGCTGG AGATCAAGGC TCGGGGGCGC |
| 601 | TTTGGCTGTG TCTGGAAGGC CCAGCTCATG AATGACTTTG TAGCTGTCAA |
| 651 | GATCTTCCCA CTCCAGGACA AGCAGTCGTG GCAGAGTGAA CGGGAGATCT |
| 701 | TCAGCACACC TGGCATGAAG CACGAGAACC TGCTACAGTT CATTGCTGCC |
| 751 | GAGAAGCGAG GCTCCAACCT CGAAGTAGAG CTGTGGCTCA TCACGGCCTT |
| 801 | CCATGACAAG GGCTCCCTCA CGGATTACCT CAAGGGGAAC ATCATCACAT |
| 851 | GGAACGAACT GTGTCATGTA GCAGAGACGA TGTCACGAGG CCTCTCATAC |
| 901 | CTGCATGAGG ATGTGCCCTG GTGCCGTGGC GAGGGCCACA AGCCGTCTAT |
| 951 | TGCCCACAGG GACTTTAAAA GTAAGAATGT ATTGCTGAAG AGCGACCTCA |
| 1001 | CAGCCGTGCT GGCTGACTTT GGCTTGGCTG TTCGATTTGA GCCAGGGAAA |
| 1051 | CCTCCAGGGG ACACCCACGG ACAGGTAGGC ACGAGACGGT ACATGGCTCC |
| 1101 | TGAGGTGCTC GAGGGAGCCA TCAACTTCCA GAGAGATGCC TTCCTGCGCA |
| 1151 | TTGACATGTA TGCCATGGGG TTGGTGCTGT GGGAGCTTGT GTCTCGCTGC |
| 1201 | AAGGCTGCAG ACGGACCCGT GGATGAGTAC ATGCTGCCCT TTGAGGAAGA |
| 1251 | GATTGGCCAG CACCCTTCGT TGGAGGAGCT GCAGGAGGTG GTGGTGCACA |
| 1301 | AGAAGATGAG GCCCACCATT AAAGATCACT GGTTGAAACA CCCGGGCCTG |
| 1351 | GCCCAGCTTT GTGTGACCAT CGAGGAGTGC TGGGACCATG ATGCAGAGGC |
| 1401 | TCGCTTGTCC GCGGGCTGTG TGGAGGAGCG GGTGTCCCTG ATTCGGAGGT |
| 1451 | CGGTCAACGG CACTACCTCG GACTGTCTCG TTTCCCTGGT GACCTCTGTC |
| 1501 | ACCAATGTGG ACCTGCCCCC TAAAGAGTCA AGCATC |

A nucleic acid sequence encoding processed extracellular human ActRIIB polypeptide is as follows (SEQ ID NO: 8). The sequence as shown provides an arginine at position 64, and may be modified to provide an alanine instead.

| (SEQ ID NO: 8) | |
|---|---|
| 1 | GGGCGTGGGG AGGCTGAGAC ACGGGAGTGC ATCTACTACA ACGCCAACTG |
| 51 | GGAGCTGGAG CGCACCAACC AGAGCGGCCT GGAGCGCTGC GAAGGCGAGC |
| 101 | AGGACAAGCG GCTGCACTGC TACGCCTCCT GGCGCAACAG CTCTGGCACC |
| 151 | ATCGAGCTCG TGAAGAAGGG CTGCTGGCTA GATGACTTCA ACTGCTACGA |
| 201 | TAGGCAGGAG TGTGTGGCCA CTGAGGAGAA CCCCCAGGTG TACTTCTGCT |
| 251 | GCTGTGAAGG CAACTTCTGC AACGAACGCT TCACTCATTT GCCAGAGGCT |
| 301 | GGGGGCCCGG AAGTCACGTA CGAGCCACCC CCGACAGCCC CCACC |

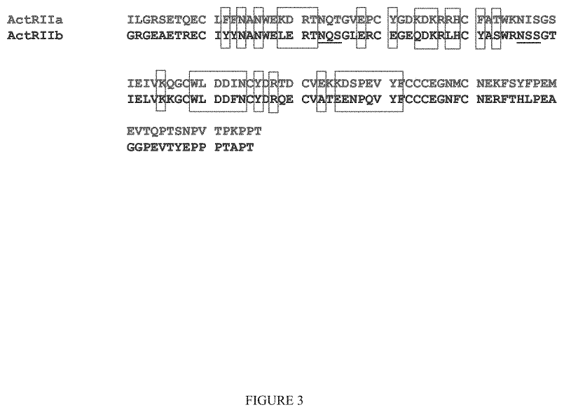

An alignment of the amino acid sequences of human ActRIIB extracellular domain and human ActRIIA extracellular domain are illustrated in FIG. 3. This alignment indicates amino acid residues within both receptors that are believed to directly contact ActRII ligands. For example, the composite ActRII structures indicated that the ActRIIB-ligand binding pocket is defined, in part, by residues Y31, N33, N35, L38 through T41, E47, E50, Q53 through K55, L57, H58, Y60, S62, K74, W78 through N83, Y85, R87, A92, and E94 through F101. At these positions, it is expected that conservative mutations will be tolerated.

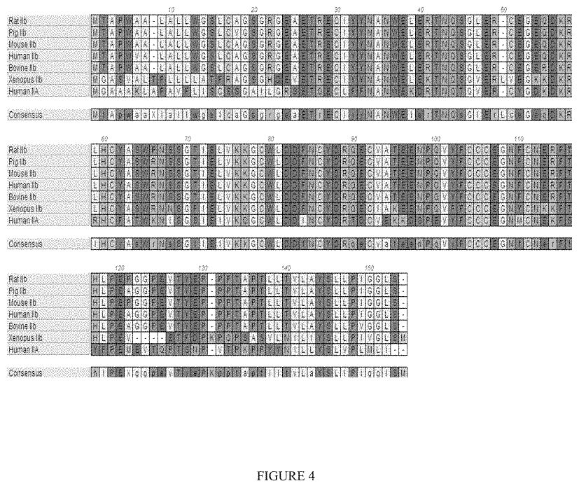

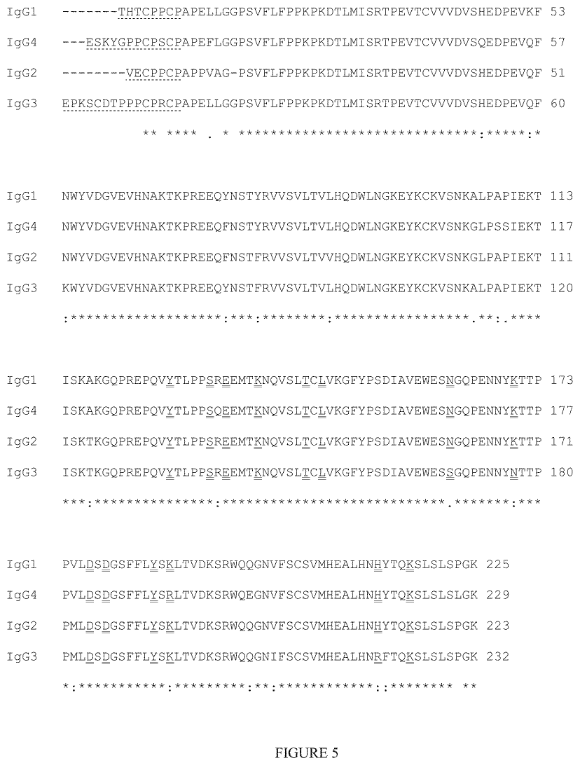

In addition, ActRIIB is well-conserved among vertebrates, with large stretches of the extracellular domain completely conserved. For example, FIG. 4 depicts a multi-sequence alignment of a human ActRIIB extracellular domain compared to various ActRIIB orthologs. Many of the ligands that bind to ActRIIB are also highly conserved. Accordingly, from these alignments, it is possible to predict key amino acid positions within the ligand-binding domain that are important for normal ActRIIB-ligand binding activities as well as to predict amino acid positions that are likely to be tolerant of substitution without significantly altering normal ActRIIB-ligand binding activities. Therefore, an active, human ActRIIB variant polypeptide useful in accordance with the presently disclosed methods may include one or more amino acids at corresponding positions from the sequence of another vertebrate ActRIIB, or may include a residue that is similar to that in the human or other vertebrate sequences. Without meaning to be limiting, the following examples illustrate this approach to defining an active ActRIIB variant. L46 in the human extracellular domain (SEQ ID NO: 2) is a valine in Xenopus ActRIIB and so this position may be altered, and optionally may be altered to another hydrophobic residue, such as V, I or F, or a non-polar residue such as A. E52 in the human extracellular domain is a K in Xenopus, indicating that this site may be tolerant of a wide variety of changes, including polar residues, such as E, D, K, R, H, S, T, P, G, Y and probably A. T93 in the human extracellular domain is a K in Xenopus, indicating that a wide structural variation is tolerated at this position, with polar residues favored, such as S, K, R, E, D, H, G, P, G and Y. F108 in the human extracellular domain is a Y in Xenopus, and therefore Y or other hydrophobic group, such as I, V or L should be tolerated. E111 in the human extracellular domain is K in Xenopus, indicating that charged residues will be tolerated at this position, including D, R, K and H, as well as Q and N. R112 in the human extracellular domain is K in Xenopus, indicating that basic residues are tolerated at this position, including R and H. A at position 119 in the human extracellular domain is relatively poorly conserved, and appears as P in rodents and V in Xenopus, thus essentially any amino acid should be tolerated at this position.

Moreover, ActRII proteins have been characterized in the art in terms of structural and functional characteristics, particularly with respect to ligand binding [Attisano et al. (1992) Cell 68(1):97-108; Greenwald et al. (1999) Nature Structural Biology 6(1): 18-22; Allendorph et al. (2006) PNAS 103(20: 7643-7648; Thompson et al. (2003) The EMBO Journal 22(7): 1555-1566; as well as U.S. Pat. Nos. 7,709,605, 7,612,041, and 7,842,663]. In addition to the teachings herein, these references provide amply guidance for how to generate ActRIIB variants that retain one or more normal activities (e.g., ligand-binding activity).

For example, a defining structural motif known as a three-finger toxin fold is important for ligand binding by type I and type II receptors and is formed by conserved cysteine residues located at varying positions within the extracellular domain of each monomeric receptor [Greenwald et al. (1999) Nat Struct Biol 6:18-22; and Hinck (2012) FEBS Lett 586:1860-1870]. Accordingly, the core ligand-binding domains of human ActRIIB, as demarcated by the outermost of these conserved cysteines, corresponds to positions 29-109 of SEQ ID NO: 1 (ActRIIB precursor). Thus, the structurally less-ordered amino acids flanking these cysteine-demarcated core sequences can be truncated by about 1, 2, 3, 4, 5, 6, 7, 8, 9, 10, 11, 12, 13, 14, 15, 16, 17, 18, 19, 20, 21, 22, 23, 24, 25, 26, 27, or 28 residues at the N-terminus and/or by about 1, 2, 3, 4, 5, 6, 7, 8, 9, 10, 11, 12, 13, 14, 15, 16, 17, 18, 19, 20, 21, 22, 23, 24, or 25 residues a the C-terminus without necessarily altering ligand binding. Exemplary ActRIIB extracellular domains for N-terminal and/or C-terminal truncation include SEQ ID NOs: 2, 3, 5, and 6.

Attisano et al. showed that a deletion of the proline knot at the C-terminus of the extracellular domain of ActRIIB reduced the affinity of the receptor for activin. An ActRIIB-Fc fusion protein containing amino acids 20-119 of present SEQ ID NO: 1, “ActRIIB(20-119)-Fc”, has reduced binding to GDF11 and activin relative to an ActRIIB(20-134)-Fc, which includes the proline knot region and the complete juxtamembrane domain (see, e.g., U.S. Pat. No. 7,842,663). However, an ActRIIB(20-129)-Fc protein retains similar, but somewhat reduced activity, relative to the wild-type, even though the proline knot region is disrupted.

Thus, ActRIIB extracellular domains that stop at amino acid 134, 133, 132, 131, 130 and 129 (with respect to SEQ ID NO: 1) are all expected to be active, but constructs stopping at 134 or 133 may be most active. Similarly, mutations at any of residues 129-134 (with respect to SEQ ID NO: 1) are not expected to alter ligand-binding affinity by large margins. In support of this, it is known in the art that mutations of P129 and P130 (with respect to SEQ ID NO: 1) do not substantially decrease ligand binding. Therefore, an ActRIIB polypeptide of the present disclosure may end as early as amino acid 109 (the final cysteine), however, forms ending at or between 109 and 119 (e.g., 109, 110, 111, 112, 113, 114, 115, 116, 117, 118, or 119) are expected to have reduced ligand binding. Amino acid 119 (with respect to present SEQ ID NO: 1) is poorly conserved and so is readily altered or truncated. ActRIIB polypeptides ending at 128 (with respect to SEQ ID NO: 1) or later should retain ligand-binding activity. ActRIIB polypeptides ending at or between 119 and 127 (e.g., 119, 120, 121, 122, 123, 124, 125, 126, or 127), with respect to SEQ ID NO: 1, will have an intermediate binding ability. Any of these forms may be desirable to use, depending on the clinical or experimental setting.

At the N-terminus of ActRIIB, it is expected that a protein beginning at amino acid 29 or before (with respect to SEQ ID NO: 1) will retain ligand-binding activity. Amino acid 29 represents the initial cysteine. An alanine-to-asparagine mutation at position 24 (with respect to SEQ ID NO: 1) introduces an N-linked glycosylation sequence without substantially affecting ligand binding [U.S. Pat. No. 7,842,663]. This confirms that mutations in the region between the signal cleavage peptide and the cysteine cross-linked region, corresponding to amino acids 20-29, are well tolerated. In particular, ActRIIB polypeptides beginning at position 20, 21, 22, 23, and 24 (with respect to SEQ ID NO: 1) should retain general ligand-biding activity, and ActRIIB polypeptides beginning at positions 25, 26, 27, 28, and 29 (with respect to SEQ ID NO: 1) are also expected to retain ligand-biding activity. It has been demonstrated, e.g., U.S. Pat. No. 7,842,663, that, surprisingly, an ActRIIB construct beginning at 22, 23, 24, or 25 will have the most activity.

Taken together, a general formula for an active portion (e.g., ligand-binding portion) of ActRIIB comprises amino acids 29-109 of SEQ ID NO: 1. Therefore ActRIIB polypeptides may, for example, comprise, consist essentially of, or consist of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to a portion of ActRIIB beginning at a residue corresponding to any one of amino acids 20-29 (e.g., beginning at any one of amino acids 20, 21, 22, 23, 24, 25, 26, 27, 28, or 29) of SEQ ID NO: 1 and ending at a position corresponding to any one amino acids 109-134 (e.g., ending at any one of amino acids 109, 110, 111, 112, 113, 114, 115, 116, 117, 118, 119, 120, 121, 122, 123, 124, 125, 126, 127, 128, 129, 130, 131, 132, 133, or 134) of SEQ ID NO: 1. Other examples include polypeptides that begin at a position from 20-29 (e.g., any one of positions 20, 21, 22, 23, 24, 25, 26, 27, 28, or 29) or 21-29 (e.g., any one of positions 21, 22, 23, 24, 25, 26, 27, 28, or 29) of SEQ ID NO: 1 and end at a position from 119-134 (e.g., any one of positions 119, 120, 121, 122, 123, 124, 125, 126, 127, 128, 129, 130, 131, 132, 133, or 134), 119-133 (e.g., any one of positions 119, 120, 121, 122, 123, 124, 125, 126, 127, 128, 129, 130, 131, 132, or 133), 129-134 (e.g., any one of positions 129, 130, 131, 132, 133, or 134), or 129-133 (e.g., any one of positions 129, 130, 131, 132, or 133) of SEQ ID NO: 1. Other examples include constructs that begin at a position from 20-24 (e.g., any one of positions 20, 21, 22, 23, or 24), 21-24 (e.g., any one of positions 21, 22, 23, or 24), or 22-25 (e.g., any one of positions 22, 22, 23, or 25) of SEQ ID NO: 1 and end at a position from 109-134 (e.g., any one of positions 109, 110, 111, 112, 113, 114, 115, 116, 117, 118, 119, 120, 121, 122, 123, 124, 125, 126, 127, 128, 129, 130, 131, 132, 133, or 134), 119-134 (e.g., any one of positions 119, 120, 121, 122, 123, 124, 125, 126, 127, 128, 129, 130, 131, 132, 133, or 134) or 129-134 (e.g., any one of positions 129, 130, 131, 132, 133, or 134) of SEQ ID NO: 1. Variants within these ranges are also contemplated, particularly those having at least 70%, 75%, 80%, 85%, 86%, 87%, 88%, 89%, 90%, 91%, 92%, 93%, 94%, 95%, 96%, 97%, 98%, 99%, or 100% identity to the corresponding portion of SEQ ID NO: 1.

The variations described herein may be combined in various ways. In some embodiments, ActRIIB variants comprise no more than 1, 2, 5, 6, 7, 8, 9, 10 or 15 conservative amino acid changes in the ligand-binding pocket, and zero, one, or more non-conservative alterations at positions 40, 53, 55, 74, 79 and/or 82 in the ligand-binding pocket. Sites outside the binding pocket, at which variability may be particularly well tolerated, include the amino and carboxy termini of the extracellular domain (as noted above), and positions 42-46 and 65-73 (with respect to SEQ ID NO: 1). An asparagine-to-alanine alteration at position 65 (N65A) actually improves ligand binding in the A64 background, and is thus expected to have no detrimental effect on ligand binding in the R64 background [U.S. Pat. No. 7,842,663]. This change probably eliminates glycosylation at N65 in the A64 background, thus demonstrating that a significant change in this region is likely to be tolerated. While an R64A change is poorly tolerated, R64K is well-tolerated, and thus another basic residue, such as H may be tolerated at position 64 [U.S. Pat. No. 7,842,663]. Additionally, the results of the mutagenesis program described in the art indicate that there are amino acid positions in ActRIIB that are often beneficial to conserve. With respect to SEQ ID NO: 1, these include position 80 (acidic or hydrophobic amino acid), position 78 (hydrophobic, and particularly tryptophan), position 37 (acidic, and particularly aspartic or glutamic acid), position 56 (basic amino acid), position 60 (hydrophobic amino acid, particularly phenylalanine or tyrosine). Thus, the disclosure provides a framework of amino acids that may be conserved in ActRIIB polypeptides. Other positions that may be desirable to conserve are as follows: position 52 (acidic amino acid), position 55 (basic amino acid), position 81 (acidic), 98 (polar or charged, particularly E, D, R or K), all with respect to SEQ ID NO: 1.

In certain embodiments, the disclosure relates to heteromultimers that comprise at least one ActRIIB polypeptide, which includes fragments, functional variants, and modified forms thereof. Preferably, ActRIIB polypeptides for use in accordance with the disclosure are soluble (e.g., an extracellular domain of ActRIIB). In other preferred embodiments, ActRIIB polypeptides for use in accordance with the disclosure bind to one or more TGF-beta superfamily ligands. Therefore, in some embodiments, ActRIIB polypeptides for use in accordance with the disclosure inhibit (antagonize) activity (e.g., Smad signaling) of one or more TGF-beta superfamily ligands. In some embodiments, heteromultimers of the disclosure comprise at least one ActRIIB polypeptide that comprises an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to a portion of ActRIIB beginning at a residue corresponding to amino acids 20-29 (e.g., beginning at any one of amino acids 20, 21, 22, 23, 24, 25, 26, 27, 28, or 29) of SEQ ID NO: 1 and ending at a position corresponding to amino acids 109-134 (e.g., ending at any one of amino acids 109, 110, 111, 112, 113, 114, 115, 116, 117, 118, 119, 120, 121, 122, 123, 124, 125, 126, 127, 128, 129, 130, 131, 132, 133, or 134) of SEQ ID NO: 1. In certain preferred embodiments, heteromultimers of the disclosure comprise at least one ActRIIB polypeptide that comprises amino acid sequence that is at least 70%, 75%, 80%, 85%, 86%, 87%, 88%, 89%, 90%, 91%, 92%, 93%, 94%, 95%, 96%, 97%, 98%, 99%, or 100% identical amino acids 29-109 of SEQ ID NO: 1 In other preferred embodiments, heteromultimers of the disclosure comprise at least one ActRIIB polypeptide that comprises an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical amino acids 25-131 of SEQ ID NO: 1 In some embodiments, heteromultimers of the disclosure comprise at least one ActRIIB polypeptide that is at least 70%, 75%, 80%, 85%, 86%, 87%, 88%, 89%, 90%, 91%, 92%, 93%, 94%, 95%, 97%, 98%, 99%, or 100% identical to the amino acid sequence of any one of SEQ ID NOs: 1, 2, 3, 4, 5, and 6. In certain embodiments, heteromultimers of the disclosure comprise at least one ActRIIB polypeptide wherein the amino acid position corresponding to L79 of SEQ ID NO: 1 is not an acidic amino acid (i.e., is not a naturally occurring D or E amino acid residue or artificial acidic amino acid).

In certain embodiments, the present disclosure relates to a protein complex comprising an ActRIIA polypeptide. As used herein, the term “ActRIIA” refers to a family of activin receptor type IIA (ActRIIA) proteins from any species and variants derived from such ActRIIA proteins by mutagenesis or other modification. Reference to ActRIIA herein is understood to be a reference to any one of the currently identified forms. Members of the ActRIIA family are generally transmembrane proteins, composed of a ligand-binding extracellular domain comprising a cysteine-rich region, a transmembrane domain, and a cytoplasmic domain with predicted serine/threonine kinase activity.

The term “ActRIIA polypeptide” includes polypeptides comprising any naturally occurring polypeptide of an ActRIIA family member as well as any variants thereof (including mutants, fragments, fusions, and peptidomimetic forms) that retain a useful activity. Examples of such variant ActRIIA polypeptides are provided throughout the present disclosure as well as in International Patent Application Publication No. WO 2006/012627, which is incorporated herein by reference in its entirety.

The human ActRIIA precursor protein sequence is as follows:

| (SEQ ID NO: 9) | |
|---|---|
| 1 | MGAAAKLAFA VFLISCSSGA ILGRSETQEC LFFNANWEKD RTNQTGVEPC |
| 51 | YGDKDKRRHC FATWKNISGS IEIVKQGCWL DDINCYDRTD CVEKKDSPEV |
| 101 | YFCCCEGNMC NEKFSYFPEM EVTQPTSNPV TPKPPYYNIL LYSLVPLMLI |
| 151 | AGIVICAFWV YRHHKMAYPP VLVPTQDPGP PPPSPLLGLK PLQLLEVKAR |
| 201 | GRFGCVWKAQ LLNEYVAVKI FPIQDKQSWQ NEYEVYSLPG MKHENILQFI |
| 251 | GAEKRGTSVD VDLWLITAFH EKGSLSDFLK ANVVSWNELC HIAETMARGL |
| 301 | AYLHEDIPGL KDGHKPAISH RDIKSKNVLL KNNLTACIAD FGLALKFEAG |
| 351 | KSAGDTHGQV GTRRYMAPEV LEGAINFQRD AFLRIDMYAM GLVLWELASR |
| 401 | CTAADGPVDE YMLPFEEEIG QHPSLEDMQE VVVHKKKRPV LRDYWQKHAG |
| 451 | MAMLCETIEE CWDHDAEARL SAGCVGERIT QMQRLTNIIT TEDIVTVVTM |
| 501 | VTNVDFPPKE SSL |

The signal peptide is indicated by a single underline; the extracellular domain is indicated in bold font; and the potential, endogenous N-linked glycosylation sites are indicated by a double underline.

A processed extracellular human ActRIIA polypeptide sequence is as follows:

(SEQ ID NO: 10)

ILGRSETQECLFFNANWEKDRTNQTGVEPCYGDKDKRRHCFATWKNISGS
IEIVKQGCWLDDINCYDRTDCVEKKDSPEVYFCCCEGNMCNEKFSYFPEM
EVTQPTSNPVTPKPP

The C-terminal “tail” of the extracellular domain is indicated by a single underline. The sequence with the “tail” deleted (a 415 sequence) is as follows:

(SEQ ID NO: 11)

ILGRSETQECLFFNANWEKDRTNQTGVEPCYGDKDKRRHCFATWKNISGS
IEIVKQGCWLDDINCYDRTDCVEKKDSPEVYFCCCEGNMCNEKFSYFPEM

A nucleic acid sequence encoding the human ActRIIA precursor protein is shown below (SEQ ID NO: 12), corresponding to nucleotides 159-1700 of Genbank Reference Sequence NM_001616.4. The signal sequence is underlined.

| (SEQ ID NO: 12) | |
|---|---|
| 1 | ATGGGAGCTG CTGCAAAGTT GGCGTTTGCC GTCTTTCTTA TCTCCTGTTC |
| 51 | TTCAGGTGCT ATACTTGGTA GATCAGAAAC TCAGGAGTGT CTTTTCTTTA |
| 101 | ATGCTAATTG GGAAAAAGAC AGAACCAATC AAACTGGTGT TGAACCGTGT |
| 151 | TATGGTGACA AAGATAAACG GCGGCATTGT TTTGCTACCT GGAAGAATAT |
| 201 | TTCTGGTTCC ATTGAAATAG TGAAACAAGG TTGTTGGCTG GATGATATCA |
| 251 | ACTGCTATGA CAGGACTGAT TGTGTAGAAA AAAAAGACAG CCCTGAAGTA |
| 301 | TATTTTTGTT GCTGTGAGGG CAATATGTGT AATGAAAAGT TTTCTTATTT |
| 351 | TCCGGAGATG GAAGTCACAC AGCCCACTTC AAATCCAGTT ACACCTAAGC |
| 401 | CACCCTATTA CAACATCCTG CTCTATTCCT TGGTGCCACT TATGTTAATT |
| 451 | GCGGGGATTG TCATTTGTGC ATTTTGGGTG TACAGGCATC ACAAGATGGC |
| 501 | CTACCCTCCT GTACTTGTTC CAACTCAAGA CCCAGGACCA CCCCCACCTT |
| 551 | CTCCATTACT AGGTTTGAAA CCACTGCAGT TATTAGAAGT GAAAGCAAGG |
| 601 | GGAAGATTTG GTTGTGTCTG GAAAGCCCAG TTGCTTAACG AATATGTGGC |
| 651 | TGTCAAAATA TTTCCAATAC AGGACAAACA GTCATGGCAA AATGAATACG |
| 701 | AAGTCTACAG TTTGCCTGGA ATGAAGCATG AGAACATATT ACAGTTCATT |
| 751 | GGTGCAGAAA AACGAGGCAC CAGTGTTGAT GTGGATCTTT GGCTGATCAC |
| 801 | AGCATTTCAT GAAAAGGGTT CACTATCAGA CTTTCTTAAG GCTAATGTGG |
| 851 | TCTCTTGGAA TGAACTGTGT CATATTGCAG AAACCATGGC TAGAGGATTG |
| 901 | GCATATTTAC ATGAGGATAT ACCTGGCCTA AAAGATGGCC ACAAACCTGC |
| 951 | CATATCTCAC AGGGACATCA AAAGTAAAAA TGTGCTGTTG AAAAACAACC |
| 1001 | TGACAGCTTG CATTGCTGAC TTTGGGTTGG CCTTAAAATT TGAGGCTGGC |
| 1051 | AAGTCTGCAG GCGATACCCA TGGACAGGTT GGTACCCGGA GGTACATGGC |
| 1101 | TCCAGAGGTA TTAGAGGGTG CTATAAACTT CCAAAGGGAT GCATTTTTGA |
| 1151 | GGATAGATAT GTATGCCATG GGATTAGTCC TATGGGAACT GGCTTCTCGC |
| 1201 | TGTACTGCTG CAGATGGACC TGTAGATGAA TACATGTTGC CATTTGAGGA |
| 1251 | GGAAATTGGC CAGCATCCAT CTCTTGAAGA CATGCAGGAA GTTGTTGTGC |
| 1301 | ATAAAAAAAA GAGGCCTGTT TTAAGAGATT ATTGGCAGAA ACATGCTGGA |
| 1351 | ATGGCAATGC TCTGTGAAAC CATTGAAGAA TGTTGGGATC ACGACGCAGA |
| 1401 | AGCCAGGTTA TCAGCTGGAT GTGTAGGTGA AAGAATTACC CAGATGCAGA |
| 1451 | GACTAACAAA TATTATTACC ACAGAGGACA TTGTAACAGT GGTCACAATG |
| 1501 | GTGACAAATG TTGACTTTCC TCCCAAAGAA TCTAGTCTA |

A nucleic acid sequence encoding a processed extracellular ActRIIA polypeptide is as follows:

| (SEQ ID NO: 13) | |
|---|---|
| 1 | ATACTTGGTA GATCAGAAAC TCAGGAGTGT CTTTTCTTTA ATGCTAATTG |
| 51 | GGAAAAAGAC AGAACCAATC AAACTGGTGT TGAACCGTGT TATGGTGACA |
| 101 | AAGATAAACG GCGGCATTGT TTTGCTACCT GGAAGAATAT TTCTGGTTCC |
| 151 | ATTGAAATAG TGAAACAAGG TTGTTGGCTG GATGATATCA ACTGCTATGA |
| 201 | CAGGACTGAT TGTGTAGAAA AAAAAGACAG CCCTGAAGTA TATTTTTGTT |
| 251 | GCTGTGAGGG CAATATGTGT AATGAAAAGT TTTCTTATTT TCCGGAGATG |
| 301 | GAAGTCACAC AGCCCACTTC AAATCCAGTT ACACCTAAGC CACCC |

A general formula for an active (e.g., ligand binding) ActRIIA polypeptide is one that comprises a polypeptide that starts at amino acid 30 and ends at amino acid 110 of SEQ ID NO: 9. Accordingly, ActRIIA polypeptides of the present disclosure may comprise a polypeptide that is at least 70%, 75%, 80%, 85%, 86%, 87%, 88%, 89%, 90%, 91%, 92%, 93%, 94%, 95%, 97%, 98%, 99%, or 100% identical to amino acids 30-110 of SEQ ID NO: 9. Optionally, ActRIIA polypeptides of the present disclosure comprise a polypeptide that is at least 70%, 75%, 80%, 85%, 86%, 87%, 88%, 89%, 90%, 91%, 92%, 93%, 94%, 95%, 97%, 98%, 99%, or 100% identical to amino acids 12-82 of SEQ ID NO: 9 optionally beginning at a position ranging from 1-5 (e.g., 1, 2, 3, 4, or 5) or 3-5 (e.g., 3, 4, or 5) and ending at a position ranging from 110-116 (e.g., 110, 111, 112, 113, 114, 115, or 116) or 110-115 (e.g., 110, 111, 112, 113, 114, or 115), respectively, and comprising no more than 1, 2, 5, 10 or 15 conservative amino acid changes in the ligand binding pocket, and zero, one or more non-conservative alterations at positions 40, 53, 55, 74, 79 and/or 82 in the ligand-binding pocket with respect to SEQ ID NO: 9.

In certain embodiments, the disclosure relates to heteromultimers that comprise at least one ActRIIA polypeptide, which includes fragments, functional variants, and modified forms thereof. Preferably, ActRIIA polypeptides for use in accordance with the disclosure (e.g., heteromultimer complexes comprising an ActRIIA polypeptide and uses thereof) are soluble (e.g., an extracellular domain of ActRIIA). In other preferred embodiments, ActRIIA polypeptides for use in accordance with the disclosure bind to and/or inhibit (antagonize) activity (e.g., Smad signaling) of one or more TGF-beta superfamily ligands. In some embodiments, heteromultimers of the disclosure comprise at least one ActRIIA polypeptide that is at least 70%, 75%, 80%, 85%, 86%, 87%, 88%, 89%, 90%, 91%, 92%, 93%, 94%, 95%, 97%, 98%, 99%, or 100% identical to the amino acid sequence of any one of SEQ ID NOs: 9, 10, and 11. In some embodiments, heteromultimers of the disclosure comprise at least one ActRIIA polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 21-30 (e.g., amino acid residues 21, 22, 23, 24, 25, 26, 27, 28, 29, or 30) of SEQ ID NO: 9, and ends at any one of amino acids 110-135 (e.g., amino acid residues 110, 111, 112, 113, 114, 115, 116, 117, 118, 119, 120, 121, 122, 123, 124, 125, 126, 127, 128, 129, 130, 131, 132, 133, 134, or 135) of SEQ ID NO: 9. In some embodiments, heteromultimers of the disclosure comprise at least one endoglin polypeptide that is at least 70%, 75%, 80%, 85%, 90%, 91%, 92%, 93%, 94%, 95%, 96%, 97%, 98%, 99%, or 100% identical to amino acids of 21-110 of SEQ ID NO: 9. In some embodiments, heteromultimers of the disclosure comprise at least one endoglin polypeptide that is at least 70%, 75%, 80%, 85%, 90%, 91%, 92%, 93%, 94%, 95%, 96%, 97%, 98%, 99%, or 100% identical to amino acids of 21-135 of SEQ ID NO: 9. In some embodiments, heteromultimers of the disclosure comprise at least one endoglin polypeptide that is at least 70%, 75%, 80%, 85%, 90%, 91%, 92%, 93%, 94%, 95%, 96%, 97%, 98%, 99%, or 100% identical to amino acids of 30-110 of SEQ ID NO: 9. In some embodiments, heteromultimers of the disclosure comprise at least one endoglin polypeptide that is at least 70%, 75%, 80%, 85%, 90%, 91%, 92%, 93%, 94%, 95%, 96%, 97%, 98%, 99%, or 100% identical to amino acids of 30-135 of SEQ ID NO: 9.

In certain aspects, the present disclosure relates to heteromultimers that comprise a TGFBRII polypeptide. As used herein, the term “TGFBRII” refers to a family of transforming growth factor-beta receptor II (TGFBRII) proteins from any species and variants derived from such proteins by mutagenesis or other modification. Reference to TGFBRII herein is understood to be a reference to any one of the currently identified forms. Members of the TGFBRII family are generally transmembrane proteins, composed of a ligand-binding extracellular domain with a cysteine-rich region, a transmembrane domain, and a cytoplasmic domain with predicted serine/threonine kinase activity.

The term “TGFBRII polypeptide” includes polypeptides comprising any naturally occurring polypeptide of a TGFBRII family member as well as any variants thereof (including mutants, fragments, fusions, and peptidomimetic forms) that retain a useful activity.

A human TGFBRII precursor protein sequence (NCBI Ref Seq NP_003233.4) is as follows:

| (SEQ ID NO: 42) | |
|---|---|
| 1 | MGRGLLRGLW PLHIVLWTRI ASTIPPHVQK SVNNDMIVTD NNGAVKFPQL |
| 51 | CKFCDVRFST CDNQKSCMSN CSITSICEKP QEVCVAVWRK NDENITLETV |
| 101 | CHDPKLPYHD FILEDAASPK CIMKEKKKPG ETFFMCSCSS DECNDNIIFS |
| 151 | EEYNTSNPDL LLVIFQVTGI SLLPPLGVAI SVIIIFYCYR VNRQQKLSST |
| 201 | WETGKTRKLM EFSEHCAIIL EDDRSDISST CANNINHNTE LLPIELDTLV |
| 251 | GKGRFAEVYK AKLKQNTSEQ FETVAVKIFP YEEYASWKTE KDIFSDINLK |
| 301 | HENILQFLTA EERKTELGKQ YWLITAFHAK GNLQEYLTRH VISWEDLRKL |
| 351 | GSSLARGIAH LHSDHTPCGR PKMPIVHRDL KSSNILVKND LTCCLCDFGL |
| 401 | SLRLDPTLSV DDLANSGQVG TARYMAPEVL ESRMNLENVE SFKQTDVYSM |
| 451 | ALVLWEMTSR CNAVGEVKDY EPPFGSKVRE HPCVESMKDN VLRDRGRPEI |
| 501 | PSFWLNHQGI QMVCETLTEC WDHDPEARLT AQCVAERFSE LEHLDRLSGR |
| 551 | SCSEEKIPED GSLNTTK |

The signal peptide is indicated by a single underline and the extracellular domain is indicated in bold font.

A processed extracellular TGFBRII polypeptide sequence is as follows:

(SEQ ID NO: 43)

TIPPHVQKSVNNDMIVTDNNGAVKFPQLCKFCDVRFSTCDNQKSCMSNCS
ITSICEKPQEVCVAVWRKNDENITLETVCHDPKLPYHDFILEDAASPKCI
MKEKKKPGETFFMCSCSSDECNDNIIFSEEYNTSNPDLLLVIFQ

A nucleic acid sequence encoding TGFBRII precursor protein is shown below (SEQ ID NO: 44), corresponding to nucleotides 383-2083 of Genbank Reference Sequence NM_003242.5. The signal sequence is underlined.

(SEQ ID NO: 44)

ATGGGTCGGGGGCTGCTCAGGGGCCTGTGGCCGCTGCACATCGTCCTGTG
GACGCGTATCGCCAGCACGATCCCACCGCACGTTCAGAAGTCGGTTAATA
ACGACATGATAGTCACTGACAACAACGGTGCAGTCAAGTTTCCACAACTG
TGTAAATTTTGTGATGTGAGATTTTCCACCTGTGACAACCAGAAATCCTG
CATGAGCAACTGCAGCATCACCTCCATCTGTGAGAAGCCACAGGAAGTCT
GTGTGGCTGTATGGAGAAAGAATGACGAGAACATAACACTAGAGACAGTT
TGCCATGACCCCAAGCTCCCCTACCATGACTTTATTCTGGAAGATGCTGC
TTCTCCAAAGTGCATTATGAAGGAAAAAAAAAAGCCTGGTGAGACTTTCT
TCATGTGTTCCTGTAGCTCTGATGAGTGCAATGACAACATCATCTTCTCA
GAAGAATATAACACCAGCAATCCTGACTTGTTGCTAGTCATATTTCAAGT
GACAGGCATCAGCCTCCTGCCACCACTGGGAGTTGCCATATCTGTCATCA
TCATCTTCTACTGCTACCGCGTTAACCGGCAGCAGAAGCTGAGTTCAACC
TGGGAAACCGGCAAGACGCGGAAGCTCATGGAGTTCAGCGAGCACTGTGC
CATCATCCTGGAAGATGACCGCTCTGACATCAGCTCCACGTGTGCCAACA
ACATCAACCACAACACAGAGCTGCTGCCCATTGAGCTGGACACCCTGGTG
GGGAAAGGTCGCTTTGCTGAGGTCTATAAGGCCAAGCTGAAGCAGAACAC
TTCAGAGCAGTTTGAGACAGTGGCAGTCAAGATCTTTCCCTATGAGGAGT
ATGCCTCTTGGAAGACAGAGAAGGACATCTTCTCAGACATCAATCTGAAG
CATGAGAACATACTCCAGTTCCTGACGGCTGAGGAGCGGAAGACGGAGTT
GGGGAAACAATACTGGCTGATCACCGCCTTCCACGCCAAGGGCAACCTAC
AGGAGTACCTGACGCGGCATGTCATCAGCTGGGAGGACCTGCGCAAGCTG
GGCAGCTCCCTCGCCCGGGGGATTGCTCACCTCCACAGTGATCACACTCC
ATGTGGGAGGCCCAAGATGCCCATCGTGCACAGGGACCTCAAGAGCTCCA
ATATCCTCGTGAAGAACGACCTAACCTGCTGCCTGTGTGACTTTGGGCTT
TCCCTGCGTCTGGACCCTACTCTGTCTGTGGATGACCTGGCTAACAGTGG
GCAGGTGGGAACTGCAAGATACATGGCTCCAGAAGTCCTAGAATCCAGGA
TGAATTTGGAGAATGTTGAGTCCTTCAAGCAGACCGATGTCTACTCCATG
GCTCTGGTGCTCTGGGAAATGACATCTCGCTGTAATGCAGTGGGAGAAGT
AAAAGATTATGAGCCTCCATTTGGTTCCAAGGTGCGGGAGCACCCCTGTG
TCGAAAGCATGAAGGACAACGTGTTGAGAGATCGAGGGCGACCAGAAATT
CCCAGCTTCTGGCTCAACCACCAGGGCATCCAGATGGTGTGTGAGACGTT
GACTGAGTGCTGGGACCACGACCCAGAGGCCCGTCTCACAGCCCAGTGTG
TGGCAGAACGCTTCAGTGAGCTGGAGCATCTGGACAGGCTCTCGGGGAGG
AGCTGCTCGGAGGAGAAGATTCCTGAAGACGGCTCCCTAAACACTACCAA
A

A nucleic acid sequence encoding a processed extracellular TGFBRII polypeptide is as follows:

(SEQ ID NO: 45)

ACGATCCCACCGCACGTTCAGAAGTCGGTTAATAACGACATGATAGTCAC
TGACAACAACGGTGCAGTCAAGTTTCCACAACTGTGTAAATTTTGTGATG
TGAGATTTTCCACCTGTGACAACCAGAAATCCTGCATGAGCAACTGCAGC
ATCACCTCCATCTGTGAGAAGCCACAGGAAGTCTGTGTGGCTGTATGGAG
AAAGAATGACGAGAACATAACACTAGAGACAGTTTGCCATGACCCCAAGC
TCCCCTACCATGACTTTATTCTGGAAGATGCTGCTTCTCCAAAGTGCATT
ATGAAGGAAAAAAAAAAGCCTGGTGAGACTTTCTTCATGTGTTCCTGTAG
CTCTGATGAGTGCAATGACAACATCATCTTCTCAGAAGAATATAACACCA
GCAATCCTGACTTGTTGCTAGTCATATTTCAA

An alternative isoform of TGFBRII, isoform A (NP_001020018.1), is as follows:

| (SEQ ID NO: 67) | |
|---|---|
| 1 | MGRGLLRGLW PLHIVLWTRI ASTIPPHVQK SDVEMEAQKD EIICPSCNRT |
| 51 | AHPLRHINND MIVTDNNGAV KFPQLCKFCD VRFSTCDNQK SCMSNCSITS |
| 101 | ICEKPQEVCV AVWRKNDENI TLETVCHDPK LPYHDFILED AASPKCIMKE |
| 151 | KKKPGETFFM CSCSSDECND NIIFSEEYNT SNPDLLLVIF QVTGISLLPP |
| 201 | LGVAISVIII FYCYRVNRQQ KLSSTWETGK TRKLMEFSEH CAIILEDDRS |
| 251 | DISSTCANNI NHNTELLPIE LDTLVGKGRF AEVYKAKLKQ NTSEQFETVA |
| 301 | VKIFPYEEYA SWKTEKDIFS DINLKHENIL QFLTAEERKT ELGKQYWLIT |
| 351 | AFHAKGNLQE YLTRHVISWE DLRKLGSSLA RGIAHLHSDH TPCGRPKMPI |
| 401 | VHRDLKSSNI LVKNDLTCCL CDFGLSLRLD PTLSVDDLAN SGQVGTARYM |
| 451 | APEVLESRMN LENVESFKQT DVYSMALVLW EMTSRCNAVG EVKDYEPPFG |
| 501 | SKVREHPCVE SMKDNVLRDR GRPEIPSFWL NHQGIQMVCE TLTECWDHDP |
| 551 | EARLTAQCVA ERFSELEHLD RLSGRSCSEE KIPEDGSLNT TK |

The signal peptide is indicated by a single underline and the extracellular domain is indicated in bold font.

A processed extracellular TGFBRII polypeptide sequence (isoform A) is as follows:

(SEQ ID NO: 68)

TIPPHVQKSDVEMEAQKDEIICPSCNRTAHPLRHINNDMIVTDNNGAVKF
PQLCKFCDVRFSTCDNQKSCMSNCSITSICEKPQEVCVAVWRKNDENITL
ETVCHDPKLPYHDFILEDAASPKCIMKEKKKPGETFFMCSCSSDECNDNI
IFSEEYNTSNPDLLLVIFQ

A nucleic acid sequence encoding the TGFBRII precursor protein (isoform A) is shown below (SEQ ID NO: 69), corresponding to nucleotides 383-2158 of Genbank Reference Sequence NM_001024847.2. The signal sequence is underlined.

(SEQ ID NO: 69)

ATGGGTCGGGGGCTGCTCAGGGGCCTGTGGCCGCTGCACATCGTCCTGTG
GACGCGTATCGCCAGCACGATCCCACCGCACGTTCAGAAGTCGGATGTGG
AAATGGAGGCCCAGAAAGATGAAATCATCTGCCCCAGCTGTAATAGGACT
GCCCATCCACTGAGACATATTAATAACGACATGATAGTCACTGACAACAA
CGGTGCAGTCAAGTTTCCACAACTGTGTAAATTTTGTGATGTGAGATTTT
CCACCTGTGACAACCAGAAATCCTGCATGAGCAACTGCAGCATCACCTCC
ATCTGTGAGAAGCCACAGGAAGTCTGTGTGGCTGTATGGAGAAAGAATGA
CGAGAACATAACACTAGAGACAGTTTGCCATGACCCCAAGCTCCCCTACC
ATGACTTTATTCTGGAAGATGCTGCTTCTCCAAAGTGCATTATGAAGGAA
AAAAAAAAGCCTGGTGAGACTTTCTTCATGTGTTCCTGTAGCTCTGATGA
GTGCAATGACAACATCATCTTCTCAGAAGAATATAACACCAGCAATCCTG
ACTTGTTGCTAGTCATATTTCAAGTGACAGGCATCAGCCTCCTGCCACCA
CTGGGAGTTGCCATATCTGTCATCATCATCTTCTACTGCTACCGCGTTAA
CCGGCAGCAGAAGCTGAGTTCAACCTGGGAAACCGGCAAGACGCGGAAGC
TCATGGAGTTCAGCGAGCACTGTGCCATCATCCTGGAAGATGACCGCTCT
GACATCAGCTCCACGTGTGCCAACAACATCAACCACAACACAGAGCTGCT
GCCCATTGAGCTGGACACCCTGGTGGGGAAAGGTCGCTTTGCTGAGGTCT
ATAAGGCCAAGCTGAAGCAGAACACTTCAGAGCAGTTTGAGACAGTGGCA
GTCAAGATCTTTCCCTATGAGGAGTATGCCTCTTGGAAGACAGAGAAGGA
CATCTTCTCAGACATCAATCTGAAGCATGAGAACATACTCCAGTTCCTGA
CGGCTGAGGAGCGGAAGACGGAGTTGGGGAAACAATACTGGCTGATCACC
GCCTTCCACGCCAAGGGCAACCTACAGGAGTACCTGACGCGGCATGTCAT
CAGCTGGGAGGACCTGCGCAAGCTGGGCAGCTCCCTCGCCCGGGGGATTG
CTCACCTCCACAGTGATCACACTCCATGTGGGAGGCCCAAGATGCCCATC
GTGCACAGGGACCTCAAGAGCTCCAATATCCTCGTGAAGAACGACCTAAC
CTGCTGCCTGTGTGACTTTGGGCTTTCCCTGCGTCTGGACCCTACTCTGT
CTGTGGATGACCTGGCTAACAGTGGGCAGGTGGGAACTGCAAGATACATG
GCTCCAGAAGTCCTAGAATCCAGGATGAATTTGGAGAATGTTGAGTCCTT
CAAGCAGACCGATGTCTACTCCATGGCTCTGGTGCTCTGGGAAATGACAT
CTCGCTGTAATGCAGTGGGAGAAGTAAAAGATTATGAGCCTCCATTTGGT
TCCAAGGTGCGGGAGCACCCCTGTGTCGAAAGCATGAAGGACAACGTGTT
GAGAGATCGAGGGCGACCAGAAATTCCCAGCTTCTGGCTCAACCACCAGG
GCATCCAGATGGTGTGTGAGACGTTGACTGAGTGCTGGGACCACGACCCA
GAGGCCCGTCTCACAGCCCAGTGTGTGGCAGAACGCTTCAGTGAGCTGGA
GCATCTGGACAGGCTCTCGGGGAGGAGCTGCTCGGAGGAGAAGATTCCTG
AAGACGGCTCCCTAAACACTACCAAA

A nucleic acid sequence encoding an processed extracellular TGFBRII polypeptide (isoform A) is as follows:

(SEQ ID NO: 70)

ACGATCCCACCGCACGTTCAGAAGTCGGATGTGGAAATGGAGGCCCAGAA
AGATGAAATCATCTGCCCCAGCTGTAATAGGACTGCCCATCCACTGAGAC
ATATTAATAACGACATGATAGTCACTGACAACAACGGTGCAGTCAAGTTT
CCACAACTGTGTAAATTTTGTGATGTGAGATTTTCCACCTGTGACAACCA
GAAATCCTGCATGAGCAACTGCAGCATCACCTCCATCTGTGAGAAGCCAC
AGGAAGTCTGTGTGGCTGTATGGAGAAAGAATGACGAGAACATAACACTA
GAGACAGTTTGCCATGACCCCAAGCTCCCCTACCATGACTTTATTCTGGA
AGATGCTGCTTCTCCAAAGTGCATTATGAAGGAAAAAAAAAAGCCTGGTG
AGACTTTCTTCATGTGTTCCTGTAGCTCTGATGAGTGCAATGACAACATC
ATCTTCTCAGAAGAATATAACACCAGCAATCCTGACTTGTTGCTAGTCAT
ATTTCAA.

Either of the foregoing TGFBRII isoforms (SEQ ID NOs: 42, 43, 67, and 68) could incorporate an insertion of 36 amino acids (SEQ ID NO: 95) between the pair of glutamate residues (positions 151 and 152 of SEQ ID NO: 42; positions 129 and 130 of SEQ ID NO: 43; positions 176 and 177 of SEQ ID NO: 67; or positions 154 and 155 of SEQ ID NO: 68) located near the C-terminus of the TGFBRII ECD, as occurs naturally in the TGFBRII isoform C (Konrad et al., BMC Genomics 8:318, 2007).

(SEQ ID NO: 95)

GRCKIRHIGS NNRLQRSTCQ NTGWESAHVM KTPGFR

In certain embodiments, the disclosure relates to heteromultimers that comprise at least one TGFBRII polypeptide, which includes fragments, functional variants, and modified forms thereof. Preferably, TGFBRII polypeptides for use in accordance with the disclosure (e.g., heteromultimers comprising a TGFBRII polypeptide and uses thereof) are soluble (e.g., an extracellular domain of TGFBRII). In other preferred embodiments, TGFBRII polypeptides for use in accordance with the disclosure bind to and/or inhibit (antagonize) activity (e.g., Smad signaling) of one or more TGF-beta superfamily ligands. In some embodiments, heteromultimers of the disclosure comprise at least one TGFBRII polypeptide that is at least 70%, 75%, 80%, 85%, 90%, 91%, 92%, 93%, 94%, 95%, 96%, 97%, 98%, 99%, or 100% identical to the amino acid sequence of SEQ ID NOs: 42, 43, 67, or 68, with or without insertion of SEQ ID NO: 95 as described above. In some embodiments, heteromultimers of the disclosure comprise at least one TGFBRII polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 23-51 (e.g., amino acid residues 23, 24, 25, 26, 27, 28, 29, 30, 31, 32, 33, 34, 35, 36, 37, 38, 39, 40, 41, 42, 43, 44, 45, 46, 47, 48, 49, 50, or 51) of SEQ ID NO: 42, and ends at any one of amino acids 143-166 (e.g., amino acid residues 143, 144, 145, 146, 147, 148, 149, 150, 151, 152, 153, 154, 155, 156, 157, 158, 159, 160, 161, 162, 163, 164, 165, or 166) of SEQ ID NO: 42. In some embodiments, heteromultimers of the disclosure comprise at least one TGFBRII polypeptide that is at least 70%, 75%, 80%, 85%, 90%, 91%, 92%, 93%, 94%, 95%, 96%, 97%, 98%, 99%, or 100% identical to amino acids of 23-143 of SEQ ID NO: 42. In some embodiments, heteromultimers of the disclosure comprise at least one TGFBRII polypeptide that is at least 70%, 75%, 80%, 85%, 90%, 91%, 92%, 93%, 94%, 95%, 96%, 97%, 98%, 99%, or 100% identical to amino acids of 51-143 of SEQ ID NO: 42. In some embodiments, heteromultimers of the disclosure comprise at least one TGFBRII polypeptide that is at least 70%, 75%, 80%, 85%, 90%, 91%, 92%, 93%, 94%, 95%, 96%, 97%, 98%, 99%, or 100% identical to amino acids of 23-166 of SEQ ID NO: 42. In some embodiments, heteromultimers of the disclosure comprise at least one TGFBRII polypeptide that is at least 70%, 75%, 80%, 85%, 90%, 91%, 92%, 93%, 94%, 95%, 96%, 97%, 98%, 99%, or 100% identical to amino acids of 51-166 of SEQ ID NO: 42. In some embodiments, heteromultimers of the disclosure comprise at least one TGFBRII polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 23-44 (e.g., amino acid residues 23, 24, 25, 26, 27, 28, 29, 30, 31, 32, 33, 34, 35, 36, 37, 38, 39, 40, 41, 42, 43, or 44) of SEQ ID NO: 67, and ends at any one of amino acids 168-191 (e.g., amino acid residues 168, 169, 170, 171, 172, 173, 174, 175, 176, 177, 178, 179, 180, 181, 182, 183, 184, 185, 186, 187, 188, 189, 190, or 191) of SEQ ID NO: 67. In some embodiments, heteromultimers of the disclosure comprise at least one TGFBRII polypeptide that is at least 70%, 75%, 80%, 85%, 90%, 91%, 92%, 93%, 94%, 95%, 96%, 97%, 98%, 99%, or 100% identical to amino acids of 23-168 of SEQ ID NO: 67. In some embodiments, heteromultimers of the disclosure comprise at least one TGFBRII polypeptide that is at least 70%, 75%, 80%, 85%, 90%, 91%, 92%, 93%, 94%, 95%, 96%, 97%, 98%, 99%, or 100% identical to amino acids of 23-191 of SEQ ID NO: 67. In some embodiments, heteromultimers of the disclosure comprise at least one TGFBRII polypeptide that is at least 70%, 75%, 80%, 85%, 90%, 91%, 92%, 93%, 94%, 95%, 96%, 97%, 98%, 99%, or 100% identical to amino acids of 44-168 of SEQ ID NO: 67. In some embodiments, heteromultimers of the disclosure comprise at least one TGFBRII polypeptide that is at least 70%, 75%, 80%, 85%, 90%, 91%, 92%, 93%, 94%, 95%, 96%, 97%, 98%, 99%, or 100% identical to amino acids of 44-191 of SEQ ID NO: 67.

In certain aspects, the present disclosure relates to heteromultimers that comprise a BMPRII polypeptide. As used herein, the term “BMPRII” refers to a family of bone morphogenetic protein receptor type II (BMPRII) proteins from any species and variants derived from such BMPRII proteins by mutagenesis or other modification. Reference to BMPRII herein is understood to be a reference to any one of the currently identified forms. Members of the BMPRII family are generally transmembrane proteins, composed of a ligand-binding extracellular domain with a cysteine-rich region, a transmembrane domain, and a cytoplasmic domain with predicted serine/threonine kinase activity.

The term “BMPRII polypeptide” includes polypeptides comprising any naturally occurring polypeptide of a BMPRII family member as well as any variants thereof (including mutants, fragments, fusions, and peptidomimetic forms) that retain a useful activity.

A human BMPRII precursor protein sequence (NCBI Ref Seq NP_001195.2) is as follows:

| (SEQ ID NO: 46) | |
|---|---|
| 1 | MTSSLQRPWR VPWLPWTILL VSTAAASQNQ ERLCAFKDPY QQDLGIGESR |
| 51 | ISHENGTILC SKGSTCYGLW EKSKGDINLV KQGCWSHIGD PQECHYEECV |
| 101 | VTTTPPSIQN GTYRFCCCST DLCNVNFTEN FPPPDTTPLS PPHSFNRDET |
| 151 | IIIALASVSV LAVLIVALCF GYRMLTGDRK QGLHSMNMME AAASEPSLDL |
| 201 | DNLKLLELIG RGRYGAVYKG SLDERPVAVK VFSFANRQNF INEKNIYRVP |
| 251 | LMEHDNIARF IVGDERVTAD GRMEYLLVME YYPNGSLCKY LSLHTSDWVS |
| 301 | SCRLAHSVTR GLAYLHTELP RGDHYKPAIS HRDLNSRNVL VKNDGTCVIS |
| 351 | DFGLSMRLTG NRLVRPGEED NAAISEVGTI RYMAPEVLEG AVNLRDCESA |
| 401 | LKQVDMYALG LIYWEIFMRC TDLFPGESVP EYQMAFQTEV GNHPTFEDMQ |
| 451 | VLVSREKQRP KFPEAWKENS LAVRSLKETI EDCWDQDAEA RLTAQCAEER |
| 501 | MAELMMIWER NKSVSPTVNP MSTAMQNERN LSHNRRVPKI GPYPDYSSSS |
| 551 | YIEDSIHHTD SIVKNISSEH SMSSTPLTIG EKNRNSINYE RQQAQARIPS |
| 601 | PETSVTSLST NTTTTNTTGL TPSTGMTTIS EMPYPDETNL HTTNVAQSIG |
| 651 | PTPVCLQLTE EDLETNKLDP KEVDKNLKES SDENLMEHSL KQFSGPDPLS |
| 701 | STSSSLLYPL IKLAVEATGQ QDFTQTANGQ ACLIPDVLPT QIYPLPKQQN |
| 751 | LPKRPTSLPL NTKNSTKEPR LKFGSKHKSN LKQVETGVAK MNTINAAEPH |
| 801 | VVTVTMNGVA GRNHSVNSHA ATTQYANGTV LSGQTTNIVT HRAQEMLQNQ |
| 851 | FIGEDTRLNI NSSPDEHEPL LRREQQAGHD EGVLDRLVDR RERPLEGGRT |
| 901 | NSNNNNSNPC SEQDVLAQGV PSTAADPGPS KPRRAQRPNS LDLSATNVLD |
| 951 | GSSIQIGEST QDGKSGSGEK IKKRVKTPYS LKRWRPSTWV ISTESLDCEV |
| 1001 | NNNGSNRAVH SKSSTAVYLA EGGTATTMVS KDIGMNCL |

The signal peptide is indicated by a single underline and the extracellular domain is indicated in bold font.

A processed extracellular BMPRII polypeptide sequence is as follows:

(SEQ ID NO: 47)

SQNQERLCAFKDPYQQDLGIGESRISHENGTILCSKGSTCYGLWEKSKGD
INLVKQGCWSHIGDPQECHYEECVVTTTPPSIQNGTYRFCCCSTDLCNVN
FTENFPPPDTTPLSPPHSFNRDET

A nucleic acid sequence encoding BMPRII precursor protein is shown below (SEQ ID NO: 48), as follows nucleotides 1149-4262 of Genbank Reference Sequence NM_001204.6. The signal sequence is underlined.

(SEQ ID NO: 48)

ATGACTTCCTCGCTGCAGCGGCCCTGGCGGGTGCCCTGGCTACCATGGAC
CATCCTGCTGGTCAGCACTGCGGCTGCTTCGCAGAATCAAGAACGGCTAT
GTGCGTTTAAAGATCCGTATCAGCAAGACCTTGGGATAGGTGAGAGTAGA
ATCTCTCATGAAAATGGGACAATATTATGCTCGAAAGGTAGCACCTGCTA
TGGCCTTTGGGAGAAATCAAAAGGGGACATAAATCTTGTAAAACAAGGAT
GTTGGTCTCACATTGGAGATCCCCAAGAGTGTCACTATGAAGAATGTGTA
GTAACTACCACTCCTCCCTCAATTCAGAATGGAACATACCGTTTCTGCTG
TTGTAGCACAGATTTATGTAATGTCAACTTTACTGAGAATTTTCCACCTC
CTGACACAACACCACTCAGTCCACCTCATTCATTTAACCGAGATGAGACA
ATAATCATTGCTTTGGCATCAGTCTCTGTATTAGCTGTTTTGATAGTTGC
CTTATGCTTTGGATACAGAATGTTGACAGGAGACCGTAAACAAGGTCTTC
ACAGTATGAACATGATGGAGGCAGCAGCATCCGAACCCTCTCTTGATCTA
GATAATCTGAAACTGTTGGAGCTGATTGGCCGAGGTCGATATGGAGCAGT
ATATAAAGGCTCCTTGGATGAGCGTCCAGTTGCTGTAAAAGTGTTTTCCT
TTGCAAACCGTCAGAATTTTATCAACGAAAAGAACATTTACAGAGTGCCT
TTGATGGAACATGACAACATTGCCCGCTTTATAGTTGGAGATGAGAGAGT
CACTGCAGATGGACGCATGGAATATTTGCTTGTGATGGAGTACTATCCCA
ATGGATCTTTATGCAAGTATTTAAGTCTCCACACAAGTGACTGGGTAAGC
TCTTGCCGTCTTGCTCATTCTGTTACTAGAGGACTGGCTTATCTTCACAC
AGAATTACCACGAGGAGATCATTATAAACCTGCAATTTCCCATCGAGATT
TAAACAGCAGAAATGTCCTAGTGAAAAATGATGGAACCTGTGTTATTAGT
GACTTTGGACTGTCCATGAGGCTGACTGGAAATAGACTGGTGCGCCCAGG
GGAGGAAGATAATGCAGCCATAAGCGAGGTTGGCACTATCAGATATATGG
CACCAGAAGTGCTAGAAGGAGCTGTGAACTTGAGGGACTGTGAATCAGCT
TTGAAACAAGTAGACATGTATGCTCTTGGACTAATCTATTGGGAGATATT
TATGAGATGTACAGACCTCTTCCCAGGGGAATCCGTACCAGAGTACCAGA
TGGCTTTTCAGACAGAGGTTGGAAACCATCCCACTTTTGAGGATATGCAG
GTTCTCGTGTCTAGGGAAAAACAGAGACCCAAGTTCCCAGAAGCCTGGAA
AGAAAATAGCCTGGCAGTGAGGTCACTCAAGGAGACAATCGAAGACTGTT
GGGACCAGGATGCAGAGGCTCGGCTTACTGCACAGTGTGCTGAGGAAAGG
ATGGCTGAACTTATGATGATTTGGGAAAGAAACAAATCTGTGAGCCCAAC
AGTCAATCCAATGTCTACTGCTATGCAGAATGAACGCAACCTGTCACATA
ATAGGCGTGTGCCAAAAATTGGTCCTTATCCAGATTATTCTTCCTCCTCA
TACATTGAAGACTCTATCCATCATACTGACAGCATCGTGAAGAATATTTC
CTCTGAGCATTCTATGTCCAGCACACCTTTGACTATAGGGGAAAAAAACC
GAAATTCAATTAACTATGAACGACAGCAAGCACAAGCTCGAATCCCCAGC
CCTGAAACAAGTGTCACCAGCCTCTCCACCAACACAACAACCACAAACAC
CACAGGACTCACGCCAAGTACTGGCATGACTACTATATCTGAGATGCCAT
ACCCAGATGAAACAAATCTGCATACCACAAATGTTGCACAGTCAATTGGG
CCAACCCCTGTCTGCTTACAGCTGACAGAAGAAGACTTGGAAACCAACAA
GCTAGACCCAAAAGAAGTTGATAAGAACCTCAAGGAAAGCTCTGATGAGA
ATCTCATGGAGCACTCTCTTAAACAGTTCAGTGGCCCAGACCCACTGAGC
AGTACTAGTTCTAGCTTGCTTTACCCACTCATAAAACTTGCAGTAGAAGC
AACTGGACAGCAGGACTTCACACAGACTGCAAATGGCCAAGCATGTTTGA
TTCCTGATGTTCTGCCTACTCAGATCTATCCTCTCCCCAAGCAGCAGAAC
CTTCCCAAGAGACCTACTAGTTTGCCTTTGAACACCAAAAATTCAACAAA
AGAGCCCCGGCTAAAATTTGGCAGCAAGCACAAATCAAACTTGAAACAAG
TCGAAACTGGAGTTGCCAAGATGAATACAATCAATGCAGCAGAACCTCAT
GTGGTGACAGTCACCATGAATGGTGTGGCAGGTAGAAACCACAGTGTTAA
CTCCCATGCTGCCACAACCCAATATGCCAATGGGACAGTACTATCTGGCC
AAACAACCAACATAGTGACACATAGGGCCCAAGAAATGTTGCAGAATCAG
TTTATTGGTGAGGACACCCGGCTGAATATTAATTCCAGTCCTGATGAGCA
TGAGCCTTTACTGAGACGAGAGCAACAAGCTGGCCATGATGAAGGTGTTC
TGGATCGTCTTGTGGACAGGAGGGAACGGCCACTAGAAGGTGGCCGAACT
AATTCCAATAACAACAACAGCAATCCATGTTCAGAACAAGATGTTCTTGC
ACAGGGTGTTCCAAGCACAGCAGCAGATCCTGGGCCATCAAAGCCCAGAA
GAGCACAGAGGCCTAATTCTCTGGATCTTTCAGCCACAAATGTCCTGGAT
GGCAGCAGTATACAGATAGGTGAGTCAACACAAGATGGCAAATCAGGATC
AGGTGAAAAGATCAAGAAACGTGTGAAAACTCCCTATTCTCTTAAGCGGT
GGCGCCCCTCCACCTGGGTCATCTCCACTGAATCGCTGGACTGTGAAGTC
AACAATAATGGCAGTAACAGGGCAGTTCATTCCAAATCCAGCACTGCTGT
TTACCTTGCAGAAGGAGGCACTGCTACAACCATGGTGTCTAAAGATATAG
GAATGAACTGTCTG

A nucleic acid sequence encoding an extracellular BMPRII polypeptide is as follows:

(SEQ ID NO: 49)

TCGCAGAATCAAGAACGGCTATGTGCGTTTAAAGATCCGTATCAGCAAGA
CCTTGGGATAGGTGAGAGTAGAATCTCTCATGAAAATGGGACAATATTAT
GCTCGAAAGGTAGCACCTGCTATGGCCTTTGGGAGAAATCAAAAGGGGAC
ATAAATCTTGTAAAACAAGGATGTTGGTCTCACATTGGAGATCCCCAAGA
GTGTCACTATGAAGAATGTGTAGTAACTACCACTCCTCCCTCAATTCAGA
ATGGAACATACCGTTTCTGCTGTTGTAGCACAGATTTATGTAATGTCAAC
TTTACTGAGAATTTTCCACCTCCTGACACAACACCACTCAGTCCACCTCA
TTCATTTAACCGAGATGAGACA

An alternative isoform of BMPRII, isoform 2 (GenBank: AAA86519.1) is as follows:

| (SEQ ID NO: 71) | |
|---|---|
| 1 | MTSSLQRPWR VPWLPWTILL VSTAAASQNQ ERLCAFKDPY QQDLGIGESR |
| 51 | ISHENGTILC SKGSTCYGLW EKSKGDINLV KQGCWSHIGD PQECHYEECV |
| 101 | VTTTPPSIQN GTYRFCCCST DLCNVNFTEN FPPPDTTPLS PPHSFNRDET |
| 151 | IIIALASVSV LAVLIVALCF GYRMLTGDRK QGLHSMNMME AAASEPSLDL |
| 201 | DNLKLLELIG RGRYGAVYKG SLDERPVAVK VFSFANRQNF INEKNIYRVP |
| 251 | LMEHDNIARF IVGDERVTAD GRMEYLLVME YYPNGSLCKY LSLHTSDWVS |
| 301 | SCRLAHSVTR GLAYLHTELP RGDHYKPAIS HRDLNSRNVL VKNDGTCVIS |
| 351 | DFGLSMRLTG NRLVRPGEED NAAISEVGTI RYMAPEVLEG AVNLRDCESA |
| 401 | LKQVDMYALG LIYWEIFMRC TDLFPGESVP EYQMAFQTEV GNHPTFEDMQ |
| 451 | VLVSREKQRP KFPEAWKENS LAVRSLKETI EDCWDQDAEA RLTAQCAEER |
| 501 | MAELMMIWER NKSVSPTVNP MSTAMQNERR |

The signal peptide is indicated by a single underline and the extracellular domain is indicated in bold font.

A processed extracellular BMPRII polypeptide sequence (isoform 2) is as follows:

(SEQ ID NO: 72)

SQNQERLCAFKDPYQQDLGIGESRISHENGTILCSKGSTCYGLWEKSKGD
INLVKQGCWSHIGDPQECHYEECVVTTTPPSIQNGTYRFCCCSTDLCNVN
FTENFPPPDTTPLSPPHSFNRDET

A nucleic acid sequence encoding human BMPRII precursor protein (isoform 2) is shown below (SEQ ID NO: 73), corresponding to nucleotides 163-1752 of Genbank Reference Sequence U25110.1. The signal sequence is underlined.

(SEQ ID NO: 73)

ATGACTTCCTCGCTGCAGCGGCCCTGGCGGGTGCCCTGGCTACCATGGAC
CATCCTGCTGGTCAGCACTGCGGCTGCTTCGCAGAATCAAGAACGGCTAT
GTGCGTTTAAAGATCCGTATCAGCAAGACCTTGGGATAGGTGAGAGTAGA
ATCTCTCATGAAAATGGGACAATATTATGCTCGAAAGGTAGCACCTGCTA
TGGCCTTTGGGAGAAATCAAAAGGGGACATAAATCTTGTAAAACAAGGAT
GTTGGTCTCACATTGGAGATCCCCAAGAGTGTCACTATGAAGAATGTGTA
GTAACTACCACTCCTCCCTCAATTCAGAATGGAACATACCGTTTCTGCTG
TTGTAGCACAGATTTATGTAATGTCAACTTTACTGAGAATTTTCCACCTC
CTGACACAACACCACTCAGTCCACCTCATTCATTTAACCGAGATGAGACA
ATAATCATTGCTTTGGCATCAGTCTCTGTATTAGCTGTTTTGATAGTTGC
CTTATGCTTTGGATACAGAATGTTGACAGGAGACCGTAAACAAGGTCTTC
ACAGTATGAACATGATGGAGGCAGCAGCATCCGAACCCTCTCTTGATCTA
GATAATCTGAAACTGTTGGAGCTGATTGGCCGAGGTCGATATGGAGCAGT
ATATAAAGGCTCCTTGGATGAGCGTCCAGTTGCTGTAAAAGTGTTTTCCT
TTGCAAACCGTCAGAATTTTATCAACGAAAAGAACATTTACAGAGTGCCT
TTGATGGAACATGACAACATTGCCCGCTTTATAGTTGGAGATGAGAGAGT
CACTGCAGATGGACGCATGGAATATTTGCTTGTGATGGAGTACTATCCCA
ATGGATCTTTATGCAAGTATTTAAGTCTCCACACAAGTGACTGGGTAAGC
TCTTGCCGTCTTGCTCATTCTGTTACTAGAGGACTGGCTTATCTTCACAC
AGAATTACCACGAGGAGATCATTATAAACCTGCAATTTCCCATCGAGATT
TAAACAGCAGAAATGTCCTAGTGAAAAATGATGGAACCTGTGTTATTAGT
GACTTTGGACTGTCCATGAGGCTGACTGGAAATAGACTGGTGCGCCCAGG
GGAGGAAGATAATGCAGCCATAAGCGAGGTTGGCACTATCAGATATATGG
CACCAGAAGTGCTAGAAGGAGCTGTGAACTTGAGGGACTGTGAATCAGCT
TTGAAACAAGTAGACATGTATGCTCTTGGACTAATCTATTGGGAGATATT
TATGAGATGTACAGACCTCTTCCCAGGGGAATCCGTACCAGAGTACCAGA
TGGCTTTTCAGACAGAGGTTGGAAACCATCCCACTTTTGAGGATATGCAG
GTTCTCGTGTCTAGGGAAAAACAGAGACCCAAGTTCCCAGAAGCCTGGAA
AGAAAATAGCCTGGCAGTGAGGTCACTCAAGGAGACAATCGAAGACTGTT
GGGACCAGGATGCAGAGGCTCGGCTTACTGCACAGTGTGCTGAGGAAAGG
ATGGCTGAACTTATGATGATTTGGGAAAGAAACAAATCTGTGAGCCCAAC
AGTCAATCCAATGTCTACTGCTATGCAGAATGAACGTAGG

A nucleic acid sequence encoding an extracellular BMPRII polypeptide (isoform 2) is as follows:

(SEQ ID NO: 74)

TCGCAGAATCAAGAACGGCTATGTGCGTTTAAAGATCCGTATCAGCAAGA
CCTTGGGATAGGTGAGAGTAGAATCTCTCATGAAAATGGGACAATATTAT
GCTCGAAAGGTAGCACCTGCTATGGCCTTTGGGAGAAATCAAAAGGGGAC
ATAAATCTTGTAAAACAAGGATGTTGGTCTCACATTGGAGATCCCCAAGA
GTGTCACTATGAAGAATGTGTAGTAACTACCACTCCTCCCTCAATTCAGA
ATGGAACATACCGTTTCTGCTGTTGTAGCACAGATTTATGTAATGTCAAC
TTTACTGAGAATTTTCCACCTCCTGACACAACACCACTCAGTCCACCTCA
TTCATTTAACCGAGATGAGACA

In certain embodiments, the disclosure relates to heteromultimers that comprise at least one BMPRII polypeptide, which includes fragments, functional variants, and modified forms thereof. Preferably, BMPRII polypeptides for use in accordance with the disclosure (e.g., heteromultimers comprising a BMPRII polypeptide and uses thereof) are soluble (e.g., an extracellular domain of BMPRII). In other preferred embodiments, BMPRII polypeptides for use in accordance with the disclosure bind to and/or inhibit (antagonize) activity (e.g., Smad signaling) of one or more TGF-beta superfamily ligands. In some embodiments, heteromultimers of the disclosure comprise at least one BMPRII polypeptide that is at least 70%, 75%, 80%, 85%, 90%, 91%, 92%, 93%, 94%, 95%, 96%, 97%, 98%, 99%, or 100% identical to the amino acid sequence of SEQ ID NO: 46, 47, 71, or 72. In some embodiments, heteromultimers of the disclosure comprise at least one BMPRII polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 27-34 (e.g., amino acid residues 27, 28, 29, 30, 31, 32, 33, or 34) of SEQ ID NO: 46, and ends at any one of amino acids 123-150 (e.g., amino acid residues 123, 124, 125, 126, 127, 128, 129, 130, 131, 132, 133, 134, 135, 136, 137, 138, 139, 140, 141, 142, 143, 144, 145, 146, 147, 148, 149, or 150) of SEQ ID NO: 46. In some embodiments, heteromultimers of the disclosure comprise at least one BMPRII polypeptide that is at least 70%, 75%, 80%, 85%, 90%, 91%, 92%, 93%, 94%, 95%, 96%, 97%, 98%, 99%, or 100% identical to amino acids of 27-123 of SEQ ID NO: 46. In some embodiments, heteromultimers of the disclosure comprise at least one BMPRII polypeptide that is at least 70%, 75%, 80%, 85%, 90%, 91%, 92%, 93%, 94%, 95%, 96%, 97%, 98%, 99%, or 100% identical to amino acids of 27-150 of SEQ ID NO: 46. In some embodiments, heteromultimers of the disclosure comprise at least one BMPRII polypeptide that is at least 70%, 75%, 80%, 85%, 90%, 91%, 92%, 93%, 94%, 95%, 96%, 97%, 98%, 99%, or 100% identical to amino acids of 34-123 of SEQ ID NO: 46. In some embodiments, heteromultimers of the disclosure comprise at least one BMPRII polypeptide that is at least 70%, 75%, 80%, 85%, 90%, 91%, 92%, 93%, 94%, 95%, 96%, 97%, 98%, 99%, or 100% identical to amino acids of 34-150 of SEQ ID NO: 46. In some embodiments, heteromultimers of the disclosure comprise at least one BMPRII polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 27-34 (e.g., amino acid residues 27, 28, 29, 30, 31, 32, 33, or 34) of SEQ ID NO: 71, and ends at any one of amino acids 123-150 (e.g., amino acid residues 123, 124, 125, 126, 127, 128, 129, 130, 131, 132, 133, 134, 135, 136, 137, 138, 139, 140, 141, 142, 143, 144, 145, 146, 147, 148, 149, or 150) of SEQ ID NO: 71. In some embodiments, heteromultimers of the disclosure comprise at least one BMPRII polypeptide that is at least 70%, 75%, 80%, 85%, 90%, 91%, 92%, 93%, 94%, 95%, 96%, 97%, 98%, 99%, or 100% identical to amino acids of 27-123 of SEQ ID NO: 71. In some embodiments, heteromultimers of the disclosure comprise at least one BMPRII polypeptide that is at least 70%, 75%, 80%, 85%, 90%, 91%, 92%, 93%, 94%, 95%, 96%, 97%, 98%, 99%, or 100% identical to amino acids of 27-150 of SEQ ID NO: 71. In some embodiments, heteromultimers of the disclosure comprise at least one BMPRII polypeptide that is at least 70%, 75%, 80%, 85%, 90%, 91%, 92%, 93%, 94%, 95%, 96%, 97%, 98%, 99%, or 100% identical to amino acids of 34-123 of SEQ ID NO: 71. In some embodiments, heteromultimers of the disclosure comprise at least one BMPRII polypeptide that is at least 70%, 75%, 80%, 85%, 90%, 91%, 92%, 93%, 94%, 95%, 96%, 97%, 98%, 99%, or 100% identical to amino acids of 34-150 of SEQ ID NO: 71.

In certain aspects, the present disclosure relates to heteromultimers that comprise an MISRII polypeptide. As used herein, the term “MISRII” refers to a family of Müllerian inhibiting substance receptor type II (MISRII) proteins from any species and variants derived from such MISRII proteins by mutagenesis or other modification. Reference to MISRII herein is understood to be a reference to any one of the currently identified forms. Members of the MISRII family are generally transmembrane proteins, composed of a ligand-binding extracellular domain with a cysteine-rich region, a transmembrane domain, and a cytoplasmic domain with predicted serine/threonine kinase activity.

The term “MISRII polypeptide” includes polypeptides comprising any naturally occurring polypeptide of an MISRII family member as well as any variants thereof (including mutants, fragments, fusions, and peptidomimetic forms) that retain a useful activity.

The human MISRII isoform 1 precursor protein sequence (NCBI Ref Seq NP_065434.1) is as follows:

| (SEQ ID NO: 50) | |
|---|---|
| 1 | MLGSLGLWAL LPTAVEAPPN RRTCVFFEAP GVRGSTKTLG ELLDTGTELP |
| 51 | RAIRCLYSRC CFGIWNLTQD RAQVEMQGCR DSDEPGCESL HCDPSPRAHP |
| 101 | SPGSTLFTCS CGTDFCNANY SHLPPPGSPG TPGSQGPQAA PGESIWMALV |
| 151 | LLGLFLLLLL LLGSIILALL QRKNYRVRGE PVPEPRPDSG RDWSVELQEL |
| 201 | PELCFSQVIR EGGHAVVWAG QLQGKLVAIK AFPPRSVAQF QAERALYELP |
| 251 | GLQHDHIVRF ITASRGGPGR LLSGPLLVLE LHPKGSLCHY LTQYTSDWGS |
| 301 | SLRMALSLAQ GLAFLHEERW QNGQYKPGIA HRDLSSQNVL IREDGSCAIG |
| 351 | DLGLALVLPG LTQPPAWTPT QPQGPAAIME AGTQRYMAPE LLDKTLDLQD |
| 401 | WGMALRRADI YSLALLLWEI LSRCPDLRPD SSPPPFQLAY EAELGNTPTS |
| 451 | DELWALAVQE RRRPYIPSTW RCFATDPDGL RELLEDCWDA DPEARLTAEC |
| 501 | VQQRLAALAH PQESHPFPES CPRGCPPLCP EDCTSIPAPT ILPCRPQRSA |
| 551 | CHFSVQQGPC SRNPQPACTL SPV |

The signal peptide is indicated by a single underline and the extracellular domain is indicated in bold font.

A processed extracellular MISRII polypeptide sequence (isoform 1) is as follows:

(SEQ ID NO: 51)

PPNRRTCVFFEAPGVRGSTKTLGELLDTGTELPRAIRCLYSRCCFGIWNL
TQDRAQVEMQGCRDSDEPGCESLHCDPSPRAHPSPGSTLFTCSCGTDFCN
ANYSHLPPPGSPGTPGSQGPQAAPGESIWMAL

A nucleic acid sequence encoding the MISRII precursor protein is shown below (SEQ ID NO: 52), corresponding to nucleotides 81-1799 of Genbank Reference Sequence NM_020547.2. The signal sequence is underlined.

(SEQ ID NO: 52)

ATGCTAGGGTCTTTGGGGCTTTGGGCATTACTTCCCACAGCTGTGGAAGC
ACCCCCAAACAGGCGAACCTGTGTGTTCTTTGAGGCCCCTGGAGTGCGGG
GAAGCACAAAGACACTGGGAGAGCTGCTAGATACAGGCACAGAGCTCCCC
AGAGCTATCCGCTGCCTCTACAGCCGCTGCTGCTTTGGGATCTGGAACCT
GACCCAAGACCGGGCACAGGTGGAAATGCAAGGATGCCGAGACAGTGATG
AGCCAGGCTGTGAGTCCCTCCACTGTGACCCAAGTCCCCGAGCCCACCCC
AGCCCTGGCTCCACTCTCTTCACCTGCTCCTGTGGCACTGACTTCTGCAA
TGCCAATTACAGCCATCTGCCTCCTCCAGGGAGCCCTGGGACTCCTGGCT
CCCAGGGTCCCCAGGCTGCCCCAGGTGAGTCCATCTGGATGGCACTGGTG
CTGCTGGGGCTGTTCCTCCTCCTCCTGCTGCTGCTGGGCAGCATCATCTT
GGCCCTGCTACAGCGAAAGAACTACAGAGTGCGAGGTGAGCCAGTGCCAG
AGCCAAGGCCAGACTCAGGCAGGGACTGGAGTGTGGAGCTGCAGGAGCTG
CCTGAGCTGTGTTTCTCCCAGGTAATCCGGGAAGGAGGTCATGCAGTGGT
TTGGGCCGGGCAGCTGCAAGGAAAACTGGTTGCCATCAAGGCCTTCCCAC
CGAGGTCTGTGGCTCAGTTCCAAGCTGAGAGAGCATTGTACGAACTTCCA
GGCCTACAGCACGACCACATTGTCCGATTTATCACTGCCAGCCGGGGGGG
TCCTGGCCGCCTGCTCTCTGGGCCCCTGCTGGTACTGGAACTGCATCCCA
AGGGCTCCCTGTGCCACTACTTGACCCAGTACACCAGTGACTGGGGAAGT
TCCCTGCGGATGGCACTGTCCCTGGCCCAGGGCCTGGCATTTCTCCATGA
GGAGCGCTGGCAGAATGGCCAATATAAACCAGGTATTGCCCACCGAGATC
TGAGCAGCCAGAATGTGCTCATTCGGGAAGATGGATCGTGTGCCATTGGA
GACCTGGGCCTTGCCTTGGTGCTCCCTGGCCTCACTCAGCCCCCTGCCTG
GACCCCTACTCAACCACAAGGCCCAGCTGCCATCATGGAAGCTGGCACCC
AGAGGTACATGGCACCAGAGCTCTTGGACAAGACTCTGGACCTACAGGAT
TGGGGCATGGCCCTCCGACGAGCTGATATTTACTCTTTGGCTCTGCTCCT
GTGGGAGATACTGAGCCGCTGCCCAGATTTGAGGCCTGACAGCAGTCCAC
CACCCTTCCAACTGGCCTATGAGGCAGAACTGGGCAATACCCCTACCTCT
GATGAGCTATGGGCCTTGGCAGTGCAGGAGAGGAGGCGTCCCTACATCCC
ATCCACCTGGCGCTGCTTTGCCACAGACCCTGATGGGCTGAGGGAGCTCC
TAGAAGACTGTTGGGATGCAGACCCAGAAGCACGGCTGACAGCTGAGTGT
GTACAGCAGCGCCTGGCTGCCTTGGCCCATCCTCAAGAGAGCCACCCCTT
TCCAGAGAGCTGTCCACGTGGCTGCCCACCTCTCTGCCCAGAAGACTGTA
CTTCAATTCCTGCCCCTACCATCCTCCCCTGTAGGCCTCAGCGGAGTGCC
TGCCACTTCAGCGTTCAGCAAGGCCCTTGTTCCAGGAATCCTCAGCCTGC
CTGTACCCTTTCTCCTGTG

A nucleic acid sequence encoding an extracellular human MISRII polypeptide is as follows:

(SEQ ID NO: 53)

CCCCCAAACAGGCGAACCTGTGTGTTCTTTGAGGCCCCTGGAGTGCGGGG
AAGCACAAAGACACTGGGAGAGCTGCTAGATACAGGCACAGAGCTCCCCA
GAGCTATCCGCTGCCTCTACAGCCGCTGCTGCTTTGGGATCTGGAACCTG
ACCCAAGACCGGGCACAGGTGGAAATGCAAGGATGCCGAGACAGTGATGA
GCCAGGCTGTGAGTCCCTCCACTGTGACCCAAGTCCCCGAGCCCACCCCA
GCCCTGGCTCCACTCTCTTCACCTGCTCCTGTGGCACTGACTTCTGCAAT
GCCAATTACAGCCATCTGCCTCCTCCAGGGAGCCCTGGGACTCCTGGCTC
CCAGGGTCCCCAGGCTGCCCCAGGTGAGTCCATCTGGATGGCACTG

An alternative isoform of the human MISRII precursor protein sequence, isoform 2 (NCBI Ref Seq NP_001158162.1), is as follows:

| (SEQ ID NO: 75) | |
|---|---|
| 1 | MLGSLGLWAL LPTAVEAPPN RRTCVFFEAP GVRGSTKTLG ELLDTGTELP |
| 051 | RAIRCLYSRC CFGIWNLTQD RAQVEMQGCR DSDEPGCESL HCDPSPRAHP |
| 101 | SPGSTLFTCS CGTDFCNANY SHLPPPGSPG TPGSQGPQAA PGESIWMALV |
| 151 | LLGLFLLLLL LLGSIILALL QRKNYRVRGE PVPEPRPDSG RDWSVELQEL |
| 201 | PELCFSQVIR EGGHAVVWAG QLQGKLVAIK AFPPRSVAQF QAERALYELP |
| 251 | GLQHDHIVRF ITASRGGPGR LLSGPLLVLE LHPKGSLCHY LTQYTSDWGS |
| 301 | SLRMALSLAQ GLAFLHEERW QNGQYKPGIA HRDLSSQNVL IREDGSCAIG |
| 351 | DLGLALVLPG LTQPPAWTPT QPQGPAAIME AGTQRYMAPE LLDKTLDLQD |
| 401 | WGMALRRADI YSLALLLWEI LSRCPDLRPA VHHPSNWPMR QNWAIPLPLM |
| 451 | SYGPWQCRRG GVPTSHPPGA ALPQTLMG |

The signal peptide is indicated by a single underline and the extracellular domain is indicated in bold font.

A processed extracellular MISRII polypeptide sequence (isoform 2) is as follows:

(SEQ ID NO: 76)

PPNRRTCVFFEAPGVRGSTKTLGELLDTGTELPRAIRCLYSRCCFGIWNL
TQDRAQVEMQGCRDSDEPGCESLHCDPSPRAHPSPGSTLFTCSCGTDFCN
ANYSHLPPPGSPGTPGSQGPQAAPGESIWMAL

A nucleic acid sequence encoding the MISRII precursor protein (isoform 2) is shown below (SEQ ID NO: 77), corresponding to nucleotides 81-1514 of Genbank Reference Sequence NM_001164690.1. The signal sequence is underlined.

(SEQ ID NO: 77)

ATGCTAGGGTCTTTGGGGCTTTGGGCATTACTTCCCACAGCTGTGGAAGC
ACCCCCAAACAGGCGAACCTGTGTGTTCTTTGAGGCCCCTGGAGTGCGGG
GAAGCACAAAGACACTGGGAGAGCTGCTAGATACAGGCACAGAGCTCCCC
AGAGCTATCCGCTGCCTCTACAGCCGCTGCTGCTTTGGGATCTGGAACCT
GACCCAAGACCGGGCACAGGTGGAAATGCAAGGATGCCGAGACAGTGATG
AGCCAGGCTGTGAGTCCCTCCACTGTGACCCAAGTCCCCGAGCCCACCCC
AGCCCTGGCTCCACTCTCTTCACCTGCTCCTGTGGCACTGACTTCTGCAA
TGCCAATTACAGCCATCTGCCTCCTCCAGGGAGCCCTGGGACTCCTGGCT
CCCAGGGTCCCCAGGCTGCCCCAGGTGAGTCCATCTGGATGGCACTGGTG
CTGCTGGGGCTGTTCCTCCTCCTCCTGCTGCTGCTGGGCAGCATCATCTT
GGCCCTGCTACAGCGAAAGAACTACAGAGTGCGAGGTGAGCCAGTGCCAG
AGCCAAGGCCAGACTCAGGCAGGGACTGGAGTGTGGAGCTGCAGGAGCTG
CCTGAGCTGTGTTTCTCCCAGGTAATCCGGGAAGGAGGTCATGCAGTGGT
TTGGGCCGGGCAGCTGCAAGGAAAACTGGTTGCCATCAAGGCCTTCCCAC
CGAGGTCTGTGGCTCAGTTCCAAGCTGAGAGAGCATTGTACGAACTTCCA
GGCCTACAGCACGACCACATTGTCCGATTTATCACTGCCAGCCGGGGGGG
TCCTGGCCGCCTGCTCTCTGGGCCCCTGCTGGTACTGGAACTGCATCCCA
AGGGCTCCCTGTGCCACTACTTGACCCAGTACACCAGTGACTGGGGAAGT
TCCCTGCGGATGGCACTGTCCCTGGCCCAGGGCCTGGCATTTCTCCATGA
GGAGCGCTGGCAGAATGGCCAATATAAACCAGGTATTGCCCACCGAGATC
TGAGCAGCCAGAATGTGCTCATTCGGGAAGATGGATCGTGTGCCATTGGA
GACCTGGGCCTTGCCTTGGTGCTCCCTGGCCTCACTCAGCCCCCTGCCTG
GACCCCTACTCAACCACAAGGCCCAGCTGCCATCATGGAAGCTGGCACCC
AGAGGTACATGGCACCAGAGCTCTTGGACAAGACTCTGGACCTACAGGAT
TGGGGCATGGCCCTCCGACGAGCTGATATTTACTCTTTGGCTCTGCTCCT
GTGGGAGATACTGAGCCGCTGCCCAGATTTGAGGCCTGCAGTCCACCACC
CTTCCAACTGGCCTATGAGGCAGAACTGGGCAATACCCCTACCTCTGATG
AGCTATGGGCCTTGGCAGTGCAGGAGAGGAGGCGTCCCTACATCCCATCC
ACCTGGCGCTGCTTTGCCACAGACCCTGATGGGC

The nucleic acid sequence encoding a processed soluble (extracellular) human MISRII polypeptide (isoform 2) is as follows:

(SEQ ID NO: 78)

CCCCCAAACAGGCGAACCTGTGTGTTCTTTGAGGCCCCTGGAGTGCGGGG
AAGCACAAAGACACTGGGAGAGCTGCTAGATACAGGCACAGAGCTCCCCA
GAGCTATCCGCTGCCTCTACAGCCGCTGCTGCTTTGGGATCTGGAACCTG
ACCCAAGACCGGGCACAGGTGGAAATGCAAGGATGCCGAGACAGTGATGA
GCCAGGCTGTGAGTCCCTCCACTGTGACCCAAGTCCCCGAGCCCACCCCA
GCCCTGGCTCCACTCTCTTCACCTGCTCCTGTGGCACTGACTTCTGCAAT
GCCAATTACAGCCATCTGCCTCCTCCAGGGAGCCCTGGGACTCCTGGCTC
CCAGGGTCCCCAGGCTGCCCCAGGTGAGTCCATCTGGATGGCACTG

An alternative isoform of the human MISRII precursor protein sequence, isoform 3 (NCBI Ref Seq NP_001158163.1), is as follows:

| (SEQ ID NO: 79) | |
|---|---|
| 1 | MLGSLGLWAL LPTAVEAPPN RRTCVFFEAP GVRGSTKTLG ELLDTGTELP |
| 51 | RAIRCLYSRC CFGIWNLTQD RAQVEMQGCR DSDEPGCESL HCDPSPRAHP |
| 101 | SPGSTLFTCS CGTDFCNANY SHLPPPGSPG TPGSQGPQAA PGESIWMALV |
| 151 | LLGLFLLLLL LLGSIILALL QRKNYRVRGE PVPEPRPDSG RDWSVELQEL |
| 201 | PELCFSQVIR EGGHAVVWAG QLQGKLVAIK AFPPRSVAQF QAERALYELP |
| 251 | GLQHDHIVRF ITASRGGPGR LLSGPLLVLE LHPKGSLCHY LTQYTSDWGS |
| 301 | SLRMALSLAQ GLAFLHEERW QNGQYKPGIA HRDLSSQNVL IREDGSCAIG |
| 351 | DLGLALVLPG LTQPPAWTPT QPQGPAAIME DPDGLRELLE DCWDADPEAR |
| 401 | LTAECVQQRL AALAHPQESH PFPESCPRGC PPLCPEDCTS IPAPTILPCR |
| 451 | PQRSACHFSV QQGPCSRNPQ PACTLSPV |

The signal peptide is indicated by a single underline and the extracellular domain is indicated in bold font.

A processed extracellular MISRII polypeptide sequence (isoform 3) is as follows:

(SEQ ID NO: 80)

PPNRRTCVFFEAPGVRGSTKTLGELLDTGTELPRAIRCLYSRCCFGIWNL
TQDRAQVEMQGCRDSDEPGCESLHCDPSPRAHPSPGSTLFTCSCGTDFCN
ANYSHLPPPGSPGTPGSQGPQAAPGESIWMAL

A nucleic acid sequence encoding human MISRII precursor protein (isoform 3) is shown below (SEQ ID NO: 81), corresponding to nucleotides 81-1514 of Genbank Reference Sequence NM_001164691.1. The signal sequence is underlined.

(SEQ ID NO: 81)

ATGCTAGGGTCTTTGGGGCTTTGGGCATTACTTCCCACAGCTGTGGAAGC
ACCCCCAAACAGGCGAACCTGTGTGTTCTTTGAGGCCCCTGGAGTGCGGG
GAAGCACAAAGACACTGGGAGAGCTGCTAGATACAGGCACAGAGCTCCCC
AGAGCTATCCGCTGCCTCTACAGCCGCTGCTGCTTTGGGATCTGGAACCT
GACCCAAGACCGGGCACAGGTGGAAATGCAAGGATGCCGAGACAGTGATG
AGCCAGGCTGTGAGTCCCTCCACTGTGACCCAAGTCCCCGAGCCCACCCC
AGCCCTGGCTCCACTCTCTTCACCTGCTCCTGTGGCACTGACTTCTGCAA
TGCCAATTACAGCCATCTGCCTCCTCCAGGGAGCCCTGGGACTCCTGGCT
CCCAGGGTCCCCAGGCTGCCCCAGGTGAGTCCATCTGGATGGCACTGGTG
CTGCTGGGGCTGTTCCTCCTCCTCCTGCTGCTGCTGGGCAGCATCATCTT
GGCCCTGCTACAGCGAAAGAACTACAGAGTGCGAGGTGAGCCAGTGCCAG
AGCCAAGGCCAGACTCAGGCAGGGACTGGAGTGTGGAGCTGCAGGAGCTG
CCTGAGCTGTGTTTCTCCCAGGTAATCCGGGAAGGAGGTCATGCAGTGGT
TTGGGCCGGGCAGCTGCAAGGAAAACTGGTTGCCATCAAGGCCTTCCCAC
CGAGGTCTGTGGCTCAGTTCCAAGCTGAGAGAGCATTGTACGAACTTCCA
GGCCTACAGCACGACCACATTGTCCGATTTATCACTGCCAGCCGGGGGGG
TCCTGGCCGCCTGCTCTCTGGGCCCCTGCTGGTACTGGAACTGCATCCCA
AGGGCTCCCTGTGCCACTACTTGACCCAGTACACCAGTGACTGGGGAAGT
TCCCTGCGGATGGCACTGTCCCTGGCCCAGGGCCTGGCATTTCTCCATGA
GGAGCGCTGGCAGAATGGCCAATATAAACCAGGTATTGCCCACCGAGATC
TGAGCAGCCAGAATGTGCTCATTCGGGAAGATGGATCGTGTGCCATTGGA
GACCTGGGCCTTGCCTTGGTGCTCCCTGGCCTCACTCAGCCCCCTGCCTG
GACCCCTACTCAACCACAAGGCCCAGCTGCCATCATGGAAGACCCTGATG
GGCTGAGGGAGCTCCTAGAAGACTGTTGGGATGCAGACCCAGAAGCACGG
CTGACAGCTGAGTGTGTACAGCAGCGCCTGGCTGCCTTGGCCCATCCTCA
AGAGAGCCACCCCTTTCCAGAGAGCTGTCCACGTGGCTGCCCACCTCTCT
GCCCAGAAGACTGTACTTCAATTCCTGCCCCTACCATCCTCCCCTGTAGG
CCTCAGCGGAGTGCCTGCCACTTCAGCGTTCAGCAAGGCCCTTGTTCCAG
GAATCCTCAGCCTGCCTGTACCCTTTCTCCTGTG

A nucleic acid sequence encoding a processed soluble (extracellular) human MISRII polypeptide (isoform 3) is as follows:

(SEQ ID NO: 82)

CCCCCAAACAGGCGAACCTGTGTGTTCTTTGAGGCCCCTGGAGTGCGGGG
AAGCACAAAGACACTGGGAGAGCTGCTAGATACAGGCACAGAGCTCCCCA
GAGCTATCCGCTGCCTCTACAGCCGCTGCTGCTTTGGGATCTGGAACCTG
ACCCAAGACCGGGCACAGGTGGAAATGCAAGGATGCCGAGACAGTGATGA
GCCAGGCTGTGAGTCCCTCCACTGTGACCCAAGTCCCCGAGCCCACCCCA
GCCCTGGCTCCACTCTCTTCACCTGCTCCTGTGGCACTGACTTCTGCAAT
GCCAATTACAGCCATCTGCCTCCTCCAGGGAGCCCTGGGACTCCTGGCTC
CCAGGGTCCCCAGGCTGCCCCAGGTGAGTCCATCTGGATGGCACTG

In certain embodiments, the disclosure relates to heteromultimers that comprise at least one MISRII polypeptide, which includes fragments, functional variants, and modified forms thereof. Preferably, MISRII polypeptides for use in accordance with the disclosure (e.g., heteromultimers comprising a MISRII polypeptide and uses thereof) are soluble (e.g., an extracellular domain of MISRII). In other preferred embodiments, MISRII polypeptides for use in accordance with the disclosure bind to and/or inhibit (antagonize) activity (e.g., Smad signaling) of one or more TGF-beta superfamily ligands. In some embodiments, heteromultimers of the disclosure comprise at least one MISRII polypeptide that is at least 70%, 75%, 80%, 85%, 90%, 91%, 92%, 93%, 94%, 95%, 96%, 97%, 98%, 99%, or 100% identical to the amino acid sequence of SEQ ID NOs: 50, 51, 75, 76, 79, or 80. In some embodiments, heteromultimers of the disclosure comprise at least one MISRII polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 18-24 (e.g., amino acid residues 18, 19, 20, 21, 22, 23, or 24) of SEQ ID NO: 50, and ends at any one of amino acids 116-149 (e.g., amino acid residues 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, or 149) of SEQ ID NO: 50. In some embodiments, heteromultimers of the disclosure comprise at least one MISRII polypeptide that is at least 70%, 75%, 80%, 85%, 90%, 91%, 92%, 93%, 94%, 95%, 96%, 97%, 98%, 99%, or 100% identical to amino acids of 18-116 of SEQ ID NO: 50. In some embodiments, heteromultimers of the disclosure comprise at least one MISRII polypeptide that is at least 70%, 75%, 80%, 85%, 90%, 91%, 92%, 93%, 94%, 95%, 96%, 97%, 98%, 99%, or 100% identical to amino acids of 18-149 of SEQ ID NO: 50. In some embodiments, heteromultimers of the disclosure comprise at least one MISRII polypeptide that is at least 70%, 75%, 80%, 85%, 90%, 91%, 92%, 93%, 94%, 95%, 96%, 97%, 98%, 99%, or 100% identical to amino acids of 24-116 of SEQ ID NO: 50. In some embodiments, heteromultimers of the disclosure comprise at least one MISRII polypeptide that is at least 70%, 75%, 80%, 85%, 90%, 91%, 92%, 93%, 94%, 95%, 96%, 97%, 98%, 99%, or 100% identical to amino acids of 24-149 of SEQ ID NO: 50. In some embodiments, heteromultimers of the disclosure comprise at least one MISRII polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 18-24 (e.g., amino acid residues 18, 19, 20, 21, 22, 23, or 24) of SEQ ID NO: 75, and ends at any one of amino acids 116-149 (e.g., amino acid residues 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, or 149) of SEQ ID NO: 75. In some embodiments, heteromultimers of the disclosure comprise at least one MISRII polypeptide that is at least 70%, 75%, 80%, 85%, 90%, 91%, 92%, 93%, 94%, 95%, 96%, 97%, 98%, 99%, or 100% identical to amino acids of 18-116 of SEQ ID NO: 75. In some embodiments, heteromultimers of the disclosure comprise at least one MISRII polypeptide that is at least 70%, 75%, 80%, 85%, 90%, 91%, 92%, 93%, 94%, 95%, 96%, 97%, 98%, 99%, or 100% identical to amino acids of 18-149 of SEQ ID NO: 75. In some embodiments, heteromultimers of the disclosure comprise at least one MISRII polypeptide that is at least 70%, 75%, 80%, 85%, 90%, 91%, 92%, 93%, 94%, 95%, 96%, 97%, 98%, 99%, or 100% identical to amino acids of 24-116 of SEQ ID NO: 75. In some embodiments, heteromultimers of the disclosure comprise at least one MISRII polypeptide that is at least 70%, 75%, 80%, 85%, 90%, 91%, 92%, 93%, 94%, 95%, 96%, 97%, 98%, 99%, or 100% identical to amino acids of 24-149 of SEQ ID NO: 75. In some embodiments, heteromultimers of the disclosure comprise at least one MISRII polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 18-24 (e.g., amino acid residues 18, 19, 20, 21, 22, 23, or 24) of SEQ ID NO: 50, and ends at any one of amino acids 116-149 (e.g., amino acid residues 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, or 149) of SEQ ID NO: 79. In some embodiments, heteromultimers of the disclosure comprise at least one MISRII polypeptide that is at least 70%, 75%, 80%, 85%, 90%, 91%, 92%, 93%, 94%, 95%, 96%, 97%, 98%, 99%, or 100% identical to amino acids of 18-116 of SEQ ID NO: 79. In some embodiments, heteromultimers of the disclosure comprise at least one MISRII polypeptide that is at least 70%, 75%, 80%, 85%, 90%, 91%, 92%, 93%, 94%, 95%, 96%, 97%, 98%, 99%, or 100% identical to amino acids of 18-149 of SEQ ID NO: 79. In some embodiments, heteromultimers of the disclosure comprise at least one MISRII polypeptide that is at least 70%, 75%, 80%, 85%, 90%, 91%, 92%, 93%, 94%, 95%, 96%, 97%, 98%, 99%, or 100% identical to amino acids of 24-116 of SEQ ID NO: 79. In some embodiments, heteromultimers of the disclosure comprise at least one MISRII polypeptide that is at least 70%, 75%, 80%, 85%, 90%, 91%, 92%, 93%, 94%, 95%, 96%, 97%, 98%, 99%, or 100% identical to amino acids of 24-149 of SEQ ID NO: 79.

In certain aspects, the present disclosure relates to heteromultimers that comprise an ALK1 polypeptide. As used herein, the term “ALK1” refers to a family of activin receptor-like kinase-1 proteins from any species and variants derived from such ALK1 proteins by mutagenesis or other modification. Reference to ALK1 herein is understood to be a reference to any one of the currently identified forms. Members of the ALK1 family are generally transmembrane proteins, composed of a ligand-binding extracellular domain with a cysteine-rich region, a transmembrane domain, and a cytoplasmic domain with predicted serine/threonine kinase activity.

The term “ALK1 polypeptide” includes polypeptides comprising any naturally occurring polypeptide of an ALK1 family member as well as any variants thereof (including mutants, fragments, fusions, and peptidomimetic forms) that retain a useful activity.

A human ALK1 precursor protein sequence (NCBI Ref Seq NP_000011.2) is as follows:

| (SEQ ID NO: 14) | |
|---|---|
| 1 | MTLGSPRKGL LMLLMALVTQ GDPVKPSRGP LVTCTCESPH CKGPTCRGAW |
| 51 | CTVVLVREEG RHPQEHRGCG NLHRELCRGR PTEFVNHYCC DSHLCNHNVS |
| 101 | LVLEATQPPS EQPGTDGQLA LILGPVLALL ALVALGVLGL WHVRRRQEKQ |
| 151 | RGLHSELGES SLILKASEQG DSMLGDLLDS DCTTGSGSGL PFLVQRTVAR |
| 201 | QVALVECVGK GRYGEVWRGL WHGESVAVKI FSSRDEQSWF RETEIYNTVL |
| 251 | LRHDNILGFI ASDMTSRNSS TQLWLITHYH EHGSLYDFLQ RQTLEPHLAL |
| 301 | RLAVSAACGL AHLHVEIFGT QGKPAIAHRD FKSRNVLVKS NLQCCIADLG |
| 351 | LAVMHSQGSD YLDIGNNPRV GTKRYMAPEV LDEQIRTDCF ESYKWTDIWA |
| 401 | FGLVLWEIAR RTIVNGIVED YRPPFYDVVP NDPSFEDMKK VVCVDQQTPT |
| 451 | IPNRLAADPV LSGLAQMMRE CWYPNPSARL TALRIKKTLQ KISNSPEKPK |
| 501 | VIQ |

The signal peptide is indicated by a single underline and the extracellular domain is indicated in bold font.

A processed extracellular ALK1 polypeptide sequence is as follows:

(SEQ ID NO: 15)

DPVKPSRGPLVTCTCESPHCKGPTCRGAWCTVVLVREEGRHPQEHRGCGN
LHRELCRGRPTEFVNHYCCDSHLCNHNVSLVLEATQPPSEQPGTDGQ

A nucleic acid sequence encoding human ALK1 precursor protein is shown below (SEQ ID NO: 16), corresponding to nucleotides 284-1792 of Genbank Reference Sequence NM_000020.2. The signal sequence is underlined.

(SEQ ID NO: 16)

ATGACCTTGGGCTCCCCCAGGAAAGGCCTTCTGATGCTGCTGATGGCCTT
GGTGACCCAGGGAGACCCTGTGAAGCCGTCTCGGGGCCCGCTGGTGACCT
GCACGTGTGAGAGCCCACATTGCAAGGGGCCTACCTGCCGGGGGGCCTGG
TGCACAGTAGTGCTGGTGCGGGAGGAGGGGAGGCACCCCCAGGAACATCG
GGGCTGCGGGAACTTGCACAGGGAGCTCTGCAGGGGGCGCCCCACCGAGT
TCGTCAACCACTACTGCTGCGACAGCCACCTCTGCAACCACAACGTGTCC
CTGGTGCTGGAGGCCACCCAACCTCCTTCGGAGCAGCCGGGAACAGATGG
CCAGCTGGCCCTGATCCTGGGCCCCGTGCTGGCCTTGCTGGCCCTGGTGG
CCCTGGGTGTCCTGGGCCTGTGGCATGTCCGACGGAGGCAGGAGAAGCAG
CGTGGCCTGCACAGCGAGCTGGGAGAGTCCAGTCTCATCCTGAAAGCATC
TGAGCAGGGCGACAGCATGTTGGGGGACCTCCTGGACAGTGACTGCACCA
CAGGGAGTGGCTCAGGGCTCCCCTTCCTGGTGCAGAGGACAGTGGCACGG
CAGGTTGCCTTGGTGGAGTGTGTGGGAAAAGGCCGCTATGGCGAAGTGTG
GCGGGGCTTGTGGCACGGTGAGAGTGTGGCCGTCAAGATCTTCTCCTCGA
GGGATGAACAGTCCTGGTTCCGGGAGACTGAGATCTATAACACAGTGTTG
CTCAGACACGACAACATCCTAGGCTTCATCGCCTCAGACATGACCTCCCG
CAACTCGAGCACGCAGCTGTGGCTCATCACGCACTACCACGAGCACGGCT
CCCTCTACGACTTTCTGCAGAGACAGACGCTGGAGCCCCATCTGGCTCTG
AGGCTAGCTGTGTCCGCGGCATGCGGCCTGGCGCACCTGCACGTGGAGAT
CTTCGGTACACAGGGCAAACCAGCCATTGCCCACCGCGACTTCAAGAGCC
GCAATGTGCTGGTCAAGAGCAACCTGCAGTGTTGCATCGCCGACCTGGGC
CTGGCTGTGATGCACTCACAGGGCAGCGATTACCTGGACATCGGCAACAA
CCCGAGAGTGGGCACCAAGCGGTACATGGCACCCGAGGTGCTGGACGAGC
AGATCCGCACGGACTGCTTTGAGTCCTACAAGTGGACTGACATCTGGGCC
TTTGGCCTGGTGCTGTGGGAGATTGCCCGCCGGACCATCGTGAATGGCAT
CGTGGAGGACTATAGACCACCCTTCTATGATGTGGTGCCCAATGACCCCA
GCTTTGAGGACATGAAGAAGGTGGTGTGTGTGGATCAGCAGACCCCCACC
ATCCCTAACCGGCTGGCTGCAGACCCGGTCCTCTCAGGCCTAGCTCAGAT
GATGCGGGAGTGCTGGTACCCAAACCCCTCTGCCCGACTCACCGCGCTGC
GGATCAAGAAGACACTACAAAAAATTAGCAACAGTCCAGAGAAGCCTAAA
GTGATTCAA

A nucleic acid sequence encoding a processed extracellular ALK1 polypeptide is as follows:

(SEQ ID NO: 17)

GACCCTGTGAAGCCGTCTCGGGGCCCGCTGGTGACCTGCACGTGTGAGAG
CCCACATTGCAAGGGGCCTACCTGCCGGGGGGCCTGGTGCACAGTAGTGC
TGGTGCGGGAGGAGGGGAGGCACCCCCAGGAACATCGGGGCTGCGGGAAC
TTGCACAGGGAGCTCTGCAGGGGGCGCCCCACCGAGTTCGTCAACCACTA
CTGCTGCGACAGCCACCTCTGCAACCACAACGTGTCCCTGGTGCTGGAGG
CCACCCAACCTCCTTCGGAGCAGCCGGGAACAGATGGCCAG

In certain embodiments, the disclosure relates to heteromultimers that comprise at least one ALK1 polypeptide, which includes fragments, functional variants, and modified forms thereof. Preferably, ALK1 polypeptides for use in accordance with the disclosure (e.g., heteromultimers comprising an ALK1 polypeptide and uses thereof) are soluble (e.g., an extracellular domain of ALK1). In other preferred embodiments, ALK1 polypeptides for use in accordance with the disclosure bind to and/or inhibit (antagonize) activity (e.g., Smad signaling) of one or more TGF-beta superfamily ligands. In some embodiments, heteromultimers of the disclosure comprise at least one ALK1 polypeptide that is at least 70%, 75%, 80%, 85%, 90%, 91%, 92%, 93%, 94%, 95%, 96%, 97%, 98%, 99%, or 100% identical to the amino acid sequence of SEQ ID NOs: 14 or 15. In some embodiments, heteromultimers of the disclosure comprise at least one ALK1 polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 22-34 (e.g., amino acid residues 22, 23, 24, 25, 26, 27, 28, 29, 30, 31, 32, 33, or 34) of SEQ ID NO: 14, and ends at any one of amino acids 95-118 (e.g., amino acid residues 95, 96, 97, 98, 99, 100, 101, 102, 103, 104, 105, 106, 107, 108, 109, 110, 111, 112, 113, 114, 115, 116, 117, or 118) of SEQ ID NO: 14. In some embodiments, heteromultimers of the disclosure comprise at least one ALK1 polypeptide that is at least 70%, 75%, 80%, 85%, 90%, 91%, 92%, 93%, 94%, 95%, 96%, 97%, 98%, 99%, or 100% identical to amino acids of 22-95 of SEQ ID NO: 14. In some embodiments, heteromultimers of the disclosure comprise at least one ALK1 polypeptide that is at least 70%, 75%, 80%, 85%, 90%, 91%, 92%, 93%, 94%, 95%, 96%, 97%, 98%, 99%, or 100% identical to amino acids of 22-118 of SEQ ID NO: 14. In some embodiments, heteromultimers of the disclosure comprise at least one ALK1 polypeptide that is at least 70%, 75%, 80%, 85%, 90%, 91%, 92%, 93%, 94%, 95%, 96%, 97%, 98%, 99%, or 100% identical to amino acids of 34-95 of SEQ ID NO: 14. In some embodiments, heteromultimers of the disclosure comprise at least one ALK1 polypeptide that is at least 70%, 75%, 80%, 85%, 90%, 91%, 92%, 93%, 94%, 95%, 96%, 97%, 98%, 99%, or 100% identical to amino acids of 34-118 of SEQ ID NO: 14.

In certain aspects, the present disclosure relates to heteromultimers that comprise an ALK2 polypeptide. As used herein, the term “ALK2” refers to a family of activin receptor-like kinase-2 proteins from any species and variants derived from such ALK2 proteins by mutagenesis or other modification. Reference to ALK2 herein is understood to be a reference to any one of the currently identified forms. Members of the ALK2 family are generally transmembrane proteins, composed of a ligand-binding extracellular domain with a cysteine-rich region, a transmembrane domain, and a cytoplasmic domain with predicted serine/threonine kinase activity.

The term “ALK2 polypeptide” includes polypeptides comprising any naturally occurring polypeptide of an ALK2 family member as well as any variants thereof (including mutants, fragments, fusions, and peptidomimetic forms) that retain a useful activity.

A human ALK2 precursor protein sequence (NCBI Ref Seq NP_001096.1) is as follows:

| (SEQ ID NO: 18) | |
|---|---|
| 1 | MVDGVMILPV LIMIALPSPS MEDEKPKVNP KLYMCVCEGL SCGNEDHCEG |
| 51 | QQCFSSLSIN DGFHVYQKGC FQVYEQGKMT CKTPPSPGQA VECCQGDWCN |
| 101 | RNITAQLPTK GKSFPGTQNF HLEVGLIILS VVFAVCLLAC LLGVALRKFK |
| 151 | RRNQERLNPR DVEYGTIEGL ITTNVGDSTL ADLLDHSCTS GSGSGLPFLV |
| 201 | QRTVARQITL LECVGKGRYG EVWRGSWQGE NVAVKIFSSR DEKSWFRETE |
| 251 | LYNTVMLRHE NILGFIASDM TSRHSSTQLW LITHYHEMGS LYDYLQLTTL |
| 301 | DTVSCLRIVL SIASGLAHLH IEIFGTQGKP AIAHRDLKSK NILVKKNGQC |
| 351 | CIADLGLAVM HSQSTNQLDV GNNPRVGTKR YMAPEVLDET IQVDCFDSYK |
| 401 | RVDIWAFGLV LWEVARRMVS NGIVEDYKPP FYDVVPNDPS FEDMRKVVCV |
| 451 | DQQRPNIPNR WFSDPTLTSL AKLMKECWYQ NPSARLTALR IKKTLTKIDN |
| 501 | SLDKLKTDC |

The signal peptide is indicated by a single underline and the extracellular domain is indicated in bold font.

A processed extracellular ALK2 polypeptide sequence is as follows:

(SEQ ID NO: 19)

MEDEKPKVNPKLYMCVCEGLSCGNEDHCEGQQCFSSLSINDGFHVYQKGC
FQVYEQGKMTCKTPPSPGQAVECCQGDWCNRNITAQLPTKGKSFPGTQNF
HLE

A nucleic acid sequence encoding human ALK2 precursor protein is shown below (SEQ ID NO: 20), corresponding to nucleotides 431-1957 of Genbank Reference Sequence NM_001105.4. The signal sequence is underlined.

(SEQ ID NO: 20)

ATGGTAGATGGAGTGATGATTCTTCCTGTGCTTATCATGATTGCTCTCCC
CTCCCCTAGTATGGAAGATGAGAAGCCCAAGGTCAACCCCAAACTCTACA
TGTGTGTGTGTGAAGGTCTCTCCTGCGGTAATGAGGACCACTGTGAAGGC
CAGCAGTGCTTTTCCTCACTGAGCATCAACGATGGCTTCCACGTCTACCA
GAAAGGCTGCTTCCAGGTTTATGAGCAGGGAAAGATGACCTGTAAGACCC
CGCCGTCCCCTGGCCAAGCCGTGGAGTGCTGCCAAGGGGACTGGTGTAAC
AGGAACATCACGGCCCAGCTGCCCACTAAAGGAAAATCCTTCCCTGGAAC
ACAGAATTTCCACTTGGAGGTTGGCCTCATTATTCTCTCTGTAGTGTTCG
CAGTATGTCTTTTAGCCTGCCTGCTGGGAGTTGCTCTCCGAAAATTTAAA
AGGCGCAACCAAGAACGCCTCAATCCCCGAGACGTGGAGTATGGCACTAT
CGAAGGGCTCATCACCACCAATGTTGGAGACAGCACTTTAGCAGATTTAT
TGGATCATTCGTGTACATCAGGAAGTGGCTCTGGTCTTCCTTTTCTGGTA
CAAAGAACAGTGGCTCGCCAGATTACACTGTTGGAGTGTGTCGGGAAAGG
CAGGTATGGTGAGGTGTGGAGGGGCAGCTGGCAAGGGGAGAATGTTGCCG
TGAAGATCTTCTCCTCCCGTGATGAGAAGTCATGGTTCAGGGAAACGGAA
TTGTACAACACTGTGATGCTGAGGCATGAAAATATCTTAGGTTTCATTGC
TTCAGACATGACATCAAGACACTCCAGTACCCAGCTGTGGTTAATTACAC
ATTATCATGAAATGGGATCGTTGTACGACTATCTTCAGCTTACTACTCTG
GATACAGTTAGCTGCCTTCGAATAGTGCTGTCCATAGCTAGTGGTCTTGC
ACATTTGCACATAGAGATATTTGGGACCCAAGGGAAACCAGCCATTGCCC
ATCGAGATTTAAAGAGCAAAAATATTCTGGTTAAGAAGAATGGACAGTGT
TGCATAGCAGATTTGGGCCTGGCAGTCATGCATTCCCAGAGCACCAATCA
GCTTGATGTGGGGAACAATCCCCGTGTGGGCACCAAGCGCTACATGGCCC
CCGAAGTTCTAGATGAAACCATCCAGGTGGATTGTTTCGATTCTTATAAA
AGGGTCGATATTTGGGCCTTTGGACTTGTTTTGTGGGAAGTGGCCAGGCG
GATGGTGAGCAATGGTATAGTGGAGGATTACAAGCCACCGTTCTACGATG
TGGTTCCCAATGACCCAAGTTTTGAAGATATGAGGAAGGTAGTCTGTGTG
GATCAACAAAGGCCAAACATACCCAACAGATGGTTCTCAGACCCGACATT
AACCTCTCTGGCCAAGCTAATGAAAGAATGCTGGTATCAAAATCCATCCG
CAAGACTCACAGCACTGCGTATCAAAAAGACTTTGACCAAAATTGATAAT
TCCCTCGACAAATTGAAAACTGACTGT

A nucleic acid sequence encoding an extracellular ALK2 polypeptide is as follows:

(SEQ ID NO: 21)

ATGGAAGATGAGAAGCCCAAGGTCAACCCCAAACTCTACATGTGTGTGTG
TGAAGGTCTCTCCTGCGGTAATGAGGACCACTGTGAAGGCCAGCAGTGCT
TTTCCTCACTGAGCATCAACGATGGCTTCCACGTCTACCAGAAAGGCTGC
TTCCAGGTTTATGAGCAGGGAAAGATGACCTGTAAGACCCCGCCGTCCCC
TGGCCAAGCCGTGGAGTGCTGCCAAGGGGACTGGTGTAACAGGAACATCA
CGGCCCAGCTGCCCACTAAAGGAAAATCCTTCCCTGGAACACAGAATTTC
CACTTGGAG

In certain embodiments, the disclosure relates to heteromultimers that comprise at least one ALK2 polypeptide, which includes fragments, functional variants, and modified forms thereof. Preferably, ALK2 polypeptides for use in accordance with the disclosure (e.g., heteromultimers comprising an ALK2 polypeptide and uses thereof) are soluble (e.g., an extracellular domain of ALK2). In other preferred embodiments, ALK2 polypeptides for use in accordance with the disclosure bind to and/or inhibit (antagonize) activity (e.g., Smad signaling) of one or more TGF-beta superfamily ligands. In some embodiments, heteromultimers of the disclosure comprise at least one ALK2 polypeptide that is at least 70%, 75%, 80%, 85%, 90%, 91%, 92%, 93%, 94%, 95%, 96%, 97%, 98%, 99%, or 100% identical to the amino acid sequence of SEQ ID NO: 18 or 19. In some embodiments, heteromultimers of the disclosure consist or consist essentially of at least one ALK2 polypeptide that is at least 70%, 75%, 80%, 85%, 90%, 95%, 97%, 98%, or 99% identical to the amino acid sequence of SEQ ID NO: 18 or 19. In some embodiments, heteromultimers of the disclosure comprise at least one ALK2 polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 21-35 (e.g., amino acid residues 21, 22, 23, 24, 25, 26, 27, 28, 29, 30, 31, 32, 33, 34, or 35) of SEQ ID NO: 18, and ends at any one of amino acids 99-123 (e.g., amino acid residues 99, 100, 101, 102, 103, 104, 105, 106, 107, 108, 109, 110, 111, 112, 113, 114, 115, 116, 117, 118, 119, 120, 121, 122, or 123) of SEQ ID NO: 18. In some embodiments, heteromultimers of the disclosure comprise at least one ALK2 polypeptide that is at least 70%, 75%, 80%, 85%, 90%, 91%, 92%, 93%, 94%, 95%, 96%, 97%, 98%, 99%, or 100% identical to amino acids of 21-99 of SEQ ID NO: 18. In some embodiments, heteromultimers of the disclosure comprise at least one ALK2 polypeptide that is at least 70%, 75%, 80%, 85%, 90%, 91%, 92%, 93%, 94%, 95%, 96%, 97%, 98%, 99%, or 100% identical to amino acids of 21-123 of SEQ ID NO: 18. In some embodiments, heteromultimers of the disclosure comprise at least one ALK2 polypeptide that is at least 70%, 75%, 80%, 85%, 90%, 91%, 92%, 93%, 94%, 95%, 96%, 97%, 98%, 99%, or 100% identical to amino acids of 35-99 of SEQ ID NO: 18. In some embodiments, heteromultimers of the disclosure comprise at least one ALK2 polypeptide that is at least 70%, 75%, 80%, 85%, 90%, 91%, 92%, 93%, 94%, 95%, 96%, 97%, 98%, 99%, or 100% identical to amino acids of 35-123 of SEQ ID NO: 18.

In certain aspects, the present disclosure relates to heteromultimers that comprise an ALK3 polypeptide. As used herein, the term “ALK3” refers to a family of activin receptor-like kinase-3 proteins from any species and variants derived from such ALK3 proteins by mutagenesis or other modification. Reference to ALK3 herein is understood to be a reference to any one of the currently identified forms. Members of the ALK3 family are generally transmembrane proteins, composed of a ligand-binding extracellular domain with a cysteine-rich region, a transmembrane domain, and a cytoplasmic domain with predicted serine/threonine kinase activity.

The term “ALK3 polypeptide” includes polypeptides comprising any naturally occurring polypeptide of an ALK3 family member as well as any variants thereof (including mutants, fragments, fusions, and peptidomimetic forms) that retain a useful activity.

A human ALK3 precursor protein sequence (NCBI Ref Seq NP_004320.2) is as follows:

| (SEQ ID NO: 22) | |
|---|---|
| 1 | MPQLYIYIRL LGAYLFIISR VQGQNLDSML HGTGMKSDSD QKKSENGVTL APEDTLPFLK |
| 61 | CYCSGHCPDD AINNTCITNG HCFAIIEEDD QGETTLASGC MKYEGSDFQC KDSPKAQLRR |
| 121 | TIECCRTNLC NQYLQPTLPP VVIGPFFDGS IRWLVLLISM AVCIIAMIIF SSCFCYKHYC |
| 181 | KSISSRRRYN RDLEQDEAFI PVGESLKDLI DQSQSSGSGS GLPLLVQRTI AKQIQMVRQV |
| 241 | GKGRYGEVWM GKWRGEKVAV KVFFTTEEAS WFRETEIYQT VLMRHENILG FIAADIKGTG |
| 301 | SWTQLYLITD YHENGSLYDF LKCATLDTRA LLKLAYSAAC GLCHLHTEIY GTQGKPAIAH |
| 361 | RDLKSKNILI KKNGSCCIAD LGLAVKFNSD TNEVDVPLNT RVGTKRYMAP EVLDESLNKN |
| 421 | HFQPYIMADI YSFGLIIWEM ARRCITGGIV EEYQLPYYNM VPSDPSYEDM REVVCVKRLR |
| 481 | PIVSNRWNSD ECLRAVLKLM SECWAHNPAS RLTALRIKKT LAKMVESQDV KI |

The signal peptide is indicated by a single underline and the extracellular domain is indicated in bold font.

A processed extracellular ALK3 polypeptide sequence is as follows:

| (SEQ ID NO: 23) | |
|---|---|
| 1 | QNLDSMLHGT GMKSDSDQKK SENGVTLAPE DTLPFLKCYC SGHCPDDAIN NTCITNGHCF |
| 61 | AIIEEDDQGE TTLASGCMKY EGSDFQCKDS PKAQLRRTIE CCRTNLCNQY LQPTLPPVVI |
| 121 | GPFFDGSIR |

A nucleic acid sequence encoding human ALK3 precursor protein is shown below (SEQ ID NO: 24), corresponding to nucleotides 549-2144 of Genbank Reference Sequence NM_004329.2. The signal sequence is underlined and the extracellular domain is indicated in bold font.

| (SEQ ID NO: 24) | |
|---|---|
| 1 | ATGCCTCAGC TATACATTTA CATCAGATTA TTGGGAGCCT ATTTGTTCAT |
| | CATTTCTCGT |
| 61 | GTTCAAGGAC AGAATCTGGA TAGTATGCTT CATGGCACTG GGATGAAATC |
| | AGACTCCGAC |
| 121 | CAGAAAAAGT CAGAAAATGG AGTAACCTTA GCACCAGAGG ATACCTTGCC |
| | TTTTTTAAAG |
| 181 | TGCTATTGCT CAGGGCACTG TCCAGATGAT GCTATTAATA ACACATGCAT |
| | AACTAATGGA |
| 241 | CATTGCTTTG CCATCATAGA AGAAGATGAC CAGGGAGAAA CCACATTAGC |
| | TTCAGGGTGT |
| 301 | ATGAAATATG AAGGATCTGA TTTTCAGTGC AAAGATTCTC CAAAAGCCCA |
| | GCTACGCCGG |
| 361 | ACAATAGAAT GTTGTCGGAC CAATTTATGT AACCAGTATT TGCAACCCAC |
| | ACTGCCCCCT |
| 421 | GTTGTCATAG GTCCGTTTTT TGATGGCAGC ATTCGATGGC TGGTTTTGCT |
| | CATTTCTATG |
| 481 | GCTGTCTGCA TAATTGCTAT GATCATCTTC TCCAGCTGCT TTTGTTACAA |
| | ACATTATTGC |
| 541 | AAGAGCATCT CAAGCAGACG TCGTTACAAT CGTGATTTGG AACAGGATGA |
| | AGCATTTATT |
| 601 | CCAGTTGGAG AATCACTAAA AGACCTTATT GACCAGTCAC AAAGTTCTGG |
| | TAGTGGGTCT |
| 661 | GGACTACCTT TATTGGTTCA GCGAACTATT GCCAAACAGA TTCAGATGGT |
| | CCGGCAAGTT |
| 721 | GGTAAAGGCC GATATGGAGA AGTATGGATG GGCAAATGGC GTGGCGAAAA |
| | AGTGGCGGTG |
| 781 | AAAGTATTCT TTACCACTGA AGAAGCCAGC TGGTTTCGAG AAACAGAAAT |
| | CTACCAAACT |
| 841 | GTGCTAATGC GCCATGAAAA CATACTTGGT TTCATAGCGG CAGACATTAA |
| | AGGTACAGGT |
| 901 | TCCTGGACTC AGCTCTATTT GATTACTGAT TACCATGAAA ATGGATCTCT |
| | CTATGACTTC |
| 961 | CTGAAATGTG CTACACTGGA CACCAGAGCC CTGCTTAAAT TGGCTTATTC |
| | AGCTGCCTGT |
| 1021 | GGTCTGTGCC ACCTGCACAC AGAAATTTAT GGCACCCAAG GAAAGCCCGC |
| | AATTGCTCAT |
| 1081 | CGAGACCTAA AGAGCAAAAA CATCCTCATC AAGAAAAATG GGAGTTGCTG |
| | CATTGCTGAC |
| 1141 | CTGGGCCTTG CTGTTAAATT CAACAGTGAC ACAAATGAAG TTGATGTGCC |
| | CTTGAATACC |
| 1201 | AGGGTGGGCA CCAAACGCTA CATGGCTCCC GAAGTGCTGG ACGAAAGCCT |
| | GAACAAAAAC |
| 1261 | CACTTCCAGC CCTACATCAT GGCTGACATC TACAGCTTCG GCCTAATCAT |
| | TTGGGAGATG |
| 1321 | GCTCGTCGTT GTATCACAGG AGGGATCGTG GAAGAATACC AATTGCCATA |
| | TTACAACATG |
| 1381 | GTACCGAGTG ATCCGTCATA CGAAGATATG CGTGAGGTTG TGTGTGTCAA |
| | ACGTTTGCGG |
| 1441 | CCAATTGTGT CTAATCGGTG GAACAGTGAT GAATGTCTAC GAGCAGTTTT |
| | GAAGCTAATG |
| 1501 | TCAGAATGCT GGGCCCACAA TCCAGCCTCC AGACTCACAG CATTGAGAAT |
| | TAAGAAGACG |
| 1561 | CTTGCCAAGA TGGTTGAATC CCAAGATGTA AAAATC |

A nucleic acid sequence encoding an extracellular human ALK3 polypeptide is as follows:

| (SEQ ID NO: 25) | |
|---|---|
| 1 | CAGAATCTGG ATAGTATGCT TCATGGCACT GGGATGAAAT CAGACTCCGA |
| | CCAGAAAAAG |
| 61 | TCAGAAAATG GAGTAACCTT AGCACCAGAG GATACCTTGC CTTTTTTAAA |
| | GTGCTATTGC |
| 121 | TCAGGGCACT GTCCAGATGA TGCTATTAAT AACACATGCA TAACTAATGG |
| | ACATTGCTTT |
| 181 | GCCATCATAG AAGAAGATGA CCAGGGAGAA ACCACATTAG CTTCAGGGTG |
| | TATGAAATAT |
| 241 | GAAGGATCTG ATTTTCAGTG CAAAGATTCT CCAAAAGCCC AGCTACGCCG |
| | GACAATAGAA |
| 301 | TGTTGTCGGA CCAATTTATG TAACCAGTAT TTGCAACCCA CACTGCCCCC |
| | TGTTGTCATA |
| 361 | GGTCCGTTTT TTGATGGCAG CATTCGA |

In certain embodiments, the disclosure relates to heteromultimers that comprise at least one ALK3 polypeptide, which includes fragments, functional variants, and modified forms thereof. Preferably, ALK3 polypeptides for use in accordance with the disclosure (e.g., heteromultimers comprising an ALK3 polypeptide and uses thereof) are soluble (e.g., an extracellular domain of ALK3). In other preferred embodiments, ALK3 polypeptides for use in accordance with the disclosure bind to and/or inhibit (antagonize) activity (e.g., Smad signaling) of one or more TGF-beta superfamily ligands. In some embodiments, heteromultimers of the disclosure comprise at least one ALK3 polypeptide that is at least 70%, 75%, 80%, 85%, 90%, 91%, 92%, 93%, 94%, 95%, 96%, 97%, 98%, 99%, or 100% identical to the amino acid sequence of SEQ ID NOs: 22 or 23. In some embodiments, heteromultimers of the disclosure comprise at least one ALK3 polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 24-61 (e.g., amino acid residues 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, or 61) of SEQ ID NO: 22, and ends at any one of amino acids 130-152 (e.g., amino acid residues 130, 131, 132, 133, 134, 135, 136, 137, 138, 139, 140, 141, 142, 143, 144, 145, 146, 147, 148, 149, 150, 151, 152) of SEQ ID NO: 22. In some embodiments, heteromultimers of the disclosure comprise at least one ALK3 polypeptide that is at least 70%, 75%, 80%, 85%, 90%, 91%, 92%, 93%, 94%, 95%, 96%, 97%, 98%, 99%, or 100% identical to amino acids of 24-130 of SEQ ID NO: 22. In some embodiments, heteromultimers of the disclosure comprise at least one ALK3 polypeptide that is at least 70%, 75%, 80%, 85%, 90%, 91%, 92%, 93%, 94%, 95%, 96%, 97%, 98%, 99%, or 100% identical to amino acids of 24-152 of SEQ ID NO: 22. In some embodiments, heteromultimers of the disclosure comprise at least one ALK3 polypeptide that is at least 70%, 75%, 80%, 85%, 90%, 91%, 92%, 93%, 94%, 95%, 96%, 97%, 98%, 99%, or 100% identical to amino acids of 61-130 of SEQ ID NO: 22. In some embodiments, heteromultimers of the disclosure comprise at least one ALK3 polypeptide that is at least 70%, 75%, 80%, 85%, 90%, 91%, 92%, 93%, 94%, 95%, 96%, 97%, 98%, 99%, or 100% identical to amino acids of 61-152 of SEQ ID NO: 22.

In certain aspects, the present disclosure relates to heteromultimers that comprise an ALK4 polypeptide. As used herein, the term “ALK4” refers to a family of activin receptor-like kinase-4 proteins from any species and variants derived from such ALK4 proteins by mutagenesis or other modification. Reference to ALK4 herein is understood to be a reference to any one of the currently identified forms. Members of the ALK4 family are generally transmembrane proteins, composed of a ligand-binding extracellular domain with a cysteine-rich region, a transmembrane domain, and a cytoplasmic domain with predicted serine/threonine kinase activity.

The term “ALK4 polypeptide” includes polypeptides comprising any naturally occurring polypeptide of an ALK4 family member as well as any variants thereof (including mutants, fragments, fusions, and peptidomimetic forms) that retain a useful activity.

The human ALK4 precursor protein, isoform A sequence (NCBI Ref Seq NP_004293) is as follows:

| (SEQ ID NO: 26) | |
|---|---|
| 1 | MAESAGASSF FPLVVLLLAG SGGSGPRGVQ ALLCACTSCL QANYTCETDG |
| | ACMVSIFNLD |
| 61 | GMEHHVRTCI PKVELVPAGK PFYCLSSEDL RNTHCCYTDY CNRIDLRVPS |
| | GHLKEPEHPS |
| 121 | MWGPVELVGI IAGPVFLLFL IIIIVFLVIN YHQRVYHNRQ RLDMEDPSCE |
| | MCLSKDKTLQ |
| 181 | DLVYDLSTSG SGSGLPLFVQ RTVARTIVLQ EIIGKGRFGE VWRGRWRGGD |
| | VAVKIFSSRE |
| 241 | ERSWFREAEI YQTVMLRHEN ILGFIAADNK DNGTWTQLWL VSDYHEHGSL |
| | FDYLNRYTVT |
| 301 | IEGMIKLALS AASGLAHLHM EIVGTQGKPG IAHRDLKSKN ILVKKNGMCA |
| | IADLGLAVRH |
| 361 | DAVTDTIDIA PNQRVGTKRY MAPEVLDETI NMKHFDSFKC ADIYALGLVY |
| | WEIARRCNSG |
| 421 | GVHEEYQLPY YDLVPSDPSI EEMRKVVCDQ KLRPNIPNWW QSYEALRVMG |
| | KMMRECWYAN |
| 481 | GAARLTALRI KKTLSQLSVQ EDVKI |

The signal peptide is indicated by a single underline and the extracellular domain is indicated in bold font.

A processed extracellular human ALK4 polypeptide sequence is as follows:

(SEQ ID NO: 27)
SGPRGVQALLCACTSCLQANYTCETDGACMVSIFNLDGMEHHVRTCIPKV
ELVPAGKPFYCLSSEDLRNTHCCYTDYCNRIDLRVPSGHLKEPEHPSMWG
PVE

A nucleic acid sequence encoding the ALK4 precursor protein is shown below (SEQ ID NO: 28), corresponding to nucleotides 78-1592 of Genbank Reference Sequence NM_004302.4. The signal sequence is underlined and the extracellular domain is indicated in bold font.

(SEQ ID NO: 28)
ATGGCGGAGTCGGCCGGAGCCTCCTCCTTCTTCCCCCTTGTTGTCCTCCT
GCTCGCCGGCAGCGGCGGGTCCGGGCCCCGGGGGGTCCAGGCTCTGCTGT
GTGCGTGCACCAGCTGCCTCCAGGCCAACTACACGTGTGAGACAGATGGG
GCCTGCATGGTTTCCATTTTCAATCTGGATGGGATGGAGCACCATGTGCG
CACCTGCATCCCCAAAGTGGAGCTGGTCCCTGCCGGGAAGCCCTTCTACT
GCCTGAGCTCGGAGGACCTGCGCAACACCCACTGCTGCTACACTGACTAC
TGCAACAGGATCGACTTGAGGGTGCCCAGTGGTCACCTCAAGGAGCCTGA
GCACCCGTCCATGTGGGGCCCGGTGGAGCTGGTAGGCATCATCGCCGGCC
CGGTGTTCCTCCTGTTCCTCATCATCATCATTGTTTTCCTTGTCATTAAC
TATCATCAGCGTGTCTATCACAACCGCCAGAGACTGGACATGGAAGATCC
CTCATGTGAGATGTGTCTCTCCAAAGACAAGACGCTCCAGGATCTTGTCT
ACGATCTCTCCACCTCAGGGTCTGGCTCAGGGTTACCCCTCTTTGTCCAG
CGCACAGTGGCCCGAACCATCGTTTTACAAGAGATTATTGGCAAGGGTCG
GTTTGGGGAAGTATGGCGGGGCCGCTGGAGGGGTGGTGATGTGGCTGTGA
AAATATTCTCTTCTCGTGAAGAACGGTCTTGGTTCAGGGAAGCAGAGATA
TACCAGACGGTCATGCTGCGCCATGAAAACATCCTTGGATTTATTGCTGC
TGACAATAAAGATAATGGCACCTGGACACAGCTGTGGCTTGTTTCTGACT
ATCATGAGCACGGGTCCCTGTTTGATTATCTGAACCGGTACACAGTGACA
ATTGAGGGGATGATTAAGCTGGCCTTGTCTGCTGCTAGTGGGCTGGCACA
CCTGCACATGGAGATCGTGGGCACCCAAGGGAAGCCTGGAATTGCTCATC
GAGACTTAAAGTCAAAGAACATTCTGGTGAAGAAAAATGGCATGTGTGCC
ATAGCAGACCTGGGCCTGGCTGTCCGTCATGATGCAGTCACTGACACCAT
TGACATTGCCCCGAATCAGAGGGTGGGGACCAAACGATACATGGCCCCTG
AAGTACTTGATGAAACCATTAATATGAAACACTTTGACTCCTTTAAATGT
GCTGATATTTATGCCCTCGGGCTTGTATATTGGGAGATTGCTCGAAGATG
CAATTCTGGAGGAGTCCATGAAGAATATCAGCTGCCATATTACGACTTAG
TGCCCTCTGACCCTTCCATTGAGGAAATGCGAAAGGTTGTATGTGATCAG
AAGCTGCGTCCCAACATCCCCAACTGGTGGCAGAGTTATGAGGCACTGCG
GGTGATGGGGAAGATGATGCGAGAGTGTTGGTATGCCAACGGCGCAGCCC
GCCTGACGGCCCTGCGCATCAAGAAGACCCTCTCCCAGCTCAGCGTGCAG
GAAGACGTGAAGATC

A nucleic acid sequence encoding an extracellular ALK4 polypeptide is as follows:

(SEQ ID NO: 29)
TCCGGGCCCCGGGGGGTCCAGGCTCTGCTGTGTGCGTGCACCAGCTGCCT
CCAGGCCAACTACACGTGTGAGACAGATGGGGCCTGCATGGTTTCCATTT
TCAATCTGGATGGGATGGAGCACCATGTGCGCACCTGCATCCCCAAAGTG
GAGCTGGTCCCTGCCGGGAAGCCCTTCTACTGCCTGAGCTCGGAGGACCT
GCGCAACACCCACTGCTGCTACACTGACTACTGCAACAGGATCGACTTGA
GGGTGCCCAGTGGTCACCTCAAGGAGCCTGAGCACCCGTCCATGTGGGGC
CCGGTGGAG

An alternative isoform of human ALK4 precursor protein sequence, isoform C (NCBI Ref Seq NP_064733.3), is as follows:

| (SEQ ID NO: 83) | |
|---|---|
| 1 | MAESAGASSF FPLVVLLLAG SGGSGPRGVQ ALLCACTSCL QANYTCETDG |
| | ACMVSIFNLD |
| 61 | GMEHHVRTCI PKVELVPAGK PFYCLSSEDL RNTHCCYTDY CNRIDLRVPS |
| | GHLKEPEHPS |
| 121 | MWGPVELVGI IAGPVFLLFL IIIIVFLVIN YHQRVYHNRQ RLDMEDPSCE |
| | MCLSKDKTLQ |
| 181 | DLVYDLSTSG SGSGLPLFVQ RTVARTIVLQ EIIGKGRFGE VWRGRWRGGD |
| | VAVKIFSSRE |
| 241 | ERSWFREAEI YQTVMLRHEN ILGFIAADNK ADCSFLTLPW EVVMVSAAPK |
| | LRSLRLQYKG |
| 301 | GRGRARFLFP LNNGTWTQLW LVSDYHEHGS LFDYLNRYTV TIEGMIKLAL |
| | SAASGLAHLH |
| 361 | MEIVGTQGKP GIAHRDLKSK NILVKKNGMC AIADLGLAVR HDAVTDTIDI |
| | APNQRVGTKR |
| 421 | YMAPEVLDET INMKHFDSFK CADIYALGLV YWEIARRCNS GGVHEEYQLP |
| | YYDLVPSDPS |
| 481 | IEEMRKVVCD QKLRPNIPNW WQSYEALRVM GKMMRECWYA NGAARLTALR |
| | IKKTLSQLSV |
| 541 | QEDVKI |

The signal peptide is indicated by a single underline and the extracellular domain is indicated in bold font.

A processed extracellular ALK4 polypeptide sequence (isoform C) is as follows:

(SEQ ID NO: 84)
SGPRGVQALLCACTSCLQANYTCETDGACMVSIFNLDGMEHHVRTCIPKV
ELVPAGKPFYCLSSEDLRNTHCCYTDYCNRIDLRVPSGHLKEPEHPSMWG
PVE

A nucleic acid sequence encoding the ALK4 precursor protein (isoform C) is shown below (SEQ ID NO: 85), corresponding to nucleotides 78-1715 of Genbank Reference Sequence NM_020328.3. The signal sequence is underlined and the extracellular domain is indicated in bold font.

(SEQ ID NO: 85)
ATGGCGGAGTCGGCCGGAGCCTCCTCCTTCTTCCCCCTTGTTGTCCTCCT
GCTCGCCGGCAGCGGCGGGTCCGGGCCCCGGGGGGTCCAGGCTCTGCTGT
GTGCGTGCACCAGCTGCCTCCAGGCCAACTACACGTGTGAGACAGATGGG
GCCTGCATGGTTTCCATTTTCAATCTGGATGGGATGGAGCACCATGTGCG
CACCTGCATCCCCAAAGTGGAGCTGGTCCCTGCCGGGAAGCCCTTCTACT
GCCTGAGCTCGGAGGACCTGCGCAACACCCACTGCTGCTACACTGACTAC
TGCAACAGGATCGACTTGAGGGTGCCCAGTGGTCACCTCAAGGAGCCTGA
GCACCCGTCCATGTGGGGCCCGGTGGAGCTGGTAGGCATCATCGCCGGCC
CGGTGTTCCTCCTGTTCCTCATCATCATCATTGTTTTCCTTGTCATTAAC
TATCATCAGCGTGTCTATCACAACCGCCAGAGACTGGACATGGAAGATCC
CTCATGTGAGATGTGTCTCTCCAAAGACAAGACGCTCCAGGATCTTGTCT
ACGATCTCTCCACCTCAGGGTCTGGCTCAGGGTTACCCCTCTTTGTCCAG
CGCACAGTGGCCCGAACCATCGTTTTACAAGAGATTATTGGCAAGGGTCG
GTTTGGGGAAGTATGGCGGGGCCGCTGGAGGGGTGGTGATGTGGCTGTGA
AAATATTCTCTTCTCGTGAAGAACGGTCTTGGTTCAGGGAAGCAGAGATA
TACCAGACGGTCATGCTGCGCCATGAAAACATCCTTGGATTTATTGCTGC
TGACAATAAAGCAGACTGCTCATTCCTCACATTGCCATGGGAAGTTGTAA
TGGTCTCTGCTGCCCCCAAGCTGAGGAGCCTTAGACTCCAATACAAGGGA
GGAAGGGGAAGAGCAAGATTTTTATTCCCACTGAATAATGGCACCTGGAC
ACAGCTGTGGCTTGTTTCTGACTATCATGAGCACGGGTCCCTGTTTGATT
ATCTGAACCGGTACACAGTGACAATTGAGGGGATGATTAAGCTGGCCTTG
TCTGCTGCTAGTGGGCTGGCACACCTGCACATGGAGATCGTGGGCACCCA
AGGGAAGCCTGGAATTGCTCATCGAGACTTAAAGTCAAAGAACATTCTGG
TGAAGAAAAATGGCATGTGTGCCATAGCAGACCTGGGCCTGGCTGTCCGT
CATGATGCAGTCACTGACACCATTGACATTGCCCCGAATCAGAGGGTGGG
GACCAAACGATACATGGCCCCTGAAGTACTTGATGAAACCATTAATATGA
AACACTTTGACTCCTTTAAATGTGCTGATATTTATGCCCTCGGGCTTGTA
TATTGGGAGATTGCTCGAAGATGCAATTCTGGAGGAGTCCATGAAGAATA
TCAGCTGCCATATTACGACTTAGTGCCCTCTGACCCTTCCATTGAGGAAA
TGCGAAAGGTTGTATGTGATCAGAAGCTGCGTCCCAACATCCCCAACTGG
TGGCAGAGTTATGAGGCACTGCGGGTGATGGGGAAGATGATGCGAGAGTG
TTGGTATGCCAACGGCGCAGCCCGCCTGACGGCCCTGCGCATCAAGAAGA
CCCTCTCCCAGCTCAGCGTGCAGGAAGACGTGAAGATC

A nucleic acid sequence encoding an extracellular ALK4 polypeptide (isoform C) is as follows:

(SEQ ID NO: 86)
TCCGGGCCCCGGGGGGTCCAGGCTCTGCTGTGTGCGTGCACCAGCTGCCT
CCAGGCCAACTACACGTGTGAGACAGATGGGGCCTGCATGGTTTCCATTT
TCAATCTGGATGGGATGGAGCACCATGTGCGCACCTGCATCCCCAAAGTG
GAGCTGGTCCCTGCCGGGAAGCCCTTCTACTGCCTGAGCTCGGAGGACCT
GCGCAACACCCACTGCTGCTACACTGACTACTGCAACAGGATCGACTTGA
GGGTGCCCAGTGGTCACCTCAAGGAGCCTGAGCACCCGTCCATGTGGGGC
CCGGTGGAG

In certain embodiments, the disclosure relates to heteromultimers that comprise at least one ALK4 polypeptide, which includes fragments, functional variants, and modified forms thereof. Preferably, ALK4 polypeptides for use in accordance with the disclosure (e.g., heteromultimers comprising an ALK4 polypeptide and uses thereof) are soluble (e.g., an extracellular domain of ALK4). In other preferred embodiments, ALK4 polypeptides for use in accordance with the disclosure bind to and/or inhibit (antagonize) activity (e.g., Smad signaling) of one or more TGF-beta superfamily ligands. In some embodiments, heteromultimers of the disclosure comprise at least one ALK4 polypeptide that is at least 70%, 75%, 80%, 85%, 90%, 91%, 92%, 93%, 94%, 95%, 96%, 97%, 98%, 99%, or 100% identical to the amino acid sequence of SEQ ID NOs: 26, 27, 83, or 84. In some embodiments, heteromultimers of the disclosure comprise at least one ALK4 polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 24-34 (e.g., amino acid residues 24, 25, 26, 27, 28, 29, 30, 31, 32, 33, or 34) of SEQ ID NO: 26, and ends at any one of amino acids 101-126 (e.g., amino acid residues 101, 102, 103, 104, 105, 106, 107, 108, 109, 110, 111, 112, 113, 114, 115, 116, 117, 118, 119, 120, 121, 122, 123, 124, 125, or 126) of SEQ ID NO: 26. In some embodiments, heteromultimers of the disclosure comprise at least one ALK4 polypeptide that is at least 70%, 75%, 80%, 85%, 90%, 91%, 92%, 93%, 94%, 95%, 96%, 97%, 98%, 99%, or 100% identical to amino acids of 24-101 of SEQ ID NO: 26. In some embodiments, heteromultimers of the disclosure comprise at least one ALK4 polypeptide that is at least 70%, 75%, 80%, 85%, 90%, 91%, 92%, 93%, 94%, 95%, 96%, 97%, 98%, 99%, or 100% identical to amino acids of 24-126 of SEQ ID NO: 26. In some embodiments, heteromultimers of the disclosure comprise at least one ALK4 polypeptide that is at least 70%, 75%, 80%, 85%, 90%, 91%, 92%, 93%, 94%, 95%, 96%, 97%, 98%, 99%, or 100% identical to amino acids of 34-101 of SEQ ID NO: 26. In some embodiments, heteromultimers of the disclosure comprise at least one ALK4 polypeptide that is at least 70%, 75%, 80%, 85%, 90%, 91%, 92%, 93%, 94%, 95%, 96%, 97%, 98%, 99%, or 100% identical to amino acids of 34-126 of SEQ ID NO: 26. In some embodiments, heteromultimers of the disclosure comprise at least one ALK4 polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 24-34 (e.g., amino acid residues 24, 25, 26, 27, 28, 29, 30, 31, 32, 33, or 34) of SEQ ID NO: 83, and ends at any one of amino acids 101-126 (e.g., amino acid residues 101, 102, 103, 104, 105, 106, 107, 108, 109, 110, 111, 112, 113, 114, 115, 116, 117, 118, 119, 120, 121, 122, 123, 124, 125, or 126) of SEQ ID NO: 83. In some embodiments, heteromultimers of the disclosure comprise at least one ALK4 polypeptide that is at least 70%, 75%, 80%, 85%, 90%, 91%, 92%, 93%, 94%, 95%, 96%, 97%, 98%, 99%, or 100% identical to amino acids of 24-101 of SEQ ID NO: 83. In some embodiments, heteromultimers of the disclosure comprise at least one ALK4 polypeptide that is at least 70%, 75%, 80%, 85%, 90%, 91%, 92%, 93%, 94%, 95%, 96%, 97%, 98%, 99%, or 100% identical to amino acids of 24-126 of SEQ ID NO: 83. In some embodiments, heteromultimers of the disclosure comprise at least one ALK4 polypeptide that is at least 70%, 75%, 80%, 85%, 90%, 91%, 92%, 93%, 94%, 95%, 96%, 97%, 98%, 99%, or 100% identical to amino acids of 34-101 of SEQ ID NO: 83. In some embodiments, heteromultimers of the disclosure comprise at least one ALK4 polypeptide that is at least 70%, 75%, 80%, 85%, 90%, 91%, 92%, 93%, 94%, 95%, 96%, 97%, 98%, 99%, or 100% identical to amino acids of 34-126 of SEQ ID NO: 83.

In certain aspects, the present disclosure relates to heteromultimers that comprise an ALK5 polypeptide. As used herein, the term “ALK5” refers to a family of activin receptor-like kinase-5 proteins from any species and variants derived from such ALK4 proteins by mutagenesis or other modification. Reference to ALK5 herein is understood to be a reference to any one of the currently identified forms. Members of the ALK5 family are generally transmembrane proteins, composed of a ligand-binding extracellular domain with a cysteine-rich region, a transmembrane domain, and a cytoplasmic domain with predicted serine/threonine kinase activity.

The term “ALK5 polypeptide” includes polypeptides comprising any naturally occurring polypeptide of an ALK5 family member as well as any variants thereof (including mutants, fragments, fusions, and peptidomimetic forms) that retain a useful activity.

The human ALK5 precursor protein, isoform 1 sequence (NCBI Ref Seq NP_004603.1) is as follows:

| (SEQ ID NO: 30) | |
|---|---|
| 1 | MEAAVAAPRP RLLLLVLAAA AAAAAALLPG ATALQCFCHL CTKDNFTCVT |
| | DGLCFVSVTE |
| 61 | TTDKVIHNSM CIAEIDLIPR DRPFVCAPSS KTGSVTTTYC CNQDHCNKIE |
| | LPTTVKSSPG |
| 121 | LGPVELAAVI AGPVCFVCIS LMLMVYICHN RTVIHHRVPN EEDPSLDRPF |
| | ISEGTTLKDL |
| 181 | IYDMTTSGSG SGLPLLVQRT IARTIVLQES IGKGRFGEVW RGKWRGEEVA |
| | VKIFSSREER |
| 241 | SWFREAEIYQ TVMLRHENIL GFIAADNKDN GTWTQLWLVS DYHEHGSLFD |
| | YLNRYTVTVE |
| 301 | GMIKLALSTA SGLAHLHMEI VGTQGKPAIA HRDLKSKNIL VKKNGTCCIA |
| | DLGLAVRHDS |
| 361 | ATDTIDIAPN HRVGTKRYMA PEVLDDSINM KHFESFKRAD IYAMGLVFWE |
| | IARRCSIGGI |
| 421 | HEDYQLPYYD LVPSDPSVEE MRKVVCEQKL RPNIPNRWQS CEALRVMAKI |
| | MRECWYANGA |
| 481 | ARLTALRIKK TLSQLSQQEG IKM |

The signal peptide is indicated by a single underline and the extracellular domain is indicated in bold font.

A processed extracellular ALK5 polypeptide sequence is as follows:

(SEQ ID NO: 31)
AALLPGATALQCFCHLCTKDNFTCVTDGLCFVSVTETTDKVIHNSMCIAE
IDLIPRDRPFVCAPSSKTGSVTTTYCCNQDHCNKIELPTTVKSSPGLGPV
EL

A nucleic acid sequence encoding the ALK5 precursor protein is shown below (SEQ ID NO: 32), corresponding to nucleotides 77-1585 of Genbank Reference Sequence NM_004612.2. The signal sequence is underlined and the extracellular domain is indicated in bold font.

(SEQ ID NO: 32)
ATGGAGGCGGCGGTCGCTGCTCCGCGTCCCCGGCTGCTCCTCCTCGTGCT
GGCGGCGGCGGCGGCGGCGGCGGCGGCGCTGCTCCCGGGGGCGACGGCGT
TACAGTGTTTCTGCCACCTCTGTACAAAAGACAATTTTACTTGTGTGACA
GATGGGCTCTGCTTTGTCTCTGTCACAGAGACCACAGACAAAGTTATACA
CAACAGCATGTGTATAGCTGAAATTGACTTAATTCCTCGAGATAGGCCGT
TTGTATGTGCACCCTCTTCAAAAACTGGGTCTGTGACTACAACATATTGC
TGCAATCAGGACCATTGCAATAAAATAGAACTTCCAACTACTGTAAAGTC
ATCACCTGGCCTTGGTCCTGTGGAACTGGCAGCTGTCATTGCTGGACCAG
TGTGCTTCGTCTGCATCTCACTCATGTTGATGGTCTATATCTGCCACAAC
CGCACTGTCATTCACCATCGAGTGCCAAATGAAGAGGACCCTTCATTAGA
TCGCCCTTTTATTTCAGAGGGTACTACGTTGAAAGACTTAATTTATGATA
TGACAACGTCAGGTTCTGGCTCAGGTTTACCATTGCTTGTTCAGAGAACA
ATTGCGAGAACTATTGTGTTACAAGAAAGCATTGGCAAAGGTCGATTTGG
AGAAGTTTGGAGAGGAAAGTGGCGGGGAGAAGAAGTTGCTGTTAAGATAT
TCTCCTCTAGAGAAGAACGTTCGTGGTTCCGTGAGGCAGAGATTTATCAA
ACTGTAATGTTACGTCATGAAAACATCCTGGGATTTATAGCAGCAGACAA
TAAAGACAATGGTACTTGGACTCAGCTCTGGTTGGTGTCAGATTATCATG
AGCATGGATCCCTTTTTGATTACTTAAACAGATACACAGTTACTGTGGAA
GGAATGATAAAACTTGCTCTGTCCACGGCGAGCGGTCTTGCCCATCTTCA
CATGGAGATTGTTGGTACCCAAGGAAAGCCAGCCATTGCTCATAGAGATT
TGAAATCAAAGAATATCTTGGTAAAGAAGAATGGAACTTGCTGTATTGCA
GACTTAGGACTGGCAGTAAGACATGATTCAGCCACAGATACCATTGATAT
TGCTCCAAACCACAGAGTGGGAACAAAAAGGTACATGGCCCCTGAAGTTC
TCGATGATTCCATAAATATGAAACATTTTGAATCCTTCAAACGTGCTGAC
ATCTATGCAATGGGCTTAGTATTCTGGGAAATTGCTCGACGATGTTCCAT
TGGTGGAATTCATGAAGATTACCAACTGCCTTATTATGATCTTGTACCTT
CTGACCCATCAGTTGAAGAAATGAGAAAAGTTGTTTGTGAACAGAAGTTA
AGGCCAAATATCCCAAACAGATGGCAGAGCTGTGAAGCCTTGAGAGTAAT
GGCTAAAATTATGAGAGAATGTTGGTATGCCAATGGAGCAGCTAGGCTTA
CAGCATTGCGGATTAAGAAAACATTATCGCAACTCAGTCAACAGGAAGGC
ATCAAAATG

A nucleic acid sequence encoding an extracellular human ALK5 polypeptide is as follows:

(SEQ ID NO: 33)

GCGGCGCTGCTCCCGGGGGCGACGGCGTTACAGTGTTTCTGCCACCTCTG
TACAAAAGACAATTTTACTTGTGTGACAGATGGGCTCTGCTTTGTCTCTG
TCACAGAGACCACAGACAAAGTTATACACAACAGCATGTGTATAGCTGAA
ATTGACTTAATTCCTCGAGATAGGCCGTTTGTATGTGCACCCTCTTCAAA
AACTGGGTCTGTGACTACAACATATTGCTGCAATCAGGACCATTGCAATA
AAATAGAACTTCCAACTACTGTAAAGTCATCACCTGGCCTTGGTCCTGTG
GAACTG

An alternative isoform of the human ALK5 precursor protein sequence, isoform 2 (NCBI Ref Seq XP 005252207.1), is as follows:

| (SEQ ID NO: 87) | |
|---|---|
| 1 | MEAAVAAPRP RLLLLVLAAA AAAAAALLPG ATALQCFCHL CTKDNFTCVT |
| | DGLCFVSVTE |
| 61 | TTDKVIHNSM CIAEIDLIPR DRPFVCAPSS KTGSVTTTYC CNQDHCNKIE |
| | LPTTGPFSVK |
| 121 | SSPGLGPVEL AAVIAGPVCF VCISLMLMVY ICHNRTVIHH RVPNEEDPSL |
| | DRPFISEGTT |
| 181 | LKDLIYDMTT SGSGSGLPLL VQRTIARTIV LQESIGKGRF GEVWRGKWRG |
| | EEVAVKIFSS |
| 241 | REERSWFREA EIYQTVMLRH ENILGFIAAD NKDNGTWTQL WLVSDYHEHG |
| | SLFDYLNRYT |
| 301 | VTVEGMIKLA LSTASGLAHL HMEIVGTQGK PAIAHRDLKS KNILVKKNGT |
| | CCIADLGLAV |
| 361 | RHDSATDTID IAPNHRVGTK RYMAPEVLDD SINMKHFESF KRADIYAMGL |
| | VFWEIARRCS |
| 421 | IGGIHEDYQL PYYDLVPSDP SVEEMRKVVC EQKLRPNIPN RWQSCEALRV |
| | MAKIMRECWY |
| 481 | ANGAARLTAL RIKKTLSQLS QQEGIKM |

The signal peptide is indicated by a single underline and the extracellular domain is indicated in bold font.

A processed extracellular ALK5 polypeptide sequence (isoform 2) is as follows:

(SEQ ID NO: 88)

AALLPGATALQCFCHLCTKDNFTCVTDGLCFVSVTETTDKVIHNSMCIAE
IDLIPRDRPFVCAPSSKTGSVTTTYCCNQDHCNKIELPTTGPFSVKSSPG
LGPVEL

A nucleic acid sequence encoding human ALK5 precursor protein (isoform 2) is shown below (SEQ ID NO: 89), corresponding to nucleotides 77-1597 of Genbank Reference Sequence XM_005252150.1. The signal sequence is underlined and the extracellular domain is indicated in bold font.

(SEQ ID NO: 89)

ATGGAGGCGGCGGTCGCTGCTCCGCGTCCCCGGCTGCTCCTCCTCGTGCT
GGCGGCGGCGGCGGCGGCGGCGGCGGCGCTGCTCCCGGGGGCGACGGCGT
TACAGTGTTTCTGCCACCTCTGTACAAAAGACAATTTTACTTGTGTGACA
GATGGGCTCTGCTTTGTCTCTGTCACAGAGACCACAGACAAAGTTATACA
CAACAGCATGTGTATAGCTGAAATTGACTTAATTCCTCGAGATAGGCCGT
TTGTATGTGCACCCTCTTCAAAAACTGGGTCTGTGACTACAACATATTGC
TGCAATCAGGACCATTGCAATAAAATAGAACTTCCAACTACTGGCCCTTT
TTCAGTAAAGTCATCACCTGGCCTTGGTCCTGTGGAACTGGCAGCTGTCA
TTGCTGGACCAGTGTGCTTCGTCTGCATCTCACTCATGTTGATGGTCTAT
ATCTGCCACAACCGCACTGTCATTCACCATCGAGTGCCAAATGAAGAGGA
CCCTTCATTAGATCGCCCTTTTATTTCAGAGGGTACTACGTTGAAAGACT
TAATTTATGATATGACAACGTCAGGTTCTGGCTCAGGTTTACCATTGCTT
GTTCAGAGAACAATTGCGAGAACTATTGTGTTACAAGAAAGCATTGGCAA
AGGTCGATTTGGAGAAGTTTGGAGAGGAAAGTGGCGGGGAGAAGAAGTTG
CTGTTAAGATATTCTCCTCTAGAGAAGAACGTTCGTGGTTCCGTGAGGCA
GAGATTTATCAAACTGTAATGTTACGTCATGAAAACATCCTGGGATTTAT
AGCAGCAGACAATAAAGACAATGGTACTTGGACTCAGCTCTGGTTGGTGT
CAGATTATCATGAGCATGGATCCCTTTTTGATTACTTAAACAGATACACA
GTTACTGTGGAAGGAATGATAAAACTTGCTCTGTCCACGGCGAGCGGTCT
TGCCCATCTTCACATGGAGATTGTTGGTACCCAAGGAAAGCCAGCCATTG
CTCATAGAGATTTGAAATCAAAGAATATCTTGGTAAAGAAGAATGGAACT
TGCTGTATTGCAGACTTAGGACTGGCAGTAAGACATGATTCAGCCACAGA
TACCATTGATATTGCTCCAAACCACAGAGTGGGAACAAAAAGGTACATGG
CCCCTGAAGTTCTCGATGATTCCATAAATATGAAACATTTTGAATCCTTC
AAACGTGCTGACATCTATGCAATGGGCTTAGTATTCTGGGAAATTGCTCG
ACGATGTTCCATTGGTGGAATTCATGAAGATTACCAACTGCCTTATTATG
ATCTTGTACCTTCTGACCCATCAGTTGAAGAAATGAGAAAAGTTGTTTGT
GAACAGAAGTTAAGGCCAAATATCCCAAACAGATGGCAGAGCTGTGAAGC
CTTGAGAGTAATGGCTAAAATTATGAGAGAATGTTGGTATGCCAATGGAG
CAGCTAGGCTTACAGCATTGCGGATTAAGAAAACATTATCGCAACTCAGT
CAACAGGAAGGCATCAAAATG

A nucleic acid sequence encoding an processed extracellular ALK5 polypeptide is as follows:

(SEQ ID NO: 90)

GCGGCGCTGCTCCCGGGGGCGACGGCGTTACAGTGTTTCTGCCACCTCTG
TACAAAAGACAATTTTACTTGTGTGACAGATGGGCTCTGCTTTGTCTCTG
TCACAGAGACCACAGACAAAGTTATACACAACAGCATGTGTATAGCTGAA
ATTGACTTAATTCCTCGAGATAGGCCGTTTGTATGTGCACCCTCTTCAAA
AACTGGGTCTGTGACTACAACATATTGCTGCAATCAGGACCATTGCAATA
AAATAGAACTTCCAACTACTGGCCCTTTTTCAGTAAAGTCATCACCTGGC
CTTGGTCCTGTGGAACTG

In certain embodiments, the disclosure relates to heteromultimers that comprise at least one ALK5 polypeptide, which includes fragments, functional variants, and modified forms thereof. Preferably, ALK5 polypeptides for use in accordance with the disclosure (e.g., heteromultimers comprising an ALK5 polypeptide and uses thereof) are soluble (e.g., an extracellular domain of ALK5). In other preferred embodiments, ALK5 polypeptides for use in accordance with the disclosure bind to and/or inhibit (antagonize) activity (e.g., Smad signaling) of one or more TGF-beta superfamily ligands. In some embodiments, heteromultimers of the disclosure comprise at least one ALK5 polypeptide that is at least 70%, 75%, 80%, 85%, 90%, 91%, 92%, 93%, 94%, 95%, 96%, 97%, 98%, 99%, or 100% identical to the amino acid sequence of SEQ ID NOs: 30, 31, 87, or 88. In some embodiments, heteromultimers of the disclosure comprise at least one ALK5 polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 25-36 (e.g., amino acid residues 25, 26, 27, 28, 29, 30, 31, 32, 33, 34, 35, or 36) of SEQ ID NO: 30, and ends at any one of amino acids 101-126 (e.g., amino acid residues 101, 102, 103, 104, 105, 106, 107, 108, 109, 110, 111, 112, 113, 114, 115, 116, 117, 118, 119, 120, 121, 122, 123, 124, 125, or 126) of SEQ ID NO: 30. In some embodiments, heteromultimers of the disclosure comprise at least one ALK5 polypeptide that is at least 70%, 75%, 80%, 85%, 90%, 91%, 92%, 93%, 94%, 95%, 96%, 97%, 98%, 99%, or 100% identical to amino acids of 25-101 of SEQ ID NO: 30. In some embodiments, heteromultimers of the disclosure comprise at least one ALK5 polypeptide that is at least 70%, 75%, 80%, 85%, 90%, 91%, 92%, 93%, 94%, 95%, 96%, 97%, 98%, 99%, or 100% identical to amino acids of 25-126 of SEQ ID NO: 30. In some embodiments, heteromultimers of the disclosure comprise at least one ALK5 polypeptide that is at least 70%, 75%, 80%, 85%, 90%, 91%, 92%, 93%, 94%, 95%, 96%, 97%, 98%, 99%, or 100% identical to amino acids of 36-101 of SEQ ID NO: 30. In some embodiments, heteromultimers of the disclosure comprise at least one ALK5 polypeptide that is at least 70%, 75%, 80%, 85%, 90%, 91%, 92%, 93%, 94%, 95%, 96%, 97%, 98%, 99%, or 100% identical to amino acids of 36-126 of SEQ ID NO: 30. In some embodiments, heteromultimers of the disclosure comprise at least one ALK5 polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 25-36 (e.g., amino acid residues 25, 26, 27, 28, 29, 30, 31, 32, 33, 34, 35, or 36) of SEQ ID NO: 87, and ends at any one of amino acids 101-130 (e.g., amino acid residues 101, 102, 103, 104, 105, 106, 107, 108, 109, 110, 111, 112, 113, 114, 115, 116, 117, 118, 119, 120, 121, 122, 123, 124, 125, 126, 127, 128, 129 or 130) of SEQ ID NO: 87. In some embodiments, heteromultimers of the disclosure comprise at least one ALK5 polypeptide that is at least 70%, 75%, 80%, 85%, 90%, 91%, 92%, 93%, 94%, 95%, 96%, 97%, 98%, 99%, or 100% identical to amino acids of 25-101 of SEQ ID NO: 87. In some embodiments, heteromultimers of the disclosure comprise at least one ALK5 polypeptide that is at least 70%, 75%, 80%, 85%, 90%, 91%, 92%, 93%, 94%, 95%, 96%, 97%, 98%, 99%, or 100% identical to amino acids of 25-130 of SEQ ID NO: 87. In some embodiments, heteromultimers of the disclosure comprise at least one ALK5 polypeptide that is at least 70%, 75%, 80%, 85%, 90%, 91%, 92%, 93%, 94%, 95%, 96%, 97%, 98%, 99%, or 100% identical to amino acids of 36-101 of SEQ ID NO: 87. In some embodiments, heteromultimers of the disclosure comprise at least one ALK5 polypeptide that is at least 70%, 75%, 80%, 85%, 90%, 91%, 92%, 93%, 94%, 95%, 96%, 97%, 98%, 99%, or 100% identical to amino acids of 36-130 of SEQ ID NO: 87.

In certain aspects, the present disclosure relates to heteromultimers that comprise an ALK6 polypeptide. As used herein, the term “ALK6” refers to a family of activin receptor-like kinase-6 proteins from any species and variants derived from such ALK6 proteins by mutagenesis or other modification. Reference to ALK6 herein is understood to be a reference to any one of the currently identified forms. Members of the ALK6 family are generally transmembrane proteins, composed of a ligand-binding extracellular domain with a cysteine-rich region, a transmembrane domain, and a cytoplasmic domain with predicted serine/threonine kinase activity.

The term “ALK6 polypeptide” includes polypeptides comprising any naturally occurring polypeptide of an ALK6 family member as well as any variants thereof (including mutants, fragments, fusions, and peptidomimetic forms) that retain a useful activity.

The human ALK6 precursor protein, isoform 1 sequence (NCBI Ref Seq NP_001194.1) is as follows:

| (SEQ ID NO: 34) | |
|---|---|
| 1 | MLLRSAGKLN VGTKKEDGES TAPTPRPKVL RCKCHHHCPE DSVNNICSTD |
| | GYCFTMIEED |
| 61 | DSGLPVVTSG CLGLEGSDFQ CRDTPIPHQR RSIECCTERN ECNKDLHPTL |
| | PPLKNRDFVD |
| 121 | GPIHHRALLI SVTVCSLLLV LIILFCYFRY KRQETRPRYS IGLEQDETYI |
| | PPGESLRDLI |
| 181 | EQSQSSGSGS GLPLLVQRTI AKQIQMVKQI GKGRYGEVWM GKWRGEKVAV |
| | KVFFTTEEAS |
| 241 | WFRETEIYQT VLMRHENILG FIAADIKGTG SWTQLYLITD YHENGSLYDY |
| | LKSTTLDAKS |
| 301 | MLKLAYSSVS GLCHLHTEIF STQGKPAIAH RDLKSKNILV KKNGTCCIAD |
| | LGLAVKFISD |
| 361 | TNEVDIPPNT RVGTKRYMPP EVLDESLNRN HFQSYIMADM YSFGLILWEV |
| | ARRCVSGGIV |
| 421 | EEYQLPYHDL VPSDPSYEDM REIVCIKKLR PSFPNRWSSD ECLRQMGKLM |
| | TECWAHNPAS |
| 481 | RLTALRVKKT LAKMSESQDI KL |

The signal peptide is indicated by a single underline and the extracellular domain is indicated in bold font.

A processed extracellular ALK6 polypeptide sequence is as follows:

(SEQ ID NO: 35)

KKEDGESTAPTPRPKVLRCKCHHHCPEDSVNNICSTDGYCFTMIEEDDSG
LPVVTSGCLGLEGSDFQCRDTPIPHQRRSIECCTERNECNKDLHPTLPPL
KNRDFVDGPIHHR

A nucleic acid sequence encoding the ALK6 precursor protein is shown below (SEQ ID NO: 36), corresponding to nucleotides 275-1780 of Genbank Reference Sequence NM_001203.2. The signal sequence is underlined and the extracellular domain is indicated in bold font.

(SEQ ID NO: 36)

ATGCTTTTGCGAAGTGCAGGAAAATTAAATGTGGGCACCAAGAAAGAGGA
TGGTGAGAGTACAGCCCCCACCCCCCGTCCAAAGGTCTTGCGTTGTAAAT
GCCACCACCATTGTCCAGAAGACTCAGTCAACAATATTTGCAGCACAGAC
GGATATTGTTTCACGATGATAGAAGAGGATGACTCTGGGTTGCCTGTGGT
CACTTCTGGTTGCCTAGGACTAGAAGGCTCAGATTTTCAGTGTCGGGACA
CTCCCATTCCTCATCAAAGAAGATCAATTGAATGCTGCACAGAAAGGAAC
GAATGTAATAAAGACCTACACCCTACACTGCCTCCATTGAAAAACAGAGA
TTTTGTTGATGGACCTATACACCACAGGGCTTTACTTATATCTGTGACTG
TCTGTAGTTTGCTCTTGGTCCTTATCATATTATTTTGTTACTTCCGGTAT
AAAAGACAAGAAACCAGACCTCGATACAGCATTGGGTTAGAACAGGATGA
AACTTACATTCCTCCTGGAGAATCCCTGAGAGACTTAATTGAGCAGTCTC
AGAGCTCAGGAAGTGGATCAGGCCTCCCTCTGCTGGTCCAAAGGACTATA
GCTAAGCAGATTCAGATGGTGAAACAGATTGGAAAAGGTCGCTATGGGGA
AGTTTGGATGGGAAAGTGGCGTGGCGAAAAGGTAGCTGTGAAAGTGTTCT
TCACCACAGAGGAAGCCAGCTGGTTCAGAGAGACAGAAATATATCAGACA
GTGTTGATGAGGCATGAAAACATTTTGGGTTTCATTGCTGCAGATATCAA
AGGGACAGGGTCCTGGACCCAGTTGTACCTAATCACAGACTATCATGAAA
ATGGTTCCCTTTATGATTATCTGAAGTCCACCACCCTAGACGCTAAATCA
ATGCTGAAGTTAGCCTACTCTTCTGTCAGTGGCTTATGTCATTTACACAC
AGAAATCTTTAGTACTCAAGGCAAACCAGCAATTGCCCATCGAGATCTGA
AAAGTAAAAACATTCTGGTGAAGAAAAATGGAACTTGCTGTATTGCTGAC
CTGGGCCTGGCTGTTAAATTTATTAGTGATACAAATGAAGTTGACATACC
ACCTAACACTCGAGTTGGCACCAAACGCTATATGCCTCCAGAAGTGTTGG
ACGAGAGCTTGAACAGAAATCACTTCCAGTCTTACATCATGGCTGACATG
TATAGTTTTGGCCTCATCCTTTGGGAGGTTGCTAGGAGATGTGTATCAGG
AGGTATAGTGGAAGAATACCAGCTTCCTTATCATGACCTAGTGCCCAGTG
ACCCCTCTTATGAGGACATGAGGGAGATTGTGTGCATCAAGAAGTTACGC
CCCTCATTCCCAAACCGGTGGAGCAGTGATGAGTGTCTAAGGCAGATGGG
AAAACTCATGACAGAATGCTGGGCTCACAATCCTGCATCAAGGCTGACAG
CCCTGCGGGTTAAGAAAACACTTGCCAAAATGTCAGAGTCCCAGGACATT
AAACTC

A nucleic acid sequence encoding a processed extracellular ALK6 polypeptide is as follows:

(SEQ ID NO: 37)

AAGAAAGAGGATGGTGAGAGTACAGCCCCCACCCCCCGTCCAAAGGTCTT
GCGTTGTAAATGCCACCACCATTGTCCAGAAGACTCAGTCAACAATATTT
GCAGCACAGACGGATATTGTTTCACGATGATAGAAGAGGATGACTCTGGG
TTGCCTGTGGTCACTTCTGGTTGCCTAGGACTAGAAGGCTCAGATTTTCA
GTGTCGGGACACTCCCATTCCTCATCAAAGAAGATCAATTGAATGCTGCA
CAGAAAGGAACGAATGTAATAAAGACCTACACCCTACACTGCCTCCATTG
AAAAACAGAGATTTTGTTGATGGACCTATACACCACAGG

An alternative isoform of human ALK6 precursor protein sequence, isoform 2 (NCBI Ref Seq NP_001243722.1) is as follows:

| (SEQ ID NO: 91) | |
|---|---|
| 1 | MGWLEELNWQ LHIFLLILLS MHTRANFLDN MLLRSAGKLN VGTKKEDGES |
| | TAPTPRPKVL |
| 61 | RCKCHHHCPE DSVNNICSTD GYCFTMIEED DSGLPVVTSG CLGLEGSDFQ |
| | CRDTPIPHQR |
| 121 | RSIECCTERN ECNKDLHPTL PPLKNRDFVD GPIHHRALLI SVTVCSLLLV |
| | LIILFCYFRY |
| 181 | KRQETRPRYS IGLEQDETYI PPGESLRDLI EQSQSSGSGS GLPLLVQRTI |
| | AKQIQMVKQI |
| 241 | GKGRYGEVWM GKWRGEKVAV KVFFTTEEAS WFRETEIYQT VLMRHENILG |
| | FIAADIKGTG |
| 301 | SWTQLYLITD YHENGSLYDY LKSTTLDAKS MLKLAYSSVS GLCHLHTEIF |
| | STQGKPAIAH |
| 361 | RDLKSKNILV KKNGTCCIAD LGLAVKFISD TNEVDIPPNT RVGTKRYMPP |
| | EVLDESLNRN |
| 421 | HFQSYIMADM YSFGLILWEV ARRCVSGGIV EEYQLPYHDL VPSDPSYEDM |
| | REIVCIKKLR |
| 481 | PSFPNRWSSD ECLRQMGKLM TECWAHNPAS RLTALRVKKT LAKMSESQDI KL |

The signal peptide is indicated by a single underline and the extracellular domain is indicated in bold font.

A processed extracellular ALK6 polypeptide sequence (isoform 2) is as follows:

(SEQ ID NO: 92)

NFLDNMLLRSAGKLNVGTKKEDGESTAPTPRPKVLRCKCHHHCPEDSVNN
ICSTDGYCFTMIEEDDSGLPVVTSGCLGLEGSDFQCRDTPIPHQRRSIEC
CTERNECNKDLHPTLPPLKNRDFVDGPIHHR

A nucleic acid sequence encoding human ALK6 precursor protein (isoform 2) is shown below, corresponding to nucleotides 22-1617 of Genbank Reference Sequence NM_001256793.1. The signal sequence is underlined and the extracellular domain is indicated in bold font.

(SEQ ID NO: 93)

ATGGGTTGGCTGGAAGAACTAAACTGGCAGCTTCACATTTTCTTGCTCAT
TCTTCTCTCTATGCACACAAGGGCAAACTTCCTTGATAACATGCTTTTGC
GAAGTGCAGGAAAATTAAATGTGGGCACCAAGAAAGAGGATGGTGAGAGT
ACAGCCCCCACCCCCCGTCCAAAGGTCTTGCGTTGTAAATGCCACCACCA
TTGTCCAGAAGACTCAGTCAACAATATTTGCAGCACAGACGGATATTGTT
TCACGATGATAGAAGAGGATGACTCTGGGTTGCCTGTGGTCACTTCTGGT
TGCCTAGGACTAGAAGGCTCAGATTTTCAGTGTCGGGACACTCCCATTCC
TCATCAAAGAAGATCAATTGAATGCTGCACAGAAAGGAACGAATGTAATA
AAGACCTACACCCTACACTGCCTCCATTGAAAAACAGAGATTTTGTTGAT
GGACCTATACACCACAGGGCTTTACTTATATCTGTGACTGTCTGTAGTTT
GCTCTTGGTCCTTATCATATTATTTTGTTACTTCCGGTATAAAAGACAAG
AAACCAGACCTCGATACAGCATTGGGTTAGAACAGGATGAAACTTACATT
CCTCCTGGAGAATCCCTGAGAGACTTAATTGAGCAGTCTCAGAGCTCAGG
AAGTGGATCAGGCCTCCCTCTGCTGGTCCAAAGGACTATAGCTAAGCAGA
TTCAGATGGTGAAACAGATTGGAAAAGGTCGCTATGGGGAAGTTTGGATG
GGAAAGTGGCGTGGCGAAAAGGTAGCTGTGAAAGTGTTCTTCACCACAGA
GGAAGCCAGCTGGTTCAGAGAGACAGAAATATATCAGACAGTGTTGATGA
GGCATGAAAACATTTTGGGTTTCATTGCTGCAGATATCAAAGGGACAGGG
TCCTGGACCCAGTTGTACCTAATCACAGACTATCATGAAAATGGTTCCCT
TTATGATTATCTGAAGTCCACCACCCTAGACGCTAAATCAATGCTGAAGT
TAGCCTACTCTTCTGTCAGTGGCTTATGTCATTTACACACAGAAATCTTT
AGTACTCAAGGCAAACCAGCAATTGCCCATCGAGATCTGAAAAGTAAAAA
CATTCTGGTGAAGAAAAATGGAACTTGCTGTATTGCTGACCTGGGCCTGG
CTGTTAAATTTATTAGTGATACAAATGAAGTTGACATACCACCTAACACT
CGAGTTGGCACCAAACGCTATATGCCTCCAGAAGTGTTGGACGAGAGCTT
GAACAGAAATCACTTCCAGTCTTACATCATGGCTGACATGTATAGTTTTG
GCCTCATCCTTTGGGAGGTTGCTAGGAGATGTGTATCAGGAGGTATAGTG
GAAGAATACCAGCTTCCTTATCATGACCTAGTGCCCAGTGACCCCTCTTA
TGAGGACATGAGGGAGATTGTGTGCATCAAGAAGTTACGCCCCTCATTCC
CAAACCGGTGGAGCAGTGATGAGTGTCTAAGGCAGATGGGAAAACTCATG
ACAGAATGCTGGGCTCACAATCCTGCATCAAGGCTGACAGCCCTGCGGGT
TAAGAAAACACTTGCCAAAATGTCAGAGTCCCAGGACATTAAACTC

A nucleic acid sequence encoding a processed extracellular ALK6 polypeptide is as follows:

(SEQ ID NO: 94)

AACTTCCTTGATAACATGCTTTTGCGAAGTGCAGGAAAATTAAATGTGGG
CACCAAGAAAGAGGATGGTGAGAGTACAGCCCCCACCCCCCGTCCAAAGG
TCTTGCGTTGTAAATGCCACCACCATTGTCCAGAAGACTCAGTCAACAAT
ATTTGCAGCACAGACGGATATTGTTTCACGATGATAGAAGAGGATGACTC
TGGGTTGCCTGTGGTCACTTCTGGTTGCCTAGGACTAGAAGGCTCAGATT
TTCAGTGTCGGGACACTCCCATTCCTCATCAAAGAAGATCAATTGAATGC
TGCACAGAAAGGAACGAATGTAATAAAGACCTACACCCTACACTGCCTCC
ATTGAAAAACAGAGATTTTGTTGATGGACCTATACACCACAGG

In certain embodiments, the disclosure relates to heteromultimers that comprise at least one ALK6 polypeptide, which includes fragments, functional variants, and modified forms thereof. Preferably, ALK6 polypeptides for use in accordance with the disclosure (e.g., heteromultimers comprising an ALK6 polypeptide and uses thereof) are soluble (e.g., an extracellular domain of ALK6). In other preferred embodiments, ALK6 polypeptides for use in accordance with the disclosure bind to and/or inhibit (antagonize) activity (e.g., Smad signaling) of one or more TGF-beta superfamily ligands. In some embodiments, heteromultimers of the disclosure comprise at least one ALK6 polypeptide that is at least 70%, 75%, 80%, 85%, 90%, 91%, 92%, 93%, 94%, 95%, 96%, 97%, 98%, 99%, or 100% identical to the amino acid sequence of SEQ ID NO: 34, 35, 91, or 92. In some embodiments, heteromultimer complexes of the disclosure consist or consist essentially of at least one ALK6 polypeptide that is at least 70%, 75%, 80%, 85%, 90%, 91%, 92%, 93%, 94%, 95%, 96%, 97%, 98%, 99%, or 100% identical to the amino acid sequence of SEQ ID NOs: 34, 35, 91, or 92. In some embodiments, heteromultimers of the disclosure comprise at least one ALK6 polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 14-32 (e.g., amino acid residues 14, 15, 16, 17, 18, 19, 20, 21, 22, 23, 24, 25, 26, 27, 28, 29, 30, 31, or 32) of SEQ ID NO: 34, and ends at any one of amino acids 102-126 (e.g., amino acid residues 102, 103, 104, 105, 106, 107, 108, 109, 110, 111, 112, 113, 114, 115, 116, 117, 118, 119, 120, 121, 122, 123, 124, 125, or 126) of SEQ ID NO: 34. In some embodiments, heteromultimers of the disclosure comprise at least one ALK6 polypeptide that is at least 70%, 75%, 80%, 85%, 90%, 91%, 92%, 93%, 94%, 95%, 96%, 97%, 98%, 99%, or 100% identical to amino acids of 14-102 of SEQ ID NO: 34. In some embodiments, heteromultimers of the disclosure comprise at least one ALK6 polypeptide that is at least 70%, 75%, 80%, 85%, 90%, 91%, 92%, 93%, 94%, 95%, 96%, 97%, 98%, 99%, or 100% identical to amino acids of 14-126 of SEQ ID NO: 34. In some embodiments, heteromultimers of the disclosure comprise at least one ALK6 polypeptide that is at least 70%, 75%, 80%, 85%, 90%, 91%, 92%, 93%, 94%, 95%, 96%, 97%, 98%, 99%, or 100% identical to amino acids of 32-102 of SEQ ID NO: 34. In some embodiments, heteromultimers of the disclosure comprise at least one ALK6 polypeptide that is at least 70%, 75%, 80%, 85%, 90%, 91%, 92%, 93%, 94%, 95%, 96%, 97%, 98%, 99%, or 100% identical to amino acids of 32-126 of SEQ ID NO: 34. In some embodiments, heteromultimers of the disclosure comprise at least one ALK6 polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 26-62 (e.g., amino acid residues 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, or 62) of SEQ ID NO: 91, and ends at any one of amino acids 132-156 (e.g., amino acid residues 132, 133, 134, 135, 136, 137, 138, 139, 140, 141, 142, 143, 144, 145, 146, 147, 148, 149, 150, 151, 152, 153, 154, 155, or 156) of SEQ ID NO: 91. In some embodiments, heteromultimers of the disclosure comprise at least one ALK6 polypeptide that is at least 70%, 75%, 80%, 85%, 90%, 91%, 92%, 93%, 94%, 95%, 96%, 97%, 98%, 99%, or 100% identical to amino acids of 26-132 of SEQ ID NO: 91. In some embodiments, heteromultimers of the disclosure comprise at least one ALK6 polypeptide that is at least 70%, 75%, 80%, 85%, 90%, 91%, 92%, 93%, 94%, 95%, 96%, 97%, 98%, 99%, or 100% identical to amino acids of 26-156 of SEQ ID NO: 91. In some embodiments, heteromultimers of the disclosure comprise at least one ALK6 polypeptide that is at least 70%, 75%, 80%, 85%, 90%, 91%, 92%, 93%, 94%, 95%, 96%, 97%, 98%, 99%, or 100% identical to amino acids of 62-132 of SEQ ID NO: 91. In some embodiments, heteromultimers of the disclosure comprise at least one ALK6 polypeptide that is at least 70%, 75%, 80%, 85%, 90%, 91%, 92%, 93%, 94%, 95%, 96%, 97%, 98%, 99%, or 100% identical to amino acids of 62-156 of SEQ ID NO: 91.

In certain aspects, the present disclosure relates to heteromultimers that comprise an ALK7 polypeptide. As used herein, the term “ALK7” refers to a family of activin receptor-like kinase-7 proteins from any species and variants derived from such ALK7 proteins by mutagenesis or other modification. Reference to ALK7 herein is understood to be a reference to any one of the currently identified forms. Members of the ALK7 family are generally transmembrane proteins, composed of a ligand-binding extracellular domain with a cysteine-rich region, a transmembrane domain, and a cytoplasmic domain with predicted serine/threonine kinase activity.

The term “ALK7 polypeptide” includes polypeptides comprising any naturally occurring polypeptide of an ALK7 family member as well as any variants thereof (including mutants, fragments, fusions, and peptidomimetic forms) that retain a useful activity.

Four naturally occurring isoforms of human ALK7 have been described. The sequence of human ALK7 isoform 1 precursor protein (NCBI Ref Seq NP_660302.2) is as follows:

| (SEQ ID NO: 38) | |
|---|---|
| 1 | MTRALCSALR QALLLLAAAA ELSPGLKCVC LLCDSSNFTC QTEGACWASV |
| | MLTNGKEQVI |
| 61 | KSCVSLPELN AQVFCHSSNN VTKTECCFTD FCNNITLHLP TASPNAPKLG |
| | PMELAIIITV |
| 121 | PVCLLSIAAM LTVWACQGRQ CSYRKKKRPN VEEPLSECNL VNAGKTLKDL |
| | IYDVTASGSG |
| 181 | SGLPLLVQRT IARTIVLQEI VGKGRFGEVW HGRWCGEDVA VKIFSSRDER |
| | SWFREAEIYQ |
| 241 | TVMLRHENIL GFIAADNKDN GTWTQLWLVS EYHEQGSLYD YLNRNIVTVA |
| | GMIKLALSIA |
| 301 | SGLAHLHMEI VGTQGKPAIA HRDIKSKNIL VKKCETCAIA DLGLAVKHDS |
| | ILNTIDIPQN |
| 361 | PKVGTKRYMA PEMLDDTMNV NIFESFKRAD IYSVGLVYWE IARRCSVGGI |
| | VEEYQLPYYD |
| 421 | MVPSDPSIEE MRKVVCDQKF RPSIPNQWQS CEALRVMGRI MRECWYANGA |
| | ARLTALRIKK |
| 481 | TISQLCVKED CKA |

The signal peptide is indicated by a single underline and the extracellular domain is indicated in bold font.

A processed extracellular ALK7 isoform 1 polypeptide sequence is as follows:

(SEQ ID NO: 39)

ELSPGLKCVCLLCDSSNFTCQTEGACWASVMLTNGKEQVIKSCVSLPELN
AQVFCHSSNNVTKTECCFTDFCNNITLHLPTASPNAPKLGPME

A nucleic acid sequence encoding human ALK7 isoform 1 precursor protein is shown below (SEQ ID NO: 40), corresponding to nucleotides 244-1722 of Genbank Reference Sequence NM_145259.2. The signal sequence is underlined and the extracellular domain is indicated in bold font.

(SEQ ID NO: 40)

ATGACCCGGGCGCTCTGCTCAGCGCTCCGCCAGGCTCTCCTGCTGCTCGC
AGCGGCCGCCGAGCTCTCGCCAGGACTGAAGTGTGTATGTCTTTTGTGTG
ATTCTTCAAACTTTACCTGCCAAACAGAAGGAGCATGTTGGGCATCAGTC
ATGCTAACCAATGGAAAAGAGCAGGTGATCAAATCCTGTGTCTCCCTTCC
AGAACTGAATGCTCAAGTCTTCTGTCATAGTTCCAACAATGTTACCAAAA
CCGAATGCTGCTTCACAGATTTTTGCAACAACATAACACTGCACCTTCCA
ACAGCATCACCAAATGCCCCAAAACTTGGACCCATGGAGCTGGCCATCAT
TATTACTGTGCCTGTTTGCCTCCTGTCCATAGCTGCGATGCTGACAGTAT
GGGCATGCCAGGGTCGACAGTGCTCCTACAGGAAGAAAAAGAGACCAAAT
GTGGAGGAACCACTCTCTGAGTGCAATCTGGTAAATGCTGGAAAAACTCT
GAAAGATCTGATTTATGATGTGACCGCCTCTGGATCTGGCTCTGGTCTAC
CTCTGTTGGTTCAAAGGACAATTGCAAGGACGATTGTGCTTCAGGAAATA
GTAGGAAAAGGTAGATTTGGTGAGGTGTGGCATGGAAGATGGTGTGGGGA
AGATGTGGCTGTGAAAATATTCTCCTCCAGAGATGAAAGATCTTGGTTTC
GTGAGGCAGAAATTTACCAGACGGTCATGCTGCGACATGAAAACATCCTT
GGTTTCATTGCTGCTGACAACAAAGATAATGGAACTTGGACTCAACTTTG
GCTGGTATCTGAATATCATGAACAGGGCTCCTTATATGACTATTTGAATA
GAAATATAGTGACCGTGGCTGGAATGATCAAGCTGGCGCTCTCAATTGCT
AGTGGTCTGGCACACCTTCATATGGAGATTGTTGGTACACAAGGTAAACC
TGCTATTGCTCATCGAGACATAAAATCAAAGAATATCTTAGTGAAAAAGT
GTGAAACTTGTGCCATAGCGGACTTAGGGTTGGCTGTGAAGCATGATTCA
ATACTGAACACTATCGACATACCTCAGAATCCTAAAGTGGGAACCAAGAG
GTATATGGCTCCTGAAATGCTTGATGATACAATGAATGTGAATATCTTTG
AGTCCTTCAAACGAGCTGACATCTATTCTGTTGGTCTGGTTTACTGGGAA
ATAGCCCGGAGGTGTTCAGTCGGAGGAATTGTTGAGGAGTACCAATTGCC
TTATTATGACATGGTGCCTTCAGATCCCTCGATAGAGGAAATGAGAAAGG
TTGTTTGTGACCAGAAGTTTCGACCAAGTATCCCAAACCAGTGGCAAAGT
TGTGAAGCACTCCGAGTCATGGGGAGAATAATGCGTGAGTGTTGGTATGC
CAACGGAGCGGCCCGCCTAACTGCTCTTCGTATTAAGAAGACTATATCTC
AACTTTGTGTCAAAGAAGACTGCAAAGCC

A nucleic acid sequence encoding a processed extracellular ALK7 polypeptide (isoform 1) is as follows:

(SEQ ID NO: 41)

GAGCTCTCGCCAGGACTGAAGTGTGTATGTCTTTTGTGTGATTCTTCAAA
CTTTACCTGCCAAACAGAAGGAGCATGTTGGGCATCAGTCATGCTAACCA
ATGGAAAAGAGCAGGTGATCAAATCCTGTGTCTCCCTTCCAGAACTGAAT
GCTCAAGTCTTCTGTCATAGTTCCAACAATGTTACCAAAACCGAATGCTG
CTTCACAGATTTTTGCAACAACATAACACTGCACCTTCCAACAGCATCAC
CAAATGCCCCAAAACTTGGACCCATGGAG

The amino acid sequence of an alternative isoform of human ALK7, isoform 2 (NCBI Ref Seq NP_001104501.1), is shown in its processed form as follows (SEQ ID NO: 301), where the extracellular domain is indicated in bold font.

| (SEQ ID NO: 301) | |
|---|---|
| 1 | MLTNGKEQVI KSCVSLPELN AQVFCHSSNN VTKTECCFTD FCNNITLHLP |
| | TASPNAPKLG |
| 61 | PMELAIIITV PVCLLSIAAM LTVWACQGRQ CSYRKKKRPN VEEPLSECNL |
| | VNAGKTLKDL |
| 121 | IYDVTASGSG SGLPLLVQRT IARTIVLQEI VGKGRFGEVW HGRWCGEDVA |
| | VKIFSSRDER |
| 181 | SWFREAEIYQ TVMLRHENIL GFIAADNKDN GTWTQLWLVS EYHEQGSLYD |
| | YLNRNIVTVA |
| 241 | GMIKLALSIA SGLAHLHMEI VGTQGKPAIA HRDIKSKNIL VKKCETCAIA |
| | DLGLAVKHDS |
| 301 | ILNTIDIPQN PKVGTKRYMA PEMLDDTMNV NIFESFKRAD IYSVGLVYWE |
| | IARRCSVGGI |
| 361 | VEEYQLPYYD MVPSDPSIEE MRKVVCDQKF RPSIPNQWQS CEALRVMGRI |
| | MRECWYANGA |
| 421 | ARLTALRIKK TISQLCVKED CKA |

An amino acid sequence of an extracellular ALK7 polypeptide (isoform 2) is as follows:

(SEQ ID NO: 302)

MLTNGKEQVIKSCVSLPELNAQVFCHSSNNVTKTECCFTDFCNNITLHLP
TASPNAPKLGPME.

A nucleic acid sequence encoding the processed ALK7 polypeptide (isoform 2) is shown below (SEQ ID NO: 303), corresponding to nucleotides 279-1607 of NCBI Reference Sequence NM_001111031.1. The extracellular domain is indicated in bold font.

(SEQ ID NO: 303)

ATGCTAACCAATGGAAAAGAGCAGGTGATCAAATCCTGTGTCTCCCTTCC
AGAACTGAATGCTCAAGTCTTCTGTCATAGTTCCAACAATGTTACCAAAA
CCGAATGCTGCTTCACAGATTTTTGCAACAACATAACACTGCACCTTCCA
ACAGCATCACCAAATGCCCCAAAACTTGGACCCATGGAGCTGGCCATCAT
TATTACTGTGCCTGTTTGCCTCCTGTCCATAGCTGCGATGCTGACAGTAT
GGGCATGCCAGGGTCGACAGTGCTCCTACAGGAAGAAAAAGAGACCAAAT
GTGGAGGAACCACTCTCTGAGTGCAATCTGGTAAATGCTGGAAAAACTCT
GAAAGATCTGATTTATGATGTGACCGCCTCTGGATCTGGCTCTGGTCTAC
CTCTGTTGGTTCAAAGGACAATTGCAAGGACGATTGTGCTTCAGGAAATA
GTAGGAAAAGGTAGATTTGGTGAGGTGTGGCATGGAAGATGGTGTGGGGA
AGATGTGGCTGTGAAAATATTCTCCTCCAGAGATGAAAGATCTTGGTTTC
GTGAGGCAGAAATTTACCAGACGGTCATGCTGCGACATGAAAACATCCTT
GGTTTCATTGCTGCTGACAACAAAGATAATGGAACTTGGACTCAACTTTG
GCTGGTATCTGAATATCATGAACAGGGCTCCTTATATGACTATTTGAATA
GAAATATAGTGACCGTGGCTGGAATGATCAAGCTGGCGCTCTCAATTGCT
AGTGGTCTGGCACACCTTCATATGGAGATTGTTGGTACACAAGGTAAACC
TGCTATTGCTCATCGAGACATAAAATCAAAGAATATCTTAGTGAAAAAGT
GTGAAACTTGTGCCATAGCGGACTTAGGGTTGGCTGTGAAGCATGATTCA
ATACTGAACACTATCGACATACCTCAGAATCCTAAAGTGGGAACCAAGAG
GTATATGGCTCCTGAAATGCTTGATGATACAATGAATGTGAATATCTTTG
AGTCCTTCAAACGAGCTGACATCTATTCTGTTGGTCTGGTTTACTGGGAA
ATAGCCCGGAGGTGTTCAGTCGGAGGAATTGTTGAGGAGTACCAATTGCC
TTATTATGACATGGTGCCTTCAGATCCCTCGATAGAGGAAATGAGAAAGG
TTGTTTGTGACCAGAAGTTTCGACCAAGTATCCCAAACCAGTGGCAAAGT
TGTGAAGCACTCCGAGTCATGGGGAGAATAATGCGTGAGTGTTGGTATGC
CAACGGAGCGGCCCGCCTAACTGCTCTTCGTATTAAGAAGACTATATCTC
AACTTTGTGTCAAAGAAGACTGCAAAGCC

A nucleic acid sequence encoding an extracellular ALK7 polypeptide (isoform 2) is as follows (SEQ ID NO: 304):

(SEQ ID NO: 304)

ATGCTAACCAATGGAAAAGAGCAGGTGATCAAATCCTGTGTCTCCCTTCC
AGAACTGAATGCTCAAGTCTTCTGTCATAGTTCCAACAATGTTACCAAAA
CCGAATGCTGCTTCACAGATTTTTGCAACAACATAACACTGCACCTTCCA
ACAGCATCACCAAATGCCCCAAAACTTGGACCCATGGAG

An amino acid sequence of an alternative human ALK7 precursor protein, isoform 3 (NCBI Ref Seq NP_001104502.1), is shown as follows (SEQ ID NO: 305), where the signal peptide is indicated by a single underline.

| (SEQ ID NO: 305) | |
|---|---|
| 1 | MTRALCSALR QALLLLAAAA ELSPGLKCVC LLCDSSNFTC QTEGACWASV |
| | MLTNGKEQVI |
| 61 | KSCVSLPELN AQVFCHSSNN VTKTECCFTD FCNNITLHLP TGLPLLVQRT |
| | IARTIVLQEI |
| 121 | VGKGRFGEVW HGRWCGEDVA VKIFSSRDER SWFREAEIYQ TVMLRHENIL |
| | GFIAADNKDN |
| 181 | GTWTQLWLVS EYHEQGSLYD YLNRNIVTVA GMIKLALSIA SGLAHLHMEI |
| | VGTQGKPAIA |
| 241 | HRDIKSKNIL VKKCETCAIA DLGLAVKHDS ILNTIDIPQN PKVGTKRYMA |
| | PEMLDDTMNV |
| 301 | NIFESFKRAD IYSVGLVYWE IARRCSVGGI VEEYQLPYYD MVPSDPSIEE |
| | MRKVVCDQKF |
| 361 | RPSIPNQWQS CEALRVMGRI MRECWYANGA ARLTALRIKK TISQLCVKED CKA |

An amino acid sequence of an processed ALK7 polypeptide (isoform 3) is as follows (SEQ ID NO: 306). This isoform lacks a transmembrane domain and is therefore proposed to be soluble in its entirety (Roberts et al., 2003, Biol Reprod 68:1719-1726). N-terminal variants of SEQ ID NO: 306 are predicted as described below.

| (SEQ ID NO: 306) | |
|---|---|
| 1 | ELSPGLKCVC LLCDSSNFTC QTEGACWASV MLTNGKEQVI KSCVSLPELN |
| | AQVFCHSSNN |
| 61 | VTKTECCFTD FCNNITLHLP TGLPLLVQRT IARTIVLQEI VGKGRFGEVW |
| | HGRWCGEDVA |
| 121 | VKIFSSRDER SWFREAEIYQ TVMLRHENIL GFIAADNKDN GTWTQLWLVS |
| | EYHEQGSLYD |
| 181 | YLNRNIVTVA GMIKLALSIA SGLAHLHMEI VGTQGKPAIA HRDIKSKNIL |
| | VKKCETCAIA |
| 241 | DLGLAVKHDS ILNTIDIPQN PKVGTKRYMA PEMLDDTMNV NIFESFKRAD |
| | IYSVGLVYWE |
| 301 | IARRCSVGGI VEEYQLPYYD MVPSDPSIEE MRKVVCDQKF RPSIPNQWQS |
| | CEALRVMGRI |
| 361 | MRECWYANGA ARLTALRIKK TISQLCVKED CKA |

A nucleic acid sequence encoding the unprocessed ALK7 polypeptide precursor protein (isoform 3) is shown below (SEQ ID NO: 307), corresponding to nucleotides 244-1482 of NCBI Reference Sequence NM_001111032.1. The signal sequence is indicated by solid underline.

(SEQ ID NO: 307)

ATGACCCGGGCGCTCTGCTCAGCGCTCCGCCAGGCTCTCCTGCTGCTCGC
AGCGGCCGCCGAGCTCTCGCCAGGACTGAAGTGTGTATGTCTTTTGTGTG
ATTCTTCAAACTTTACCTGCCAAACAGAAGGAGCATGTTGGGCATCAGTC
ATGCTAACCAATGGAAAAGAGCAGGTGATCAAATCCTGTGTCTCCCTTCC
AGAACTGAATGCTCAAGTCTTCTGTCATAGTTCCAACAATGTTACCAAAA
CCGAATGCTGCTTCACAGATTTTTGCAACAACATAACACTGCACCTTCCA
ACAGGTCTACCTCTGTTGGTTCAAAGGACAATTGCAAGGACGATTGTGCT
TCAGGAAATAGTAGGAAAAGGTAGATTTGGTGAGGTGTGGCATGGAAGAT
GGTGTGGGGAAGATGTGGCTGTGAAAATATTCTCCTCCAGAGATGAAAGA
TCTTGGTTTCGTGAGGCAGAAATTTACCAGACGGTCATGCTGCGACATGA
AAACATCCTTGGTTTCATTGCTGCTGACAACAAAGATAATGGAACTTGGA
CTCAACTTTGGCTGGTATCTGAATATCATGAACAGGGCTCCTTATATGAC
TATTTGAATAGAAATATAGTGACCGTGGCTGGAATGATCAAGCTGGCGCT
CTCAATTGCTAGTGGTCTGGCACACCTTCATATGGAGATTGTTGGTACAC
AAGGTAAACCTGCTATTGCTCATCGAGACATAAAATCAAAGAATATCTTA
GTGAAAAAGTGTGAAACTTGTGCCATAGCGGACTTAGGGTTGGCTGTGAA
GCATGATTCAATACTGAACACTATCGACATACCTCAGAATCCTAAAGTGG
GAACCAAGAGGTATATGGCTCCTGAAATGCTTGATGATACAATGAATGTG
AATATCTTTGAGTCCTTCAAACGAGCTGACATCTATTCTGTTGGTCTGGT
TTACTGGGAAATAGCCCGGAGGTGTTCAGTCGGAGGAATTGTTGAGGAGT
ACCAATTGCCTTATTATGACATGGTGCCTTCAGATCCCTCGATAGAGGAA
ATGAGAAAGGTTGTTTGTGACCAGAAGTTTCGACCAAGTATCCCAAACCA
GTGGCAAAGTTGTGAAGCACTCCGAGTCATGGGGAGAATAATGCGTGAGT
GTTGGTATGCCAACGGAGCGGCCCGCCTAACTGCTCTTCGTATTAAGAAG
ACTATATCTCAACTTTGTGTCAAAGAAGACTGCAAAGCC

A nucleic acid sequence encoding a processed ALK7 polypeptide (isoform 3) is as follows (SEQ ID NO: 308):

(SEQ ID NO: 308)

GAGCTCTCGCCAGGACTGAAGTGTGTATGTCTTTTGTGTGATTCTTCAAA
CTTTACCTGCCAAACAGAAGGAGCATGTTGGGCATCAGTCATGCTAACCA
ATGGAAAAGAGCAGGTGATCAAATCCTGTGTCTCCCTTCCAGAACTGAAT
GCTCAAGTCTTCTGTCATAGTTCCAACAATGTTACCAAAACCGAATGCTG
CTTCACAGATTTTTGCAACAACATAACACTGCACCTTCCAACAGGTCTAC
CTCTGTTGGTTCAAAGGACAATTGCAAGGACGATTGTGCTTCAGGAAATA
GTAGGAAAAGGTAGATTTGGTGAGGTGTGGCATGGAAGATGGTGTGGGGA
AGATGTGGCTGTGAAAATATTCTCCTCCAGAGATGAAAGATCTTGGTTTC
GTGAGGCAGAAATTTACCAGACGGTCATGCTGCGACATGAAAACATCCTT
GGTTTCATTGCTGCTGACAACAAAGATAATGGAACTTGGACTCAACTTTG
GCTGGTATCTGAATATCATGAACAGGGCTCCTTATATGACTATTTGAATA
GAAATATAGTGACCGTGGCTGGAATGATCAAGCTGGCGCTCTCAATTGCT
AGTGGTCTGGCACACCTTCATATGGAGATTGTTGGTACACAAGGTAAACC
TGCTATTGCTCATCGAGACATAAAATCAAAGAATATCTTAGTGAAAAAGT
GTGAAACTTGTGCCATAGCGGACTTAGGGTTGGCTGTGAAGCATGATTCA
ATACTGAACACTATCGACATACCTCAGAATCCTAAAGTGGGAACCAAGAG
GTATATGGCTCCTGAAATGCTTGATGATACAATGAATGTGAATATCTTTG
AGTCCTTCAAACGAGCTGACATCTATTCTGTTGGTCTGGTTTACTGGGAA
ATAGCCCGGAGGTGTTCAGTCGGAGGAATTGTTGAGGAGTACCAATTGCC
TTATTATGACATGGTGCCTTCAGATCCCTCGATAGAGGAAATGAGAAAGG
TTGTTTGTGACCAGAAGTTTCGACCAAGTATCCCAAACCAGTGGCAAAGT
TGTGAAGCACTCCGAGTCATGGGGAGAATAATGCGTGAGTGTTGGTATGC
CAACGGAGCGGCCCGCCTAACTGCTCTTCGTATTAAGAAGACTATATCTC
AACTTTGTGTCAAAGAAGACTGCAAAGCC

An amino acid sequence of an alternative human ALK7 precursor protein, isoform 4 (NCBI Ref Seq NP_001104503.1), is shown as follows (SEQ ID NO: 309), where the signal peptide is indicated by a single underline.

| (SEQ ID NO: 309) | |
|---|---|
| 1 | MTRALCSALR QALLLLAAAA ELSPGLKCVC LLCDSSNFTC QTEGACWASV |
| | MLTNGKEQVI |
| 61 | KSCVSLPELN AQVFCHSSNN VTKTECCFTD FCNNITLHLP TDNGTWTQLW |
| | LVSEYHEQGS |
| 121 | LYDYLNRNIV TVAGMIKLAL SIASGLAHLH MEIVGTQGKP AIAHRDIKSK |
| | NILVKKCETC |
| 181 | AIADLGLAVK HDSILNTIDI PQNPKVGTKR YMAPEMLDDT MNVNIFESFK |
| | RADIYSVGLV |
| 241 | YWEIARRCSV GGIVEEYQLP YYDMVPSDPS IEEMRKVVCD QKFRPSIPNQ |
| | WQSCEALRVM |
| 301 | GRIMRECWYA NGAARLTALR IKKTISQLCV KEDCKA |

An amino acid sequence of a processed ALK7 polypeptide (isoform 4) is as follows (SEQ ID NO: 310). Like ALK7 isoform 3, isoform 4 lacks a transmembrane domain and is therefore proposed to be soluble in its entirety (Roberts et al., 2003, Biol Reprod 68:1719-1726). N-terminal variants of SEQ ID NO: 310 are predicted as described below.

| (SEQ ID NO: 310) | |
|---|---|
| 1 | ELSPGLKCVC LLCDSSNFTC QTEGACWASV MLTNGKEQVI KSCVSLPELN |
| | AQVFCHSSNN |
| 61 | VTKTECCFTD FCNNITLHLP TDNGTWTQLW LVSEYHEQGS LYDYLNRNIV |
| | TVAGMIKLAL |
| 121 | SIASGLAHLH MEIVGTQGKP AIAHRDIKSK NILVKKCETC AIADLGLAVK |
| | HDSILNTIDI |
| 181 | PQNPKVGTKR YMAPEMLDDT MNVNIFESFK RADIYSVGLV YWEIARRCSV |
| | GGIVEEYQLP |
| 240 | YYDMVPSDPS IEEMRKVVCD QKFRPSIPNQ WQSCEALRVM GRIMRECWYA |
| | NGAARLTALR |
| 301 | IKKTISQLCV KEDCKA |

A nucleic acid sequence encoding an unprocessed ALK7 polypeptide precursor protein (isoform 4) is shown below (SEQ ID NO: 311), corresponding to nucleotides 244-1244 of NCBI Reference Sequence NM_001111033.1. The signal sequence is indicated by solid underline.

(SEQ ID NO: 311)

ATGACCCGGGCGCTCTGCTCAGCGCTCCGCCAGGCTCTCCTGCTGCTCGC
AGCGGCCGCCGAGCTCTCGCCAGGACTGAAGTGTGTATGTCTTTTGTGTG
ATTCTTCAAACTTTACCTGCCAAACAGAAGGAGCATGTTGGGCATCAGTC
ATGCTAACCAATGGAAAAGAGCAGGTGATCAAATCCTGTGTCTCCCTTCC
AGAACTGAATGCTCAAGTCTTCTGTCATAGTTCCAACAATGTTACCAAAA
CCGAATGCTGCTTCACAGATTTTTGCAACAACATAACACTGCACCTTCCA
ACAGATAATGGAACTTGGACTCAACTTTGGCTGGTATCTGAATATCATGA
ACAGGGCTCCTTATATGACTATTTGAATAGAAATATAGTGACCGTGGCTG
GAATGATCAAGCTGGCGCTCTCAATTGCTAGTGGTCTGGCACACCTTCAT
ATGGAGATTGTTGGTACACAAGGTAAACCTGCTATTGCTCATCGAGACAT
AAAATCAAAGAATATCTTAGTGAAAAAGTGTGAAACTTGTGCCATAGCGG
ACTTAGGGTTGGCTGTGAAGCATGATTCAATACTGAACACTATCGACATA
CCTCAGAATCCTAAAGTGGGAACCAAGAGGTATATGGCTCCTGAAATGCT
TGATGATACAATGAATGTGAATATCTTTGAGTCCTTCAAACGAGCTGACA
TCTATTCTGTTGGTCTGGTTTACTGGGAAATAGCCCGGAGGTGTTCAGTC
GGAGGAATTGTTGAGGAGTACCAATTGCCTTATTATGACATGGTGCCTTC
AGATCCCTCGATAGAGGAAATGAGAAAGGTTGTTTGTGACCAGAAGTTTC
GACCAAGTATCCCAAACCAGTGGCAAAGTTGTGAAGCACTCCGAGTCATG
GGGAGAATAATGCGTGAGTGTTGGTATGCCAACGGAGCGGCCCGCCTAAC
TGCTCTTCGTATTAAGAAGACTATATCTCAACTTTGTGTCAAAGAAGACT
GCAAAGCCTAA

A nucleic acid sequence encoding a processed ALK7 polypeptide (isoform 4) is as follows (SEQ ID NO: 312):

(SEQ ID NO: 312)

GAGCTCTCGCCAGGACTGAAGTGTGTATGTCTTTTGTGTGATTCTTCAAA
CTTTACCTGCCAAACAGAAGGAGCATGTTGGGCATCAGTCATGCTAACCA
ATGGAAAAGAGCAGGTGATCAAATCCTGTGTCTCCCTTCCAGAACTGAAT
GCTCAAGTCTTCTGTCATAGTTCCAACAATGTTACCAAAACCGAATGCTG
CTTCACAGATTTTTGCAACAACATAACACTGCACCTTCCAACAGATAATG
GAACTTGGACTCAACTTTGGCTGGTATCTGAATATCATGAACAGGGCTCC
TTATATGACTATTTGAATAGAAATATAGTGACCGTGGCTGGAATGATCAA
GCTGGCGCTCTCAATTGCTAGTGGTCTGGCACACCTTCATATGGAGATTG
TTGGTACACAAGGTAAACCTGCTATTGCTCATCGAGACATAAAATCAAAG
AATATCTTAGTGAAAAAGTGTGAAACTTGTGCCATAGCGGACTTAGGGTT
GGCTGTGAAGCATGATTCAATACTGAACACTATCGACATACCTCAGAATC
CTAAAGTGGGAACCAAGAGGTATATGGCTCCTGAAATGCTTGATGATACA
ATGAATGTGAATATCTTTGAGTCCTTCAAACGAGCTGACATCTATTCTGT
TGGTCTGGTTTACTGGGAAATAGCCCGGAGGTGTTCAGTCGGAGGAATTG
TTGAGGAGTACCAATTGCCTTATTATGACATGGTGCCTTCAGATCCCTCG
ATAGAGGAAATGAGAAAGGTTGTTTGTGACCAGAAGTTTCGACCAAGTAT
CCCAAACCAGTGGCAAAGTTGTGAAGCACTCCGAGTCATGGGGAGAATAA
TGCGTGAGTGTTGGTATGCCAACGGAGCGGCCCGCCTAACTGCTCTTCGT
ATTAAGAAGACTATATCTCAACTTTGTGTCAAAGAAGACTGCAAAGCCTA
A

Based on the signal sequence of full-length ALK7 (isoform 1) in the rat (see NCBI Reference Sequence NP_620790.1) and on the high degree of sequence identity between human and rat ALK7, it is predicted that a processed form of human ALK7 isoform 1 is as follows (SEQ ID NO: 313).

| (SEQ ID NO: 313) | |
|---|---|
| 1 | LKCVCLLCDS SNFTCQTEGA CWASVMLTNG KEQVIKSCVS LPELNAQVFC |
| | HSSNNVTKTE |
| 61 | CCFTDFCNNI TLHLPTASPN APKLGPME |

Active variants of processed ALK7 isoform 1 are predicted in which SEQ ID NO: 39 is truncated by 1, 2, 3, 4, 5, 6, or 7 amino acids at the N-terminus and SEQ ID NO: 313 is truncated by 1 or 2 amino acids at the N-terminus. Consistent with SEQ ID NO: 313, it is further expected that leucine is the N-terminal amino acid in the processed forms of human ALK7 isoform 3 (SEQ ID NO: 306) and human ALK7 isoform 4 (SEQ ID NO: 310). In certain embodiments, the disclosure relates to heteromultimers that comprise at least one ALK7 polypeptide, which includes fragments, functional variants, and modified forms thereof. Preferably, ALK7 polypeptides for use in accordance with inventions of the disclosure (e.g., heteromultimers comprising an ALK7 polypeptide and uses thereof) are soluble (e.g., an extracellular domain of ALK7). In other preferred embodiments, ALK7 polypeptides for use in accordance with the inventions of the disclosure bind to and/or inhibit (antagonize) activity (e.g., Smad signaling) of one or more TGF-beta superfamily ligands. In some embodiments, heteromultimers of the disclosure comprise at least one ALK7 polypeptide that is at least 70%, 70%, 75%, 80%, 85%, 90%, 91%, 92%, 93%, 94%, 95%, 96%, 97%, 98%, 99%, or 100% identical to the amino acid sequence of SEQ ID NO: 38, 39, 301, 302, 305, 306, 309, 310, or 313. In some embodiments, heteromultimers of the disclosure comprise at least one ALK7 polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 21-28 (e.g., amino acid residues 21, 22, 23, 24, 25, 26, 27, or 28) of SEQ ID NO: 38, and ends at any one of amino acids 92-113 (e.g., amino acid residues 92, 93, 94, 95, 96, 97, 98, 99, 100, 101, 102, 103, 104, 105, 106, 107, 108, 109, 110, 111, 112, or 113) of SEQ ID NO: 38. In some embodiments, heteromultimers of the disclosure comprise at least one ALK7 polypeptide that is at least 70%, 75%, 80%, 85%, 90%, 91%, 92%, 93%, 94%, 95%, 96%, 97%, 98%, 99%, or 100% identical to amino acids of 21-92 of SEQ ID NO: 38. In some embodiments, heteromultimers of the disclosure comprise at least one ALK7 polypeptide that is at least 70%, 75%, 80%, 85%, 90%, 91%, 92%, 93%, 94%, 95%, 96%, 97%, 98%, 99%, or 100% identical to amino acids of 21-113 of SEQ ID NO: 38. In some embodiments, heteromultimers of the disclosure comprise at least one ALK7 polypeptide that is at least 70%, 75%, 80%, 85%, 90%, 91%, 92%, 93%, 94%, 95%, 96%, 97%, 98%, 99%, or 100% identical to amino acids of 28-92 of SEQ ID NO: 38. In some embodiments, heteromultimers of the disclosure comprise at least one ALK7 polypeptide that is at least 70%, 75%, 80%, 85%, 90%, 91%, 92%, 93%, 94%, 95%, 96%, 97%, 98%, 99%, or 100% identical to amino acids of 28-113 of SEQ ID NO: 38. In some embodiments, heteromultimers of the disclosure comprise at least one ALK7 polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 1-13 (e.g., amino acid residues 1, 2, 3, 4, 5, 6, 7, 8, 9, 10, 11, 12, or 13) of SEQ ID NO: 301, and ends at any one of amino acids 42-63 (e.g., amino acid residues 42, 43, 44, 45, 46, 47, 48, 49, 50, 51, 52, 53, 54, 55, 56, 57, 58, 59, 60, 61, 62, or 63) of SEQ ID NO: 301. In some embodiments, heteromultimers of the disclosure comprise at least one ALK7 polypeptide that is at least 70%, 75%, 80%, 85%, 90%, 91%, 92%, 93%, 94%, 95%, 96%, 97%, 98%, 99%, or 100% identical to amino acids of 1-42 of SEQ ID NO: 301. In some embodiments, heteromultimers of the disclosure comprise at least one ALK7 polypeptide that is at least 70%, 75%, 80%, 85%, 90%, 91%, 92%, 93%, 94%, 95%, 96%, 97%, 98%, 99%, or 100% identical to amino acids of 1-63 of SEQ ID NO: 301. In some embodiments, heteromultimers of the disclosure comprise at least one ALK7 polypeptide that is at least 70%, 75%, 80%, 85%, 90%, 91%, 92%, 93%, 94%, 95%, 96%, 97%, 98%, 99%, or 100% identical to amino acids of 13-42 of SEQ ID NO: 301. In some embodiments, heteromultimers of the disclosure comprise at least one ALK7 polypeptide that is at least 70%, 75%, 80%, 85%, 90%, 91%, 92%, 93%, 94%, 95%, 96%, 97%, 98%, 99%, or 100% identical to amino acids of 13-63 of SEQ ID NO: 301. In some embodiments, heteromultimers of the disclosure comprise at least one ALK7 polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 21-28 (e.g., amino acid residues 21, 22, 23, 24, 25, 26, 27, or 28) of SEQ ID NO: 305, and ends at any one of amino acids 411-413 (e.g., amino acid residues 411, 412, or 413) of SEQ ID NO: 305. In some embodiments, heteromultimers of the disclosure comprise at least one ALK7 polypeptide that is at least 70%, 75%, 80%, 85%, 90%, 91%, 92%, 93%, 94%, 95%, 96%, 97%, 98%, 99%, or 100% identical to amino acids of 21-411 of SEQ ID NO: 305. In some embodiments, heteromultimers of the disclosure comprise at least one ALK7 polypeptide that is at least 70%, 75%, 80%, 85%, 90%, 91%, 92%, 93%, 94%, 95%, 96%, 97%, 98%, 99%, or 100% identical to amino acids of 21-413 of SEQ ID NO: 305. In some embodiments, heteromultimers of the disclosure comprise at least one ALK7 polypeptide that is at least 70%, 75%, 80%, 85%, 90%, 91%, 92%, 93%, 94%, 95%, 96%, 97%, 98%, 99%, or 100% identical to amino acids of 28-411 of SEQ ID NO: 305. In some embodiments, heteromultimers of the disclosure comprise at least one ALK7 polypeptide that is at least 70%, 75%, 80%, 85%, 90%, 91%, 92%, 93%, 94%, 95%, 96%, 97%, 98%, 99%, or 100% identical to amino acids of 28-413 of SEQ ID NO: 305. In some embodiments, heteromultimers of the disclosure comprise at least one ALK7 polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 21-28 (e.g., amino acid residues 21, 22, 23, 24, 25, 26, 27, or 28) of SEQ ID NO: 309, and ends at any one of amino acids 334-336 (e.g., amino acid residues 334, 335, or 336) of SEQ ID NO: 309. In some embodiments, heteromultimers of the disclosure comprise at least one ALK7 polypeptide that is at least 70%, 75%, 80%, 85%, 90%, 91%, 92%, 93%, 94%, 95%, 96%, 97%, 98%, 99%, or 100% identical to amino acids of 21-334 of SEQ ID NO: 309. In some embodiments, heteromultimers of the disclosure comprise at least one ALK7 polypeptide that is at least 70%, 75%, 80%, 85%, 90%, 91%, 92%, 93%, 94%, 95%, 96%, 97%, 98%, 99%, or 100% identical to amino acids of 21-336 of SEQ ID NO: 309. In some embodiments, heteromultimers of the disclosure comprise at least one ALK7 polypeptide that is at least 70%, 75%, 80%, 85%, 90%, 91%, 92%, 93%, 94%, 95%, 96%, 97%, 98%, 99%, or 100% identical to amino acids of 28-334 of SEQ ID NO: 309. In some embodiments, heteromultimers of the disclosure comprise at least one ALK7 polypeptide that is at least 70%, 75%, 80%, 85%, 90%, 91%, 92%, 93%, 94%, 95%, 96%, 97%, 98%, 99%, or 100% identical to amino acids of 28-336 of SEQ ID NO: 309.

The term “endoglin polypeptide” includes polypeptides comprising any naturally occurring endoglin protein (encoded by ENG or one of its nonhuman orthologs) as well as any variants thereof (including mutants, fragments, fusions, and peptidomimetic forms) that retain a useful activity.

The human endoglin isoform 1 precursor protein sequence (NCBI Ref Seq NP_001108225.1) is as follows:

| (SEQ ID NO: 501) | |
|---|---|
| 1 | MDRGTLPLAV ALLLASCSLS PTSLAETVHC DLQPVGPERG EVTYTTSQVS KGCVAQAPNA |
| 61 | ILEVHVLFLE FPTGPSQLEL TLQASKQNGT WPREVLLVLS VNSSVFLHLQ ALGIPLHLAY |
| 121 | NSSLVTFQEP PGVNTTELPS FPKTQILEWA AERGPITSAA ELNDPQSILL RLGQAQGSLS |
| 181 | FCMLEASQDM GRTLEWRPRT PALVRGCHLE GVAGHKEAHI LRVLPGHSAG PRTVTVKVEL |
| 241 | SCAPGDLDAV LILQGPPYVS WLIDANHNMQ IWTTGEYSFK IFPEKNIRGF KLPDTPQGLL |
| 301 | GEARMLNASI VASFVELPLA SIVSLHASSC GGRLQTSPAP IQTTPPKDTC SPELLMSLIQ |
| 361 | TKCADDAMTL VLKKELVAHL KCTITGLTFW DPSCEAEDRG DKFVLRSAYS SCGMQVSASM |
| 421 | ISNEAVVNIL SSSSPQRKKV HCLNMDSLSF QLGLYLSPHF LQASNTIEPG QQSFVQVRVS |
| 481 | PSVSEFLLQL DSCHLDLGPE GGTVELIQGR AAKGNCVSLL SPSPEGDPRF SFLLHFYTVP |
| 541 | 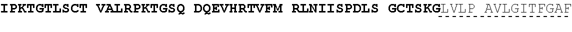 |
| 601 | 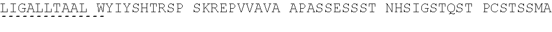 |

The signal peptide is indicated by single underline, the extracellular domain is indicated in bold font, and the transmembrane domain is indicated by dotted underline.

A processed extracellular endoglin polypeptide sequence (isoform 1) is as follows:

(SEQ ID NO: 502)

ETVHCDLQPVGPERGEVTYTTSQVSKGCVAQAPNAILEVHVLFLEFPTGP
SQLELTLQASKQNGTWPREVLLVLSVNSSVFLHLQALGIPLHLAYNSSLV
TFQEPPGVNTTELPSFPKTQILEWAAERGPITSAAELNDPQSILLRLGQA
QGSLSFCMLEASQDMGRTLEWRPRTPALVRGCHLEGVAGHKEAHILRVLP
GHSAGPRTVTVKVELSCAPGDLDAVLILQGPPYVSWLIDANHNMQIWTTG
EYSFKIFPEKNIRGFKLPDTPQGLLGEARMLNASIVASFVELPLASIVSL
HASSCGGRLQTSPAPIQTTPPKDTCSPELLMSLIQTKCADDAMTLVLKKE
LVAHLKCTITGLTFWDPSCEAEDRGDKFVLRSAYSSCGMQVSASMISNEA
VVNILSSSSPQRKKVHCLNMDSLSFQLGLYLSPHFLQASNTIEPGQQSFV
QVRVSPSVSEFLLQLDSCHLDLGPEGGTVELIQGRAAKGNCVSLLSPSPE
GDPRFSFLLHFYTVPIPKTGTLSCTVALRPKTGSQDQEVHRTVFMRLNII
SPDLSGCTSKG

A nucleic acid sequence encoding unprocessed human ENG isoform 1 precursor protein is shown below (SEQ ID NO: 503), corresponding to nucleotides 419-2392 of NCBI Reference Sequence NM_001114753.2. The signal sequence is underlined.

| (SEQ ID NO: 503) | |
|---|---|
| 1 | ATGGACCGCG GCACGCTCCC TCTGGCTGTT GCCCTGCTGC TGGCCAGCTG |
| 51 | CAGCCTCAGC CCCACAAGTC TTGCAGAAAC AGTCCATTGT GACCTTCAGC |
| 101 | CTGTGGGCCC CGAGAGGGGC GAGGTGACAT ATACCACTAG CCAGGTCTCG |
| 151 | AAGGGCTGCG TGGCTCAGGC CCCCAATGCC ATCCTTGAAG TCCATGTCCT |
| 201 | CTTCCTGGAG TTCCCAACGG GCCCGTCACA GCTGGAGCTG ACTCTCCAGG |
| 251 | CATCCAAGCA AAATGGCACC TGGCCCCGAG AGGTGCTTCT GGTCCTCAGT |
| 301 | GTAAACAGCA GTGTCTTCCT GCATCTCCAG GCCCTGGGAA TCCCACTGCA |
| 351 | CTTGGCCTAC AATTCCAGCC TGGTCACCTT CCAAGAGCCC CCGGGGGTCA |
| 401 | ACACCACAGA GCTGCCATCC TTCCCCAAGA CCCAGATCCT TGAGTGGGCA |
| 451 | GCTGAGAGGG GCCCCATCAC CTCTGCTGCT GAGCTGAATG ACCCCCAGAG |
| 501 | CATCCTCCTC CGACTGGGCC AAGCCCAGGG GTCACTGTCC TTCTGCATGC |
| 551 | TGGAAGCCAG CCAGGACATG GGCCGCACGC TCGAGTGGCG GCCGCGTACT |
| 601 | CCAGCCTTGG TCCGGGGCTG CCACTTGGAA GGCGTGGCCG GCCACAAGGA |
| 651 | GGCGCACATC CTGAGGGTCC TGCCGGGCCA CTCGGCCGGG CCCCGGACGG |
| 701 | TGACGGTGAA GGTGGAACTG AGCTGCGCAC CCGGGGATCT CGATGCCGTC |
| 751 | CTCATCCTGC AGGGTCCCCC CTACGTGTCC TGGCTCATCG ACGCCAACCA |
| 801 | CAACATGCAG ATCTGGACCA CTGGAGAATA CTCCTTCAAG ATCTTTCCAG |
| 851 | AGAAAAACAT TCGTGGCTTC AAGCTCCCAG ACACACCTCA AGGCCTCCTG |
| 901 | GGGGAGGCCC GGATGCTCAA TGCCAGCATT GTGGCATCCT TCGTGGAGCT |
| 951 | ACCGCTGGCC AGCATTGTCT CACTTCATGC CTCCAGCTGC GGTGGTAGGC |
| 1001 | TGCAGACCTC ACCCGCACCG ATCCAGACCA CTCCTCCCAA GGACACTTGT |
| 1051 | AGCCCGGAGC TGCTCATGTC CTTGATCCAG ACAAAGTGTG CCGACGACGC |
| 1101 | CATGACCCTG GTACTAAAGA AAGAGCTTGT TGCGCATTTG AAGTGCACCA |
| 1151 | TCACGGGCCT GACCTTCTGG GACCCCAGCT GTGAGGCAGA GGACAGGGGT |
| 1201 | GACAAGTTTG TCTTGCGCAG TGCTTACTCC AGCTGTGGCA TGCAGGTGTC |
| 1251 | AGCAAGTATG ATCAGCAATG AGGCGGTGGT CAATATCCTG TCGAGCTCAT |
| 1301 | CACCACAGCG GAAAAAGGTG CACTGCCTCA ACATGGACAG CCTCTCTTTC |
| 1351 | CAGCTGGGCC TCTACCTCAG CCCACACTTC CTCCAGGCCT CCAACACCAT |
| 1401 | CGAGCCGGGG CAGCAGAGCT TTGTGCAGGT CAGAGTGTCC CCATCCGTCT |
| 1451 | CCGAGTTCCT GCTCCAGTTA GACAGCTGCC ACCTGGACTT GGGGCCTGAG |
| 1501 | GGAGGCACCG TGGAACTCAT CCAGGGCCGG GCGGCCAAGG GCAACTGTGT |
| 1551 | GAGCCTGCTG TCCCCAAGCC CCGAGGGTGA CCCGCGCTTC AGCTTCCTCC |
| 1601 | TCCACTTCTA CACAGTACCC ATACCCAAAA CCGGCACCCT CAGCTGCACG |
| 1651 | GTAGCCCTGC GTCCCAAGAC CGGGTCTCAA GACCAGGAAG TCCATAGGAC |
| 1701 | TGTCTTCATG CGCTTGAACA TCATCAGCCC TGACCTGTCT GGTTGCACAA |
| 1751 | GCAAAGGCCT CGTCCTGCCC GCCGTGCTGG GCATCACCTT TGGTGCCTTC |
| 1801 | CTCATCGGGG CCCTGCTCAC TGCTGCACTC TGGTACATCT ACTCGCACAC |
| 1851 | GCGTTCCCCC AGCAAGCGGG AGCCCGTGGT GGCGGTGGCT GCCCCGGCCT |
| 1901 | CCTCGGAGAG CAGCAGCACC AACCACAGCA TCGGGAGCAC CCAGAGCACC |
| 1951 | CCCTGCTCCA CCAGCAGCAT GGCA |

A nucleic acid sequence encoding a processed extracellular ENG isoform1 polypeptide is as follows (SEQ ID NO: 504):

(SEQ ID NO: 504)

GAAACAGTCCATTGTGACCTTCAGCCTGTGGGCCCCGAGAGGGGCGAGGT
GACATATACCACTAGCCAGGTCTCGAAGGGCTGCGTGGCTCAGGCCCCCA
ATGCCATCCTTGAAGTCCATGTCCTCTTCCTGGAGTTCCCAACGGGCCCG
TCACAGCTGGAGCTGACTCTCCAGGCATCCAAGCAAAATGGCACCTGGCC
CCGAGAGGTGCTTCTGGTCCTCAGTGTAAACAGCAGTGTCTTCCTGCATC
TCCAGGCCCTGGGAATCCCACTGCACTTGGCCTACAATTCCAGCCTGGTC
ACCTTCCAAGAGCCCCCGGGGGTCAACACCACAGAGCTGCCATCCTTCCC
CAAGACCCAGATCCTTGAGTGGGCAGCTGAGAGGGGCCCCATCACCTCTG
CTGCTGAGCTGAATGACCCCCAGAGCATCCTCCTCCGACTGGGCCAAGCC
CAGGGGTCACTGTCCTTCTGCATGCTGGAAGCCAGCCAGGACATGGGCCG
CACGCTCGAGTGGCGGCCGCGTACTCCAGCCTTGGTCCGGGGCTGCCACT
TGGAAGGCGTGGCCGGCCACAAGGAGGCGCACATCCTGAGGGTCCTGCCG
GGCCACTCGGCCGGGCCCCGGACGGTGACGGTGAAGGTGGAACTGAGCTG
CGCACCCGGGGATCTCGATGCCGTCCTCATCCTGCAGGGTCCCCCCTACG
TGTCCTGGCTCATCGACGCCAACCACAACATGCAGATCTGGACCACTGGA
GAATACTCCTTCAAGATCTTTCCAGAGAAAAACATTCGTGGCTTCAAGCT
CCCAGACACACCTCAAGGCCTCCTGGGGGAGGCCCGGATGCTCAATGCCA
GCATTGTGGCATCCTTCGTGGAGCTACCGCTGGCCAGCATTGTCTCACTT
CATGCCTCCAGCTGCGGTGGTAGGCTGCAGACCTCACCCGCACCGATCCA
GACCACTCCTCCCAAGGACACTTGTAGCCCGGAGCTGCTCATGTCCTTGA
TCCAGACAAAGTGTGCCGACGACGCCATGACCCTGGTACTAAAGAAAGAG
CTTGTTGCGCATTTGAAGTGCACCATCACGGGCCTGACCTTCTGGGACCC
CAGCTGTGAGGCAGAGGACAGGGGTGACAAGTTTGTCTTGCGCAGTGCTT
ACTCCAGCTGTGGCATGCAGGTGTCAGCAAGTATGATCAGCAATGAGGCG
GTGGTCAATATCCTGTCGAGCTCATCACCACAGCGGAAAAAGGTGCACTG
CCTCAACATGGACAGCCTCTCTTTCCAGCTGGGCCTCTACCTCAGCCCAC
ACTTCCTCCAGGCCTCCAACACCATCGAGCCGGGGCAGCAGAGCTTTGTG
CAGGTCAGAGTGTCCCCATCCGTCTCCGAGTTCCTGCTCCAGTTAGACAG
CTGCCACCTGGACTTGGGGCCTGAGGGAGGCACCGTGGAACTCATCCAGG
GCCGGGCGGCCAAGGGCAACTGTGTGAGCCTGCTGTCCCCAAGCCCCGAG
GGTGACCCGCGCTTCAGCTTCCTCCTCCACTTCTACACAGTACCCATACC
CAAAACCGGCACCCTCAGCTGCACGGTAGCCCTGCGTCCCAAGACCGGGT
CTCAAGACCAGGAAGTCCATAGGACTGTCTTCATGCGCTTGAACATCATC
AGCCCTGACCTGTCTGGTTGCACAAGCAAAGGC

The human endoglin isoform 2 precursor protein sequence (NCBI Ref Seq NP_000109.1) is as follows:

| | (SEQ ID NO: 505) |
|---|---|
| 1 | MDRGTLPLAV ALLLASCSLS PTSLAETVHC DLQPVGPERG EVTYTTSQVS KGCVAQAPNA |
| 61 | ILEVHVLFLE FPTGPSQLEL TLQASKQNGT WPREVLLVLS VNSSVFLHLQ ALGIPLHLAY |
| 121 | NSSLVTFQEP PGVNTTELPS FPKTQILEWA AERGPITSAA ELNDPQSILL RLGQAQGSLS |
| 181 | FCMLEASQDM GRTLEWRPRT PALVRGCHLE GVAGHKEAHI LRVLPGHSAG PRTVTVKVEL |
| 241 | SCAPGDLDAV LILQGPPYVS WLIDANHNMQ IWTTGEYSFK IFPEKNIRGF KLPDTPQGLL |
| 301 | GEARMLNASI VASFVELPLA SIVSLHASSC GGRLQTSPAP IQTTPPKDTC SPELLMSLIQ |
| 361 | TKCADDAMTL VLKKELVAHL KCTITGLTFW DPSCEAEDRG DKFVLRSAYS SCGMQVSASM |
| 421 | ISNEAVVNIL SSSSPQRKKV HCLNMDSLSF QLGLYLSPHF LQASNTIEPG QQSFVQVRVS |
| 481 | PSVSEFLLQL DSCHLDLGPE GGTVELIQGR AAKGNCVSLL SPSPEGDPRF SFLLHFYTVP |
| 541 | 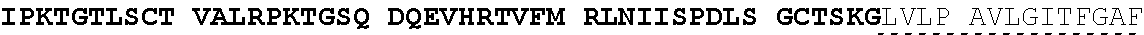 |
| 601 | 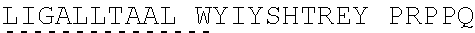 |

The signal peptide is indicated by single underline, the extracellular domain is indicated in bold font, and the transmembrane domain is indicated by dotted underline. The endoglin isoform 2 has a shortened and distinct intracellular domain compared to endoglin isoform 1 and an unchanged extracellular domain compared to endoglin isoform 1.

A processed extracellular endoglin polypeptide sequence (isoform 2) is as follows:

(SEQ ID NO: 506)

ETVHCDLQPVGPERGEVTYTTSQVSKGCVAQAPNAILEVHVLFLEFPTGP
SQLELTLQASKQNGTWPREVLLVLSVNSSVFLHLQALGIPLHLAYNSSLV
TFQEPPGVNTTELPSFPKTQILEWAAERGPITSAAELNDPQSILLRLGQA
QGSLSFCMLEASQDMGRTLEWRPRTPALVRGCHLEGVAGHKEAHILRVLP
GHSAGPRTVTVKVELSCAPGDLDAVLILQGPPYVSWLIDANHNMQIWTTG
EYSFKIFPEKNIRGFKLPDTPQGLLGEARMLNASIVASFVELPLASIVSL
HASSCGGRLQTSPAPIQTTPPKDTCSPELLMSLIQTKCADDAMTLVLKKE
LVAHLKCTITGLTFWDPSCEAEDRGDKFVLRSAYSSCGMQVSASMISNEA
VVNILSSSSPQRKKVHCLNMDSLSFQLGLYLSPHFLQASNTIEPGQQSFV
QVRVSPSVSEFLLQLDSCHLDLGPEGGTVELIQGRAAKGNCVSLLSPSPE
GDPRFSFLLHFYTVPIPKTGTLSCTVALRPKTGSQDQEVHRTVFMRLNII
SPDLSGCTSKG

A nucleic acid sequence encoding unprocessed human ENG isoform 2 precursor protein is shown below (SEQ ID NO: 507), corresponding to nucleotides 419-2293 of NCBI Reference Sequence NM_000118.3. The signal sequence is underlined.

(SEQ ID NO: 507)

ATGGACCGCGGCACGCTCCCTCTGGCTGTTGCCCTGCTGCTGGCCAGCTG
CAGCCTCAGCCCCACAAGTCTTGCAGAAACAGTCCATTGTGACCTTCAGC
CTGTGGGCCCCGAGAGGGGCGAGGTGACATATACCACTAGCCAGGTCTCG
AAGGGCTGCGTGGCTCAGGCCCCCAATGCCATCCTTGAAGTCCATGTCCT
CTTCCTGGAGTTCCCAACGGGCCCGTCACAGCTGGAGCTGACTCTCCAGG
CATCCAAGCAAAATGGCACCTGGCCCCGAGAGGTGCTTCTGGTCCTCAGT
GTAAACAGCAGTGTCTTCCTGCATCTCCAGGCCCTGGGAATCCCACTGCA
CTTGGCCTACAATTCCAGCCTGGTCACCTTCCAAGAGCCCCCGGGGGTCA
ACACCACAGAGCTGCCATCCTTCCCCAAGACCCAGATCCTTGAGTGGGCA
GCTGAGAGGGGCCCCATCACCTCTGCTGCTGAGCTGAATGACCCCCAGAG
CATCCTCCTCCGACTGGGCCAAGCCCAGGGGTCACTGTCCTTCTGCATGC
TGGAAGCCAGCCAGGACATGGGCCGCACGCTCGAGTGGCGGCCGCGTACT
CCAGCCTTGGTCCGGGGCTGCCACTTGGAAGGCGTGGCCGGCCACAAGGA
GGCGCACATCCTGAGGGTCCTGCCGGGCCACTCGGCCGGGCCCCGGACGG
TGACGGTGAAGGTGGAACTGAGCTGCGCACCCGGGGATCTCGATGCCGTC
CTCATCCTGCAGGGTCCCCCCTACGTGTCCTGGCTCATCGACGCCAACCA
CAACATGCAGATCTGGACCACTGGAGAATACTCCTTCAAGATCTTTCCAG
AGAAAAACATTCGTGGCTTCAAGCTCCCAGACACACCTCAAGGCCTCCTG
GGGGAGGCCCGGATGCTCAATGCCAGCATTGTGGCATCCTTCGTGGAGCT
ACCGCTGGCCAGCATTGTCTCACTTCATGCCTCCAGCTGCGGTGGTAGGC
TGCAGACCTCACCCGCACCGATCCAGACCACTCCTCCCAAGGACACTTGT
AGCCCGGAGCTGCTCATGTCCTTGATCCAGACAAAGTGTGCCGACGACGC
CATGACCCTGGTACTAAAGAAAGAGCTTGTTGCGCATTTGAAGTGCACCA
TCACGGGCCTGACCTTCTGGGACCCCAGCTGTGAGGCAGAGGACAGGGGT
GACAAGTTTGTCTTGCGCAGTGCTTACTCCAGCTGTGGCATGCAGGTGTC
AGCAAGTATGATCAGCAATGAGGCGGTGGTCAATATCCTGTCGAGCTCAT
CACCACAGCGGAAAAAGGTGCACTGCCTCAACATGGACAGCCTCTCTTTC
CAGCTGGGCCTCTACCTCAGCCCACACTTCCTCCAGGCCTCCAACACCAT
CGAGCCGGGGCAGCAGAGCTTTGTGCAGGTCAGAGTGTCCCCATCCGTCT
CCGAGTTCCTGCTCCAGTTAGACAGCTGCCACCTGGACTTGGGGCCTGAG
GGAGGCACCGTGGAACTCATCCAGGGCCGGGCGGCCAAGGGCAACTGTGT
GAGCCTGCTGTCCCCAAGCCCCGAGGGTGACCCGCGCTTCAGCTTCCTCC
TCCACTTCTACACAGTACCCATACCCAAAACCGGCACCCTCAGCTGCACG
GTAGCCCTGCGTCCCAAGACCGGGTCTCAAGACCAGGAAGTCCATAGGAC
TGTCTTCATGCGCTTGAACATCATCAGCCCTGACCTGTCTGGTTGCACAA
GCAAAGGCCTCGTCCTGCCCGCCGTGCTGGGCATCACCTTTGGTGCCTTC
CTCATCGGGGCCCTGCTCACTGCTGCACTCTGGTACATCTACTCGCACAC
GCGTGAGTACCCCAGGCCCCCACAG

A nucleic acid sequence encoding a processed extracellular ENG isoform 2 polypeptide is as follows (SEQ ID NO: 508):

(SEQ ID NO: 508)

GAAACAGTCCATTGTGACCTTCAGCCTGTGGGCCCCGAGAGGGGCGAGGT
GACATATACCACTAGCCAGGTCTCGAAGGGCTGCGTGGCTCAGGCCCCCA
ATGCCATCCTTGAAGTCCATGTCCTCTTCCTGGAGTTCCCAACGGGCCCG
TCACAGCTGGAGCTGACTCTCCAGGCATCCAAGCAAAATGGCACCTGGCC
CCGAGAGGTGCTTCTGGTCCTCAGTGTAAACAGCAGTGTCTTCCTGCATC
TCCAGGCCCTGGGAATCCCACTGCACTTGGCCTACAATTCCAGCCTGGTC
ACCTTCCAAGAGCCCCCGGGGGTCAACACCACAGAGCTGCCATCCTTCCC
CAAGACCCAGATCCTTGAGTGGGCAGCTGAGAGGGGCCCCATCACCTCTG
CTGCTGAGCTGAATGACCCCCAGAGCATCCTCCTCCGACTGGGCCAAGCC
CAGGGGTCACTGTCCTTCTGCATGCTGGAAGCCAGCCAGGACATGGGCCG
CACGCTCGAGTGGCGGCCGCGTACTCCAGCCTTGGTCCGGGGCTGCCACT
TGGAAGGCGTGGCCGGCCACAAGGAGGCGCACATCCTGAGGGTCCTGCCG
GGCCACTCGGCCGGGCCCCGGACGGTGACGGTGAAGGTGGAACTGAGCTG
CGCACCCGGGGATCTCGATGCCGTCCTCATCCTGCAGGGTCCCCCCTACG
TGTCCTGGCTCATCGACGCCAACCACAACATGCAGATCTGGACCACTGGA
GAATACTCCTTCAAGATCTTTCCAGAGAAAAACATTCGTGGCTTCAAGCT
CCCAGACACACCTCAAGGCCTCCTGGGGGAGGCCCGGATGCTCAATGCCA
GCATTGTGGCATCCTTCGTGGAGCTACCGCTGGCCAGCATTGTCTCACTT
CATGCCTCCAGCTGCGGTGGTAGGCTGCAGACCTCACCCGCACCGATCCA
GACCACTCCTCCCAAGGACACTTGTAGCCCGGAGCTGCTCATGTCCTTGA
TCCAGACAAAGTGTGCCGACGACGCCATGACCCTGGTACTAAAGAAAGAG
CTTGTTGCGCATTTGAAGTGCACCATCACGGGCCTGACCTTCTGGGACCC
CAGCTGTGAGGCAGAGGACAGGGGTGACAAGTTTGTCTTGCGCAGTGCTT
ACTCCAGCTGTGGCATGCAGGTGTCAGCAAGTATGATCAGCAATGAGGCG
GTGGTCAATATCCTGTCGAGCTCATCACCACAGCGGAAAAAGGTGCACTG
CCTCAACATGGACAGCCTCTCTTTCCAGCTGGGCCTCTACCTCAGCCCAC
ACTTCCTCCAGGCCTCCAACACCATCGAGCCGGGGCAGCAGAGCTTTGTG
CAGGTCAGAGTGTCCCCATCCGTCTCCGAGTTCCTGCTCCAGTTAGACAG
CTGCCACCTGGACTTGGGGCCTGAGGGAGGCACCGTGGAACTCATCCAGG
GCCGGGCGGCCAAGGGCAACTGTGTGAGCCTGCTGTCCCCAAGCCCCGAG
GGTGACCCGCGCTTCAGCTTCCTCCTCCACTTCTACACAGTACCCATACC
CAAAACCGGCACCCTCAGCTGCACGGTAGCCCTGCGTCCCAAGACCGGGT
CTCAAGACCAGGAAGTCCATAGGACTGTCTTCATGCGCTTGAACATCATC
AGCCCTGACCTGTCTGGTTGCACAAGCAAAGGC

An alternative processed extracellular endoglin polypeptide sequence (from either isoform 1 or isoform 2) is as follows:

(SEQ ID NO: 593)

ETVHCDLQPVGPERGEVTYTTSQVSKGCVAQAPNAILEVHVLFLEFPTGP
SQLELTLQASKQNGTWPREVLLVLSVNSSVFLHLQALGIPLHLAYNSSLV
TFQEPPGVNTTELPSFPKTQILEWAAERGPITSAAELNDPQSILLRLGQA
QGSLSFCMLEASQDMGRTLEWRPRTPALVRGCHLEGVAGHKEAHILRVLP
GHSAGPRTVTVKVELSCAPGDLDAVLILQGPPYVSWLIDANHNMQIWTTG
EYSFKIFPEKNIRGFKLPDTPQGLLGEARMLNASIVASFVELPLASIVSL
HASSCGGRLQTSPAPIQTTPP

A nucleic acid sequence encoding this alternative processed extracellular ENG polypeptide is as follows (SEQ ID NO: 594):

(SEQ ID NO: 594)

GAAACAGTCCATTGTGACCTTCAGCCTGTGGGCCCCGAGAGGGGCGAGGT
GACATATACCACTAGCCAGGTCTCGAAGGGCTGCGTGGCTCAGGCCCCCA
ATGCCATCCTTGAAGTCCATGTCCTCTTCCTGGAGTTCCCAACGGGCCCG
TCACAGCTGGAGCTGACTCTCCAGGCATCCAAGCAAAATGGCACCTGGCC
CCGAGAGGTGCTTCTGGTCCTCAGTGTAAACAGCAGTGTCTTCCTGCATC
TCCAGGCCCTGGGAATCCCACTGCACTTGGCCTACAATTCCAGCCTGGTC
ACCTTCCAAGAGCCCCCGGGGGTCAACACCACAGAGCTGCCATCCTTCCC
CAAGACCCAGATCCTTGAGTGGGCAGCTGAGAGGGGCCCCATCACCTCTG
CTGCTGAGCTGAATGACCCCCAGAGCATCCTCCTCCGACTGGGCCAAGCC
CAGGGGTCACTGTCCTTCTGCATGCTGGAAGCCAGCCAGGACATGGGCCG
CACGCTCGAGTGGCGGCCGCGTACTCCAGCCTTGGTCCGGGGCTGCCACT
TGGAAGGCGTGGCCGGCCACAAGGAGGCGCACATCCTGAGGGTCCTGCCG
GGCCACTCGGCCGGGCCCCGGACGGTGACGGTGAAGGTGGAACTGAGCTG
CGCACCCGGGGATCTCGATGCCGTCCTCATCCTGCAGGGTCCCCCCTACG
TGTCCTGGCTCATCGACGCCAACCACAACATGCAGATCTGGACCACTGGA
GAATACTCCTTCAAGATCTTTCCAGAGAAAAACATTCGTGGCTTCAAGCT
CCCAGACACACCTCAAGGCCTCCTGGGGGAGGCCCGGATGCTCAATGCCA
GCATTGTGGCATCCTTCGTGGAGCTACCGCTGGCCAGCATTGTCTCACTT
CATGCCTCCAGCTGCGGTGGTAGGCTGCAGACCTCACCCGCACCGATCCA
GACCACTCCTCCC

The human endoglin isoform 3 protein sequence (NCBI Ref Seq NP_001265067.1) is as follows:

| | (SEQ ID NO: 509) |
|---|---|
| 1 | MLEASQDMGR TLEWRPRTPA LVRGCHLEGV AGHKEAHILR VLPGHSAGPR TVTVKVELSC |
| 61 | APGDLDAVLI LQGPPYVSWL IDANHNMQIW TTGEYSFKIF PEKNIRGFKL PDTPQGLLGE |
| 121 | ARMLNASIVA SFVELPLASI VSLHASSCGG RLQTSPAPIQ TTPPKDTCSP ELLMSLIQTK |
| 181 | CADDAMTLVL KKELVAHLKC TITGLTFWDP SCEAEDRGDK FVLRSAYSSC GMQVSASMIS |
| 241 | NEAVVNILSS SSPQRKKVHC LMMDSLSFQL GLYLSPHFLQ ASNTIEPGQQ SFVQVRVSPS |
| 301 | VSEFLLQLDS CHLDLGPEGG TVELIQGRAA KGNCVSLLSP SPEGDPRFSF LLHFYTVPIP |
| 361 | 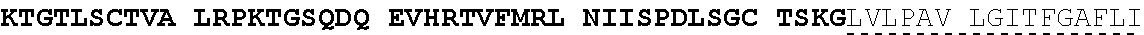 |
| 421 | 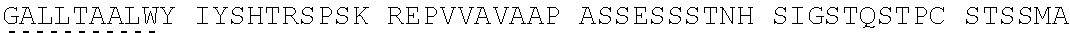 |

The extracellular domain is indicated in bold font, and the transmembrane domain is indicated by dotted underline. The endoglin isoform 3 has a distinct 5′ untranslated region, lacks a portion of the 5′ coding region, and uses a downstream start codon compared to endoglin isoform 1.

A processed extracellular endoglin polypeptide sequence (isoform 3) is as follows:

(SEQ ID NO: 510)

MLEASQDMGRTLEWRPRTPALVRGCHLEGVAGHKEAHILRVLPGHSAGPR
TVTVKVELSCAPGDLDAVLILQGPPYVSWLIDANHNMQIWTTGEYSFKIF
PEKNIRGFKLPDTPQGLLGEARMLNASIVASFVELPLASIVSLHASSCGG
RLQTSPAPIQTTPPKDTCSPELLMSLIQTKCADDAMTLVLKKELVAHLKC
TITGLTFWDPSCEAEDRGDKFVLRSAYSSCGMQVSASMISNEAVVNILSS
SSPQRKKVHCLNMDSLSFQLGLYLSPHFLQASNTIEPGQQSFVQVRVSPS
VSEFLLQLDSCHLDLGPEGGTVELIQGRAAKGNCVSLLSPSPEGDPRFSF
LLHFYTVPIPKTGTLSCTVALRPKTGSQDQEVHRTVFMRLNIISPDLSGC
TSKG

A nucleic acid sequence encoding human ENG isoform 3 protein is shown below (SEQ ID NO: 511), corresponding to nucleotides 705-2132 of NCBI Reference Sequence NM_001278138.1. The transmembrane region is indicated by dotted underline.

(SEQ ID NO: 511)
ATGCTGGAAGCCAGCCAGGACATGGGCCGCACGCTCGAGTGGCGGCCGCGTACTCCAGCCTTGGTCCGGGGCTGC
CACTTGGAAGGCGTGGCCGGCCACAAGGAGGCGCACATCCTGAGGGTCCTGCCGGGCCACTCGGCCGGGCCCCGG
ACGGTGACGGTGAAGGTGGAACTGAGCTGCGCACCCGGGGATCTCGATGCCGTCCTCATCCTGCAGGGTCCCCCC
TACGTGTCCTGGCTCATCGACGCCAACCACAACATGCAGATCTGGACCACTGGAGAATACTCCTTCAAGATCTTT
CCAGAGAAAAACATTCGTGGCTTCAAGCTCCCAGACACACCTCAAGGCCTCCTGGGGGAGGCCCGGATGCTCAAT
GCCAGCATTGTGGCATCCTTCGTGGAGCTACCGCTGGCCAGCATTGTCTCACTTCATGCCTCCAGCTGCGGTGGT
AGGCTGCAGACCTCACCCGCACCGATCCAGACCACTCCTCCCAAGGACACTTGTAGCCCGGAGCTGCTCATGTCC
TTGATCCAGACAAAGTGTGCCGACGACGCCATGACCCTGGTACTAAAGAAAGAGCTTGTTGCGCATTTGAAGTGC
ACCATCACGGGCCTGACCTTCTGGGACCCCAGCTGTGAGGCAGAGGACAGGGGTGACAAGTTTGTCTTGCGCAGT
GCTTACTCCAGCTGTGGCATGCAGGTGTCAGCAAGTATGATCAGCAATGAGGCGGTGGTCAATATCCTGTCGAGC
TCATCACCACAGCGGAAAAAGGTGCACTGCCTCAACATGGACAGCCTCTCTTTCCAGCTGGGCCTCTACCTCAGC
CCACACTTCCTCCAGGCCTCCAACACCATCGAGCCGGGGCAGCAGAGCTTTGTGCAGGTCAGAGTGTCCCCATCC
GTCTCCGAGTTCCTGCTCCAGTTAGACAGCTGCCACCTGGACTTGGGGCCTGAGGGAGGCACCGTGGAACTCATC
CAGGGCCGGGCGGCCAAGGGCAACTGTGTGAGCCTGCTGTCCCCAAGCCCCGAGGGTGACCCGCGCTTCAGCTTC
CTCCTCCACTTCTACACAGTACCCATACCCAAAACCGGCACCCTCAGCTGCACGGTAGCCCTGCGTCCCAAGACC
GGGTCTCAAGACCAGGAAGTCCATAGGACTGTCTTCATGCGCTTGAACATCATCAGCCCTGACCTGTCTGGTTGC

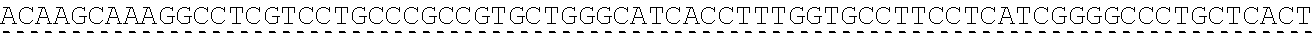

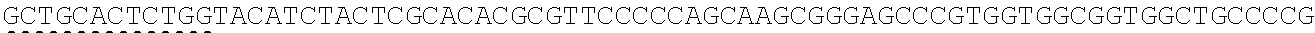

GCCTCCTCGGAGAGCAGCAGCACCAACCACAGCATCGGGAGCACCCAGAGCACCCCCTGCTCCACCAGCAGCATG
GCA

A nucleic acid sequence encoding a processed extracellular ENG isoform 3 polypeptide is as follows (SEQ ID NO: 512):

(SEQ ID NO: 512)

ATGCTGGAAGCCAGCCAGGACATGGGCCGCACGCTCGAGTGGCGGCCGCG
TACTCCAGCCTTGGTCCGGGGCTGCCACTTGGAAGGCGTGGCCGGCCACA
AGGAGGCGCACATCCTGAGGGTCCTGCCGGGCCACTCGGCCGGGCCCCGG
ACGGTGACGGTGAAGGTGGAACTGAGCTGCGCACCCGGGGATCTCGATGC
CGTCCTCATCCTGCAGGGTCCCCCCTACGTGTCCTGGCTCATCGACGCCA
ACCACAACATGCAGATCTGGACCACTGGAGAATACTCCTTCAAGATCTTT
CCAGAGAAAAACATTCGTGGCTTCAAGCTCCCAGACACACCTCAAGGCCT
CCTGGGGGAGGCCCGGATGCTCAATGCCAGCATTGTGGCATCCTTCGTGG
AGCTACCGCTGGCCAGCATTGTCTCACTTCATGCCTCCAGCTGCGGTGGT
AGGCTGCAGACCTCACCCGCACCGATCCAGACCACTCCTCCCAAGGACAC
TTGTAGCCCGGAGCTGCTCATGTCCTTGATCCAGACAAAGTGTGCCGACG
ACGCCATGACCCTGGTACTAAAGAAAGAGCTTGTTGCGCATTTGAAGTGC
ACCATCACGGGCCTGACCTTCTGGGACCCCAGCTGTGAGGCAGAGGACAG
GGGTGACAAGTTTGTCTTGCGCAGTGCTTACTCCAGCTGTGGCATGCAGG
TGTCAGCAAGTATGATCAGCAATGAGGCGGTGGTCAATATCCTGTCGAGC
TCATCACCACAGCGGAAAAAGGTGCACTGCCTCAACATGGACAGCCTCTC
TTTCCAGCTGGGCCTCTACCTCAGCCCACACTTCCTCCAGGCCTCCAACA
CCATCGAGCCGGGGCAGCAGAGCTTTGTGCAGGTCAGAGTGTCCCCATCC
GTCTCCGAGTTCCTGCTCCAGTTAGACAGCTGCCACCTGGACTTGGGGCC
TGAGGGAGGCACCGTGGAACTCATCCAGGGCCGGGCGGCCAAGGGCAACT
GTGTGAGCCTGCTGTCCCCAAGCCCCGAGGGTGACCCGCGCTTCAGCTTC
CTCCTCCACTTCTACACAGTACCCATACCCAAAACCGGCACCCTCAGCTG
CACGGTAGCCCTGCGTCCCAAGACCGGGTCTCAAGACCAGGAAGTCCATA
GGACTGTCTTCATGCGCTTGAACATCATCAGCCCTGACCTGTCTGGTTGC

In certain embodiments, the disclosure relates to heteromultimers that comprise at least one endoglin polypeptide, which includes fragments, functional variants, and modified forms thereof. Preferably, endoglin polypeptides for use in accordance with the disclosure (e.g., heteromultimers comprising an endoglin polypeptide and uses thereof) are soluble (e.g., an extracellular domain of endoglin). In other preferred embodiments, endoglin polypeptides for use in accordance with the disclosure bind to and/or inhibit (antagonize) activity (e.g., Smad signaling) of one or more TGF-beta superfamily ligands. In some embodiments, heteromultimers of the disclosure comprise at least one endoglin polypeptide that is at least 70%, 75%, 80%, 85%, 90%, 91%, 92%, 93%, 94%, 95%, 96%, 97%, 98%, 99%, or 100% identical to the amino acid sequence of SEQ ID NOs: 501, 502, 505, 506, 509, 510, or 593. In some embodiments, heteromultimers of the disclosure comprise at least one endoglin polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 26-30 (e.g., amino acid residues 26, 27, 28, 29, or 30) of SEQ ID NO: 501, and ends at any one of amino acids 330-346 (e.g., amino acid residues 330, 331, 332, 333, 334, 335, 336, 337, 338, 339, 340, 341, 342, 343, 344, 345, or 346) of SEQ ID NO: 501. In some embodiments, heteromultimers of the disclosure comprise at least one endoglin polypeptide that is at least 70%, 75%, 80%, 85%, 90%, 91%, 92%, 93%, 94%, 95%, 96%, 97%, 98%, 99%, or 100% identical to amino acids of 26-346 of SEQ ID NO: 501. In some embodiments, heteromultimers of the disclosure comprise at least one endoglin polypeptide that is at least 70%, 75%, 80%, 85%, 90%, 91%, 92%, 93%, 94%, 95%, 96%, 97%, 98%, 99%, or 100% identical to amino acids of 30-330 of SEQ ID NO: 501. In some embodiments, heteromultimers of the disclosure comprise at least one endoglin polypeptide that is at least 70%, 75%, 80%, 85%, 90%, 91%, 92%, 93%, 94%, 95%, 96%, 97%, 98%, 99%, or 100% identical to amino acids of 26-330 of SEQ ID NO: 501. In some embodiments, heteromultimers of the disclosure comprise at least one endoglin polypeptide that is at least 70%, 75%, 80%, 85%, 90%, 91%, 92%, 93%, 94%, 95%, 96%, 97%, 98%, 99%, or 100% identical to amino acids of 30-346 of SEQ ID NO: 501. In some embodiments, heteromultimers of the disclosure comprise at least one endoglin polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 26-30 (e.g., amino acid residues 26, 27, 28, 29, or 30) of SEQ ID NO: 505, and ends at any one of amino acids 330-346 (e.g., amino acid residues 330, 331, 332, 333, 334, 335, 336, 337, 338, 339, 340, 341, 342, 343, 344, 345, or 346) of SEQ ID NO: 505. In some embodiments, heteromultimers of the disclosure comprise at least one endoglin polypeptide that is at least 70%, 75%, 80%, 85%, 90%, 91%, 92%, 93%, 94%, 95%, 96%, 97%, 98%, 99%, or 100% identical to amino acids of 26-346 of SEQ ID NO: 505. In some embodiments, heteromultimers of the disclosure comprise least one endoglin polypeptide that is at least 70%, 75%, 80%, 85%, 90%, 91%, 92%, 93%, 94%, 95%, 96%, 97%, 98%, 99%, or 100% identical to amino acids of 30-330 of SEQ ID NO: 505. In some embodiments, heteromultimers of the disclosure comprise at least one endoglin polypeptide that is at least 70%, 75%, 80%, 85%, 90%, 91%, 92%, 93%, 94%, 95%, 96%, 97%, 98%, 99%, or 100% identical to amino acids of 26-330 of SEQ ID NO: 505. In some embodiments, heteromultimers of the disclosure comprise least one endoglin polypeptide that is at least 70%, 75%, 80%, 85%, 90%, 91%, 92%, 93%, 94%, 95%, 96%, 97%, 98%, 99%, or 100% identical to amino acids of 30-346 of SEQ ID NO: 505. In some embodiments, heteromultimers of the disclosure comprise at least one endoglin polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 1-25 (e.g., amino acid residues 1, 2, 3, 4, 5, 6, 7, 8, 9, 10, 11, 12, 13, 14, 15, 16, 17, 18, 19, 20, 21, 22, 23, 24, or 25) of SEQ ID NO: 509, and ends at any one of amino acids 148-164 (e.g., amino acid residues 148, 149, 150, 151, 152, 153, 154, 155, 156, 157, 158, 159, 160, 161, 162, 163, or 164) of SEQ ID NO: 509. In some embodiments, heteromultimers of the disclosure comprise at least one endoglin polypeptide that is at least 70%, 75%, 80%, 85%, 90%, 91%, 92%, 93%, 94%, 95%, 96%, 97%, 98%, 99%, or 100% identical to amino acids of 1-164 of SEQ ID NO: 509. In some embodiments, heteromultimers of the disclosure comprise at least one endoglin polypeptide that is at least 70%, 75%, 80%, 85%, 90%, 91%, 92%, 93%, 94%, 95%, 96%, 97%, 98%, 99%, or 100% identical to amino acids of 25-148 of SEQ ID NO: 509. In some embodiments, heteromultimers of the disclosure comprise at least one endoglin polypeptide that is at least 70%, 75%, 80%, 85%, 90%, 91%, 92%, 93%, 94%, 95%, 96%, 97%, 98%, 99%, or 100% identical to amino acids of 1-148 of SEQ ID NO: 509. In some embodiments, heteromultimers of the disclosure comprise at least one endoglin polypeptide that is at least 70%, 75%, 80%, 85%, 90%, 91%, 92%, 93%, 94%, 95%, 96%, 97%, 98%, 99%, or 100% identical to amino acids of 25-164 of SEQ ID NO: 509. In some embodiments, heteromultimers of the disclosure comprise at least one endoglin polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 26-30 (e.g., amino acid residues 26, 27, 28, 29, or 30) of SEQ ID NO: 501, and ends at any one of amino acids 582-586 (e.g., amino acid residues 582, 583, 584, 585, or 586) of SEQ ID NO: 501. In some embodiments, heteromultimers of the disclosure comprise at least one endoglin polypeptide that is at least 70%, 75%, 80%, 85%, 90%, 91%, 92%, 93%, 94%, 95%, 96%, 97%, 98%, 99%, or 100% identical to amino acids of 26-586 of SEQ ID NO: 501. In some embodiments, heteromultimers of the disclosure comprise at least one endoglin polypeptide that is at least 70%, 75%, 80%, 85%, 90%, 91%, 92%, 93%, 94%, 95%, 96%, 97%, 98%, 99%, or 100% identical to amino acids of 30-582 of SEQ ID NO: 501. In some embodiments, heteromultimers of the disclosure comprise at least one endoglin polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 26-30 (e.g., amino acid residues 26, 27, 28, 29, or 30) of SEQ ID NO: 505, and ends at any one of amino acids 582-586 (e.g., amino acid residues 582, 583, 584, 585, or 586) of SEQ ID NO: 505. In some embodiments, heteromultimers of the disclosure comprise at least one endoglin polypeptide that is at least 70%, 75%, 80%, 85%, 90%, 91%, 92%, 93%, 94%, 95%, 96%, 97%, 98%, 99%, or 100% identical to amino acids of 26-586 of SEQ ID NO: 505. In some embodiments, heteromultimers of the disclosure comprise at least one endoglin polypeptide that is at least 70%, 75%, 80%, 85%, 90%, 91%, 92%, 93%, 94%, 95%, 96%, 97%, 98%, 99%, or 100% identical to amino acids of 30-582 of SEQ ID NO: 505. In some embodiments, heteromultimers of the disclosure comprise at least one endoglin polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 1-25 (e.g., amino acid residues 1, 2, 3, 4, 5, 6, 7, 8, 9, 10, 11, 12, 13, 14, 15, 16, 17, 18, 19, 20, 21, 22, 23, 24, or 25) of SEQ ID NO: 509, and ends at any one of amino acids 400-404 (e.g., amino acid residues 400, 401, 402, or 403) of SEQ ID NO: 509. In some embodiments, heteromultimers of the disclosure comprise at least one endoglin polypeptide that is at least 70%, 75%, 80%, 85%, 90%, 91%, 92%, 93%, 94%, 95%, 96%, 97%, 98%, 99%, or 100% identical to amino acids of 1-404 of SEQ ID NO: 509. In some embodiments, heteromultimers of the disclosure comprise at least one endoglin polypeptide that is at least 70%, 75%, 80%, 85%, 90%, 91%, 92%, 93%, 94%, 95%, 96%, 97%, 98%, 99%, or 100% identical to amino acids of 25-400 of SEQ ID NO: 509.

The term “Cripto-1 polypeptide” includes polypeptides comprising any naturally occurring Cripto-1 protein (encoded by TDGF1 or one of its nonhuman orthologs) as well as any variants thereof (including mutants, fragments, fusions, and peptidomimetic forms) that retain a useful activity.

The human Cripto-1 isoform 1 precursor protein sequence (NCBI Ref Seq NP_003203.1) is as follows:

| (SEQ ID NO: 513) | |
|---|---|
| 1 | MDCRKMARFS YSVIWIMAIS KVFELGLVAG LGHQEFARPS RGYLAFRDDS IWPQEEPAIR |
| 61 | PRSSQRVPPM GIQHSKELNR TCCLNGGTCM LGSFCACPPS FYGRNCEHDV RKENCGSVPH |
| 121 | DTWLPKKCSL CKCWHGQLRC FPQAFLPGCD GLVMDEHLVA SRTPELPPSA RTTTFMLVGI |
| 181 | CLSIQSYY |

The signal peptide is indicated by single underline.

A processed Cripto-1 isoform 1 polypeptide sequence is as follows:

(SEQ ID NO: 514)

LGHQEFARPSRGYLAFRDDSIWPQEEPAIRPRSSQRVPPMGIQHSKELNR
TCCLNGGTCMLGSFCACPPSFYGRNCEHDVRKENCGSVPHDTWLPKKCSL
CKCWHGQLRCFPQAFLPGCDGLVMDEHLVAS

A nucleic acid sequence encoding unprocessed human Cripto-1 isoform 1 precursor protein is shown below (SEQ ID NO: 515), corresponding to nucleotides 385-948 of NCBI Reference Sequence NM_003212.3. The signal sequence is underlined.

(SEQ ID NO: 515)

ATGGACTGCAGGAAGATGGCCCGCTTCTCTTACAGTGTGATTTGGATCAT
GGCCATTTCTAAAGTCTTTGAACTGGGATTAGTTGCCGGGCTGGGCCATC
AGGAATTTGCTCGTCCATCTCGGGGATACCTGGCCTTCAGAGATGACAGC
ATTTGGCCCCAGGAGGAGCCTGCAATTCGGCCTCGGTCTTCCCAGCGTGT
GCCGCCCATGGGGATACAGCACAGTAAGGAGCTAAACAGAACCTGCTGCC
TGAATGGGGGAACCTGCATGCTGGGGTCCTTTTGTGCCTGCCCTCCCTCC
TTCTACGGACGGAACTGTGAGCACGATGTGCGCAAAGAGAACTGTGGGTC
TGTGCCCCATGACACCTGGCTGCCCAAGAAGTGTTCCCTGTGTAAATGCT
GGCACGGTCAGCTCCGCTGCTTTCCTCAGGCATTTCTACCCGGCTGTGAT
GGCCTTGTGATGGATGAGCACCTCGTGGCTTCCAGGACTCCAGAACTACC
ACCGTCTGCACGTACTACCACTTTTATGCTAGTTGGCATCTGCCTTTCTA
TACAAAGCTACTAT

A nucleic acid sequence encoding a processed Cripto-1 isoform 1 is shown below (SEQ ID NO: 516):

(SEQ ID NO: 516)

CTGGGCCATCAGGAATTTGCTCGTCCATCTCGGGGATACCTGGCCTTCAG
AGATGACAGCATTTGGCCCCAGGAGGAGCCTGCAATTCGGCCTCGGTCTT
CCCAGCGTGTGCCGCCCATGGGGATACAGCACAGTAAGGAGCTAAACAGA
ACCTGCTGCCTGAATGGGGGAACCTGCATGCTGGGGTCCTTTTGTGCCTG
CCCTCCCTCCTTCTACGGACGGAACTGTGAGCACGATGTGCGCAAAGAGA
ACTGTGGGTCTGTGCCCCATGACACCTGGCTGCCCAAGAAGTGTTCCCTG
TGTAAATGCTGGCACGGTCAGCTCCGCTGCTTTCCTCAGGCATTTCTACC
CGGCTGTGATGGCCTTGTGATGGATGAGCACCTCGTGGCTTCC

The human Cripto-1 isoform 2 protein sequence (NCBI Ref Seq NP_001167607.1) is as follows:

| (SEQ ID NO: 517) | |
|---|---|
| 1 | MAISKVFELG LVAGLGHQEF ARPSRGYLAF RDDSIWPQEE PAIRPRSSQR VPPMGIQHSK |
| 61 | ELNRTCCLNG GTCMLGSFCA CPPSFYGRNC EHDVRKENCG SVPHDTWLPK KCSLCKCWHG |
| 121 | QLRCFPQAFL PGCDGLVMDE HLVASRTPEL PPSARTTTFM LVGICLSIQS YY |

A mature Cripto-1 polypeptide sequence (isoform 2) is as follows:

(SEQ ID NO: 518)

MAISKVFELGLVAGLGHQEFARPSRGYLAFRDDSIWPQEEPAIRPRSSQR
VPPMGIQHSKELNRTCCLNGGTCMLGSFCACPPSFYGRNCEHDVRKENCG
SVPHDTWLPKKCSLCKCWHGQLRCFPQAFLPGCDGLVMDEHLVAS

A nucleic acid sequence encoding unprocessed human Cripto-1 isoform 2 precursor protein is shown below (SEQ ID NO: 519), corresponding to nucleotides 43-558 of NCBI Reference Sequence NM_001174136.1.

(SEQ ID NO: 519)

ATGGCCATTTCTAAAGTCTTTGAACTGGGATTAGTTGCCGGGCTGGGCCA
TCAGGAATTTGCTCGTCCATCTCGGGGATACCTGGCCTTCAGAGATGACA
GCATTTGGCCCCAGGAGGAGCCTGCAATTCGGCCTCGGTCTTCCCAGCGT
GTGCCGCCCATGGGGATACAGCACAGTAAGGAGCTAAACAGAACCTGCTG
CCTGAATGGGGGAACCTGCATGCTGGGGTCCTTTTGTGCCTGCCCTCCCT
CCTTCTACGGACGGAACTGTGAGCACGATGTGCGCAAAGAGAACTGTGGG
TCTGTGCCCCATGACACCTGGCTGCCCAAGAAGTGTTCCCTGTGTAAATG
CTGGCACGGTCAGCTCCGCTGCTTTCCTCAGGCATTTCTACCCGGCTGTG
ATGGCCTTGTGATGGATGAGCACCTCGTGGCTTCCAGGACTCCAGAACTA
CCACCGTCTGCACGTACTACCACTTTTATGCTAGTTGGCATCTGCCTTTC
TATACAAAGCTACTAT

A nucleic acid sequence encoding a processed human Cripto-1 isoform 2 is shown below (SEQ ID NO: 520):

(SEQ ID NO: 520)

ATGGCCATTTCTAAAGTCTTTGAACTGGGATTAGTTGCCGGGCTGGGCCA
TCAGGAATTTGCTCGTCCATCTCGGGGATACCTGGCCTTCAGAGATGACA
GCATTTGGCCCCAGGAGGAGCCTGCAATTCGGCCTCGGTCTTCCCAGCGT
GTGCCGCCCATGGGGATACAGCACAGTAAGGAGCTAAACAGAACCTGCTG
CCTGAATGGGGGAACCTGCATGCTGGGGTCCTTTTGTGCCTGCCCTCCCT
CCTTCTACGGACGGAACTGTGAGCACGATGTGCGCAAAGAGAACTGTGGG
TCTGTGCCCCATGACACCTGGCTGCCCAAGAAGTGTTCCCTGTGTAAATG
CTGGCACGGTCAGCTCCGCTGCTTTCCTCAGGCATTTCTACCCGGCTGTG
ATGGCCTTGTGATGGATGAGCACCTCGTGGCTTCC

In certain embodiments, the disclosure relates to heteromultimers that comprise at least one Cripto-1 polypeptide, which includes fragments, functional variants, and modified forms thereof. Preferably, Cripto-1 polypeptides for use in accordance with the disclosure (e.g., heteromultimers comprising a Cripto-1 polypeptide and uses thereof) are soluble (e.g., an extracellular domain of Cripto-1). In other preferred embodiments, Cripto-1 polypeptides for use in accordance with the disclosure bind to and/or inhibit (antagonize) activity (e.g., Smad signaling) of one or more TGF-beta superfamily ligands. In some embodiments, heteromultimers of the disclosure comprise at least one Cripto-1 polypeptide that is at least 70%, 75%, 80%, 85%, 90%, 91%, 92%, 93%, 94%, 95%, 96%, 97%, 98%, 99%, or 100% identical to the amino acid sequence of SEQ ID NOs: 513, 514, 517, or 518. In some embodiments, heteromultimers of the disclosure comprise at least one Cripto-1 polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 31-82 (e.g., amino acid residues 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, or 82) of SEQ ID NO: 513, and ends at any one of amino acids 172-188 (e.g., amino acid residues 172, 173, 174, 175, 176, 177, 178, 179, 180, 181, 182, 183, 184, 185, 186, 187, or 188) of SEQ ID NO: 513. In some embodiments, heteromultimers of the disclosure comprise at least one Cripto-1 polypeptide that is at least 70%, 75%, 80%, 85%, 90%, 91%, 92%, 93%, 94%, 95%, 96%, 97%, 98%, 99%, or 100% identical to amino acids of 31-188 of SEQ ID NO: 513. In some embodiments, heteromultimers of the disclosure comprise at least one Cripto-1 polypeptide that is at least 70%, 75%, 80%, 85%, 90%, 91%, 92%, 93%, 94%, 95%, 96%, 97%, 98%, 99%, or 100% identical to amino acids of 63-172 of SEQ ID NO: 513. In some embodiments, heteromultimers of the disclosure comprise at least one Cripto-1 polypeptide that is at least 70%, 75%, 80%, 85%, 90%, 91%, 92%, 93%, 94%, 95%, 96%, 97%, 98%, 99%, or 100% identical to amino acids of 82-172 of SEQ ID NO: 513. In some embodiments, heteromultimers of the disclosure comprise at least one Cripto-1 polypeptide that is at least 70%, 75%, 80%, 85%, 90%, 91%, 92%, 93%, 94%, 95%, 96%, 97%, 98%, 99%, or 100% identical to amino acids of 82-188 of SEQ ID NO: 513. In some embodiments, heteromultimers of the disclosure comprise at least one Cripto-1 polypeptide that is at least 70%, 75%, 80%, 85%, 90%, 91%, 92%, 93%, 94%, 95%, 96%, 97%, 98%, 99%, or 100% identical to amino acids of 31-172 of SEQ ID NO: 513. In some embodiments, heteromultimers of the disclosure comprise at least one Cripto-1 polypeptide that is at least 70%, 75%, 80%, 85%, 90%, 91%, 92%, 93%, 94%, 95%, 96%, 97%, 98%, 99%, or 100% identical to amino acids of 63-188 of SEQ ID NO: 513. In some embodiments, heteromultimers of the disclosure comprise at least one Cripto-1 polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 15-66 (e.g., amino acid residues 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, or 66) of SEQ ID NO: 517, and ends at any one of amino acids 156-172 (e.g., amino acid residues 156, 157, 158, 159, 160, 161, 162, 163, 164, 165, 166, 167, 168, 169, 170, 171, or 172) of SEQ ID NO: 517. In some embodiments, heteromultimers of the disclosure comprise at least one Cripto-1 polypeptide that is at least 70%, 75%, 80%, 85%, 90%, 91%, 92%, 93%, 94%, 95%, 96%, 97%, 98%, 99%, or 100% identical to amino acids of 15-172 of SEQ ID NO: 517. In some embodiments, heteromultimers of the disclosure comprise at least one Cripto-1 polypeptide that is at least 70%, 75%, 80%, 85%, 90%, 91%, 92%, 93%, 94%, 95%, 96%, 97%, 98%, 99%, or 100% identical to amino acids of 47-172 of SEQ ID NO: 517. In some embodiments, heteromultimers of the disclosure comprise at least one Cripto-1 polypeptide that is at least 70%, 75%, 80%, 85%, 90%, 91%, 92%, 93%, 94%, 95%, 96%, 97%, 98%, 99%, or 100% identical to amino acids of 47-156 of SEQ ID NO: 517. In some embodiments, heteromultimers of the disclosure comprise at least one Cripto-1 polypeptide that is at least 70%, 75%, 80%, 85%, 90%, 91%, 92%, 93%, 94%, 95%, 96%, 97%, 98%, 99%, or 100% identical to amino acids of 66-165 of SEQ ID NO: 517. In some embodiments, heteromultimers of the disclosure comprise at least one Cripto-1 polypeptide that is at least 70%, 75%, 80%, 85%, 90%, 91%, 92%, 93%, 94%, 95%, 96%, 97%, 98%, 99%, or 100% identical to amino acids of 15-156 of SEQ ID NO: 517. In some embodiments, heteromultimers of the disclosure comprise at least one Cripto-1 polypeptide that is at least 70%, 75%, 80%, 85%, 90%, 91%, 92%, 93%, 94%, 95%, 96%, 97%, 98%, 99%, or 100% identical to amino acids of 66-172 of SEQ ID NO: 517. In some embodiments, heteromultimers of the disclosure comprise at least one Cripto-1 polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 31-82 (e.g., amino acid residues 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, or 82) of SEQ ID NO: 513, and ends at any one of amino acids 181-188 (e.g., amino acid residues 181, 182, 183, 184, 185, 185, 187, or 188) of SEQ ID NO: 513. In some embodiments, heteromultimers of the disclosure comprise at least one Cripto-1 polypeptide that is at least 70%, 75%, 80%, 85%, 90%, 91%, 92%, 93%, 94%, 95%, 96%, 97%, 98%, 99%, or 100% identical to amino acids of 31-188 of SEQ ID NO: 513. In some embodiments, heteromultimers of the disclosure comprise at least one Cripto-1 polypeptide that is at least 70%, 75%, 80%, 85%, 90%, 91%, 92%, 93%, 94%, 95%, 96%, 97%, 98%, 99%, or 100% identical to amino acids of 82-181 of SEQ ID NO: 513. In some embodiments, heteromultimers of the disclosure comprise at least one Cripto-1 polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 1-66 (e.g., amino acid residues 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, or 66) of SEQ ID NO: 517, and ends at any one of amino acids 165-172 (e.g., amino acid residues 165, 166, 167, 168, 169, 170, 171, or 172) of SEQ ID NO: 517. In some embodiments, heteromultimers of the disclosure comprise at least one Cripto-1 polypeptide that is at least 70%, 75%, 80%, 85%, 90%, 91%, 92%, 93%, 94%, 95%, 96%, 97%, 98%, 99%, or 100% identical to amino acids of 1-172 of SEQ ID NO: 517. In some embodiments, heteromultimers of the disclosure comprise at least one Cripto-1 polypeptide that is at least 70%, 75%, 80%, 85%, 90%, 91%, 92%, 93%, 94%, 95%, 96%, 97%, 98%, 99%, or 100% identical to amino acids of 66-165 of SEQ ID NO: 517. In some embodiments, heteromultimers of the disclosure comprise at least one Cripto-1 polypeptide that is at least 70%, 75%, 80%, 85%, 90%, 91%, 92%, 93%, 94%, 95%, 96%, 97%, 98%, 99%, or 100% identical to amino acids of 31-61 of SEQ ID NO: 513. In some embodiments, heteromultimers of the disclosure comprise at least one Cripto-1 polypeptide that is at least 70%, 75%, 80%, 85%, 90%, 91%, 92%, 93%, 94%, 95%, 96%, 97%, 98%, 99%, or 100% identical to amino acids of 63-161 of SEQ ID NO: 513. In some embodiments, heteromultimers of the disclosure comprise at least one Cripto-1 polypeptide that is at least 70%, 75%, 80%, 85%, 90%, 91%, 92%, 93%, 94%, 95%, 96%, 97%, 98%, 99%, or 100% identical to amino acids of 1-145 of SEQ ID NO: 517.

The term “Cryptic polypeptide” includes polypeptides comprising any naturally occurring Cryptic protein (encoded by CFC1 or one of its nonhuman orthologs) as well as any variants thereof (including mutants, fragments, fusions, and peptidomimetic forms) that retain a useful activity.

The human Cryptic isoform 1 precursor protein sequence (NCBI Ref Seq NP_115934.1) is as follows:

| (SEQ ID NO: 521) | |
|---|---|
| 1 | MTWRHHVRLL FTVSLALQII NLGNSYQREK HNGGREEVTK VATQKHRQSP LNWTSSHFGE |
| 61 | VTGSAEGWGP EEPLPYSRAF GEGASARPRC CRNGGTCVLG SFCVCPAHFT GRYCEHDQRR |
| 121 | SECGALEHGA WTLRACHLCR CIFGALHCLP LQTPDRCDPK DFLASHAHGP SAGGAPSLLL |
| 181 | LLPCALLHRL LRPDAPAHPR SLVPSVLQRE RRPCGRPGLG HRL |

The signal peptide is indicated by single underline.

A processed Cryptic isoform 1 polypeptide sequence is as follows:

(SEQ ID NO: 522)

YQREKHNGGREEVTKVATQKHRQSPLNWTSSHFGEVTGSAEGWGPEEPLP
YSRAFGEGASARPRCCRNGGTCVLGSFCVCPAHFTGRYCEHDQRRSECGA
LEHGAWTLRACHLCRCIFGALHCLPLQTPDRCDPKDFLASHAHG

A nucleic acid sequence encoding unprocessed human Cryptic isoform 1 precursor protein is shown below (SEQ ID NO: 523), corresponding to nucleotides 289-957 of NCBI Reference Sequence NM_032545.3. The signal sequence is underlined.

(SEQ ID NO: 523)

ATGACCTGGAGGCACCATGTCAGGCTTCTGTTTACGGTCAGTTTGGCATT
ACAGATCATCAATTTGGGAAACAGCTATCAAAGAGAGAAACATAACGGCG
GTAGAGAGGAAGTCACCAAGGTTGCCACTCAGAAGCACCGACAGTCACCG
CTCAACTGGACCTCCAGTCATTTCGGAGAGGTGACTGGGAGCGCCGAGGG
CTGGGGGCCGGAGGAGCCGCTCCCCTACTCCCGGGCTTTCGGAGAGGGTG
CGTCCGCGCGGCCGCGCTGCTGCAGGAACGGCGGTACCTGCGTGCTGGGC
AGCTTCTGCGTGTGCCCGGCCCACTTCACCGGCCGCTACTGCGAGCATGA
CCAGAGGCGCAGTGAATGCGGCGCCCTGGAGCACGGAGCCTGGACCCTCC
GCGCCTGCCACCTCTGCAGGTGCATCTTCGGGGCCCTGCACTGCCTCCCC
CTCCAGACGCCTGACCGCTGTGACCCGAAAGACTTCCTGGCCTCCCACGC
TCACGGGCCGAGCGCCGGGGGCGCGCCCAGCCTGCTACTCTTGCTGCCCT
GCGCACTCCTGCACCGCCTCCTGCGCCCGGATGCGCCCGCGCACCCTCGG
TCCCTGGTCCCTTCCGTCCTCCAGCGGGAGCGGCGCCCCTGCGGAAGGCC
GGGACTTGGGCATCGCCTT

A nucleic acid sequence encoding a processed human Cryptic isoform 1 is shown below (SEQ ID NO: 524):

(SEQ ID NO: 524)

TATCAAAGAGAGAAACATAACGGCGGTAGAGAGGAAGTCACCAAGGTTGC
CACTCAGAAGCACCGACAGTCACCGCTCAACTGGACCTCCAGTCATTTCG
GAGAGGTGACTGGGAGCGCCGAGGGCTGGGGGCCGGAGGAGCCGCTCCCC
TACTCCCGGGCTTTCGGAGAGGGTGCGTCCGCGCGGCCGCGCTGCTGCAG
GAACGGCGGTACCTGCGTGCTGGGCAGCTTCTGCGTGTGCCCGGCCCACT
TCACCGGCCGCTACTGCGAGCATGACCAGAGGCGCAGTGAATGCGGCGCC
CTGGAGCACGGAGCCTGGACCCTCCGCGCCTGCCACCTCTGCAGGTGCAT
CTTCGGGGCCCTGCACTGCCTCCCCCTCCAGACGCCTGACCGCTGTGACC
CGAAAGACTTCCTGGCCTCCCACGCTCACGGG

The human Cryptic isoform 2 precursor protein sequence (NCBI Ref Seq NP_001257349.1) is as follows:

| (SEQ ID NO: 525) | |
|---|---|
| 1 | MTWRHHVRLL FTVSLALQII NLGNSYQREK HNGGREEVTK VATQKHRQSP LNWTSSHFGE |
| 61 | VTGSAEGWGP EEPLPYSRAF GEVNAAPWST EPGPSAPATS AGASSGPCTA SPSRRLTAVT |
| 121 | RKTSWPPTLT GRAPGARPAC YSCCPAHSCT ASCARMRPRT LGPWSLPSSS GSGAPAEGRD |
| 181 | LGIAFNFLCC K |

The signal peptide is indicated by single underline.

A processed Cryptic isoform 2 polypeptide sequence is as follows:

(SEQ ID NO: 526)

YQREKHNGGREEVTKVATQKHRQSPLNWTSSHFGEVTGSAEGWGPEEPLP
YSRAFGEVNAAPWSTEPGPSAPATSAGASSGPCTASPSRRLTAVTRKTSW
PPTLTGRAPGARPACYSCCPAHSCTASCARMRPRTLGPWSLPSSSGSGAP
AEGRDLGIAFNFLCCK

A nucleic acid sequence encoding unprocessed human Cryptic isoform 2 precursor protein is shown below (SEQ ID NO: 527), corresponding to nucleotides 289-861 of NCBI Reference Sequence NM_001270420.1. The signal sequence is underlined.

(SEQ ID NO: 527)

ATGACCTGGAGGCACCATGTCAGGCTTCTGTTTACGGTCAGTTTGGCATT
ACAGATCATCAATTTGGGAAACAGCTATCAAAGAGAGAAACATAACGGCG
GTAGAGAGGAAGTCACCAAGGTTGCCACTCAGAAGCACCGACAGTCACCG
CTCAACTGGACCTCCAGTCATTTCGGAGAGGTGACTGGGAGCGCCGAGGG
CTGGGGGCCGGAGGAGCCGCTCCCCTACTCCCGGGCTTTCGGAGAGGTGA
ATGCGGCGCCCTGGAGCACGGAGCCTGGACCCTCCGCGCCTGCCACCTCT
GCAGGTGCATCTTCGGGGCCCTGCACTGCCTCCCCCTCCAGACGCCTGAC
CGCTGTGACCCGAAAGACTTCCTGGCCTCCCACGCTCACGGGCCGAGCGC
CGGGGGCGCGCCCAGCCTGCTACTCTTGCTGCCCTGCGCACTCCTGCACC
GCCTCCTGCGCCCGGATGCGCCCGCGCACCCTCGGTCCCTGGTCCCTTCC
GTCCTCCAGCGGGAGCGGCGCCCCTGCGGAAGGCCGGGACTTGGGCATCG
CCTTTAATTTTCTATGTTGTAAA

A nucleic acid sequence encoding processed Cryptic isoform 2 is shown below (SEQ ID NO: 528):

(SEQ ID NO: 528)

TATCAAAGAGAGAAACATAACGGCGGTAGAGAGGAAGTCACCAAGGTTGC
CACTCAGAAGCACCGACAGTCACCGCTCAACTGGACCTCCAGTCATTTCG
GAGAGGTGACTGGGAGCGCCGAGGGCTGGGGGCCGGAGGAGCCGCTCCCC
TACTCCCGGGCTTTCGGAGAGGTGAATGCGGCGCCCTGGAGCACGGAGCC
TGGACCCTCCGCGCCTGCCACCTCTGCAGGTGCATCTTCGGGGCCCTGCA
CTGCCTCCCCCTCCAGACGCCTGACCGCTGTGACCCGAAAGACTTCCTGG
CCTCCCACGCTCACGGGCCGAGCGCCGGGGGCGCGCCCAGCCTGCTACTC
TTGCTGCCCTGCGCACTCCTGCACCGCCTCCTGCGCCCGGATGCGCCCGC
GCACCCTCGGTCCCTGGTCCCTTCCGTCCTCCAGCGGGAGCGGCGCCCCT
GCGGAAGGCCGGGACTTGGGCATCGCCTTTAATTTTCTATGTTGTAAA

The human Cryptic isoform 3 precursor protein sequence (NCBI Ref Seq NP_001257350.1) is as follows:

| (SEQ ID NO: 529) | |
|---|---|
| 1 | MTWRHHVRLL FTVSLALQII NLGNSYQREK HNGGREEVTK VATQKHRQSP LNWTSSHFGE |
| 61 | VTGSAEGWGP EEPLPYSRAF GEDPKDFLAS HAHGPSAGGA PSLLLLLPCA LLHRLLRPDA |
| 121 | PAHPRSLVPS VLQRERRPCG RPGLGHRL |

The signal peptide is indicated by single underline.

A processed Cryptic isoform 3 polypeptide sequence is as follows:

(SEQ ID NO: 530)

YQREKHNGGREEVTKVATQKHRQSPLNWTSSHFGEVTGSAEGWGPEEPLP
YSRAFGEDPKDFLASHAHG

A nucleic acid sequence encoding unprocessed human Cryptic isoform 3 precursor protein is shown below (SEQ ID NO: 531), corresponding to nucleotides 289-732 of NCBI Reference Sequence NM_001270421.1. The signal sequence is underlined.

(SEQ ID NO: 531)

ATGACCTGGAGGCACCATGTCAGGCTTCTGTTTACGGTCAGTTTGGCATT
ACAGATCATCAATTTGGGAAACAGCTATCAAAGAGAGAAACATAACGGCG
GTAGAGAGGAAGTCACCAAGGTTGCCACTCAGAAGCACCGACAGTCACCG
CTCAACTGGACCTCCAGTCATTTCGGAGAGGTGACTGGGAGCGCCGAGGG
CTGGGGGCCGGAGGAGCCGCTCCCCTACTCCCGGGCTTTCGGAGAGGACC
CGAAAGACTTCCTGGCCTCCCACGCTCACGGGCCGAGCGCCGGGGGCGCG
CCCAGCCTGCTACTCTTGCTGCCCTGCGCACTCCTGCACCGCCTCCTGCG
CCCGGATGCGCCCGCGCACCCTCGGTCCCTGGTCCCTTCCGTCCTCCAGC
GGGAGCGGCGCCCCTGCGGAAGGCCGGGACTTGGGCATCGCCTT

A nucleic acid sequence encoding a processed Cryptic isoform 3 is shown below (SEQ ID NO: 532):

(SEQ ID NO: 532)

TATCAAAGAGAGAAACATAACGGCGGTAGAGAGGAAGTCACCAAGGTTGC
CACTCAGAAGCACCGACAGTCACCGCTCAACTGGACCTCCAGTCATTTCG
GAGAGGTGACTGGGAGCGCCGAGGGCTGGGGGCCGGAGGAGCCGCTCCCC
TACTCCCGGGCTTTCGGAGAGGACCCGAAAGACTTCCTGGCCTCCCACGC
TCACGGG

In certain embodiments, the disclosure relates to heteromultimers that comprise at least one Cryptic polypeptide, which includes fragments, functional variants, and modified forms thereof. Preferably, Cryptic polypeptides for use in accordance with the disclosure (e.g., heteromultimers comprising a Cryptic polypeptide and uses thereof) are soluble (e.g., an extracellular domain of Cryptic). In other preferred embodiments, Cryptic polypeptides for use in accordance with the disclosure bind to and/or inhibit (antagonize) activity (e.g., Smad signaling) of one or more TGF-beta superfamily ligands. In some embodiments, heteromultimers of the disclosure comprise at least one Cryptic polypeptide that is at least 70%, 75%, 80%, 85%, 90%, 91%, 92%, 93%, 94%, 95%, 96%, 97%, 98%, 99%, or 100% identical to the amino acid sequence of SEQ ID NOs: 521, 522, 525, 526, 529, or 530. In some embodiments, heteromultimers of the disclosure comprise at least one Cryptic polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 26-90 (e.g., amino acid residues 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90) of SEQ ID NO: 521, and ends at any one of amino acids 157-223 (e.g., amino acid residues 157, 158, 159, 160, 161, 162, 163, 164, 165, 166, 167, 168, 169, 170, 171, 172, 173, 174, 175, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 126, 217, 218, 219, 220, 221, 222, or 223) of SEQ ID NO: 521. In some embodiments, heteromultimers of the disclosure comprise at least one Cryptic polypeptide that is at least 70%, 75%, 80%, 85%, 90%, 91%, 92%, 93%, 94%, 95%, 96%, 97%, 98%, 99%, or 100% identical to amino acids of 26-223 of SEQ ID NO: 521. In some embodiments, heteromultimers of the disclosure comprise at least one Cryptic polypeptide that is at least 70%, 75%, 80%, 85%, 90%, 91%, 92%, 93%, 94%, 95%, 96%, 97%, 98%, 99%, or 100% identical to amino acids of 26-157 of SEQ ID NO: 521. In some embodiments, heteromultimers of the disclosure comprise at least one Cryptic polypeptide that is at least 70%, 75%, 80%, 85%, 90%, 91%, 92%, 93%, 94%, 95%, 96%, 97%, 98%, 99%, or 100% identical to amino acids of 90-157 of SEQ ID NO: 521. In some embodiments, heteromultimers of the disclosure comprise at least one Cryptic polypeptide that is at least 70%, 75%, 80%, 85%, 90%, 91%, 92%, 93%, 94%, 95%, 96%, 97%, 98%, 99%, or 100% identical to amino acids of 26-169 of SEQ ID NO: 521. In some embodiments, heteromultimers of the disclosure comprise at least one Cryptic polypeptide that is at least 70%, 75%, 80%, 85%, 90%, 91%, 92%, 93%, 94%, 95%, 96%, 97%, 98%, 99%, or 100% identical to amino acids of 90-169 of SEQ ID NO: 521. In some embodiments, heteromultimers of the disclosure comprise at least one Cryptic polypeptide that is at least 70%, 75%, 80%, 85%, 90%, 91%, 92%, 93%, 94%, 95%, 96%, 97%, 98%, 99%, or 100% identical to amino acids of 90-223 of SEQ ID NO: 521. In some embodiments, heteromultimers of the disclosure comprise at least one Cryptic polypeptide that is at least 70%, 75%, 80%, 85%, 90%, 91%, 92%, 93%, 94%, 95%, 96%, 97%, 98%, 99%, or 100% identical to amino acids of 26-82 of SEQ ID NO: 521. In some embodiments, heteromultimers of the disclosure comprise at least one Cryptic polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 26-30 (e.g., amino acid residues 26, 27, 28, 29, or 30) of SEQ ID NO: 525, and ends at any one of amino acids 82-191 (e.g., amino acid residues 82, 83, 84, 85, 86, 57, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 178, 179, 180, 181, 182, 183, 184, 185, 186, 187, 188, 189, 190, or 191) of SEQ ID NO: 525. In some embodiments, heteromultimers of the disclosure comprise at least one Cryptic polypeptide that is at least 70%, 75%, 80%, 85%, 90%, 91%, 92%, 93%, 94%, 95%, 96%, 97%, 98%, 99%, or 100% identical to amino acids of 26-82 of SEQ ID NO: 525. In some embodiments, heteromultimers of the disclosure comprise at least one Cryptic polypeptide that is at least 70%, 75%, 80%, 85%, 90%, 91%, 92%, 93%, 94%, 95%, 96%, 97%, 98%, 99%, or 100% identical to amino acids of 26-191 of SEQ ID NO: 525. In some embodiments, heteromultimers of the disclosure comprise at least one Cryptic polypeptide that is at least 70%, 75%, 80%, 85%, 90%, 91%, 92%, 93%, 94%, 95%, 96%, 97%, 98%, 99%, or 100% identical to amino acids of 30-82 of SEQ ID NO: 525. In some embodiments, heteromultimers of the disclosure comprise at least one Cryptic polypeptide that is at least 70%, 75%, 80%, 85%, 90%, 91%, 92%, 93%, 94%, 95%, 96%, 97%, 98%, 99%, or 100% identical to amino acids of 30-191 of SEQ ID NO: 525. In some embodiments, heteromultimers of the disclosure comprise at least one Cryptic polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 26-30 (e.g., amino acid residues 26, 27, 28, 29, or 30) of SEQ ID NO: 529, and ends at any one of amino acids 82-148 (e.g., amino acid residues 82, 83, 84, 85, 86, 57, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, or 148) of SEQ ID NO: 529. In some embodiments, heteromultimers of the disclosure comprise at least one Cryptic polypeptide that is at least 70%, 75%, 80%, 85%, 90%, 91%, 92%, 93%, 94%, 95%, 96%, 97%, 98%, 99%, or 100% identical to amino acids of 26-148 of SEQ ID NO: 529. In some embodiments, heteromultimers of the disclosure comprise at least one Cryptic polypeptide that is at least 70%, 75%, 80%, 85%, 90%, 91%, 92%, 93%, 94%, 95%, 96%, 97%, 98%, 99%, or 100% identical to amino acids of 26-82 of SEQ ID NO: 529. In some embodiments, heteromultimers of the disclosure comprise at least one Cryptic polypeptide that is at least 70%, 75%, 80%, 85%, 90%, 91%, 92%, 93%, 94%, 95%, 96%, 97%, 98%, 99%, or 100% identical to amino acids of 30-148 of SEQ ID NO: 529. In some embodiments, heteromultimers of the disclosure comprise at least one Cryptic polypeptide that is at least 70%, 75%, 80%, 85%, 90%, 91%, 92%, 93%, 94%, 95%, 96%, 97%, 98%, 99%, or 100% identical to amino acids of 30-82 of SEQ ID NO: 529. In some embodiments, heteromultimers of the disclosure comprise at least one Cryptic polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 26-90 (e.g., amino acid residues 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90) of SEQ ID NO: 521, and ends at any one of amino acids 214-223 (e.g., amino acid residues 214, 215, 126, 217, 218, 219, 220, 221, 222, or 223) of SEQ ID NO: 521. In some embodiments, heteromultimers of the disclosure comprise at least one Cryptic polypeptide that is at least 70%, 75%, 80%, 85%, 90%, 91%, 92%, 93%, 94%, 95%, 96%, 97%, 98%, 99%, or 100% identical to amino acids of 26-223 of SEQ ID NO: 521. In some embodiments, heteromultimers of the disclosure comprise at least one Cryptic polypeptide that is at least 70%, 75%, 80%, 85%, 90%, 91%, 92%, 93%, 94%, 95%, 96%, 97%, 98%, 99%, or 100% identical to amino acids of 109-223 of SEQ ID NO: 521. In some embodiments, heteromultimers of the disclosure comprise at least one Cryptic polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 26-108 (e.g., amino acid residues 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, or 108) of SEQ ID NO: 525, and ends at any one of amino acids 189-191 (e.g., amino acid residues 189, 190, or 191) of SEQ ID NO: 525. In some embodiments, heteromultimers of the disclosure comprise at least one Cryptic polypeptide that is at least 70%, 75%, 80%, 85%, 90%, 91%, 92%, 93%, 94%, 95%, 96%, 97%, 98%, 99%, or 100% identical to amino acids of 26-191 of SEQ ID NO: 525. In some embodiments, heteromultimers of the disclosure comprise at least one Cryptic polypeptide that is at least 70%, 75%, 80%, 85%, 90%, 91%, 92%, 93%, 94%, 95%, 96%, 97%, 98%, 99%, or 100% identical to amino acids of 108-189 of SEQ ID NO: 525. In some embodiments, heteromultimers of the disclosure comprise at least one Cryptic polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 26-109 (e.g., amino acid residues 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108 or 109) of SEQ ID NO: 529, and ends at any one of amino acids 139-148 (e.g., amino acid residues 139, 140, 141, 142, 143, 144, 145, 146, 147, or 148) of SEQ ID NO: 529. In some embodiments, heteromultimers of the disclosure comprise at least one Cryptic polypeptide that is at least 70%, 75%, 80%, 85%, 90%, 91%, 92%, 93%, 94%, 95%, 96%, 97%, 98%, 99%, or 100% identical to amino acids of 26-148 of SEQ ID NO: 529. In some embodiments, heteromultimers of the disclosure comprise at least one Cryptic polypeptide that is at least 70%, 75%, 80%, 85%, 90%, 91%, 92%, 93%, 94%, 95%, 96%, 97%, 98%, 99%, or 100% identical to amino acids of 109-139 of SEQ ID NO: 529. In some embodiments, heteromultimers of the disclosure comprise at least one Cryptic polypeptide that is at least 70%, 75%, 80%, 85%, 90%, 91%, 92%, 93%, 94%, 95%, 96%, 97%, 98%, 99%, or 100% identical to amino acids of 26-94 of SEQ ID NO: 529.

The term “Cryptic family protein 1B polypeptide” includes polypeptides comprising any naturally occurring Cryptic family protein 1B protein (encoded by CFC1B or one of its nonhuman orthologs) as well as any variants thereof (including mutants, fragments, fusions, and peptidomimetic forms) that retain a useful activity.

The human Cryptic family protein 1B precursor protein sequence (NCBI Ref Seq NP_001072998.1) is as follows:

| (SEQ ID NO: 533) | |
|---|---|
| 1 | MTWRHHVRLL FTVSLALQII NLGNSYQREK HNGGREEVTK VATQKHRQSP LNWTSSHFGE |
| 61 | VTGSAEGWGP EEPLPYSWAF GEGASARPRC CRNGGTCVLG SFCVCPAHFT GRYCEHDQRR |
| 121 | SECGALEHGA WTLRACHLCR CIFGALHCLP LQTPDRCDPK DFLASHAHGP SAGGAPSLLL |
| 181 | LLPCALLHRL LRPDAPAHPR SLVPSVLQRE RRPCGRPGLG HRL |

The signal peptide is indicated by single underline.

A processed Cryptic family protein 1B polypeptide sequence is as follows:

(SEQ ID NO: 534)

YQREKHNGGREEVTKVATQKHRQSPLNWTSSHFGEVTGSAEGWGPEEPLP
YSWAFGEGASARPRCCRNGGTCVLGSFCVCPAHFTGRYCEHDQRRSECG
ALEHGAWTLRACHLCRCIFGALHCLPLQTPDRCDPKDFLASHAHG

A nucleic acid sequence encoding unprocessed human Cryptic family protein 1B precursor protein is shown below (SEQ ID NO: 535), corresponding to nucleotides 392-1060 of NCBI Reference Sequence NM_001079530.1. The signal sequence is underlined.

(SEQ ID NO: 535)

ATGACCTGGAGGCACCATGTCAGGCTTCTGTTTACGGTCAGTTTGGCATT
ACAGATCATCAATTTGGGAAACAGCTATCAAAGAGAGAAACATAACGGCG
GTAGAGAGGAAGTCACCAAGGTTGCCACTCAGAAGCACCGACAGTCACCG
CTCAACTGGACCTCCAGTCATTTCGGAGAGGTGACTGGGAGCGCCGAGGG
CTGGGGGCCGGAGGAGCCGCTCCCATACTCCTGGGCTTTCGGAGAGGGTG
CGTCCGCGCGGCCGCGCTGCTGCAGGAACGGCGGTACCTGCGTGCTGGGC
AGCTTCTGCGTGTGCCCGGCCCACTTCACCGGCCGCTACTGCGAGCATGA
CCAGAGGCGCAGTGAATGCGGCGCCCTGGAGCACGGAGCCTGGACCCTCC
GCGCCTGCCACCTCTGCAGGTGCATCTTCGGGGCCCTGCACTGCCTCCCC
CTCCAGACGCCTGACCGCTGTGACCCGAAAGACTTCCTGGCCTCCCACGC
TCACGGGCCGAGCGCCGGGGGCGCGCCCAGCCTGCTACTCTTGCTGCCCT
GCGCACTCCTGCACCGCCTCCTGCGCCCGGATGCGCCCGCGCACCCTCGG
TCCCTGGTCCCTTCCGTCCTCCAGCGGGAGCGGCGCCCCTGCGGAAGGCC
GGGACTTGGGCATCGCCTT

A nucleic acid sequence encoding a processed Cryptic family protein 1B is shown below (SEQ ID NO: 536):

(SEQ ID NO: 536)

TATCAAAGAGAGAAACATAACGGCGGTAGAGAGGAAGTCACCAAGGTTGC
CACTCAGAAGCACCGACAGTCACCGCTCAACTGGACCTCCAGTCATTTCG
GAGAGGTGACTGGGAGCGCCGAGGGCTGGGGGCCGGAGGAGCCGCTCCCA
TACTCCTGGGCTTTCGGAGAGGGTGCGTCCGCGCGGCCGCGCTGCTGCAG
GAACGGCGGTACCTGCGTGCTGGGCAGCTTCTGCGTGTGCCCGGCCCACT
TCACCGGCCGCTACTGCGAGCATGACCAGAGGCGCAGTGAATGCGGCGCC
CTGGAGCACGGAGCCTGGACCCTCCGCGCCTGCCACCTCTGCAGGTGCAT
CTTCGGGGCCCTGCACTGCCTCCCCCTCCAGACGCCTGACCGCTGTGACC
CGAAAGACTTCCTGGCCTCCCACGCTCACGGG

In certain embodiments, the disclosure relates to heteromultimers that comprise at least one Cryptic family protein 1B polypeptide, which includes fragments, functional variants, and modified forms thereof. Preferably, Cryptic family protein 1B polypeptides for use in accordance with the disclosure (e.g., heteromultimers comprising a Cryptic family protein 1B polypeptide and uses thereof) are soluble (e.g., an extracellular domain of Cryptic family protein 1B). In other preferred embodiments, Cryptic family protein 1B polypeptides for use in accordance with the disclosure bind to and/or inhibit (antagonize) activity (e.g., Smad signaling) of one or more TGF-beta superfamily ligands. In some embodiments, heteromultimers of the disclosure comprise at least one Cryptic family protein 1B polypeptide that is at least 70%, 75%, 80%, 85%, 90%, 91%, 92%, 93%, 94%, 95%, 96%, 97%, 98%, 99%, or 100% identical to the amino acid sequence of SEQ ID NOs: 533 or 534. In some embodiments, heteromultimers of the disclosure comprise at least one Cryptic family protein 1B polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 26-30 (e.g., amino acid residues 26, 27, 28, 29, or 30) of SEQ ID NO: 533, and ends at any one of amino acids 82-223 (e.g., amino acid residues 82, 83, 84, 85, 86, 57, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 126, 217, 218, 219, 220, 221, 222, or 223) of SEQ ID NO: 533. In some embodiments, heteromultimers of the disclosure comprise at least one Cryptic family protein 1B polypeptide that is at least 70%, 75%, 80%, 85%, 90%, 91%, 92%, 93%, 94%, 95%, 96%, 97%, 98%, 99%, or 100% identical to amino acids of 26-223 of SEQ ID NO: 533. In some embodiments, heteromultimers of the disclosure comprise at least one Cryptic family protein 1B polypeptide that is at least 70%, 75%, 80%, 85%, 90%, 91%, 92%, 93%, 94%, 95%, 96%, 97%, 98%, 99%, or 100% identical to amino acids of 26-82 of SEQ ID NO: 533. In some embodiments, heteromultimers of the disclosure comprise at least one Cryptic family protein 1B polypeptide that is at least 70%, 75%, 80%, 85%, 90%, 91%, 92%, 93%, 94%, 95%, 96%, 97%, 98%, 99%, or 100% identical to amino acids of 30-82 of SEQ ID NO: 533. In some embodiments, heteromultimers of the disclosure comprise at least one Cryptic family protein 1B polypeptide that is at least 70%, 75%, 80%, 85%, 90%, 91%, 92%, 93%, 94%, 95%, 96%, 97%, 98%, 99%, or 100% identical to amino acids of 30-223 of SEQ ID NO: 533. In some embodiments, heteromultimers of the disclosure comprise at least one Cryptic family protein 1B polypeptide that is at least 70%, 75%, 80%, 85%, 90%, 91%, 92%, 93%, 94%, 95%, 96%, 97%, 98%, 99%, or 100% identical to amino acids of 26-169 of SEQ ID NO: 533. In some embodiments, heteromultimers of the disclosure comprise at least one Cryptic family protein 1B polypeptide that is at least 70%, 75%, 80%, 85%, 90%, 91%, 92%, 93%, 94%, 95%, 96%, 97%, 98%, 99%, or 100% identical to amino acids of 30-169 of SEQ ID NO: 533. In some embodiments, heteromultimers of the disclosure comprise at least one Cryptic family protein 1B polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 26-90 (e.g., amino acid residues 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90) of SEQ ID NO: 533, and ends at any one of amino acids 214-223 (e.g., amino acid residues 214, 215, 126, 217, 218, 219, 220, 221, 222, or 223) of SEQ ID NO: 533. In some embodiments, heteromultimers of the disclosure comprise at least one Cryptic family protein 1B polypeptide that is at least 70%, 75%, 80%, 85%, 90%, 91%, 92%, 93%, 94%, 95%, 96%, 97%, 98%, 99%, or 100% identical to amino acids of 26-223 of SEQ ID NO: 533. In some embodiments, heteromultimers of the disclosure comprise at least one Cryptic family protein 1B polypeptide that is at least 70%, 75%, 80%, 85%, 90%, 91%, 92%, 93%, 94%, 95%, 96%, 97%, 98%, 99%, or 100% identical to amino acids of 90-214 of SEQ ID NO: 533.

The term “CRIM1 polypeptide” includes polypeptides comprising any naturally occurring polypeptide of a CRIM1 protein (encoded by CRIM1 or one of its nonhuman orthologs) as well as any variants thereof (including mutants, fragments, fusions, and peptidomimetic forms) that retain a useful activity.

The human CRIM1 precursor protein sequence (NCBI Ref Seq NP_057525.1) is as follows:

| (SEQ ID NO: 537) | |
|---|---|
| 1 | MYLVAGDRGL AGCGHLLVSL LGLLLLLARS GTRALVCLPC DESKCEEPRN CPGSIVQGVC |
| 61 | GCCYTCASQR NESCGGTFGI YGTCDRGLRC VIRPPLNGDS LTEYEAGVCE DENWTDDQLL |
| 121 | GFKPCNENLI AGCNIINGKC ECNTIRTCSN PFEFPSQDMC LSALKRIEEE KPDCSKARCE |
| 181 | VQFSPRCPED SVLIEGYAPP GECCPLPSRC VCNPAGCLRK VCQPGNLNIL VSKASGKPGE |
| 241 | CCDLYECKPV FGVDCRTVEC PPVQQTACPP DSYETQVRLT ADGCCTLPTR CECLSGLCGF |
| 301 | PVCEVGSTPR IVSRGDGTPG KCCDVFECVN DTKPACVFNN VEYYDGDMFR MDNCRFCRCQ |
| 361 | GGVAICFTAQ CGEINCERYY VPEGECCPVC EDPVYPFNNP AGCYANGLIL AHGDRWREDD |
| 421 | CTFCQCVNGE RHCVATVCGQ TCTNPVKVPG ECCPVCEEPT IITVDPPACG ELSNCTLTGK |
| 481 | DCINGFKRDH NGCRTCQCIN TEELCSERKQ GCTLNCPFGF LTDAQNCEIC ECRPRPKKCR |
| 541 | PIICDKYCPL GLLKNKHGCD ICRCKKCPEL SCSKICPLGF QQDSHGCLIC KCREASASAG |
| 601 | PPILSGTCLT VDGHHHKNEE SWHDGCRECY CLNGREMCAL ITCPVPACGN PTIHPGQCCP |
| 661 | SCADDFVVQK PELSTPSICH APGGEYFVEG ETWNIDSCTQ CTCHSGRVLC ETEVCPPLLC |
| 721 | QNPSRTQDSC CPQCTDQPFR PSLSRNNSVP NYCKNDEGDI FLAAESWKPD VCTSCICIDS |
| 781 | VISCFSESCP SVSCERPVLR KGQCCPYCIE DTIPKKVVCH FSGKAYADEE RWDLDSCTHC |
| 841 | YCLQGQTLCS TVSCPPLPCV EPINVEGSCC PMCPEMYVPE PTNIPIEKTN HRGEVDLEVP |
| 901 | 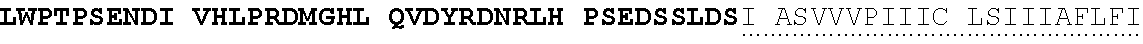 |
| 961 | NQKKQWIPLL CWYRTPTKPS SLNNQLVSVD CKKGTRVQVD SSQRMLRIAE PDARFSGFYS |
| 1021 | MQKQNHLQAD NFYQTV |

The signal peptide is indicated by a single underline, the extracellular domain is indicated by bold, and the transmembrane domain is indicated by dotted underline.

A mature CRIM1 sequence is as follows:

(SEQ ID NO: 538)

LVCLPCDESKCEEPRNCPGSIVQGVCGCCYTCASQRNESCGGTFGIYGTC
DRGLRCVIRPPLNGDSLTEYEAGVCEDENWTDDQLLGFKPCNENLIAGCN
IINGKCECNTIRTCSNPFEFPSQDMCLSALKRIEEEKPDCSKARCEVQFS
PRCPEDSVLIEGYAPPGECCPLPSRCVCNPAGCLRKVCQPGNLNILVSKA
SGKPGECCDLYECKPVFGVDCRTVECPPVQQTACPPDSYETQVRLTADGC
CTLPTCECLSGLCGFPVCEVGSTPRIVSRGDGTPGKCCDVFECVNDTKPA
CVFNNVEYYDGDMFRMDNCRFCRCQGGVAICFTAQCGEINCERYYVPEGE
CCPVCEDPVYPFNNPAGCYANGLILAHGDRWREDDCTFCQCVNGERHCVA
TVCGQTCTNPVKVPGECCPVCEEPTIITVDPPACGELSNCTLTGKDCING
FKRDHNGCRTCQCINTEELCSERKQGCTLNCPFGFLTDAQNCEICECRPR
PKKCRPIICDKYCPLGLLKNKHGCDICRCKKCPELSCSKICPLGFQQDSH
GCLICKCREASASAGPPILSGTCLTVDGHHHKNEESWHDGCRECYCLNGR
EMCALITCPVPACGNPTIHPGQCCPSCADDFVVQKPELSTPSICHAPGGE
YFVEGETWNIDSCTQCTCHSGRVLCETEVCPPLLCQNPSRTQDSCCPQCT
DQPFRPSLSRNNSVPNYCKNDEGDIFLAAESWKPDVCTSCICIDSVISCF
SESCPSVSCERPVLRKGQCCPYCIEDTIPKKVVCHFSGKAYADEERWDLD
SCTHCYCLQGQTLCSTVSCPPLPCVEPINVEGSCCPMCPEMYVPEPTNIP
IEKTNHRGEVDLEVPLWPTPSENDIVHLPRDMGHLQVDYRDNRLHPSEDS
SLDS

A nucleic acid sequence encoding unprocessed human CRIM1 precursor protein is shown below (SEQ ID NO: 539), corresponding to nucleotides 67-3174 of NCBI Reference Sequence NM_016441.2. The signal sequence is indicated by solid underline and the transmembrane region by dotted underline.

(SEQ ID NO: 539)
ATGTACTTGGTGGCGGGGGACAGGGGGTTGGCCGGCTGCGGGCACCTCCTGGTCTCGCTGCTGGGGCTGCTGCTG
CTGCTGGCGCGCTCCGGCACCCGGGCGCTGGTCTGCCTGCCCTGTGACGAGTCCAAGTGCGAGGAGCCCAGGAAC
TGCCCGGGGAGCATCGTGCAGGGCGTCTGCGGCTGCTGCTACACGTGCGCCAGCCAGAGGAACGAGAGCTGCGGC
GGCACCTTCGGGATTTACGGAACCTGCGACCGGGGGCTGCGTTGTGTCATCCGCCCCCCGCTCAATGGCGACTCC
CTCACCGAGTACGAAGCGGGCGTTTGCGAAGATGAGAACTGGACTGATGACCAACTGCTTGGTTTTAAACCATGC
AATGAAAACCTTATTGCTGGCTGCAATATAATCAATGGGAAATGTGAATGTAACACCATTCGAACCTGCAGCAAT
CCCTTTGAGTTTCCAAGTCAGGATATGTGCCTTTCAGCTTTAAAGAGAATTGAAGAAGAGAAGCCAGATTGCTCC
AAGGCCCGCTGTGAAGTCCAGTTCTCTCCACGTTGTCCTGAAGATTCTGTTCTGATCGAGGGTTATGCTCCTCCT
GGGGAGTGCTGTCCCTTACCCAGCCGCTGCGTGTGCAACCCCGCAGGCTGTCTGCGCAAAGTCTGCCAGCCGGGA
AACCTGAACATACTAGTGTCAAAAGCCTCAGGGAAGCCGGGAGAGTGCTGTGACCTCTATGAGTGCAAACCAGTT
TTCGGCGTGGACTGCAGGACTGTGGAATGCCCTCCTGTTCAGCAGACCGCGTGTCCCCCGGACAGCTATGAAACT
CAAGTCAGACTAACTGCAGATGGTTGCTGTACTTTGCCAACAAGATGCGAGTGTCTCTCTGGCTTATGTGGTTTC
CCCGTGTGTGAGGTGGGATCCACTCCCCGCATAGTCTCTCGTGGCGATGGGACACCTGGAAAGTGCTGTGATGTC
TTTGAATGTGTTAATGATACAAAGCCAGCCTGCGTATTTAACAATGTGGAATATTATGATGGAGACATGTTTCGA
ATGGACAACTGTCGGTTCTGTCGATGCCAAGGGGGCGTTGCCATCTGCTTCACCGCCCAGTGTGGTGAGATAAAC
TGCGAGAGGTACTACGTGCCCGAAGGAGAGTGCTGCCCAGTGTGTGAAGATCCAGTGTATCCTTTTAATAATCCC
GCTGGCTGCTATGCCAATGGCCTGATCCTTGCCCACGGAGACCGGTGGCGGGAAGACGACTGCACATTCTGCCAG
TGCGTCAACGGTGAACGCCACTGCGTTGCGACCGTCTGCGGACAGACCTGCACAAACCCTGTGAAAGTGCCTGGG
GAGTGTTGCCCTGTGTGCGAAGAACCAACCATCATCACAGTTGATCCACCTGCATGTGGGGAGTTATCAAACTGC
ACTCTGACAGGGAAGGACTGCATTAATGGTTTCAAACGCGATCACAATGGTTGTCGGACCTGTCAGTGCATAAAC
ACCGAGGAACTATGTTCAGAACGTAAACAAGGCTGCACCTTGAACTGTCCCTTCGGTTTCCTTACTGATGCCCAA
AACTGTGAGATCTGTGAGTGCCGCCCAAGGCCCAAGAAGTGCAGACCCATAATCTGTGACAAGTATTGTCCACTT
GGATTGCTGAAGAATAAGCACGGCTGTGACATCTGTCGCTGTAAGAAATGTCCAGAGCTCTCATGCAGTAAGATC
TGCCCCTTGGGTTTCCAGCAGGACAGTCACGGCTGTCTTATCTGCAAGTGCAGAGAGGCCTCTGCTTCAGCTGGG
CCACCCATCCTGTCGGGCACTTGTCTCACCGTGGATGGTCATCATCATAAAAATGAGGAGAGCTGGCACGATGGG
TGCCGGGAATGCTACTGTCTCAATGGACGGGAAATGTGTGCCCTGATCACCTGCCCGGTGCCTGCCTGTGGCAAC
CCCACCATTCACCCTGGACAGTGCTGCCCATCATGTGCAGATGACTTTGTGGTGCAGAAGCCAGAGCTCAGTACT
CCCTCCATTTGCCACGCCCCTGGAGGAGAATACTTTGTGGAAGGAGAAACGTGGAACATTGACTCCTGTACTCAG
TGCACCTGCCACAGCGGACGGGTGCTGTGTGAGACAGAGGTGTGCCCACCGCTGCTCTGCCAGAACCCCTCACGC
ACCCAGGATTCCTGCTGCCCACAGTGTACAGATCAACCTTTTCGGCCTTCCTTGTCCCGCAATAACAGCGTACCT
AATTACTGCAAAAATGATGAAGGGGATATATTCCTGGCAGCTGAGTCCTGGAAGCCTGACGTTTGTACCAGCTGC
ATCTGCATTGATAGCGTAATTAGCTGTTTCTCTGAGTCCTGCCCTTCTGTATCCTGTGAAAGACCTGTCTTGAGA
AAAGGCCAGTGTTGTCCCTACTGCATAGAAGACACAATTCCAAAGAAGGTGGTGTGCCACTTCAGTGGGAAGGCC
TATGCCGACGAGGAGCGGTGGGACCTTGACAGCTGCACCCACTGCTACTGCCTGCAGGGCCAGACCCTCTGCTCG
ACCGTCAGCTGCCCCCCTCTGCCCTGTGTTGAGCCCATCAACGTGGAAGGAAGTTGCTGCCCAATGTGTCCAGAA
ATGTATGTCCCAGAACCAACCAATATACCCATTGAGAAGACAAACCATCGAGGAGAGGTTGACCTGGAGGTTCCC
CTGTGGCCCACGCCTAGTGAAAATGATATCGTCCATCTCCCTAGAGATATGGGTCACCTCCAGGTAGATTACAGA

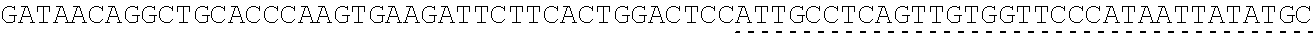

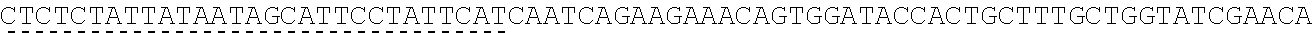

CCAACTAAGCCTTCTTCCTTAAATAATCAGCTAGTATCTGTGGACTGCAAGAAAGGAACCAGAGTCCAGGTGGAC
AGTTCCCAGAGAATGCTAAGAATTGCAGAACCAGATGCAAGATTCAGTGGCTTCTACAGCATGCAAAAACAGAAC
CATCTACAGGCAGACAATTTCTACCAAACAGTG

A nucleic acid sequence encoding processed extracellular human CRIM1 is shown below (SEQ ID NO: 540):

(SEQ ID NO: 540)

CTGGTCTGCCTGCCCTGTGACGAGTCCAAGTGCGAGGAGCCCAGGAACTG
CCCGGGGAGCATCGTGCAGGGCGTCTGCGGCTGCTGCTACACGTGCGCCA
GCCAGAGGAACGAGAGCTGCGGCGGCACCTTCGGGATTTACGGAACCTGC
GACCGGGGGCTGCGTTGTGTCATCCGCCCCCCGCTCAATGGCGACTCCCT
CACCGAGTACGAAGCGGGCGTTTGCGAAGATGAGAACTGGACTGATGACC
AACTGCTTGGTTTTAAACCATGCAATGAAAACCTTATTGCTGGCTGCAAT
ATAATCAATGGGAAATGTGAATGTAACACCATTCGAACCTGCAGCAATCC
CTTTGAGTTTCCAAGTCAGGATATGTGCCTTTCAGCTTTAAAGAGAATTG
AAGAAGAGAAGCCAGATTGCTCCAAGGCCCGCTGTGAAGTCCAGTTCTCT
CCACGTTGTCCTGAAGATTCTGTTCTGATCGAGGGTTATGCTCCTCCTGG
GGAGTGCTGTCCCTTACCCAGCCGCTGCGTGTGCAACCCCGCAGGCTGTC
TGCGCAAAGTCTGCCAGCCGGGAAACCTGAACATACTAGTGTCAAAAGCC
TCAGGGAAGCCGGGAGAGTGCTGTGACCTCTATGAGTGCAAACCAGTTTT
CGGCGTGGACTGCAGGACTGTGGAATGCCCTCCTGTTCAGCAGACCGCGT
GTCCCCCGGACAGCTATGAAACTCAAGTCAGACTAACTGCAGATGGTTGC
TGTACTTTGCCAACAAGATGCGAGTGTCTCTCTGGCTTATGTGGTTTCCC
CGTGTGTGAGGTGGGATCCACTCCCCGCATAGTCTCTCGTGGCGATGGGA
CACCTGGAAAGTGCTGTGATGTCTTTGAATGTGTTAATGATACAAAGCCA
GCCTGCGTATTTAACAATGTGGAATATTATGATGGAGACATGTTTCGAAT
GGACAACTGTCGGTTCTGTCGATGCCAAGGGGGCGTTGCCATCTGCTTCA
CCGCCCAGTGTGGTGAGATAAACTGCGAGAGGTACTACGTGCCCGAAGGA
GAGTGCTGCCCAGTGTGTGAAGATCCAGTGTATCCTTTTAATAATCCCGC
TGGCTGCTATGCCAATGGCCTGATCCTTGCCCACGGAGACCGGTGGCGGG
AAGACGACTGCACATTCTGCCAGTGCGTCAACGGTGAACGCCACTGCGTT
GCGACCGTCTGCGGACAGACCTGCACAAACCCTGTGAAAGTGCCTGGGGA
GTGTTGCCCTGTGTGCGAAGAACCAACCATCATCACAGTTGATCCACCTG
CATGTGGGGAGTTATCAAACTGCACTCTGACAGGGAAGGACTGCATTAAT
GGTTTCAAACGCGATCACAATGGTTGTCGGACCTGTCAGTGCATAAACAC
CGAGGAACTATGTTCAGAACGTAAACAAGGCTGCACCTTGAACTGTCCCT
TCGGTTTCCTTACTGATGCCCAAAACTGTGAGATCTGTGAGTGCCGCCCA
AGGCCCAAGAAGTGCAGACCCATAATCTGTGACAAGTATTGTCCACTTGG
ATTGCTGAAGAATAAGCACGGCTGTGACATCTGTCGCTGTAAGAAATGTC
CAGAGCTCTCATGCAGTAAGATCTGCCCCTTGGGTTTCCAGCAGGACAGT
CACGGCTGTCTTATCTGCAAGTGCAGAGAGGCCTCTGCTTCAGCTGGGCC
ACCCATCCTGTCGGGCACTTGTCTCACCGTGGATGGTCATCATCATAAAA
ATGAGGAGAGCTGGCACGATGGGTGCCGGGAATGCTACTGTCTCAATGGA
CGGGAAATGTGTGCCCTGATCACCTGCCCGGTGCCTGCCTGTGGCAACCC
CACCATTCACCCTGGACAGTGCTGCCCATCATGTGCAGATGACTTTGTGG
TGCAGAAGCCAGAGCTCAGTACTCCCTCCATTTGCCACGCCCCTGGAGGA
GAATACTTTGTGGAAGGAGAAACGTGGAACATTGACTCCTGTACTCAGTG
CACCTGCCACAGCGGACGGGTGCTGTGTGAGACAGAGGTGTGCCCACCGC
TGCTCTGCCAGAACCCCTCACGCACCCAGGATTCCTGCTGCCCACAGTGT
ACAGATCAACCTTTTCGGCCTTCCTTGTCCCGCAATAACAGCGTACCTAA
TTACTGCAAAAATGATGAAGGGGATATATTCCTGGCAGCTGAGTCCTGGA
AGCCTGACGTTTGTACCAGCTGCATCTGCATTGATAGCGTAATTAGCTGT
TTCTCTGAGTCCTGCCCTTCTGTATCCTGTGAAAGACCTGTCTTGAGAAA
AGGCCAGTGTTGTCCCTACTGCATAGAAGACACAATTCCAAAGAAGGTGG
TGTGCCACTTCAGTGGGAAGGCCTATGCCGACGAGGAGCGGTGGGACCTT
GACAGCTGCACCCACTGCTACTGCCTGCAGGGCCAGACCCTCTGCTCGAC
CGTCAGCTGCCCCCCTCTGCCCTGTGTTGAGCCCATCAACGTGGAAGGAA
GTTGCTGCCCAATGTGTCCAGAAATGTATGTCCCAGAACCAACCAATATA
CCCATTGAGAAGACAAACCATCGAGGAGAGGTTGACCTGGAGGTTCCCCT
GTGGCCCACGCCTAGTGAAAATGATATCGTCCATCTCCCTAGAGATATGG
GTCACCTCCAGGTAGATTACAGAGATAACAGGCTGCACCCAAGTGAAGAT
TCTTCACTGGACTCC

In certain embodiments, the disclosure relates to heteromultimers that comprise at least one CRIM1 polypeptide, which includes fragments, functional variants, and modified forms thereof. Preferably, CRIM1 polypeptides for use in accordance with the disclosure (e.g., heteromultimers comprising a CRIM1 polypeptide and uses thereof) are soluble (e.g., an extracellular domain of CRIM1). In other preferred embodiments, CRIM1 polypeptides for use in accordance with the disclosure bind to and/or inhibit (antagonize) activity (e.g., Smad signaling) of one or more TGF-beta superfamily ligands. In some embodiments, heteromultimers of the disclosure comprise at least one CRIM1 polypeptide that is at least 70%, 75%, 80%, 85%, 90%, 91%, 92%, 93%, 94%, 95%, 96%, 97%, 98%, 99%, or 100% identical to the amino acid sequence of SEQ ID NOs: 537 or 538. In some embodiments, heteromultimers of the disclosure comprise at least one CRIM1 polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 35-37 (e.g., amino acid residues 35, 36, or 37) of SEQ ID NO: 537, and ends at any one of amino acids 873-939 (e.g., amino acid residues 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, or 939) of SEQ ID NO: 537. In some embodiments, heteromultimers of the disclosure comprise at least one CRIM1 polypeptide that is at least 70%, 75%, 80%, 85%, 90%, 91%, 92%, 93%, 94%, 95%, 96%, 97%, 98%, 99%, or 100% identical to amino acids of 35-939 of SEQ ID NO: 537. In some embodiments, heteromultimers of the disclosure comprise at least one CRIM1 polypeptide that is at least 70%, 75%, 80%, 85%, 90%, 91%, 92%, 93%, 94%, 95%, 96%, 97%, 98%, 99%, or 100% identical to amino acids of 37-939 of SEQ ID NO: 537. In some embodiments, heteromultimers of the disclosure comprise at least one CRIM1 polypeptide that is at least 70%, 75%, 80%, 85%, 90%, 91%, 92%, 93%, 94%, 95%, 96%, 97%, 98%, 99%, or 100% identical to amino acids of 35-873 of SEQ ID NO: 537. In some embodiments, heteromultimers of the disclosure comprise at least one CRIM1 polypeptide that is at least 70%, 75%, 80%, 85%, 90%, 91%, 92%, 93%, 94%, 95%, 96%, 97%, 98%, 99%, or 100% identical to amino acids of 37-939 of SEQ ID NO: 537.

The term “CRIM2 polypeptide” includes polypeptides comprising any naturally occurring CRIM2 protein (encoded by KCP or one of its nonhuman orthologs) as well as any variants thereof (including mutants, fragments, fusions, and peptidomimetic forms) that retain a useful activity.

A human CRIM2 isoform 1 precursor protein sequence (NCBI Ref Seq NP_001129386.1) is as follows:

| (SEQ ID NO: 541) | |
|---|---|
| 1 | MAGVGAAALS LLLHLGALAL AAGAEGGAVP REPPGQQTTA HSSVLAGNSQ EQWHPLREWL |
| 61 | GRLEAAVMEL REQNKDLQTR VRQLESCECH PASPQCWGLG RAWPEGARWE PDACTACVCQ |
| 121 | DGAAHCGPQA HLPHCRGCSQ NGQTYGNGET FSPDACTTCR CLTGAVQCQG PSCSELNCLE |
| 181 | SCTPPGECCP ICCTEGGSHW EHGQEWTTPG DPCRICRCLE GHIQCRQREC ASLCPYPARP |
| 241 | LPGTCCPVCD GCFLNGREHR SGEPVGSGDP CSHCRCANGS VQCEPLPCPP VPCRHPGKIP |
| 301 | GQCCPVCDGC EYQGHQYQSQ ETFRLQERGL CVRCSCQAGE VSCEEQECPV TPCALPASGR |
| 361 | QLCPACELDG EEFAEGVQWE PDGRPCTACV CQDGVPKCGA VLCPPAPCQH PTQPPGACCP |
| 421 | SCDSCTYHSQ VYANGQNFTD ADSPCHACHC QDGTVTCSLV DCPPTTCARP QSGPGQCCPR |
| 481 | CPDCILEEEV FVDGESFSHP RDPCQECRCQ EGHAHCQPRP CPRAPCAHPL PGTCCPNDCS |
| 541 | GCAFGGKEYP SGADFPHPSD PCRLCRCLSG NVQCLARRCV PLPCPEPVLL PGECCPQCPA |
| 601 | PAGCPRPGAA HARHQEYFSP PGDPCRRCLC LDGSVSCQRL PCPPAPCAHP RQGPCCPSCD |
| 661 | GCLYQGKEFA SGERFPSPTA ACHLCLCWEG SVSCEPKACA PALCPFPARG DCCPDCDGCE |
| 721 | YLGESYLSNQ EFPDPREPCN LCTCLGGFVT CGRRPCEPPG CSHPLIPSGH CCPTCQGCRY |
| 781 | HGVTTASGET LPDPLDPTCS LCTCQEGSMR CQKKPCPPAL CPHPSPGPCF CPVCHSCLSQ |
| 841 | GREHQDGEEF EGPAGSCEWC RCQAGQVSCV RLQCPPLPCK LQVTERGSCC PRCRGCLAHG |
| 901 | EEHPEGSRWV PPDSACSSCV CHEGVVTCAR IQCISSCAQP RQGPHDCCPQ CSDCEHEGRK |
| 961 | YEPGESFQPG ADPCEVCICE PQPEGPPSLR CHRRQCPSLV GCPPSQLLPP GPQHCCPTCA |
| 1021 | EALSNCSEGL LGSELAPPDP CYTCQCQDLT WLCIHQACPE LSCPLSERHT PPGSCCPVCR |
| 1081 | APTQSCVHQG REVASGERWT VDTCTSCSCM AGTVRCQSQR CSPLSCGPDK APALSPGSCC |
| 1141 | PRCLPRPASC MAFGDPHYRT FDGRLLHFQG SCSYVLAKDC HSGDFSVHVT NDDRGRSGVA |
| 1201 | WTQEVAVLLG DMAVRLLQDG AVTVDGHPVA LPFLQEPLLY VELRGHTVIL HAQPGLQVLW |
| 1261 | DGQSQVEVSV PGSYQGRTCG LCGNFNGFAQ DDLQGPEGLL LPSEAAFGNS WQVSEGLWPG |
| 1321 | RPCSAGREVD PCRAAGYRAR REANARCGVL KSSPFSRCHA VVPPEPFFAA CVYDLCACGP |
| 1381 | GSSADACLCD ALEAYASHCR QAGVTPTWRG PTLCVVGCPL ERGFVFDECG PPCPRTCFNQ |
| 1441 | HIPLGELAAH CVRPCVPGCQ CPAGLVEHEA HCIPPEACPQ VLLTGDQPLG ARPSPSREPQ |
| 1501 | ETP |

The signal peptide is indicated by single underline.

A processed CRIM2 isoform 1 polypeptide sequence is as follows:

(SEQ ID NO: 542)

GAVPREPPGQQTTAHSSVLAGNSQEQWHPLREWLGRLEAAVMELREQNKD
LQTRVRQLESCECHPASPQCWGLGRAWPEGARWEPDACTACVCQDGAAHC
GPQAHLPHCRGCSQNGQTYGNGETFSPDACTTCRCLTGAVQCQGPSCSEL
NCLESCTPPGECCPICCTEGGSHWEHGQEWTTPGDPCRICRCLEGHIQCR
QRECASLCPYPARPLPGTCCPVCDGCFLNGREHRSGEPVGSGDPCSHCRC
ANGSVQCEPLPCPPVPCRHPGKIPGQCCPVCDGCEYQGHQYQSQETFRLQ
ERGLCVRCSCQAGEVSCEEQECPVTPCALPASGRQLCPACELDGEEFAEG
VQWEPDGRPCTACVCQDGVPKCGAVLCPPAPCQHPTQPPGACCPSCDSCT
YHSQVYANGQNFTDADSPCHACHCQDGTVTCSLVDCPPTTCARPQSGPGQ
CCPRCPDCILEEEVFVDGESFSHPRDPCQECRCQEGHAHCQPRPCPRAPC
AHPLPGTCCPNDCSGCAFGGKEYPSGADFPHPSDPCRLCRCLSGNVQCLA
RRCVPLPCPEPVLLPGECCPQCPAPAGCPRPGAAHARHQEYFSPPGDPCR
RCLCLDGSVSCQRLPCPPAPCAHPRQGPCCPSCDGCLYQGKEFASGERFP
SPTAACHLCLCWEGSVSCEPKACAPALCPFPARGDCCPDCDGCEYLGESY
LSNQEFPDPREPCNLCTCLGGFVTCGRRPCEPPGCSHPLIPSGHCCPTCQ
GCRYHGVTTASGETLPDPLDPTCSLCTCQEGSMRCQKKPCPPALCPHPSP
GPCFCPVCHSCLSQGREHQDGEEFEGPAGSCEWCRCQAGQVSCVRLQCPP
LPCKLQVTERGSCCPRCRGCLAHGEEHPEGSRWVPPDSACSSCVCHEGVV
TCARIQCISSCAQPRQGPHDCCPQCSDCEHEGRKYEPGESFQPGADPCEV
CICEPQPEGPPSLRCHRRQCPSLVGCPPSQLLPPGPQHCCPTCAEALSNC
SEGLLGSELAPPDPCYTCQCQDLTWLCIHQACPELSCPLSERHTPPGSCC
PVCRAPTQSCVHQGREVASGERWTVDTCTSCSCMAGTVRCQSQRCSPLSC
GPDKAPALSPGSCCPRCLPRPASCMAFGDPHYRTFDGRLLHFQGSCSYVL
AKDCHSGDFSVHVTNDDRGRSGVAWTQEVAVLLGDMAVRLLQDGAVTVDG
HPVALPFLQEPLLYVELRGHTVILHAQPGLQVLWDGQSQVEVSVPGSYQG
RTCGLCGNFNGFAQDDLQGPEGLLLPSEAAFGNSWQVSEGLWPGRPCSAG
REVDPCRAAGYRARREANARCGVLKSSPFSRCHAVVPPEPFFAACVYDLC
ACGPGSSADACLCDALEAYASHCRQAGVTPTWRGPTLCVVGCPLERGFVF
DECGPPCPRTCFNQHIPLGELAAHCVRPCVPGCQCPAGLVEHEAHCIPPE
ACPQVLLTGDQPLGARPSPSREPQETP

A nucleic acid sequence encoding unprocessed human CRIM2 isoform 1 precursor protein is shown below (SEQ ID NO: 543), corresponding to nucleotides 44-4552 of NCBI Reference Sequence NM_001135914.1. The signal sequence is underlined.

(SEQ ID NO: 543)

ATGGCCGGGGTCGGGGCCGCTGCGCTGTCCCTTCTCCTGCACCTCGGGGC
CCTGGCGCTGGCCGCGGGCGCGGAAGGTGGGGCTGTCCCCAGGGAGCCCC
CTGGGCAGCAGACAACTGCCCATTCCTCAGTCCTTGCTGGGAACTCCCAG
GAGCAGTGGCACCCCCTGCGAGAGTGGCTGGGGCGACTGGAGGCTGCAGT
GATGGAGCTCAGAGAACAGAATAAGGACCTGCAGACGAGGGTGAGGCAGC
TGGAGTCCTGTGAGTGCCACCCTGCATCTCCCCAGTGCTGGGGGCTGGGG
CGTGCCTGGCCCGAGGGGGCACGCTGGGAGCCTGACGCCTGCACAGCCTG
CGTCTGCCAGGATGGGGCCGCTCACTGTGGCCCCCAAGCACACCTGCCCC
ATTGCAGGGGCTGCAGCCAAAATGGCCAGACCTACGGCAACGGGGAGACC
TTCTCCCCAGATGCCTGCACCACCTGCCGCTGTCTGACAGGAGCCGTGCA
GTGCCAGGGGCCCTCGTGTTCAGAGCTCAACTGCTTGGAGAGCTGCACCC
CACCTGGGGAGTGCTGCCCCATCTGCTGCACAGAAGGTGGCTCTCACTGG
GAACATGGCCAAGAGTGGACAACACCTGGGGACCCCTGCCGAATCTGCCG
GTGCCTGGAGGGTCACATCCAGTGCCGCCAGCGAGAATGTGCCAGCCTGT
GTCCATACCCAGCCCGGCCCCTCCCAGGCACCTGCTGCCCTGTGTGTGAT
GGCTGTTTCCTAAACGGGCGGGAGCACCGCAGCGGGGAGCCTGTGGGCTC
AGGGGACCCCTGCTCGCACTGCCGCTGTGCTAATGGGAGTGTCCAGTGTG
AGCCTCTGCCCTGCCCGCCAGTGCCCTGCAGACACCCAGGCAAGATCCCT
GGGCAGTGCTGCCCTGTCTGCGATGGCTGTGAGTACCAGGGACACCAGTA
TCAGAGCCAGGAGACCTTCAGACTCCAAGAGCGGGGCCTCTGTGTCCGCT
GCTCCTGCCAGGCTGGCGAGGTCTCCTGTGAGGAGCAGGAGTGCCCAGTC
ACCCCCTGTGCCCTGCCTGCCTCTGGCCGCCAGCTCTGCCCAGCCTGTGA
GCTGGATGGAGAGGAGTTTGCTGAGGGAGTCCAGTGGGAGCCTGATGGTC
GGCCCTGCACCGCCTGCGTCTGTCAAGATGGGGTACCCAAGTGCGGGGCT
GTGCTCTGCCCCCCAGCCCCCTGCCAGCACCCCACCCAGCCCCCTGGTGC
CTGCTGCCCCAGCTGTGACAGCTGCACCTACCACAGCCAAGTGTATGCCA
ATGGGCAGAACTTCACGGATGCAGACAGCCCTTGCCATGCCTGCCACTGT
CAGGATGGAACTGTGACATGCTCCTTGGTTGACTGCCCTCCCACGACCTG
TGCCAGGCCCCAGAGTGGACCAGGCCAGTGTTGCCCCAGGTGCCCAGACT
GCATCCTGGAGGAAGAGGTGTTTGTGGACGGCGAGAGCTTCTCCCACCCC
CGAGACCCCTGCCAGGAGTGCCGATGCCAGGAAGGCCATGCCCACTGCCA
GCCTCGCCCCTGCCCCAGGGCCCCCTGTGCCCACCCGCTGCCTGGGACCT
GCTGCCCGAACGACTGCAGCGGCTGTGCCTTTGGCGGGAAAGAGTACCCC
AGCGGAGCGGACTTCCCCCACCCCTCTGACCCCTGCCGTCTGTGTCGCTG
TCTGAGCGGCAACGTGCAGTGCCTGGCCCGCCGCTGCGTGCCGCTGCCCT
GTCCAGAGCCTGTCCTGCTGCCGGGAGAGTGCTGCCCGCAGTGCCCAGCC
CCCGCCGGCTGCCCACGGCCCGGCGCGGCCCACGCCCGCCACCAGGAGTA
CTTCTCCCCGCCCGGCGATCCCTGCCGCCGCTGCCTCTGCCTCGACGGCT
CCGTGTCCTGCCAGCGGCTGCCCTGCCCGCCCGCGCCCTGCGCGCACCCG
CGCCAGGGGCCTTGCTGCCCCTCCTGCGACGGCTGCCTGTACCAGGGGAA
GGAGTTTGCCAGCGGGGAGCGCTTCCCATCGCCCACTGCTGCCTGCCACC
TCTGCCTTTGCTGGGAGGGCAGCGTGAGCTGCGAGCCCAAGGCATGTGCC
CCTGCACTGTGCCCCTTCCCTGCCAGGGGCGACTGCTGCCCTGACTGTGA
TGGCTGTGAGTACCTGGGGGAGTCCTACCTGAGTAACCAGGAGTTCCCAG
ACCCCCGAGAACCCTGCAACCTGTGTACCTGTCTTGGAGGCTTCGTGACC
TGCGGCCGCCGGCCCTGTGAGCCTCCGGGCTGCAGCCACCCACTCATCCC
CTCTGGGCACTGCTGCCCGACCTGCCAGGGATGCCGCTACCATGGCGTCA
CTACTGCCTCCGGAGAGACCCTTCCTGACCCACTTGACCCTACCTGCTCC
CTCTGCACCTGCCAGGAAGGTTCCATGCGCTGCCAGAAGAAGCCATGTCC
CCCAGCTCTCTGCCCCCACCCCTCTCCAGGCCCCTGCTTCTGCCCTGTTT
GCCACAGCTGTCTCTCTCAGGGCCGGGAGCACCAGGATGGGGAGGAGTTT
GAGGGACCAGCAGGCAGCTGTGAGTGGTGTCGCTGTCAGGCTGGCCAGGT
CAGCTGTGTGCGGCTGCAGTGCCCACCCCTTCCCTGCAAGCTCCAGGTCA
CCGAGCGGGGGAGCTGCTGCCCTCGCTGCAGAGGCTGCCTGGCTCATGGG
GAAGAGCACCCCGAAGGCAGTAGATGGGTGCCCCCCGACAGTGCCTGCTC
CTCCTGTGTGTGTCACGAGGGCGTCGTCACCTGTGCACGCATCCAGTGCA
TCAGCTCTTGCGCCCAGCCCCGCCAAGGGCCCCATGACTGCTGTCCTCAA
TGCTCTGACTGTGAGCATGAGGGCCGGAAGTACGAGCCTGGGGAGAGCTT
CCAGCCTGGGGCAGACCCCTGTGAAGTGTGCATCTGCGAGCCACAGCCTG
AGGGGCCTCCCAGCCTTCGCTGTCACCGGCGGCAGTGTCCCAGCCTGGTG
GGCTGCCCCCCCAGCCAGCTCCTGCCCCCTGGGCCCCAGCACTGCTGTCC
CACCTGTGCCGAGGCCTTGAGTAACTGTTCAGAGGGCCTGCTGGGATCTG
AGCTAGCCCCACCAGACCCCTGCTACACGTGCCAGTGCCAGGACCTGACA
TGGCTCTGCATCCACCAGGCTTGTCCTGAGCTCAGCTGTCCCCTCTCAGA
GCGCCACACTCCCCCTGGGAGCTGCTGCCCCGTATGCCGGGCTCCCACCC
AGTCCTGCGTGCACCAGGGCCGTGAGGTGGCCTCTGGAGAGCGCTGGACT
GTGGACACCTGCACCAGCTGCTCCTGCATGGCGGGCACCGTGCGTTGCCA
GAGCCAGCGCTGCTCACCGCTCTCGTGTGGCCCCGACAAGGCCCCTGCCC
TGAGTCCTGGCAGCTGCTGCCCCCGCTGCCTGCCTCGGCCCGCTTCCTGC
ATGGCCTTCGGAGACCCCCATTACCGCACCTTCGACGGCCGCCTGCTGCA
CTTCCAGGGCAGTTGCAGCTATGTGCTGGCCAAGGACTGCCACAGCGGGG
ACTTCAGTGTGCACGTGACCAATGATGACCGGGGCCGGAGCGGTGTGGCC
TGGACCCAGGAGGTGGCGGTGCTGCTGGGAGACATGGCCGTGCGGCTGCT
GCAGGACGGGGCAGTCACGGTGGATGGGCACCCGGTGGCCTTGCCCTTCC
TGCAGGAGCCGCTGCTGTATGTGGAGCTGCGAGGACACACTGTGATCCTG
CACGCCCAGCCCGGGCTCCAGGTGCTGTGGGATGGGCAGTCCCAGGTGGA
GGTGAGCGTACCTGGCTCCTACCAGGGCCGGACTTGTGGGCTCTGTGGGA
ACTTCAATGGCTTTGCCCAGGACGATCTGCAGGGCCCTGAGGGGCTGCTC
CTGCCCTCGGAGGCTGCGTTTGGGAATAGCTGGCAGGTCTCAGAGGGGCT
GTGGCCTGGCCGGCCCTGTTCTGCAGGCCGAGAGGTGGATCCGTGCCGGG
CAGCAGGTTACCGTGCCAGGCGTGAGGCCAATGCCCGGTGTGGGGTGCTG
AAGTCCTCCCCATTCAGTCGCTGCCATGCTGTGGTGCCACCGGAGCCCTT
CTTTGCCGCCTGTGTGTATGACCTGTGTGCCTGTGGCCCTGGCTCCTCCG
CTGATGCCTGCCTCTGTGATGCCCTGGAAGCCTACGCCAGTCACTGTCGC
CAGGCAGGAGTGACACCTACCTGGCGAGGCCCCACGCTGTGTGTGGTAGG
CTGCCCCCTGGAGCGTGGCTTCGTGTTTGATGAGTGCGGCCCACCCTGTC
CCCGCACCTGCTTCAATCAGCATATCCCCCTGGGGGAGCTGGCAGCCCAC
TGCGTGAGGCCCTGCGTGCCCGGCTGCCAGTGCCCTGCAGGCCTGGTGGA
GCATGAGGCCCACTGCATCCCACCCGAGGCCTGCCCCCAAGTCCTGCTCA
CTGGAGACCAGCCACTTGGTGCTCGGCCCAGCCCCAGCCGGGAGCCCCAG
GAGACACCC

A nucleic acid sequence encoding a processed human CRIM2 isoform 1 is shown below (SEQ ID NO: 544):

(SEQ ID NO: 544)

GGGGCTGTCCCCAGGGAGCCCCCTGGGCAGCAGACAACTGCCCATTCCTC
AGTCCTTGCTGGGAACTCCCAGGAGCAGTGGCACCCCCTGCGAGAGTGGC
TGGGGCGACTGGAGGCTGCAGTGATGGAGCTCAGAGAACAGAATAAGGAC
CTGCAGACGAGGGTGAGGCAGCTGGAGTCCTGTGAGTGCCACCCTGCATC
TCCCCAGTGCTGGGGGCTGGGGCGTGCCTGGCCCGAGGGGGCACGCTGGG
AGCCTGACGCCTGCACAGCCTGCGTCTGCCAGGATGGGGCCGCTCACTGT
GGCCCCCAAGCACACCTGCCCCATTGCAGGGGCTGCAGCCAAAATGGCCA
GACCTACGGCAACGGGGAGACCTTCTCCCCAGATGCCTGCACCACCTGCC
GCTGTCTGACAGGAGCCGTGCAGTGCCAGGGGCCCTCGTGTTCAGAGCTC
AACTGCTTGGAGAGCTGCACCCCACCTGGGGAGTGCTGCCCCATCTGCTG
CACAGAAGGTGGCTCTCACTGGGAACATGGCCAAGAGTGGACAACACCTG
GGGACCCCTGCCGAATCTGCCGGTGCCTGGAGGGTCACATCCAGTGCCGC
CAGCGAGAATGTGCCAGCCTGTGTCCATACCCAGCCCGGCCCCTCCCAGG
CACCTGCTGCCCTGTGTGTGATGGCTGTTTCCTAAACGGGCGGGAGCACC
GCAGCGGGGAGCCTGTGGGCTCAGGGGACCCCTGCTCGCACTGCCGCTGT
GCTAATGGGAGTGTCCAGTGTGAGCCTCTGCCCTGCCCGCCAGTGCCCTG
CAGACACCCAGGCAAGATCCCTGGGCAGTGCTGCCCTGTCTGCGATGGCT
GTGAGTACCAGGGACACCAGTATCAGAGCCAGGAGACCTTCAGACTCCAA
GAGCGGGGCCTCTGTGTCCGCTGCTCCTGCCAGGCTGGCGAGGTCTCCTG
TGAGGAGCAGGAGTGCCCAGTCACCCCCTGTGCCCTGCCTGCCTCTGGCC
GCCAGCTCTGCCCAGCCTGTGAGCTGGATGGAGAGGAGTTTGCTGAGGGA
GTCCAGTGGGAGCCTGATGGTCGGCCCTGCACCGCCTGCGTCTGTCAAGA
TGGGGTACCCAAGTGCGGGGCTGTGCTCTGCCCCCCAGCCCCCTGCCAGC
ACCCCACCCAGCCCCCTGGTGCCTGCTGCCCCAGCTGTGACAGCTGCACC
TACCACAGCCAAGTGTATGCCAATGGGCAGAACTTCACGGATGCAGACAG
CCCTTGCCATGCCTGCCACTGTCAGGATGGAACTGTGACATGCTCCTTGG
TTGACTGCCCTCCCACGACCTGTGCCAGGCCCCAGAGTGGACCAGGCCAG
TGTTGCCCCAGGTGCCCAGACTGCATCCTGGAGGAAGAGGTGTTTGTGGA
CGGCGAGAGCTTCTCCCACCCCCGAGACCCCTGCCAGGAGTGCCGATGCC
AGGAAGGCCATGCCCACTGCCAGCCTCGCCCCTGCCCCAGGGCCCCCTGT
GCCCACCCGCTGCCTGGGACCTGCTGCCCGAACGACTGCAGCGGCTGTGC
CTTTGGCGGGAAAGAGTACCCCAGCGGAGCGGACTTCCCCCACCCCTCTG
ACCCCTGCCGTCTGTGTCGCTGTCTGAGCGGCAACGTGCAGTGCCTGGCC
CGCCGCTGCGTGCCGCTGCCCTGTCCAGAGCCTGTCCTGCTGCCGGGAGA
GTGCTGCCCGCAGTGCCCAGCCCCCGCCGGCTGCCCACGGCCCGGCGCGG
CCCACGCCCGCCACCAGGAGTACTTCTCCCCGCCCGGCGATCCCTGCCGC
CGCTGCCTCTGCCTCGACGGCTCCGTGTCCTGCCAGCGGCTGCCCTGCCC
GCCCGCGCCCTGCGCGCACCCGCGCCAGGGGCCTTGCTGCCCCTCCTGCG
ACGGCTGCCTGTACCAGGGGAAGGAGTTTGCCAGCGGGGAGCGCTTCCCA
TCGCCCACTGCTGCCTGCCACCTCTGCCTTTGCTGGGAGGGCAGCGTGAG
CTGCGAGCCCAAGGCATGTGCCCCTGCACTGTGCCCCTTCCCTGCCAGGG
GCGACTGCTGCCCTGACTGTGATGGCTGTGAGTACCTGGGGGAGTCCTAC
CTGAGTAACCAGGAGTTCCCAGACCCCCGAGAACCCTGCAACCTGTGTAC
CTGTCTTGGAGGCTTCGTGACCTGCGGCCGCCGGCCCTGTGAGCCTCCGG
GCTGCAGCCACCCACTCATCCCCTCTGGGCACTGCTGCCCGACCTGCCAG
GGATGCCGCTACCATGGCGTCACTACTGCCTCCGGAGAGACCCTTCCTGA
CCCACTTGACCCTACCTGCTCCCTCTGCACCTGCCAGGAAGGTTCCATGC
GCTGCCAGAAGAAGCCATGTCCCCCAGCTCTCTGCCCCCACCCCTCTCCA
GGCCCCTGCTTCTGCCCTGTTTGCCACAGCTGTCTCTCTCAGGGCCGGGA
GCACCAGGATGGGGAGGAGTTTGAGGGACCAGCAGGCAGCTGTGAGTGGT
GTCGCTGTCAGGCTGGCCAGGTCAGCTGTGTGCGGCTGCAGTGCCCACCC
CTTCCCTGCAAGCTCCAGGTCACCGAGCGGGGGAGCTGCTGCCCTCGCTG
CAGAGGCTGCCTGGCTCATGGGGAAGAGCACCCCGAAGGCAGTAGATGGG
TGCCCCCCGACAGTGCCTGCTCCTCCTGTGTGTGTCACGAGGGCGTCGTC
ACCTGTGCACGCATCCAGTGCATCAGCTCTTGCGCCCAGCCCCGCCAAGG
GCCCCATGACTGCTGTCCTCAATGCTCTGACTGTGAGCATGAGGGCCGGA
AGTACGAGCCTGGGGAGAGCTTCCAGCCTGGGGCAGACCCCTGTGAAGTG
TGCATCTGCGAGCCACAGCCTGAGGGGCCTCCCAGCCTTCGCTGTCACCG
GCGGCAGTGTCCCAGCCTGGTGGGCTGCCCCCCCAGCCAGCTCCTGCCCC
CTGGGCCCCAGCACTGCTGTCCCACCTGTGCCGAGGCCTTGAGTAACTGT
TCAGAGGGCCTGCTGGGATCTGAGCTAGCCCCACCAGACCCCTGCTACAC
GTGCCAGTGCCAGGACCTGACATGGCTCTGCATCCACCAGGCTTGTCCTG
AGCTCAGCTGTCCCCTCTCAGAGCGCCACACTCCCCCTGGGAGCTGCTGC
CCCGTATGCCGGGCTCCCACCCAGTCCTGCGTGCACCAGGGCCGTGAGGT
GGCCTCTGGAGAGCGCTGGACTGTGGACACCTGCACCAGCTGCTCCTGCA
TGGCGGGCACCGTGCGTTGCCAGAGCCAGCGCTGCTCACCGCTCTCGTGT
GGCCCCGACAAGGCCCCTGCCCTGAGTCCTGGCAGCTGCTGCCCCCGCTG
CCTGCCTCGGCCCGCTTCCTGCATGGCCTTCGGAGACCCCCATTACCGCA
CCTTCGACGGCCGCCTGCTGCACTTCCAGGGCAGTTGCAGCTATGTGCTG
GCCAAGGACTGCCACAGCGGGGACTTCAGTGTGCACGTGACCAATGATGA
CCGGGGCCGGAGCGGTGTGGCCTGGACCCAGGAGGTGGCGGTGCTGCTGG
GAGACATGGCCGTGCGGCTGCTGCAGGACGGGGCAGTCACGGTGGATGGG
CACCCGGTGGCCTTGCCCTTCCTGCAGGAGCCGCTGCTGTATGTGGAGCT
GCGAGGACACACTGTGATCCTGCACGCCCAGCCCGGGCTCCAGGTGCTGT
GGGATGGGCAGTCCCAGGTGGAGGTGAGCGTACCTGGCTCCTACCAGGGC
CGGACTTGTGGGCTCTGTGGGAACTTCAATGGCTTTGCCCAGGACGATCT
GCAGGGCCCTGAGGGGCTGCTCCTGCCCTCGGAGGCTGCGTTTGGGAATA
GCTGGCAGGTCTCAGAGGGGCTGTGGCCTGGCCGGCCCTGTTCTGCAGGC
CGAGAGGTGGATCCGTGCCGGGCAGCAGGTTACCGTGCCAGGCGTGAGGC
CAATGCCCGGTGTGGGGTGCTGAAGTCCTCCCCATTCAGTCGCTGCCATG
CTGTGGTGCCACCGGAGCCCTTCTTTGCCGCCTGTGTGTATGACCTGTGT
GCCTGTGGCCCTGGCTCCTCCGCTGATGCCTGCCTCTGTGATGCCCTGGA
AGCCTACGCCAGTCACTGTCGCCAGGCAGGAGTGACACCTACCTGGCGAG
GCCCCACGCTGTGTGTGGTAGGCTGCCCCCTGGAGCGTGGCTTCGTGTTT
GATGAGTGCGGCCCACCCTGTCCCCGCACCTGCTTCAATCAGCATATCCC
CCTGGGGGAGCTGGCAGCCCACTGCGTGAGGCCCTGCGTGCCCGGCTGCC
AGTGCCCTGCAGGCCTGGTGGAGCATGAGGCCCACTGCATCCCACCCGAG
GCCTGCCCCCAAGTCCTGCTCACTGGAGACCAGCCACTTGGTGCTCGGCC
CAGCCCCAGCCGGGAGCCCCAGGAGACACCC

A human CRIM2 isoform 2 precursor protein sequence (NCBI Ref Seq NP_955381.2) is as follows:

| (SEQ ID NO: 545) | |
|---|---|
| 1 | MAGVGAAALS LLLHLGALAL AAGAEGGAVP REPPGQQTTA HSSVLAGNSQ EQWHPLREWL |
| 61 | GRLEAAVMEL REQNKDLQTR VRQLESCECH PASPQCWGLG RAWPEGARWE PDACTACVCQ |
| 121 | DGAAHCGPQA HLPHCRGCSQ NGQTYGNGET FSPDACTTCR CLEGTITCNQ KPCPRGPCPE |
| 181 | PGACCPHCKP GCDYEGQLYE EGVTFLSSSN PCLQCTCLRS RVRCMALKCP PSPCPEPVLR |
| 241 | PGHCCPTCQG CTEGGSHWEH GQEWTTPGDP CRICRCLEGH IQCRQRECAS LCPYPARPLP |
| 301 | GTCCPVCDGC FLNGREHRSG EPVGSGDPCS HCRCANGSVQ CEPLPCPPVP CRHPGKIPGQ |
| 361 | CCPVCDGCEY QGHQYQSQET FRLQERGLCV RCSCQAGEVS CEEQECPVTP CALPASGRQL |
| 421 | CPACELDGEE FAEGVQWEPD GRPCTACVCQ DGVPKCGAVL CPPAPCQHPT QPPGACCPSC |
| 481 | DSCTYHSQVY ANGQNFTDAD SPCHACHCQD GTVTCSLVDC PPTTCARPQS GPGQCCPRCP |
| 541 | DCILEEEVFV DGESFSHPRD PCQECRCQEG HAHCQPRPCP RAPCAHPLPG TCCPNDCSGC |
| 601 | AFGGKEYPSG ADFPHPSDPC RLCRCLSGNV QCLARRCVPL PCPEPVLLPG ECCPQCPAAP |
| 661 | APAGCPRPGA AHARHQEYFS PPGDPCRRCL CLDGSVSCQR LPCPPAPCAH PRQGPCCPSC |
| 721 | DGCLYQGKEF ASGERFPSPT AACHLCLCWE GSVSCEPKAC APALCPFPAR GDCCPDCDGE |
| 781 | GHGIGSCRGG MRETRGLGQN NLYCPRVDLK YLLQ |

A processed CRIM2 isoform 2 sequence is as follows:

(SEQ ID NO: 546)

AEGGAVPREPPGQQTTAHSSVLAGNSQEQWHPLREWLGRLEAAVMELREQ
NKDLQTRVRQLESCECHPASPQCWGLGRAWPEGARWEPDACTACVCQDGA
AHCGPQAHLPHCRGCSQNGQTYGNGETFSPDACTTCRCLEGTITCNQKPC
PRGPCPEPGACCPHCKPGCDYEGQLYEEGVTFLSSSNPCLQCTCLRSRVR
CMALKCPPSPCPEPVLRPGHCCPTCQGCTEGGSHWEHGQEWTTPGDPCRI
CRCLEGHIQCRQRECASLCPYPARPLPGTCCPVCDGCFLNGREHRSGEPV
GSGDPCSHCRCANGSVQCEPLPCPPVPCRHPGKIPGQCCPVCDGCEYQGH
QYQSQETFRLQERGLCVRCSCQAGEVSCEEQECPVTPCALPASGRQLCPA
CELDGEEFAEGVQWEPDGRPCTACVCQDGVPKCGAVLCPPAPCQHPTQPP
GACCPSCDSCTYHSQVYANGQNFTDADSPCHACHCQDGTVTCSLVDCPPT
TCARPQSGPGQCCPRCPDCILEEEVFVDGESFSHPRDPCQECRCQEGHAH
CQPRPCPRAPCAHPLPGTCCPNDCSGCAFGGKEYPSGADFPHPSDPCRLC
RCLSGNVQCLARRCVPLPCPEPVLLPGECCPQCPAAPAPAGCPRPGAAHA
RHQEYFSPPGDPCRRCLCLDGSVSCQRLPCPPAPCAHPRQGPCCPSCDGC
LYQGKEFASGERFPSPTAACHLCLCWEGSVSCEPKACAPALCPFPARGDC
CPDCDGEGHGIGSCRGGMRETRGLGQNNLYCPRVDLKYLLQ

A nucleic acid sequence encoding an unprocessed human CRIM2 isoform 2 precursor protein is shown below (SEQ ID NO: 547), corresponding to nucleotides 44-2485 of NCBI Reference Sequence NM_199349.2. The signal sequence is underlined.

(SEQ ID NO: 547)

ATGGCCGGGGTCGGGGCCGCTGCGCTGTCCCTTCTCCTGCACCTCGGGGC
CCTGGCGCTGGCCGCGGGCGCGGAAGGTGGGGCTGTCCCCAGGGAGCCCC
CTGGGCAGCAGACAACTGCCCATTCCTCAGTCCTTGCTGGGAACTCCCAG
GAGCAGTGGCACCCCCTGCGAGAGTGGCTGGGGCGACTGGAGGCTGCAGT
GATGGAGCTCAGAGAACAGAATAAGGACCTGCAGACGAGGGTGAGGCAGC
TGGAGTCCTGTGAGTGCCACCCTGCATCTCCCCAGTGCTGGGGGCTGGGG
CGTGCCTGGCCCGAGGGGGCACGCTGGGAGCCTGACGCCTGCACAGCCTG
CGTCTGCCAGGATGGGGCCGCTCACTGTGGCCCCCAAGCACACCTGCCCC
ATTGCAGGGGCTGCAGCCAAAATGGCCAGACCTACGGCAACGGGGAGACC
TTCTCCCCAGATGCCTGCACCACCTGCCGCTGTCTGGAAGGTACCATCAC
TTGCAACCAGAAGCCATGCCCAAGAGGACCCTGCCCTGAGCCAGGAGCAT
GCTGCCCGCACTGTAAGCCAGGCTGTGATTATGAGGGGCAGCTTTATGAG
GAGGGGGTCACCTTCCTGTCCAGCTCCAACCCTTGTCTACAGTGCACCTG
CCTGAGGAGCCGAGTTCGCTGCATGGCCCTGAAGTGCCCGCCTAGCCCCT
GCCCAGAGCCAGTGCTGAGGCCTGGGCACTGCTGCCCAACCTGCCAAGGC
TGCACAGAAGGTGGCTCTCACTGGGAACATGGCCAAGAGTGGACAACACC
TGGGGACCCCTGCCGAATCTGCCGGTGCCTGGAGGGTCACATCCAGTGCC
GCCAGCGAGAATGTGCCAGCCTGTGTCCATACCCAGCCCGGCCCCTCCCA
GGCACCTGCTGCCCTGTGTGTGATGGCTGTTTCCTAAACGGGCGGGAGCA
CCGCAGCGGGGAGCCTGTGGGCTCAGGGGACCCCTGCTCGCACTGCCGCT
GTGCTAATGGGAGTGTCCAGTGTGAGCCTCTGCCCTGCCCGCCAGTGCCC
TGCAGACACCCAGGCAAGATCCCTGGGCAGTGCTGCCCTGTCTGCGATGG
CTGTGAGTACCAGGGACACCAGTATCAGAGCCAGGAGACCTTCAGACTCC
AAGAGCGGGGCCTCTGTGTCCGCTGCTCCTGCCAGGCTGGCGAGGTCTCC
TGTGAGGAGCAGGAGTGCCCAGTCACCCCCTGTGCCCTGCCTGCCTCTGG
CCGCCAGCTCTGCCCAGCCTGTGAGCTGGATGGAGAGGAGTTTGCTGAGG
GAGTCCAGTGGGAGCCTGATGGTCGGCCCTGCACCGCCTGCGTCTGTCAA
GATGGGGTACCCAAGTGCGGGGCTGTGCTCTGCCCCCCAGCCCCCTGCCA
GCACCCCACCCAGCCCCCTGGTGCCTGCTGCCCCAGCTGTGACAGCTGCA
CCTACCACAGCCAAGTGTATGCCAATGGGCAGAACTTCACGGATGCAGAC
AGCCCTTGCCATGCCTGCCACTGTCAGGATGGAACTGTGACATGCTCCTT
GGTTGACTGCCCTCCCACGACCTGTGCCAGGCCCCAGAGTGGACCAGGCC
AGTGTTGCCCCAGGTGCCCAGACTGCATCCTGGAGGAAGAGGTGTTTGTG
GACGGCGAGAGCTTCTCCCACCCCCGAGACCCCTGCCAGGAGTGCCGATG
CCAGGAAGGCCATGCCCACTGCCAGCCTCGCCCCTGCCCCAGGGCCCCCT
GTGCCCACCCGCTGCCTGGGACCTGCTGCCCGAACGACTGCAGCGGCTGT
GCCTTTGGCGGGAAAGAGTACCCCAGCGGAGCGGACTTCCCCCACCCCTC
TGACCCCTGCCGTCTGTGTCGCTGTCTGAGCGGCAACGTGCAGTGCCTGG
CCCGCCGCTGCGTGCCGCTGCCCTGTCCAGAGCCTGTCCTGCTGCCGGGA
GAGTGCTGCCCGCAGTGCCCAGCCGCCCCAGCCCCCGCCGGCTGCCCACG
GCCCGGCGCGGCCCACGCCCGCCACCAGGAGTACTTCTCCCCGCCCGGCG
ATCCCTGCCGCCGCTGCCTCTGCCTCGACGGCTCCGTGTCCTGCCAGCGG
CTGCCCTGCCCGCCCGCGCCCTGCGCGCACCCGCGCCAGGGGCCTTGCTG
CCCCTCCTGCGACGGCTGCCTGTACCAGGGGAAGGAGTTTGCCAGCGGGG
AGCGCTTCCCATCGCCCACTGCTGCCTGCCACCTCTGCCTTTGCTGGGAG
GGCAGCGTGAGCTGCGAGCCCAAGGCATGTGCCCCTGCACTGTGCCCCTT
CCCTGCCAGGGGCGACTGCTGCCCTGACTGTGATGGTGAGGGTCATGGGA
TAGGGAGCTGCCGGGGTGGGATGCGGGAGACCAGAGGGCTGGGTCAGAAT
AATCTTTACTGCCCTAGGGTGGATCTAAAATATTTATTACAG

A nucleic acid sequence encoding a processed CRIM2 isoform 2 is shown below (SEQ ID NO: 548):

(SEQ ID NO: 548)

GCGGAAGGTGGGGCTGTCCCCAGGGAGCCCCCTGGGCAGCAGACAACTGC
CCATTCCTCAGTCCTTGCTGGGAACTCCCAGGAGCAGTGGCACCCCCTGC
GAGAGTGGCTGGGGCGACTGGAGGCTGCAGTGATGGAGCTCAGAGAACAG
AATAAGGACCTGCAGACGAGGGTGAGGCAGCTGGAGTCCTGTGAGTGCCA
CCCTGCATCTCCCCAGTGCTGGGGGCTGGGGCGTGCCTGGCCCGAGGGGG
CACGCTGGGAGCCTGACGCCTGCACAGCCTGCGTCTGCCAGGATGGGGCC
GCTCACTGTGGCCCCCAAGCACACCTGCCCCATTGCAGGGGCTGCAGCCA
AAATGGCCAGACCTACGGCAACGGGGAGACCTTCTCCCCAGATGCCTGCA
CCACCTGCCGCTGTCTGGAAGGTACCATCACTTGCAACCAGAAGCCATGC
CCAAGAGGACCCTGCCCTGAGCCAGGAGCATGCTGCCCGCACTGTAAGCC
AGGCTGTGATTATGAGGGGCAGCTTTATGAGGAGGGGGTCACCTTCCTGT
CCAGCTCCAACCCTTGTCTACAGTGCACCTGCCTGAGGAGCCGAGTTCGC
TGCATGGCCCTGAAGTGCCCGCCTAGCCCCTGCCCAGAGCCAGTGCTGAG
GCCTGGGCACTGCTGCCCAACCTGCCAAGGCTGCACAGAAGGTGGCTCTC
ACTGGGAACATGGCCAAGAGTGGACAACACCTGGGGACCCCTGCCGAATC
TGCCGGTGCCTGGAGGGTCACATCCAGTGCCGCCAGCGAGAATGTGCCAG
CCTGTGTCCATACCCAGCCCGGCCCCTCCCAGGCACCTGCTGCCCTGTGT
GTGATGGCTGTTTCCTAAACGGGCGGGAGCACCGCAGCGGGGAGCCTGTG
GGCTCAGGGGACCCCTGCTCGCACTGCCGCTGTGCTAATGGGAGTGTCCA
GTGTGAGCCTCTGCCCTGCCCGCCAGTGCCCTGCAGACACCCAGGCAAGA
TCCCTGGGCAGTGCTGCCCTGTCTGCGATGGCTGTGAGTACCAGGGACAC
CAGTATCAGAGCCAGGAGACCTTCAGACTCCAAGAGCGGGGCCTCTGTGT
CCGCTGCTCCTGCCAGGCTGGCGAGGTCTCCTGTGAGGAGCAGGAGTGCC
CAGTCACCCCCTGTGCCCTGCCTGCCTCTGGCCGCCAGCTCTGCCCAGCC
TGTGAGCTGGATGGAGAGGAGTTTGCTGAGGGAGTCCAGTGGGAGCCTGA
TGGTCGGCCCTGCACCGCCTGCGTCTGTCAAGATGGGGTACCCAAGTGCG
GGGCTGTGCTCTGCCCCCCAGCCCCCTGCCAGCACCCCACCCAGCCCCCT
GGTGCCTGCTGCCCCAGCTGTGACAGCTGCACCTACCACAGCCAAGTGTA
TGCCAATGGGCAGAACTTCACGGATGCAGACAGCCCTTGCCATGCCTGCC
ACTGTCAGGATGGAACTGTGACATGCTCCTTGGTTGACTGCCCTCCCACG
ACCTGTGCCAGGCCCCAGAGTGGACCAGGCCAGTGTTGCCCCAGGTGCCC
AGACTGCATCCTGGAGGAAGAGGTGTTTGTGGACGGCGAGAGCTTCTCCC
ACCCCCGAGACCCCTGCCAGGAGTGCCGATGCCAGGAAGGCCATGCCCAC
TGCCAGCCTCGCCCCTGCCCCAGGGCCCCCTGTGCCCACCCGCTGCCTGG
GACCTGCTGCCCGAACGACTGCAGCGGCTGTGCCTTTGGCGGGAAAGAGT
ACCCCAGCGGAGCGGACTTCCCCCACCCCTCTGACCCCTGCCGTCTGTGT
CGCTGTCTGAGCGGCAACGTGCAGTGCCTGGCCCGCCGCTGCGTGCCGCT
GCCCTGTCCAGAGCCTGTCCTGCTGCCGGGAGAGTGCTGCCCGCAGTGCC
CAGCCGCCCCAGCCCCCGCCGGCTGCCCACGGCCCGGCGCGGCCCACGCC
CGCCACCAGGAGTACTTCTCCCCGCCCGGCGATCCCTGCCGCCGCTGCCT
CTGCCTCGACGGCTCCGTGTCCTGCCAGCGGCTGCCCTGCCCGCCCGCGC
CCTGCGCGCACCCGCGCCAGGGGCCTTGCTGCCCCTCCTGCGACGGCTGC
CTGTACCAGGGGAAGGAGTTTGCCAGCGGGGAGCGCTTCCCATCGCCCAC
TGCTGCCTGCCACCTCTGCCTTTGCTGGGAGGGCAGCGTGAGCTGCGAGC
CCAAGGCATGTGCCCCTGCACTGTGCCCCTTCCCTGCCAGGGGCGACTGC
TGCCCTGACTGTGATGGTGAGGGTCATGGGATAGGGAGCTGCCGGGGTGG
GATGCGGGAGACCAGAGGGCTGGGTCAGAATAATCTTTACTGCCCTAGGG
TGGATCTAAAATATTTATTACAG

In certain embodiments, the disclosure relates to heteromultimers that comprise at least one CRIM2 polypeptide, which includes fragments, functional variants, and modified forms thereof. Preferably, CRIM2 polypeptides for use in accordance with the disclosure (e.g., heteromultimers comprising a CRIM2 polypeptide and uses thereof) are soluble (e.g., an extracellular domain of CRIM2). In other preferred embodiments, CRIM2 polypeptides for use in accordance with the disclosure bind to and/or inhibit (antagonize) activity (e.g., Smad signaling) of one or more TGF-beta superfamily ligands. In some embodiments, heteromultimers of the disclosure at least one CRIM2 polypeptide that is at least 70%, 75%, 80%, 85%, 90%, 91%, 92%, 93%, 94%, 95%, 96%, 97%, 98%, 99%, or 100% identical to the amino acid sequence of SEQ ID NOs: 541, 542, 545, or 546. In some embodiments, heteromultimers of the disclosure comprise at least one CRIM2 polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 26-138 (e.g., amino acid residues 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, and 138) of SEQ ID NO: 541, and ends at any one of amino acids 1298-1503 (e.g., amino acid residues 1298, 1299, 1300, 1301, 1302, 1303, 1304, 1305, 1306, 1307, 1308, 1309, 1310, 1311, 1312, 1313, 1314, 1315, 1316, 1317, 1318, 1319, 1320, 1321, 1322, 1323, 1324, 1325, 1326, 1327, 1328, 1329, 1330, 1331, 1332, 1333, 1334, 1335, 1335, 1336, 1337, 1338, 1339, 1340, 1341, 1342, 1343, 1344, 1345, 1346, 1347, 1348, 1349, 1350, 1351, 1352, 1353, 1354, 1355, 1356, 1357, 1358, 1359, 1360, 1361, 1362, 1363, 1364, 1365, 1366, 1367, 1368, 1369, 1370, 1371, 1372, 1373, 1374, 1375, 1376, 1377, 1378, 1379, 1380, 1381, 1382, 1383, 1384, 1385, 1386, 1387, 1388, 1389, 1390, 1391, 1392, 1393, 1394, 1395, 1396, 1397, 1398, 1399, 1400, 1401, 1402, 1403, 1404, 1405, 1406, 1407, 1408, 1409, 1410, 1411, 1412, 1413, 1414, 1415, 1416, 1417, 1418, 1419, 1420, 1421, 1422, 1423, 1424, 1425, 1426, 1427, 1428, 1429, 1430, 1431, 1432, 1433, 1434, 1435, 1435, 1436, 1437, 1438, 1439, 1440, 1441, 1442, 1443, 1444, 1445, 1446, 1447, 1448, 1349, 1450, 1451, 1452, 1453, 1454, 1455, 1456, 1457, 1458, 1459, 1460, 1461, 1462, 1463, 1464, 1465, 1466, 1467, 1468, 1469, 1470, 1471, 1472, 1473, 1474, 1475, 1476, 1477, 1478, 1479, 1480, 1481, 1482, 1483, 1484, 1485, 1486, 1487, 1488, 1489, 1490, 1491, 1492, 1493, 1494, 1495, 1496, 1497, 1498, 1499, 1500, 1501, 1502, or 1503) of SEQ ID NO: 541. In some embodiments, heteromultimers of the disclosure comprise at least one CRIM2 polypeptide that is at least 70%, 75%, 80%, 85%, 90%, 91%, 92%, 93%, 94%, 95%, 96%, 97%, 98%, 99%, or 100% identical to amino acids of 26-1298 of SEQ ID NO: 541. In some embodiments, heteromultimers of the disclosure comprise at least one CRIM2 polypeptide that is at least 70%, 75%, 80%, 85%, 90%, 91%, 92%, 93%, 94%, 95%, 96%, 97%, 98%, 99%, or 100% identical to amino acids of 26-1503 of SEQ ID NO: 541. In some embodiments, heteromultimers of the disclosure comprise at least one CRIM2 polypeptide that is at least 70%, 75%, 80%, 85%, 90%, 91%, 92%, 93%, 94%, 95%, 96%, 97%, 98%, 99%, or 100% identical to amino acids of 138-1298 of SEQ ID NO: 541. In some embodiments, heteromultimers of the disclosure comprise at least one CRIM2 polypeptide that is at least 70%, 75%, 80%, 85%, 90%, 91%, 92%, 93%, 94%, 95%, 96%, 97%, 98%, 99%, or 100% identical to amino acids of 138-1503 of SEQ ID NO: 541. In some embodiments, heteromultimers of the disclosure comprise at least one CRIM2 polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 24-138 (e.g., amino acid residues 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, or 138) of SEQ ID NO: 545, and ends at any one of amino acids 539-814 (e.g., amino acid residues 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 414, 615, 616, 617, 618, 619, 620, 621, 622, 623, 624, 625, 626, 627, 628, 629, 630, 631, 632, 633, 634, 635, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 405, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, or 814) of SEQ ID NO: 545. In some embodiments, heteromultimers of the disclosure comprise at least one CRIM2 polypeptide that is at least 70%, 75%, 80%, 85%, 90%, 91%, 92%, 93%, 94%, 95%, 96%, 97%, 98%, 99%, or 100% identical to amino acids of 24-539 of SEQ ID NO: 545. In some embodiments, heteromultimers of the disclosure comprise at least one CRIM2 polypeptide that is at least 70%, 75%, 80%, 85%, 90%, 91%, 92%, 93%, 94%, 95%, 96%, 97%, 98%, 99%, or 100% identical to amino acids of 24-814 of SEQ ID NO: 545. In some embodiments, heteromultimers of the disclosure comprise at least one CRIM2 polypeptide that is at least 70%, 75%, 80%, 85%, 90%, 91%, 92%, 93%, 94%, 95%, 96%, 97%, 98%, 99%, or 100% identical to amino acids of 138-539 of SEQ ID NO: 545. In some embodiments, heteromultimers of the disclosure comprise at least one CRIM2 polypeptide that is at least 70%, 75%, 80%, 85%, 90%, 91%, 92%, 93%, 94%, 95%, 96%, 97%, 98%, 99%, or 100% identical to amino acids of 138-814 of SEQ ID NO: 545. In some embodiments, heteromultimers of the disclosure comprise at least one CRIM2 polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 27-87 (e.g., amino acid residues 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, and 87) of SEQ ID NO: 541, and ends at any one of amino acids 1478-1503 (e.g., amino acid residues 1479, 1480, 1481, 1482, 1483, 1484, 1485, 1486, 1487, 1488, 1489, 1490, 1491, 1492, 1493, 1494, 1495, 1496, 1497, 1498, 1499, 1500, 1501, 1502, or 1503) of SEQ ID NO: 541. In some embodiments, heteromultimers of the disclosure comprise at least one CRIM2 polypeptide that is at least 70%, 75%, 80%, 85%, 90%, 91%, 92%, 93%, 94%, 95%, 96%, 97%, 98%, 99%, or 100% identical to amino acids of 27-1503 of SEQ ID NO: 541. In some embodiments, heteromultimers of the disclosure comprise at least one CRIM2 polypeptide that is at least 70%, 75%, 80%, 85%, 90%, 91%, 92%, 93%, 94%, 95%, 96%, 97%, 98%, 99%, or 100% identical to amino acids of 87-1478 of SEQ ID NO: 541. In some embodiments, heteromultimers of the disclosure comprise at least one CRIM2 polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 24-87 (e.g., amino acid residues 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, and 87) of SEQ ID NO: 545, and ends at any one of amino acids 804-814 (e.g., amino acid residues 804, 805, 806, 807, 808, 809, 810, 811, 812, 813, or 814) of SEQ ID NO: 545. In some embodiments, heteromultimers of the disclosure comprise at least one CRIM2 polypeptide that is at least 70%, 75%, 80%, 85%, 90%, 91%, 92%, 93%, 94%, 95%, 96%, 97%, 98%, 99%, or 100% identical to amino acids of 24-814 of SEQ ID NO: 545. In some embodiments, heteromultimers of the disclosure comprise at least one CRIM2 polypeptide that is at least 70%, 75%, 80%, 85%, 90%, 91%, 92%, 93%, 94%, 95%, 96%, 97%, 98%, 99%, or 100% identical to amino acids of 87-804 of SEQ ID NO: 545.

The term “BAMBI polypeptide” includes polypeptides comprising any naturally occurring BAMBI protein (encoded by BAMBI or one of its nonhuman orthologs) as well as any variants thereof (including mutants, fragments, fusions, and peptidomimetic forms) that retain a useful activity.

The human BAMBI precursor protein sequence (NCBI Ref Seq NP_036474.1) is as follows:

| | (SEQ ID NO: 549) |
|---|---|
| 1 | MDRHSSYIFI WLQLELCAMA VLLTKGEIRC YCDAAHCVAT GYMCKSELSA CFSRLLDPQN |
| 61 | SNSPLTHGCL DSLASTTDIC QAKQARNHSG TTIPTLECCH EDMCNYRGLH DVLSPPRGEA |
| 121 | 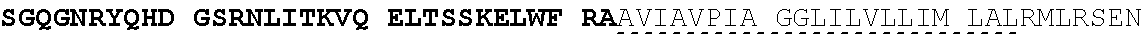 |
| 181 | KRLQDQRQQM LSRLHYSFHG HHSKKGQVAK LDLECMVPVS GHENCCLTCD KMRQADLSND |
| 241 | KILSLVHWGM YSGHGKLEFV |

The signal peptide is indicated by single underline, the extracellular domain is indicated in bold font, and the transmembrane domain is indicated by dotted underline.

A processed BAMBI polypeptide sequence is as follows:

(SEQ ID NO: 550)

VLLTKGEIRCYCDAAHCVATGYMCKSELSACFSRLLDPQNSNSPLTHGCL
DSLASTTDICQAKQARNHSGTTIPTLECCHEDMCNYRGLHDVLSPPRGEA
SGQGNRYQHDGSRNLITKVQELTSSKELWFRA

A nucleic acid sequence encoding unprocessed human BAMBI precursor protein is shown below (SEQ ID NO: 551), corresponding to nucleotides 404-1183 of NCBI Reference Sequence NM_012342.2. The signal sequence is indicated by solid underline and the transmembrane domain by dotted underline.

(SEQ ID NO: 551)
ATGGATCGCCACTCCAGCTACATCTTCATCTGGCTGCAGCTGGAGCTCTGCGCCATGGCCGTGCTGCTCACCAAA
GGTGAAATTCGATGCTACTGTGATGCTGCCCACTGTGTAGCCACTGGTTATATGTGTAAATCTGAGCTCAGCGCC
TGCTTCTCTAGACTTCTTGATCCTCAGAACTCAAATTCCCCACTCACCCATGGCTGCCTGGACTCTCTTGCAAGC
ACGACAGACATCTGCCAAGCCAAACAGGCCCGAAACCACTCTGGCACCACCATACCCACATTGGAATGCTGTCAT
GAAGACATGTGCAATTACAGAGGGCTGCACGATGTTCTCTCTCCTCCCAGGGGTGAGGCCTCAGGACAAGGAAAC
AGGTATCAGCATGATGGTAGCAGAAACCTTATCACCAAGGTGCAGGAGCTGACTTCTTCCAAAGAGTTGTGGTTC

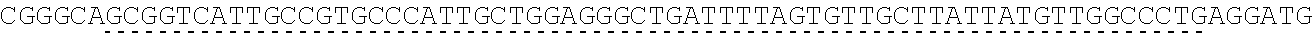

CTTCGAAGTGAAAATAAGAGGCTGCAGGATCAGCGGCAACAGATGCTCTCCCGTTTGCACTACAGCTTTCACGGA
CACCATTCCAAAAAGGGGCAGGTTGCAAAGTTAGACTTGGAATGCATGGTGCCGGTCAGTGGGCACGAGAACTGC
TGTCTGACCTGTGATAAAATGAGACAAGCAGACCTCAGCAACGATAAGATCCTCTCGCTTGTTCACTGGGGCATG
TACAGTGGGCACGGGAAGCTGGAATTCGTA

A nucleic acid sequence encoding a processed extracellular BAMBI is shown below (SEQ ID NO: 552):

(SEQ ID NO: 552)

GTGCTGCTCACCAAAGGTGAAATTCGATGCTACTGTGATGCTGCCCACTG
TGTAGCCACTGGTTATATGTGTAAATCTGAGCTCAGCGCCTGCTTCTCTA
GACTTCTTGATCCTCAGAACTCAAATTCCCCACTCACCCATGGCTGCCTG
GACTCTCTTGCAAGCACGACAGACATCTGCCAAGCCAAACAGGCCCGAAA
CCACTCTGGCACCACCATACCCACATTGGAATGCTGTCATGAAGACATGT
GCAATTACAGAGGGCTGCACGATGTTCTCTCTCCTCCCAGGGGTGAGGCC
TCAGGACAAGGAAACAGGTATCAGCATGATGGTAGCAGAAACCTTATCAC
CAAGGTGCAGGAGCTGACTTCTTCCAAAGAGTTGTGGTTCCGGGCA

In certain embodiments, the disclosure relates to heteromultimers that comprise at least one BAMBI polypeptide, which includes fragments, functional variants, and modified forms thereof. Preferably, BAMBI polypeptides for use in accordance with the disclosure (e.g., heteromultimers comprising a BAMBI polypeptide and uses thereof) are soluble (e.g., an extracellular domain of BAMBI). In other preferred embodiments, BAMBI polypeptides for use in accordance with disclosure bind to and/or inhibit (antagonize) activity (e.g., Smad signaling) of one or more TGF-beta superfamily ligands. In some embodiments, heteromultimers of the disclosure comprise at least one BAMBI polypeptide that is at least 70%, 75%, 80%, 85%, 90%, 91%, 92%, 93%, 94%, 95%, 96%, 97%, 98%, 99%, or 100% identical to the amino acid sequence of SEQ ID NOs: 549 or 550. In some embodiments, heteromultimers of the disclosure comprise at least one BAMBI polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 21-30 (e.g., amino acid residues 21, 22, 23, 24, 25, 26, 27, 28, 29, or 30) of SEQ ID NO: 549, and ends at any one of amino acids 104-152 (e.g., amino acid residues 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, or 152) of SEQ ID NO: 549. In some embodiments, heteromultimers of the disclosure comprise at least one BAMBI polypeptide that is at least 70%, 75%, 80%, 85%, 90%, 91%, 92%, 93%, 94%, 95%, 96%, 97%, 98%, 99%, or 100% identical to amino acids of 21-104 of SEQ ID NO: 549. In some embodiments, heteromultimers of the disclosure comprise at least one BAMBI polypeptide that is at least 70%, 75%, 80%, 85%, 90%, 91%, 92%, 93%, 94%, 95%, 96%, 97%, 98%, 99%, or 100% identical to amino acids of 21-152 of SEQ ID NO: 549. In some embodiments, heteromultimers of the disclosure comprise at least one BAMBI polypeptide that is at least 70%, 75%, 80%, 85%, 90%, 91%, 92%, 93%, 94%, 95%, 96%, 97%, 98%, 99%, or 100% identical to amino acids of 30-104 of SEQ ID NO: 549. In some embodiments, heteromultimers of the disclosure comprise at least one BAMBI polypeptide that is at least 70%, 75%, 80%, 85%, 90%, 91%, 92%, 93%, 94%, 95%, 96%, 97%, 98%, 99%, or 100% identical to amino acids of 30-152 of SEQ ID NO: 549. In some embodiments, heteromultimers of the disclosure comprise at least one BAMBI polypeptide that is at least 70%, 75%, 80%, 85%, 90%, 91%, 92%, 93%, 94%, 95%, 96%, 97%, 98%, 99%, or 100% identical to amino acids of 27-152 of SEQ ID NO: 549.

The term “BMPER polypeptide” includes polypeptides comprising any naturally occurring BMPER protein (encoded by BMPER or one of its nonhuman orthologs) as well as any variants thereof (including mutants, fragments, fusions, and peptidomimetic forms) that retain a useful activity.

A human BMPER precursor protein sequence (NCBI Ref Seq NP_597725.1) is as follows:

| (SEQ ID NO: 553) | |
|---|---|
| 1 | MLWFSGVGAL AERYCRRSPG ITCCVLLLLN CSGVPMSLAS SFLTGSVAKC ENEGEVLQIP |
| 61 | FITDNPCIMC VCLNKEVTCK REKCPVLSRD CALAIKQRGA CCEQCKGCTY EGNTYNSSFK |
| 121 | WQSPAEPCVL RQCQEGVVTE SGVRCVVHCK NPLEHLGMCC PTCPGCVFEG VQYQEGEEFQ |
| 181 | PEGSKCTKCS CTGGRTQCVR EVCPILSCPQ HLSHIPPGQC CPKCLGQRKV FDLPFGSCLF |
| 241 | RSDVYDNGSS FLYDNCTACT CRDSTVVCKR KCSHPGGCDQ GQEGCCEECL LRVPPEDIKV |
| 301 | CKFGNKIFQD GEMWSSINCT ICACVKGRTE CRNKQCIPIS SCPQGKILNR KGCCPICTEK |
| 361 | PGVCTVFGDP HYNTFDGRTF NFQGTCQYVL TKDCSSPASP FQVLVKNDAR RTRSFSWTKS |
| 421 | VELVLGESRV SLQQHLTVRW NGSRIALPCR APHFHIDLDG YLLKVTTKAG LEISWDGDSF |
| 481 | VEVMAAPHLK GKLCGLCGNY NGHKRDDLIG GDGNFKFDVD DFAESWRVES NEFCNRPQRK |
| 541 | PVPELCQGTV KVKLRAHREC QKLKSWEFQT CHSTVDYATF YRSCVTDMCE CPVHKNCYCE |
| 601 | SFLAYTRACQ REGIKVHWEP QQNCAATQCK HGAVYDTCGP GCIKTCDNWN EIGPCNKPCV |
| 661 | AGCHCPANLV LHKGRCIKPV LCPQR |

The signal peptide is indicated by a single underline.

A mature BMPER polypeptide sequence is as follows:

(SEQ ID NO: 554)

SSFLTGSVAKCENEGEVLQIPFITDNPCIMCVCLNKEVTCKREKCPVLSR
DCALAIKQRGACCEQCKGCTYEGNTYNSSFKWQSPAEPCVLRQCQEGVVT
ESGVRCVVHCKNPLEHLGMCCPTCPGCVFEGVQYQEGEEFQPEGSKCTKC
SCTGGRTQCVREVCPILSCPQHLSHIPPGQCCPKCLGQRKVFDLPFGSCL
FRSDVYDNGSSFLYDNCTACTCRDSTVVCKRKCSHPGGCDQGQEGCCEEC
LLRVPPEDIKVCKFGNKIFQDGEMWSSINCTICACVKGRTECRNKQCIPI
SSCPQGKILNRKGCCPICTEKPGVCTVFGDPHYNTFDGRTFNFQGTCQYV
LTKDCSSPASPFQVLVKNDARRTRSFSWTKSVELVLGESRVSLQQHLTVR
WNGSRIALPCRAPHFHIDLDGYLLKVTTKAGLEISWDGDSFVEVMAAPHL
KGKLCGLCGNYNGHKRDDLIGGDGNFKFDVDDFAESWRVESNEFCNRPQR
KPVPELCQGTVKVKLRAHRECQKLKSWEFQTCHSTVDYATFYRSCVTDMC
ECPVHKNCYCESFLAYTRACQREGIKVHWEPQQNCAATQCKHGAVYDTCG
PGCIKTCDNWNEIGPCNKPCVAGCHCPANLVLHKGRCIKPVLCPQR

A nucleic acid sequence encoding unprocessed human BMPER precursor protein is shown below (SEQ ID NO: 555), corresponding to nucleotides 375-2429 of NCBI Reference Sequence NM_133468.4. The signal sequence is underlined.

(SEQ ID NO: 555)

ATGCTCTGGTTCTCCGGCGTCGGGGCTCTGGCTGAGCGTTACTGCCGCCG
CTCGCCTGGGATTACGTGCTGCGTCTTGCTGCTACTCAATTGCTCGGGGG
TCCCCATGTCTCTGGCTTCCTCCTTCTTGACAGGTTCTGTTGCAAAATGT
GAAAATGAAGGTGAAGTCCTCCAGATTCCATTTATCACAGACAACCCTTG
CATAATGTGTGTCTGCTTGAACAAGGAAGTGACATGTAAGAGAGAGAAGT
GCCCCGTGCTGTCCCGAGACTGTGCCCTGGCCATCAAGCAGAGGGGAGCC
TGTTGTGAACAGTGCAAAGGTTGCACCTATGAAGGAAATACCTATAACAG
CTCCTTCAAATGGCAGAGCCCGGCTGAGCCTTGTGTTCTACGCCAGTGCC
AGGAGGGCGTTGTCACAGAGTCTGGGGTGCGCTGTGTTGTTCATTGTAAA
AACCCTTTGGAGCATCTGGGAATGTGCTGCCCCACATGTCCAGGCTGTGT
GTTTGAGGGTGTGCAGTATCAAGAAGGGGAGGAATTTCAGCCAGAAGGAA
GCAAATGTACCAAGTGTTCCTGCACTGGAGGCAGGACACAATGTGTGAGA
GAAGTCTGTCCCATTCTCTCCTGTCCCCAGCACCTTAGTCACATACCCCC
AGGACAGTGCTGCCCCAAATGTTTGGGTCAGAGGAAAGTGTTTGACCTCC
CTTTTGGGAGCTGCCTCTTTCGAAGTGATGTTTATGACAATGGATCCTCA
TTTCTGTACGATAACTGCACAGCTTGTACCTGCAGGGACTCTACTGTGGT
TTGCAAGAGGAAGTGCTCCCACCCTGGTGGCTGTGACCAAGGCCAGGAGG
GCTGTTGTGAAGAGTGCCTCCTACGAGTGCCCCCAGAAGACATCAAAGTA
TGCAAATTTGGCAACAAGATTTTCCAGGATGGAGAGATGTGGTCCTCTAT
CAATTGTACCATCTGTGCTTGTGTGAAAGGCAGGACGGAGTGTCGCAATA
AGCAGTGCATTCCCATCAGTAGCTGCCCACAGGGCAAAATTCTCAACAGA
AAAGGATGCTGTCCTATTTGCACTGAAAAGCCCGGCGTTTGCACGGTGTT
TGGAGATCCCCACTACAACACTTTTGACGGTCGGACATTTAACTTTCAGG
GGACGTGTCAGTACGTTTTGACAAAAGACTGCTCCTCCCCTGCCTCGCCC
TTCCAGGTGCTGGTGAAGAACGACGCCCGCCGGACACGCTCCTTCTCGTG
GACCAAGTCGGTGGAGCTGGTGCTGGGCGAGAGCAGGGTCAGCCTGCAGC
AGCACCTCACCGTGCGCTGGAACGGCTCGCGCATCGCGCTCCCCTGCCGC
GCGCCACACTTCCACATCGACCTGGATGGCTACCTCTTGAAAGTGACCAC
CAAAGCAGGTTTGGAAATATCTTGGGATGGAGACAGTTTTGTAGAAGTCA
TGGCTGCGCCGCATCTCAAGGGCAAGCTCTGTGGTCTTTGTGGCAACTAC
AATGGACATAAACGTGATGACTTAATTGGTGGAGATGGAAACTTCAAGTT
TGATGTGGATGACTTTGCTGAATCTTGGAGGGTGGAGTCCAATGAGTTCT
GCAACAGACCTCAGAGAAAGCCAGTGCCTGAACTGTGTCAAGGGACAGTC
AAGGTAAAGCTCCGGGCCCATCGAGAATGCCAAAAGCTCAAATCCTGGGA
GTTTCAGACCTGCCACTCGACTGTGGACTACGCCACTTTCTACCGGTCCT
GTGTGACAGACATGTGTGAATGTCCAGTCCATAAAAACTGTTATTGCGAG
TCATTTTTGGCATATACCCGGGCCTGCCAGAGAGAGGGCATCAAAGTCCA
CTGGGAGCCTCAGCAGAATTGTGCAGCCACCCAGTGTAAGCATGGTGCTG
TGTACGATACCTGTGGTCCGGGATGTATCAAGACGTGTGACAACTGGAAT
GAAATTGGTCCATGCAACAAGCCGTGCGTTGCTGGGTGCCACTGTCCAGC
AAACTTGGTCCTTCACAAGGGAAGGTGCATCAAGCCAGTCCTTTGTCCCC
AGCGG

A nucleic acid sequence encoding a processed BMPER is shown below (SEQ ID NO: 556):

(SEQ ID NO: 556)

TCCTCCTTCTTGACAGGTTCTGTTGCAAAATGTGAAAATGAAGGTGAAGT
CCTCCAGATTCCATTTATCACAGACAACCCTTGCATAATGTGTGTCTGCT
TGAACAAGGAAGTGACATGTAAGAGAGAGAAGTGCCCCGTGCTGTCCCGA
GACTGTGCCCTGGCCATCAAGCAGAGGGGAGCCTGTTGTGAACAGTGCAA
AGGTTGCACCTATGAAGGAAATACCTATAACAGCTCCTTCAAATGGCAGA
GCCCGGCTGAGCCTTGTGTTCTACGCCAGTGCCAGGAGGGCGTTGTCACA
GAGTCTGGGGTGCGCTGTGTTGTTCATTGTAAAAACCCTTTGGAGCATCT
GGGAATGTGCTGCCCCACATGTCCAGGCTGTGTGTTTGAGGGTGTGCAGT
ATCAAGAAGGGGAGGAATTTCAGCCAGAAGGAAGCAAATGTACCAAGTGT
TCCTGCACTGGAGGCAGGACACAATGTGTGAGAGAAGTCTGTCCCATTCT
CTCCTGTCCCCAGCACCTTAGTCACATACCCCCAGGACAGTGCTGCCCCA
AATGTTTGGGTCAGAGGAAAGTGTTTGACCTCCCTTTTGGGAGCTGCCTC
TTTCGAAGTGATGTTTATGACAATGGATCCTCATTTCTGTACGATAACTG
CACAGCTTGTACCTGCAGGGACTCTACTGTGGTTTGCAAGAGGAAGTGCT
CCCACCCTGGTGGCTGTGACCAAGGCCAGGAGGGCTGTTGTGAAGAGTGC
CTCCTACGAGTGCCCCCAGAAGACATCAAAGTATGCAAATTTGGCAACAA
GATTTTCCAGGATGGAGAGATGTGGTCCTCTATCAATTGTACCATCTGTG
CTTGTGTGAAAGGCAGGACGGAGTGTCGCAATAAGCAGTGCATTCCCATC
AGTAGCTGCCCACAGGGCAAAATTCTCAACAGAAAAGGATGCTGTCCTAT
TTGCACTGAAAAGCCCGGCGTTTGCACGGTGTTTGGAGATCCCCACTACA
ACACTTTTGACGGTCGGACATTTAACTTTCAGGGGACGTGTCAGTACGTT
TTGACAAAAGACTGCTCCTCCCCTGCCTCGCCCTTCCAGGTGCTGGTGAA
GAACGACGCCCGCCGGACACGCTCCTTCTCGTGGACCAAGTCGGTGGAGC
TGGTGCTGGGCGAGAGCAGGGTCAGCCTGCAGCAGCACCTCACCGTGCGC
TGGAACGGCTCGCGCATCGCGCTCCCCTGCCGCGCGCCACACTTCCACAT
CGACCTGGATGGCTACCTCTTGAAAGTGACCACCAAAGCAGGTTTGGAAA
TATCTTGGGATGGAGACAGTTTTGTAGAAGTCATGGCTGCGCCGCATCTC
AAGGGCAAGCTCTGTGGTCTTTGTGGCAACTACAATGGACATAAACGTGA
TGACTTAATTGGTGGAGATGGAAACTTCAAGTTTGATGTGGATGACTTTG
CTGAATCTTGGAGGGTGGAGTCCAATGAGTTCTGCAACAGACCTCAGAGA
AAGCCAGTGCCTGAACTGTGTCAAGGGACAGTCAAGGTAAAGCTCCGGGC
CCATCGAGAATGCCAAAAGCTCAAATCCTGGGAGTTTCAGACCTGCCACT
CGACTGTGGACTACGCCACTTTCTACCGGTCCTGTGTGACAGACATGTGT
GAATGTCCAGTCCATAAAAACTGTTATTGCGAGTCATTTTTGGCATATAC
CCGGGCCTGCCAGAGAGAGGGCATCAAAGTCCACTGGGAGCCTCAGCAGA
ATTGTGCAGCCACCCAGTGTAAGCATGGTGCTGTGTACGATACCTGTGGT
CCGGGATGTATCAAGACGTGTGACAACTGGAATGAAATTGGTCCATGCAA
CAAGCCGTGCGTTGCTGGGTGCCACTGTCCAGCAAACTTGGTCCTTCACA
AGGGAAGGTGCATCAAGCCAGTCCTTTGTCCCCAGCGG

In certain embodiments, the disclosure relates to heteromultimers that comprise at least one BMPER polypeptide, which includes fragments, functional variants, and modified forms thereof. Preferably, BMPER polypeptides for use in accordance with the disclosure (e.g., heteromultimers comprising a BMPER polypeptide and uses thereof) are soluble (e.g., an extracellular domain of BMPER). In other preferred embodiments, BMPER polypeptides for use in accordance with the disclosure bind to and/or inhibit (antagonize) activity (e.g., Smad signaling) of one or more TGF-beta superfamily ligands. In some embodiments, heteromultimers of the disclosure comprise at least one BMPER polypeptide that is at least 70%, 75%, 80%, 85%, 90%, 91%, 92%, 93%, 94%, 95%, 96%, 97%, 98%, 99%, or 100% identical to the amino acid sequence of SEQ ID NOs: 553 or 554. In some embodiments, heteromultimers of the disclosure comprise at least one BMPER polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 40-50 (e.g., amino acid residues 40, 41, 42, 43, 44, 45, 46, 47, 48, 49, or 50) of SEQ ID NO: 553, and ends at any one of amino acids 364-369 (e.g., amino acid residues 364, 365, 366, 367, 368, or 369) of SEQ ID NO: 553. In some embodiments, heteromultimers of the disclosure comprise at least one BMPER polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 370-386 (e.g., amino acid residues 370, 371, 372, 373, 374, 375, 376, 377, 378, 379, 380, 381, 382, 383, 284, 385, or 386) of SEQ ID NO: 553, and ends at any one of amino acids 682-685 (e.g., amino acid residues 682, 683, 684, or 685) of SEQ ID NO: 553. In some embodiments, heteromultimers of the disclosure comprise at least one BMPER polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 39-50 (e.g., amino acid residues 39, 40, 41, 42, 43, 44, 45, 46, 47, 48, 49, or 50) of SEQ ID NO: 553, and ends at any one of amino acids 682-685 (e.g., amino acid residues 682, 683, 684, or 685) of SEQ ID NO: 553. In some embodiments, heteromultimers of the disclosure comprise at least one BMPER polypeptide that is at least 70%, 75%, 80%, 85%, 90%, 91%, 92%, 93%, 94%, 95%, 96%, 97%, 98%, 99%, or 100% identical to amino acids of 39-364 of SEQ ID NO: 553. In some embodiments, heteromultimers of the disclosure comprise at least one BMPER polypeptide that is at least 70%, 75%, 80%, 85%, 90%, 91%, 92%, 93%, 94%, 95%, 96%, 97%, 98%, 99%, or 100% identical to amino acids of 39-369 of SEQ ID NO: 553. In some embodiments, heteromultimers of the disclosure comprise at least one BMPER polypeptide that is at least 70%, 75%, 80%, 85%, 90%, 91%, 92%, 93%, 94%, 95%, 96%, 97%, 98%, 99%, or 100% identical to amino acids of 39-682 of SEQ ID NO: 553. In some embodiments, heteromultimers of the disclosure comprise at least one BMPER polypeptide that is at least 70%, 75%, 80%, 85%, 90%, 91%, 92%, 93%, 94%, 95%, 96%, 97%, 98%, 99%, or 100% identical to amino acids of 39-685 of SEQ ID NO: 553. In some embodiments, heteromultimers of the disclosure comprise at least one BMPER polypeptide that is at least 70%, 75%, 80%, 85%, 90%, 91%, 92%, 93%, 94%, 95%, 96%, 97%, 98%, 99%, or 100% identical to amino acids of 50-364 of SEQ ID NO: 553. In some embodiments, heteromultimers of the disclosure comprise at least one BMPER polypeptide that is at least 70%, 75%, 80%, 85%, 90%, 91%, 92%, 93%, 94%, 95%, 96%, 97%, 98%, 99%, or 100% identical to amino acids of 50-369 of SEQ ID NO: 553. In some embodiments, heteromultimers of the disclosure comprise at least one BMPER polypeptide that is at least 70%, 75%, 80%, 85%, 90%, 91%, 92%, 93%, 94%, 95%, 96%, 97%, 98%, 99%, or 100% identical to amino acids of 50-682 of SEQ ID NO: 553. In some embodiments, heteromultimers of the disclosure comprise at least one BMPER polypeptide that is at least 70%, 75%, 80%, 85%, 90%, 91%, 92%, 93%, 94%, 95%, 96%, 97%, 98%, 99%, or 100% identical to amino acids of 50-685 of SEQ ID NO: 553. In some embodiments, heteromultimers of the disclosure comprise at least one BMPER polypeptide that is at least 70%, 75%, 80%, 85%, 90%, 91%, 92%, 93%, 94%, 95%, 96%, 97%, 98%, 99%, or 100% identical to amino acids of 370-682 of SEQ ID NO: 553. In some embodiments, heteromultimers of the disclosure comprise at least one BMPER polypeptide that is at least 70%, 75%, 80%, 85%, 90%, 91%, 92%, 93%, 94%, 95%, 96%, 97%, 98%, 99%, or 100% identical to amino acids of 370-685 of SEQ ID NO: 553. In some embodiments, heteromultimers of the disclosure comprise at least one BMPER polypeptide that is at least 70%, 75%, 80%, 85%, 90%, 91%, 92%, 93%, 94%, 95%, 96%, 97%, 98%, 99%, or 100% identical to amino acids of 386-682 of SEQ ID NO: 553. In some embodiments, heteromultimers of the disclosure comprise at least one BMPER polypeptide that is at least 70%, 75%, 80%, 85%, 90%, 91%, 92%, 93%, 94%, 95%, 96%, 97%, 98%, 99%, or 100% identical to amino acids of 386-685 of SEQ ID NO: 553. In some embodiments, heteromultimers of the disclosure comprise at least a BMPER protein, wherein the BMPER protein is a dimer comprising a first polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 39-50 (e.g., amino acid residues 39, 40, 41, 42, 43, 44, 45, 46, 47, 48, 49, or 50) of SEQ ID NO: 553, and ends at any one of amino acids 364-369 (e.g., amino acid residues 364, 365, 366, 367, 368, or 369) of SEQ ID NO: 553, and second polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 370-386 (e.g., amino acid residues 370, 371, 372, 373, 374, 375, 376, 377, 378, 379, 380, 381, 382, 383, 284, 385, or 386) of SEQ ID NO: 553, and ends at any one of amino acids 682-685 (e.g., amino acid residues 682, 683, 684, or 685) of SEQ ID NO: 553. In some embodiments, heteromultimers of the disclosure comprise at least one single chain ligand trap that comprises a first BMPER polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 39-50 (e.g., amino acid residues 39, 40, 41, 42, 43, 44, 45, 46, 47, 48, 49, or 50) of SEQ ID NO: 553, and ends at any one of amino acids 364-369 (e.g., amino acid residues 364, 365, 366, 367, 368, or 369) of SEQ ID NO: 553, and second BMPER polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 370-386 (e.g., amino acid residues 370, 371, 372, 373, 374, 375, 376, 377, 378, 379, 380, 381, 382, 383, 284, 385, or 386) of SEQ ID NO: 553, and ends at any one of amino acids 682-685 (e.g., amino acid residues 682, 683, 684, or 685) of SEQ ID NO: 553.

The term “RGM-B polypeptide” includes polypeptides comprising any naturally occurring RGM-B protein (encoded by RGMB or one of its nonhuman orthologs) as well as any variants thereof (including mutants, fragments, fusions, and peptidomimetic forms) that retain a useful activity.

A human RGM-B precursor protein sequence (NCBI Ref Seq NP_001012779.2) is as follows:

| (SEQ ID NO: 557) | |
|---|---|
| 1 | MIRKKRKRSA PPGPCRSHGP RPATAPAPPP SPEPTRPAWT GMGLRAAPSS AAAAAAEVEQ |
| 61 | RRSPGLCPPP LELLLLLLFS LGLLHAGDCQ QPAQCRIQKC TTDFVSLTSH LNSAVDGFDS |
| 121 | EFCKALRAYA GCTQRTSKAC RGNLVYHSAV LGISDLMSQR NCSKDGPTSS TNPEVTHDPC |
| 181 | NYHSHAGARE HRRGDQNPPS YLFCGLFGDP HLRTFKDNFQ TCKVEGAWPL IDNNYLSVQV |
| 241 | TNVPVVPGSS ATATNKITII FKAHHECTDQ KVYQAVTDDL PAAFVDGTTS GGDSDAKSLR |
| 301 | IVERESGHYV EMHARYIGTT VFVRQVGRYL TLAIRMPEDL AMSYEESQDL QLCVNGCPLS |
| 361 | ERIDDGQGQV SAILGHSLPR TSLVQAWPGY TLETANTQCH EKMPVKDIYF QSCVFDLLTT |
| 421 | GDANFTAAAH SALEDVEALH PRKERWHIFP SSGNGTPRGG SDLSVSLGLT CLILIVFL |

The signal peptide is indicated by single underline.

A processed RGM-B polypeptide sequence is as follows:

(SEQ ID NO: 558)

GDCQQPAQCRIQKCTTDFVSLTSHLNSAVDGFDSEFCKALRAYAGCTQRT
SKACRGNLVYHSAVLGISDLMSQRNCSKDGPTSSTNPEVTHDPCNYHSHA
GAREHRRGDQNPPSYLFCGLFGDPHLRTFKDNFQTCKVEGAWPLIDNNYL
SVQVTNVPVVPGSSATATNKITIIFKAHHECTDQKVYQAVTDDLPAAFVD
GTTSGGDSDAKSLRIVERESGHYVEMHARYIGTTVFVRQVGRYLTLAIRM
PEDLAMSYEESQDLQLCVNGCPLSERIDDGQGQVSAILGHSLPRTSLVQA
WPGYTLETANTQCHEKMPVKDIYFQSCVFDLLTTGDANFTAAAHSALEDV
EALHPRKERWHIFPSS

A nucleic acid sequence encoding unprocessed human RGM-B precursor protein is shown below (SEQ ID NO: 559), corresponding to nucleotides 403-1836 of NCBI Reference Sequence NM_001012761.2. The signal sequence is underlined.

(SEQ ID NO: 559)

ATGATAAGGAAGAAGAGGAAGCGAAGCGCGCCCCCCGGCCCATGCCGCAG
CCACGGGCCCAGACCCGCCACGGCGCCCGCGCCGCCGCCCTCGCCGGAGC
CCACGAGACCTGCATGGACGGGCATGGGCTTGAGAGCAGCACCTTCCAGC
GCCGCCGCTGCCGCCGCCGAGGTTGAGCAGCGCCGCAGCCCCGGGCTCTG
CCCCCCGCCGCTGGAGCTGCTGCTGCTGCTGCTGTTCAGCCTCGGGCTGC
TCCACGCAGGTGACTGCCAACAGCCAGCCCAATGTCGAATCCAGAAATGC
ACCACGGACTTCGTGTCCCTGACTTCTCACCTGAACTCTGCCGTTGACGG
CTTTGACTCTGAGTTTTGCAAGGCCTTGCGTGCCTATGCTGGCTGCACCC
AGCGAACTTCAAAAGCCTGCCGTGGCAACCTGGTATACCATTCTGCCGTG
TTGGGTATCAGTGACCTCATGAGCCAGAGGAATTGTTCCAAGGATGGACC
CACATCCTCTACCAACCCCGAAGTGACCCATGATCCTTGCAACTATCACA
GCCACGCTGGAGCCAGGGAACACAGGAGAGGGGACCAGAACCCTCCCAGT
TACCTTTTTTGTGGCTTGTTTGGAGATCCTCACCTCAGAACTTTCAAGGA
TAACTTCCAAACATGCAAAGTAGAAGGGGCCTGGCCACTCATAGATAATA
ATTATCTTTCAGTTCAAGTGACAAACGTACCTGTGGTCCCTGGATCCAGT
GCTACTGCTACAAATAAGATCACTATTATCTTCAAAGCCCACCATGAGTG
TACAGATCAGAAAGTCTACCAAGCTGTGACAGATGACCTGCCGGCCGCCT
TTGTGGATGGCACCACCAGTGGTGGGGACAGCGATGCCAAGAGCCTGCGT
ATCGTGGAAAGGGAGAGTGGCCACTATGTGGAGATGCACGCCCGCTATAT
AGGGACCACAGTGTTTGTGCGGCAGGTGGGTCGCTACCTGACCCTTGCCA
TCCGTATGCCTGAAGACCTGGCCATGTCCTACGAGGAGAGCCAGGACCTG
CAGCTGTGCGTGAACGGCTGCCCCCTGAGTGAACGCATCGATGACGGGCA
GGGCCAGGTGTCTGCCATCCTGGGACACAGCCTGCCTCGCACCTCCTTGG
TGCAGGCCTGGCCTGGCTACACACTGGAGACTGCCAACACTCAATGCCAT
GAGAAGATGCCAGTGAAGGACATCTATTTCCAGTCCTGTGTCTTCGACCT
GCTCACCACTGGTGATGCCAACTTTACTGCCGCAGCCCACAGTGCCTTGG
AGGATGTGGAGGCCCTGCACCCAAGGAAGGAACGCTGGCACATTTTCCCC
AGCAGTGGCAATGGGACTCCCCGTGGAGGCAGTGATTTGTCTGTCAGTCT
AGGACTCACCTGCTTGATCCTTATCGTGTTTTTG

A nucleic acid sequence encoding a processed RGM-B is shown below (SEQ ID NO: 560):

(SEQ ID NO: 560)

GGTGACTGCCAACAGCCAGCCCAATGTCGAATCCAGAAATGCACCACGGA
CTTCGTGTCCCTGACTTCTCACCTGAACTCTGCCGTTGACGGCTTTGACT
CTGAGTTTTGCAAGGCCTTGCGTGCCTATGCTGGCTGCACCCAGCGAACT
TCAAAAGCCTGCCGTGGCAACCTGGTATACCATTCTGCCGTGTTGGGTAT
CAGTGACCTCATGAGCCAGAGGAATTGTTCCAAGGATGGACCCACATCCT
CTACCAACCCCGAAGTGACCCATGATCCTTGCAACTATCACAGCCACGCT
GGAGCCAGGGAACACAGGAGAGGGGACCAGAACCCTCCCAGTTACCTTTT
TTGTGGCTTGTTTGGAGATCCTCACCTCAGAACTTTCAAGGATAACTTCC
AAACATGCAAAGTAGAAGGGGCCTGGCCACTCATAGATAATAATTATCTT
TCAGTTCAAGTGACAAACGTACCTGTGGTCCCTGGATCCAGTGCTACTGC
TACAAATAAGATCACTATTATCTTCAAAGCCCACCATGAGTGTACAGATC
AGAAAGTCTACCAAGCTGTGACAGATGACCTGCCGGCCGCCTTTGTGGAT
GGCACCACCAGTGGTGGGGACAGCGATGCCAAGAGCCTGCGTATCGTGGA
AAGGGAGAGTGGCCACTATGTGGAGATGCACGCCCGCTATATAGGGACCA
CAGTGTTTGTGCGGCAGGTGGGTCGCTACCTGACCCTTGCCATCCGTATG
CCTGAAGACCTGGCCATGTCCTACGAGGAGAGCCAGGACCTGCAGCTGTG
CGTGAACGGCTGCCCCCTGAGTGAACGCATCGATGACGGGCAGGGCCAGG
TGTCTGCCATCCTGGGACACAGCCTGCCTCGCACCTCCTTGGTGCAGGCC
TGGCCTGGCTACACACTGGAGACTGCCAACACTCAATGCCATGAGAAGAT
GCCAGTGAAGGACATCTATTTCCAGTCCTGTGTCTTCGACCTGCTCACCA
CTGGTGATGCCAACTTTACTGCCGCAGCCCACAGTGCCTTGGAGGATGTG
GAGGCCCTGCACCCAAGGAAGGAACGCTGGCACATTTTCCCCAGCAGT

In certain embodiments, the disclosure relates to heteromultimers that comprise at least one RGM-B polypeptide, which includes fragments, functional variants, and modified forms thereof. Preferably, RGM-B polypeptides for use in accordance with the disclosure (e.g., heteromultimers comprising a RGM-B polypeptide and uses thereof) are soluble (e.g., an extracellular domain of RGM-B). In other preferred embodiments, RGM-B polypeptides for use in accordance with the disclosure bind to and/or inhibit (antagonize) activity (e.g., Smad signaling) of one or more TGF-beta superfamily ligands. In some embodiments, heteromultimers of the disclosure comprise at least one RGM-B polypeptide that is at least 70%, 75%, 80%, 85%, 90%, 91%, 92%, 93%, 94%, 95%, 96%, 97%, 98%, 99%, or 100% identical to the amino acid sequence of SEQ ID NOs: 557 or 558. In some embodiments, heteromultimers of the disclosure comprise at least one RGM-B polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 1-87 (e.g., amino acid residues 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, or 87) of SEQ ID NO: 557, and ends at any one of amino acids 452-478 (e.g., amino acid residues 452, 453, 454, 455, 456, 457, 458, 459, 460, 461, 462, 463, 464, 465, 466, 467, 468, 469, 470, 471, 472, 473, 474, 475, 476, 477, or 478) of SEQ ID NO: 557. In some embodiments, heteromultimers of the disclosure comprise at least one RGM-B polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 210-222 (e.g., amino acid residues 210, 211, 212, 213, 214, 215, 216, 217, 218, 219, 220, 221, or 222) of SEQ ID NO: 557, and ends at any one of amino acids 413-452 (e.g., amino acid residues 413, 414, 415, 416, 417, 418, 419, 420, 421, 422, 423, 424, 425, 426, 427, 428, 429, 430, 431, 432, 433, 434, 435, 435, 436, 437, 438, 439, 440, 441, 442, 443, 444, 445, 446, 447, 448, 449, 450, 451, or 452) of SEQ ID NO: 557. In some embodiments, heteromultimers of the disclosure comprise at least one RGM-B polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 87-95 (e.g., amino acid residues 87, 88, 89, 90, 91, 92, 93, 94 or 95) of SEQ ID NO: 557, and ends at any one of amino acids 204-209 (e.g., amino acid residues 204, 205, 206, 207, 208, or 209) of SEQ ID NO: 557. In some embodiments, heteromultimers of the disclosure comprise of at least one RGM-B polypeptide that is at least 70%, 75%, 80%, 85%, 90%, 91%, 92%, 93%, 94%, 95%, 96%, 97%, 98%, 99%, or 100% identical to amino acids of 1-452 of SEQ ID NO: 557. In some embodiments, heteromultimers of the disclosure comprise of at least one RGM-B polypeptide that is at least 70%, 75%, 80%, 85%, 90%, 91%, 92%, 93%, 94%, 95%, 96%, 97%, 98%, 99%, or 100% identical to amino acids of 87-204 of SEQ ID NO: 557. In some embodiments, heteromultimers of the disclosure comprise of at least one RGM-B polypeptide that is at least 70%, 75%, 80%, 85%, 90%, 91%, 92%, 93%, 94%, 95%, 96%, 97%, 98%, 99%, or 100% identical to amino acids of 87-209 of SEQ ID NO: 557. In some embodiments, heteromultimers of the disclosure comprise of at least one RGM-B polypeptide that is at least 70%, 75%, 80%, 85%, 90%, 91%, 92%, 93%, 94%, 95%, 96%, 97%, 98%, 99%, or 100% identical to amino acids of 95-204 of SEQ ID NO: 557. In some embodiments, heteromultimers of the disclosure comprise of at least one RGM-B polypeptide that is at least 70%, 75%, 80%, 85%, 90%, 91%, 92%, 93%, 94%, 95%, 96%, 97%, 98%, 99%, or 100% identical to amino acids of 95-209 of SEQ ID NO: 557. In some embodiments, heteromultimers of the disclosure comprise of at least one RGM-B polypeptide that is at least 70%, 75%, 80%, 85%, 90%, 91%, 92%, 93%, 94%, 95%, 96%, 97%, 98%, 99%, or 100% identical to amino acids of 210-413 of SEQ ID NO: 557. In some embodiments, heteromultimers of the disclosure comprise of at least one RGM-B polypeptide that is at least 70%, 75%, 80%, 85%, 90%, 91%, 92%, 93%, 94%, 95%, 96%, 97%, 98%, 99%, or 100% identical to amino acids of 210-452 of SEQ ID NO: 557. In some embodiments, heteromultimers of the disclosure comprise of at least one RGM-B polypeptide that is at least 70%, 75%, 80%, 85%, 90%, 91%, 92%, 93%, 94%, 95%, 96%, 97%, 98%, 99%, or 100% identical to amino acids of 222-413 of SEQ ID NO: 557. In some embodiments, heteromultimers of the disclosure comprise of at least one RGM-B polypeptide that is at least 70%, 75%, 80%, 85%, 90%, 91%, 92%, 93%, 94%, 95%, 96%, 97%, 98%, 99%, or 100% identical to amino acids of 222-452 of SEQ ID NO: 557. In some embodiments, heteromultimers of the disclosure comprise of at least one RGM-B polypeptide that is at least 70%, 75%, 80%, 85%, 90%, 91%, 92%, 93%, 94%, 95%, 96%, 97%, 98%, 99%, or 100% identical to amino acids of 87-413 of SEQ ID NO: 557. In some embodiments, heteromultimers of the disclosure comprise of at least one RGM-B polypeptide that is at least 70%, 75%, 80%, 85%, 90%, 91%, 92%, 93%, 94%, 95%, 96%, 97%, 98%, 99%, or 100% identical to amino acids of 87-452 of SEQ ID NO: 557. In some embodiments, heteromultimers of the disclosure comprise of at least one RGM-B polypeptide that is at least 70%, 75%, 80%, 85%, 90%, 91%, 92%, 93%, 94%, 95%, 96%, 97%, 98%, 99%, or 100% identical to amino acids of 95-413 of SEQ ID NO: 557. In some embodiments, heteromultimers of the disclosure comprise of at least one RGM-B polypeptide that is at least 70%, 75%, 80%, 85%, 90%, 91%, 92%, 93%, 94%, 95%, 96%, 97%, 98%, 99%, or 100% identical to amino acids of 95-452 of SEQ ID NO: 557. In some embodiments, heteromultimers of the disclosure comprise at least a RGM-B protein, wherein the RGM-B protein is a dimer comprising a first polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 87-95 (e.g., amino acid residues 87, 88, 89, 90, 91, 92, 93, 94 or 95) of SEQ ID NO: 557, and ends at any one of amino acids 204-209 (e.g., amino acid residues 204, 205, 206, 207, 208, or 209) of SEQ ID NO: 557, and second polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 210-222 (e.g., amino acid residues 210, 211, 212, 213, 214, 215, 216, 217, 218, 219, 220, 221, or 222) of SEQ ID NO: 557, and ends at any one of amino acids 413-452 (e.g., amino acid residues 413, 414, 415, 416, 417, 418, 419, 420, 421, 422, 423, 424, 425, 426, 427, 428, 429, 430, 431, 432, 433, 434, 435, 435, 436, 437, 438, 439, 440, 441, 442, 443, 444, 445, 446, 447, 448, 449, 450, 451, or 452) of SEQ ID NO: 557. In some embodiments, heteromultimers of the disclosure comprise at least one single chain ligand trap that comprises a first RGM-B polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 87-95 (e.g., amino acid residues 87, 88, 89, 90, 91, 92, 93, 94 or 95) of SEQ ID NO: 557, and ends at any one of amino acids 204-209 (e.g., amino acid residues 204, 205, 206, 207, 208, or 209) of SEQ ID NO: 557, and second RGM-B polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 210-222 (e.g., amino acid residues 210, 211, 212, 213, 214, 215, 216, 217, 218, 219, 220, 221, or 222) of SEQ ID NO: 557, and ends at any one of amino acids 413-452 (e.g., amino acid residues 413, 414, 415, 416, 417, 418, 419, 420, 421, 422, 423, 424, 425, 426, 427, 428, 429, 430, 431, 432, 433, 434, 435, 435, 436, 437, 438, 439, 440, 441, 442, 443, 444, 445, 446, 447, 448, 449, 450, 451, or 452) of SEQ ID NO: 557. In some embodiments, heteromultimers of the disclosure comprise at least one RGM-B polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 87-89 (e.g., amino acid residues 87, 88, or 89) of SEQ ID NO: 557, and ends at any one of amino acids 471-478 (e.g., amino acid residues 471, 472, 473, 474, 475, 476, 477, or 478) of SEQ ID NO: 557. In some embodiments, heteromultimers of the disclosure comprise at least one RGM-B polypeptide that is at least 70%, 75%, 80%, 85%, 90%, 91%, 92%, 93%, 94%, 95%, 96%, 97%, 98%, 99%, or 100% identical to amino acids 87-478 of SEQ ID NO: 557. In some embodiments, heteromultimers of the disclosure comprise at least one RGM-B polypeptide that is at least 70%, 75%, 80%, 85%, 90%, 91%, 92%, 93%, 94%, 95%, 96%, 97%, 98%, 99%, or 100% identical to amino acids 89-471 of SEQ ID NO: 557.

The term “RGM-A polypeptide” includes polypeptides comprising any naturally occurring RGM-A protein (encoded by RGMA or one of its nonhuman orthologs) as well as any variants thereof (including mutants, fragments, fusions, and peptidomimetic forms) that retain a useful activity.

A human RGM-A isoform 1 precursor protein sequence (NCBI Ref Seq NP_001159755.1) is as follows:

| (SEQ ID NO: 561) | |
|---|---|
| 1 | MGGLGPRRAG TSRERLVVTG RAGWMGMGRG AGRSALGFWP TLAFLLCSFP AATSPCKILK |
| 61 | CNSEFWSATS GSHAPASDDT PEFCAALRSY ALCTRRTART CRGDLAYHSA VHGIEDLMSQ |
| 121 | HNCSKDGPTS QPRLRTLPPA GDSQERSDSP EICHYEKSFH KHSATPNYTH CGLFGDPHLR |
| 181 | TFTDRFQTCK VQGAWPLIDN NYLNVQVTNT PVLPGSAATA TSKLTIIFKN FQECVDQKVY |
| 241 | QAEMDELPAA FVDGSKNGGD KHGANSLKIT EKVSGQHVEI QAKYIGTTIV VRQVGRYLTF |
| 301 | AVRMPEEVVN AVEDWDSQGL YLCLRGCPLN QQIDFQAFHT NAEGTGARRL AAASPAPTAP |
| 361 | ETFPYETAVA KCKEKLPVED LYYQACVFDL LTTGDVNFTL AAYYALEDVK MLHSNKDKLH |
| 421 | LYERTRDLPG RAAAGLPLAP RPLLGALVPL LALLPVFC |

The signal peptide is indicated by solid underline.

A processed RGM-A isoform 1 polypeptide sequence is as follows:

(SEQ ID NO: 562)

CKILKCNSEFWSATSGSHAPASDDTPEFCAALRSYALCTRRTARTCRGDL
AYHSAVHGIEDLMSQHNCSKDGPTSQPRLRTLPPAGDSQERSDSPEICHY
EKSFHKHSATPNYTHCGLFGDPHLRTFTDRFQTCKVQGAWPLIDNNYLNV
QVTNTPVLPGSAATATSKLTIIFKNFQECVDQKVYQAEMDELPAAFVDGS
KNGGDKHGANSLKITEKVSGQHVEIQAKYIGTTIVVRQVGRYLTFAVRMP
EEVVNAVEDWDSQGLYLCLRGCPLNQQIDFQAFHTNAEGTGARRLAAASP
APTAPETFPYETAVAKCKEKLPVEDLYYQACVFDLLTTGDVNFTLAAYYA
LEDVKMLHS

A nucleic acid sequence encoding unprocessed human RGM-A isoform 1 precursor protein is shown below (SEQ ID NO: 563), corresponding to nucleotides 232-1605 of NCBI Reference Sequence NM_001166283.1. The signal sequence is underlined.

(SEQ ID NO: 563)

ATGGGTGGCCTGGGGCCACGACGGGCGGGAACCTCGAGGGAGAGGCTAGT
GGTAACAGGCCGAGCTGGATGGATGGGTATGGGGAGAGGGGCAGGACGTT
CAGCCCTGGGATTCTGGCCGACCCTCGCCTTCCTTCTCTGCAGCTTCCCC
GCAGCCACCTCCCCGTGCAAGATCCTCAAGTGCAACTCTGAGTTCTGGAG
CGCCACGTCGGGCAGCCACGCCCCAGCCTCAGACGACACCCCCGAGTTCT
GTGCAGCCTTGCGCAGCTACGCCCTGTGCACGCGGCGGACGGCCCGCACC
TGCCGGGGTGACCTGGCCTACCACTCGGCCGTCCATGGCATAGAGGACCT
CATGAGCCAGCACAACTGCTCCAAGGATGGCCCCACCTCGCAGCCACGCC
TGCGCACGCTCCCACCGGCCGGAGACAGCCAGGAGCGCTCGGACAGCCCC
GAGATCTGCCATTACGAGAAGAGCTTTCACAAGCACTCGGCCACCCCCAA
CTACACGCACTGTGGCCTCTTCGGGGACCCACACCTCAGGACTTTCACCG
ACCGCTTCCAGACCTGCAAGGTGCAGGGCGCCTGGCCGCTCATCGACAAT
AATTACCTGAACGTGCAGGTCACCAACACGCCTGTGCTGCCCGGCTCAGC
GGCCACTGCCACCAGCAAGCTCACCATCATCTTCAAGAACTTCCAGGAGT
GTGTGGACCAGAAGGTGTACCAGGCTGAGATGGACGAGCTCCCGGCCGCC
TTCGTGGATGGCTCTAAGAACGGTGGGGACAAGCACGGGGCCAACAGCCT
GAAGATCACTGAGAAGGTGTCAGGCCAGCACGTGGAGATCCAGGCCAAGT
ACATCGGCACCACCATCGTGGTGCGCCAGGTGGGCCGCTACCTGACCTTT
GCCGTCCGCATGCCAGAGGAAGTGGTCAATGCTGTGGAGGACTGGGACAG
CCAGGGTCTCTACCTCTGCCTGCGGGGCTGCCCCCTCAACCAGCAGATCG
ACTTCCAGGCCTTCCACACCAATGCTGAGGGCACCGGTGCCCGCAGGCTG
GCAGCCGCCAGCCCTGCACCCACAGCCCCCGAGACCTTCCCATACGAGAC
AGCCGTGGCCAAGTGCAAGGAGAAGCTGCCGGTGGAGGACCTGTACTACC
AGGCCTGCGTCTTCGACCTCCTCACCACGGGCGACGTGAACTTCACACTG
GCCGCCTACTACGCGTTGGAGGATGTCAAGATGCTCCACTCCAACAAAGA
CAAACTGCACCTGTATGAGAGGACTCGGGACCTGCCAGGCAGGGCGGCTG
CGGGGCTGCCCCTGGCCCCCCGGCCCCTCCTGGGCGCCCTCGTCCCGCTC
CTGGCCCTGCTCCCTGTGTTCTGC

A nucleic acid sequence encoding a processed RGM-A isoform 1 is shown below (SEQ ID NO: 564):

(SEQ ID NO: 564)

TGCAAGATCCTCAAGTGCAACTCTGAGTTCTGGAGCGCCACGTCGGGCAG
CCACGCCCCAGCCTCAGACGACACCCCCGAGTTCTGTGCAGCCTTGCGCA
GCTACGCCCTGTGCACGCGGCGGACGGCCCGCACCTGCCGGGGTGACCTG
GCCTACCACTCGGCCGTCCATGGCATAGAGGACCTCATGAGCCAGCACAA
CTGCTCCAAGGATGGCCCCACCTCGCAGCCACGCCTGCGCACGCTCCCAC
CGGCCGGAGACAGCCAGGAGCGCTCGGACAGCCCCGAGATCTGCCATTAC
GAGAAGAGCTTTCACAAGCACTCGGCCACCCCCAACTACACGCACTGTGG
CCTCTTCGGGGACCCACACCTCAGGACTTTCACCGACCGCTTCCAGACCT
GCAAGGTGCAGGGCGCCTGGCCGCTCATCGACAATAATTACCTGAACGTG
CAGGTCACCAACACGCCTGTGCTGCCCGGCTCAGCGGCCACTGCCACCAG
CAAGCTCACCATCATCTTCAAGAACTTCCAGGAGTGTGTGGACCAGAAGG
TGTACCAGGCTGAGATGGACGAGCTCCCGGCCGCCTTCGTGGATGGCTCT
AAGAACGGTGGGGACAAGCACGGGGCCAACAGCCTGAAGATCACTGAGAA
GGTGTCAGGCCAGCACGTGGAGATCCAGGCCAAGTACATCGGCACCACCA
TCGTGGTGCGCCAGGTGGGCCGCTACCTGACCTTTGCCGTCCGCATGCCA
GAGGAAGTGGTCAATGCTGTGGAGGACTGGGACAGCCAGGGTCTCTACCT
CTGCCTGCGGGGCTGCCCCCTCAACCAGCAGATCGACTTCCAGGCCTTCC
ACACCAATGCTGAGGGCACCGGTGCCCGCAGGCTGGCAGCCGCCAGCCCT
GCACCCACAGCCCCCGAGACCTTCCCATACGAGACAGCCGTGGCCAAGTG
CAAGGAGAAGCTGCCGGTGGAGGACCTGTACTACCAGGCCTGCGTCTTCG
ACCTCCTCACCACGGGCGACGTGAACTTCACACTGGCCGCCTACTACGCG
TTGGAGGATGTCAAGATGCTCCACTCC

A human RGM-A isoform 2 precursor protein sequence (NCBI Ref Seq NP_001159758.1) is as follows:

| (SEQ ID NO: 565) | |
|---|---|
| 1 | MGMGRGAGRS ALGFWPTLAF LLCSFPAATS PCKILKCNSE FWSATSGSHA PASDDTPEFC |
| 61 | AALRSYALCT RRTARTCRGD LAYHSAVHGI EDLMSQHNCS KDGPTSQPRL RTLPPAGDSQ |
| 121 | ERSDSPEICH YEKSFHKHSA TPNYTHCGLF GDPHLRTFTD RFQTCKVQGA WPLIDNNYLN |
| 181 | VQVTNTPVLP GSAATATSKL TIIFKNFQEC VDQKVYQAEM DELPAAFVDG SKNGGDKHGA |
| 241 | NSLKITEKVS GQHVEIQAKY IGTTIVVRQV GRYLTFAVRM PEEVVNAVED WDSQGLYLCL |
| 301 | RGCPLNQQID FQAFHTNAEG TGARRLAAAS PAPTAPETFP YETAVAKCKE KLPVEDLYYQ |
| 361 | ACVFDLLTTG DVNFTLAAYY ALEDVKMLHS NKDKLHLYER TRDLPGRAAA GLPLAPRPLL |
| 421 | GALVPLLALL PVFC |

The signal peptide is indicated by solid underline.

A mature RGM-A isoform 2 sequence is as follows: CKILKCNSEFWSATSGSHAPASDDTPEFCAALRSYALCTRRTARTCRGDLAYHSAVHGIEDLMSQHNCSKDGPTS QPRLRTLPPAGDSQERSDSPEICHYEKSFHKHSATPNYTHCGLFGDPHLRTFTDRFQTCKVQGAWPLIDNNYLNV QVTNTPVLPGSAATATSKLTIIFKNFQECVDQKVYQAEMDELPAAFVDGSKNGGDKHGANSLKITEKVSGQHVEI QAKYIGTTIVVRQVGRYLTFAVRMPEEVVNAVEDWDSQGLYLCLRGCPLNQQIDFQAFHTNAEGTGARRLAAASP APTAPETFPYETAVAKCKEKLPVEDLYYQACVFDLLTTGDVNFTLAAYYALEDVKMLHS (SEQ ID NO: 566)

A nucleic acid sequence encoding unprocessed human RGM-A isoform 2 precursor protein is shown below (SEQ ID NO: 567), corresponding to nucleotides 164-1465 of NCBI Reference Sequence NM_001166286.1. The signal sequence is underlined.

(SEQ ID NO: 567)

ATGGGTATGGGGAGAGGGGCAGGACGTTCAGCCCTGGGATTCTGGCCGAC
CCTCGCCTTCCTTCTCTGCAGCTTCCCCGCAGCCACCTCCCCGTGCAAGA
TCCTCAAGTGCAACTCTGAGTTCTGGAGCGCCACGTCGGGCAGCCACGCC
CCAGCCTCAGACGACACCCCCGAGTTCTGTGCAGCCTTGCGCAGCTACGC
CCTGTGCACGCGGCGGACGGCCCGCACCTGCCGGGGTGACCTGGCCTACC
ACTCGGCCGTCCATGGCATAGAGGACCTCATGAGCCAGCACAACTGCTCC
AAGGATGGCCCCACCTCGCAGCCACGCCTGCGCACGCTCCCACCGGCCGG
AGACAGCCAGGAGCGCTCGGACAGCCCCGAGATCTGCCATTACGAGAAGA
GCTTTCACAAGCACTCGGCCACCCCCAACTACACGCACTGTGGCCTCTTC
GGGGACCCACACCTCAGGACTTTCACCGACCGCTTCCAGACCTGCAAGGT
GCAGGGCGCCTGGCCGCTCATCGACAATAATTACCTGAACGTGCAGGTCA
CCAACACGCCTGTGCTGCCCGGCTCAGCGGCCACTGCCACCAGCAAGCTC
ACCATCATCTTCAAGAACTTCCAGGAGTGTGTGGACCAGAAGGTGTACCA
GGCTGAGATGGACGAGCTCCCGGCCGCCTTCGTGGATGGCTCTAAGAACG
GTGGGGACAAGCACGGGGCCAACAGCCTGAAGATCACTGAGAAGGTGTCA
GGCCAGCACGTGGAGATCCAGGCCAAGTACATCGGCACCACCATCGTGGT
GCGCCAGGTGGGCCGCTACCTGACCTTTGCCGTCCGCATGCCAGAGGAAG
TGGTCAATGCTGTGGAGGACTGGGACAGCCAGGGTCTCTACCTCTGCCTG
CGGGGCTGCCCCCTCAACCAGCAGATCGACTTCCAGGCCTTCCACACCAA
TGCTGAGGGCACCGGTGCCCGCAGGCTGGCAGCCGCCAGCCCTGCACCCA
CAGCCCCCGAGACCTTCCCATACGAGACAGCCGTGGCCAAGTGCAAGGAG
AAGCTGCCGGTGGAGGACCTGTACTACCAGGCCTGCGTCTTCGACCTCCT
CACCACGGGCGACGTGAACTTCACACTGGCCGCCTACTACGCGTTGGAGG
ATGTCAAGATGCTCCACTCCAACAAAGACAAACTGCACCTGTATGAGAGG
ACTCGGGACCTGCCAGGCAGGGCGGCTGCGGGGCTGCCCCTGGCCCCCCG
GCCCCTCCTGGGCGCCCTCGTCCCGCTCCTGGCCCTGCTCCCTGTGTTCT
GC

A nucleic acid sequence encoding a processed RGM-A isoform 2 is shown below (SEQ ID NO: 568):

(SEQ ID NO: 568)

TGCAAGATCCTCAAGTGCAACTCTGAGTTCTGGAGCGCCACGTCGGGCAG
CCACGCCCCAGCCTCAGACGACACCCCCGAGTTCTGTGCAGCCTTGCGCA
GCTACGCCCTGTGCACGCGGCGGACGGCCCGCACCTGCCGGGGTGACCTG
GCCTACCACTCGGCCGTCCATGGCATAGAGGACCTCATGAGCCAGCACAA
CTGCTCCAAGGATGGCCCCACCTCGCAGCCACGCCTGCGCACGCTCCCAC
CGGCCGGAGACAGCCAGGAGCGCTCGGACAGCCCCGAGATCTGCCATTAC
GAGAAGAGCTTTCACAAGCACTCGGCCACCCCCAACTACACGCACTGTGG
CCTCTTCGGGGACCCACACCTCAGGACTTTCACCGACCGCTTCCAGACCT
GCAAGGTGCAGGGCGCCTGGCCGCTCATCGACAATAATTACCTGAACGTG
CAGGTCACCAACACGCCTGTGCTGCCCGGCTCAGCGGCCACTGCCACCAG
CAAGCTCACCATCATCTTCAAGAACTTCCAGGAGTGTGTGGACCAGAAGG
TGTACCAGGCTGAGATGGACGAGCTCCCGGCCGCCTTCGTGGATGGCTCT
AAGAACGGTGGGGACAAGCACGGGGCCAACAGCCTGAAGATCACTGAGAA
GGTGTCAGGCCAGCACGTGGAGATCCAGGCCAAGTACATCGGCACCACCA
TCGTGGTGCGCCAGGTGGGCCGCTACCTGACCTTTGCCGTCCGCATGCCA
GAGGAAGTGGTCAATGCTGTGGAGGACTGGGACAGCCAGGGTCTCTACCT
CTGCCTGCGGGGCTGCCCCCTCAACCAGCAGATCGACTTCCAGGCCTTCC
ACACCAATGCTGAGGGCACCGGTGCCCGCAGGCTGGCAGCCGCCAGCCCT
GCACCCACAGCCCCCGAGACCTTCCCATACGAGACAGCCGTGGCCAAGTG
CAAGGAGAAGCTGCCGGTGGAGGACCTGTACTACCAGGCCTGCGTCTTCG
ACCTCCTCACCACGGGCGACGTGAACTTCACACTGGCCGCCTACTACGCG
TTGGAGGATGTCAAGATGCTCCACTCC

A human RGM-A isoform 3 precursor protein sequence (NCBI Ref Seq NP_064596.2) is as follows:

| (SEQ ID NO: 569) | |
|---|---|
| 1 | MQPPRERLVV TGRAGWMGMG RGAGRSALGF WPTLAFLLCS FPAATSPCKI LKCNSEFWSA |
| 61 | TSGSHAPASD DTPEFCAALR SYALCTRRTA RTCRGDLAYH SAVHGIEDLM SQHNCSKDGP |
| 121 | TSQPRLRTLP PAGDSQERSD SPEICHYEKS FHKHSATPNY THCGLFGDPH LRTFTDRFQT |
| 181 | CKVQGAWPLI DNNYLNVQVT NTPVLPGSAA TATSKLTIIF KNFQECVDQK VYQAEMDELP |
| 241 | AAFVDGSKNG GDKHGANSLK ITEKVSGQHV EIQAKYIGTT IVVRQVGRYL TFAVRMPEEV |
| 301 | VNAVEDWDSQ GLYLCLRGCP LNQQIDFQAF HTNAEGTGAR RLAAASPAPT APETFPYETA |
| 361 | VAKCKEKLPV EDLYYQACVF DLLTTGDVNF TLAAYYALED VKMLHSNKDK LHLYERTRDL |
| 421 | PGRAAAGLPL APRPLLGALV PLLALLPVFC |

The signal peptide is indicated by solid underline.

A mature RGM-A isoform 3 sequence is as follows:

(SEQ ID NO: 570)

CKILKCNSEFWSATSGSHAPASDDTPEFCAALRSYALCTRRTARTCRGDL
AYHSAVHGIEDLMSQHNCSKDGPTSQPRLRTLPPAGDSQERSDSPEICHY
EKSFHKHSATPNYTHCGLFGDPHLRTFTDRFQTCKVQGAWPLIDNNYLNV
QVTNTPVLPGSAATATSKLTIIFKNFQECVDQKVYQAEMDELPAAFVDGS
KNGGDKHGANSLKITEKVSGQHVEIQAKYIGTTIVVRQVGRYLTFAVRMP
EEVVNAVEDWDSQGLYLCLRGCPLNQQIDFQAFHTNAEGTGARRLAAASP
APTAPETFPYETAVAKCKEKLPVEDLYYQACVFDLLTTGDVNFTLAAYYA
LEDVKMLHS

A nucleic acid sequence encoding unprocessed RGM-A isoform 3 precursor protein is shown below (SEQ ID NO: 571), corresponding to nucleotides 283-1632 of NCBI Reference Sequence NM_020211.2. The signal sequence is underlined.

(SEQ ID NO: 571)

ATGCAGCCGCCAAGGGAGAGGCTAGTGGTAACAGGCCGAGCTGGATGGAT
GGGTATGGGGAGAGGGGCAGGACGTTCAGCCCTGGGATTCTGGCCGACCC
TCGCCTTCCTTCTCTGCAGCTTCCCCGCAGCCACCTCCCCGTGCAAGATC
CTCAAGTGCAACTCTGAGTTCTGGAGCGCCACGTCGGGCAGCCACGCCCC
AGCCTCAGACGACACCCCCGAGTTCTGTGCAGCCTTGCGCAGCTACGCCC
TGTGCACGCGGCGGACGGCCCGCACCTGCCGGGGTGACCTGGCCTACCAC
TCGGCCGTCCATGGCATAGAGGACCTCATGAGCCAGCACAACTGCTCCAA
GGATGGCCCCACCTCGCAGCCACGCCTGCGCACGCTCCCACCGGCCGGAG
ACAGCCAGGAGCGCTCGGACAGCCCCGAGATCTGCCATTACGAGAAGAGC
TTTCACAAGCACTCGGCCACCCCCAACTACACGCACTGTGGCCTCTTCGG
GGACCCACACCTCAGGACTTTCACCGACCGCTTCCAGACCTGCAAGGTGC
AGGGCGCCTGGCCGCTCATCGACAATAATTACCTGAACGTGCAGGTCACC
AACACGCCTGTGCTGCCCGGCTCAGCGGCCACTGCCACCAGCAAGCTCAC
CATCATCTTCAAGAACTTCCAGGAGTGTGTGGACCAGAAGGTGTACCAGG
CTGAGATGGACGAGCTCCCGGCCGCCTTCGTGGATGGCTCTAAGAACGGT
GGGGACAAGCACGGGGCCAACAGCCTGAAGATCACTGAGAAGGTGTCAGG
CCAGCACGTGGAGATCCAGGCCAAGTACATCGGCACCACCATCGTGGTGC
GCCAGGTGGGCCGCTACCTGACCTTTGCCGTCCGCATGCCAGAGGAAGTG
GTCAATGCTGTGGAGGACTGGGACAGCCAGGGTCTCTACCTCTGCCTGCG
GGGCTGCCCCCTCAACCAGCAGATCGACTTCCAGGCCTTCCACACCAATG
CTGAGGGCACCGGTGCCCGCAGGCTGGCAGCCGCCAGCCCTGCACCCACA
GCCCCCGAGACCTTCCCATACGAGACAGCCGTGGCCAAGTGCAAGGAGAA
GCTGCCGGTGGAGGACCTGTACTACCAGGCCTGCGTCTTCGACCTCCTCA
CCACGGGCGACGTGAACTTCACACTGGCCGCCTACTACGCGTTGGAGGAT
GTCAAGATGCTCCACTCCAACAAAGACAAACTGCACCTGTATGAGAGGAC
TCGGGACCTGCCAGGCAGGGCGGCTGCGGGGCTGCCCCTGGCCCCCCGGC
CCCTCCTGGGCGCCCTCGTCCCGCTCCTGGCCCTGCTCCCTGTGTTCTGC

A nucleic acid sequence encoding processed RGM-A isoform 3 is shown below (SEQ ID NO: 572):

(SEQ ID NO: 572)

TGCAAGATCCTCAAGTGCAACTCTGAGTTCTGGAGCGCCACGTCGGGCAG
CCACGCCCCAGCCTCAGACGACACCCCCGAGTTCTGTGCAGCCTTGCGCA
GCTACGCCCTGTGCACGCGGCGGACGGCCCGCACCTGCCGGGGTGACCTG
GCCTACCACTCGGCCGTCCATGGCATAGAGGACCTCATGAGCCAGCACAA
CTGCTCCAAGGATGGCCCCACCTCGCAGCCACGCCTGCGCACGCTCCCAC
CGGCCGGAGACAGCCAGGAGCGCTCGGACAGCCCCGAGATCTGCCATTAC
GAGAAGAGCTTTCACAAGCACTCGGCCACCCCCAACTACACGCACTGTGG
CCTCTTCGGGGACCCACACCTCAGGACTTTCACCGACCGCTTCCAGACCT
GCAAGGTGCAGGGCGCCTGGCCGCTCATCGACAATAATTACCTGAACGTG
CAGGTCACCAACACGCCTGTGCTGCCCGGCTCAGCGGCCACTGCCACCAG
CAAGCTCACCATCATCTTCAAGAACTTCCAGGAGTGTGTGGACCAGAAGG
TGTACCAGGCTGAGATGGACGAGCTCCCGGCCGCCTTCGTGGATGGCTCT
AAGAACGGTGGGGACAAGCACGGGGCCAACAGCCTGAAGATCACTGAGAA
GGTGTCAGGCCAGCACGTGGAGATCCAGGCCAAGTACATCGGCACCACCA
TCGTGGTGCGCCAGGTGGGCCGCTACCTGACCTTTGCCGTCCGCATGCCA
GAGGAAGTGGTCAATGCTGTGGAGGACTGGGACAGCCAGGGTCTCTACCT
CTGCCTGCGGGGCTGCCCCCTCAACCAGCAGATCGACTTCCAGGCCTTCC
ACACCAATGCTGAGGGCACCGGTGCCCGCAGGCTGGCAGCCGCCAGCCCT
GCACCCACAGCCCCCGAGACCTTCCCATACGAGACAGCCGTGGCCAAGTG
CAAGGAGAAGCTGCCGGTGGAGGACCTGTACTACCAGGCCTGCGTCTTCG
ACCTCCTCACCACGGGCGACGTGAACTTCACACTGGCCGCCTACTACGCG
TTGGAGGATGTCAAGATGCTCCACTCC

In certain embodiments, the disclosure relates to heteromultimers that comprise at least one RGM-A polypeptide, which includes fragments, functional variants, and modified forms thereof. Preferably, RGM-A polypeptides for use in accordance with the disclosure (e.g., heteromultimers comprising a RGM-A polypeptide and uses thereof) are soluble (e.g., an extracellular domain of RGM-A). In other preferred embodiments, RGM-A polypeptides for use in accordance with the disclosure bind to and/or inhibit (antagonize) activity (e.g., Smad signaling) of one or more TGF-beta superfamily ligands. In some embodiments, heteromultimers of the disclosure comprise at least one RGM-A polypeptide that is at least 70%, 75%, 80%, 85%, 90%, 91%, 92%, 93%, 94%, 95%, 96%, 97%, 98%, 99%, or 100% identical to the amino acid sequence of SEQ ID NOs: 561, 562, 565, 566, 569, or 570. In some embodiments, heteromultimers of the disclosure comprise at least one RGM-A polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 1-177 (e.g., amino acid residues 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, or 177) of SEQ ID NO: 561, and ends at any one of amino acids 430-458 (e.g., amino acid residues 430, 431, 432, 433, 434, 435, 435, 436, 437, 438, 439, 440, 441, 442, 443, 444, 445, 446, 447, 448, 449, 450, 451, 452, 453, 454, 455, 456, 457, or 458) of SEQ ID NO: 561. In some embodiments, heteromultimers of the disclosure comprise of at least one RGM-A polypeptide that is at least 70%, 75%, 80%, 85%, 90%, 91%, 92%, 93%, 94%, 95%, 96%, 97%, 98%, 99%, or 100% identical to amino acids of 1-430 of SEQ ID NO: 561. In some embodiments, heteromultimers of the disclosure comprise of at least one RGM-A polypeptide that is at least 70%, 75%, 80%, 85%, 90%, 91%, 92%, 93%, 94%, 95%, 96%, 97%, 98%, 99%, or 100% identical to amino acids of 1-458 of SEQ ID NO: 561. In some embodiments, heteromultimers of the disclosure comprise of at least one RGM-A polypeptide that is at least 70%, 75%, 80%, 85%, 90%, 91%, 92%, 93%, 94%, 95%, 96%, 97%, 98%, 99%, or 100% identical to amino acids of 177-430 of SEQ ID NO: 561. In some embodiments, heteromultimers of the disclosure comprise of at least one RGM-A polypeptide that is at least 70%, 75%, 80%, 85%, 90%, 91%, 92%, 93%, 94%, 95%, 96%, 97%, 98%, 99%, or 100% identical to amino acids of 177-458 of SEQ ID NO: 561. In some embodiments, heteromultimers of the disclosure comprise of at least one RGM-A polypeptide that is at least 70%, 75%, 80%, 85%, 90%, 91%, 92%, 93%, 94%, 95%, 96%, 97%, 98%, 99%, or 100% identical to amino acids of 56-430 of SEQ ID NO: 561. In some embodiments, heteromultimers of the disclosure comprise of at least one RGM-A polypeptide that is at least 70%, 75%, 80%, 85%, 90%, 91%, 92%, 93%, 94%, 95%, 96%, 97%, 98%, 99%, or 100% identical to amino acids of 56-458 of SEQ ID NO: 561. In some embodiments, heteromultimers of the disclosure comprise at least one RGM-A polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 1-153 (e.g., amino acid residues 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 135, 136, 137, 138, 139, 140, 141, 142, 143, 144, 145, 146, 147, 148, 149, 150, 151, 152, or 153) of SEQ ID NO: 565, and ends at any one of amino acids 406-434 (e.g., amino acid residues 406, 407, 408, 409, 410, 411, 412, 413, 414, 415, 416, 417, 418, 419, 420, 421, 422, 423, 424, 425, 426, 427, 428, 429, 430, 431, 432, 433, 434) of SEQ ID NO: 565. In some embodiments, heteromultimers of the disclosure comprise of at least one RGM-A polypeptide that is at least 70%, 75%, 80%, 85%, 90%, 91%, 92%, 93%, 94%, 95%, 96%, 97%, 98%, 99%, or 100% identical to amino acids of 1-406 of SEQ ID NO: 565. In some embodiments, heteromultimers of the disclosure comprise of at least one RGM-A polypeptide that is at least 70%, 75%, 80%, 85%, 90%, 91%, 92%, 93%, 94%, 95%, 96%, 97%, 98%, 99%, or 100% identical to amino acids of 153-406 of SEQ ID NO: 565. In some embodiments, heteromultimers of the disclosure comprise of at least one RGM-A polypeptide that is at least 70%, 75%, 80%, 85%, 90%, 91%, 92%, 93%, 94%, 95%, 96%, 97%, 98%, 99%, or 100% identical to amino acids of 1-434 of SEQ ID NO: 565. In some embodiments, heteromultimers of the disclosure comprise of at least one RGM-A polypeptide that is at least 70%, 75%, 80%, 85%, 90%, 91%, 92%, 93%, 94%, 95%, 96%, 97%, 98%, 99%, or 100% identical to amino acids of 153-434 of SEQ ID NO: 565. In some embodiments, heteromultimers of the disclosure comprise of at least one RGM-A polypeptide that is at least 70%, 75%, 80%, 85%, 90%, 91%, 92%, 93%, 94%, 95%, 96%, 97%, 98%, 99%, or 100% identical to amino acids of 32-406 of SEQ ID NO: 565. In some embodiments, heteromultimers of the disclosure comprise of at least one RGM-A polypeptide that is at least 70%, 75%, 80%, 85%, 90%, 91%, 92%, 93%, 94%, 95%, 96%, 97%, 98%, 99%, or 100% identical to amino acids of 32-434 of SEQ ID NO: 565. In some embodiments, heteromultimers of the disclosure comprise at least one RGM-A polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 1-169 (e.g., amino acid residues 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169) of SEQ ID NO: 569, and ends at any one of amino acids 422-450 (e.g., amino acid residues 422, 423, 424, 425, 426, 427, 428, 429, 430, 431, 432, 433, 434, 435, 435, 436, 437, 438, 439, 440, 441, 442, 443, 444, 445, 446, 447, 448, 449, 450) of SEQ ID NO: 569. In some embodiments, heteromultimers of the disclosure comprise of at least one RGM-A polypeptide that is at least 70%, 75%, 80%, 85%, 90%, 91%, 92%, 93%, 94%, 95%, 96%, 97%, 98%, 99%, or 100% identical to amino acids of 1-422 of SEQ ID NO: 569. In some embodiments, heteromultimers of the disclosure comprise of at least one RGM-A polypeptide that is at least 70%, 75%, 80%, 85%, 90%, 91%, 92%, 93%, 94%, 95%, 96%, 97%, 98%, 99%, or 100% identical to amino acids of 169-422 of SEQ ID NO: 569. In some embodiments, heteromultimers of the disclosure comprise of at least one RGM-A polypeptide that is at least 70%, 75%, 80%, 85%, 90%, 91%, 92%, 93%, 94%, 95%, 96%, 97%, 98%, 99%, or 100% identical to amino acids of 1-450 of SEQ ID NO: 569. In some embodiments, heteromultimers of the disclosure comprise of at least one RGM-A polypeptide that is at least 70%, 75%, 80%, 85%, 90%, 91%, 92%, 93%, 94%, 95%, 96%, 97%, 98%, 99%, or 100% identical to amino acids of 169-450 of SEQ ID NO: 569. In some embodiments, heteromultimers of the disclosure comprise of at least one RGM-A polypeptide that is at least 70%, 75%, 80%, 85%, 90%, 91%, 92%, 93%, 94%, 95%, 96%, 97%, 98%, 99%, or 100% identical to amino acids of 48-422 of SEQ ID NO: 569. In some embodiments, heteromultimers of the disclosure comprise of at least one RGM-A polypeptide that is at least 70%, 75%, 80%, 85%, 90%, 91%, 92%, 93%, 94%, 95%, 96%, 97%, 98%, 99%, or 100% identical to amino acids of 48-450 of SEQ ID NO: 569. In some embodiments, heteromultimers of the disclosure comprise at least one RGM-A polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 56-61 (e.g., amino acid residues 56, 57, 58, 59, 60, or 61) of SEQ ID NO: 561, and ends at any one of amino acids 366-458 (e.g., amino acid residues 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, or 458) of SEQ ID NO: 561. In some embodiments, heteromultimers of the disclosure comprise of at least one RGM-A polypeptide that is at least 70%, 75%, 80%, 85%, 90%, 91%, 92%, 93%, 94%, 95%, 96%, 97%, 98%, 99%, or 100% identical to amino acids of 56-458 of SEQ ID NO: 561. In some embodiments, heteromultimers of the disclosure comprise of at least one RGM-A polypeptide that is at least 70%, 75%, 80%, 85%, 90%, 91%, 92%, 93%, 94%, 95%, 96%, 97%, 98%, 99%, or 100% identical to amino acids of 61-366 of SEQ ID NO: 561. In some embodiments, heteromultimers of the disclosure comprise at least one RGM-A polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 32-37 (e.g., amino acid residues 32, 33, 34, 35, 36, or 37) of SEQ ID NO: 565, and ends at any one of amino acids 362-434 (e.g., amino acid residues 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, or 434) of SEQ ID NO: 565. In some embodiments, heteromultimers of the disclosure comprise of at least one RGM-A polypeptide that is at least 70%, 75%, 80%, 85%, 90%, 91%, 92%, 93%, 94%, 95%, 96%, 97%, 98%, 99%, or 100% identical to amino acids of 32-434 of SEQ ID NO: 565. In some embodiments, heteromultimers of the disclosure comprise of at least one RGM-A polypeptide that is at least 70%, 75%, 80%, 85%, 90%, 91%, 92%, 93%, 94%, 95%, 96%, 97%, 98%, 99%, or 100% identical to amino acids of 37-362 of SEQ ID NO: 565. In some embodiments, heteromultimers of the disclosure comprise at least one RGM-A polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 48-53 (e.g., amino acid residues 48, 49, 50, 51, 52, or 53) of SEQ ID NO: 569, and ends at any one of amino acids 378-450 (e.g., amino acid residues 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450) of SEQ ID NO: 569. In some embodiments, heteromultimers of the disclosure comprise of at least one RGM-A polypeptide that is at least 70%, 75%, 80%, 85%, 90%, 91%, 92%, 93%, 94%, 95%, 96%, 97%, 98%, 99%, or 100% identical to amino acids of 48-450 of SEQ ID NO: 569. In some embodiments, heteromultimers of the disclosure comprise of at least one RGM-A polypeptide that is at least 70%, 75%, 80%, 85%, 90%, 91%, 92%, 93%, 94%, 95%, 96%, 97%, 98%, 99%, or 100% identical to amino acids of 53-378 of SEQ ID NO: 569.

The term “hemojuvelin polypeptide” includes polypeptides comprising any naturally occurring hemojuvelin protein (encoded by HFE2 or one of its nonhuman orthologs) as well as any variants thereof (including mutants, fragments, fusions, and peptidomimetic forms) that retain a useful activity.

The human hemojuvelin isoform A precursor protein sequence (NCBI Ref Seq NP_998818.1) is as follows:

| (SEQ ID NO: 573) | |
|---|---|
| 1 | MGEPGQSPSP RSSHGSPPTL STLTLLLLLC GHAHSQCKIL RCNAEYVSST LSLRGGGSSG |
| 61 | ALRGGGGGGR GGGVGSGGLC RALRSYALCT RRTARTCRGD LAFHSAVHGI EDLMIQHNCS |
| 121 | RQGPTAPPPP RGPALPGAGS GLPAPDPCDY EGRFSRLHGR PPGFLHCASF GDPHVRSFHH |
| 181 | HFHTCRVQGA WPLLDNDFLF VQATSSPMAL GANATATRKL TIIFKNMQEC IDQKVYQAEV |
| 241 | DNLPVAFEDG SINGGDRPGG SSLSIQTANP GNHVEIQAAY IGTTIIIRQT AGQLSFSIKV |
| 301 | AEDVAMAFSA EQDLQLCVGG CPPSQRLSRS ERNRRGAITI DTARRLCKEG LPVEDAYFHS |
| 361 | CVFDVLISGD PNFTVAAQAA LEDARAFLPD LEKLHLFPSD AGVPLSSATL LAPLLSGLFV |
| 421 | LWLCIQ |

The signal peptide is indicated by single underline.

A processed hemojuvelin isoform A polypeptide sequence is as follows:

(SEQ ID NO: 574)

QCKILRCNAEYVSSTLSLRGGGSSGALRGGGGGGRGGGVGSGGLCRALRS
YALCTRRTARTCRGDLAFHSAVHGIEDLMIQHNCSRQGPTAPPPPRGPAL
PGAGSGLPAPDPCDYEGRFSRLHGRPPGFLHCASFGDPHVRSFHHHFHTC
RVQGAWPLLDNDFLFVQATSSPMALGANATATRKLTIIFKNMQECIDQKV
YQAEVDNLPVAFEDGSINGGDRPGGSSLSIQTANPGNHVEIQAAYIGTTI
IIRQTAGQLSFSIKVAEDVAMAFSAEQDLQLCVGGCPPSQRLSRSERNRR
GAITIDTARRLCKEGLPVEDAYFHSCVFDVLISGDPNFTVAAQAALEDAR
AFLPDLEKLHLFPSD

A nucleic acid sequence encoding unprocessed human hemojuvelin isoform A precursor protein is shown below (SEQ ID NO: 575), corresponding to nucleotides 326-1603 of NCBI Reference Sequence NM_213653.3. The signal sequence is underlined.

(SEQ ID NO: 575)

ATGGGGGAGCCAGGCCAGTCCCCTAGTCCCAGGTCCTCCCATGGCAGTCC
CCCAACTCTAAGCACTCTCACTCTCCTGCTGCTCCTCTGTGGACATGCTC
ATTCTCAATGCAAGATCCTCCGCTGCAATGCTGAGTACGTATCGTCCACT
CTGAGCCTTAGAGGTGGGGGTTCATCAGGAGCACTTCGAGGAGGAGGAGG
AGGAGGCCGGGGTGGAGGGGTGGGCTCTGGCGGCCTCTGTCGAGCCCTCC
GCTCCTATGCGCTCTGCACTCGGCGCACCGCCCGCACCTGCCGCGGGGAC
CTCGCCTTCCATTCGGCGGTACATGGCATCGAAGACCTGATGATCCAGCA
CAACTGCTCCCGCCAGGGCCCTACAGCCCCTCCCCCGCCCCGGGGCCCCG
CCCTTCCAGGCGCGGGCTCCGGCCTCCCTGCCCCGGACCCTTGTGACTAT
GAAGGCCGGTTTTCCCGGCTGCATGGTCGTCCCCCGGGGTTCTTGCATTG
CGCTTCCTTCGGGGACCCCCATGTGCGCAGCTTCCACCATCACTTTCACA
CATGCCGTGTCCAAGGAGCTTGGCCTCTACTGGATAATGACTTCCTCTTT
GTCCAAGCCACCAGCTCCCCCATGGCGTTGGGGGCCAACGCTACCGCCAC
CCGGAAGCTCACCATCATATTTAAGAACATGCAGGAATGCATTGATCAGA
AGGTGTATCAGGCTGAGGTGGATAATCTTCCTGTAGCCTTTGAAGATGGT
TCTATCAATGGAGGTGACCGACCTGGGGGATCCAGTTTGTCGATTCAAAC
TGCTAACCCTGGGAACCATGTGGAGATCCAAGCTGCCTACATTGGCACAA
CTATAATCATTCGGCAGACAGCTGGGCAGCTCTCCTTCTCCATCAAGGTA
GCAGAGGATGTGGCCATGGCCTTCTCAGCTGAACAGGACCTGCAGCTCTG
TGTTGGGGGGTGCCCTCCAAGTCAGCGACTCTCTCGATCAGAGCGCAATC
GTCGGGGAGCTATAACCATTGATACTGCCAGACGGCTGTGCAAGGAAGGG
CTTCCAGTGGAAGATGCTTACTTCCATTCCTGTGTCTTTGATGTTTTAAT
TTCTGGTGATCCCAACTTTACCGTGGCAGCTCAGGCAGCACTGGAGGATG
CCCGAGCCTTCCTGCCAGACTTAGAGAAGCTGCATCTCTTCCCCTCAGAT
GCTGGGGTTCCTCTTTCCTCAGCAACCCTCTTAGCTCCACTCCTTTCTGG
GCTCTTTGTTCTGTGGCTTTGCATTCAG

A nucleic acid sequence encoding a processed hemojuvelin isoform A is shown below (SEQ ID NO: 576):

(SEQ ID NO: 576)

CAATGCAAGATCCTCCGCTGCAATGCTGAGTACGTATCGTCCACTCTGAG
CCTTAGAGGTGGGGGTTCATCAGGAGCACTTCGAGGAGGAGGAGGAGGAG
GCCGGGGTGGAGGGGTGGGCTCTGGCGGCCTCTGTCGAGCCCTCCGCTCC
TATGCGCTCTGCACTCGGCGCACCGCCCGCACCTGCCGCGGGGACCTCGC
CTTCCATTCGGCGGTACATGGCATCGAAGACCTGATGATCCAGCACAACT
GCTCCCGCCAGGGCCCTACAGCCCCTCCCCCGCCCCGGGGCCCCGCCCTT
CCAGGCGCGGGCTCCGGCCTCCCTGCCCCGGACCCTTGTGACTATGAAGG
CCGGTTTTCCCGGCTGCATGGTCGTCCCCCGGGGTTCTTGCATTGCGCTT
CCTTCGGGGACCCCCATGTGCGCAGCTTCCACCATCACTTTCACACATGC
CGTGTCCAAGGAGCTTGGCCTCTACTGGATAATGACTTCCTCTTTGTCCA
AGCCACCAGCTCCCCCATGGCGTTGGGGGCCAACGCTACCGCCACCCGGA
AGCTCACCATCATATTTAAGAACATGCAGGAATGCATTGATCAGAAGGTG
TATCAGGCTGAGGTGGATAATCTTCCTGTAGCCTTTGAAGATGGTTCTAT
CAATGGAGGTGACCGACCTGGGGGATCCAGTTTGTCGATTCAAACTGCTA
ACCCTGGGAACCATGTGGAGATCCAAGCTGCCTACATTGGCACAACTATA
ATCATTCGGCAGACAGCTGGGCAGCTCTCCTTCTCCATCAAGGTAGCAGA
GGATGTGGCCATGGCCTTCTCAGCTGAACAGGACCTGCAGCTCTGTGTTG
GGGGGTGCCCTCCAAGTCAGCGACTCTCTCGATCAGAGCGCAATCGTCGG
GGAGCTATAACCATTGATACTGCCAGACGGCTGTGCAAGGAAGGGCTTCC
AGTGGAAGATGCTTACTTCCATTCCTGTGTCTTTGATGTTTTAATTTCTG
GTGATCCCAACTTTACCGTGGCAGCTCAGGCAGCACTGGAGGATGCCCGA
GCCTTCCTGCCAGACTTAGAGAAGCTGCATCTCTTCCCCTCAGAT

A human hemojuvelin isoform B protein sequence (NCBI Ref Seq NP_660320.3) is as follows:

| (SEQ ID NO: 577) | |
|---|---|
| 1 | MIQHNCSRQG PTAPPPPRGP ALPGAGSGLP APDPCDYEGR FSRLHGRPPG FLHCASFGDP |
| 61 | HVRSFHHHFH TCRVQGAWPL LDNDFLFVQA TSSPMALGAN ATATRKLTII FKNMQECIDQ |
| 121 | KVYQAEVDNL PVAFEDGSIN GGDRPGGSSL SIQTANPGNH VEIQAAYIGT TIIIRQTAGQ |
| 181 | LSFSIKVAED VAMAFSAEQD LQLCVGGCPP SQRLSRSERN RRGAITIDTA RRLCKEGLPV |
| 241 | EDAYFHSCVF DVLISGDPNF TVAAQAALED ARAFLPDLEK LHLFPSDAGV PLSSATLLAP |
| 301 | LLSGLFVLWL CIQ |

A processed hemojuvelin isoform B polypeptide sequence is as follows:

(SEQ ID NO: 578)

MIQHNCSRQGPTAPPPPRGPALPGAGSGLPAPDPCDYEGRFSRLHGRPPG
FLHCASFGDPHVRSFHHHFHTCRVQGAWPLLDNDFLFVQATSSPMALGAN
ATATRKLTIIFKNMQECIDQKVYQAEVDNLPVAFEDGSINGGDRPGGSSL
SIQTANPGNHVEIQAAYIGTTIIIRQTAGQLSFSIKVAEDVAMAFSAEQD
LQLCVGGCPPSQRLSRSERNRRGAITIDTARRLCKEGLPVEDAYFHSCVF
DVLISGDPNFTVAAQAALEDARAFLPDLEKLHLFPSD

A nucleic acid sequence encoding human hemojuvelin isoform B precursor protein is shown below (SEQ ID NO: 579), corresponding to nucleotides 479-1417 of NCBI Reference Sequence NM_145277.4.

(SEQ ID NO: 579)

ATGATCCAGCACAACTGCTCCCGCCAGGGCCCTACAGCCCCTCCCCCGCC
CCGGGGCCCCGCCCTTCCAGGCGCGGGCTCCGGCCTCCCTGCCCCGGACC
CTTGTGACTATGAAGGCCGGTTTTCCCGGCTGCATGGTCGTCCCCCGGGG
TTCTTGCATTGCGCTTCCTTCGGGGACCCCCATGTGCGCAGCTTCCACCA
TCACTTTCACACATGCCGTGTCCAAGGAGCTTGGCCTCTACTGGATAATG
ACTTCCTCTTTGTCCAAGCCACCAGCTCCCCCATGGCGTTGGGGGCCAAC
GCTACCGCCACCCGGAAGCTCACCATCATATTTAAGAACATGCAGGAATG
CATTGATCAGAAGGTGTATCAGGCTGAGGTGGATAATCTTCCTGTAGCCT
TTGAAGATGGTTCTATCAATGGAGGTGACCGACCTGGGGGATCCAGTTTG
TCGATTCAAACTGCTAACCCTGGGAACCATGTGGAGATCCAAGCTGCCTA
CATTGGCACAACTATAATCATTCGGCAGACAGCTGGGCAGCTCTCCTTCT
CCATCAAGGTAGCAGAGGATGTGGCCATGGCCTTCTCAGCTGAACAGGAC
CTGCAGCTCTGTGTTGGGGGGTGCCCTCCAAGTCAGCGACTCTCTCGATC
AGAGCGCAATCGTCGGGGAGCTATAACCATTGATACTGCCAGACGGCTGT
GCAAGGAAGGGCTTCCAGTGGAAGATGCTTACTTCCATTCCTGTGTCTTT
GATGTTTTAATTTCTGGTGATCCCAACTTTACCGTGGCAGCTCAGGCAGC
ACTGGAGGATGCCCGAGCCTTCCTGCCAGACTTAGAGAAGCTGCATCTCT
TCCCCTCAGATGCTGGGGTTCCTCTTTCCTCAGCAACCCTCTTAGCTCCA
CTCCTTTCTGGGCTCTTTGTTCTGTGGCTTTGCATTCAG

A nucleic acid sequence encoding a processed hemojuvelin isoform B is shown below (SEQ ID NO: 580):

(SEQ ID NO: 580)

ATGATCCAGCACAACTGCTCCCGCCAGGGCCCTACAGCCCCTCCCCCGCC
CCGGGGCCCCGCCCTTCCAGGCGCGGGCTCCGGCCTCCCTGCCCCGGACC
CTTGTGACTATGAAGGCCGGTTTTCCCGGCTGCATGGTCGTCCCCCGGGG
TTCTTGCATTGCGCTTCCTTCGGGGACCCCCATGTGCGCAGCTTCCACCA
TCACTTTCACACATGCCGTGTCCAAGGAGCTTGGCCTCTACTGGATAATG
ACTTCCTCTTTGTCCAAGCCACCAGCTCCCCCATGGCGTTGGGGGCCAAC
GCTACCGCCACCCGGAAGCTCACCATCATATTTAAGAACATGCAGGAATG
CATTGATCAGAAGGTGTATCAGGCTGAGGTGGATAATCTTCCTGTAGCCT
TTGAAGATGGTTCTATCAATGGAGGTGACCGACCTGGGGGATCCAGTTTG
TCGATTCAAACTGCTAACCCTGGGAACCATGTGGAGATCCAAGCTGCCTA
CATTGGCACAACTATAATCATTCGGCAGACAGCTGGGCAGCTCTCCTTCT
CCATCAAGGTAGCAGAGGATGTGGCCATGGCCTTCTCAGCTGAACAGGAC
CTGCAGCTCTGTGTTGGGGGGTGCCCTCCAAGTCAGCGACTCTCTCGATC
AGAGCGCAATCGTCGGGGAGCTATAACCATTGATACTGCCAGACGGCTGT
GCAAGGAAGGGCTTCCAGTGGAAGATGCTTACTTCCATTCCTGTGTCTTT
GATGTTTTAATTTCTGGTGATCCCAACTTTACCGTGGCAGCTCAGGCAGC
ACTGGAGGATGCCCGAGCCTTCCTGCCAGACTTAGAGAAGCTGCATCTCT
TCCCCTCAGAT

A human hemojuvelin isoform C protein sequence (NCBI Ref Seq NP_973733.1) is as follows:

| (SEQ ID NO: 581) | |
|---|---|
| 1 | MQECIDQKVY QAEVDNLPVA FEDGSINGGD RPGGSSLSIQ TANPGNHVEI QAAYIGTTII |
| 61 | IRQTAGQLSF SIKVAEDVAM AFSAEQDLQL CVGGCPPSQR LSRSERNRRG AITIDTARRL |
| 121 | CKEGLPVEDA YFHSCVFDVL ISGDPNFTVA AQAALEDARA FLPDLEKLHL FPSD |

A processed hemojuvelin isoform C polypeptide sequence is as follows:

(SEQ ID NO: 582)
MQECIDQKVYQAEVDNLPVAFEDGSINGGDRPGGSSLSIQTANPGNHVEI
QAAYIGTTIIIRQTAGQLSFSIKVAEDVAMAFSAEQDLQLCVGGCPPSQR
LSRSERNRRGAITIDTARRLCKEGLPVEDAYFHSCVFDVLISGDPNFTVA
AQAALEDARAFLPDLEKLHLFPSD

A nucleic acid sequence encoding human hemojuvelin isoform C protein is shown below (SEQ ID NO: 583), corresponding to nucleotides 295-894 of NCBI Reference Sequence NM_202004.3.

(SEQ ID NO: 583)
ATGCAGGAATGCATTGATCAGAAGGTGTATCAGGCTGAGGTGGATAATCT
TCCTGTAGCCTTTGAAGATGGTTCTATCAATGGAGGTGACCGACCTGGGG
GATCCAGTTTGTCGATTCAAACTGCTAACCCTGGGAACCATGTGGAGATC
CAAGCTGCCTACATTGGCACAACTATAATCATTCGGCAGACAGCTGGGCA
GCTCTCCTTCTCCATCAAGGTAGCAGAGGATGTGGCCATGGCCTTCTCAG
CTGAACAGGACCTGCAGCTCTGTGTTGGGGGGTGCCCTCCAAGTCAGCGA
CTCTCTCGATCAGAGCGCAATCGTCGGGGAGCTATAACCATTGATACTGC
CAGACGGCTGTGCAAGGAAGGGCTTCCAGTGGAAGATGCTTACTTCCATT
CCTGTGTCTTTGATGTTTTAATTTCTGGTGATCCCAACTTTACCGTGGCA
GCTCAGGCAGCACTGGAGGATGCCCGAGCCTTCCTGCCAGACTTAGAGAA
GCTGCATCTCTTCCCCTCAGATGCTGGGGTTCCTCTTTCCTCAGCAACCC
TCTTAGCTCCACTCCTTTCTGGGCTCTTTGTTCTGTGGCTTTGCATTCAG

A nucleic acid sequence encoding a processed hemojuvelin isoform C is shown below (SEQ ID NO: 584):

(SEQ ID NO: 584)
ATGCAGGAATGCATTGATCAGAAGGTGTATCAGGCTGAGGTGGATAATCT
TCCTGTAGCCTTTGAAGATGGTTCTATCAATGGAGGTGACCGACCTGGGG
GATCCAGTTTGTCGATTCAAACTGCTAACCCTGGGAACCATGTGGAGATC
CAAGCTGCCTACATTGGCACAACTATAATCATTCGGCAGACAGCTGGGCA
GCTCTCCTTCTCCATCAAGGTAGCAGAGGATGTGGCCATGGCCTTCTCAG
CTGAACAGGACCTGCAGCTCTGTGTTGGGGGGTGCCCTCCAAGTCAGCGA
CTCTCTCGATCAGAGCGCAATCGTCGGGGAGCTATAACCATTGATACTGC
CAGACGGCTGTGCAAGGAAGGGCTTCCAGTGGAAGATGCTTACTTCCATT
CCTGTGTCTTTGATGTTTTAATTTCTGGTGATCCCAACTTTACCGTGGCA
GCTCAGGCAGCACTGGAGGATGCCCGAGCCTTCCTGCCAGACTTAGAGAA
GCTGCATCTCTTCCCCTCAGAT

In certain embodiments, the disclosure relates to heteromultimers that comprise at least one hemojuvelin polypeptide, which includes fragments, functional variants, and modified forms thereof. Preferably, hemojuvelin polypeptides for use in accordance with the disclosure (e.g., heteromultimers comprising a hemojuvelin polypeptide and uses thereof) are soluble (e.g., an extracellular domain of hemojuvelin). In other preferred embodiments, hemojuvelin polypeptides for use in accordance with disclosure bind to and/or inhibit (antagonize) activity (e.g., Smad signaling) of one or more TGF-beta superfamily ligands. In some embodiments, heteromultimers of the disclosure comprise at least one hemojuvelin polypeptide that is at least 70%, 75%, 80%, 85%, 90%, 91%, 92%, 93%, 94%, 95%, 96%, 97%, 98%, 99%, or 100% identical to the amino acid sequence of SEQ ID NOs: 573, 574, 577, 578, 581, or 582. In some embodiments, heteromultimers of the disclosure comprise at least one hemojuvelin polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 1-36 (e.g., amino acid residues 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 35, or 36) of SEQ ID NO: 573, and ends at any one of amino acids 400-426 (e.g., amino acid residues 400, 401, 402, 403, 404, 405, 406, 407, 408, 409, 410, 411, 412, 413, 414, 415, 416, 417, 418, 419, 420, 421, 422, 423, 424, 425, or 426) of SEQ ID NO: 573. In some embodiments, heteromultimers of the disclosure comprise at least one hemojuvelin polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 36-42 (e.g., amino acid residues 36, 37, 38, 39, 40, 41, or 42) of SEQ ID NO: 573, and ends at any one of amino acids 167-172 (e.g., amino acid residues 167, 168, 169, 170, 171, or 172) of SEQ ID NO: 573. In some embodiments, heteromultimers of the disclosure comprise at least one hemojuvelin polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 173-185 (e.g., amino acid residues 173, 174, 175, 176, 177, 178, 179, 180, 181, 182, 183, 184, or 185) of SEQ ID NO: 573, and ends at any one of amino acids 361-400 (e.g., amino acid residues 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400) of SEQ ID NO: 573. In some embodiments, heteromultimers of the disclosure comprise of at least one hemojuvelin polypeptide that is at least 70%, 75%, 80%, 85%, 90%, 91%, 92%, 93%, 94%, 95%, 96%, 97%, 98%, 99%, or 100% identical to amino acids of 1-400 of SEQ ID NO: 573. In some embodiments, heteromultimers of the disclosure comprise of at least one hemojuvelin polypeptide that is at least 70%, 75%, 80%, 85%, 90%, 91%, 92%, 93%, 94%, 95%, 96%, 97%, 98%, 99%, or 100% identical to amino acids of 1-426 of SEQ ID NO: 573. In some embodiments, heteromultimers of the disclosure comprise of at least one hemojuvelin polypeptide that is at least 70%, 75%, 80%, 85%, 90%, 91%, 92%, 93%, 94%, 95%, 96%, 97%, 98%, 99%, or 100% identical to amino acids of 36-400 of SEQ ID NO: 573. In some embodiments, heteromultimers of the disclosure comprise of at least one hemojuvelin polypeptide that is at least 70%, 75%, 80%, 85%, 90%, 91%, 92%, 93%, 94%, 95%, 96%, 97%, 98%, 99%, or 100% identical to amino acids of 36-426 of SEQ ID NO: 573. In some embodiments, heteromultimers of the disclosure comprise of at least one hemojuvelin polypeptide that is at least 70%, 75%, 80%, 85%, 90%, 91%, 92%, 93%, 94%, 95%, 96%, 97%, 98%, 99%, or 100% identical to amino acids of 36-167 of SEQ ID NO: 573. In some embodiments, heteromultimers of the disclosure comprise of at least one hemojuvelin polypeptide that is at least 70%, 75%, 80%, 85%, 90%, 91%, 92%, 93%, 94%, 95%, 96%, 97%, 98%, 99%, or 100% identical to amino acids of 36-172 of SEQ ID NO: 573. In some embodiments, heteromultimers of the disclosure comprise of at least one hemojuvelin polypeptide that is at least 70%, 75%, 80%, 85%, 90%, 91%, 92%, 93%, 94%, 95%, 96%, 97%, 98%, 99%, or 100% identical to amino acids of 42-167 of SEQ ID NO: 573. In some embodiments, heteromultimers of the disclosure comprise of at least one hemojuvelin polypeptide that is at least 70%, 75%, 80%, 85%, 90%, 91%, 92%, 93%, 94%, 95%, 96%, 97%, 98%, 99%, or 100% identical to amino acids of 42-172 of SEQ ID NO: 573. In some embodiments, heteromultimers of the disclosure comprise of at least one hemojuvelin polypeptide that is at least 70%, 75%, 80%, 85%, 90%, 91%, 92%, 93%, 94%, 95%, 96%, 97%, 98%, 99%, or 100% identical to amino acids of 173-361 of SEQ ID NO: 573. In some embodiments, heteromultimers of the disclosure comprise of at least one hemojuvelin polypeptide that is at least 70%, 75%, 80%, 85%, 90%, 91%, 92%, 93%, 94%, 95%, 96%, 97%, 98%, 99%, or 100% identical to amino acids of 173-400 of SEQ ID NO: 573. In some embodiments, heteromultimers of the disclosure comprise of at least one hemojuvelin polypeptide that is at least 70%, 75%, 80%, 85%, 90%, 91%, 92%, 93%, 94%, 95%, 96%, 97%, 98%, 99%, or 100% identical to amino acids of 185-361 of SEQ ID NO: 573. In some embodiments, heteromultimers of the disclosure comprise of at least one hemojuvelin polypeptide that is at least 70%, 75%, 80%, 85%, 90%, 91%, 92%, 93%, 94%, 95%, 96%, 97%, 98%, 99%, or 100% identical to amino acids of 185-400 of SEQ ID NO: 573. In some embodiments, heteromultimers of the disclosure comprise at least one hemojuvelin protein, wherein the hemojuvelin protein is a dimer comprising a first polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 36-42 (e.g., amino acid residues 36, 37, 38, 39, 40, 41, or 42) of SEQ ID NO: 573, and ends at any one of amino acids 167-172 (e.g., amino acid residues 167, 168, 169, 170, 171, or 172) of SEQ ID NO: 573, and second polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 173-185 (e.g., amino acid residues 173, 174, 175, 176, 177, 178, 179, 180, 181, 182, 183, 184, or 185) of SEQ ID NO: 573, and ends at any one of amino acids 361-400 (e.g., amino acid residues 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400) of SEQ ID NO: 573. In some embodiments, heteromultimers of the disclosure comprise at least one single chain ligand trap that comprises a first hemojuvelin polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 36-42 (e.g., amino acid residues 36, 37, 38, 39, 40, 41, or 42) of SEQ ID NO: 573, and ends at any one of amino acids 167-172 (e.g., amino acid residues 167, 168, 169, 170, 171, or 172) of SEQ ID NO: 573, and second hemojuvelin polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 173-185 (e.g., amino acid residues 173, 174, 175, 176, 177, 178, 179, 180, 181, 182, 183, 184, or 185) of SEQ ID NO: 573, and ends at any one of amino acids 361-400 (e.g., amino acid residues 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400) of SEQ ID NO: 573. In some embodiments, heteromultimers of the disclosure comprise at least one hemojuvelin polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 1-6 (e.g., amino acid residues 1, 2, 3, 4, 5, or 6) of SEQ ID NO: 577, and ends at any one of amino acids 287-313 (e.g., amino acid residues 287, 288, 289, 300, 301, 302, 303, 304, 305, 306, 307, 308, 309, 310, 311, 312, or 313) of SEQ ID NO: 577. In some embodiments, heteromultimers of the disclosure comprise at least one hemojuvelin polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 1-6 (e.g., amino acid residues 1, 2, 3, 4, 5, or 6) of SEQ ID NO: 577, and ends at any one of amino acids 54-59 (e.g., amino acid residues 54, 55, 56, 57, 58, or 59) of SEQ ID NO: 577. In some embodiments, heteromultimers of the disclosure comprise at least one hemojuvelin polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 60-72 (e.g., amino acid residues 60, 61, 62, 63, 64, 65, 66, 67, 68, 69, 70, 71, or 72) of SEQ ID NO: 577, and ends at any one of amino acids 248-287 (e.g., amino acid residues 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, or 287) of SEQ ID NO: 577. In some embodiments, heteromultimers of the disclosure comprise of at least one hemojuvelin polypeptide that is at least 70%, 75%, 80%, 85%, 90%, 91%, 92%, 93%, 94%, 95%, 96%, 97%, 98%, 99%, or 100% identical to amino acids of 1-287 of SEQ ID NO: 577. In some embodiments, heteromultimers of the disclosure comprise of at least one hemojuvelin polypeptide that is at least 70%, 75%, 80%, 85%, 90%, 91%, 92%, 93%, 94%, 95%, 96%, 97%, 98%, 99%, or 100% identical to amino acids of 1-313 of SEQ ID NO: 577. In some embodiments, heteromultimers of the disclosure comprise of at least one hemojuvelin polypeptide that is at least 70%, 75%, 80%, 85%, 90%, 91%, 92%, 93%, 94%, 95%, 96%, 97%, 98%, 99%, or 100% identical to amino acids of 6-287 of SEQ ID NO: 577. In some embodiments, heteromultimers of the disclosure comprise of at least one hemojuvelin polypeptide that is at least 70%, 75%, 80%, 85%, 90%, 91%, 92%, 93%, 94%, 95%, 96%, 97%, 98%, 99%, or 100% identical to amino acids of 6-313 of SEQ ID NO: 577. In some embodiments, heteromultimers of the disclosure comprise of at least one hemojuvelin polypeptide that is at least 70%, 75%, 80%, 85%, 90%, 91%, 92%, 93%, 94%, 95%, 96%, 97%, 98%, 99%, or 100% identical to amino acids of 1-54 of SEQ ID NO: 577. In some embodiments, heteromultimers of the disclosure comprise of at least one hemojuvelin polypeptide that is at least 70%, 75%, 80%, 85%, 90%, 91%, 92%, 93%, 94%, 95%, 96%, 97%, 98%, 99%, or 100% identical to amino acids of 1-59 of SEQ ID NO: 577. In some embodiments, heteromultimers of the disclosure comprise of at least one hemojuvelin polypeptide that is at least 70%, 75%, 80%, 85%, 90%, 91%, 92%, 93%, 94%, 95%, 96%, 97%, 98%, 99%, or 100% identical to amino acids of 6-54 of SEQ ID NO: 577. In some embodiments, heteromultimers of the disclosure comprise of at least one hemojuvelin polypeptide that is at least 70%, 75%, 80%, 85%, 90%, 91%, 92%, 93%, 94%, 95%, 96%, 97%, 98%, 99%, or 100% identical to amino acids of 6-59 of SEQ ID NO: 577. In some embodiments, heteromultimers of the disclosure comprise of at least one hemojuvelin polypeptide that is at least 70%, 75%, 80%, 85%, 90%, 91%, 92%, 93%, 94%, 95%, 96%, 97%, 98%, 99%, or 100% identical to amino acids of 60-248 of SEQ ID NO: 577. In some embodiments, heteromultimers of the disclosure comprise of at least one hemojuvelin polypeptide that is at least 70%, 75%, 80%, 85%, 90%, 91%, 92%, 93%, 94%, 95%, 96%, 97%, 98%, 99%, or 100% identical to amino acids of 60-287 of SEQ ID NO: 577. In some embodiments, heteromultimers of the disclosure comprise of at least one hemojuvelin polypeptide that is at least 70%, 75%, 80%, 85%, 90%, 91%, 92%, 93%, 94%, 95%, 96%, 97%, 98%, 99%, or 100% identical to amino acids of 72-248 of SEQ ID NO: 577. In some embodiments, heteromultimers of the disclosure comprise of at least one hemojuvelin polypeptide that is at least 70%, 75%, 80%, 85%, 90%, 91%, 92%, 93%, 94%, 95%, 96%, 97%, 98%, 99%, or 100% identical to amino acids of 72-287 of SEQ ID NO: 577. In some embodiments, heteromultimers of the disclosure comprise at least one hemojuvelin protein, wherein the hemojuvelin protein is a dimer comprising a first polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 1-6 (e.g., amino acid residues 1, 2, 3, 4, 5, or 6) of SEQ ID NO: 577, and ends at any one of amino acids 54-59 (e.g., amino acid residues 54, 55, 56, 57, 58, or 59) of SEQ ID NO: 577, and second polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 60-72 (e.g., amino acid residues 60, 61, 62, 63, 64, 65, 66, 67, 68, 69, 70, 71, or 72) of SEQ ID NO: 577, and ends at any one of amino acids 248-287 (e.g., amino acid residues 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, or 287) of SEQ ID NO: 577. In some embodiments, heteromultimers of the disclosure comprise at least one single chain ligand trap that comprises a first hemojuvelin polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 1-6 (e.g., amino acid residues 1, 2, 3, 4, 5, or 6) of SEQ ID NO: 577, and ends at any one of amino acids 54-59 (e.g., amino acid residues 54, 55, 56, 57, 58, or 59) of SEQ ID NO: 577, and second hemojuvelin polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 60-72 (e.g., amino acid residues 60, 61, 62, 63, 64, 65, 66, 67, 68, 69, 70, 71, or 72) of SEQ ID NO: 577, and ends at any one of amino acids 248-287 (e.g., amino acid residues 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, or 287) of SEQ ID NO: 577. In some embodiments, heteromultimers of the disclosure comprise at least one hemojuvelin polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 1-4 (e.g., amino acid residues 1, 2, 3, or 4) of SEQ ID NO: 581, and ends at any one of amino acids 135-200 (e.g., amino acid residues 135, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200) of SEQ ID NO: 581. In some embodiments, heteromultimers of the disclosure comprise of at least one hemojuvelin polypeptide that is at least 70%, 75%, 80%, 85%, 90%, 91%, 92%, 93%, 94%, 95%, 96%, 97%, 98%, 99%, or 100% identical to amino acids of 1-135 of SEQ ID NO: 581. In some embodiments, heteromultimers of the disclosure comprise of at least one hemojuvelin polypeptide that is at least 70%, 75%, 80%, 85%, 90%, 91%, 92%, 93%, 94%, 95%, 96%, 97%, 98%, 99%, or 100% identical to amino acids of 1-200 of SEQ ID NO: 581. In some embodiments, heteromultimers of the disclosure comprise of at least one hemojuvelin polypeptide that is at least 70%, 75%, 80%, 85%, 90%, 91%, 92%, 93%, 94%, 95%, 96%, 97%, 98%, 99%, or 100% identical to amino acids of 4-135 of SEQ ID NO: 581. In some embodiments, heteromultimers of the disclosure comprise of at least one hemojuvelin polypeptide that is at least 70%, 75%, 80%, 85%, 90%, 91%, 92%, 93%, 94%, 95%, 96%, 97%, 98%, 99%, or 100% identical to amino acids of 4-200 of SEQ ID NO: 581. In some embodiments, heteromultimers of the disclosure comprise of at least one hemojuvelin polypeptide that is at least 70%, 75%, 80%, 85%, 90%, 91%, 92%, 93%, 94%, 95%, 96%, 97%, 98%, 99%, or 100% identical to amino acids of 1-174 of SEQ ID NO: 581. In some embodiments, heteromultimers of the disclosure comprise of at least one hemojuvelin polypeptide that is at least 70%, 75%, 80%, 85%, 90%, 91%, 92%, 93%, 94%, 95%, 96%, 97%, 98%, 99%, or 100% identical to amino acids of 4-174 of SEQ ID NO: 581. In some embodiments, heteromultimers of the disclosure comprise at least one hemojuvelin polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 36-37 (e.g., amino acid residues 36 or 37) of SEQ ID NO: 573, and ends at any one of amino acids 424-426 (e.g., amino acid residues 424, 425, or 426) of SEQ ID NO: 573. In some embodiments, heteromultimers of the disclosure comprise of at least one hemojuvelin polypeptide that is at least 70%, 75%, 80%, 85%, 90%, 91%, 92%, 93%, 94%, 95%, 96%, 97%, 98%, 99%, or 100% identical to amino acids of 36-426 of SEQ ID NO: 573. In some embodiments, heteromultimers of the disclosure comprise of at least one hemojuvelin polypeptide that is at least 70%, 75%, 80%, 85%, 90%, 91%, 92%, 93%, 94%, 95%, 96%, 97%, 98%, 99%, or 100% identical to amino acids of 37-424 of SEQ ID NO: 573. In some embodiments, heteromultimers of the disclosure comprise of at least one hemojuvelin polypeptide that is at least 70%, 75%, 80%, 85%, 90%, 91%, 92%, 93%, 94%, 95%, 96%, 97%, 98%, 99%, or 100% identical to amino acids of 36-400 of SEQ ID NO: 573. In some embodiments, heteromultimers of the disclosure comprise at least one hemojuvelin polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 1-4 (e.g., amino acid residues 1, 2, 3, or 4) of SEQ ID NO: 582, and ends at any one of amino acids 135-174 (e.g., amino acid residues 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, or 174) of SEQ ID NO: 582. In some embodiments, heteromultimers of the disclosure comprise of at least one hemojuvelin polypeptide that is at least 70%, 75%, 80%, 85%, 90%, 91%, 92%, 93%, 94%, 95%, 96%, 97%, 98%, 99%, or 100% identical to amino acids of 1-174 of SEQ ID NO: 582. In some embodiments, heteromultimers of the disclosure comprise of at least one hemojuvelin polypeptide that is at least 70%, 75%, 80%, 85%, 90%, 91%, 92%, 93%, 94%, 95%, 96%, 97%, 98%, 99%, or 100% identical to amino acids of 4-135 of SEQ ID NO: 582. In some embodiments, heteromultimers of the disclosure comprise of at least one hemojuvelin polypeptide that is at least 70%, 75%, 80%, 85%, 90%, 91%, 92%, 93%, 94%, 95%, 96%, 97%, 98%, 99%, or 100% identical to amino acids of 1-174 of SEQ ID NO: 582. In some embodiments, heteromultimers of the disclosure comprise at least one hemojuvelin polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 1-6 (e.g., amino acid residues 1, 2, 3, 4, 5, or 6) of SEQ ID NO: 577, and ends at any one of amino acids 311-313 (e.g., amino acid residues 311, 312, or 313) of SEQ ID NO: 577. In some embodiments, heteromultimers of the disclosure comprise of at least one hemojuvelin polypeptide that is at least 70%, 75%, 80%, 85%, 90%, 91%, 92%, 93%, 94%, 95%, 96%, 97%, 98%, 99%, or 100% identical to amino acids of 1-313 of SEQ ID NO: 577. In some embodiments, heteromultimers of the disclosure comprise of at least one hemojuvelin polypeptide that is at least 70%, 75%, 80%, 85%, 90%, 91%, 92%, 93%, 94%, 95%, 96%, 97%, 98%, 99%, or 100% identical to amino acids of 6-311 of SEQ ID NO: 577. In some embodiments, heteromultimers of the disclosure comprise of at least one hemojuvelin polypeptide that is at least 70%, 75%, 80%, 85%, 90%, 91%, 92%, 93%, 94%, 95%, 96%, 97%, 98%, 99%, or 100% identical to amino acids of 1-127 of SEQ ID NO: 577.

The term “betaglycan polypeptide” includes polypeptides comprising any naturally occurring betaglycan protein (encoded by TGFBR3 or one of its nonhuman orthologs) as well as any variants thereof (including mutants, fragments, fusions, and peptidomimetic forms) that retain a useful activity.

The human betaglycan isoform A precursor protein sequence (NCBI Ref Seq NP_003234.2) is as follows:

```
(SEQ ID NO: 585)
    1 MTSHYVIAIF ALMSSCLATA GPEPGALCEL SPVSASHPVQ ALMESFTVLS GCASRGTTGL
  61 PQEVHVLNLR TAGQGPGQLQ REVTLHLNPI SSVHIHHKSV VFLLNSPHPL VWHLKTERLA
121 TGVSRLFLVS EGSVVQFSSA NFSLTAETEE RNFPHGNEHL LNWARKEYGA VTSFTELKIA
181 RNIYIKVGED QVFPPKCNIG KNFLSLNYLA EYLQPKAAEG CVMSSQPQNE EVHIIELITP
241 NSNPYSAFQV DITIDIRPSQ EDLEVVKNLI LILKCKKSVN WVIKSFDVKG SLKIIAPNSI

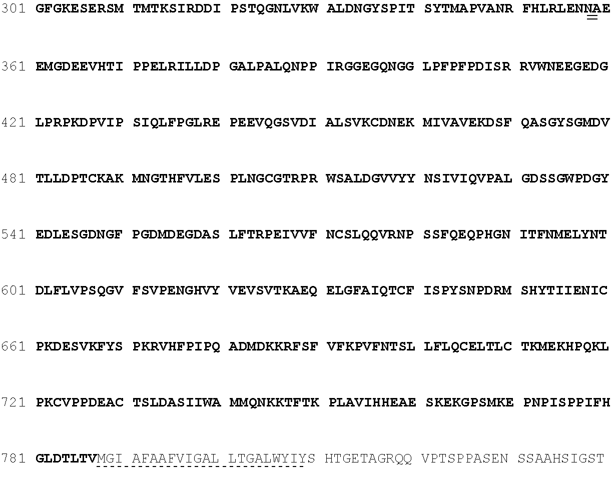

841 QSTPCSSSST A
```

The signal peptide is indicated by single underline, the extracellular domain is indicated in bold font, and the transmembrane domain is indicated by dotted underline. This isoform differs from betaglycan isoform B by insertion of a single alanine indicated above by double underline.

A processed betaglycan isoform A polypeptide sequence is as follows:

(SEQ ID NO: 586)
GPEPGALCELSPVSASHPVQALMESFTVLSGCASRGTTGLPQEVHVLNLR
TAGQGPGQLQREVTLHLNPISSVHIHHKSVVFLLNSPHPLVWHLKTERLA
TGVSRLFLVSEGSVVQFSSANFSLTAETEERNFPHGNEHLLNWARKEYGA
VTSFTELKIARNIYIKVGEDQVFPPKCNIGKNFLSLNYLAEYLQPKAAEG
CVMSSQPQNEEVHIIELITPNSNPYSAFQVDITIDIRPSQEDLEVVKNLI
LILKCKKSVNWVIKSFDVKGSLKIIAPNSIGFGKESERSMTMTKSIRDDI
PSTQGNLVKWALDNGYSPITSYTMAPVANRFHLRLENNAEEMGDEEVHTI
PPELRILLDPGALPALQNPPIRGGEGQNGGLPFPFPDISRRVWNEEGEDG
LPRPKDPVIPSIQLFPGLREPEEVQGSVDIALSVKCDNEKMIVAVEKDSF
QASGYSGMDVTLLDPTCKAKMNGTHFVLESPLNGCGTRPRWSALDGVVYY
NSIVIQVPALGDSSGWPDGYEDLESGDNGFPGDMDEGDASLFTRPEIVVF
NCSLQQVRNPSSFQEQPHGNITFNMELYNTDLFLVPSQGVFSVPENGHVY
VEVSVTKAEQELGFAIQTCFISPYSNPDRMSHYTIIENICPKDESVKFYS
PKRVHFPIPQADMDKKRFSFVFKPVFNTSLLFLQCELTLCTKMEKHPQKL
PKCVPPDEACTSLDASIIWAMMQNKKTFTKPLAVIHHEAESKEKGPSMKE
PNPISPPIFHGLDTLTV

A nucleic acid sequence encoding the unprocessed precursor protein of human betaglycan isoform A is shown below (SEQ ID NO: 587), corresponding to nucleotides 516-3068 of NCBI Reference Sequence NM_003243.4. The signal sequence is indicated by solid underline and the transmembrane region by dotted underline.

(SEQ ID NO: 587)
ATGACTTCCCATTATGTGATTGCCATCTTTGCCCTGATGAGCTCCTGTTTAGCCACTGCAGGTCCAGAGCCTGGT
GCACTGTGTGAACTGTCACCTGTCAGTGCCTCCCATCCTGTCCAGGCCTTGATGGAGAGCTTCACTGTTTTGTCA
GGCTGTGCCAGCAGAGGCACAACTGGGCTGCCACAGGAGGTGCATGTCCTGAATCTCCGCACTGCAGGCCAGGGG
CCTGGCCAGCTACAGAGAGAGGTCACACTTCACCTGAATCCCATCTCCTCAGTCCACATCCACCACAAGTCTGTT
GTGTTCCTGCTCAACTCCCCACACCCCCTGGTGTGGCATCTGAAGACAGAGAGACTTGCCACTGGGGTCTCCAGA
CTGTTTTTGGTGTCTGAGGGTTCTGTGGTCCAGTTTTCATCAGCAAACTTCTCCTTGACAGCAGAAACAGAAGAA
AGGAACTTCCCCCATGGAAATGAACATCTGTTAAATTGGGCCCGAAAAGAGTATGGAGCAGTTACTTCATTCACC
GAACTCAAGATAGCAAGAAACATTTATATTAAAGTGGGGGAAGATCAAGTGTTCCCTCCAAAGTGCAACATAGGG
AAGAATTTTCTCTCACTCAATTACCTTGCTGAGTACCTTCAACCCAAAGCAGCAGAAGGGTGTGTGATGTCCAGC
CAGCCCCAGAATGAGGAAGTACACATCATCGAGCTAATCACCCCCAACTCTAACCCCTACAGTGCTTTCCAGGTG
GATATAACAATTGATATAAGACCTTCTCAAGAGGATCTTGAAGTGGTCAAAAATCTCATCCTGATCTTGAAGTGC
AAAAAGTCTGTCAACTGGGTGATCAAATCTTTTGATGTTAAGGGAAGCCTGAAAATTATTGCTCCTAACAGTATT
GGCTTTGGAAAAGAGAGTGAAAGATCTATGACAATGACCAAATCAATAAGAGATGACATTCCTTCAACCCAAGGG
AATCTGGTGAAGTGGGCTTTGGACAATGGCTATAGTCCAATAACTTCATACACAATGGCTCCTGTGGCTAATAGA
TTTCATCTTCGGCTTGAAAATAATGCAGAGGAGATGGGAGATGAGGAAGTCCACACTATTCCTCCTGAGCTACGG
ATCCTGCTGGACCCTGGTGCCCTGCCTGCCCTGCAGAACCCGCCCATCCGGGGAGGGGAAGGCCAAAATGGAGGC
CTTCCGTTTCCTTTCCCAGATATTTCCAGGAGAGTCTGGAATGAAGAGGGAGAAGATGGGCTCCCTCGGCCAAAG
GACCCTGTCATTCCCAGCATACAACTGTTTCCTGGTCTCAGAGAGCCAGAAGAGGTGCAAGGGAGCGTGGATATT
GCCCTGTCTGTCAAATGTGACAATGAGAAGATGATCGTGGCTGTAGAAAAAGATTCTTTTCAGGCCAGTGGCTAC
TCGGGGATGGACGTCACCCTGTTGGATCCTACCTGCAAGGCCAAGATGAATGGCACACACTTTGTTTTGGAGTCT
CCTCTGAATGGCTGCGGTACTCGGCCCCGGTGGTCAGCCCTTGATGGTGTGGTCTACTATAACTCCATTGTGATA
CAGGTTCCAGCCCTTGGGGACAGTAGTGGTTGGCCAGATGGTTATGAAGATCTGGAGTCAGGTGATAATGGATTT
CCGGGAGATATGGATGAAGGAGATGCTTCCCTGTTCACCCGACCTGAAATCGTGGTGTTTAATTGCAGCCTTCAG
CAGGTGAGGAACCCCAGCAGCTTCCAGGAACAGCCCCACGGAAACATCACCTTCAACATGGAGCTATACAACACT
GACCTCTTTTTGGTGCCCTCCCAGGGCGTCTTCTCTGTGCCAGAGAATGGACACGTTTATGTTGAGGTATCTGTT
ACTAAGGCTGAACAAGAACTGGGATTTGCCATCCAAACGTGCTTTATCTCTCCATATTCGAACCCTGATAGGATG
TCTCATTACACCATTATTGAGAATATTTGTCCTAAAGATGAATCTGTGAAATTCTACAGTCCCAAGAGAGTGCAC
TTTCCTATCCCGCAAGCTGACATGGATAAGAAGCGATTCAGCTTTGTCTTCAAGCCTGTCTTCAACACCTCACTG
CTCTTTCTACAGTGTGAGCTGACGCTGTGTACGAAGATGGAGAAGCACCCCCAGAAGTTGCCTAAGTGTGTGCCT
CCTGACGAAGCCTGCACCTCGCTGGACGCCTCGATAATCTGGGCCATGATGCAGAATAAGAAGACGTTCACTAAG
CCCCTTGCTGTGATCCACCATGAAGCAGAATCTAAAGAAAAAGGTCCAAGCATGAAGGAACCAAATCCAATTTCT

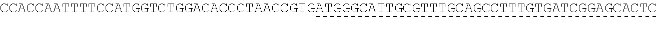

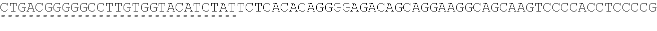

CCAGCCTCGGAAAACAGCAGTGCTGCCCACAGCATCGGCAGCACGCAGAGCACGCCTTGCTCCAGCAGCAGCACG
GCC

A nucleic acid sequence encoding a processed extracellular domain of betaglycan isoform A is shown below (SEQ ID NO: 588):

(SEQ ID NO: 588)
GGTCCAGAGCCTGGTGCACTGTGTGAACTGTCACCTGTCAGTGCCTCCCA
TCCTGTCCAGGCCTTGATGGAGAGCTTCACTGTTTTGTCAGGCTGTGCCA
GCAGAGGCACAACTGGGCTGCCACAGGAGGTGCATGTCCTGAATCTCCGC
ACTGCAGGCCAGGGGCCTGGCCAGCTACAGAGAGAGGTCACACTTCACCT
GAATCCCATCTCCTCAGTCCACATCCACCACAAGTCTGTTGTGTTCCTGC
TCAACTCCCCACACCCCCTGGTGTGGCATCTGAAGACAGAGAGACTTGCC
ACTGGGGTCTCCAGACTGTTTTTGGTGTCTGAGGGTTCTGTGGTCCAGTT
TTCATCAGCAAACTTCTCCTTGACAGCAGAAACAGAAGAAAGGAACTTCC
CCCATGGAAATGAACATCTGTTAAATTGGGCCCGAAAAGAGTATGGAGCA
GTTACTTCATTCACCGAACTCAAGATAGCAAGAAACATTTATATTAAAGT
GGGGGAAGATCAAGTGTTCCCTCCAAAGTGCAACATAGGGAAGAATTTTC
TCTCACTCAATTACCTTGCTGAGTACCTTCAACCCAAAGCAGCAGAAGGG
TGTGTGATGTCCAGCCAGCCCCAGAATGAGGAAGTACACATCATCGAGCT
AATCACCCCCAACTCTAACCCCTACAGTGCTTTCCAGGTGGATATAACAA
TTGATATAAGACCTTCTCAAGAGGATCTTGAAGTGGTCAAAAATCTCATC
CTGATCTTGAAGTGCAAAAAGTCTGTCAACTGGGTGATCAAATCTTTTGA
TGTTAAGGGAAGCCTGAAAATTATTGCTCCTAACAGTATTGGCTTTGGAA
AAGAGAGTGAAAGATCTATGACAATGACCAAATCAATAAGAGATGACATT
CCTTCAACCCAAGGGAATCTGGTGAAGTGGGCTTTGGACAATGGCTATAG
TCCAATAACTTCATACACAATGGCTCCTGTGGCTAATAGATTTCATCTTC
GGCTTGAAAATAATGCAGAGGAGATGGGAGATGAGGAAGTCCACACTATT
CCTCCTGAGCTACGGATCCTGCTGGACCCTGGTGCCCTGCCTGCCCTGCA
GAACCCGCCCATCCGGGGAGGGGAAGGCCAAAATGGAGGCCTTCCGTTTC
CTTTCCCAGATATTTCCAGGAGAGTCTGGAATGAAGAGGGAGAAGATGGG
CTCCCTCGGCCAAAGGACCCTGTCATTCCCAGCATACAACTGTTTCCTGG
TCTCAGAGAGCCAGAAGAGGTGCAAGGGAGCGTGGATATTGCCCTGTCTG
TCAAATGTGACAATGAGAAGATGATCGTGGCTGTAGAAAAAGATTCTTTT
CAGGCCAGTGGCTACTCGGGGATGGACGTCACCCTGTTGGATCCTACCTG
CAAGGCCAAGATGAATGGCACACACTTTGTTTTGGAGTCTCCTCTGAATG
GCTGCGGTACTCGGCCCCGGTGGTCAGCCCTTGATGGTGTGGTCTACTAT
AACTCCATTGTGATACAGGTTCCAGCCCTTGGGGACAGTAGTGGTTGGCC
AGATGGTTATGAAGATCTGGAGTCAGGTGATAATGGATTTCCGGGAGATA
TGGATGAAGGAGATGCTTCCCTGTTCACCCGACCTGAAATCGTGGTGTTT
AATTGCAGCCTTCAGCAGGTGAGGAACCCCAGCAGCTTCCAGGAACAGCC
CCACGGAAACATCACCTTCAACATGGAGCTATACAACACTGACCTCTTTT
TGGTGCCCTCCCAGGGCGTCTTCTCTGTGCCAGAGAATGGACACGTTTAT
GTTGAGGTATCTGTTACTAAGGCTGAACAAGAACTGGGATTTGCCATCCA
AACGTGCTTTATCTCTCCATATTCGAACCCTGATAGGATGTCTCATTACA
CCATTATTGAGAATATTTGTCCTAAAGATGAATCTGTGAAATTCTACAGT
CCCAAGAGAGTGCACTTTCCTATCCCGCAAGCTGACATGGATAAGAAGCG
ATTCAGCTTTGTCTTCAAGCCTGTCTTCAACACCTCACTGCTCTTTCTAC
AGTGTGAGCTGACGCTGTGTACGAAGATGGAGAAGCACCCCCAGAAGTTG
CCTAAGTGTGTGCCTCCTGACGAAGCCTGCACCTCGCTGGACGCCTCGAT
AATCTGGGCCATGATGCAGAATAAGAAGACGTTCACTAAGCCCCTTGCTG
TGATCCACCATGAAGCAGAATCTAAAGAAAAAGGTCCAAGCATGAAGGAA
CCAAATCCAATTTCTCCACCAATTTTCCATGGTCTGGACACCCTAACCGT
G

A human betaglycan isoform B precursor protein sequence (NCBI Ref Seq NP_001182612.1) is as follows:

| (SEQ ID NO: 589) | |
|---|---|
| 1 | MTSHYVIAIF ALMSSCLATA GPEPGALCEL SPVSASHPVQ ALMESFTVLS GCASRGTTGL |
| 61 | PQEVHVLNLR TAGQGPGQLQ REVTLHLNPI SSVHIHHKSV VFLLNSPHPL VWHLKTERLA |
| 121 | TGVSRLFLVS EGSVVQFSSA NFSLTAETEE RNFPHGNEHL LNWARKEYGA VTSFTELKIA |
| 181 | RNIYIKVGED QVFPPKCNIG KNFLSLNYLA EYLQPKAAEG CVMSSQPQNE EVHIIELITP |
| 241 | NSNPYSAFQV DITIDIRPSQ EDLEVVKNLI LILKCKKSVN WVIKSFDVKG SLKIIAPNSI |
| 301 | GFGKESERSM TMTKSIRDDI PSTQGNLVKW ALDNGYSPIT SYTMAPVANR FHLRLENNEE |
| 361 | MGDEEVHTIP PELRILLDPG ALPALQNPPI RGGEGQNGGL PFPFPDISRR VWNEEGEDGL |
| 421 | PRPKDPVIPS IQLFPGLREP EEVQGSVDIA LSVKCDNEKM IVAVEKDSFQ ASGYSGMDVT |
| 481 | LLDPTCKAKM NGTHFVLESP LNGCGTRPRW SALDGVVYYN SIVIQVPALG DSSGWPDGYE |
| 541 | DLESGDNGFP GDMDEGDASL FTRPEIVVFN CSLQQVRNPS SFQEQPHGNI TFNMELYNTD |
| 601 | LFLVPSQGVF SVPENGHVYV EVSVTKAEQE LGFAIQTCFI SPYSNPDRMS HYTIIENICP |
| 661 | KDESVKFYSP KRVHFPIPQA DMDKKRFSFV FKPVFNTSLL FLQCELTLCT KMEKHPQKLP |
| 721 | KCVPPDEACT SLDASIIWAM MQNKKTFTKP LAVIHHEAES KEKGPSMKEP NPISPPIFHG |
| 781 | 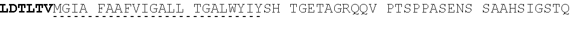 |
| 841 | STPCSSSSTA |

The signal peptide is indicated by single underline, the extracellular domain is indicated in bold font, and the transmembrane domain is indicated by dotted underline.

A processed betaglycan isoform B polypeptide sequence is as follows:

(SEQ ID NO: 590)
GPEPGALCELSPVSASHPVQALMESFTVLSGCASRGTTGLPQEVHVLNLR
TAGQGPGQLQREVTLHLNPISSVHIHHKSVVFLLNSPHPLVWHLKTERLA
TGVSRLFLVSEGSVVQFSSANFSLTAETEERNFPHGNEHLLNWARKEYGA
VTSFTELKIARNIYIKVGEDQVFPPKCNIGKNFLSLNYLAEYLQPKAAEG
CVMSSQPQNEEVHIIELITPNSNPYSAFQVDITIDIRPSQEDLEVVKNLI
LILKCKKSVNWVIKSFDVKGSLKIIAPNSIGFGKESERSMTMTKSIRDDI
PSTQGNLVKWALDNGYSPITSYTMAPVANRFHLRLENNEEMGDEEVHTIP
PELRILLDPGALPALQNPPIRGGEGQNGGLPFPFPDISRRVWNEEGEDGL
PRPKDPVIPSIQLFPGLREPEEVQGSVDIALSVKCDNEKMIVAVEKDSFQ
ASGYSGMDVTLLDPTCKAKMNGTHFVLESPLNGCGTRPRWSALDGVVYYN
SIVIQVPALGDSSGWPDGYEDLESGDNGFPGDMDEGDASLFTRPEIVVFN
CSLQQVRNPSSFQEQPHGNITFNMELYNTDLFLVPSQGVFSVPENGHVYV
EVSVTKAEQELGFAIQTCFISPYSNPDRMSHYTIIENICPKDESVKFYSP
KRVHFPIPQADMDKKRFSFVFKPVFNTSLLFLQCELTLCTKMEKHPQKLP
KCVPPDEACTSLDASIIWAMMQNKKTFTKPLAVIHHEAESKEKGPSMKEP
NPISPPIFHGLDTLTV

A nucleic acid sequence encoding the unprocessed precursor protein of human betaglycan isoform B is shown below (SEQ ID NO: 591), corresponding to nucleotides 516-3065 of NCBI Reference Sequence NM_001195683.1. The signal sequence is indicated by solid underline and the transmembrane region by dotted underline.

(SEQ ID NO: 591)
ATGACTTCCCATTATGTGATTGCCATCTTTGCCCTGATGAGCTCCTGTTTAGCCACTGCAGGTCCAGAGCCTGGT
GCACTGTGTGAACTGTCACCTGTCAGTGCCTCCCATCCTGTCCAGGCCTTGATGGAGAGCTTCACTGTTTTGTCA
GGCTGTGCCAGCAGAGGCACAACTGGGCTGCCACAGGAGGTGCATGTCCTGAATCTCCGCACTGCAGGCCAGGGG
CCTGGCCAGCTACAGAGAGAGGTCACACTTCACCTGAATCCCATCTCCTCAGTCCACATCCACCACAAGTCTGTT
GTGTTCCTGCTCAACTCCCCACACCCCCTGGTGTGGCATCTGAAGACAGAGAGACTTGCCACTGGGGTCTCCAGA
CTGTTTTTGGTGTCTGAGGGTTCTGTGGTCCAGTTTTCATCAGCAAACTTCTCCTTGACAGCAGAAACAGAAGAA
AGGAACTTCCCCCATGGAAATGAACATCTGTTAAATTGGGCCCGAAAAGAGTATGGAGCAGTTACTTCATTCACC
GAACTCAAGATAGCAAGAAACATTTATATTAAAGTGGGGGAAGATCAAGTGTTCCCTCCAAAGTGCAACATAGGG
AAGAATTTTCTCTCACTCAATTACCTTGCTGAGTACCTTCAACCCAAAGCAGCAGAAGGGTGTGTGATGTCCAGC
CAGCCCCAGAATGAGGAAGTACACATCATCGAGCTAATCACCCCCAACTCTAACCCCTACAGTGCTTTCCAGGTG
GATATAACAATTGATATAAGACCTTCTCAAGAGGATCTTGAAGTGGTCAAAAATCTCATCCTGATCTTGAAGTGC
AAAAAGTCTGTCAACTGGGTGATCAAATCTTTTGATGTTAAGGGAAGCCTGAAAATTATTGCTCCTAACAGTATT
GGCTTTGGAAAAGAGAGTGAAAGATCTATGACAATGACCAAATCAATAAGAGATGACATTCCTTCAACCCAAGGG
AATCTGGTGAAGTGGGCTTTGGACAATGGCTATAGTCCAATAACTTCATACACAATGGCTCCTGTGGCTAATAGA
TTTCATCTTCGGCTTGAAAATAATGAGGAGATGGGAGATGAGGAAGTCCACACTATTCCTCCTGAGCTACGGATC
CTGCTGGACCCTGGTGCCCTGCCTGCCCTGCAGAACCCGCCCATCCGGGGAGGGGAAGGCCAAAATGGAGGCCTT
CCGTTTCCTTTCCCAGATATTTCCAGGAGAGTCTGGAATGAAGAGGGAGAAGATGGGCTCCCTCGGCCAAAGGAC
CCTGTCATTCCCAGCATACAACTGTTTCCTGGTCTCAGAGAGCCAGAAGAGGTGCAAGGGAGCGTGGATATTGCC
CTGTCTGTCAAATGTGACAATGAGAAGATGATCGTGGCTGTAGAAAAAGATTCTTTTCAGGCCAGTGGCTACTCG
GGGATGGACGTCACCCTGTTGGATCCTACCTGCAAGGCCAAGATGAATGGCACACACTTTGTTTTGGAGTCTCCT
CTGAATGGCTGCGGTACTCGGCCCCGGTGGTCAGCCCTTGATGGTGTGGTCTACTATAACTCCATTGTGATACAG
GTTCCAGCCCTTGGGGACAGTAGTGGTTGGCCAGATGGTTATGAAGATCTGGAGTCAGGTGATAATGGATTTCCG
GGAGATATGGATGAAGGAGATGCTTCCCTGTTCACCCGACCTGAAATCGTGGTGTTTAATTGCAGCCTTCAGCAG
GTGAGGAACCCCAGCAGCTTCCAGGAACAGCCCCACGGAAACATCACCTTCAACATGGAGCTATACAACACTGAC
CTCTTTTTGGTGCCCTCCCAGGGCGTCTTCTCTGTGCCAGAGAATGGACACGTTTATGTTGAGGTATCTGTTACT
AAGGCTGAACAAGAACTGGGATTTGCCATCCAAACGTGCTTTATCTCTCCATATTCGAACCCTGATAGGATGTCT
CATTACACCATTATTGAGAATATTTGTCCTAAAGATGAATCTGTGAAATTCTACAGTCCCAAGAGAGTGCACTTT
CCTATCCCGCAAGCTGACATGGATAAGAAGCGATTCAGCTTTGTCTTCAAGCCTGTCTTCAACACCTCACTGCTC
TTTCTACAGTGTGAGCTGACGCTGTGTACGAAGATGGAGAAGCACCCCCAGAAGTTGCCTAAGTGTGTGCCTCCT
GACGAAGCCTGCACCTCGCTGGACGCCTCGATAATCTGGGCCATGATGCAGAATAAGAAGACGTTCACTAAGCCC
CTTGCTGTGATCCACCATGAAGCAGAATCTAAAGAAAAAGGTCCAAGCATGAAGGAACCAAATCCAATTTCTCCA

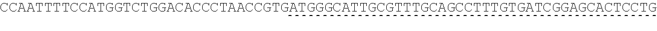

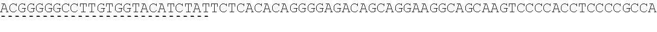

GCCTCGGAAAACAGCAGTGCTGCCCACAGCATCGGCAGCACGCAGAGCACGCCTTGCTCCAGCAGCAGCACGGCC

A nucleic acid sequence encoding a processed extracellular domain of betaglycan isoform B is shown below (SEQ ID NO: 592):

(SEQ ID NO: 592)
GGTCCAGAGCCTGGTGCACTGTGTGAACTGTCACCTGTCAGTGCCTCCCA
TCCTGTCCAGGCCTTGATGGAGAGCTTCACTGTTTTGTCAGGCTGTGCCA
GCAGAGGCACAACTGGGCTGCCACAGGAGGTGCATGTCCTGAATCTCCGC
ACTGCAGGCCAGGGGCCTGGCCAGCTACAGAGAGAGGTCACACTTCACCT
GAATCCCATCTCCTCAGTCCACATCCACCACAAGTCTGTTGTGTTCCTGC
TCAACTCCCCACACCCCCTGGTGTGGCATCTGAAGACAGAGAGACTTGCC
ACTGGGGTCTCCAGACTGTTTTTGGTGTCTGAGGGTTCTGTGGTCCAGTT
TTCATCAGCAAACTTCTCCTTGACAGCAGAAACAGAAGAAAGGAACTTCC
CCCATGGAAATGAACATCTGTTAAATTGGGCCCGAAAAGAGTATGGAGCA
GTTACTTCATTCACCGAACTCAAGATAGCAAGAAACATTTATATTAAAGT
GGGGGAAGATCAAGTGTTCCCTCCAAAGTGCAACATAGGGAAGAATTTTC
TCTCACTCAATTACCTTGCTGAGTACCTTCAACCCAAAGCAGCAGAAGGG
TGTGTGATGTCCAGCCAGCCCCAGAATGAGGAAGTACACATCATCGAGCT
AATCACCCCCAACTCTAACCCCTACAGTGCTTTCCAGGTGGATATAACAA
TTGATATAAGACCTTCTCAAGAGGATCTTGAAGTGGTCAAAAATCTCATC
CTGATCTTGAAGTGCAAAAAGTCTGTCAACTGGGTGATCAAATCTTTTGA
TGTTAAGGGAAGCCTGAAAATTATTGCTCCTAACAGTATTGGCTTTGGAA
AAGAGAGTGAAAGATCTATGACAATGACCAAATCAATAAGAGATGACATT
CCTTCAACCCAAGGGAATCTGGTGAAGTGGGCTTTGGACAATGGCTATAG
TCCAATAACTTCATACACAATGGCTCCTGTGGCTAATAGATTTCATCTTC
GGCTTGAAAATAATGAGGAGATGGGAGATGAGGAAGTCCACACTATTCCT
CCTGAGCTACGGATCCTGCTGGACCCTGGTGCCCTGCCTGCCCTGCAGAA
CCCGCCCATCCGGGGAGGGGAAGGCCAAAATGGAGGCCTTCCGTTTCCTT
TCCCAGATATTTCCAGGAGAGTCTGGAATGAAGAGGGAGAAGATGGGCTC
CCTCGGCCAAAGGACCCTGTCATTCCCAGCATACAACTGTTTCCTGGTCT
CAGAGAGCCAGAAGAGGTGCAAGGGAGCGTGGATATTGCCCTGTCTGTCA
AATGTGACAATGAGAAGATGATCGTGGCTGTAGAAAAAGATTCTTTTCAG
GCCAGTGGCTACTCGGGGATGGACGTCACCCTGTTGGATCCTACCTGCAA
GGCCAAGATGAATGGCACACACTTTGTTTTGGAGTCTCCTCTGAATGGCT
GCGGTACTCGGCCCCGGTGGTCAGCCCTTGATGGTGTGGTCTACTATAAC
TCCATTGTGATACAGGTTCCAGCCCTTGGGGACAGTAGTGGTTGGCCAGA
TGGTTATGAAGATCTGGAGTCAGGTGATAATGGATTTCCGGGAGATATGG
ATGAAGGAGATGCTTCCCTGTTCACCCGACCTGAAATCGTGGTGTTTAAT
TGCAGCCTTCAGCAGGTGAGGAACCCCAGCAGCTTCCAGGAACAGCCCCA
CGGAAACATCACCTTCAACATGGAGCTATACAACACTGACCTCTTTTTGG
TGCCCTCCCAGGGCGTCTTCTCTGTGCCAGAGAATGGACACGTTTATGTT
GAGGTATCTGTTACTAAGGCTGAACAAGAACTGGGATTTGCCATCCAAAC
GTGCTTTATCTCTCCATATTCGAACCCTGATAGGATGTCTCATTACACCA
TTATTGAGAATATTTGTCCTAAAGATGAATCTGTGAAATTCTACAGTCCC
AAGAGAGTGCACTTTCCTATCCCGCAAGCTGACATGGATAAGAAGCGATT
CAGCTTTGTCTTCAAGCCTGTCTTCAACACCTCACTGCTCTTTCTACAGT
GTGAGCTGACGCTGTGTACGAAGATGGAGAAGCACCCCCAGAAGTTGCCT
AAGTGTGTGCCTCCTGACGAAGCCTGCACCTCGCTGGACGCCTCGATAAT
CTGGGCCATGATGCAGAATAAGAAGACGTTCACTAAGCCCCTTGCTGTGA
TCCACCATGAAGCAGAATCTAAAGAAAAAGGTCCAAGCATGAAGGAACCA
AATCCAATTTCTCCACCAATTTTCCATGGTCTGGACACCCTAACCGTG

In certain embodiments, the disclosure relates to heteromultimers that comprise at least one betaglycan polypeptide, which includes fragments, functional variants, and modified forms thereof. Preferably, betaglycan polypeptides for use in accordance with inventions of the disclosure (e.g., heteromultimers comprising a betaglycan polypeptide and uses thereof) are soluble (e.g., an extracellular domain of betaglycan). In other preferred embodiments, betaglycan polypeptides for use in accordance with the inventions of the disclosure bind to and/or inhibit (antagonize) activity (e.g., Smad signaling) of one or more TGF-beta superfamily ligands. In some embodiments, heteromultimers of the disclosure comprise of at least one betaglycan polypeptide that is at least 70%, 75%, 80%, 85%, 90%, 91%, 92%, 93%, 94%, 95%, 96%, 97%, 98%, 99%, or 100% identical to the amino acid sequence of SEQ ID NOs: 585, 586, 589, or 590. In some embodiments, heteromultimers of the disclosure comprise at least one betaglycan polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 21-28 (e.g., amino acid residues 21, 22, 23, 24, 25, 26, 27, or 28) of SEQ ID NO: 585, and ends at any one of amino acids 381-787 (e.g., amino acid residues 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, or 787) of SEQ ID NO: 585. In some embodiments, heteromultimers of the disclosure comprise at least one betaglycan polypeptide that is at least 70%, 75%, 80%, 85%, 90%, 91%, 92%, 93%, 94%, 95%, 96%, 97%, 98%, 99%, or 100% identical to amino acids of 21-381 of SEQ ID NO: 585. In some embodiments, heteromultimers of the disclosure comprise at least one betaglycan polypeptide that is at least 70%, 75%, 80%, 85%, 90%, 91%, 92%, 93%, 94%, 95%, 96%, 97%, 98%, 99%, or 100% identical to amino acids of 21-787 of SEQ ID NO: 585. In some embodiments, heteromultimers of the disclosure comprise at least one betaglycan polypeptide that is at least 70%, 75%, 80%, 85%, 90%, 91%, 92%, 93%, 94%, 95%, 96%, 97%, 98%, 99%, or 100% identical to amino acids of 28-381 of SEQ ID NO: 585. In some embodiments, heteromultimers of the disclosure comprise at least one betaglycan polypeptide that is at least 70%, 75%, 80%, 85%, 90%, 91%, 92%, 93%, 94%, 95%, 96%, 97%, 98%, 99%, or 100% identical to amino acids of 28-787 of SEQ ID NO: 585. In some embodiments, heteromultimers of the disclosure comprise of at least one betaglycan polypeptide that is at least 70%, 75%, 80%, 85%, 90%, 91%, 92%, 93%, 94%, 95%, 96%, 97%, 98%, 99%, or 100% identical to amino acids of 21-781 of SEQ ID NO: 585. In some embodiments, heteromultimers of the disclosure comprise at least one betaglycan polypeptide that is at least 70%, 75%, 80%, 85%, 90%, 91%, 92%, 93%, 94%, 95%, 96%, 97%, 98%, 99%, or 100% identical to amino acids of 28-781 of SEQ ID NO: 585. In some embodiments, heteromultimers of the disclosure comprise at least one betaglycan polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 21-28 (e.g., amino acid residues 21, 22, 23, 24, 25, 26, or 27) of SEQ ID NO: 589, and ends at any one of amino acids 380-786 (e.g., amino acid residues 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, or 786) of SEQ ID NO: 589. In some embodiments, heteromultimers of the disclosure comprise at least one betaglycan polypeptide that is at least 70%, 75%, 80%, 85%, 90%, 91%, 92%, 93%, 94%, 95%, 96%, 97%, 98%, 99%, or 100% identical to amino acids of 21-380 of SEQ ID NO: 589. In some embodiments, heteromultimers of the disclosure comprise at least one betaglycan polypeptide that is at least 70%, 75%, 80%, 85%, 90%, 91%, 92%, 93%, 94%, 95%, 96%, 97%, 98%, 99%, or 100% identical to amino acids of 21-786 of SEQ ID NO: 589. In some embodiments, heteromultimers of the disclosure comprise at least one betaglycan polypeptide that is at least 70%, 75%, 80%, 85%, 90%, 91%, 92%, 93%, 94%, 95%, 96%, 97%, 98%, 99%, or 100% identical to amino acids of 28-380 of SEQ ID NO: 589. In some embodiments, heteromultimers of the disclosure comprise at least one betaglycan polypeptide that is at least 70%, 75%, 80%, 85%, 90%, 91%, 92%, 93%, 94%, 95%, 96%, 97%, 98%, 99%, or 100% identical to amino acids of 28-786 of SEQ ID NO: 589. In some embodiments, heteromultimers of the disclosure comprise at least one betaglycan polypeptide that is at least 70%, 75%, 80%, 85%, 90%, 91%, 92%, 93%, 94%, 95%, 96%, 97%, 98%, 99%, or 100% identical to amino acids of 21-780 of SEQ ID NO: 589. In some embodiments, heteromultimers of the disclosure comprise at least one betaglycan polypeptide that is at least 70%, 75%, 80%, 85%, 90%, 91%, 92%, 93%, 94%, 95%, 96%, 97%, 98%, 99%, or 100% identical to amino acids of 28-780 of SEQ ID NO: 589.

In some embodiments, heteromultimers of the disclosure comprise at least one betaglycan polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 21-28 (e.g., amino acid residues 21, 22, 23, 24, 25, 26, 27, or 28) of SEQ ID NO: 585, and ends at any one of amino acids 730-787 (e.g., amino acid residues 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, or 787) of SEQ ID NO: 585. In some embodiments, heteromultimers of the disclosure comprise at least one betaglycan polypeptide that is at least 70%, 75%, 80%, 85%, 90%, 91%, 92%, 93%, 94%, 95%, 96%, 97%, 98%, 99%, or 100% identical to amino acids of 21-787 of SEQ ID NO: 585. In some embodiments, heteromultimers of the disclosure comprise at least one betaglycan polypeptide that is at least 70%, 75%, 80%, 85%, 90%, 91%, 92%, 93%, 94%, 95%, 96%, 97%, 98%, 99%, or 100% identical to amino acids of 28-730 of SEQ ID NO: 585. In some embodiments, heteromultimers of the disclosure comprise at least one betaglycan polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 21-28 (e.g., amino acid residues 21, 22, 23, 24, 25, 26, 27, or 28) of SEQ ID NO: 585, and ends at any one of amino acids 730-787 (e.g., amino acid residues 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, or 787) of SEQ ID NO: 585. In some embodiments, heteromultimers of the disclosure comprise at least one betaglycan polypeptide that is at least 70%, 75%, 80%, 85%, 90%, 91%, 92%, 93%, 94%, 95%, 96%, 97%, 98%, 99%, or 100% identical to amino acids of 21-787 of SEQ ID NO: 585. In some embodiments, heteromultimers of the disclosure comprise at least one betaglycan polypeptide that is at least 70%, 75%, 80%, 85%, 90%, 91%, 92%, 93%, 94%, 95%, 96%, 97%, 98%, 99%, or 100% identical to amino acids of 28-730 of SEQ ID NO: 585. In some embodiments, heteromultimers of the disclosure comprise at least one betaglycan polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 21-28 (e.g., amino acid residues 21, 22, 23, 24, 25, 26, 27, or 28) of SEQ ID NO: 587, and ends at any one of amino acids 730-787 (e.g., amino acid residues 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, or 786) of SEQ ID NO: 587. In some embodiments, heteromultimers of the disclosure comprise at least one betaglycan polypeptide that is at least 70%, 75%, 80%, 85%, 90%, 91%, 92%, 93%, 94%, 95%, 96%, 97%, 98%, 99%, or 100% identical to amino acids of 21-786 of SEQ ID NO: 587. In some embodiments, heteromultimers of the disclosure comprise at least one betaglycan polypeptide that is at least 70%, 75%, 80%, 85%, 90%, 91%, 92%, 93%, 94%, 95%, 96%, 97%, 98%, 99%, or 100% identical to amino acids of 28-729 of SEQ ID NO: 587.

The term “MuSK polypeptide” includes polypeptides comprising any naturally occurring MuSK protein (encoded by MUSK or one of its nonhuman orthologs) as well as any variants thereof (including mutants, fragments, fusions, and peptidomimetic forms) that retain a useful activity.

A human MuSK isoform 1 precursor protein sequence (NCBI Reference Sequence NP_005583.1) is as follows:

| (SEQ ID NO: 595) | |
|---|---|
| 1 | MRELVNIPLV HILTLVAFSG TEKLPKAPVI TTPLETVDAL VEEVATFMCA |
| 51 | VESYPQPEIS WTRNKILIKL FDTRYSIREN GQLLTILSVE DSDDGIYCCT |
| 101 | ANNGVGGAVE SCGALQVKMK PKITRPPINV KIIEGLKAVL PCTTMGNPKP |
| 151 | SVSWIKGDSP LRENSRIAVL ESGSLRIHNV QKEDAGQYRC VAKNSLGTAY |
| 201 | SKVVKLEVEV FARILRAPES HNVTFGSFVT LHCTATGIPV PTITWIENGN |
| 251 | AVSSGSIQES VKDRVIDSRL QLFITKPGLY TCIATNKHGE KFSTAKAAAT |
| 301 | ISIAEWSKPQ KDNKGYCAQY RGEVCNAVLA KDALVFLNTS YADPEEAQEL |
| 351 | LVHTAWNELK VVSPVCRPAA EALLCNHIFQ ECSPGVVPTP IPICREYCLA |
| 401 | VKELFCAKEW LVMEEKTHRG LYRSEMHLLS VPECSKLPSM HWDPTACARL |
| 451 | 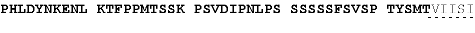 |
| 501 | 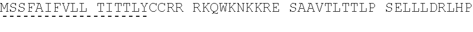 |
| 551 | NPMYQRMPLL LNPKLLSLEY PRNNIEYVRD IGEGAFGRVF QARAPGLLPY |
| 601 | EPFTMVAVKM LKEEASADMQ ADFQREAALM AEFDNPNIVK LLGVCAVGKP |
| 651 | MCLLFEYMAY GDLNEFLRSM SPHTVCSLSH SDLSMRAQVS SPGPPPLSCA |
| 701 | EQLCIARQVA AGMAYLSERK FVHRDLATRN CLVGENMVVK IADFGLSRNI |
| 751 | YSADYYKANE NDAIPIRWMP PESIFYNRYT TESDVWAYGV VLWEIFSYGL |
| 801 | QPYYGMAHEE VIYYVRDGNI LSCPENCPVE LYNLMRLCWS KLPADRPSFT |
| 851 | SIHRILERMC ERAEGTVSV |

The signal peptide is indicated by single underline, the extracellular domain is indicated in bold font, and the transmembrane domain is indicated by dotted underline. This isoform is the longest of human MuSK isoforms 1, 2, and 3.

A processed MuSK isoform 1 polypeptide sequence (SEQ ID NO: 596) is as follows:

| (SEQ ID NO: 596) | |
|---|---|
| 1 | GTEKLPKAPV ITTPLETVDA LVEEVATFMC AVESYPQPEI SWTRNKILIK |
| 51 | LFDTRYSIRE NGQLLTILSV EDSDDGIYCC TANNGVGGAV ESCGALQVKM |
| 101 | KPKITRPPIN VKIIEGLKAV LPCTTMGNPK PSVSWIKGDS PLRENSRIAV |
| 151 | LESGSLRIHN VQKEDAGQYR CVAKNSLGTA YSKVVKLEVE VFARILRAPE |
| 201 | SHNVTFGSFV TLHCTATGIP VPTITWIENG NAVSSGSIQE SVKDRVIDSR |
| 251 | LQLFITKPGL YTCIATNKHG EKFSTAKAAA TISIAEWSKP QKDNKGYCAQ |
| 301 | YRGEVCNAVL AKDALVFLNT SYADPEEAQE LLVHTAWNEL KVVSPVCRPA |
| 351 | AEALLCNHIF QECSPGVVPT PIPICREYCL AVKELFCAKE WLVMEEKTHR |
| 401 | GLYRSEMHLL SVPECSKLPS MHWDPTACAR LPHLDYNKEN LKTFPPMTSS |
| 451 | KPSVDIPNLP SSSSSSFSVS PTYSMT |

A nucleic acid sequence encoding the unprocessed precursor protein of human MuSK isoform 1 is shown below (SEQ ID NO: 597), corresponding to nucleotides 135-2744 of NCBI Reference Sequence NM_005592.3. The signal sequence is indicated by solid underline and the transmembrane region by dotted underline.

(SEQ ID NO: 597)
ATGAGAGAGCTCGTCAACATTCCACTGGTACATATTCTTACTCTGGTTGCCTTCAGCGGAACTGAGAAACTTCCA
AAAGCTCCTGTCATCACCACTCCTCTTGAAACAGTGGATGCCTTAGTTGAAGAAGTGGCTACTTTCATGTGTGCA
GTGGAATCCTACCCCCAGCCTGAGATTTCCTGGACTAGAAATAAAATTCTCATTAAACTCTTTGACACCCGGTAC
AGCATCCGGGAGAATGGGCAGCTCCTCACCATCCTGAGTGTGGAAGACAGTGATGATGGCATTTACTGCTGCACG
GCCAACAATGGTGTGGGAGGAGCTGTGGAGAGTTGTGGAGCCCTGCAAGTGAAGATGAAACCTAAAATAACTCGT
CCTCCCATAAATGTGAAAATAATAGAGGGATTAAAAGCAGTCCTACCATGTACTACAATGGGTAATCCCAAACCA
TCAGTGTCTTGGATAAAGGGAGACAGCCCTCTCAGGGAAAATTCCCGAATTGCAGTTCTTGAATCTGGGAGCTTG
AGGATTCATAACGTACAAAAGGAAGATGCAGGACAGTATCGATGTGTGGCAAAAAACAGCCTCGGGACAGCATAT
TCCAAAGTGGTGAAGCTGGAAGTTGAGGTTTTTGCCAGGATCCTGCGGGCTCCTGAATCCCACAATGTCACCTTT
GGCTCCTTTGTGACCCTGCACTGTACAGCAACAGGCATTCCTGTCCCCACCATCACCTGGATTGAAAACGGAAAT
GCTGTTTCTTCTGGGTCCATTCAAGAGAGTGTGAAAGACCGAGTGATTGACTCAAGACTGCAGCTGTTTATCACC
AAGCCAGGACTCTACACATGCATAGCTACCAATAAGCATGGGGAGAAGTTCAGTACTGCCAAGGCTGCAGCCACC
ATCAGCATAGCAGAATGGAGTAAACCACAGAAAGATAACAAAGGCTACTGCGCCCAGTACAGAGGGGAGGTGTGT
AATGCAGTCCTGGCAAAAGATGCTCTTGTTTTTCTCAACACCTCCTATGCGGACCCTGAGGAGGCCCAAGAGCTA
CTGGTCCACACGGCCTGGAATGAACTGAAAGTAGTGAGCCCAGTCTGCCGGCCAGCTGCTGAGGCTTTGTTGTGT
AACCACATCTTCCAGGAGTGCAGTCCTGGAGTAGTGCCTACTCCTATTCCCATTTGCAGAGAGTACTGCTTGGCA
GTAAAGGAGCTCTTCTGCGCAAAAGAATGGCTGGTAATGGAAGAGAAGACCCACAGAGGACTCTACAGATCCGAG
ATGCATTTGCTGTCCGTGCCAGAATGCAGCAAGCTTCCCAGCATGCATTGGGACCCCACGGCCTGTGCCAGACTG
CCACATCTAGATTATAACAAAGAAAACCTAAAAACATTCCCACCAATGACGTCCTCAAAGCCAAGTGTGGACATT

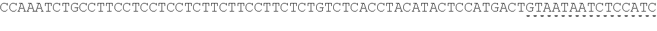

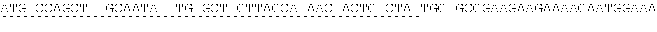

AATAAGAAAAGAGAATCAGCAGCAGTAACCCTCACCACACTGCCTTCTGAGCTCTTACTAGATAGACTTCATCCC
AACCCCATGTACCAGAGGATGCCGCTCCTTCTGAACCCCAAATTGCTCAGCCTGGAGTATCCAAGGAATAACATT
GAATATGTGAGAGACATCGGAGAGGGAGCGTTTGGAAGGGTGTTTCAAGCAAGGGCACCAGGCTTACTTCCCTAT
GAACCTTTCACTATGGTGGCAGTAAAGATGCTCAAAGAAGAAGCCTCGGCAGATATGCAAGCGGACTTTCAGAGG
GAGGCAGCCCTCATGGCAGAATTTGACAACCCTAACATTGTGAAGCTATTAGGAGTGTGTGCTGTCGGGAAGCCA
ATGTGCCTGCTCTTTGAATACATGGCCTATGGTGACCTCAATGAGTTCCTCCGCAGCATGTCCCCTCACACCGTG
TGCAGCCTCAGTCACAGTGACTTGTCTATGAGGGCTCAGGTCTCCAGCCCTGGGCCCCCACCCCTCTCCTGTGCT
GAGCAGCTTTGCATTGCCAGGCAGGTGGCAGCTGGCATGGCTTACCTCTCAGAACGTAAGTTTGTTCACCGAGAT
TTAGCCACCAGGAACTGCCTGGTGGGCGAGAACATGGTGGTGAAAATTGCCGACTTTGGCCTCTCCAGGAACATC
TACTCAGCAGACTACTACAAAGCTAATGAAAACGACGCTATCCCTATCCGTTGGATGCCACCAGAGTCCATTTTT
TATAACCGCTACACTACAGAGTCTGATGTGTGGGCCTATGGCGTGGTCCTCTGGGAGATCTTCTCCTATGGCCTG
CAGCCCTACTATGGGATGGCCCATGAGGAGGTCATTTACTACGTGCGAGATGGCAACATCCTCTCCTGCCCTGAG
AACTGCCCCGTGGAGCTGTACAATCTCATGCGTCTATGTTGGAGCAAGCTGCCTGCAGACAGACCCAGTTTCACC
AGTATTCACCGAATTCTGGAACGCATGTGTGAGAGGGCAGAGGGAACTGTGAGTGTC

A nucleic acid sequence encoding a processed extracellular domain of MuSK isoform 1 is shown below (SEQ ID NO: 598):

(SEQ ID NO: 598)
GGAACTGAGAAACTTCCAAAAGCTCCTGTCATCACCACTCCTCTTGAAAC
AGTGGATGCCTTAGTTGAAGAAGTGGCTACTTTCATGTGTGCAGTGGAAT
CCTACCCCCAGCCTGAGATTTCCTGGACTAGAAATAAAATTCTCATTAAA
CTCTTTGACACCCGGTACAGCATCCGGGAGAATGGGCAGCTCCTCACCAT
CCTGAGTGTGGAAGACAGTGATGATGGCATTTACTGCTGCACGGCCAACA
ATGGTGTGGGAGGAGCTGTGGAGAGTTGTGGAGCCCTGCAAGTGAAGATG
AAACCTAAAATAACTCGTCCTCCCATAAATGTGAAAATAATAGAGGGATT
AAAAGCAGTCCTACCATGTACTACAATGGGTAATCCCAAACCATCAGTGT
CTTGGATAAAGGGAGACAGCCCTCTCAGGGAAAATTCCCGAATTGCAGTT
CTTGAATCTGGGAGCTTGAGGATTCATAACGTACAAAAGGAAGATGCAGG
ACAGTATCGATGTGTGGCAAAAAACAGCCTCGGGACAGCATATTCCAAAG
TGGTGAAGCTGGAAGTTGAGGTTTTTGCCAGGATCCTGCGGGCTCCTGAA
TCCCACAATGTCACCTTTGGCTCCTTTGTGACCCTGCACTGTACAGCAAC
AGGCATTCCTGTCCCCACCATCACCTGGATTGAAAACGGAAATGCTGTTT
CTTCTGGGTCCATTCAAGAGAGTGTGAAAGACCGAGTGATTGACTCAAGA
CTGCAGCTGTTTATCACCAAGCCAGGACTCTACACATGCATAGCTACCAA
TAAGCATGGGGAGAAGTTCAGTACTGCCAAGGCTGCAGCCACCATCAGCA
TAGCAGAATGGAGTAAACCACAGAAAGATAACAAAGGCTACTGCGCCCAG
TACAGAGGGGAGGTGTGTAATGCAGTCCTGGCAAAAGATGCTCTTGTTTT
TCTCAACACCTCCTATGCGGACCCTGAGGAGGCCCAAGAGCTACTGGTCC
ACACGGCCTGGAATGAACTGAAAGTAGTGAGCCCAGTCTGCCGGCCAGCT
GCTGAGGCTTTGTTGTGTAACCACATCTTCCAGGAGTGCAGTCCTGGAGT
AGTGCCTACTCCTATTCCCATTTGCAGAGAGTACTGCTTGGCAGTAAAGG
AGCTCTTCTGCGCAAAAGAATGGCTGGTAATGGAAGAGAAGACCCACAGA
GGACTCTACAGATCCGAGATGCATTTGCTGTCCGTGCCAGAATGCAGCAA
GCTTCCCAGCATGCATTGGGACCCCACGGCCTGTGCCAGACTGCCACATC
TAGATTATAACAAAGAAAACCTAAAAACATTCCCACCAATGACGTCCTCA
AAGCCAAGTGTGGACATTCCAAATCTGCCTTCCTCCTCCTCTTCTTCCTT
CTCTGTCTCACCTACATACTCCATGACT

A human MuSK isoform 2 precursor protein sequence (NCBI Reference Sequence NP_001159752.1) is as follows:

| (SEQ ID NO: 599) | |
|---|---|
| 1 | MRELVNIPLV HILTLVAFSG TEKLPKAPVI TTPLETVDAL VEEVATFMCA |
| 51 | VESYPQPEIS WTRNKILIKL FDTRYSIREN GQLLTILSVE DSDDGIYCCT |
| 101 | ANNGVGGAVE SCGALQVKMK PKITRPPINV KIIEGLKAVL PCTTMGNPKP |
| 151 | SVSWIKGDSP LRENSRIAVL ESGSLRIHNV QKEDAGQYRC VAKNSLGTAY |
| 201 | SKVVKLEVEE ESEPEQDTKV FARILRAPES HNVTFGSFVT LHCTATGIPV |
| 251 | PTITWIENGN AVSSGSIQES VEDRVIDSRL QLFITKPGLY TCIATNKHGE |
| 301 | KFSTAKAAAT ISIAEWREYC LAVKELFCAK EWLVMEEKTH RGLYRSEMHL |
| 351 | LSVPECSKLP SMHWDPTACA RLPHLAFPPM TSSKPSVDIP NLPSSSSSSF |
| 401 | 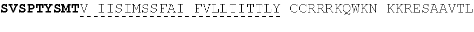 |
| 451 | TTLPSELLLD RLHPNPMYQR MPLLLNPKLL SLEYPRNNIE YVRDIGEGAF |
| 501 | GRVFQARAPG LLPYEPFTMV AVKMLKEEAS ADMQADFQRE AALMAEFDNP |
| 551 | NIVKLLGVCA VGKPMCLLFE YMAYGDLNEF LRSMSPHTVC SLSHSDLSMR |
| 601 | AQVSSPGPPP LSCAEQLCIA RQVAAGMAYL SERKFVHRDL ATRNCLVGEN |
| 651 | MVVKIADFGL SRNIYSADYY KANENDAIPI RWMPPESIFY NRYTTESDVW |
| 701 | AYGVVLWEIF SYGLQPYYGM AHEEVIYYVR DGNILSCPEN CPVELYNLMR |
| 751 | LCWSKLPADR PSFTSIHRIL ERMCERAEGT VSV |

The signal peptide is indicated by single underline, the extracellular domain is indicated in bold font, and the transmembrane domain is indicated by dotted underline. This variant contains an alternate in-frame exon and lacks an alternate in-frame exon in the middle portion of the coding region compared to variant 1. The encoded isoform 2 is shorter than isoform 1.

A mature MuSK isoform 2 polypeptide sequence (SEQ ID NO: 600) is as follows:

| (SEQ ID NO: 600) | |
|---|---|
| 1 | GTEKLPKAPV ITTPLETVDA LVEEVATFMC AVESYPQPEI SWTRNKILIK |
| 51 | LFDTRYSIRE NGQLLTILSV EDSDDGIYCC TANNGVGGAV ESCGALQVKM |
| 101 | KPKITRPPIN VKIIEGLKAV LPCTTMGNPK PSVSWIKGDS PLRENSRIAV |
| 151 | LESGSLRIHN VQKEDAGQYR CVAKNSLGTA YSKVVKLEVE EESEPEQDTK |
| 201 | VFARILRAPE SHNVTFGSFV TLHCTATGIP VPTITWIENG NAVSSGSIQE |
| 251 | SVKDRVIDSR LQLFITKPGL YTCIATNKHG EKFSTAKAAA TISIAEWREY |
| 301 | CLAVKELFCA KEWLVMEEKT HRGLYRSEMH LLSVPECSKL PSMHWDPTAC |
| 351 | ARLPHLAFPP MTSSKPSVDI PNLPSSSSSS FSVSPTYSMT |

A nucleic acid sequence encoding the unprocessed precursor protein of human MuSK isoform 2 is shown below (SEQ ID NO: 601), corresponding to nucleotides 135-2483 of NCBI Reference Sequence NM_001166280.1. The signal sequence is indicated by solid underline and the transmembrane region by dotted underline.

(SEQ ID NO: 601)
ATGAGAGAGCTCGTCAACATTCCACTGGTACATATTCTTACTCTGGTTGCCTTCAGCGGAACTGAGAAACTTCCA
AAAGCTCCTGTCATCACCACTCCTCTTGAAACAGTGGATGCCTTAGTTGAAGAAGTGGCTACTTTCATGTGTGCA
GTGGAATCCTACCCCCAGCCTGAGATTTCCTGGACTAGAAATAAAATTCTCATTAAACTCTTTGACACCCGGTAC
AGCATCCGGGAGAATGGGCAGCTCCTCACCATCCTGAGTGTGGAAGACAGTGATGATGGCATTTACTGCTGCACG
GCCAACAATGGTGTGGGAGGAGCTGTGGAGAGTTGTGGAGCCCTGCAAGTGAAGATGAAACCTAAAATAACTCGT
CCTCCCATAAATGTGAAAATAATAGAGGGATTAAAAGCAGTCCTACCATGTACTACAATGGGTAATCCCAAACCA
TCAGTGTCTTGGATAAAGGGAGACAGCCCTCTCAGGGAAAATTCCCGAATTGCAGTTCTTGAATCTGGGAGCTTG
AGGATTCATAACGTACAAAAGGAAGATGCAGGACAGTATCGATGTGTGGCAAAAAACAGCCTCGGGACAGCATAT
TCCAAAGTGGTGAAGCTGGAAGTTGAGGAAGAAAGTGAACCCGAACAAGATACTAAAGTTTTTGCCAGGATCCTG
CGGGCTCCTGAATCCCACAATGTCACCTTTGGCTCCTTTGTGACCCTGCACTGTACAGCAACAGGCATTCCTGTC
CCCACCATCACCTGGATTGAAAACGGAAATGCTGTTTCTTCTGGGTCCATTCAAGAGAGTGTGAAAGACCGAGTG
ATTGACTCAAGACTGCAGCTGTTTATCACCAAGCCAGGACTCTACACATGCATAGCTACCAATAAGCATGGGGAG
AAGTTCAGTACTGCCAAGGCTGCAGCCACCATCAGCATAGCAGAATGGAGAGAGTACTGCTTGGCAGTAAAGGAG
CTCTTCTGCGCAAAAGAATGGCTGGTAATGGAAGAGAAGACCCACAGAGGACTCTACAGATCCGAGATGCATTTG
CTGTCCGTGCCAGAATGCAGCAAGCTTCCCAGCATGCATTGGGACCCCACGGCCTGTGCCAGACTGCCACATCTA
GCATTCCCACCAATGACGTCCTCAAAGCCAAGTGTGGACATTCCAAATCTGCCTTCCTCCTCCTCTTCTTCCTTC

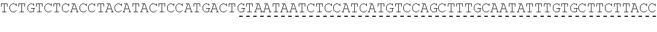

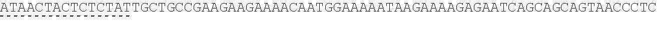

ACCACACTGCCTTCTGAGCTCTTACTAGATAGACTTCATCCCAACCCCATGTACCAGAGGATGCCGCTCCTTCTG
AACCCCAAATTGCTCAGCCTGGAGTATCCAAGGAATAACATTGAATATGTGAGAGACATCGGAGAGGGAGCGTTT
GGAAGGGTGTTTCAAGCAAGGGCACCAGGCTTACTTCCCTATGAACCTTTCACTATGGTGGCAGTAAAGATGCTC
AAAGAAGAAGCCTCGGCAGATATGCAAGCGGACTTTCAGAGGGAGGCAGCCCTCATGGCAGAATTTGACAACCCT
AACATTGTGAAGCTATTAGGAGTGTGTGCTGTCGGGAAGCCAATGTGCCTGCTCTTTGAATACATGGCCTATGGT
GACCTCAATGAGTTCCTCCGCAGCATGTCCCCTCACACCGTGTGCAGCCTCAGTCACAGTGACTTGTCTATGAGG
GCTCAGGTCTCCAGCCCTGGGCCCCCACCCCTCTCCTGTGCTGAGCAGCTTTGCATTGCCAGGCAGGTGGCAGCT
GGCATGGCTTACCTCTCAGAACGTAAGTTTGTTCACCGAGATTTAGCCACCAGGAACTGCCTGGTGGGCGAGAAC
ATGGTGGTGAAAATTGCCGACTTTGGCCTCTCCAGGAACATCTACTCAGCAGACTACTACAAAGCTAATGAAAAC
GACGCTATCCCTATCCGTTGGATGCCACCAGAGTCCATTTTTTATAACCGCTACACTACAGAGTCTGATGTGTGG
GCCTATGGCGTGGTCCTCTGGGAGATCTTCTCCTATGGCCTGCAGCCCTACTATGGGATGGCCCATGAGGAGGTC
ATTTACTACGTGCGAGATGGCAACATCCTCTCCTGCCCTGAGAACTGCCCCGTGGAGCTGTACAATCTCATGCGT
CTATGTTGGAGCAAGCTGCCTGCAGACAGACCCAGTTTCACCAGTATTCACCGAATTCTGGAACGCATGTGTGAG
AGGGCAGAGGGAACTGTGAGTGTC

A nucleic acid sequence encoding a processed extracellular domain of MuSK isoform 2 is shown below (SEQ ID NO: 602):

(SEQ ID NO: 602)
GGAACTGAGAAACTTCCAAAAGCTCCTGTCATCACCACTCCTCTTGAAAC
AGTGGATGCCTTAGTTGAAGAAGTGGCTACTTTCATGTGTGCAGTGGAAT
CCTACCCCCAGCCTGAGATTTCCTGGACTAGAAATAAAATTCTCATTAAA
CTCTTTGACACCCGGTACAGCATCCGGGAGAATGGGCAGCTCCTCACCAT
CCTGAGTGTGGAAGACAGTGATGATGGCATTTACTGCTGCACGGCCAACA
ATGGTGTGGGAGGAGCTGTGGAGAGTTGTGGAGCCCTGCAAGTGAAGATG
AAACCTAAAATAACTCGTCCTCCCATAAATGTGAAAATAATAGAGGGATT
AAAAGCAGTCCTACCATGTACTACAATGGGTAATCCCAAACCATCAGTGT
CTTGGATAAAGGGAGACAGCCCTCTCAGGGAAAATTCCCGAATTGCAGTT
CTTGAATCTGGGAGCTTGAGGATTCATAACGTACAAAAGGAAGATGCAGG
ACAGTATCGATGTGTGGCAAAAAACAGCCTCGGGACAGCATATTCCAAAG
TGGTGAAGCTGGAAGTTGAGGAAGAAAGTGAACCCGAACAAGATACTAAA
GTTTTTGCCAGGATCCTGCGGGCTCCTGAATCCCACAATGTCACCTTTGG
CTCCTTTGTGACCCTGCACTGTACAGCAACAGGCATTCCTGTCCCCACCA
TCACCTGGATTGAAAACGGAAATGCTGTTTCTTCTGGGTCCATTCAAGAG
AGTGTGAAAGACCGAGTGATTGACTCAAGACTGCAGCTGTTTATCACCAA
GCCAGGACTCTACACATGCATAGCTACCAATAAGCATGGGGAGAAGTTCA
GTACTGCCAAGGCTGCAGCCACCATCAGCATAGCAGAATGGAGAGAGTAC
TGCTTGGCAGTAAAGGAGCTCTTCTGCGCAAAAGAATGGCTGGTAATGGA
AGAGAAGACCCACAGAGGACTCTACAGATCCGAGATGCATTTGCTGTCCG
TGCCAGAATGCAGCAAGCTTCCCAGCATGCATTGGGACCCCACGGCCTGT
GCCAGACTGCCACATCTAGCATTCCCACCAATGACGTCCTCAAAGCCAAG
TGTGGACATTCCAAATCTGCCTTCCTCCTCCTCTTCTTCCTTCTCTGTCT
CACCTACATACTCCATGACT

A human MuSK isoform 3 precursor protein sequence (NCBI Reference Sequence NP_001159753.1) is as follows:

| (SEQ ID NO: 603) | |
|---|---|
| 1 | MRELVNIPLV HILTLVAFSG TEKLPKAPVI TTPLETVDAL VEEVATFMCA |
| 51 | VESYPQPEIS WTRNKILIKL FDTRYSIREN GQLLTILSVE DSDDGIYCCT |
| 101 | ANNGVGGAVE SCGALQVKMK PKITRPPINV KIIEGLKAVL PCTTMGNPKP |
| 151 | SVSWIKGDSP LRENSRIAVL ESGSLRIHNV QKEDAGQYRC VAKNSLGTAY |
| 201 | SKVVKLEVEV FARILRAPES HNVTFGSFVT LHCTATGIPV PTITWIENGN |
| 251 | AVSSGSIQES VKDRVIDSRL QLFITKPGLY TCIATNKHGE KFSTAKAAAT |
| 301 | ISIAEWREYC LAVKELFCAK EWLVMEEKTH RGLYRSEMHL LSVPECSKLP |
| 351 | 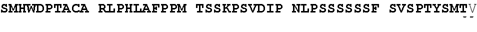 |
| 401 | 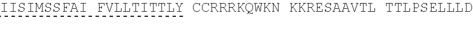 |
| 451 | RLHPNPMYQR MPLLLNPKLL SLEYPRNNIE YVRDIGEGAF GRVFQARAPG |
| 501 | LLPYEPFTMV AVKMLKEEAS ADMQADFQRE AALMAEFDNP NIVKLLGVCA |
| 551 | VGKPMCLLFE YMAYGDLNEF LRSMSPHTVC SLSHSDLSMR AQVSSPGPPP |
| 601 | LSCAEQLCIA RQVAAGMAYL SERKFVHRDL ATRNCLVGEN MVVKIADFGL |
| 651 | SRNIYSADYY KANENDAIPI RWMPPESIFY NRYTTESDVW AYGVVLWEIF |
| 701 | SYGLQPYYGM AHEEVIYYVR DGNILSCPEN CPVELYNLMR LCWSKLPADR |
| 751 | PSFTSIHRIL ERMCERAEGT VSV |

The signal peptide is indicated by single underline, the extracellular domain is indicated in bold font, and the transmembrane domain is indicated by dotted underline. This variant lacks an alternate in-frame exon in the middle portion of the coding region compared to variant 1. The encoded isoform 3 is shorter than isoform 1.

A processed MuSK isoform 3 polypeptide sequence (SEQ ID NO: 604) is as follows:

| (SEQ ID NO: 604) | |
|---|---|
| 1 | GTEKLPKAPV ITTPLETVDA LVEEVATFMC AVESYPQPEI SWTRNKILIK |
| 51 | LFDTRYSIRE NGQLLTILSV EDSDDGIYCC TANNGVGGAV ESCGALQVKM |
| 101 | KPKITRPPIN VKIIEGLKAV LPCTTMGNPK PSVSWIKGDS PLRENSRIAV |
| 151 | LESGSLRIHN VQKEDAGQYR CVAKNSLGTA YSKVVKLEVE VFARILRAPE |
| 201 | SHNVTFGSFV TLHCTATGIP VPTITWIENG NAVSSGSIQE SVKDRVIDSR |
| 251 | LQLFITKPGL YTCIATNKHG EKFSTAKAAA TISIAEWREY CLAVKELFCA |
| 301 | KEWLVMEEKT HRGLYRSEMH LLSVPECSKL PSMHWDPTAC ARLPHLAFPP |
| 351 | MTSSKPSVDI PNLPSSSSSS FSVSPTYSMT |

A nucleic acid sequence encoding the unprocessed precursor protein of human MuSK isoform 3 is shown below (SEQ ID NO: 605), corresponding to nucleotides 135-2453 of NCBI Reference Sequence NM_001166281.1. The signal sequence is indicated by solid underline and the transmembrane region by dotted underline.

(SEQ ID NO: 605)
ATGAGAGAGCTCGTCAACATTCCACTGGTACATATTCTTACTCTGGTTGCCTTCAGCGGAACTGAGAAACTTCCA
AAAGCTCCTGTCATCACCACTCCTCTTGAAACAGTGGATGCCTTAGTTGAAGAAGTGGCTACTTTCATGTGTGCA
GTGGAATCCTACCCCCAGCCTGAGATTTCCTGGACTAGAAATAAAATTCTCATTAAACTCTTTGACACCCGGTAC
AGCATCCGGGAGAATGGGCAGCTCCTCACCATCCTGAGTGTGGAAGACAGTGATGATGGCATTTACTGCTGCACG
GCCAACAATGGTGTGGGAGGAGCTGTGGAGAGTTGTGGAGCCCTGCAAGTGAAGATGAAACCTAAAATAACTCGT
CCTCCCATAAATGTGAAAATAATAGAGGGATTAAAAGCAGTCCTACCATGTACTACAATGGGTAATCCCAAACCA
TCAGTGTCTTGGATAAAGGGAGACAGCCCTCTCAGGGAAAATTCCCGAATTGCAGTTCTTGAATCTGGGAGCTTG
AGGATTCATAACGTACAAAAGGAAGATGCAGGACAGTATCGATGTGTGGCAAAAAACAGCCTCGGGACAGCATAT
TCCAAAGTGGTGAAGCTGGAAGTTGAGGTTTTTGCCAGGATCCTGCGGGCTCCTGAATCCCACAATGTCACCTTT
GGCTCCTTTGTGACCCTGCACTGTACAGCAACAGGCATTCCTGTCCCCACCATCACCTGGATTGAAAACGGAAAT
GCTGTTTCTTCTGGGTCCATTCAAGAGAGTGTGAAAGACCGAGTGATTGACTCAAGACTGCAGCTGTTTATCACC
AAGCCAGGACTCTACACATGCATAGCTACCAATAAGCATGGGGAGAAGTTCAGTACTGCCAAGGCTGCAGCCACC
ATCAGCATAGCAGAATGGAGAGAGTACTGCTTGGCAGTAAAGGAGCTCTTCTGCGCAAAAGAATGGCTGGTAATG
GAAGAGAAGACCCACAGAGGACTCTACAGATCCGAGATGCATTTGCTGTCCGTGCCAGAATGCAGCAAGCTTCCC
AGCATGCATTGGGACCCCACGGCCTGTGCCAGACTGCCACATCTAGCATTCCCACCAATGACGTCCTCAAAGCCA

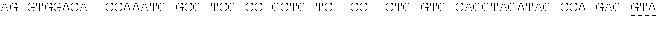

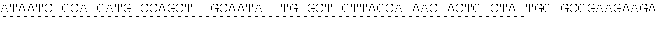

AAACAATGGAAAAATAAGAAAAGAGAATCAGCAGCAGTAACCCTCACCACACTGCCTTCTGAGCTCTTACTAGAT
AGACTTCATCCCAACCCCATGTACCAGAGGATGCCGCTCCTTCTGAACCCCAAATTGCTCAGCCTGGAGTATCCA
AGGAATAACATTGAATATGTGAGAGACATCGGAGAGGGAGCGTTTGGAAGGGTGTTTCAAGCAAGGGCACCAGGC
TTACTTCCCTATGAACCTTTCACTATGGTGGCAGTAAAGATGCTCAAAGAAGAAGCCTCGGCAGATATGCAAGCG
GACTTTCAGAGGGAGGCAGCCCTCATGGCAGAATTTGACAACCCTAACATTGTGAAGCTATTAGGAGTGTGTGCT
GTCGGGAAGCCAATGTGCCTGCTCTTTGAATACATGGCCTATGGTGACCTCAATGAGTTCCTCCGCAGCATGTCC
CCTCACACCGTGTGCAGCCTCAGTCACAGTGACTTGTCTATGAGGGCTCAGGTCTCCAGCCCTGGGCCCCCACCC
CTCTCCTGTGCTGAGCAGCTTTGCATTGCCAGGCAGGTGGCAGCTGGCATGGCTTACCTCTCAGAACGTAAGTTT
GTTCACCGAGATTTAGCCACCAGGAACTGCCTGGTGGGCGAGAACATGGTGGTGAAAATTGCCGACTTTGGCCTC
TCCAGGAACATCTACTCAGCAGACTACTACAAAGCTAATGAAAACGACGCTATCCCTATCCGTTGGATGCCACCA
GAGTCCATTTTTTATAACCGCTACACTACAGAGTCTGATGTGTGGGCCTATGGCGTGGTCCTCTGGGAGATCTTC
TCCTATGGCCTGCAGCCCTACTATGGGATGGCCCATGAGGAGGTCATTTACTACGTGCGAGATGGCAACATCCTC
TCCTGCCCTGAGAACTGCCCCGTGGAGCTGTACAATCTCATGCGTCTATGTTGGAGCAAGCTGCCTGCAGACAGA
CCCAGTTTCACCAGTATTCACCGAATTCTGGAACGCATGTGTGAGAGGGCAGAGGGAACTGTGAGTGTCTAA

A nucleic acid sequence encoding a processed extracellular domain of MuSK isoform 3 is shown below (SEQ ID NO: 606):

(SEQ ID NO: 606)
GGAACTGAGAAACTTCCAAAAGCTCCTGTCATCACCACTCCTCTTGAAAC
AGTGGATGCCTTAGTTGAAGAAGTGGCTACTTTCATGTGTGCAGTGGAAT
CCTACCCCCAGCCTGAGATTTCCTGGACTAGAAATAAAATTCTCATTAAA
CTCTTTGACACCCGGTACAGCATCCGGGAGAATGGGCAGCTCCTCACCAT
CCTGAGTGTGGAAGACAGTGATGATGGCATTTACTGCTGCACGGCCAACA
ATGGTGTGGGAGGAGCTGTGGAGAGTTGTGGAGCCCTGCAAGTGAAGATG
AAACCTAAAATAACTCGTCCTCCCATAAATGTGAAAATAATAGAGGGATT
AAAAGCAGTCCTACCATGTACTACAATGGGTAATCCCAAACCATCAGTGT
CTTGGATAAAGGGAGACAGCCCTCTCAGGGAAAATTCCCGAATTGCAGTT
CTTGAATCTGGGAGCTTGAGGATTCATAACGTACAAAAGGAAGATGCAGG
ACAGTATCGATGTGTGGCAAAAAACAGCCTCGGGACAGCATATTCCAAAG
TGGTGAAGCTGGAAGTTGAGGTTTTTGCCAGGATCCTGCGGGCTCCTGAA
TCCCACAATGTCACCTTTGGCTCCTTTGTGACCCTGCACTGTACAGCAAC
AGGCATTCCTGTCCCCACCATCACCTGGATTGAAAACGGAAATGCTGTTT
CTTCTGGGTCCATTCAAGAGAGTGTGAAAGACCGAGTGATTGACTCAAGA
CTGCAGCTGTTTATCACCAAGCCAGGACTCTACACATGCATAGCTACCAA
TAAGCATGGGGAGAAGTTCAGTACTGCCAAGGCTGCAGCCACCATCAGCA
TAGCAGAATGGAGAGAGTACTGCTTGGCAGTAAAGGAGCTCTTCTGCGCA
AAAGAATGGCTGGTAATGGAAGAGAAGACCCACAGAGGACTCTACAGATC
CGAGATGCATTTGCTGTCCGTGCCAGAATGCAGCAAGCTTCCCAGCATGC
ATTGGGACCCCACGGCCTGTGCCAGACTGCCACATCTAGCATTCCCACCA
ATGACGTCCTCAAAGCCAAGTGTGGACATTCCAAATCTGCCTTCCTCCTC
CTCTTCTTCCTTCTCTGTCTCACCTACATACTCCATGACT

In certain embodiments, the disclosure relates to heteromultimers that comprise at least one MuSK polypeptide, which includes fragments, functional variants, and modified forms thereof. Preferably, MuSK polypeptides for use in accordance with the disclosure (e.g., heteromultimers comprising a MuSK polypeptide and uses thereof) are soluble (e.g., an extracellular domain of MuSK). In other preferred embodiments, MuSK polypeptides for use in accordance with disclosure bind to and/or inhibit (antagonize) activity (e.g., Smad signaling) of one or more TGF-beta superfamily ligands. In some embodiments, heteromultimers of the disclosure comprise at least one MuSK polypeptide that is at least 70%, 75%, 80%, 85%, 90%, 91%, 92%, 93%, 94%, 95%, 96%, 97%, 98%, 99%, or 100% identical to the amino acid sequence of SEQ ID NOs: 595, 596, 598, 599, 600, 603, and 604. In some embodiments, heteromultimers of the disclosure comprise at least one MuSK polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 21-49 (e.g., amino acid residues 21, 22, 23, 24, 25, 26, 27, 28, 29, 30, 31, 32, 33, 34, 35, 35, 36, 37, 38, 39, 40, 41, 42, 43, 44, 45, 46, 47, 48, or 49) of SEQ ID NO: 595, and ends at any one of amino acids 447-495 (e.g., amino acid residues 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, or 495) of SEQ ID NO: 595. In some embodiments, heteromultimers of the disclosure comprise of at least one MuSK polypeptide that is at least 70%, 75%, 80%, 85%, 90%, 91%, 92%, 93%, 94%, 95%, 96%, 97%, 98%, 99%, or 100% identical to amino acids of 21-495 of SEQ ID NO: 595. In some embodiments, heteromultimers of the disclosure comprise of at least one MuSK polypeptide that is at least 70%, 75%, 80%, 85%, 90%, 91%, 92%, 93%, 94%, 95%, 96%, 97%, 98%, 99%, or 100% identical to amino acids of 49-447 of SEQ ID NO: 595. In some embodiments, heteromultimers of the disclosure comprise of at least one MuSK polypeptide that is at least 70%, 75%, 80%, 85%, 90%, 91%, 92%, 93%, 94%, 95%, 96%, 97%, 98%, 99%, or 100% identical to amino acids of 210-495 of SEQ ID NO: 595. In some embodiments, heteromultimers of the disclosure comprise at least one MuSK polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 20-49 (e.g., amino acid residues 20, 21, 22, 23, 24, 25, 26, 27, 28, 29, 30, 31, 32, 33, 34, 35, 35, 36, 37, 38, 39, 40, 41, 42, 43, 44, 45, 46, 47, 48, or 49) of SEQ ID NO: 599, and ends at any one of amino acids 369-409 (e.g., amino acid residues 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, or 409) of SEQ ID NO: 599. In some embodiments, heteromultimers of the disclosure comprise of at least one MuSK polypeptide that is at least 70%, 75%, 80%, 85%, 90%, 91%, 92%, 93%, 94%, 95%, 96%, 97%, 98%, 99%, or 100% identical to amino acids of 20-409 of SEQ ID NO: 599. In some embodiments, heteromultimers of the disclosure comprise of at least one MuSK polypeptide that is at least 70%, 75%, 80%, 85%, 90%, 91%, 92%, 93%, 94%, 95%, 96%, 97%, 98%, 99%, or 100% identical to amino acids of 49-369 of SEQ ID NO: 599. In some embodiments, heteromultimers of the disclosure comprise of at least one MuSK polypeptide that is at least 70%, 75%, 80%, 85%, 90%, 91%, 92%, 93%, 94%, 95%, 96%, 97%, 98%, 99%, or 100% identical to amino acids of 210-409 of SEQ ID NO: 599. In some embodiments, heteromultimers of the disclosure comprise at least one MuSK polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 20-49 (e.g., amino acid residues 20, 21, 22, 23, 24, 25, 26, 27, 28, 29, 30, 31, 32, 33, 34, 35, 35, 36, 37, 38, 39, 40, 41, 42, 43, 44, 45, 46, 47, 48, or 49) of SEQ ID NO: 603, and ends at any one of amino acids 359-399 (e.g., amino acid residues 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, or 399) of SEQ ID NO: 603. In some embodiments, heteromultimers of the disclosure comprise of at least one MuSK polypeptide that is at least 70%, 75%, 80%, 85%, 90%, 91%, 92%, 93%, 94%, 95%, 96%, 97%, 98%, 99%, or 100% identical to amino acids of 20-399 of SEQ ID NO: 603. In some embodiments, heteromultimers of the disclosure comprise of at least one MuSK polypeptide that is at least 70%, 75%, 80%, 85%, 90%, 91%, 92%, 93%, 94%, 95%, 96%, 97%, 98%, 99%, or 100% identical to amino acids of 49-359 of SEQ ID NO: 603. In some embodiments, heteromultimers of the disclosure comprise of at least one MuSK polypeptide that is at least 70%, 75%, 80%, 85%, 90%, 91%, 92%, 93%, 94%, 95%, 96%, 97%, 98%, 99%, or 100% identical to amino acids of 210-399 of SEQ ID NO: 603.

In some embodiments, the present disclosure contemplates making functional variants by modifying the structure of a TGF-beta superfamily type I receptor polypeptide (e.g., ALK1, ALK2, ALK3, ALK4, ALK5, ALK6, and ALK7), a TGF-beta superfamily type II receptor polypeptide (e.g., ActRIIA, ActRIIB, TGFBRII, BMPRII, and MISRII), and/or a TGF-beta superfamily co-receptor (e.g., endoglin, betaglycan, Cripto-1, Cryptic, Cryptic family protein 1B, CRIM1, CRIM2, BAMBI, BMPER, RGM-A, RGM-B, hemojuvelin, and MuSK) for such purposes as enhancing therapeutic efficacy or stability (e.g., shelf-life and resistance to proteolytic degradation in vivo). Variants can be produced by amino acid substitution, deletion, addition, or combinations thereof. For instance, it is reasonable to expect that an isolated replacement of a leucine with an isoleucine or valine, an aspartate with a glutamate, a threonine with a serine, or a similar replacement of an amino acid with a structurally related amino acid (e.g., conservative mutations) will not have a major effect on the biological activity of the resulting molecule. Conservative replacements are those that take place within a family of amino acids that are related in their side chains. Whether a change in the amino acid sequence of a polypeptide of the disclosure results in a functional homolog can be readily determined by assessing the ability of the variant polypeptide to produce a response in cells in a fashion similar to the wild-type polypeptide, or to bind to one or more TGF-beta superfamily ligands including, for example, BMP2, BMP2/7, BMP3, BMP4, BMP4/7, BMP5, BMP6, BMP7, BMP8a, BMP8b, BMP9, BMP10, GDF3, GDF5, GDF6/BMP13, GDF7, GDF8, GDF9b/BMP15, GDF11/BMP11, GDF15/MIC1, TGF-β1, TGF-β2, TGF-β3, activin A, activin B, activin C, activin E, activin AB, activin AC, activin AE, activin BC, activin BE, nodal, glial cell-derived neurotrophic factor (GDNF), neurturin, artemin, persephin, MIS, and Lefty.

In some embodiments, the present disclosure contemplates making functional variants by modifying the structure of the TGF-beta superfamily type I receptor polypeptide, TGF-beta superfamily type II receptor polypeptide, and/or TGF-beta superfamily co-receptor polypeptide for such purposes as enhancing therapeutic efficacy or stability (e.g., increased shelf-life and/or increased resistance to proteolytic degradation).

In certain embodiments, the present disclosure contemplates specific mutations of a TGF-beta superfamily type I receptor polypeptide (e.g., ALK1, ALK2, ALK3, ALK4, ALK5, ALK6, and ALK7), a TGF-beta superfamily type II receptor polypeptide (e.g., ActRIIA, ActRIIB, TGFBRII, BMPRII, and MISRII), and/or a TGF-beta superfamily co-receptor polypeptide (e.g., endoglin, betaglycan, Cripto-1, Cryptic, Cryptic family protein 1B, CRIM1, CRIM2, BAMBI, BMPER, RGM-A, RGM-B, MuSK, and hemojuvelin) of the disclosure so as to alter the glycosylation of the polypeptide. Such mutations may be selected so as to introduce or eliminate one or more glycosylation sites, such as O-linked or N-linked glycosylation sites. Asparagine-linked glycosylation recognition sites generally comprise a tripeptide sequence, asparagine-X-threonine or asparagine-X-serine (where “X” is any amino acid) which is specifically recognized by appropriate cellular glycosylation enzymes. The alteration may also be made by the addition of, or substitution by, one or more serine or threonine residues to the sequence of the polypeptide (for O-linked glycosylation sites). A variety of amino acid substitutions or deletions at one or both of the first or third amino acid positions of a glycosylation recognition site (and/or amino acid deletion at the second position) results in non-glycosylation at the modified tripeptide sequence. Another means of increasing the number of carbohydrate moieties on a polypeptide is by chemical or enzymatic coupling of glycosides to the polypeptide. Depending on the coupling mode used, the sugar(s) may be attached to (a) arginine and histidine; (b) free carboxyl groups; (c) free sulfhydryl groups such as those of cysteine; (d) free hydroxyl groups such as those of serine, threonine, or hydroxyproline; (e) aromatic residues such as those of phenylalanine, tyrosine, or tryptophan; or (f) the amide group of glutamine. Removal of one or more carbohydrate moieties present on a polypeptide may be accomplished chemically and/or enzymatically. Chemical deglycosylation may involve, for example, exposure of a polypeptide to the compound trifluoromethanesulfonic acid, or an equivalent compound. This treatment results in the cleavage of most or all sugars except the linking sugar (N-acetylglucosamine or N-acetylgalactosamine), while leaving the amino acid sequence intact. Enzymatic cleavage of carbohydrate moieties on polypeptides can be achieved by the use of a variety of endo- and exo-glycosidases as described by Thotakura et al. [Meth. Enzymol. (1987) 138:350]. The sequence of a polypeptide may be adjusted, as appropriate, depending on the type of expression system used, as mammalian, yeast, insect, and plant cells may all introduce differing glycosylation patterns that can be affected by the amino acid sequence of the peptide. In general, heteromultimers of the disclosure for use in humans may be expressed in a mammalian cell line that provides proper glycosylation, such as HEK293 or CHO cell lines, although other mammalian expression cell lines are expected to be useful as well.

The present disclosure further contemplates a method of generating mutants, particularly sets of combinatorial mutants of a TGF-beta superfamily type I receptor polypeptide (e.g., ALK1, ALK2, ALK3, ALK4, ALK5, ALK6, and ALK7), a TGF-beta superfamily type II receptor polypeptide (e.g., ActRIIA, ActRIIB, TGFBRII, BMPRII, and MISRII), and/or TGF-beta superfamily co-receptor polypeptide (e.g., endoglin, betaglycan, Cripto-1, Cryptic, Cryptic family protein 1B, CRIM1, CRIM2, BAMBI, BMPER, RGM-A, RGM-B, MuSK, and hemojuvelin) of the present disclosure, as well as truncation mutants. Pools of combinatorial mutants are especially useful for identifying functionally active (e.g., ligand binding) TGF-beta superfamily type I receptor, TGF-beta superfamily type II receptor, and/or TGF-beta superfamily co-receptor sequences. The purpose of screening such combinatorial libraries may be to generate, for example, polypeptides variants which have altered properties, such as altered pharmacokinetic or altered ligand binding. A variety of screening assays are provided below, and such assays may be used to evaluate variants. For example, TGF-beta co-receptor variants may be screened for ability to bind to a TGF-beta superfamily ligand (e.g., BMP2, BMP2/7, BMP3, BMP4, BMP4/7, BMP5, BMP6, BMP7, BMP8a, BMP8b, BMP9, BMP10, GDF3, GDF5, GDF6/BMP13, GDF7, GDF8, GDF9b/BMP15, GDF11/BMP11, GDF15/MIC1, TGF-β1, TGF-β2, TGF-β3, activin A, activin B, activin C, activin E, activin AB, activin AC, activin AE, activin BC, activin BE, nodal, glial cell-derived neurotrophic factor (GDNF), neurturin, artemin, persephin, MIS, and Lefty), to prevent binding of a TGF-beta superfamily ligand to a TGF-beta superfamily co-receptor, and/or to interfere with signaling caused by an TGF-beta superfamily ligand.

The activity of a TGF-beta superfamily heteromultimers of the disclosure also may be tested, for example in a cell-based or in vivo assay. For example, the effect of a heteromultimer on the expression of genes or the activity of proteins involved in muscle production in a muscle cell may be assessed. This may, as needed, be performed in the presence of one or more recombinant TGF-beta superfamily ligand proteins (e.g., BMP2, BMP2/7, BMP3, BMP4, BMP4/7, BMP5, BMP6, BMP7, BMP8a, BMP8b, BMP9, BMP10, GDF3, GDF5, GDF6/BMP13, GDF7, GDF8, GDF9b/BMP15, GDF11/BMP11, GDF15/MIC1, TGF-β1, TGF-β2, TGF-β3, activin A, activin B, activin C, activin E, activin AB, activin AC, activin AE, activin BC, activin BE, nodal, glial cell-derived neurotrophic factor (GDNF), neurturin, artemin, persephin, MIS, and Lefty), and cells may be transfected so as to produce a TGF-beta superfamily heteromultimer, and optionally, a TGF-beta superfamily ligand. Likewise, a heteromultimer of the disclosure may be administered to a mouse or other animal, and one or more measurements, such as muscle formation and strength may be assessed using art-recognized methods. Similarly, the activity of a heteromultimer, or variants thereof, may be tested in osteoblasts, adipocytes, and/or neuronal cells for any effect on growth of these cells, for example, by the assays as described herein and those of common knowledge in the art. A SMAD-responsive reporter gene may be used in such cell lines to monitor effects on downstream signaling.

Combinatorial-derived variants can be generated which have increased selectivity or generally increased potency relative to a reference TGF-beta superfamily heteromultimer. Such variants, when expressed from recombinant DNA constructs, can be used in gene therapy protocols. Likewise, mutagenesis can give rise to variants which have intracellular half-lives dramatically different than the corresponding unmodified TGF-beta superfamily heteromultimer. For example, the altered protein can be rendered either more stable or less stable to proteolytic degradation or other cellular processes which result in destruction, or otherwise inactivation, of an unmodified polypeptide. Such variants, and the genes which encode them, can be utilized to alter polypeptide complex levels by modulating the half-life of the polypeptide. For instance, a short half-life can give rise to more transient biological effects and, when part of an inducible expression system, can allow tighter control of recombinant polypeptide complex levels within the cell. In an Fc fusion protein, mutations may be made in the linker (if any) and/or the Fc portion to alter one or more activities of the TGF-beta superfamily heteromultimer including, for example, immunogenicity, half-life, and solubility.

A combinatorial library may be produced by way of a degenerate library of genes encoding a library of polypeptides which each include at least a portion of potential TGF-beta superfamily type I receptor polypeptide, type II receptor polypeptide, and/or co-receptor polypeptide sequences. For instance, a mixture of synthetic oligonucleotides can be enzymatically ligated into gene sequences such that the degenerate set of potential TGF-beta superfamily type I receptor polypeptide, type II receptor polypeptide, and/or co-receptor encoding nucleotide sequences are expressible as individual polypeptides, or alternatively, as a set of larger fusion proteins (e.g., for phage display).

There are many ways by which the library of potential homologs can be generated from a degenerate oligonucleotide sequence. Chemical synthesis of a degenerate gene sequence can be carried out in an automatic DNA synthesizer, and the synthetic genes can then be ligated into an appropriate vector for expression. The synthesis of degenerate oligonucleotides is well known in the art. See, e.g., Narang, S A (1983) Tetrahedron 39:3; Itakura et al. (1981) Recombinant DNA, Proc. 3rd Cleveland Sympos. Macromolecules, ed. AG Walton, Amsterdam: Elsevier pp 273-289; Itakura et al. (1984) Annu. Rev. Biochem. 53:323; Itakura et al. (1984) Science 198:1056; Ike et al. (1983) Nucleic Acid Res. 11:477. Such techniques have been employed in the directed evolution of other proteins. See, e.g., Scott et al., (1990) Science 249:386-390; Roberts et al. (1992) PNAS USA 89:2429-2433; Devlin et al. (1990) Science 249: 404-406; Cwirla et al., (1990) PNAS USA 87: 6378-6382; as well as U.S. Pat. Nos. 5,223,409, 5,198,346, and 5,096,815.

Alternatively, other forms of mutagenesis can be utilized to generate a combinatorial library. For example, heteromultimers of the disclosure can be generated and isolated from a library by screening using, for example, alanine scanning mutagenesis [see, e.g., Ruf et al. (1994) Biochemistry 33:1565-1572; Wang et al. (1994) J. Biol. Chem. 269:3095-3099; Balint et al. (1993) Gene 137:109-118; Grodberg et al. (1993) Eur. J. Biochem. 218:597-601; Nagashima et al. (1993) J. Biol. Chem. 268:2888-2892; Lowman et al. (1991) Biochemistry 30:10832-10838; and Cunningham et al. (1989) Science 244:1081-1085], by linker scanning mutagenesis [see, e.g., Gustin et al. (1993) Virology 193:653-660; and Brown et al. (1992) Mol. Cell Biol. 12:2644-2652; McKnight et al. (1982) Science 232:316], by saturation mutagenesis [see, e.g., Meyers et al., (1986) Science 232:613]; by PCR mutagenesis [see, e.g., Leung et al. (1989) Method Cell Mol Biol 1:11-19]; or by random mutagenesis, including chemical mutagenesis [see, e.g., Miller et al. (1992) A Short Course in Bacterial Genetics, CSHL Press, Cold Spring Harbor, N.Y.; and Greener et al. (1994) Strategies in Mol Biol 7:32-34]. Linker scanning mutagenesis, particularly in a combinatorial setting, is an attractive method for identifying truncated (bioactive) forms of TGF-beta superfamily type I receptor, type II receptor, and/or co-receptor polypeptides.

A wide range of techniques are known in the art for screening gene products of combinatorial libraries made by point mutations and truncations, and, for that matter, for screening cDNA libraries for gene products having a certain property. Such techniques will be generally adaptable for rapid screening of the gene libraries generated by the combinatorial mutagenesis of heteromultimers of the disclosure. The most widely used techniques for screening large gene libraries typically comprise cloning the gene library into replicable expression vectors, transforming appropriate cells with the resulting library of vectors, and expressing the combinatorial genes under conditions in which detection of a desired activity facilitates relatively easy isolation of the vector encoding the gene whose product was detected. Preferred assays include TGF-beta superfamily ligand (e.g., BMP2, BMP2/7, BMP3, BMP4, BMP4/7, BMP5, BMP6, BMP7, BMP8a, BMP8b, BMP9, BMP10, GDF3, GDF5, GDF6/BMP13, GDF7, GDF8, GDF9b/BMP15, GDF11/BMP11, GDF15/MIC1, TGF-β1, TGF-β2, TGF-β3, activin A, activin B, activin C, activin E, activin AB, activin AC, activin AE, activin BC, activin BE, nodal, glial cell-derived neurotrophic factor (GDNF), neurturin, artemin, persephin, MIS, and Lefty) binding assays and/or TGF-beta superfamily ligand-mediated cell signaling assays.

In certain embodiments, heteromultimers of the disclosure may further comprise post-translational modifications in addition to any that are naturally present in the TGF-beta superfamily type I receptor, type II receptor, or co-receptor polypeptide. Such modifications include, but are not limited to, acetylation, carboxylation, glycosylation, phosphorylation, lipidation, and acylation. As a result, the heteromultimers may comprise non-amino acid elements, such as polyethylene glycols, lipids, polysaccharide or monosaccharide, and phosphates. Effects of such non-amino acid elements on the functionality of a heteromultimer may be tested as described herein for other heteromultimer variants. When a polypeptide of the disclosure is produced in cells by cleaving a nascent form of the polypeptide, post-translational processing may also be important for correct folding and/or function of the protein. Different cells (e.g., CHO, HeLa, MDCK, 293, WI38, NIH-3T3 or HEK293) have specific cellular machinery and characteristic mechanisms for such post-translational activities and may be chosen to ensure the correct modification and processing of the TGF-beta superfamily type I receptor, type II receptor, and/or co-receptor polypeptides as well as heteromultimers comprising the same.

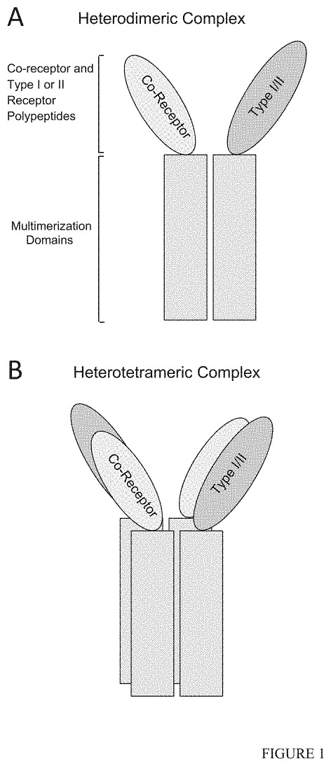

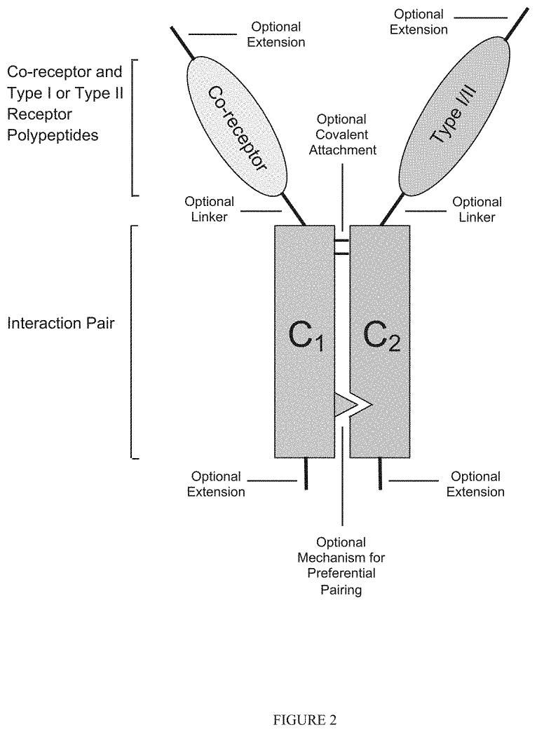

In certain aspects, the polypeptides disclosed herein may form heteromultimers comprising at least one TGF-beta superfamily co-receptor polypeptide associated, covalently or non-covalently, with at least one type I receptor polypeptide, type I receptor polypeptide, or another co-receptor polypeptide. Preferably, polypeptides disclosed herein form heterodimers, although higher order heteromultimers are also included such as, but not limited to, heterotrimers, heterotetramers, and further oligomeric structures (see, e.g., FIGS. 1, 2, and 6-10). In some embodiments, TGF-beta superfamily type I receptor, type II receptor, and/or co-receptor polypeptides of the present disclosure comprise at least one multimerization domain. As disclosed herein, the term “multimerization domain” refers to an amino acid or sequence of amino acids that promote covalent or non-covalent interaction between at least a first polypeptide and at least a second polypeptide. Polypeptides disclosed herein may be joined covalently or non-covalently to a multimerization domain. Preferably, a multimerization domain promotes interaction between a first polypeptide (e.g., TGF-beta superfamily co-receptor polypeptide) and a second polypeptide (e.g., TGF-beta superfamily type I or II receptor polypeptide) to promote heteromultimer formation (e.g., heterodimer formation), and optionally hinders or otherwise disfavors homomultimer formation (e.g., homodimer formation), thereby increasing the yield of desired heteromultimer (see, e.g., FIG. 2).

Many methods known in the art can be used to generate heteromultimers of the disclosure. For example, non-naturally occurring disulfide bonds may be constructed by replacing on a first polypeptide (e.g., TGF-beta superfamily co-receptor polypeptide) a naturally occurring amino acid with a free thiol-containing residue, such as cysteine, such that the free thiol interacts with another free thiol-containing residue on a second polypeptide (e.g., TGF-beta superfamily type I or type II receptor polypeptide) such that a disulfide bond is formed between the first and second polypeptides. Additional examples of interactions to promote heteromultimer formation include, but are not limited to, ionic interactions such as described in Kjaergaard et al., WO2007147901; electrostatic steering effects such as described in Kannan et al., U.S. Pat. No. 8,592,562; coiled-coil interactions such as described in Christensen et al., U.S.20120302737; leucine zippers such as described in Pack & Plueckthun, (1992) Biochemistry 31: 1579-1584; and helix-turn-helix motifs such as described in Pack et al., (1993) Bio/Technology 11: 1271-1277. Linkage of the various segments may be obtained via, e.g., covalent binding such as by chemical cross-linking, peptide linkers, disulfide bridges, etc., or affinity interactions such as by avidin-biotin or leucine zipper technology.

In certain aspects, a multimerization domain may comprise one component of an interaction pair. In some embodiments, the polypeptides disclosed herein may form protein complexes comprising a first polypeptide covalently or non-covalently associated with a second polypeptide, wherein the first polypeptide comprises the amino acid sequence of a TGF-beta superfamily co-receptor polypeptide and the amino acid sequence of a first member of an interaction pair; and the second polypeptide comprises the amino acid sequence of a TGF-beta superfamily type I receptor, type II receptor, or another co-receptor polypeptide and the amino acid sequence of a second member of an interaction pair. The interaction pair may be any two polypeptide sequences that interact to form a complex, particularly a heterodimeric complex although operative embodiments may also employ an interaction pair that can form a homodimeric complex. One member of the interaction pair may be fused to a TGF-beta superfamily type I receptor, type II receptor, or co-receptor polypeptide as described herein, including for example, a polypeptide sequence comprising an amino acid sequence that is at least 70%, 75%, 80%, 85%, 90%, 91%, 92%, 93%, 94%, 95%, 96%, 97%, 98%, 99%, or 100% identical to the sequence of any one of SEQ ID NOs: 1, 2, 3, 4, 5, 6, 9, 10, 11, 14, 15, 18, 19, 22, 23, 26, 27, 30, 31, 34, 35, 38, 39, 42, 43, 46, 47, 50, 51, 67, 68, 71, 72, 75, 76, 79, 80, 83, 84, 87, 88, 91, 92, 301, 302, 305, 306, 309, 310, 313, 501, 502, 505, 506, 509, 510, 513, 514, 517, 518, 521, 522, 525, 526, 529, 530, 533, 534, 537, 538, 541, 542, 545, 546, 549, 550, 553, 554, 557, 558, 561, 562, 565, 566, 569, 570, 573, 574, 577, 578, 581, 582, 585, 586, 589, 590, 593, 594, 595, 596, 599, 600, 603, and 604. An interaction pair may be selected to confer an improved property/activity such as increased serum half-life, or to act as an adaptor on to which another moiety is attached to provide an improved property/activity. For example, a polyethylene glycol moiety may be attached to one or both components of an interaction pair to provide an improved property/activity such as improved serum half-life.

The first and second members of the interaction pair may be an asymmetric pair, meaning that the members of the pair preferentially associate with each other rather than self-associate. Accordingly, first and second members of an asymmetric interaction pair may associate to form a heterodimeric complex (see, e.g., FIG. 2). Alternatively, the interaction pair may be unguided, meaning that the members of the pair may associate with each other or self-associate without substantial preference and thus may have the same or different amino acid sequences. Accordingly, first and second members of an unguided interaction pair may associate to form a homodimer complex or a heterodimeric complex. Optionally, the first member of the interaction pair (e.g., an asymmetric pair or an unguided interaction pair) associates covalently with the second member of the interaction pair. Optionally, the first member of the interaction pair (e.g., an asymmetric pair or an unguided interaction pair) associates non-covalently with the second member of the interaction pair.

As specific examples, the present disclosure provides fusion proteins comprising TGF-beta superfamily type I receptor, type II receptor, or co-receptor polypeptides fused to a polypeptide comprising a constant domain of an immunoglobulin, such as a CH1, CH2, or CH3 domain of an immunoglobulin or an Fc domain. Fc domains derived from human IgG1, IgG2, IgG3, and IgG4 are provided herein. Other mutations are known that decrease either CDC or ADCC activity, and collectively, any of these variants are included in the disclosure and may be used as advantageous components of a heteromultimers of the disclosure. Optionally, the IgG1 Fc domain of SEQ ID NO: 208 has one or more mutations at residues such as Asp-265, Lys-322, and Asn-434 (numbered in accordance with the corresponding full-length IgG1). In certain cases, the mutant Fc domain having one or more of these mutations (e.g., Asp-265 mutation) has reduced ability of binding to the Fcγ receptor relative to a wildtype Fc domain. In other cases, the mutant Fc domain having one or more of these mutations (e.g., Asn-434 mutation) has increased ability of binding to the MHC class I-related Fc-receptor (FcRN) relative to a wildtype Fc domain.

An example of a native amino acid sequence that may be used for the Fc portion of human IgG1 (G1Fc) is shown below (SEQ ID NO: 208). Dotted underline indicates the hinge region, and solid underline indicates positions with naturally occurring variants. In part, the disclosure provides polypeptides comprising, consisting of, or consisting essentially of an amino acid sequence with 70%, 80%, 85%, 86%, 87%, 88%, 89%, 90%, 91%, 92%, 93%, 94%, 95%, 96%, 97%, 98%, 99%, or 100% identity to SEQ ID NO: 208. Naturally occurring variants in G1Fc would include E134D and M136L according to the numbering system used in SEQ ID NO: 208 (see Uniprot P01857).

| (SEQ ID NO: 208) | |
|---|---|
| 1 | 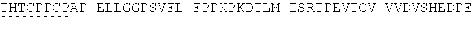 |
| 51 | VKFNWYVDGV EVHNAKTKPR EEQYNSTYRV VSVLTVLHQD WLNGKEYKCK |
| 101 | VSNKALPAPI EKTISKAKGQ PREPQVYTLP PSREEMTKNQ VSLTCLVKGF |
| 151 | YPSDIAVEWE SNGQPENNYK TTPPVLDSDG SFFLYSKLTV DKSRWQQGNV |
| 201 | FSCSVMHEAL HNHYTQKSLS LSPGK |

An example of a native amino acid sequence that may be used for the Fc portion of human IgG2 (G2Fc) is shown below (SEQ ID NO: 209). Dotted underline indicates the hinge region and double underline indicates positions where there are data base conflicts in the sequence (according to UniProt P01859). In part, the disclosure provides polypeptides comprising, consisting of, or consisting essentially of an amino acid sequence with 70%, 80%, 85%, 86%, 87%, 88%, 89%, 90%, 91%, 92%, 93%, 94%, 95%, 96%, 97%, 98%, 99%, or 100% identity to SEQ ID NO: 209.

| (SEQ ID NO: 209) | |
|---|---|
| 1 | 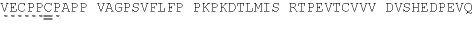 |
| 51 | FNWYVDGVEV HNAKTKPREE QFNSTFRVVS VLTVVHQDWL NGKEYKCKVS |
| 101 | NKGLPAPIEK TISKTKGQPR EPQVYTLPPS REEMTKNQVS LTCLVKGFYP |
| 151 | SDIAVEWESN GQPENNYKTT PPMLDSDGSF FLYSKLTVDK SRWQQGNVFS |
| 201 | CSVMHEALHN HYTQKSLSLS PGK |

Two examples of amino acid sequences that may be used for the Fc portion of human IgG3 (G3Fc) are shown below. The hinge region in G3Fc can be up to four times as long as in other Fc chains and contains three identical 15-residue segments preceded by a similar 17-residue segment. The first G3Fc sequence shown below (SEQ ID NO: 210) contains a short hinge region consisting of a single 15-residue segment, whereas the second G3Fc sequence (SEQ ID NO: 211) contains a full-length hinge region. In each case, dotted underline indicates the hinge region, and solid underline indicates positions with naturally occurring variants according to UniProt P01859. In part, the disclosure provides polypeptides comprising, consisting of, or consisting essentially of an amino acid sequence with 70%, 80%, 85%, 86%, 87%, 88%, 89%, 90%, 91%, 92%, 93%, 94%, 95%, 96%, 97%, 98%, 99%, or 100% identity to SEQ ID NOs: 210 and 211.

| (SEQ ID NO: 210) | |
|---|---|
| 1 | 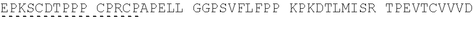 |
| 51 | VSHEDPEVQF KWYVDGVEVH NAKTKPREEQ YNSTFRVVSV LTVLHQDWLN |
| 101 | GKEYKCKVSN KALPAPIEKT ISKTKGQPRE PQVYTLPPSR EEMTKNQVSL |
| 151 | TCLVKGFYPS DIAVEWESSG QPENNYNTTP PMLDSDGSFF LYSKLTVDKS |
| 201 | RWQQGNIFSC SVMHEALHNR FTQKSLSLSP GK |
| (SEQ ID NO: 211) | |
| 1 | 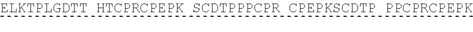 |
| 51 | 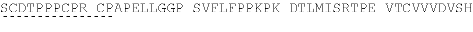 |
| 101 | EDPEVQFKWY VDGVEVHNAK TKPREEQYNS TFRVVSVLTV LHQDWLNGKE |
| 151 | YKCKVSNKAL PAPIEKTISK TKGQPREPQV YTLPPSREEM TKNQVSLTCL |
| 201 | VKGFYPSDIA VEWESSGQPE NNYNTTPPML DSDGSFFLYS KLTVDKSRWQ |
| 251 | QGNIFSCSVM HEALHNRFTQ KSLSLSPGK |

Naturally occurring variants in G3Fc (for example, see Uniprot P01860) include E68Q, P76L, E79Q, Y81F, D97N, N100D, T124A, S169N, S169del, F221Y when converted to the numbering system used in SEQ ID NO: 210, and the present disclosure provides fusion proteins comprising G3Fc domains containing one or more of these variations. In addition, the human immunoglobulin IgG3 gene (IGHG3) shows a structural polymorphism characterized by different hinge lengths [see Uniprot P01859]. Specifically, variant WIS is lacking most of the V region and all of the CH1 region. It has an extra interchain disulfide bond at position 7 in addition to the 11 normally present in the hinge region. Variant ZUC lacks most of the V region, all of the CH1 region, and part of the hinge. Variant OMM may represent an allelic form or another gamma chain subclass. The present disclosure provides additional fusion proteins comprising G3Fc domains containing one or more of these variants.

An example of a native amino acid sequence that may be used for the Fc portion of human IgG4 (G4Fc) is shown below (SEQ ID NO: 212). Dotted underline indicates the hinge region. In part, the disclosure provides polypeptides comprising, consisting of, or consisting essentially of an amino acid sequence with 70%, 80%, 85%, 86%, 87%, 88%, 89%, 90%, 91%, 92%, 93%, 94%, 95%, 96%, 97%, 98%, 99%, or 100% identity to SEQ ID NO: 212.

| (SEQ ID NO: 212) | |
|---|---|
| 1 | 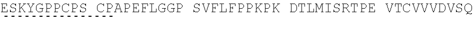 |
| 51 | EDPEVQFNWY VDGVEVHNAK TKPREEQFNS TYRVVSVLTV LHQDWLNGKE |
| 101 | YKCKVSNKGL PSSIEKTISK AKGQPREPQV YTLPPSQEEM TKNQVSLTCL |
| 151 | VKGFYPSDIA VEWESNGQPE NNYKTTPPVL DSDGSFFLYS RLTVDKSRWQ |
| 201 | EGNVFSCSVM HEALHNHYTQ KSLSLSLGK |

A variety of engineered mutations in the Fc domain are presented herein with respect to the G1Fc sequence (SEQ ID NO: 208), and analogous mutations in G2Fc, G3Fc, and G4Fc can be derived from their alignment with G1Fc in FIG. 5. Due to unequal hinge lengths, analogous Fc positions based on isotype alignment (FIG. 5) possess different amino acid numbers in SEQ ID NOs: 208, 209, 210, and 212. It can also be appreciated that a given amino acid position in an immunoglobulin sequence consisting of hinge, C_{H}2, and C_{H}3 regions (e.g., SEQ ID NOs: 208, 209, 210, 211, or 212) will be identified by a different number than the same position when numbering encompasses the entire IgG1 heavy-chain constant domain (consisting of the C_{H}1, hinge, C_{H}2, and C_{H}3 regions) as in the Uniprot database. For example, correspondence between selected C_{H}3 positions in a human G1Fc sequence (SEQ ID NO: 208), the human IgG1 heavy chain constant domain (Uniprot P01857), and the human IgG1 heavy chain is as follows.

Correspondence of C_{H}3 Positions in Different Numbering Systems

| G1Fc | IgG1 heavy chain | IgG1 heavy chain |
|---|---|---|
| (Numbering begins | constant domain | (EU numbering |
| at first threonine | (Numbering | scheme of |
| in hinge region) | begins at C_{H}1) | Kabat et al., 1991*) |
| Y127 | Y232 | Y349 |
| S132 | S237 | S354 |
| E134 | E239 | E356 |
| K138 | K243 | K360 |
| T144 | T249 | T366 |
| L146 | L251 | L368 |
| N162 | N267 | N384 |
| K170 | K275 | K392 |
| D177 | D282 | D399 |
| D179 | D284 | D401 |
| Y185 | Y290 | Y407 |
| K187 | K292 | K409 |
| H213 | H318 | H435 |
| K217 | K322 | K439 |

*Kabat et al. (eds) 1991; pp. 688-696 in Sequences of Proteins of Immunological Interest, 5^{th} ed., Vol. 1, NIH, Bethesda, MD.

A problem that arises in large-scale production of asymmetric immunoglobulin-based proteins from a single cell line is known as the “chain association issue”. As confronted prominently in the production of bispecific antibodies, the chain-association issue concerns the challenge of efficiently producing a desired multichain protein from among the multiple combinations that inherently result when different heavy chains and/or light chains are produced in a single cell line [see, for example, Klein et al (2012) mAbs 4:653-663]. This problem is most acute when two different heavy chains and two different light chains are produced in the same cell, in which case there are a total of 16 possible chain combinations (although some of these are identical) when only one is typically desired. Nevertheless, the same principle accounts for diminished yield of a desired multichain fusion protein that incorporates only two different (asymmetric) heavy chains.

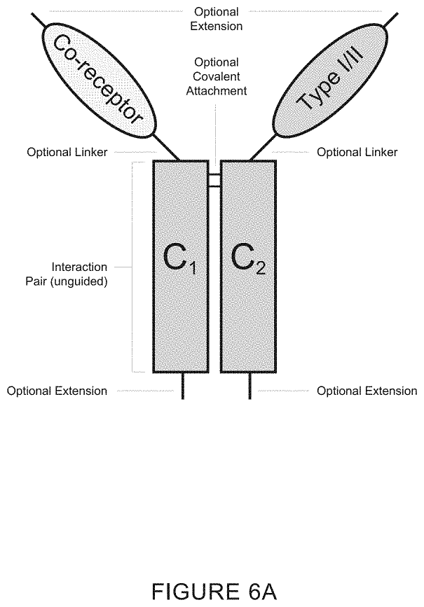

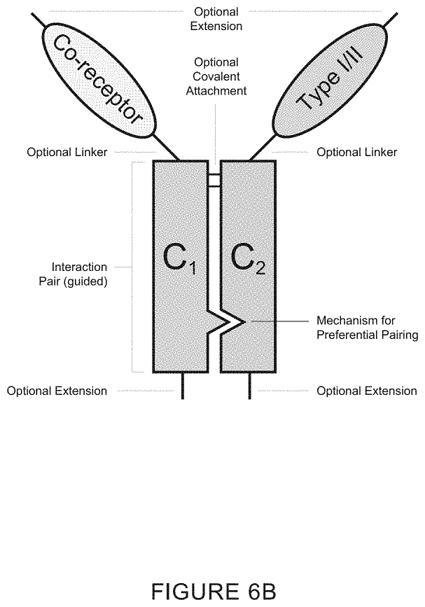

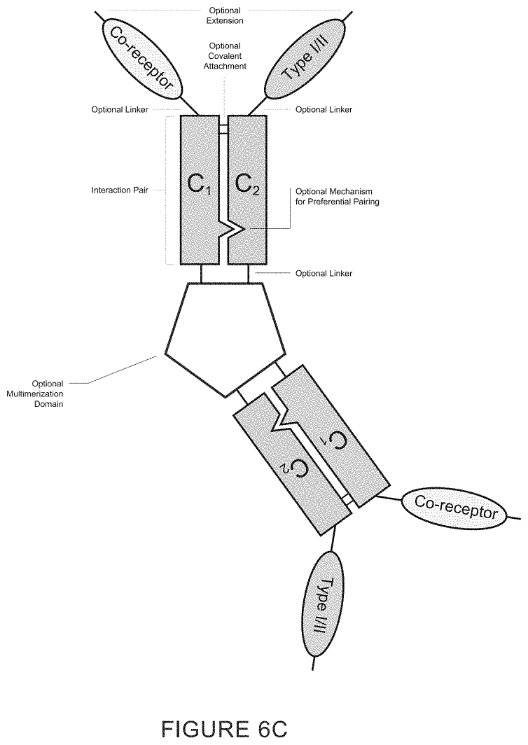

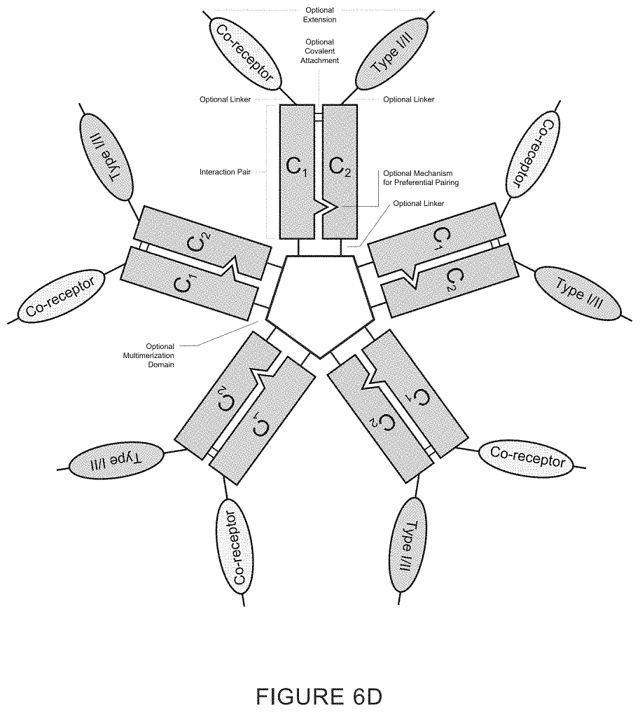

Various methods are known in the art that increase desired pairing of Fc-containing fusion polypeptide chains in a single cell line to produce a preferred asymmetric fusion protein at acceptable yields [see, for example, Klein et al (2012) mAbs 4:653-663; and Spiess et al (2015) Molecular Immunology 67(2A): 95-106]. Methods to obtain desired pairing of Fc-containing chains include, but are not limited to, charge-based pairing (electrostatic steering), “knobs-into-holes” steric pairing, SEEDbody pairing, and leucine zipper-based pairing. See, for example, Ridgway et al (1996) Protein Eng 9:617-621; Merchant et al (1998) Nat Biotech 16:677-681; Davis et al (2010) Protein Eng Des Sel 23:195-202; Gunasekaran et al (2010); 285:19637-19646; Wranik et al (2012) J Biol Chem 287:43331-43339; U.S. Pat. No. 5,932,448; WO 1993/011162; WO 2009/089004, and WO 2011/034605. As described herein, these methods may be used to generate heterodimers comprising a TGF-beta superfamily co-receptor and a TGF-beta type I or type II receptor polypeptide or another, optionally different, TGF-beta superfamily co-receptor polypeptide. See FIGS. 6-10.

For example, one means by which interaction between specific polypeptides may be promoted is by engineering protuberance-into-cavity (knob-into-holes) complementary regions such as described in Arathoon et al., U.S. Pat. No. 7,183,076 and Carter et al., U.S. Pat. No. 5,731,168. “Protuberances” are constructed by replacing small amino acid side chains from the interface of the first polypeptide (e.g., a first interaction pair) with larger side chains (e.g., tyrosine or tryptophan). Complementary “cavities” of identical or similar size to the protuberances are optionally created on the interface of the second polypeptide (e.g., a second interaction pair) by replacing large amino acid side chains with smaller ones (e.g., alanine or threonine). Where a suitably positioned and dimensioned protuberance or cavity exists at the interface of either the first or second polypeptide, it is only necessary to engineer a corresponding cavity or protuberance, respectively, at the adjacent interface.

At neutral pH (7.0), aspartic acid and glutamic acid are negatively charged and lysine, arginine, and histidine are positively charged. These charged residues can be used to promote heterodimer formation and at the same time hinder homodimer formation. Attractive interactions take place between opposite charges and repulsive interactions occur between like charges. In part, protein complexes disclosed herein make use of the attractive interactions for promoting heteromultimer formation (e.g., heterodimer formation), and optionally repulsive interactions for hindering homodimer formation (e.g., homodimer formation) by carrying out site directed mutagenesis of charged interface residues.

For example, the IgG1 CH3 domain interface comprises four unique charge residue pairs involved in domain-domain interactions: Asp356-Lys439′, Glu357-Lys370′, Lys392-Asp399′, and Asp399-Lys409′ [residue numbering in the second chain is indicated by (′)]. It should be noted that the numbering scheme used here to designate residues in the IgG1 CH3 domain conforms to the EU numbering scheme of Kabat. Due to the 2-fold symmetry present in the CH3-CH3 domain interactions, each unique interaction will represented twice in the structure (e.g., Asp-399-Lys409′ and Lys409-Asp399′). In the wild-type sequence, K409-D399′ favors both heterodimer and homodimer formation. A single mutation switching the charge polarity (e.g., K409E; positive to negative charge) in the first chain leads to unfavorable interactions for the formation of the first chain homodimer. The unfavorable interactions arise due to the repulsive interactions occurring between the same charges (negative-negative; K409E-D399′ and D399-K409E′). A similar mutation switching the charge polarity (D399K′; negative to positive) in the second chain leads to unfavorable interactions (K409′-D399K′ and D399K-K409′) for the second chain homodimer formation. But, at the same time, these two mutations (K409E and D399K′) lead to favorable interactions (K409E-D399K′ and D399-K409′) for the heterodimer formation.

The electrostatic steering effect on heterodimer formation and homodimer discouragement can be further enhanced by mutation of additional charge residues which may or may not be paired with an oppositely charged residue in the second chain including, for example, Arg355 and Lys360. The table below lists possible charge change mutations that can be used, alone or in combination, to enhance heteromultimer formation of the heteromultimers disclosed herein.

Examples of Pair-Wise Charged Residue
Mutations to Enhance Heterodimer Formation

| | | Interacting | Corresponding |
|---|---|---|---|
| Position in | Mutation in | position in | mutation in |
| first chain | first chain | second chain | second chain |
| Lys409 | Asp or Glu | Asp399' | Lys, Arg, or His |
| Lys392 | Asp or Glu | Asp399' | Lys, Arg, or His |
| Lys439 | Asp or Glu | Asp356' | Lys, Arg, or His |
| Lys370 | Asp or Glu | Glu357' | Lys, Arg, or His |
| Asp399 | Lys, Arg, or His | Lys409' | Asp or Glu |
| Asp399 | Lys, Arg, or His | Lys392' | Asp or Glu |
| Asp356 | Lys, Arg, or His | Lys439' | Asp or Glu |
| Glu357 | Lys, Arg, or His | Lys370' | Asp or Glu |

In some embodiments, one or more residues that make up the CH3-CH3 interface in a fusion protein of the instant application are replaced with a charged amino acid such that the interaction becomes electrostatically unfavorable. For example, a positive-charged amino acid in the interface (e.g., a lysine, arginine, or histidine) is replaced with a negatively charged amino acid (e.g., aspartic acid or glutamic acid). Alternatively, or in combination with the forgoing substitution, a negative-charged amino acid in the interface is replaced with a positive-charged amino acid. In certain embodiments, the amino acid is replaced with a non-naturally occurring amino acid having the desired charge characteristic. It should be noted that mutating negatively charged residues (Asp or Glu) to His will lead to increase in side chain volume, which may cause steric issues. Furthermore, His proton donor- and acceptor-form depends on the localized environment. These issues should be taken into consideration with the design strategy. Because the interface residues are highly conserved in human and mouse IgG subclasses, electrostatic steering effects disclosed herein can be applied to human and mouse IgG1, IgG2, IgG3, and IgG4. This strategy can also be extended to modifying uncharged residues to charged residues at the CH3 domain interface.

In part, the disclosure provides desired pairing of asymmetric Fc-containing polypeptide chains using Fc sequences engineered to be complementary on the basis of charge pairing (electrostatic steering). One of a pair of Fc sequences with electrostatic complementarity can be arbitrarily fused to the TGF-beta superfamily type I receptor polypeptide, type II receptor polypeptide, or co-receptor polypeptide of the construct, with or without an optional linker, to generate a TGF-beta superfamily type I, type II, or co-receptor receptor fusion polypeptide This single chain can be coexpressed in a cell of choice along with the Fc sequence complementary to the first Fc to favor generation of the desired multichain construct (e.g., a TGF-beta superfamily heteromultimer). In this example based on electrostatic steering, SEQ ID NO: 200 [human G1Fc(E134K/D177K)] and SEQ ID NO: 201 [human G1Fc(K170D/K187D)] are examples of complementary Fc sequences in which the engineered amino acid substitutions are double underlined, and the TGF-beta superfamily type I/II receptor polypeptide or co-receptor polypeptide of the construct can be fused to either SEQ ID NO: 200 or SEQ ID NO: 201, but not both. Given the high degree of amino acid sequence identity between native hG1Fc, native hG2Fc, native hG3Fc, and native hG4Fc, it can be appreciated that amino acid substitutions at corresponding positions in hG2Fc, hG3Fc, or hG4Fc (see FIG. 5) will generate complementary Fc pairs which may be used instead of the complementary hG1Fc pair below (SEQ ID NOs: 200 and 201).

| (SEQ ID NO: 200) | |
|---|---|
| 1 | THTCPPCPAP ELLGGPSVFL FPPKPKDTLM ISRTPEVTCV VVDVSHEDPE |
| 51 | VKFNWYVDGV EVHNAKTKPR EEQYNSTYRV VSVLTVLHQD WLNGKEYKCK |
| 101 | VSNKALPAPI EKTISKAKGQ PREPQVYTLP PSRKEMTKNQ VSLTCLVKGF |
| 151 | YPSDIAVEWE SNGQPENNYK TTPPVLKSDG SFFLYSKLTV DKSRWQQGNV |
| 201 | FSCSVMHEAL HNHYTQKSLS LSPGK |
| (SEQ ID NO: 201) | |
| 1 | THTCPPCPAP ELLGGPSVFL FPPKPKDTLM ISRTPEVTCV VVDVSHEDPE |
| 51 | VKFNWYVDGV EVHNAKTKPR EEQYNSTYRV VSVLTVLHQD WLNGKEYKCK |
| 101 | VSNKALPAPI EKTISKAKGQ PREPQVYTLP PSREEMTKNQ VSLTCLVKGF |
| 151 | YPSDIAVEWE SNGQPENNYD TTPPVLDSDG SFFLYSDLTV DKSRWQQGNV |
| 201 | FSCSVMHEAL HNHYTQKSLS LSPGK |

In part, the disclosure provides desired pairing of asymmetric Fc-containing polypeptide chains using Fc sequences engineered for steric complementarity. In part, the disclosure provides knobs-into-holes pairing as an example of steric complementarity. One of a pair of Fc sequences with steric complementarity can be arbitrarily fused to the TGF-beta superfamily type I receptor polypeptide, type II receptor polypeptide, or co-receptor polypeptide of the construct, with or without an optional linker, to generate a TGF-beta superfamily type I, type II, or co-receptor fusion polypeptide. This single chain can be coexpressed in a cell of choice along with the Fc sequence complementary to the first Fc to favor generation of the desired multichain construct. In this example based on knobs-into-holes pairing, SEQ ID NO: 202 [human G1Fc(T144Y)] and SEQ ID NO: 203 [human G1Fc(Y185T)] are examples of complementary Fc sequences in which the engineered amino acid substitutions are double underlined, and the TGF-beta superfamily type I receptor polypeptide, type II receptor polypeptide, or co-receptor polypeptide of the construct can be fused to either SEQ ID NO: 202 or SEQ ID NO: 203, but not both. Given the high degree of amino acid sequence identity between native hG1Fc, native hG2Fc, native hG3Fc, and native hG4Fc, it can be appreciated that amino acid substitutions at corresponding positions in hG2Fc, hG3Fc, or hG4Fc (see FIG. 5) will generate complementary Fc pairs which may be used instead of the complementary hG1Fc pair below (SEQ ID NOs: 202 and 203).

| (SEQ ID NO: 202) | |
|---|---|
| 1 | THTCPPCPAP ELLGGPSVFL FPPKPKDTLM ISRTPEVTCV VVDVSHEDPE |
| 51 | VKFNWYVDGV EVHNAKTKPR EEQYNSTYRV VSVLTVLHQD WLNGKEYKCK |
| 101 | VSNKALPAPI EKTISKAKGQ PREPQVYTLP PSREEMTKNQ VSLYCLVKGF |
| 151 | YPSDIAVEWE SNGQPENNYK TTPPVLDSDG SFFLYSKLTV DKSRWQQGNV |
| 201 | FSCSVMHEAL HNHYTQKSLS LSPGK |
| (SEQ ID NO: 203) | |
| 1 | THTCPPCPAP ELLGGPSVFL FPPKPKDTLM ISRTPEVTCV VVDVSHEDPE |
| 51 | VKFNWYVDGV EVHNAKTKPR EEQYNSTYRV VSVLTVLHQD WLNGKEYKCK |
| 101 | VSNKALPAPI EKTISKAKGQ PREPQVYTLP PSREEMTKNQ VSLTCLVKGF |
| 151 | YPSDIAVEWE SNGQPENNYK TTPPVLDSDG SFFLTSKLTV DKSRWQQGNV |
| 201 | FSCSVMHEAL HNHYTQKSLS LSPGK |

An example of Fc complementarity based on knobs-into-holes pairing combined with an engineered disulfide bond is disclosed in SEQ ID NO: 204 [hG1Fc(S132C/T144W)] and SEQ ID NO: 205 [hG1Fc(Y127C/T144S/L146A/Y185V)]. The engineered amino acid substitutions in these sequences are double underlined, and the TGF-beta superfamily type I/II receptor polypeptide or co-receptor of the construct can be fused to either SEQ ID NO: 204 or SEQ ID NO: 205, but not both. Given the high degree of amino acid sequence identity between native hG1Fc, native hG2Fc, native hG3Fc, and native hG4Fc, it can be appreciated that amino acid substitutions at corresponding positions in hG2Fc, hG3Fc, or hG4Fc (see FIG. 5) will generate complementary Fc pairs which may be used instead of the complementary hG1Fc pair below (SEQ ID NOs: 204 and 205).

| (SEQ ID NO: 204) | |
|---|---|
| 1 | THTCPPCPAP ELLGGPSVFL FPPKPKDTLM ISRTPEVTCV VVDVSHEDPE |
| 51 | VKFNWYVDGV EVHNAKTKPR EEQYNSTYRV VSVLTVLHQD WLNGKEYKCK |
| 101 | VSNKALPAPI EKTISKAKGQ PREPQVYTLP PCREEMTKNQ VSLWCLVKGF |
| 151 | YPSDIAVEWE SNGQPENNYK TTPPVLDSDG SFFLYSKLTV DKSRWQQGNV |
| 201 | FSCSVMHEAL HNHYTQKSLS LSPGK |
| (SEQ ID NO: 205) | |
| 1 | THTCPPCPAP ELLGGPSVFL FPPKPKDTLM ISRTPEVTCV VVDVSHEDPE |
| 51 | VKFNWYVDGV EVHNAKTKPR EEQYNSTYRV VSVLTVLHQD WLNGKEYKCK |
| 101 | VSNKALPAPI EKTISKAKGQ PREPQVCTLP PSREEMTKNQ VSLSCAVKGF |
| 151 | YPSDIAVEWE SNGQPENNYK TTPPVLDSDG SFFLVSKLTV DKSRWQQGNV |
| 201 | FSCSVMHEAL HNHYTQKSLS LSPGK |

In part, the disclosure provides desired pairing of asymmetric Fc-containing polypeptide chains using Fc sequences engineered to generate interdigitating β-strand segments of human IgG and IgA C_{H}3 domains. Such methods include the use of strand-exchange engineered domain (SEED) C_{H}3 heterodimers allowing the formation of SEEDbody fusion proteins [see, for example, Davis et al (2010) Protein Eng Design Sel 23:195-202]. One of a pair of Fc sequences with SEEDbody complementarity can be arbitrarily fused to the TGF-beta superfamily type I receptor polypeptide, type II receptor polypeptide or co-receptor polypeptide of the construct, with or without an optional linker, to generate a TGF-beta superfamily fusion polypeptide. This single chain can be coexpressed in a cell of choice along with the Fc sequence complementary to the first Fc to favor generation of the desired multichain construct. In this example based on SEEDbody (Sb) pairing, SEQ ID NO: 206 [hG1Fc(Sb_{AG})] and SEQ ID NO: 207 [hG1Fc(Sb_{GA})] are examples of complementary IgG Fc sequences in which the engineered amino acid substitutions from IgA Fc are double underlined, and the TGF-beta superfamily type I or type II polypeptide of the construct can be fused to either SEQ ID NO: 206 or SEQ ID NO: 207, but not both. Given the high degree of amino acid sequence identity between native hG1Fc, native hG2Fc, native hG3Fc, and native hG4Fc, it can be appreciated that amino acid substitutions at corresponding positions in hG1Fc, hG2Fc, hG3Fc, or hG4Fc (see FIG. 5) will generate an Fc monomer which may be used in the complementary IgG-IgA pair below (SEQ ID NOs: 206 and 207).

| (SEQ ID NO: 206) | |
|---|---|
| 1 | THTCPPCPAP ELLGGPSVFL FPPKPKDTLM ISRTPEVTCV VVDVSHEDPE |
| 51 | VKFNWYVDGV EVHNAKTKPR EEQYNSTYRV VSVLTVLHQD WLNGKEYKCK |
| 101 | VSNKALPAPI EKTISKAKGQ PFRPEVHLLP PSREEMTKNQ VSLTCLARGF |
| 151 | YPKDIAVEWE SNGQPENNYK TTPSRQEPSO GTTTFAVTSK LTVDKSRWQQ |
| 201 | GNVFSCSVMH EALHNHYTQK TISLSPGK |
| (SEQ ID NO: 207) | |
| 1 | THTCPPCPAP ELLGGPSVFL FPPKPKDTLM ISRTPEVTCV VVDVSHEDPE |
| 51 | VKFNWYVDGV EVHNAKTKPR EEQYNSTYRV VSVLTVLHQD WLNGKEYKCK |
| 101 | VSNKALPAPI EKTISKAKGQ PREPQVYTLP PPSEELALNE LVTLTCLVKG |
| 151 | FYPSDIAVEW ESNGQELPRE KYLTWAPVLD SDGSFFLYSI LRVAAEDWKK |
| 201 | GDTFSCSVMH EALHNHYTQK SLDRSPGK |

In part, the disclosure provides desired pairing of asymmetric Fc-containing polypeptide chains with a cleavable leucine zipper domain attached at the C-terminus of the Fc C_{H}3 domains. Attachment of a leucine zipper is sufficient to cause preferential assembly of heterodimeric antibody heavy chains. See, e.g., Wranik et al (2012) J Biol Chem 287:43331-43339. As disclosed herein, one of a pair of Fc sequences attached to a leucine zipper-forming strand can be arbitrarily fused to the TGF-beta superfamily type I receptor polypeptide, type II receptor polypeptide, or co-receptor polypeptide of the construct, with or without an optional linker, to generate a TGF-beta superfamily fusion polypeptide. This single chain can be coexpressed in a cell of choice along with the Fc sequence attached to a complementary leucine zipper-forming strand to favor generation of the desired multichain construct. Proteolytic digestion of the construct with the bacterial endoproteinase Lys-C post purification can release the leucine zipper domain, resulting in an Fc construct whose structure is identical to that of native Fc. In this example based on leucine zipper pairing, SEQ ID NO: 213 [hG1Fc-Ap1 (acidic)] and SEQ ID NO: 214 [hG1Fc-Bp1 (basic)] are examples of complementary IgG Fc sequences in which the engineered complimentary leucine zipper sequences are underlined, and the TGF-beta superfamily type I/II polypeptide or co-receptor polypeptide of the construct can be fused to either SEQ ID NO: 213 or SEQ ID NO: 214, but not both. Given the high degree of amino acid sequence identity between native hG1Fc, native hG2Fc, native hG3Fc, and native hG4Fc, it can be appreciated that leucine zipper-forming sequences attached, with or without an optional linker, to hG1Fc, hG2Fc, hG3Fc, or hG4Fc (see FIG. 5) will generate an Fc monomer which may be used in the complementary leucine zipper-forming pair below (SEQ ID NOs: 213 and 214).

| (SEQ ID NO: 213) | |
|---|---|
| 1 | THTCPPCPAP ELLGGPSVFL FPPKPKDTLM ISRTPEVTCV VVDVSHEDPE |
| 51 | VKFNWYVDGV EVHNAKTKPR EEQYNSTYRV VSVLTVLHQD WLNGKEYKCK |
| 101 | VSNKALPAPI EKTISKAKGQ PREPQVYTLP PSREEMTKNQ VSLTCLVKGF |
| 151 | YPSDIAVEWE SNGQPENNYK TTPPVLDSDG SFFLYSKLTV DKSRWQQGNV |
| 201 | FSCSVMHEAL HNHYTQKSLS LSPGKGGSAQ LEKELQALEK ENAQLEWELQ |
| 251 | ALEKELAQGA T |
| (SEQ ID NO: 214) | |
| 1 | THTCPPCPAP ELLGGPSVFL FPPKPKDTLM ISRTPEVTCV VVDVSHEDPE |
| 51 | VKFNWYVDGV EVHNAKTKPR EEQYNSTYRV VSVLTVLHQD WLNGKEYKCK |
| 101 | VSNKALPAPI EKTISKAKGQ PREPQVYTLP PSREEMTKNQ VSLTCLVKGF |
| 151 | YPSDIAVEWE SNGQPENNYK TTPPVLDSDG SFFLYSKLTV DKSRWQQGNV |
| 201 | FSCSVMHEAL HNHYTQKSLS LSPGKGGSAQ LKKKLQALKK KNAQLKWKLQ |
| 251 | ALKKKLAQGA T |

In part, the disclosure provides desired pairing of asymmetric Fc-containing polypeptide chains by methods described above in combination with additional mutations in the Fc domain which facilitate purification of the desired heteromeric species. An example is complementarity of Fc domains based on knobs-into-holes pairing combined with an engineered disulfide bond, as disclosed in SEQ ID NOs: 204-205, plus additional substitution of two negatively charged amino acids (aspartic acid or glutamic acid) in one Fc-containing polypeptide chain and two positively charged amino acids (e.g., arginine) in the complementary Fc-containing polypeptide chain (SEQ ID NOs: 215-216). These four amino acid substitutions facilitate selective purification of the desired heteromeric fusion protein from a heterogeneous polypeptide mixture based on differences in isoelectric point or net molecular charge. The engineered amino acid substitutions in these sequences are double underlined below, and the TGFβ superfamily type I receptor polypeptide, type II receptor polypeptide, or co-receptor polypeptide of the construct can be fused to either SEQ ID NO: 215 or SEQ ID NO: 216, but not both. Given the high degree of amino acid sequence identity between native hG1Fc, native hG2Fc, native hG3Fc, and native hG4Fc, it can be appreciated that amino acid substitutions at corresponding positions in hG2Fc, hG3Fc, or hG4Fc (see FIG. 5) will generate complementary Fc pairs which may be used instead of the complementary hG1Fc pair below (SEQ ID NOs: 215-216).

| (SEQ ID NO: 215) | |
|---|---|
| 1 | THTCPPCPAP ELLGGPSVFL FPPKPKDTLM ISRTPEVTCV VVDVSHEDPE |
| 51 | VKFNWYVDGV EVHNAKTKPR EEQYNSTYRV VSVLTVLHQD WLNGKEYKCK |
| 101 | VSNKALPAPI EKTISKAKGQ PREPQVYTLP PCREEMTENQ VSLWCLVKGF |
| 151 | YPSDIAVEWE SNGQPENNYK TTPPVLDSDG SFFLYSKLTV DKSRWQQGNV |
| 201 | FSCSVMHEAL HNHYTQDSLS LSPGK |
| (SEQ ID NO: 216) | |
| 1 | THTCPPCPAP ELLGGPSVFL FPPKPKDTLM ISRTPEVTCV VVDVSHEDPE |
| 51 | VKFNWYVDGV EVHNAKTKPR EEQYNSTYRV VSVLTVLHQD WLNGKEYKCK |
| 101 | VSNKALPAPI EKTISKAKGQ PREPQVCTLP PSREEMTKNQ VSLSCAVKGF |
| 151 | YPSDIAVEWE SRGQPENNYK TTPPVLDSRG SFFLVSKLTV DKSRWQQGNV |
| 201 | FSCSVMHEAL HNHYTQKSLS LSPGK |

Another example involves complementarity of Fc domains based on knobs-into-holes pairing combined with an engineered disulfide bond, as disclosed in SEQ ID NOs: 204-205, plus a histidine-to-arginine substitution at position 213 in one Fc-containing polypeptide chain (SEQ ID NO: 217). This substitution (denoted H435R in the numbering system of Kabat et al.) facilitates separation of desired heteromer from undesirable homodimer based on differences in affinity for protein A. The engineered amino acid substitution is indicated by double underline, and the TGFβ superfamily type I receptor polypeptide, type II receptor polypeptide, or co-receptor polypeptide of the construct can be fused to either SEQ ID NO: 217 or SEQ ID NO: 205, but not both. Given the high degree of amino acid sequence identity between native hG1Fc, native hG2Fc, native hG3Fc, and native hG4Fc, it can be appreciated that amino acid substitutions at corresponding positions in hG2Fc, hG3Fc, or hG4Fc (see FIG. 5) will generate complementary Fc pairs which may be used instead of the complementary hG1Fc pair of SEQ ID NO: 217 (below) and SEQ ID NO: 205.

| (SEQ ID NO: 217) | |
|---|---|
| 1 | THTCPPCPAP ELLGGPSVFL FPPKPKDTLM ISRTPEVTCV VVDVSHEDPE |
| 51 | VKFNWYVDGV EVHNAKTKPR EEQYNSTYRV VSVLTVLHQD WLNGKEYKCK |
| 101 | VSNKALPAPI EKTISKAKGQ PREPQVYTLP PCREEMTKNQ VSLWCLVKGF |
| 151 | YPSDIAVEWE SNGQPENNYK TTPPVLDSDG SFFLYSKLTV DKSRWQQGNV |
| 201 | FSCSVMHEAL HNRYTQKSLS LSPGK |

A variety of engineered mutations in the Fc domain are presented above with respect to the G1Fc sequence (SEQ ID NO: 208). Analogous mutations in G2Fc, G3Fc, and G4Fc can be derived from their alignment with G1Fc in FIG. 5. Due to unequal hinge lengths, analogous Fc positions based on isotype alignment (FIG. 5) possess different amino acid numbers in SEQ ID NOs: 208, 209, 210, and 212 as summarized in the following table.

Correspondence between
C_{H}3 Positions for Human Fc Isotypes*

| IgG1 | IgG4 | IgG2 | IgG3 |
|---|---|---|---|
| SEQ ID | SEQ ID | SEQ ID | SEQ ID |
| NO: 208 | NO: 212 | NO: 209 | NO: 210 |
| Numbering | Numbering | Numbering | Numbering |
| begins | begins | begins | begins |
| at THT . . . | at ESK . . . | at VEC . . . | at EPK . . . |
| Y127 | Y131 | Y125 | Y134 |
| S132 | S136 | S130 | S139 |
| E134 | E138 | E132 | E141 |
| K138 | K142 | K136 | K145 |
| T144 | T148 | T142 | T151 |
| L146 | L150 | L144 | L153 |
| N162 | N166 | N160 | S169 |
| K170 | K174 | K168 | N177 |
| D177 | D181 | D175 | D184 |
| D179 | D183 | D177 | D186 |
| Y185 | Y189 | Y183 | Y192 |
| K187 | R191 | K185 | K194 |
| H213 | H217 | H211 | R220 |
| K217 | K221 | K215 | K224 |

*Numbering based on multiple sequence alignment shown in FIG. 5

As described above, various methods are known in the art that increase desired pairing of Fc-containing fusion polypeptide chains in a single cell line to produce a preferred asymmetric fusion protein at acceptable yields [Klein et al (2012) mAbs 4:653-663; and Spiess et al (2015) Molecular Immunology 67(2A): 95-106]. In addition, heteromultimers as described herein may be generated using a combination of heavy and light chain fusion proteins comprising either an TGF-beta superfamily type I receptor polypeptide, type II receptor polypeptide, or co-receptor polypeptide. For example, in some embodiments, a TGF-beta superfamily type I or type II receptor polypeptide may be fused, with or without a linker domain, to an immunoglobulin heavy chain (IgG1, IgG2, IgG3, IgG4, IgM, IgA1, or IgA2) that comprises at least a portion of the C_{H}1 domain. Similarly, a TGF-beta superfamily co-receptor polypeptide may be fused, with or without a linker domain, to an immunoglobulin light chain (kappa or lambda) that comprises at least a portion of the light chain constant domain (C_{L}). In alternative embodiments, a TGF-beta superfamily co-receptor polypeptide may be fused, with or without a linker domain, to an immunoglobulin heavy chain (IgG1, IgG2, IgG3, IgG4, IgM, IgA1, or IgA2) that comprises at least a portion of the C_{H}1 domain, and a TGF-beta superfamily type I receptor or type II receptor polypeptide may be fused, with or without a linker domain, to an immunoglobulin light chain (kappa or lambda) that comprises at least a portion of the light chain constant domain (C_{L}). This design takes advantage of the natural ability of the heavy chains to heterodimerize with light chains. In particular, heterodimerization of a heavy and light chain occurs between the C_{H}1 with the C_{L}, which is generally stabilized by covalent linking of the two domains via a disulfide bridge. Constructs employing the full-length heavy chain, or at least a portion of the heavy chain comprising the hinge region, could give rise to antibody-like molecules comprising two “light chains” and two “heavy chains”. See FIG. 7. A potential advantage of this design is that it may more closely mimic the naturally occurring TGF-beta superfamily type I/II receptor polypeptide-ligand-TGF-beta superfamily co-receptor polypeptide complex and may display higher affinity for the ligand than comparable single homodimers. In some embodiments, this design may be modified by incorporating various heavy chain truncations including, for example, truncations that comprise the C_{H}1 domain and some or all of the hinge domain (giving rise to F(ab′)₂-like molecules) as well as truncations that only comprise the C_{H}1 domain or a fragment thereof (giving rise to Fab-like molecules). See FIG. 7G. Various methods for designing such heteromultimer constructs are described in US 2009/0010879, Klein et al [(2012) mAbs 4:653-663], and Spiess et al [(2015) Molecular Immunology 67(2A): 95-106] the contents of which are incorporated in their entirety herein.

In some embodiments, it is desirable to generate antibody-like heterodimers comprising at least one branch of the complex comprising an TGF-beta superfamily type I or type II receptor polypeptide-C_{L}:TGF-beta superfamily co-receptor polypeptide-C_{H}1 heterodimer pair and at least a second branch comprising an TGF-beta superfamily co-receptor polypeptide-C_{L}:TGF-beta superfamily type I or type II receptor polypeptide r-C_{H}1 heterodimer pair. See, e.g., FIG. 7B. Such heterodimer complexes can be generated, for example, using combinations of heavy chain and light chain asymmetrical pairing technologies [Spiess et al (2015) Molecular Immunology 67(2A): 95-106]. For example, in CrossMab technology, [Schaefer et al (2011). Proc. Natl. Acad. Sci. U.S.A. 108: 11187-11192] light chain mispairing is overcome using domain crossovers and heavy chains heterodimerized using knobs-into-holes [Merchant et al (1998) Nat. Biotechnol. 16: 677-681]. For the domain crossovers either the variable domains or the constant domains are swapped between light and heavy chains to create two asymmetric Fab arms that drive cognate light chain pairing while preserving the structural and functional integrity of the variable domain [Fenn et al (2013) PLoS ONE 8: e61953]. An alternative approach for overcoming light chain mispairing is designing heavy and light chains with orthogonal Fab inter-faces [Lewis (2014) Nat. Biotechnol. 32: 191-198]. This has been accomplished by computational modeling [Das et al (2008) Annu. Rev. Biochem. 77: 363-382] in combination with X-ray crystallography to identify mutations at the V_{H}/V_{L} and C_{H}1/C_{L} interfaces. For the heterodimers generated using this methodology, it may be necessary to engineer mutations into both V_{H}/V_{L} and C_{H}1/C_{L} interfaces to minimize heavy/light chain mispairing. The designed orthogonal Fab interface may be used in conjunction with a heavy chain heterodimerization strategy to facilitate efficient IgG production in a single host cell. Electrostatic steering may also be used to generate orthogonal Fab interfaces to facilitate the construction of such heterodimers. Peptide linkers may be used to ensure cognate pairing of light and heavy chains in a format known as “LUZ-Y” [Wranik et al (2012) J. Biol. Chem. 287: 43331-43339], wherein heavy chain heterodimerization is accomplished using leucine zippers which may be subsequently removed by proteolysis in vitro.

Alternatively, heteromultimers may comprise one or more single-chain ligand traps as described herein, optionally which may be covalently or non-covalently associated with one or more TGF-beta superfamily type I receptor polypeptides, type II receptor polypeptides, or co-receptor polypeptides as well as additional TGF-beta superfamily type I/II receptor polypeptide:TGF-beta superfamily co-receptor polypeptide single chain ligand traps [US 2011/0236309 and US2009/0010879]. See FIGS. 9 and 10. As described herein, single-chain ligand traps do not require fusion to any multimerization domain such as coiled-coil Fc domains to be multivalent. In general, single-chain ligand traps of the present disclosure comprise at least one TGF-beta superfamily type I receptor polypeptide or type II receptor polypeptide domain and one TGF-beta superfamily co-receptor polypeptide domain. The TGF-beta superfamily type I or type II receptor polypeptide and TGF-beta superfamily co-receptor polypeptide domains, generally referred to herein as binding domains (BD), optionally may be joined by a linker region.

For example, in one aspect, the present disclosure provides heteromultimers comprising a polypeptide having the following structure:

(<BD1>-linker1)ₖ-[<BD2>-linker2-{<BD3>-linker3}_{f}]ₙ-(<BD4>)ₘ-(linker4-BD5>_{d})ₕ

where: n and h are independently greater than or equal to one; d, f, m, and k are independently equal to or greater than zero; BD1, BD2, BD3, BD4, and BD5 are independently TGF-beta superfamily type I/II receptor polypeptide or TGF-beta superfamily co-receptor polypeptide domains, wherein at least one of BD1, BD2, BD3, and BD4 is an TGF-beta superfamily type I/II receptor polypeptide domain, and wherein at least one of BD1, BD2, BD3, and BD4 is an TGF-beta superfamily co-receptor polypeptide domain, and linker1, linker2, linker3, and linker 4 are independently greater than or equal to zero. In some embodiment, TGF-beta superfamily type I/II receptor polypeptide:TGF-beta superfamily co-receptor polypeptide single-chain traps comprise at least two different TGF-beta superfamily type I or type II receptor polypeptide. In some embodiments, TGF-beta superfamily type I/II receptor polypeptide:TGF-beta superfamily co-receptor polypeptide single-chain traps comprise at least two different TGF-beta superfamily co-receptor polypeptide polypeptides. In some embodiment, TGF-beta superfamily type I/II receptor polypeptide:TGF-beta superfamily co-receptor polypeptide single-chain traps comprise at least two different linkers. Depending on the values of selected for d, f, h, k, m, and n, the heteromultimer structure may comprise a large number of repeating units in various combinations or may be a relatively simple structure.

In another aspect, the present disclosure provides heteromultimers comprising a polypeptide having the following structure:

<BD1>-linker1-<BD2>

In yet another aspect, the present disclosure provides heteromultimers comprising a polypeptide having the following structure:

<BD1>-(linker2-<BD2>)ₙ

where n is greater than or equal one.

Another aspect of the invention provides heteromultimers comprising a polypeptide having the following structure:

(<BD1>-linker1-<BD1>)_{f}-linker2-(<BD2>-linker3-<BD3>)_{g}

wherein f and g are greater than or equal to one.

In an embodiment where BD2 and BD3 are the same, and f and g are the same number, this can result in a substantially mirror symmetric structure around linker 2, subject to differences in the linkers. In instances where BD2 is different from BD3 and/or where f and g are different numbers, different structures will be produced. It is within the capacity of one of ordinary skill in the art to select suitable binding domains, linkers, and repeat frequencies in light of the disclosure herein and knowledge in the art. Specific, non-limiting examples of such single-chain ligand traps in accordance with the present disclosure are represented schematically in FIG. 9.

The linkers (1, 2, 3, and 4) may be the same or different. The linker region provides a segment that is distinct from the structured ligand-binding domains of TGF-beta superfamily type I/II receptor polypeptide and TGF-beta superfamily co-receptor polypeptide and thus can be used for conjugation to accessory molecules (e.g., molecules useful in increasing stability such as PEGylation moieties) without having to chemically modify the binding domains. The linker may include an unstructured amino acid sequence that may be either the same as or derived from conservative modifications to the sequence of a natural unstructured region in the extracellular portion of the receptor for the ligand of interest or another receptor in the TGF-β superfamily. In other instances, such linkers may be entirely artificial in composition and origin but will contain amino acids selected to provide an unstructured flexible linker with a low likelihood of encountering electrostatic or steric hindrance complications when brought into close proximity to the ligand of interest. Linker length will be considered acceptable when it permits binding domains located on each of the N- and C-termini of the linker to bind their natural binding sites on their natural ligand such that, with both binding domains so bound, the ligand is bound with a higher affinity than it would be bound by binding of only one of the binding domains. In some instances, the number of amino acid residues in the linker of either natural or artificial origin is selected to be equal to or greater than the minimum required distance for simultaneous (bridged) binding to two binding sites on the TGF-beta superfamily type I/II receptor polypeptide and/or TGF-beta superfamily co-receptor polypeptide ligand. For example, and without wishing to be limiting in any manner, the linker length may be between about 1-10 amino acids, 10-20 amino acids, 18-80 amino acids, 25-60 amino acids, 35-45 amino acids, or any other suitable length.

Linkers may be designed to facilitate purification of the polypeptide. The exact purification scheme chosen will determine what modifications are needed, for example and without wishing to be limiting, additions of purification “tags” such as His tags is contemplated; in other examples, the linker may include regions to facilitate the addition of cargo or accessory molecules. When such additions affect the unstructured nature of the linker or introduce potential electrostatic or steric concerns, appropriate increases to the linker length will be made to ensure that the two binding domains are able to bind their respective sites on the ligand. In light of the methods and teachings herein, such determinations could be made routinely by one skilled in the art.

In addition, the present design permits linkage of other cargo molecules (for example imaging agents like fluorescent molecules), toxins, etc. For example, and without wishing to be limiting in any manner, single-chain polypeptides can be modified to add one or more cargo and/or accessory molecules (referred to collectively herein by R1, R2, R3, R4, etc.):

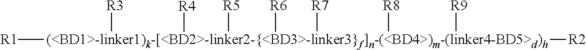

Without limiting the generality of R substituents available, R1, R2, R3, R4, R5, R6, R7, R8, R9, may or may not be present; when present, they may be the same or different, and may independently be one or more of: a fusion protein for targeting, for example, but not limited to such as an antibody fragment (e.g. single chain Fv) and/or a single domain antibody (sdAb); a radiotherapy and/or imaging agent, for example, but not limited to a radionucleotide (e.g. ¹²³I, ¹¹¹In, ¹⁸F, ⁶⁴C, ⁶⁸Y, ¹²⁴I, ¹³¹I, ⁹⁰Y, ¹⁷⁷Lu, ⁵⁷Cu, ²¹³Bi, ²¹¹At), a fluorescent dye (e.g. Alexa Fluor, Cy dye) and/or a fluorescent protein tag (e.g. GFP, DsRed); a cytotoxic agent for chemotherapy, for example, but not limited to doxorubicin, calicheamicin, a maytansinoid derivatives (e.g. DM1, DM4), a toxin (eg. truncated Pseudomonas endotoxin A, diphtheria toxin); a nanoparticle-based carrier, for example, but not limited to polyethylene glycol (PEG), a polymer-conjugated to drug, nanocarrier or imaging agent (e.g. of a polymer N-(2-hydroxylpropyl) methacrylamide (HPMA), glutamic acid, PEG, dextran); a drug (for example, but not limited to doxorubicin, camptothecin, paclitaxel, palatinate); a nanocarrier, for example, but not limited to a nanoshell or liposome; an imaging agent, for example, but not limited to Supermagnetic Iron Oxide (SPIO); a dendrimer; and/or a solid support for use in ligand purification, concentration or sequestration (e.g. nanoparticles, inert resins, suitable silica supports).

In general, it will not be preferable to have cargo or accessory molecules in all possible positions, as this may cause steric or electrostatic complications. However, the effects of adding a cargo or accessory molecule to any given position or positions on the structure can be determined routinely in light of the disclosure herein by modeling the linker between the binding domains and carrying out molecular dynamics simulations to substantially minimize molecular mechanics energy and reduce steric and electrostatic incompatibility between the linker and the TGF-beta superfamily type I/II receptor polypeptide and TGF-beta superfamily co-receptor polypeptide as taught herein.

It may be preferable to add the cargo or accessory molecule to the linker portion of the agent, rather to the binding domain, to reduce the likelihood of interference in binding function. However, addition to the binding domain is possible and could be desirable in some instances and the effect of such an addition can be determined routinely in advance by modeling the binding agent and the linker with the proposed addition as described herein.

Conjugation methodologies may be performed using commercial kits that enable conjugation via common reactive groups such as primary amines, succinimidyl (NHS) esters and sulfhydral-reactive groups. Some non-limiting examples are: Alexa Fluor 488 protein labeling kit (Molecular Probes, Invitrogen detection technologies) and PEGylation kits (Pierce Biotechnology Inc.).

In certain aspects, TGF-beta superfamily type I or type II receptor polypeptide:TGF-beta superfamily co-receptor polypeptide single-chain traps may be covalently or non-covalently associated with one or more TGF-beta superfamily type I/II receptor polypeptides or TGF-beta superfamily co-receptor polypeptide as well as additional TGF-beta superfamily type I/II receptor polypeptide:TGF-beta superfamily co-receptor polypeptide single chain ligand traps to form higher order heteromultimers, which may be used in accordance with the methods described herein. See, e.g., FIG. 10. For example, an TGF-beta superfamily type I or type II receptor polypeptide:TGF-beta superfamily co-receptor polypeptide single chain ligand trap may further comprise a multimerization domain as described herein. In some embodiments, TGF-beta superfamily type I or type II receptor polypeptide:TGF-beta superfamily co-receptor polypeptide single chain ligand traps comprise a constant domain of an Ig immunoglobulin. Such immunoglobulins constant domains may be selected to promote symmetrical or asymmetrical complexes comprising at least one single-chain TGF-beta superfamily type I or type II receptor polypeptide:TGF-beta superfamily co-receptor polypeptide trap.

In certain aspects, a TGF-beta superfamily type I or type II receptor polypeptide:TGF-beta superfamily co-receptor polypeptide single-chain trap, or combinations of such traps, may be used as TGF-beta superfamily antagonists to treat or prevent an TGF-beta superfamily disorder or disease as described herein (e.g., a bone-related disorder and anemia).

It is understood that different elements of the fusion proteins (e.g., immunoglobulin Fc fusion proteins) may be arranged in any manner that is consistent with desired functionality. For example, a TGF-beta superfamily type I receptor polypeptide, type II receptor polypeptide, or co-receptor polypeptide domain may be placed C-terminal to a heterologous domain, or alternatively, a heterologous domain may be placed C-terminal to a TGF-beta superfamily type I receptor polypeptide, type II receptor polypeptide, and/or co-receptor polypeptide domain. The TGF-beta superfamily type I receptor polypeptide, type II receptor polypeptide, or co-receptor domain and the heterologous domain need not be adjacent in a fusion protein, and additional domains or amino acid sequences may be included C- or N-terminal to either domain or between the domains.

For example, a TGF-beta superfamily type I receptor, type II receptor, or co-receptor fusion protein may comprise an amino acid sequence as set forth in the formula A-B-C. The B portion corresponds to a TGF-beta superfamily type I receptor polypeptide, type II receptor polypeptide, or co-receptor polypeptide domain. The A and C portions may be independently zero, one, or more than one amino acid, and both the A and C portions when present are heterologous to B. The A and/or C portions may be attached to the B portion via a linker sequence. A linker may be rich in glycine (e.g., 2-10, 2-5, 2-4, 2-3 glycine residues) or glycine and proline residues and may, for example, contain a single sequence of threonine/serine and glycines or repeating sequences of threonine/serine and/or glycines, e.g., GGG (SEQ ID NO: 58), GGGG (SEQ ID NO: 59), TGGGG (SEQ ID NO: 60), SGGGG (SEQ ID NO: 61), TGGG (SEQ ID NO: 62), or SGGG (SEQ ID NO: 63) singlets, or repeats. In certain embodiments, a TGF-beta superfamily type I receptor, type II receptor, or co-receptor fusion protein comprises an amino acid sequence as set forth in the formula A-B-C, wherein A is a leader (signal) sequence, B consists of a TGF-beta superfamily type I receptor polypeptide, type II receptor polypeptide, or co-receptor polypeptide domain, and C is a polypeptide portion that enhances one or more of in vivo stability, in vivo half-life, uptake/administration, tissue localization or distribution, formation of protein complexes, and/or purification. In certain embodiments, a TGF-beta superfamily type I receptor, type II receptor, or co-receptor fusion protein comprises an amino acid sequence as set forth in the formula A-B-C, wherein A is a TPA leader sequence, B consists of a TGF-beta superfamily type I receptor polypeptide, type II receptor polypeptide, or co-receptor polypeptide domain, and C is an immunoglobulin Fc domain. Preferred fusion proteins comprise the amino acid sequence set forth in any one of SEQ ID NOs: 101, 103, 104, 106, 107, 109, 601, 602, 603, 604, 605, 606, 801, 802, 803, 804, 805, 806, 901, 902, 903, 904, 905, and 906.

In some embodiments, heteromultimers of the present disclosure further comprise one or more heterologous portions (domains) so as to confer a desired property. For example, some fusion domains are particularly useful for isolation of the fusion proteins by affinity chromatography. Well-known examples of such fusion domains include, but are not limited to, polyhistidine, Glu-Glu, glutathione S-transferase (GST), thioredoxin, protein A, protein G, an immunoglobulin heavy-chain constant region (Fc), maltose binding protein (MBP), or human serum albumin. For the purpose of affinity purification, relevant matrices for affinity chromatography, such as glutathione-, amylase-, and nickel- or cobalt-conjugated resins are used. Many of such matrices are available in “kit” form, such as the Pharmacia GST purification system and the QIAexpress™ system (Qiagen) useful with (HIS₆) fusion partners. As another example, a fusion domain may be selected so as to facilitate detection of the ligand trap polypeptides. Examples of such detection domains include the various fluorescent proteins (e.g., GFP) as well as “epitope tags,” which are usually short peptide sequences for which a specific antibody is available. Well-known epitope tags for which specific monoclonal antibodies are readily available include FLAG, influenza virus hemagglutinin (HA), and c-myc tags. In some cases, the fusion domains have a protease cleavage site, such as for factor Xa or thrombin, which allows the relevant protease to partially digest the fusion proteins and thereby liberate the recombinant proteins therefrom. The liberated proteins can then be isolated from the fusion domain by subsequent chromatographic separation.

In certain embodiments, TGF-beta superfamily type I receptor polypeptides, type II receptor polypeptides, and/or co-receptor polypeptides of the present disclosure contain one or more modifications that are capable of stabilizing the polypeptides. For example, such modifications enhance the in vitro half-life of the polypeptides, enhance circulatory half-life of the polypeptides, and/or reduce proteolytic degradation of the polypeptides. Such stabilizing modifications include, but are not limited to, fusion proteins (including, for example, fusion proteins comprising a type I receptor polypeptide, type II receptor polypeptide, or co-receptor polypeptide domain and a stabilizer domain), modifications of a glycosylation site (including, for example, addition of a glycosylation site to a polypeptide of the disclosure), and modifications of carbohydrate moiety (including, for example, removal of carbohydrate moieties from a polypeptide of the disclosure). As used herein, the term “stabilizer domain” not only refers to a fusion domain (e.g., an immunoglobulin Fc domain) as in the case of fusion proteins, but also includes nonproteinaceous modifications such as a carbohydrate moiety, or nonproteinaceous moiety, such as polyethylene glycol.

In preferred embodiments, heteromultimers to be used in accordance with the methods described herein are isolated polypeptide complexes. As used herein, an isolated protein (or protein complex) or polypeptide (or polypeptide complex) is one which has been separated from a component of its natural environment. In some embodiments, a heteromultimer complex of the disclosure is purified to greater than 95%, 96%, 97%, 98%, or 99% purity as determined by, for example, electrophoretic (e.g., SDS-PAGE, isoelectric focusing (IEF), capillary electrophoresis) or chromatographic (e.g., ion exchange or reverse phase HPLC). Methods for assessment of antibody purity are well known in the art [See, e.g., Flatman et al., (2007) J. Chromatogr. B 848:79-87]. In some embodiments, heteromultimer preparations of the disclosure are substantially free of TGF-beta superfamily type I receptor polypeptide homomultimers, TGF-beta superfamily type II receptor polypeptide homomultimers, and/or TGF-beta superfamily co-receptor polypeptide homomultimers. For example, in some embodiments, heteromultimer preparations comprise less than about 10%, 9%, 8%, 7%, 5%, 4%, 3%, 2%, or less than 1% of TGF-beta superfamily type I receptor polypeptide homomultimers. In some embodiments, heteromultimer preparations comprise less than about 10%, 9%, 8%, 7%, 5%, 4%, 3%, 2%, or less than 1% of TGF-beta superfamily type II receptor polypeptide homomultimers. In some embodiments, heteromultimer preparations comprise less than about 10%, 9%, 8%, 7%, 5%, 4%, 3%, 2%, or less than 1% of TGF-beta superfamily co-receptor polypeptide homomultimers. In some embodiments, heteromultimer preparations comprise less than about 10%, 9%, 8%, 7%, 5%, 4%, 3%, 2%, or less than 1% of TGF-beta superfamily type I receptor polypeptide homomultimers and less than about 10%, 9%, 8%, 7%, 5%, 4%, 3%, 2%, or less than 1% of TGF-beta superfamily co-receptor polypeptide homomultimers. In some embodiments, heteromultimer preparations comprise less than about 10%, 9%, 8%, 7%, 5%, 4%, 3%, 2%, or less than 1% of TGF-beta superfamily type II receptor polypeptide homomultimers and less than about 10%, 9%, 8%, 7%, 5%, 4%, 3%, 2%, or less than 1% of TGF-beta superfamily co-receptor polypeptide homomultimers.

In certain embodiments, TGFβ superfamily type I receptor polypeptides, type II receptor polypeptides, and co-receptor polypeptides as well as heteromultimer complexes thereof, of the disclosure can be produced by a variety of art-known techniques. For example, polypeptides of the disclosure can be synthesized using standard protein chemistry techniques such as those described in Bodansky, M. Principles of Peptide Synthesis, Springer Verlag, Berlin (1993) and Grant G. A. (ed.), Synthetic Peptides: A User's Guide, W. H. Freeman and Company, New York (1992). In addition, automated peptide synthesizers are commercially available (see, e.g., Advanced ChemTech Model 396; Milligen/Biosearch 9600). Alternatively, the polypeptides and complexes of the disclosure, including fragments or variants thereof, may be recombinantly produced using various expression systems [e.g., E. coli, Chinese Hamster Ovary (CHO) cells, COS cells, baculovirus] as is well known in the art. In a further embodiment, the modified or unmodified polypeptides of the disclosure may be produced by digestion of recombinantly produced full-length TGFβ superfamily type I receptor, type II receptor and/or co-receptor polypeptides by using, for example, a protease, e.g., trypsin, thermolysin, chymotrypsin, pepsin, or paired basic amino acid converting enzyme (PACE). Computer analysis (using a commercially available software, e.g., MacVector, Omega, PCGene, Molecular Simulation, Inc.) can be used to identify proteolytic cleavage sites.

### B. Nucleic Acids Encoding TGFβ Superfamily Type I Receptor Polypeptides, Type II Receptor Polypeptides, and Co-Receptor Polypeptides

In certain embodiments, the present disclosure provides isolated and/or recombinant nucleic acids encoding TGFβ superfamily type I receptors, type II receptors, and co-receptors (including fragments, functional variants, and fusion proteins thereof) disclosed herein. For example, SEQ ID NO: 12 encodes a naturally occurring human ActRIIA precursor polypeptide, while SEQ ID NO: 13 encodes a mature, extracellular domain of ActRIIA. The subject nucleic acids may be single-stranded or double stranded. Such nucleic acids may be DNA or RNA molecules. These nucleic acids may be used, for example, in methods for making TGF-beta superfamily heteromultimers of the present disclosure.

In certain embodiments, nucleic acids encoding TGFβ superfamily type I receptor polypeptides, type II receptor polypeptides, and/or co-receptor polypeptides of the present disclosure are understood to include nucleic acids of any one of SEQ ID NOs: 7, 8, 12, 13, 16, 17, 20, 21, 24, 25, 28, 29, 32, 33, 36, 37, 40, 41, 44, 45, 48, 49, 52, 53, 69, 70, 73, 74, 77, 78, 81, 82, 85, 86, 89, 90, 93, 94, 303, 304, 307, 308, 311, 312, 503, 504, 507, 508, 511, 512, 515, 516, 519, 520, 523, 524, 527, 528, 531, 532, 535, 536, 539, 540, 543, 544, 547, 548, 551, 552, 555, 556, 559, 560, 563, 564, 567, 568, 571, 572, 575, 576, 579, 580, 583, 584, 587, 588, 591, 592, 594, 597, 598, 601, 602, 605, 606, 102, 105, 108, 808, and 811 as well as variants thereof. Variant nucleotide sequences include sequences that differ by one or more nucleotide substitutions, additions, or deletions including allelic variants, and therefore, will include coding sequences that differ from the nucleotide sequence designated in any one of SEQ ID NOs: 7, 8, 12, 13, 16, 17, 20, 21, 24, 25, 28, 29, 32, 33, 36, 37, 40, 41, 44, 45, 48, 49, 52, 53, 69, 70, 73, 74, 77, 78, 81, 82, 85, 86, 89, 90, 93, 94, 303, 304, 307, 308, 311, 312, 503, 504, 507, 508, 511, 512, 515, 516, 519, 520, 523, 524, 527, 528, 531, 532, 535, 536, 539, 540, 543, 544, 547, 548, 551, 552, 555, 556, 559, 560, 563, 564, 567, 568, 571, 572, 575, 576, 579, 580, 583, 584, 587, 588, 591, 592, 594, 597, 598, 601, 602, 605, 606, 102, 105, 108, 808, and 811.

In certain embodiments, TGFβ superfamily type I receptor polypeptides, type II receptor polypeptides, and/or co-receptor polypeptides of the present disclosure are encoded by isolated or recombinant nucleic acid sequences that are at least 70%, 75%, 80%, 85%, 90%, 91%, 92%, 93%, 94%, 95%, 96%, 97%, 98%, 99%, or 100% identical to SEQ ID NOs: 7, 8, 12, 13, 16, 17, 20, 21, 24, 25, 28, 29, 32, 33, 36, 37, 40, 41, 44, 45, 48, 49, 52, 53, 69, 70, 73, 74, 77, 78, 81, 82, 85, 86, 89, 90, 93, 94, 303, 304, 307, 308, 311, 312, 503, 504, 507, 508, 511, 512, 515, 516, 519, 520, 523, 524, 527, 528, 531, 532, 535, 536, 539, 540, 543, 544, 547, 548, 551, 552, 555, 556, 559, 560, 563, 564, 567, 568, 571, 572, 575, 576, 579, 580, 583, 584, 587, 588, 591, 592, 594, 597, 598, 601, 602, 605, 606, 102, 105, 108, 808, and 811. One of ordinary skill in the art will appreciate that nucleic acid sequences that are at least 70%, 75%, 80%, 85%, 90%, 91%, 92%, 93%, 94%, 95%, 96%, 97%, 98%, 99%, or 100% identical to the sequences complementary to SEQ ID NOs: 7, 8, 12, 13, 16, 17, 20, 21, 24, 25, 28, 29, 32, 33, 36, 37, 40, 41, 44, 45, 48, 49, 52, 53, 69, 70, 73, 74, 77, 78, 81, 82, 85, 86, 89, 90, 93, 94, 303, 304, 307, 308, 311, 312, 503, 504, 507, 508, 511, 512, 515, 516, 519, 520, 523, 524, 527, 528, 531, 532, 535, 536, 539, 540, 543, 544, 547, 548, 551, 552, 555, 556, 559, 560, 563, 564, 567, 568, 571, 572, 575, 576, 579, 580, 583, 584, 587, 588, 591, 592, 594, 597, 598, 601, 602, 605, 606, 102, 105, 108, 808, and 811 are also within the scope of the present disclosure. In further embodiments, the nucleic acid sequences of the disclosure can be isolated, recombinant, and/or fused with a heterologous nucleotide sequence or in a DNA library.

In other embodiments, nucleic acids of the present disclosure also include nucleotide sequences that hybridize under highly stringent conditions to the nucleotide sequence designated in SEQ ID NOs: 7, 8, 12, 13, 16, 17, 20, 21, 24, 25, 28, 29, 32, 33, 36, 37, 40, 41, 44, 45, 48, 49, 52, 53, 69, 70, 73, 74, 77, 78, 81, 82, 85, 86, 89, 90, 93, 94, 303, 304, 307, 308, 311, 312, 503, 504, 507, 508, 511, 512, 515, 516, 519, 520, 523, 524, 527, 528, 531, 532, 535, 536, 539, 540, 543, 544, 547, 548, 551, 552, 555, 556, 559, 560, 563, 564, 567, 568, 571, 572, 575, 576, 579, 580, 583, 584, 587, 588, 591, 592, 594, 597, 598, 601, 602, 605, 606, 102, 105, 108, 808, and 811, the complement sequence of SEQ ID NOs: 7, 8, 12, 13, 16, 17, 20, 21, 24, 25, 28, 29, 32, 33, 36, 37, 40, 41, 44, 45, 48, 49, 52, 53, 69, 70, 73, 74, 77, 78, 81, 82, 85, 86, 89, 90, 93, 94, 303, 304, 307, 308, 311, 312, 503, 504, 507, 508, 511, 512, 515, 516, 519, 520, 523, 524, 527, 528, 531, 532, 535, 536, 539, 540, 543, 544, 547, 548, 551, 552, 555, 556, 559, 560, 563, 564, 567, 568, 571, 572, 575, 576, 579, 580, 583, 584, 587, 588, 591, 592, 594, 597, 598, 601, 602, 605, 606, 102, 105, 108, 808, and 811, or fragments thereof. One of ordinary skill in the art will understand readily that appropriate stringency conditions which promote DNA hybridization can be varied. For example, one could perform the hybridization at 6.0× sodium chloride/sodium citrate (SSC) at about 45° C., followed by a wash of 2.0×SSC at 50° C. For example, the salt concentration in the wash step can be selected from a low stringency of about 2.0×SSC at 50° C. to a high stringency of about 0.2×SSC at 50° C. In addition, the temperature in the wash step can be increased from low stringency conditions at room temperature, about 22° C., to high stringency conditions at about 65° C. Both temperature and salt may be varied, or temperature or salt concentration may be held constant while the other variable is changed. In one embodiment, the disclosure provides nucleic acids which hybridize under low stringency conditions of 6×SSC at room temperature followed by a wash at 2×SSC at room temperature.

Isolated nucleic acids which differ from the nucleic acids as set forth in SEQ ID NOs: 7, 8, 12, 13, 16, 17, 20, 21, 24, 25, 28, 29, 32, 33, 36, 37, 40, 41, 44, 45, 48, 49, 52, 53, 69, 70, 73, 74, 77, 78, 81, 82, 85, 86, 89, 90, 93, 94, 303, 304, 307, 308, 311, 312, 503, 504, 507, 508, 511, 512, 515, 516, 519, 520, 523, 524, 527, 528, 531, 532, 535, 536, 539, 540, 543, 544, 547, 548, 551, 552, 555, 556, 559, 560, 563, 564, 567, 568, 571, 572, 575, 576, 579, 580, 583, 584, 587, 588, 591, 592, 594, 597, 598, 601, 602, 605, 606, 102, 105, 108, 808, and 811 due to degeneracy in the genetic code are also within the scope of the disclosure. For example, a number of amino acids are designated by more than one triplet. Codons that specify the same amino acid, or synonyms (for example, CAU and CAC are synonyms for histidine) may result in “silent” mutations which do not affect the amino acid sequence of the protein. However, it is expected that DNA sequence polymorphisms that do lead to changes in the amino acid sequences of the subject proteins will exist among mammalian cells. One skilled in the art will appreciate that these variations in one or more nucleotides (up to about 3-5% of the nucleotides) of the nucleic acids encoding a particular protein may exist among individuals of a given species due to natural allelic variation. Any and all such nucleotide variations and resulting amino acid polymorphisms are within the scope of this disclosure.

In certain embodiments, the recombinant nucleic acids of the present disclosure may be operably linked to one or more regulatory nucleotide sequences in an expression construct. Regulatory nucleotide sequences will generally be appropriate to the host cell used for expression. Numerous types of appropriate expression vectors and suitable regulatory sequences are known in the art for a variety of host cells. Typically, said one or more regulatory nucleotide sequences may include, but are not limited to, promoter sequences, leader or signal sequences, ribosomal binding sites, transcriptional start and termination sequences, translational start and termination sequences, and enhancer or activator sequences. Constitutive or inducible promoters as known in the art are contemplated by the disclosure. The promoters may be either naturally occurring promoters, or hybrid promoters that combine elements of more than one promoter. An expression construct may be present in a cell on an episome, such as a plasmid, or the expression construct may be inserted in a chromosome. In some embodiments, the expression vector contains a selectable marker gene to allow the selection of transformed host cells. Selectable marker genes are well known in the art and will vary with the host cell used.

In certain aspects of the present disclosure, the subject nucleic acid is provided in an expression vector comprising a nucleotide sequence encoding a TGFβ superfamily type I receptor polypeptide, type II receptor polypeptide, and/or co-receptor polypeptide and operably linked to at least one regulatory sequence. Regulatory sequences are art-recognized and are selected to direct expression of the TGFβ superfamily type I receptor polypeptide, type II receptor polypeptide, and/or co-receptor polypeptide. Accordingly, the term regulatory sequence includes promoters, enhancers, and other expression control elements. Exemplary regulatory sequences are described in Goeddel; Gene Expression Technology: Methods in Enzymology, Academic Press, San Diego, Calif. (1990). For instance, any of a wide variety of expression control sequences that control the expression of a DNA sequence when operatively linked to it may be used in these vectors to express DNA sequences encoding a TGFβ superfamily type I receptor polypeptide, type II receptor polypeptide, and/or co-receptor polypeptide. Such useful expression control sequences, include, for example, the early and late promoters of SV40, tet promoter, adenovirus or cytomegalovirus immediate early promoter, RSV promoters, the lac system, the trp system, the TAC or TRC system, T7 promoter whose expression is directed by T7 RNA polymerase, the major operator and promoter regions of phage lambda, the control regions for fd coat protein, the promoter for 3-phosphoglycerate kinase or other glycolytic enzymes, the promoters of acid phosphatase, e.g., Pho5, the promoters of the yeast α-mating factors, the polyhedron promoter of the baculovirus system and other sequences known to control the expression of genes of prokaryotic or eukaryotic cells or their viruses, and various combinations thereof. It should be understood that the design of the expression vector may depend on such factors as the choice of the host cell to be transformed and/or the type of protein desired to be expressed. Moreover, the vector's copy number, the ability to control that copy number and the expression of any other protein encoded by the vector, such as antibiotic markers, should also be considered.

A recombinant nucleic acid of the present disclosure can be produced by ligating the cloned gene, or a portion thereof, into a vector suitable for expression in either prokaryotic cells, eukaryotic cells (yeast, avian, insect or mammalian), or both. Expression vehicles for production of a recombinant TGFβ superfamily type I receptor polypeptide, type II receptor polypeptide, and/or co-receptor polypeptide include plasmids and other vectors. For instance, suitable vectors include plasmids of the following types: pBR322-derived plasmids, pEMBL-derived plasmids, pEX-derived plasmids, pBTac-derived plasmids and pUC-derived plasmids for expression in prokaryotic cells, such as E. coli.

Some mammalian expression vectors contain both prokaryotic sequences to facilitate the propagation of the vector in bacteria, and one or more eukaryotic transcription units that are expressed in eukaryotic cells. The pcDNAI/amp, pcDNAI/neo, pRc/CMV, pSV2gpt, pSV2neo, pSV2-dhfr, pTk2, pRSVneo, pMSG, pSVT7, pko-neo and pHyg derived vectors are examples of mammalian expression vectors suitable for transfection of eukaryotic cells. Some of these vectors are modified with sequences from bacterial plasmids, such as pBR322, to facilitate replication and drug resistance selection in both prokaryotic and eukaryotic cells. Alternatively, derivatives of viruses such as the bovine papilloma virus (BPV-1), or Epstein-Barr virus (pHEBo, pREP-derived and p205) can be used for transient expression of proteins in eukaryotic cells. Examples of other viral (including retroviral) expression systems can be found below in the description of gene therapy delivery systems. The various methods employed in the preparation of the plasmids and in transformation of host organisms are well known in the art. For other suitable expression systems for both prokaryotic and eukaryotic cells, as well as general recombinant procedures, see, e.g., Molecular Cloning A Laboratory Manual, 3rd Ed., ed. by Sambrook, Fritsch and Maniatis (Cold Spring Harbor Laboratory Press, 2001). In some instances, it may be desirable to express the recombinant polypeptides by the use of a baculovirus expression system. Examples of such baculovirus expression systems include pVL-derived vectors (such as pVL1392, pVL1393 and pVL941), pAcUW-derived vectors (such as pAcUW1), and pBlueBac-derived vectors (such as the β-gal containing pBlueBac III).

In a preferred embodiment, a vector will be designed for production of the subject TGFβ superfamily type I receptor polypeptides, type II receptor polypeptides, and/or co-receptor polypeptides in CHO cells, such as a Pcmv-Script vector (Stratagene, La Jolla, Calif.), pcDNA4 vectors (Invitrogen, Carlsbad, Calif.) and pCI-neo vectors (Promega, Madison, Wis.). As will be apparent, the subject gene constructs can be used to cause expression of the subject TGFβ superfamily type I receptor polypeptides, type II receptor polypeptides, and/or co-receptor polypeptides in cells propagated in culture, e.g., to produce proteins, including fusion proteins or variant proteins, for purification.

This disclosure also pertains to a host cell transfected with a recombinant gene including a coding sequence for one or more of the subject TGFβ superfamily type I receptor polypeptides, type II receptor polypeptides, and/or co-receptor polypeptides. The host cell may be any prokaryotic or eukaryotic cell. For example, a TGFβ superfamily type I receptor polypeptide, type II receptor polypeptide, and/or co-receptor polypeptide of the disclosure may be expressed in bacterial cells such as E. coli, insect cells (e.g., using a baculovirus expression system), yeast, or mammalian cells [e.g. a Chinese hamster ovary (CHO) cell line]. Other suitable host cells are known to those skilled in the art.

Accordingly, the present disclosure further pertains to methods of producing the subject TGFβ superfamily type I receptor polypeptides, type II receptor polypeptides, and/or co-receptor polypeptides. For example, a host cell transfected with an expression vector encoding a TGFβ superfamily type I receptor polypeptide, type II receptor polypeptide, and/or co-receptor polypeptide can be cultured under appropriate conditions to allow expression of the TGFβ superfamily type I receptor polypeptide, type II receptor polypeptide, and/or co-receptor polypeptide to occur. The polypeptide may be secreted and isolated from a mixture of cells and medium containing the polypeptide. Alternatively, the TGFβ superfamily type I receptor polypeptide, type II receptor polypeptide, and/or co-receptor polypeptide may be isolated from a cytoplasmic or membrane fraction obtained from harvested and lysed cells. A cell culture includes host cells, media and other byproducts. Suitable media for cell culture are well known in the art. The subject polypeptides can be isolated from cell culture medium, host cells, or both, using techniques known in the art for purifying proteins, including ion-exchange chromatography, gel filtration chromatography, ultrafiltration, electrophoresis, immunoaffinity purification with antibodies specific for particular epitopes of the TGFβ superfamily type I receptor polypeptides, type II receptor polypeptides, and/or co-receptor polypeptides and affinity purification with an agent that binds to a domain fused to TGFβ superfamily type I receptor polypeptides, type II receptor polypeptides, and/or co-receptor polypeptides (e.g., a protein A column may be used to purify a TGFβ superfamily type I receptor-Fc fusion protein, type II receptor-Fc fusion protein, and/or co-receptor-Fc fusion protein). In some embodiments, the TGFβ superfamily type I receptor polypeptide, type II receptor polypeptide, and/or co-receptor polypeptide is a fusion protein containing a domain which facilitates its purification.

In some embodiments, purification is achieved by a series of column chromatography steps, including, for example, three or more of the following, in any order: protein A chromatography, Q sepharose chromatography, phenylsepharose chromatography, size exclusion chromatography, and cation exchange chromatography. The purification could be completed with viral filtration and buffer exchange. A TGFβ superfamily type I receptor-Fc fusion protein, type II receptor-Fc fusion protein, and/or co-receptor-Fc fusion protein may be purified to a purity of >90%, >95%, >96%, >98%, or >99% as determined by size exclusion chromatography and >90%, >95%, >96%, >98%, or >99% as determined by SDS PAGE. The target level of purity should be one that is sufficient to achieve desirable results in mammalian systems, particularly non-human primates, rodents (mice), and humans.

In another embodiment, a fusion gene coding for a purification leader sequence, such as a poly-(His)/enterokinase cleavage site sequence at the N-terminus of the desired portion of the recombinant TGFβ superfamily type I receptor polypeptide, type II receptor polypeptide, and/or co-receptor polypeptide, can allow purification of the expressed fusion protein by affinity chromatography using a Ni²⁺ metal resin. The purification leader sequence can then be subsequently removed by treatment with enterokinase to provide the purified TGFβ superfamily type I receptor polypeptide, type II receptor polypeptide, and/or co-receptor polypeptide. See, e.g., Hochuli et al. (1987) J. Chromatography 411:177; and Janknecht et al. (1991) PNAS USA 88:8972.

Techniques for making fusion genes are well known. Essentially, the joining of various DNA fragments coding for different polypeptide sequences is performed in accordance with conventional techniques, employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed to generate a chimeric gene sequence. See, e.g., Current Protocols in Molecular Biology, eds. Ausubel et al., John Wiley & Sons: 1992.

### 4. Screening Assays

In certain aspects, the present disclosure relates to the use of TGFβ superfamily heteromultimers (e.g., a TGFβ superfamily co-receptor heteromultimer) to identify compounds (agents) which are agonists or antagonists of TGFβ superfamily receptors. Compounds identified through this screening can be tested to assess their ability to modulate tissues such as bone, cartilage, muscle, fat, and/or neurons, to assess their ability to modulate tissue growth in vivo or in vitro. These compounds can be tested, for example, in animal models.

There are numerous approaches to screening for therapeutic agents for modulating tissue growth by targeting TGFβ superfamily ligand signaling (e.g., SMAD signaling). In certain embodiments, high-throughput screening of compounds can be carried out to identify agents that perturb TGFβ superfamily receptor-mediated effects on a selected cell line. In certain embodiments, the assay is carried out to screen and identify compounds that specifically inhibit or reduce binding of a TGF-beta superfamily heteromultimer to its binding partner, such as a TGFβ superfamily ligand (e.g., BMP2, BMP2/7, BMP3, BMP4, BMP4/7, BMP5, BMP6, BMP7, BMP8a, BMP8b, BMP9, BMP10, GDF3, GDF5, GDF6/BMP13, GDF7, GDF8, GDF9b/BMP15, GDF11/BMP11, GDF15/MIC1, TGF-β1, TGF-β2, TGF-β3, activin A, activin B, activin C, activin E, activin AB, activin AC, activin AE, activin BC, activin BE, nodal, glial cell-derived neurotrophic factor (GDNF), neurturin, artemin, persephin, MIS, and Lefty). Alternatively, the assay can be used to identify compounds that enhance binding of a TGF-beta superfamily heteromultimer to its binding partner such as a TGFβ superfamily ligand. In a further embodiment, the compounds can be identified by their ability to interact with a TGF-beta superfamily heteromultimer of the disclosure.

A variety of assay formats will suffice and, in light of the present disclosure, those not expressly described herein will nevertheless be comprehended by one of ordinary skill in the art. As described herein, the test compounds (agents) of the invention may be created by any combinatorial chemical method. Alternatively, the subject compounds may be naturally occurring biomolecules synthesized in vivo or in vitro. Compounds (agents) to be tested for their ability to act as modulators of tissue growth can be produced, for example, by bacteria, yeast, plants or other organisms (e.g., natural products), produced chemically (e.g., small molecules, including peptidomimetics), or produced recombinantly. Test compounds contemplated by the present invention include non-peptidyl organic molecules, peptides, polypeptides, peptidomimetics, sugars, hormones, and nucleic acid molecules. In certain embodiments, the test agent is a small organic molecule having a molecular weight of less than about 2,000 Daltons.

The test compounds of the disclosure can be provided as single, discrete entities, or provided in libraries of greater complexity, such as made by combinatorial chemistry. These libraries can comprise, for example, alcohols, alkyl halides, amines, amides, esters, aldehydes, ethers and other classes of organic compounds. Presentation of test compounds to the test system can be in either an isolated form or as mixtures of compounds, especially in initial screening steps. Optionally, the compounds may be optionally derivatized with other compounds and have derivatizing groups that facilitate isolation of the compounds. Non-limiting examples of derivatizing groups include biotin, fluorescein, digoxygenin, green fluorescent protein, isotopes, polyhistidine, magnetic beads, glutathione S-transferase (GST), photoactivatable crosslinkers or any combinations thereof.

In many drug-screening programs which test libraries of compounds and natural extracts, high-throughput assays are desirable in order to maximize the number of compounds surveyed in a given period of time. Assays which are performed in cell-free systems, such as may be derived with purified or semi-purified proteins, are often preferred as “primary” screens in that they can be generated to permit rapid development and relatively easy detection of an alteration in a molecular target which is mediated by a test compound. Moreover, the effects of cellular toxicity or bioavailability of the test compound can be generally ignored in the in vitro system, the assay instead being focused primarily on the effect of the drug on the molecular target as may be manifest in an alteration of binding affinity between a TGF-beta superfamily heteromultimer and its binding partner (e.g., BMP2, BMP2/7, BMP3, BMP4, BMP4/7, BMP5, BMP6, BMP7, BMP8a, BMP8b, BMP9, BMP10, GDF3, GDF5, GDF6/BMP13, GDF7, GDF8, GDF9b/BMP15, GDF11/BMP11, GDF15/MIC1, TGF-β1, TGF-β2, TGF-β3, activin A, activin B, activin C, activin E, activin AB, activin AC, activin AE, activin BC, activin BE, nodal, glial cell-derived neurotrophic factor (GDNF), neurturin, artemin, persephin, MIS, and Lefty).

Merely to illustrate, in an exemplary screening assay of the present disclosure, the compound of interest is contacted with an isolated and purified TGF-beta superfamily heteromultimercomplex which is ordinarily capable of binding to a TGF-beta superfamily ligand, as appropriate for the intention of the assay. To the mixture of the compound and TGF-beta superfamily heteromultimer is then added to a composition containing the appropriate TGF-beta superfamily ligand (e.g., BMP2, BMP2/7, BMP3, BMP4, BMP4/7, BMP5, BMP6, BMP7, BMP8a, BMP8b, BMP9, BMP10, GDF3, GDF5, GDF6/BMP13, GDF7, GDF8, GDF9b/BMP15, GDF11/BMP11, GDF15/MIC1, TGF-β1, TGF-β2, TGF-β3, activin A, activin B, activin C, activin E, activin AB, activin AC, activin AE, activin BC, activin BE, nodal, glial cell-derived neurotrophic factor (GDNF), neurturin, artemin, persephin, MIS, and Lefty). Detection and quantification of heteromultimer-superfamily ligand complexes provides a means for determining the compound's efficacy at inhibiting (or potentiating) complex formation between the TGF-beta superfamily heteromultimer complex and its binding protein. The efficacy of the compound can be assessed by generating dose-response curves from data obtained using various concentrations of the test compound. Moreover, a control assay can also be performed to provide a baseline for comparison. For example, in a control assay, isolated and purified TGF-beta superfamily ligand is added to a composition containing the TGF-beta superfamily heteromultimer, and the formation of heteromultimer-ligand complex is quantitated in the absence of the test compound. It will be understood that, in general, the order in which the reactants may be admixed can be varied, and can be admixed simultaneously. Moreover, in place of purified proteins, cellular extracts and lysates may be used to render a suitable cell-free assay system.

Binding of a TGF-beta superfamily heteromultimer to another protein may be detected by a variety of techniques. For instance, modulation of the formation of complexes can be quantitated using, for example, detectably labeled proteins such as radiolabeled (e.g., ³²P, ³⁵S, ¹⁴C or ³H), fluorescently labeled (e.g., FITC), or enzymatically labeled TGF-beta superfamily heteromultimer and/or its binding protein, by immunoassay, or by chromatographic detection.

In certain embodiments, the present disclosure contemplates the use of fluorescence polarization assays and fluorescence resonance energy transfer (FRET) assays in measuring, either directly or indirectly, the degree of interaction between a TGF-beta superfamily heteromultimer and its binding protein. Further, other modes of detection, such as those based on optical waveguides (see, e.g., PCT Publication WO 96/26432 and U.S. Pat. No. 5,677,196), surface plasmon resonance (SPR), surface charge sensors, and surface force sensors, are compatible with many embodiments of the disclosure.

Moreover, the present disclosure contemplates the use of an interaction trap assay, also known as the “two-hybrid assay,” for identifying agents that disrupt or potentiate interaction between a TGF-beta superfamily heteromultimer and its binding partner. See, e.g., U.S. Pat. No. 5,283,317; Zervos et al. (1993) Cell 72:223-232; Madura et al. (1993) J Biol Chem 268:12046-12054; Bartel et al. (1993) Biotechniques 14:920-924; and Iwabuchi et al. (1993) Oncogene 8:1693-1696). In a specific embodiment, the present disclosure contemplates the use of reverse two-hybrid systems to identify compounds (e.g., small molecules or peptides) that dissociate interactions between a TGF-beta superfamily heteromultimer and its binding protein [see, e.g., Vidal and Legrain, (1999) Nucleic Acids Res 27:919-29; Vidal and Legrain, (1999) Trends Biotechnol 17:374-81; and U.S. Pat. Nos. 5,525,490; 5,955,280; and 5,965,368].

In certain embodiments, the subject compounds are identified by their ability to interact with a TGF-beta superfamily heteromultimer of the disclosure. The interaction between the compound and the TGF-beta superfamily heteromultimer may be covalent or non-covalent. For example, such interaction can be identified at the protein level using in vitro biochemical methods, including photo-crosslinking, radiolabeled ligand binding, and affinity chromatography. See, e.g., Jakoby W B et al. (1974) Methods in Enzymology 46:1. In certain cases, the compounds may be screened in a mechanism-based assay, such as an assay to detect compounds which bind to a TGF-beta superfamily heteromultimer. This may include a solid-phase or fluid-phase binding event. Alternatively, the gene encoding a TGF-beta superfamily heteromultimer can be transfected with a reporter system (e.g., β-galactosidase, luciferase, or green fluorescent protein) into a cell and screened against the library preferably by high-throughput screening or with individual members of the library. Other mechanism-based binding assays may be used; for example, binding assays which detect changes in free energy. Binding assays can be performed with the target fixed to a well, bead or chip or captured by an immobilized antibody or resolved by capillary electrophoresis. The bound compounds may be detected usually using colorimetric endpoints or fluorescence or surface plasmon resonance.

### 5. Exemplary Therapeutic Uses

In aspects embodiments, a TGF-beta superfamily heteromultimer, or combination of TGF-beta superfamily heteromultimers, of the present disclosure can be administered to a patient in need thereof. In some embodiments, the present invention provides methods of treating a disorder or condition in a patient in need thereof by administering to the patient a therapeutically effective amount of a TGF-beta superfamily heteromultimer, or combination of TGF-beta superfamily heteromultimers, as described herein. In some embodiments, the present invention provides methods of preventing a disorder or condition in a patient in need thereof by administering to the patient a therapeutically effective amount of a TGF-beta superfamily heteromultimer, or combination of TGF-beta superfamily heteromultimers, as described herein. In some embodiments, the present invention provides methods of delaying the progression or onset a disorder or condition in a patient in need thereof by administering to the patient a therapeutically effective amount of a TGF-beta superfamily heteromultimer, or combination of TGF-beta superfamily heteromultimers, as described herein. In some embodiments, the present invention provides methods of treating one or more complications of a disorder or condition in a patient in need thereof by administering to the patient a therapeutically effective amount of a TGF-beta superfamily heteromultimer, or combination of TGF-beta superfamily heteromultimers, as described herein. In some embodiments, the disorder or condition is one or more of: anemia, a thalassemia, myelodysplastic syndrome (MDS), sickle cell disease, and a bone-related disorder (e.g., a bone-related disorder associated with one or more of low bone density, low bone strength, and/or low bone growth). In some embodiments, the methods of the disclosure relate to increasing bone growth in a patient in need thereof. In some embodiments, the methods of the disclosure relate to increasing bone strength in a patient in need thereof. In some embodiments, the methods of the disclosure relate to increasing bone density (e.g., bone mineral density) in a patient in need thereof. In some embodiments, the methods of the disclosure relate to increasing red blood cell levels in a patient in need thereof. In some embodiments, the methods of the disclosure relate to increasing hemoglobin levels in a patient in need thereof. Optionally, any of the TGF-beta superfamily heteromultimers of the present disclosure can potentially be employed individually or in combination for therapeutic uses disclosed herein. These methods are particularly aimed at therapeutic and prophylactic treatments of mammals including, for example, rodents, primates, and humans.

As used herein, a therapeutic that “prevents” a disorder or condition refers to a compound that, in a statistical sample, reduces the occurrence of the disorder or condition in the treated sample relative to an untreated control sample, or delays the onset or reduces the severity of one or more symptoms of the disorder or condition relative to the untreated control sample. The term “treating” as used herein includes amelioration or elimination of the condition once it has been established. In either case, prevention or treatment may be discerned in the diagnosis provided by a physician or other health care provider and the intended result of administration of the therapeutic agent.

In certain embodiments, a TGF-beta superfamily heteromultimer, or combinations of TGF-beta superfamily heteromultimers, of the present disclosure may be used in methods of inducing bone and/or cartilage formation, preventing bone loss, increasing bone mineralization, preventing the demineralization of bone, and/or increasing bone density. TGF-beta superfamily heteromultimers may be useful in patients who are diagnosed with subclinical low bone density, as a protective measure against the development of osteoporosis.

In some embodiments, a TGF-beta superfamily heteromultimer, or combinations of TGF-beta superfamily heteromultimers, of the present disclosure may find medical utility in the healing of bone fractures and cartilage defects in humans and other animals. The subject methods and compositions may also have prophylactic use in closed as well as open fracture reduction and also in the improved fixation of artificial joints. De novo bone formation induced by an osteogenic agent is useful for repair of craniofacial defects that are congenital, trauma-induced, or caused by oncologic resection, and is also useful in cosmetic plastic surgery. Further, methods and compositions of the invention may be used in the treatment of periodontal disease and in other tooth repair processes. In certain cases, a TGF-beta superfamily heteromultimer, or combinations of TGF-beta superfamily heteromultimers, may provide an environment to attract bone-forming cells, stimulate growth of bone-forming cells, or induce differentiation of progenitors of bone-forming cells. TGF-beta superfamily heteromultimers of the disclosure may also be useful in the treatment of osteoporosis. Further, TGF-beta superfamily heteromultimers may be used in repair of cartilage defects and prevention/reversal of osteoarthritis.

In some embodiments, methods and compositions of the disclosure can be applied to conditions characterized by or causing bone loss, such as osteoporosis (including secondary osteoporosis), hyperparathyroidism, mineral bone disorder, sex hormone deprivation or ablation (e.g. androgen and/or estrogen), glucocorticoid treatment, rheumatoid arthritis, severe burns, hyperparathyroidism, hypercalcemia, hypocalcemia, hypophosphatemia, osteomalacia (including tumor-induced osteomalacia), hyperphosphatemia, vitamin D deficiency, hyperparathyroidism (including familial hyperparathyroidism) and pseudohypoparathyroidism, tumor metastases to bone, bone loss as a consequence of a tumor or chemotherapy, tumors of the bone and bone marrow (e.g., multiple myeloma), ischemic bone disorders, periodontal disease and oral bone loss, Cushing's disease, Paget's disease, thyrotoxicosis, chronic diarrheal state or malabsorption, renal tubular acidosis, or anorexia nervosa. Methods and compositions of the invention may also be applied to conditions characterized by a failure of bone formation or healing, including non-union fractures, fractures that are otherwise slow to heal, fetal and neonatal bone dysplasias (e.g., hypocalcemia, hypercalcemia, calcium receptor defects and vitamin D deficiency), osteonecrosis (including osteonecrosis of the jaw) and osteogenesis imperfecta. Additionally, the anabolic effects will cause such antagonists to diminish bone pain associated with bone damage or erosion. As a consequence of the anti-resorptive effects, such antagonists may be useful to treat disorders of abnormal bone formation, such as osteoblastic tumor metastases (e.g., associated with primary prostate or breast cancer), osteogenic osteosarcoma, osteopetrosis, progressive diaphyseal dysplasia, endosteal hyperostosis, osteopoikilosis, and melorheostosis. Other disorders that may be treated include fibrous dysplasia and chondrodysplasias.

In another specific embodiment, the disclosure provides a therapeutic method and composition for repairing fractures and other conditions related to cartilage and/or bone defects or periodontal diseases. The invention further provides therapeutic methods and compositions for wound healing and tissue repair. The types of wounds include, but are not limited to, burns, incisions and ulcers. See, e.g., PCT Publication No. WO 84/01106. Such compositions comprise a therapeutically effective amount of at least one of the TGF-beta superfamily heteromultimers of the disclosure in admixture with a pharmaceutically acceptable vehicle, carrier, or matrix.

In some embodiments, a TGF-beta superfamily heteromultimer, or combinations of TGF-beta superfamily heteromultimers, of the disclosure can be applied to conditions causing bone loss such as osteoporosis, hyperparathyroidism, Cushing's disease, thyrotoxicosis, chronic diarrheal state or malabsorption, renal tubular acidosis, or anorexia nervosa. It is commonly appreciated that being female, having a low body weight, and leading a sedentary lifestyle are risk factors for osteoporosis (loss of bone mineral density, leading to fracture risk). However, osteoporosis can also result from the long-term use of certain medications. Osteoporosis resulting from drugs or another medical condition is known as secondary osteoporosis. In Cushing's disease, the excess amount of cortisol produced by the body results in osteoporosis and fractures. The most common medications associated with secondary osteoporosis are the corticosteroids, a class of drugs that act like cortisol, a hormone produced naturally by the adrenal glands. Although adequate levels of thyroid hormones are needed for the development of the skeleton, excess thyroid hormone can decrease bone mass over time. Antacids that contain aluminum can lead to bone loss when taken in high doses. Other medications that can cause secondary osteoporosis include phenytoin (Dilantin) and barbiturates that are used to prevent seizures; methotrexate (Rheumatrex, Immunex, Folex PFS), a drug for some forms of arthritis, cancer, and immune disorders; cyclosporine (Sandimmune, Neoral), a drug used to treat some autoimmune diseases and to suppress the immune system in organ transplant patients; luteinizing hormone-releasing hormone agonists (Lupron, Zoladex), used to treat prostate cancer and endometriosis; heparin (Calciparine, Liquaemin), an anticlotting medication; and cholestyramine (Questran) and colestipol (Colestid), used to treat high cholesterol. Bone loss resulting from cancer therapy is widely recognized and termed cancer therapy-induced bone loss (CTIBL). Bone metastases can create cavities in the bone that may be corrected by treatment with a TGF-beta superfamily heteromultimer. Bone loss can also be caused by gum disease, a chronic infection in which bacteria located in gum recesses produce toxins and harmful enzymes.

In a further embodiment, the present disclosure provides methods and therapeutic agents for treating diseases or disorders associated with abnormal or unwanted bone growth. For example, patients with the congenital disorder fibrodysplasia ossificans progressiva (FOP) are afflicted by progressive ectopic bone growth in soft tissues spontaneously or in response to tissue trauma, with a major impact on quality of life. Additionally, abnormal bone growth can occur after hip replacement surgery and thus ruin the surgical outcome. This is a more common example of pathological bone growth and a situation in which the subject methods and compositions may be therapeutically useful. The same methods and compositions may also be useful for treating other forms of abnormal bone growth (e.g., pathological growth of bone following trauma, burns or spinal cord injury), and for treating or preventing the undesirable conditions associated with the abnormal bone growth seen in connection with metastatic prostate cancer or osteosarcoma.

In certain embodiments, a TGF-beta superfamily heteromultimer, or combinations of TGF-beta superfamily heteromultimers, of the disclosure may be used to promote bone formation in patients with cancer. Patients having certain tumors are at high risk for bone loss due to tumor-induced bone loss, bone metastases, and therapeutic agents. Generally, DEXA scans are employed to assess changes in bone density, while indicators of bone remodeling may be used to assess the likelihood of bone metastases. Serum markers may be monitored. Bone specific alkaline phosphatase (BSAP) is an enzyme that is present in osteoblasts. Blood levels of BSAP are increased in patients with bone metastasis and other conditions that result in increased bone remodeling. Osteocalcin and procollagen peptides are also associated with bone formation and bone metastases. Increases in BSAP have been detected in patients with bone metastasis caused by prostate cancer, and to a lesser degree, in bone metastases from breast cancer. BMP7 levels are high in prostate cancer that has metastasized to bone, but not in bone metastases due to bladder, skin, liver, or lung cancer. Type I carboxy-terminal telopeptide (ICTP) is a crosslink found in collagen that is formed during to the resorption of bone. Since bone is constantly being broken down and reformed, ICTP will be found throughout the body. However, at the site of bone metastasis, the level will be significantly higher than in an area of normal bone. ICTP has been found in high levels in bone metastasis due to prostate, lung, and breast cancer. Another collagen crosslink, Type I N-terminal telopeptide (NTx), is produced along with ICTP during bone turnover. The amount of NTx is increased in bone metastasis caused by many different types of cancer including lung, prostate, and breast cancer. Also, the levels of NTx increase with the progression of the bone metastasis. Therefore, this marker can be used to both detect metastasis as well as measure the extent of the disease. Other markers of resorption include pyridinoline and deoxypyridinoline. Any increase in resorption markers or markers of bone metastases indicate the need for therapy with a TGF-beta superfamily heteromultimer, or combinations of TGF-beta superfamily heteromultimers, in a patient.

A TGF-beta superfamily heteromultimer, or combinations of TGF-beta superfamily heteromultimers, of the disclosure may be conjointly administered with other bone-active pharmaceutical agents. Conjoint administration may be accomplished by administration of a single co-formulation, by simultaneous administration, or by administration at separate times. TGF-beta superfamily heteromultimer complexes may be particularly advantageous if administered with other bone-active agents. A patient may benefit from conjointly receiving a TGF-beta superfamily heteromultimer complex and taking calcium supplements, vitamin D, appropriate exercise and/or, in some cases, other medication. Examples of other medications include, bisphosphonates (alendronate, ibandronate and risedronate), calcitonin, estrogens, parathyroid hormone and raloxifene. The bisphosphonates (alendronate, ibandronate and risedronate), calcitonin, estrogens and raloxifene affect the bone remodeling cycle and are classified as anti-resorptive medications. Bone remodeling consists of two distinct stages: bone resorption and bone formation. Anti-resorptive medications slow or stop the bone-resorbing portion of the bone-remodeling cycle but do not slow the bone-forming portion of the cycle. As a result, new formation continues at a greater rate than bone resorption, and bone density may increase over time. Teriparatide, a form of parathyroid hormone, increases the rate of bone formation in the bone remodeling cycle. Alendronate is approved for both the prevention (5 mg per day or 35 mg once a week) and treatment (10 mg per day or 70 mg once a week) of postmenopausal osteoporosis. Alendronate reduces bone loss, increases bone density and reduces the risk of spine, wrist and hip fractures. Alendronate also is approved for treatment of glucocorticoid-induced osteoporosis in men and women as a result of long-term use of these medications (i.e., prednisone and cortisone) and for the treatment of osteoporosis in men. Alendronate plus vitamin D is approved for the treatment of osteoporosis in postmenopausal women (70 mg once a week plus vitamin D), and for treatment to improve bone mass in men with osteoporosis. Ibandronate is approved for the prevention and treatment of postmenopausal osteoporosis. Taken as a once-a-month pill (150 mg), ibandronate should be taken on the same day each month. Ibandronate reduces bone loss, increases bone density and reduces the risk of spine fractures. Risedronate is approved for the prevention and treatment of postmenopausal osteoporosis. Taken daily (5 mg dose) or weekly (35 mg dose or 35 mg dose with calcium), risedronate slows bone loss, increases bone density and reduces the risk of spine and non-spine fractures. Risedronate also is approved for use by men and women to prevent and/or treat glucocorticoid-induced osteoporosis that results from long-term use of these medications (i.e., prednisone or cortisone). Calcitonin is a naturally occurring hormone involved in calcium regulation and bone metabolism. In women who are more than 5 years beyond menopause, calcitonin slows bone loss, increases spinal bone density, and may relieve the pain associated with bone fractures. Calcitonin reduces the risk of spinal fractures. Calcitonin is available as an injection (50-100 IU daily) or nasal spray (200 IU daily).

A patient may also benefit from conjointly receiving a TGF-beta superfamily heteromultimer, or combinations of TGF-beta superfamily heteromultimers, and additional bone-active medications. Estrogen therapy (ET)/hormone therapy (HT) is approved for the prevention of osteoporosis. ET has been shown to reduce bone loss, increase bone density in both the spine and hip, and reduce the risk of hip and spinal fractures in postmenopausal women. ET is administered most commonly in the form of a pill or skin patch that delivers a low dose of approximately 0.3 mg daily or a standard dose of approximately 0.625 mg daily and is effective even when started after age 70. When estrogen is taken alone, it can increase a woman's risk of developing cancer of the uterine lining (endometrial cancer). To eliminate this risk, healthcare providers prescribe the hormone progestin in combination with estrogen (hormone replacement therapy or HT) for those women who have an intact uterus. ET/HT relieves menopause symptoms and has been shown to have a beneficial effect on bone health. Side effects may include vaginal bleeding, breast tenderness, mood disturbances and gallbladder disease. Raloxifene, 60 mg a day, is approved for the prevention and treatment of postmenopausal osteoporosis. It is from a class of drugs called Selective Estrogen Receptor Modulators (SERMs) that have been developed to provide the beneficial effects of estrogens without their potential disadvantages. Raloxifene increases bone mass and reduces the risk of spine fractures. Data are not yet available to demonstrate that raloxifene can reduce the risk of hip and other non-spine fractures. Teriparatide, a form of parathyroid hormone, is approved for the treatment of osteoporosis in postmenopausal women and men who are at high risk for a fracture. This medication stimulates new bone formation and significantly increases bone mineral density. In postmenopausal women, fracture reduction was noted in the spine, hip, foot, ribs and wrist. In men, fracture reduction was noted in the spine, but there were insufficient data to evaluate fracture reduction at other sites. Teriparatide is self-administered as a daily injection for up to 24 months.

In certain aspects, a TGF-beta superfamily heteromultimer, or combinations of TGF-beta superfamily heteromultimers, of the present disclosure can be used to increase red blood cell levels, treat or prevent an anemia, and/or treat or prevent ineffective erythropoiesis in a subject in need thereof. In certain aspects, a TGF-beta superfamily heteromultimer, or combinations of TGF-beta superfamily heteromultimers, of the present disclosure may be used in combination with conventional therapeutic approaches for increasing red blood cell levels, particularly those used to treat anemias of multifactorial origin. Conventional therapeutic approaches for increasing red blood cell levels include, for example, red blood cell transfusion, administration of one or more EPO receptor activators, hematopoietic stem cell transplantation, immunosuppressive biologics and drugs (e.g., corticosteroids). In certain embodiments, a TGF-beta superfamily heteromultimer, or combinations of TGF-beta superfamily heteromultimers, of the present disclosure can be used to treat or prevent ineffective erythropoiesis and/or the disorders associated with ineffective erythropoiesis in a subject in need thereof. In certain aspects, a TGF-beta superfamily heteromultimer, or combinations of TGF-beta superfamily heteromultimers, of the present disclosure can be used in combination with conventional therapeutic approaches for treating or preventing an anemia or ineffective erythropoiesis disorder, particularly those used to treat anemias of multifactorial origin.

In certain embodiments, a TGF-beta superfamily heteromultimer, or combinations of TGF-beta superfamily heteromultimers, optionally combined with an EPO receptor activator, may be used to increase red blood cell, hemoglobin, or reticulocyte levels in healthy individuals and selected patient populations. Examples of appropriate patient populations include those with undesirably low red blood cell or hemoglobin levels, such as patients having an anemia, and those that are at risk for developing undesirably low red blood cell or hemoglobin levels, such as those patients who are about to undergo major surgery or other procedures that may result in substantial blood loss. In one embodiment, a patient with adequate red blood cell levels is treated with a TGF-beta superfamily heteromultimer, or combinations of TGF-beta superfamily heteromultimers, to increase red blood cell levels, and then blood is drawn and stored for later use in transfusions.

One or more TGF-beta superfamily heteromultimers of the disclosure, optionally combined with an EPO receptor activator, may be used to increase red blood cell levels, hemoglobin levels, and/or hematocrit levels in a patient having an anemia. When observing hemoglobin and/or hematocrit levels in humans, a level of less than normal for the appropriate age and gender category may be indicative of anemia, although individual variations are taken into account. For example, a hemoglobin level from 10-12.5 g/dl, and typically about 11.0 g/dl is considered to be within the normal range in health adults, although, in terms of therapy, a lower target level may cause fewer cardiovascular side effects [see, e.g., Jacobs et al. (2000) Nephrol Dial Transplant 15, 15-19]. Alternatively, hematocrit levels (percentage of the volume of a blood sample occupied by the cells) can be used as a measure for anemia. Hematocrit levels for healthy individuals range from about 41-51% for adult males and from 35-45% for adult females. In certain embodiments, a patient may be treated with a dosing regimen intended to restore the patient to a target level of red blood cells, hemoglobin, and/or hematocrit. As hemoglobin and hematocrit levels vary from person to person, optimally, the target hemoglobin and/or hematocrit level can be individualized for each patient.

Anemia is frequently observed in patients having a tissue injury, an infection, and/or a chronic disease, particularly cancer. In some subjects, anemia is distinguished by low erythropoietin levels and/or an inadequate response to erythropoietin in the bone marrow [see, e.g., Adamson (2008) Harrison's Principles of Internal Medicine, 17th ed.; McGraw Hill, New York, pp 628-634]. Potential causes of anemia include, for example, blood loss, nutritional deficits (e.g. reduced dietary intake of protein), medication reaction, various problems associated with the bone marrow, and many diseases. More particularly, anemia has been associated with a variety of disorders and conditions that include, for example, bone marrow transplantation; solid tumors (e.g., breast cancer, lung cancer, and colon cancer); tumors of the lymphatic system (e.g., chronic lymphocyte leukemia, non-Hodgkins lymphoma, and Hodgkins lymphoma); tumors of the hematopoietic system (e.g., leukemia, a myelodysplastic syndrome and multiple myeloma); radiation therapy; chemotherapy (e.g., platinum containing regimens); inflammatory and autoimmune diseases, including, but not limited to, rheumatoid arthritis, other inflammatory arthritides, systemic lupus erythematosis (SLE), acute or chronic skin diseases (e.g., psoriasis), inflammatory bowel disease (e.g., Crohn's disease and ulcerative colitis); acute or chronic renal disease or failure, including idiopathic or congenital conditions; acute or chronic liver disease; acute or chronic bleeding; situations where transfusion of red blood cells is not possible due to patient allo- or auto-antibodies and/or for religious reasons (e.g., some Jehovah's Witnesses); infections (e.g., malaria and osteomyelitis); hemoglobinopathies including, for example, sickle cell disease (anemia), thalassemias; drug use or abuse (e.g., alcohol misuse); pediatric patients with anemia from any cause to avoid transfusion; and elderly patients or patients with underlying cardiopulmonary disease with anemia who cannot receive transfusions due to concerns about circulatory overload [see, e.g., Adamson (2008) Harrison's Principles of Internal Medicine, 17th ed.; McGraw Hill, New York, pp 628-634]. In some embodiments, one or more TGF-beta superfamily heteromultimers of the disclosure could be used to treat or prevent anemia associated with one or more of the disorders or conditions disclosed herein.

Many factors can contribute to cancer-related anemia. Some are associated with the disease process itself and the generation of inflammatory cytokines such as interleukin-1, interferon-gamma, and tumor necrosis factor [Bron et al. (2001) Semin Oncol 28(Suppl 8):1-6]. Among its effects, inflammation induces the key iron-regulatory peptide hepcidin, thereby inhibiting iron export from macrophages and generally limiting iron availability for erythropoiesis [see, e.g., Ganz (2007) J Am Soc Nephrol 18:394-400]. Blood loss through various routes can also contribute to cancer-related anemia. The prevalence of anemia due to cancer progression varies with cancer type, ranging from 5% in prostate cancer up to 90% in multiple myeloma. Cancer-related anemia has profound consequences for patients, including fatigue and reduced quality of life, reduced treatment efficacy, and increased mortality. In some embodiments, one or more TGF-beta superfamily heteromultimers of the disclosure, optionally combined with an EPO receptor activator, could be used to treat a cancer-related anemia.

A hypoproliferative anemia can result from primary dysfunction or failure of the bone marrow. Hypoproliferative anemias include: anemia of chronic disease, anemia associated with hypometabolic states, and anemia associated with cancer. In each of these types, endogenous erythropoietin levels are inappropriately low for the degree of anemia observed. Other hypoproliferative anemias include: early-stage iron-deficient anemia, and anemia caused by damage to the bone marrow. In these types, endogenous erythropoietin levels are appropriately elevated for the degree of anemia observed. Prominent examples would be myelosuppression caused by cancer and/or chemotherapeutic drugs or cancer radiation therapy. A broad review of clinical trials found that mild anemia can occur in 100% of patients after chemotherapy, while more severe anemia can occur in up to 80% of such patients [see, e.g., Groopman et al. (1999) J Natl Cancer Inst 91:1616-1634]. Myelosuppressive drugs include, for example: 1) alkylating agents such as nitrogen mustards (e.g., melphalan) and nitrosoureas (e.g., streptozocin); 2) antimetabolites such as folic acid antagonists (e.g., methotrexate), purine analogs (e.g., thioguanine), and pyrimidine analogs (e.g., gemcitabine); 3) cytotoxic antibiotics such as anthracyclines (e.g., doxorubicin); 4) kinase inhibitors (e.g., gefitinib); 5) mitotic inhibitors such as taxanes (e.g., paclitaxel) and vinca alkaloids (e.g., vinorelbine); 6) monoclonal antibodies (e.g., rituximab); and 7) topoisomerase inhibitors (e.g., topotecan and etoposide). In addition, conditions resulting in a hypometabolic rate can produce a mild-to-moderate hypoproliferative anemia. Among such conditions are endocrine deficiency states. For example, anemia can occur in Addison's disease, hypothyroidism, hyperparathyroidism, or males who are castrated or treated with estrogen. In some embodiments, one or more TGF-beta superfamily heteromultimers of the disclosure, optionally combined with an EPO receptor activator, could be used to treat a hyperproliferative anemia.

Anemia resulting from acute blood loss of sufficient volume, such as from trauma or postpartum hemorrhage, is known as acute post-hemorrhagic anemia. Acute blood loss initially causes hypovolemia without anemia since there is proportional depletion of RBCs along with other blood constituents. However, hypovolemia will rapidly trigger physiologic mechanisms that shift fluid from the extravascular to the vascular compartment, which results in hemodilution and anemia. If chronic, blood loss gradually depletes body iron stores and eventually leads to iron deficiency. In some embodiments, one or more TGF-beta superfamily heteromultimers of the disclosure, optionally combined with an EPO receptor activator, could be used to treat anemia resulting from acute blood loss.

Iron-deficiency anemia is the final stage in a graded progression of increasing iron deficiency which includes negative iron balance and iron-deficient erythropoiesis as intermediate stages. Iron deficiency can result from increased iron demand, decreased iron intake, or increased iron loss, as exemplified in conditions such as pregnancy, inadequate diet, intestinal malabsorption, acute or chronic inflammation, and acute or chronic blood loss. With mild-to-moderate anemia of this type, the bone marrow remains hypoproliferative, and RBC morphology is largely normal; however, even mild anemia can result in some microcytic hypochromic RBCs, and the transition to severe iron-deficient anemia is accompanied by hyperproliferation of the bone marrow and increasingly prevalent microcytic and hypochromic RBCs [see, e.g., Adamson (2008) Harrison's Principles of Internal Medicine, 17th ed.; McGraw Hill, New York, pp 628-634]. Appropriate therapy for iron-deficiency anemia depends on its cause and severity, with oral iron preparations, parenteral iron formulations, and RBC transfusion as major conventional options. In some embodiments, one or more TGF-beta superfamily heteromultimers of the disclosure, optionally combined with an EPO receptor activator, could be used to treat a chronic iron-deficiency.

Myelodysplastic syndrome (MDS) is a diverse collection of hematological conditions characterized by ineffective production of myeloid blood cells and risk of transformation to acute myelogenous leukemia. In MDS patients, blood stem cells do not mature into healthy red blood cells, white blood cells, or platelets. MDS disorders include, for example, refractory anemia, refractory anemia with ringed sideroblasts, refractory anemia with excess blasts, refractory anemia with excess blasts in transformation, refractory cytopenia with multilineage dysplasia, and myelodysplastic syndrome associated with an isolated 5q chromosome abnormality. As these disorders manifest as irreversible defects in both quantity and quality of hematopoietic cells, most MDS patients are afflicted with chronic anemia. Therefore, MDS patients eventually require blood transfusions and/or treatment with growth factors (e.g., erythropoietin or G-CSF) to increase red blood cell levels. However, many MDS patients develop side-effects due to frequency of such therapies. For example, patients who receive frequent red blood cell transfusion can exhibit tissue and organ damage from the buildup of extra iron. Accordingly, one or more TGF-beta superfamily heteromultimer complexes of the disclosure, may be used to treat patients having MDS. In certain embodiments, patients suffering from MDS may be treated using one or more TGF-beta superfamily heteromultimers of the disclosure, optionally in combination with an EPO receptor activator. In other embodiments, patients suffering from MDS may be treated using a combination of one or more TGF-beta superfamily heteromultimers of the disclosure and one or more additional therapeutic agents for treating MDS including, for example, thalidomide, lenalidomide, azacitadine, decitabine, erythropoietins, deferoxamine, antithymocyte globulin, and filgrastrim (G-CSF).

Originally distinguished from aplastic anemia, hemorrhage, or peripheral hemolysis on the basis of ferrokinetic studies [see, e.g., Ricketts et al. (1978) Clin Nucl Med 3:159-164], ineffective erythropoiesis describes a diverse group of anemias in which production of mature RBCs is less than would be expected given the number of erythroid precursors (erythroblasts) present in the bone marrow [Tanno et al. (2010) Adv Hematol 2010:358283]. In such anemias, tissue hypoxia persists despite elevated erythropoietin levels due to ineffective production of mature RBCs. A vicious cycle eventually develops in which elevated erythropoietin levels drive massive expansion of erythroblasts, potentially leading to splenomegaly (spleen enlargement) due to extramedullary erythropoiesis [see, e.g., Aizawa et al. (2003) Am J Hematol 74:68-72], erythroblast-induced bone pathology [see, e.g., Di Matteo et al. (2008) J Biol Regul Homeost Agents 22:211-216], and tissue iron overload, even in the absence of therapeutic RBC transfusions [see, e.g., Pippard et al. (1979) Lancet 2:819-821]. Thus, by boosting erythropoietic effectiveness, one or more TGF-beta superfamily heteromultimers of the present disclosure may break the aforementioned cycle and thus alleviate not only the underlying anemia but also the associated complications of elevated erythropoietin levels, splenomegaly, bone pathology, and tissue iron overload. In some embodiments, one or more TGF-beta superfamily heteromultimers of the present disclosure can be used to treat or prevent ineffective erythropoiesis, including anemia and elevated EPO levels as well as complications such as splenomegaly, erythroblast-induced bone pathology, iron overload, and their attendant pathologies. With splenomegaly, such pathologies include thoracic or abdominal pain and reticuloendothelial hyperplasia. Extramedullary hematopoiesis can occur not only in the spleen but potentially in other tissues in the form of extramedullary hematopoietic pseudotumors [see, e.g., Musallam et al. (2012) Cold Spring Harb Perspect Med 2:a013482]. With erythroblast-induced bone pathology, attendant pathologies include low bone mineral density, osteoporosis, and bone pain [see, e.g., Haidar et al. (2011) Bone 48:425-432]. With iron overload, attendant pathologies include hepcidin suppression and hyperabsorption of dietary iron [see, e.g., Musallam et al. (2012) Blood Rev 26(Suppl 1):516-519], multiple endocrinopathies and liver fibrosis/cirrhosis [see, e.g., Galanello et al. (2010) Orphanet J Rare Dis 5:11], and iron-overload cardiomyopathy [Lekawanvijit et al., 2009, Can J Cardiol 25:213-218].

The most common causes of ineffective erythropoiesis are the thalassemia syndromes, hereditary hemoglobinopathies in which imbalances in the production of intact alpha- and beta-hemoglobin chains lead to increased apoptosis during erythroblast maturation [see, e.g., Schrier (2002) Curr Opin Hematol 9:123-126]. Thalassemias are collectively among the most frequent genetic disorders worldwide, with changing epidemiologic patterns predicted to contribute to a growing public health problem in both the U.S. and globally [Vichinsky (2005) Ann NY Acad Sci 1054:18-24]. Thalassemia syndromes are named according to their severity. Thus, α-thalassemias include α-thalassemia minor (also known as α-thalassemia trait; two affected α-globin genes), hemoglobin H disease (three affected α-globin genes), and α-thalassemia major (also known as hydrops fetalis; four affected α-globin genes). β-Thalassemias include β-thalassemia minor (also known as β-thalassemia trait; one affected β-globin gene), β-thalassemia intermedia (two affected β-globin genes), hemoglobin E thalassemia (two affected β-globin genes), and β-thalassemia major (also known as Cooley's anemia; two affected β-globin genes resulting in a complete absence of β-globin protein). β-Thalassemia impacts multiple organs, is associated with considerable morbidity and mortality, and currently requires life-long care. Although life expectancy in patients with β-thalassemia has increased in recent years due to use of regular blood transfusions in combination with iron chelation, iron overload resulting both from transfusions and from excessive gastrointestinal absorption of iron can cause serious complications such as heart disease, thrombosis, hypogonadism, hypothyroidism, diabetes, osteoporosis, and osteopenia [see, e.g., Rund et al. (2005) N Engl J Med 353:1135-1146]. In certain embodiments, one or more TGF-beta superfamily heteromultimers of the disclosure, optionally combined with an EPO receptor activator, can be used to treat or prevent a thalassemia syndrome.

In some embodiments, one or more TGF-beta superfamily heteromultimers of the disclosure, optionally combined with an EPO receptor activator, can be used for treating disorders of ineffective erythropoiesis besides thalassemia syndromes. Such disorders include siderblastic anemia (inherited or acquired); dyserythropoietic anemia (types I and II); sickle cell anemia; hereditary spherocytosis; pyruvate kinase deficiency; megaloblastic anemias, potentially caused by conditions such as folate deficiency (due to congenital diseases, decreased intake, or increased requirements), cobalamin deficiency (due to congenital diseases, pernicious anemia, impaired absorption, pancreatic insufficiency, or decreased intake), certain drugs, or unexplained causes (congenital dyserythropoietic anemia, refractory megaloblastic anemia, or erythroleukemia); myelophthisic anemias including; congenital erythropoietic porphyria; and lead poisoning.

In certain embodiments, one or more TGF-beta superfamily heteromultimers of the disclosure may be used in combination with supportive therapies for ineffective erythropoiesis. Such therapies include transfusion with either red blood cells or whole blood to treat anemia. In chronic or hereditary anemias, normal mechanisms for iron homeostasis are overwhelmed by repeated transfusions, eventually leading to toxic and potentially fatal accumulation of iron in vital tissues such as heart, liver, and endocrine glands. Thus, supportive therapies for patients chronically afflicted with ineffective erythropoiesis also include treatment with one or more iron-chelating molecules to promote iron excretion in the urine and/or stool and thereby prevent, or reverse, tissue iron overload [see, e.g., Hershko (2006) Haematologica 91:1307-1312; Cao et al. (2011), Pediatr Rep 3(2):e17]. Effective iron-chelating agents should be able to selectively bind and neutralize ferric iron, the oxidized form of non-transferrin bound iron which likely accounts for most iron toxicity through catalytic production of hydroxyl radicals and oxidation products [see, e.g., Esposito et al. (2003) Blood 102:2670-2677]. These agents are structurally diverse, but all possess oxygen or nitrogen donor atoms able to form neutralizing octahedral coordination complexes with individual iron atoms in stoichiometries of 1:1 (hexadentate agents), 2:1 (tridentate), or 3:1 (bidentate) [Kalinowski et al. (2005) Pharmacol Rev 57:547-583]. In general, effective iron-chelating agents also are relatively low molecular weight (e.g., less than 700 daltons), with solubility in both water and lipids to enable access to affected tissues. Specific examples of iron-chelating molecules include deferoxamine, a hexadentate agent of bacterial origin requiring daily parenteral administration, and the orally active synthetic agents deferiprone (bidentate) and deferasirox (tridentate). Combination therapy consisting of same-day administration of two iron-chelating agents shows promise in patients unresponsive to chelation monotherapy and also in overcoming issues of poor patient compliance with dereroxamine alone [Cao et al. (2011) Pediatr Rep 3(2):e17; Galanello et al. (2010) Ann NY Acad Sci 1202:79-86].

As used herein, “combination”, “in combination with” or “conjoint administration” refers to any form of administration such that the second therapy is still effective in the body (e.g., the two compounds are simultaneously effective in the patient, which may include synergistic effects of the two compounds). Effectiveness may not correlate to measurable concentration of the agent in blood, serum, or plasma. For example, the different therapeutic compounds can be administered either in the same formulation or in separate formulations, either concomitantly or sequentially, and on different schedules. Thus, an individual who receives such treatment can benefit from a combined effect of different therapies. One or more TGF-beta superfamily heteromultimers of the disclosure can be administered concurrently with, prior to, or subsequent to, one or more other additional agents or supportive therapies. In general, each therapeutic agent will be administered at a dose and/or on a time schedule determined for that particular agent. The particular combination to employ in a regimen will take into account compatibility of the antagonist of the present disclosure with the therapy and/or the desired therapeutic effect to be achieved.

In certain embodiments, one or more TGF-beta superfamily heteromultimers of the disclosure may be used in combination with hepcidin or a hepcidin agonist for ineffective erythropoiesis. A circulating polypeptide produced mainly in the liver, hepcidin is considered a master regulator of iron metabolism by virtue of its ability to induce the degradation of ferroportin, an iron-export protein localized on absorptive enterocytes, hepatocytes, and macrophages. Broadly speaking, hepcidin reduces availability of extracellular iron, so hepcidin agonists may be beneficial in the treatment of ineffective erythropoiesis [see, e.g., Nemeth (2010) Adv Hematol 2010:750643]. This view is supported by beneficial effects of increased hepcidin expression in a mouse model of β-thalassemia [Gardenghi et al. (2010) J Clin Invest 120:4466-4477].

One or more TGF-beta superfamily heteromultimers of the disclosure, optionally combined with an EPO receptor activator, would also be appropriate for treating anemias of disordered RBC maturation, which are characterized in part by undersized (microcytic), oversized (macrocytic), misshapen, or abnormally colored (hypochromic) RBCs.

In certain embodiments, the present disclosure provides methods of treating or preventing anemia in an individual in need thereof by administering to the individual a therapeutically effective amount of one or more TGF-beta superfamily heteromultimers of the disclosure and an EPO receptor activator. In certain embodiments, one or more TGF-beta superfamily heteromultimers of the disclosure may be used in combination with EPO receptor activators to reduce the required dose of these activators in patients that are susceptible to adverse effects of EPO. These methods may be used for therapeutic and prophylactic treatments of a patient.

One or more TGF-beta superfamily heteromultimers of the disclosure may be used in combination with EPO receptor activators to achieve an increase in red blood cells, particularly at lower dose ranges of EPO receptor activators. This may be beneficial in reducing the known off-target effects and risks associated with high doses of EPO receptor activators. The primary adverse effects of EPO include, for example, an excessive increase in the hematocrit or hemoglobin levels and polycythemia. Elevated hematocrit levels can lead to hypertension (more particularly aggravation of hypertension) and vascular thrombosis. Other adverse effects of EPO which have been reported, some of which relate to hypertension, are headaches, influenza-like syndrome, obstruction of shunts, myocardial infarctions and cerebral convulsions due to thrombosis, hypertensive encephalopathy, and red cell blood cell aplasia. See, e.g., Singibarti (1994) J. Clin Investig 72(suppl 6), S36-S43; Horl et al. (2000) Nephrol Dial Transplant 15(suppl 4), 51-56; Delanty et al. (1997) Neurology 49, 686-689; and Bunn (2002) N Engl J Med 346(7), 522-523).

Provided that TGF-beta superfamily heteromultimers of the present disclosure act by a different mechanism than EPO, these antagonists may be useful for increasing red blood cell and hemoglobin levels in patients that do not respond well to EPO. For example, a TGF-beta superfamily heteromultimer of the present disclosure may be beneficial for a patient in which administration of a normal-to-increased dose of EPO (>300 IU/kg/week) does not result in the increase of hemoglobin level up to the target level. Patients with an inadequate EPO response are found in all types of anemia, but higher numbers of non-responders have been observed particularly frequently in patients with cancers and patients with end-stage renal disease. An inadequate response to EPO can be either constitutive (observed upon the first treatment with EPO) or acquired (observed upon repeated treatment with EPO).

In certain embodiments, the present disclosure provides methods for managing a patient that has been treated with, or is a candidate to be treated with, one or more TGF-beta superfamily heteromultimers of the disclosure by measuring one or more hematologic parameters in the patient. The hematologic parameters may be used to evaluate appropriate dosing for a patient who is a candidate to be treated with the antagonist of the present disclosure, to monitor the hematologic parameters during treatment, to evaluate whether to adjust the dosage during treatment with one or more antagonist of the disclosure, and/or to evaluate an appropriate maintenance dose of one or more antagonists of the disclosure. If one or more of the hematologic parameters are outside the normal level, dosing with one or more TGF-beta superfamily heteromultimers of the disclosure may be reduced, delayed or terminated.

Hematologic parameters that may be measured in accordance with the methods provided herein include, for example, red blood cell levels, blood pressure, iron stores, and other agents found in bodily fluids that correlate with increased red blood cell levels, using art-recognized methods. Such parameters may be determined using a blood sample from a patient. Increases in red blood cell levels, hemoglobin levels, and/or hematocrit levels may cause increases in blood pressure.

In one embodiment, if one or more hematologic parameters are outside the normal range or on the high side of normal in a patient who is a candidate to be treated with one or more TGF-beta superfamily heteromultimers of the disclosure, then onset of administration of the one or more TGF-beta superfamily heteromultimers of the disclosure may be delayed until the hematologic parameters have returned to a normal or acceptable level either naturally or via therapeutic intervention. For example, if a candidate patient is hypertensive or pre-hypertensive, then the patient may be treated with a blood pressure lowering agent in order to reduce the patient's blood pressure. Any blood pressure lowering agent appropriate for the individual patient's condition may be used including, for example, diuretics, adrenergic inhibitors (including alpha blockers and beta blockers), vasodilators, calcium channel blockers, angiotensin-converting enzyme (ACE) inhibitors, or angiotensin II receptor blockers. Blood pressure may alternatively be treated using a diet and exercise regimen. Similarly, if a candidate patient has iron stores that are lower than normal, or on the low side of normal, then the patient may be treated with an appropriate regimen of diet and/or iron supplements until the patient's iron stores have returned to a normal or acceptable level. For patients having higher than normal red blood cell levels and/or hemoglobin levels, then administration of the one or more TGF-beta superfamily heteromultimers of the disclosure may be delayed until the levels have returned to a normal or acceptable level.

In certain embodiments, if one or more hematologic parameters are outside the normal range or on the high side of normal in a patient who is a candidate to be treated with one or more TGF-beta superfamily heteromultimers of the disclosure, then the onset of administration may not be delayed. However, the dosage amount or frequency of dosing of the one or more TGF-beta superfamily heteromultimers of the disclosure may be set at an amount that would reduce the risk of an unacceptable increase in the hematologic parameters arising upon administration of the one or more TGF-beta superfamily heteromultimers of the disclosure. Alternatively, a therapeutic regimen may be developed for the patient that combines one or more TGF-beta superfamily heteromultimers of the disclosure with a therapeutic agent that addresses the undesirable level of the hematologic parameter. For example, if the patient has elevated blood pressure, then a therapeutic regimen involving administration of one or more TGF-beta superfamily heteromultimers of the disclosure and a blood pressure-lowering agent may be designed. For a patient having lower than desired iron stores, a therapeutic regimen of one or more TGF-beta superfamily heteromultimers of the disclosure and iron supplementation may be developed.

In one embodiment, baseline parameter(s) for one or more hematologic parameters may be established for a patient who is a candidate to be treated with one or more TGF-beta superfamily heteromultimers of the disclosure and an appropriate dosing regimen established for that patient based on the baseline value(s). Alternatively, established baseline parameters based on a patient's medical history could be used to inform an appropriate dosing regimen for a patient. For example, if a healthy patient has an established baseline blood pressure reading that is above the defined normal range it may not be necessary to bring the patient's blood pressure into the range that is considered normal for the general population prior to treatment with the one or more TGF-beta superfamily heteromultimers of the disclosure. A patient's baseline values for one or more hematologic parameters prior to treatment with one or more TGF-beta superfamily heteromultimers of the disclosure may also be used as the relevant comparative values for monitoring any changes to the hematologic parameters during treatment with the one or more TGF-beta superfamily heteromultimers of the disclosure.

In certain embodiments, one or more hematologic parameters are measured in patients who are being treated with a one or more TGF-beta superfamily heteromultimers of the disclosure. The hematologic parameters may be used to monitor the patient during treatment and permit adjustment or termination of the dosing with the one or more TGF-beta superfamily heteromultimers of the disclosure or additional dosing with another therapeutic agent. For example, if administration of one or more TGF-beta superfamily heteromultimer complexes of the disclosure of the disclosure results in an increase in blood pressure, red blood cell level, or hemoglobin level, or a reduction in iron stores, then the dose of the one or more TGF-beta superfamily heteromultimers of the disclosure may be reduced in amount or frequency in order to decrease the effects of the one or more TGF-beta superfamily heteromultimers of the disclosure on the one or more hematologic parameters. If administration of one or more TGF-beta superfamily heteromultimers of the disclosure results in a change in one or more hematologic parameters that is adverse to the patient, then the dosing of the one or more TGF-beta superfamily heteromultimers of the disclosure may be terminated either temporarily, until the hematologic parameter(s) return to an acceptable level, or permanently. Similarly, if one or more hematologic parameters are not brought within an acceptable range after reducing the dose or frequency of administration of the one or more TGF-beta superfamily heteromultimers of the disclosure, then the dosing may be terminated. As an alternative, or in addition to, reducing or terminating the dosing with the one or more TGF-beta superfamily heteromultimers of the disclosure, the patient may be dosed with an additional therapeutic agent that addresses the undesirable level in the hematologic parameter(s), such as, for example, a blood pressure-lowering agent or an iron supplement. For example, if a patient being treated with one or more TGF-beta superfamily heteromultimers of the disclosure has elevated blood pressure, then dosing with the one or more TGF-beta superfamily heteromultimers of the disclosure may continue at the same level and a blood pressure-lowering agent is added to the treatment regimen, dosing with the one or more TGF-beta superfamily heteromultimers of the disclosure may be reduced (e.g., in amount and/or frequency) and a blood pressure-lowering agent is added to the treatment regimen, or dosing with the one or more TGF-beta superfamily heteromultimers of the disclosure may be terminated and the patient may be treated with a blood pressure-lowering agent.

### 6. Pharmaceutical Compositions

In certain aspects, TGF-beta superfamily heteromultimers (e.g., TGF-beta superfamily co-receptor heteromultimers) of the present disclosure can be administered alone or as a component of a pharmaceutical formulation (also referred to as a therapeutic composition or pharmaceutical composition). A pharmaceutical formation refers to a preparation which is in such form as to permit the biological activity of an active ingredient (e.g., an agent of the present disclosure) contained therein to be effective and which contains no additional components which are unacceptably toxic to a subject to which the formulation would be administered. The subject compounds may be formulated for administration in any convenient way for use in human or veterinary medicine. For example, one or more agents of the present disclosure may be formulated with a pharmaceutically acceptable carrier. A pharmaceutically acceptable carrier refers to an ingredient in a pharmaceutical formulation, other than an active ingredient, which is generally nontoxic to a subject. A pharmaceutically acceptable carrier includes, but is not limited to, a buffer, excipient, stabilizer, and/or preservative. In some embodiments, pharmaceutical formulations for use in the present disclosure are in a pyrogen-free, physiologically-acceptable form when administered to a subject. Therapeutically useful agents other than those described herein, which may optionally be included in the formulation as described above, may be administered in combination with the subject agents in the methods of the present disclosure.

In certain embodiments, compositions will be administered parenterally [e.g., by intravenous (I.V.) injection, intraarterial injection, intraosseous injection, intramuscular injection, intrathecal injection, subcutaneous injection, or intradermal injection]. Pharmaceutical compositions suitable for parenteral administration may comprise one or more agents of the disclosure in combination with one or more pharmaceutically acceptable sterile isotonic aqueous or nonaqueous solutions, dispersions, suspensions or emulsions, or sterile powders which may be reconstituted into sterile injectable solutions or dispersions just prior to use. Injectable solutions or dispersions may contain antioxidants, buffers, bacteriostats, suspending agents, thickening agents, or solutes which render the formulation isotonic with the blood of the intended recipient. Examples of suitable aqueous and nonaqueous carriers which may be employed in the pharmaceutical formulations of the present disclosure include water, ethanol, polyols (e.g., glycerol, propylene glycol, polyethylene glycol, etc.), vegetable oils (e.g., olive oil), injectable organic esters (e.g., ethyl oleate), and suitable mixtures thereof. Proper fluidity can be maintained, for example, by the use of coating materials (e.g., lecithin), by the maintenance of the required particle size in the case of dispersions, and by the use of surfactants.

In some embodiments, a therapeutic method of the present disclosure includes administering the pharmaceutical composition systemically, or locally, from an implant or device. Further, the pharmaceutical composition may be encapsulated or injected in a form for delivery to a target tissue site (e.g., bone marrow or muscle). In certain embodiments, compositions of the present disclosure may include a matrix capable of delivering one or more of the agents of the present disclosure to a target tissue site (e.g., bone marrow or muscle), providing a structure for the developing tissue and optimally capable of being resorbed into the body. For example, the matrix may provide slow release of one or more agents of the present disclosure. Such matrices may be formed of materials presently in use for other implanted medical applications.

The choice of matrix material may be based on one or more of: biocompatibility, biodegradability, mechanical properties, cosmetic appearance, and interface properties. The particular application of the subject compositions will define the appropriate formulation. Potential matrices for the compositions may be biodegradable and chemically defined calcium sulfate, tricalciumphosphate, hydroxyapatite, polylactic acid, and polyanhydrides. Other potential materials are biodegradable and biologically well-defined including, for example, bone or dermal collagen. Further matrices are comprised of pure proteins or extracellular matrix components. Other potential matrices are non-biodegradable and chemically defined including, for example, sintered hydroxyapatite, bioglass, aluminates, or other ceramics. Matrices may be comprised of combinations of any of the above mentioned types of material including, for example, polylactic acid and hydroxyapatite or collagen and tricalciumphosphate. The bioceramics may be altered in composition (e.g., calcium-aluminate-phosphate) and processing to alter one or more of pore size, particle size, particle shape, and biodegradability.

In certain embodiments, pharmaceutical compositions of present disclosure can be administered topically. “Topical application” or “topically” means contact of the pharmaceutical composition with body surfaces including, for example, the skin, wound sites, and mucous membranes. The topical pharmaceutical compositions can have various application forms and typically comprises a drug-containing layer, which is adapted to be placed near to or in direct contact with the tissue upon topically administering the composition. Pharmaceutical compositions suitable for topical administration may comprise one or more one or more TGFβ superfamily heteromultimers of the disclosure in combination formulated as a liquid, a gel, a cream, a lotion, an ointment, a foam, a paste, a putty, a semi-solid, or a solid. Compositions in the liquid, gel, cream, lotion, ointment, foam, paste, or putty form can be applied by spreading, spraying, smearing, dabbing or rolling the composition on the target tissue. The compositions also may be impregnated into sterile dressings, transdermal patches, plasters, and bandages. Compositions of the putty, semi-solid or solid forms may be deformable. They may be elastic or non-elastic (e.g., flexible or rigid). In certain aspects, the composition forms part of a composite and can include fibers, particulates, or multiple layers with the same or different compositions.

Topical compositions in the liquid form may include pharmaceutically acceptable solutions, emulsions, microemulsions, and suspensions. In addition to the active ingredient(s), the liquid dosage form may contain an inert diluent commonly used in the art including, for example, water or other solvent, a solubilizing agent and/or emulsifier [e.g., ethyl alcohol, isopropyl alcohol, ethyl carbonate, ethyl acetate, benzyl alcohol, benzyl benzoate, propylene glycol, or 1,3-butylene glycol, an oil (e.g., cottonseed, groundnut, corn, germ, olive, castor, and sesame oil), glycerol, tetrahydrofuryl alcohol, a polyethylene glycol, a fatty acid ester of sorbitan, and mixtures thereof].

Topical gel, cream, lotion, ointment, semi-solid or solid compositions may include one or more thickening agents, such as a polysaccharide, synthetic polymer or protein-based polymer. In one embodiment of the invention, the gelling agent herein is one that is suitably nontoxic and gives the desired viscosity. The thickening agents may include polymers, copolymers, and monomers of: vinylpyrrolidones, methacrylamides, acrylamides N-vinylimidazoles, carboxy vinyls, vinyl esters, vinyl ethers, silicones, polyethyleneoxides, polyethyleneglycols, vinylalcohols, sodium acrylates, acrylates, maleic acids, NN-dimethylacrylamides, diacetone acrylamides, acrylamides, acryloyl morpholine, pluronic, collagens, polyacrylamides, polyacrylates, polyvinyl alcohols, polyvinylenes, polyvinyl silicates, polyacrylates substituted with a sugar (e.g., sucrose, glucose, glucosamines, galactose, trehalose, mannose, or lactose), acylamidopropane sulfonic acids, tetramethoxyorthosilicates, methyltrimethoxyorthosilicates, tetraalkoxyorthosilicates, trialkoxyorthosilicates, glycols, propylene glycol, glycerine, polysaccharides, alginates, dextrans, cyclodextrin, celluloses, modified celluloses, oxidized celluloses, chitosans, chitins, guars, carrageenans, hyaluronic acids, inulin, starches, modified starches, agarose, methylcelluloses, plant gums, hylaronans, hydrogels, gelatins, glycosaminoglycans, carboxymethyl celluloses, hydroxyethyl celluloses, hydroxy propyl methyl celluloses, pectins, low-methoxy pectins, cross-linked dextrans, starch-acrylonitrile graft copolymers, starch sodium polyacrylate, hydroxyethyl methacrylates, hydroxyl ethyl acrylates, polyvinylene, polyethylvinylethers, polymethyl methacrylates, polystyrenes, polyurethanes, polyalkanoates, polylactic acids, polylactates, poly(3-hydroxybutyrate), sulfonated hydrogels, AMPS (2-acrylamido-2-methyl-1-propanesulfonic acid), SEM (sulfoethylmethacrylate), SPM (sulfopropyl methacrylate), SPA (sulfopropyl acrylate), N,N-dimethyl-N-methacryloxyethyl-N-(3-sulfopropyl)ammonium betaine, methacryllic acid amidopropyl-dimethyl ammonium sulfobetaine, SPI (itaconic acid-bis(1-propyl sulfonizacid-3) ester di-potassium salt), itaconic acids, AMBC (3-acrylamido-3-methylbutanoic acid), beta-carboxyethyl acrylate (acrylic acid dimers), and maleic anhydride-methylvinyl ether polymers, derivatives thereof, salts thereof, acids thereof, and combinations thereof. In certain embodiments, pharmaceutical compositions of present disclosure can be administered orally, for example, in the form of capsules, cachets, pills, tablets, lozenges (using a flavored basis such as sucrose and acacia or tragacanth), powders, granules, a solution or a suspension in an aqueous or non-aqueous liquid, an oil-in-water or water-in-oil liquid emulsion, or an elixir or syrup, or pastille (using an inert base, such as gelatin and glycerin, or sucrose and acacia), and/or a mouth wash, each containing a predetermined amount of a compound of the present disclosure and optionally one or more other active ingredients. A compound of the present disclosure and optionally one or more other active ingredients may also be administered as a bolus, electuary, or paste.

In solid dosage forms for oral administration (e.g., capsules, tablets, pills, dragees, powders, and granules), one or more compounds of the present disclosure may be mixed with one or more pharmaceutically acceptable carriers including, for example, sodium citrate, dicalcium phosphate, a filler or extender (e.g., a starch, lactose, sucrose, glucose, mannitol, and silicic acid), a binder (e.g. carboxymethylcellulose, an alginate, gelatin, polyvinyl pyrrolidone, sucrose, and acacia), a humectant (e.g., glycerol), a disintegrating agent (e.g., agar-agar, calcium carbonate, potato or tapioca starch, alginic acid, a silicate, and sodium carbonate), a solution retarding agent (e.g. paraffin), an absorption accelerator (e.g. a quaternary ammonium compound), a wetting agent (e.g., cetyl alcohol and glycerol monostearate), an absorbent (e.g., kaolin and bentonite clay), a lubricant (e.g., a talc, calcium stearate, magnesium stearate, solid polyethylene glycols, sodium lauryl sulfate), a coloring agent, and mixtures thereof. In the case of capsules, tablets, and pills, the pharmaceutical formulation (composition) may also comprise a buffering agent. Solid compositions of a similar type may also be employed as fillers in soft and hard-filled gelatin capsules using one or more excipients including, e.g., lactose or a milk sugar as well as a high molecular-weight polyethylene glycol.

Liquid dosage forms for oral administration of the pharmaceutical composition may include pharmaceutically acceptable emulsions, microemulsions, solutions, suspensions, syrups, and elixirs. In addition to the active ingredient(s), the liquid dosage form may contain an inert diluent commonly used in the art including, for example, water or other solvent, a solubilizing agent and/or emulsifier [e.g., ethyl alcohol, isopropyl alcohol, ethyl carbonate, ethyl acetate, benzyl alcohol, benzyl benzoate, propylene glycol, or 1,3-butylene glycol, an oil (e.g., cottonseed, groundnut, corn, germ, olive, castor, and sesame oil), glycerol, tetrahydrofuryl alcohol, a polyethylene glycol, a fatty acid ester of sorbitan, and mixtures thereof]. Besides inert diluents, the oral formulation can also include an adjuvant including, for example, a wetting agent, an emulsifying and suspending agent, a sweetening agent, a flavoring agent, a coloring agent, a perfuming agent, a preservative agent, and combinations thereof.

Suspensions, in addition to the active compounds, may contain suspending agents including, for example, an ethoxylated isostearyl alcohol, polyoxyethylene sorbitol, a sorbitan ester, microcrystalline cellulose, aluminum metahydroxide, bentonite, agar-agar, tragacanth, and combinations thereof.

Prevention of the action and/or growth of microorganisms may be ensured by the inclusion of various antibacterial and antifungal agents including, for example, paraben, chlorobutanol, and phenol sorbic acid.

In certain embodiments, it may be desirable to include an isotonic agent including, for example, a sugar or sodium chloride into the compositions. In addition, prolonged absorption of an injectable pharmaceutical form may be brought about by the inclusion of an agent that delay absorption including, for example, aluminum monostearate and gelatin.

It is understood that the dosage regimen will be determined by the attending physician considering various factors which modify the action of the one or more of the agents of the present disclosure. In the case of a TGF-beta superfamily heteromultimer that promotes red blood cell formation, various factors may include, but are not limited to, the patient's red blood cell count, hemoglobin level, the desired target red blood cell count, the patient's age, the patient's sex, the patient's diet, the severity of any disease that may be contributing to a depressed red blood cell level, the time of administration, and other clinical factors. The addition of other known active agents to the final composition may also affect the dosage. Progress can be monitored by periodic assessment of one or more of red blood cell levels, hemoglobin levels, reticulocyte levels, and other indicators of the hematopoietic process.

In certain embodiments, the present disclosure also provides gene therapy for the in vivo production of one or more of the agents of the present disclosure. Such therapy would achieve its therapeutic effect by introduction of the agent sequences into cells or tissues having one or more of the disorders as listed above. Delivery of the agent sequences can be achieved, for example, by using a recombinant expression vector such as a chimeric virus or a colloidal dispersion system. Preferred therapeutic delivery of one or more of agent sequences of the disclosure is the use of targeted liposomes.

Various viral vectors which can be utilized for gene therapy as taught herein include adenovirus, herpes virus, vaccinia, or an RNA virus (e.g., a retrovirus). The retroviral vector may be a derivative of a murine or avian retrovirus. Examples of retroviral vectors in which a single foreign gene can be inserted include, but are not limited to: Moloney murine leukemia virus (MoMuLV), Harvey murine sarcoma virus (HaMuSV), murine mammary tumor virus (MuMTV), and Rous Sarcoma Virus (RSV). A number of additional retroviral vectors can incorporate multiple genes. All of these vectors can transfer or incorporate a gene for a selectable marker so that transduced cells can be identified and generated. Retroviral vectors can be made target-specific by attaching, for example, a sugar, a glycolipid, or a protein. Preferred targeting is accomplished by using an antibody. Those of skill in the art will recognize that specific polynucleotide sequences can be inserted into the retroviral genome or attached to a viral envelope to allow target specific delivery of the retroviral vector containing one or more of the agents of the present disclosure.

Alternatively, tissue culture cells can be directly transfected with plasmids encoding the retroviral structural genes (gag, pol, and env), by conventional calcium phosphate transfection. These cells are then transfected with the vector plasmid containing the genes of interest. The resulting cells release the retroviral vector into the culture medium.

Another targeted delivery system for one or more of the agents of the present disclosure is a colloidal dispersion system. Colloidal dispersion systems include, for example, macromolecule complexes, nanocapsules, microspheres, beads, and lipid-based systems including oil-in-water emulsions, micelles, mixed micelles, and liposomes. In certain embodiments, the preferred colloidal system of this disclosure is a liposome. Liposomes are artificial membrane vesicles which are useful as delivery vehicles in vitro and in vivo. RNA, DNA, and intact virions can be encapsulated within the aqueous interior and be delivered to cells in a biologically active form. See, e.g., Fraley, et al. (1981) Trends Biochem. Sci., 6:77. Methods for efficient gene transfer using a liposome vehicle are known in the art. See, e.g., Mannino, et al. (1988) Biotechniques, 6:682, 1988.

The composition of the liposome is usually a combination of phospholipids, which may include a steroid (e.g. cholesterol). The physical characteristics of liposomes depend on pH, ionic strength, and the presence of divalent cations. Other phospholipids or other lipids may also be used including, for example a phosphatidyl compound (e.g., phosphatidylglycerol, phosphatidylcholine, phosphatidylserine, phosphatidylethanolamine, a sphingolipid, a cerebroside, and a ganglioside), egg phosphatidylcholine, dipalmitoylphosphatidylcholine, and distearoylphosphatidylcholine. The targeting of liposomes is also possible based on, for example, organ-specificity, cell-specificity, and organelle-specificity and is known in the art.

### EXEMPLIFICATION

The invention now being generally described, it will be more readily understood by reference to the following examples, which are included merely for purposes of illustration of certain embodiments and embodiments of the present invention, and are not intended to limit the invention.

### Example 1. Generation of an ENG-Fc:ALK1-Fc Heterodimer

A soluble ENG-Fc:ALK1-Fc heteromultimer can be generated comprising a C-terminally truncated extracellular domain of human endoglin (ENG) and the extracellular domain of human ALK1, which are each fused to an Fc domain with a linker positioned between the extracellular domain and the Fc domain. The individual constructs are referred to as ENG(26-346)-Fc and ALK1-Fc fusion proteins, respectively. Other ENG-Fc constructs known in the art could similarly be used to generate ENG-Fc:ALK1-Fc heteromultimers. See, e.g., U.S. Ser. Nos. 14/112,620 and 14/522,891, the contents thereof are incorporated herein in their entirety.

A methodology for promoting formation of ENG-Fc:ALK1-Fc heteromultimers, as opposed to ENG-Fc or ALK1-Fc homomultimers, is to introduce alterations in the amino acid sequence of the Fc domains to guide the formation of asymmetric heteromultimers. Many different approaches to making asymmetric interaction pairs using Fc domains are described in this disclosure.

In one approach, illustrated in the ENG-Fc and ALK1-Fc polypeptide sequences of SEQ ID NOs: 101-103 and 104-106, respectively, one Fc domain is altered to introduce cationic amino acids at the interaction face, while the other Fc domain is altered to introduce anionic amino acids at the interaction face. The ENG(26-346)-Fc fusion polypeptide and ALK1-Fc fusion polypeptide each employ the tissue plasminogen activator (TPA) leader:

(SEQ ID NO: 100)

MDAMKRGLCCVLLLCGAVFVSP.

The ENG(26-346)-Fc polypeptide sequence (SEQ ID NO: 101) is shown below:

| (SEQ ID NO: 101) | |
|---|---|
| 1 | MDAMKRGLCC VLLLCGAVFV SPGAETVHCD LQPVGPERDE VTYTTSQVSK |
| 51 | GCVAQAPNAI LEVHVLFLEF PTGPSQLELT LQASKQNGTW PREVLLVLSV |
| 101 | NSSVFLHLQA LGIPLHLAYN SSLVTFQEPP GVNTTELPSF PKTQILEWAA |
| 151 | ERGPITSAAE LNDPQSILLR LGQAQGSLSF CMLEASQDMG RTLEWRPRTP |
| 201 | ALVRGCHLEG VAGHKEAHIL RVLPGHSAGP RTVTVKVELS CAPGDLDAVL |
| 251 | ILQGPPYVSW LIDANHNMQI WTTGEYSFKI FPEKNIRGFK LPDTPQGLLG |
| 301 | EARMLNASIV ASFVELPLAS IVSLHASSCG GRLQTSPAPI QTTPPTGGGT |
| 351 | HTCPPCPAPE LLGGPSVFLF PPKPKDTLMI SRTPEVTCVV VDVSHEDPEV |
| 401 | KFNWYVDGVE VHNAKTKPRE EQYNSTYRVV SVLTVLHQDW LNGKEYKCKV |
| 451 | SNKALPAPIE KTISKAKGQP REPQVYTLPP SRKEMTKNQV SLTCLVKGFY |
| 501 | PSDIAVEWES NGQPENNYKT TPPVLKSDGS FFLYSKLTVD KSRWQQGNVF |
| 551 | SCSVMHEALH NHYTQKSLSL SPGK |

The leader sequence and linker sequence are underlined. To promote formation of the ENG(26-346)-Fc:ALK1-Fc heterodimer rather than either of the possible homodimeric complexes, two amino acid substitutions (replacing acidic amino acids with lysine) can be introduced into the Fc domain of the ENG(26-346)-Fc fusion protein as indicated by double underline above. The amino acid sequence of SEQ ID NO: 101 may optionally be provided with the lysine removed from the C-terminus.

This ENG(26-346)-Fc fusion protein is encoded by the following nucleic acid sequence (SEQ ID NO: 102):

| (SEQ ID NO: 102) | |
|---|---|
| 1 | ATGGATGCAA TGAAGAGAGG GCTCTGCTGT GTGCTGCTGC TGTGTGGAGC |
| 51 | AGTCTTCGTT TCGCCCGGCG CCGAAACAGT CCATTGTGAC CTTCAGCCTG |
| 101 | TGGGCCCCGA GAGGGACGAG GTGACATATA CCACTAGCCA GGTCTCGAAG |
| 151 | GGCTGCGTGG CTCAGGCCCC CAATGCCATC CTTGAAGTCC ATGTCCTCTT |
| 201 | CCTGGAGTTC CCAACGGGCC CGTCACAGCT GGAGCTGACT CTCCAGGCAT |
| 251 | CCAAGCAAAA TGGCACCTGG CCCCGAGAGG TGCTTCTGGT CCTCAGTGTA |
| 301 | AACAGCAGTG TCTTCCTGCA TCTCCAGGCC CTGGGAATCC CACTGCACTT |
| 351 | GGCCTACAAT TCCAGCCTGG TCACCTTCCA AGAGCCCCCG GGGGTCAACA |
| 401 | CCACAGAGCT GCCATCCTTC CCCAAGACCC AGATCCTTGA GTGGGCAGCT |
| 451 | GAGAGGGGCC CCATCACCTC TGCTGCTGAG CTGAATGACC CCCAGAGCAT |
| 501 | CCTCCTCCGA CTGGGCCAAG CCCAGGGGTC ACTGTCCTTC TGCATGCTGG |
| 551 | AAGCCAGCCA GGACATGGGC CGCACGCTCG AGTGGCGGCC GCGTACTCCA |
| 601 | GCCTTGGTCC GGGGCTGCCA CTTGGAAGGC GTGGCCGGCC ACAAGGAGGC |
| 651 | GCACATCCTG AGGGTCCTGC CGGGCCACTC GGCCGGGCCC CGGACGGTGA |
| 701 | CGGTGAAGGT GGAACTGAGC TGCGCACCCG GGGATCTCGA TGCCGTCCTC |
| 751 | ATCCTGCAGG GTCCCCCCTA CGTGTCCTGG CTCATCGACG CCAACCACAA |
| 801 | CATGCAGATC TGGACCACTG GAGAATACTC CTTCAAGATC TTTCCAGAGA |
| 851 | AAAACATTCG TGGCTTCAAG CTCCCAGACA CACCTCAAGG CCTCCTGGGG |
| 901 | GAGGCCCGGA TGCTCAATGC CAGCATTGTG GCATCCTTCG TGGAGCTACC |
| 951 | GCTGGCCAGC ATTGTCTCAC TTCATGCCTC CAGCTGCGGT GGTAGGCTGC |
| 1001 | AGACCTCACC CGCACCGATC CAGACCACTC CTCCCACCGG TGGTGGAACT |
| 1051 | CACACATGCC CACCGTGCCC AGCACCTGAA CTCCTGGGGG GACCGTCAGT |
| 1101 | CTTCCTCTTC CCCCCAAAAC CCAAGGACAC CCTCATGATC TCCCGGACCC |
| 1151 | CTGAGGTCAC ATGCGTGGTG GTGGACGTGA GCCACGAAGA CCCTGAGGTC |
| 1201 | AAGTTCAACT GGTACGTGGA CGGCGTGGAG GTGCATAATG CCAAGACAAA |
| 1251 | GCCGCGGGAG GAGCAGTACA ACAGCACGTA CCGTGTGGTC AGCGTCCTCA |
| 1301 | CCGTCCTGCA CCAGGACTGG CTGAATGGCA AGGAGTACAA GTGCAAGGTC |
| 1351 | TCCAACAAAG CCCTCCCAGC CCCCATCGAG AAAACCATCT CCAAAGCCAA |
| 1401 | AGGGCAGCCC CGAGAACCAC AGGTGTACAC CCTGCCCCCA TCCCGGAAGG |
| 1451 | AGATGACCAA GAACCAGGTC AGCCTGACCT GCCTGGTCAA AGGCTTCTAT |
| 1501 | CCCAGCGACA TCGCCGTGGA GTGGGAGAGC AATGGGCAGC CGGAGAACAA |
| 1551 | CTACAAGACC ACGCCTCCCG TGCTGAAGTC CGACGGCTCC TTCTTCCTCT |
| 1601 | ATAGCAAGCT CACCGTGGAC AAGAGCAGGT GGCAGCAGGG GAACGTCTTC |
| 1651 | TCATGCTCCG TGATGCATGA GGCTCTGCAC AACCACTACA CGCAGAAGAG |
| 1701 | CCTCTCCCTG TCCCCGGGTA AA |

The mature ENG(26-346)-Fc fusion polypeptide (SEQ ID NO: 103) is as follows and may optionally be provided with the lysine removed from the C-terminus.

| (SEQ ID NO: 103) | |
|---|---|
| 1 | ETVHCDLQPV GPERDEVTYT TSQVSKGCVA QAPNAILEVH VLFLEFPTGP |
| 51 | SQLELTLQAS KQNGTWPREV LLVLSVNSSV FLHLQALGIP LHLAYNSSLV |
| 101 | TFQEPPGVNT TELPSFPKTQ ILEWAAERGP ITSAAELNDP QSILLRLGQA |
| 151 | QGSLSFCMLE ASQDMGRTLE WRPRTPALVR GCHLEGVAGH KEAHILRVLP |
| 201 | GHSAGPRTVT VKVELSCAPG DLDAVLILQG PPYVSWLIDA NHNMQIWTTG |
| 251 | EYSFKIFPEK NIRGFKLPDT PQGLLGEARM LNASIVASFV ELPLASIVSL |
| 301 | HASSCGGRLQ TSPAPIQTTP PTGGGTHTCP PCPAPELLGG PSVFLFPPKP |
| 351 | KDTLMISRTP EVTCVVVDVS HEDPEVKFNW YVDGVEVHNA KTKPREEQYN |
| 401 | STYRVVSVLT VLHQDWLNGK EYKCKVSNKA LPAPIEKTIS KAKGQPREPQ |
| 451 | VYTLPPSRKE MTKNQVSLTC LVKGFYPSDI AVEWESNGQP ENNYKTTPPV |
| 501 | LKSDGSFFLY SKLTVDKSRW QQGNVFSCSV MHEALHNHYT QKSLSLSPGK |

The complementary form of ALK1-Fc fusion polypeptide (SEQ ID NO: 104) is as follows:

| (SEQ ID NO: 104) | |
|---|---|
| 1 | MDAMKRGLCC VLLLCGAVFV SPGADPVKPS RGPLVTCTCE SPHCKGPTCR |
| 51 | GAWCTVVLVR EEGRHPQEHR GCGNLHRELC RGRPTEFVNH YCCDSHLCNH |
| 101 | NVSLVLEATQ PPSEQPGTDG QLATGGGTHT CPPCPAPELL GGPSVFLFPP |
| 151 | KPKDTLMISR TPEVTCVVVD VSHEDPEVKF NWYVDGVEVH NAKTKPREEQ |
| 201 | YNSTYRVVSV LTVLHQDWLN GKEYKCKVSN KALPAPIEKT ISKAKGQPRE |
| 251 | PQVYTLPPSR EEMTKNQVSL TCLVKGFYPS DIAVEWESNG QPENNYDTTP |
| 301 | PVLDSDGSFF LYSDLTVDKS RWQQGNVFSC SVMHEALHNH YTQKSLSLSP |
| 351 | G |

The leader sequence and linker sequence are underlined. To guide heterodimer formation with the ENG(26-346)-Fc fusion polypeptide of SEQ ID NOs: 101 and 103 above, two amino acid substitutions (replacing lysines with aspartic acids) can be introduced into the Fc domain of the ALK1-Fc fusion polypeptide as indicated by double underline above. The amino acid sequence of SEQ ID NO: 104 may optionally be provided with a lysine added at the C-terminus.

This ALK1-Fc fusion protein is encoded by the following nucleic acid (SEQ ID NO: 105):

| (SEQ ID NO: 105) | |
|---|---|
| 1 | ATGGATGCAA TGAAGAGAGG GCTCTGCTGT GTGCTGCTGC TGTGTGGAGC |
| 51 | AGTCTTCGTT TCGCCCGGCG CCGACCCTGT GAAGCCGTCT CGGGGCCCGC |
| 101 | TGGTGACCTG CACGTGTGAG AGCCCACATT GCAAGGGGCC TACCTGCCGG |
| 151 | GGGGCCTGGT GCACAGTAGT GCTGGTGCGG GAGGAGGGGA GGCACCCCCA |
| 201 | GGAACATCGG GGCTGCGGGA ACTTGCACAG GGAGCTCTGC AGGGGCCGCC |
| 251 | CCACCGAGTT CGTCAACCAC TACTGCTGCG ACAGCCACCT CTGCAACCAC |
| 301 | AACGTGTCCC TGGTGCTGGA GGCCACCCAA CCTCCTTCGG AGCAGCCGGG |
| 351 | AACAGATGGC CAGCTGGCCA CCGGTGGTGG AACTCACACA TGCCCACCGT |
| 401 | GCCCAGCACC TGAACTCCTG GGGGGACCGT CAGTCTTCCT CTTCCCCCCA |
| 451 | AAACCCAAGG ACACCCTCAT GATCTCCCGG ACCCCTGAGG TCACATGCGT |
| 501 | GGTGGTGGAC GTGAGCCACG AAGACCCTGA GGTCAAGTTC AACTGGTACG |
| 551 | TGGACGGCGT GGAGGTGCAT AATGCCAAGA CAAAGCCGCG GGAGGAGCAG |
| 601 | TACAACAGCA CGTACCGTGT GGTCAGCGTC CTCACCGTCC TGCACCAGGA |
| 651 | CTGGCTGAAT GGCAAGGAGT ACAAGTGCAA GGTCTCCAAC AAAGCCCTCC |
| 701 | CAGCCCCCAT CGAGAAAACC ATCTCCAAAG CCAAAGGGCA GCCCCGAGAA |
| 751 | CCACAGGTGT ACACCCTGCC CCCATCCCGG GAGGAGATGA CCAAGAACCA |
| 801 | GGTCAGCCTG ACCTGCCTGG TCAAAGGCTT CTATCCCAGC GACATCGCCG |
| 851 | TGGAGTGGGA GAGCAATGGG CAGCCGGAGA ACAACTACGA CACCACGCCT |
| 901 | CCCGTGCTGG ACTCCGACGG CTCCTTCTTC CTCTATAGCG ACCTCACCGT |
| 951 | GGACAAGAGC AGGTGGCAGC AGGGGAACGT CTTCTCATGC TCCGTGATGC |
| 1001 | ATGAGGCTCT GCACAACCAC TACACGCAGA AGAGCCTCTC CCTGTCTCCG |
| 1051 | GGT |

A processed ALK1-Fc fusion protein sequence (SEQ ID NO: 106) is as follows and may optionally be provided with a lysine added at the C-terminus.

| (SEQ ID NO: 106) | |
|---|---|
| 1 | DPVKPSRGPL VTCTCESPHC KGPTCRGAWC TVVLVREEGR HPQEHRGCGN |
| 51 | LHRELCRGRP TEFVNHYCCD SHLCNHNVSL VLEATQPPSE QPGTDGQLAT |
| 101 | GGGTHTCPPC PAPELLGGPS VFLFPPKPKD TLMISRTPEV TCVVVDVSHE |
| 151 | DPEVKFNWYV DGVEVHNAKT KPREEQYNST YRVVSVLTVL HQDWLNGKEY |
| 201 | KCKVSNKALP APIEKTISKA KGQPREPQVY TLPPSREEMT KNQVSLTCLV |
| 251 | KGFYPSDIAV EWESNGQPEN NYDTTPPVLD SDGSFFLYSD LTVDKSRWQQ |
| 301 | GNVFSCSVMH EALHNHYTQK SLSLSPG |

The ENG(26-346)-Fc and ALK1-Fc proteins of SEQ ID NO: 103 and SEQ ID NO: 106, respectively, may be co-expressed and purified from a CHO cell line, to give rise to a heteromultimer comprising ENG(26-346)-Fc:ALK1-Fc.

In a second approach to promote the formation of heteromultimer using asymmetric Fc fusion proteins, the Fc domains are altered to introduce complementary hydrophobic interactions and an additional intermolecular disulfide bond.

The ENG(26-346)-Fc polypeptide sequence (SEQ ID NO: 801) is shown below:

| (SEQ ID NO: 801) | |
|---|---|
| 1 | MDAMKRGLCC VLLLCGAVFV SPGAETVHCD LQPVGPERDE VTYTTSQVSK |
| 51 | GCVAQAPNAI LEVHVLFLEF PTGPSQLELT LQASKQNGTW PREVLLVLSV |
| 101 | NSSVFLHLQA LGIPLHLAYN SSLVTFQEPP GVNTTELPSF PKTQILEWAA |
| 151 | ERGPITSAAE LNDPQSILLR LGQAQGSLSF CMLEASQDMG RTLEWRPRTP |
| 201 | ALVRGCHLEG VAGHKEAHIL RVLPGHSAGP RTVTVKVELS CAPGDLDAVL |
| 251 | ILQGPPYVSW LIDANHNMQI WTTGEYSFKI FPEKNIRGFK LPDTPQGLLG |
| 301 | EARMLNASIV ASFVELPLAS IVSLHASSCG GRLQTSPAPI QTTPPTGGGT |
| 351 | HTCPPCPAPE LLGGPSVFLF PPKPKDTLMI SRTPEVTCVV VDVSHEDPEV |
| 401 | KFNWYVDGVE VHNAKTKPRE EQYNSTYRVV SVLTVLHQDW LNGKEYKCKV |
| 451 | SNKALPAPIE KTISKAKGQP REPQVYTLPP CREEMTKNQV SLWCLVKGFY |
| 501 | PSDIAVEWES NGQPENNYKT TPPVLDSDGS FFLYSKLTVD KSRWQQGNVF |
| 551 | SCSVMHEALH NHYTQKSLSL SPGK |

The leader sequence and linker sequence are underlined. To promote formation of the ENG(26-346)-Fc:ALK1-Fc heterodimer rather than either of the possible homodimeric complexes, two amino acid substitutions (replacing a serine with a cysteine and a threonine with a tryptophan) can be introduced into the Fc domain of the fusion protein as indicated by double underline above. The amino acid sequence of SEQ ID NO: 801 may optionally be provided with the lysine removed from the C-terminus.

A processed ENG(26-346)-Fc fusion polypeptide (SEQ ID NO: 802) is as follows and may optionally be provided with the lysine (K) removed from the C-terminus.

| (SEQ ID NO: 802) | |
|---|---|
| 1 | ETVHCDLQPV GPERDEVTYT TSQVSKGCVA QAPNAILEVH VLFLEFPTGP |
| 51 | SQLELTLQAS KQNGTWPREV LLVLSVNSSV FLHLQALGIP LHLAYNSSLV |
| 101 | TFQEPPGVNT TELPSFPKTQ ILEWAAERGP ITSAAELNDP QSILLRLGQA |
| 151 | QGSLSFCMLE ASQDMGRTLE WRPRTPALVR GCHLEGVAGH KEAHILRVLP |
| 201 | GHSAGPRTVT VKVELSCAPG DLDAVLILQG PPYVSWLIDA NHNMQIWTTG |
| 251 | EYSFKIFPEK NIRGFKLPDT PQGLLGEARM LNASIVASFV ELPLASIVSL |
| 301 | HASSCGGRLQ TSPAPIQTTP PTGGGTHTCP PCPAPELLGG PSVFLFPPKP |
| 351 | KDTLMISRTP EVTCVVVDVS HEDPEVKFNW YVDGVEVHNA KTKPREEQYN |
| 401 | STYRVVSVLT VLHQDWLNGK EYKCKVSNKA LPAPIEKTIS KAKGQPREPQ |
| 451 | VYTLPPCREE MTKNQVSLWC LVKGFYPSDI AVEWESNGQP ENNYKTTPPV |
| 501 | LDSDGSFFLY SKLTVDKSRW QQGNVFSCSV MHEALHNHYT QKSLSLSPGK |

The complementary form of ALK1-Fc fusion polypeptide (SEQ ID NO: 803) is as follows:

| (SEQ ID NO: 803) | |
|---|---|
| 1 | MDAMKRGLCC VLLLCGAVFV SPGADPVKPS RGPLVTCTCE SPHCKGPTCR |
| 51 | GAWCTVVLVR EEGRHPQEHR GCGNLHRELC RGRPTEFVNH YCCDSHLCNH |
| 101 | NVSLVLEATQ PPSEQPGTDG QLATGGGTHT CPPCPAPELL GGPSVFLFPP |
| 151 | KPKDTLMISR TPEVTCVVVD VSHEDPEVKF NWYVDGVEVH NAKTKPREEQ |
| 201 | YNSTYRVVSV LTVLHQDWLN GKEYKCKVSN KALPAPIEKT ISKAKGQPRE |
| 251 | PQVCTLPPSR EEMTKNQVSL SCAVKGFYPS DIAVEWESNG QPENNYKTTP |
| 301 | PVLDSDGSFF LVSKLTVDKS RWQQGNVFSC SVMHEALHNH YTQKSLSLSP |
| 351 | GK |

The leader sequence and linker sequence are underlined. To guide heterodimer formation with the ENG(26-346)-Fc fusion polypeptide of SEQ ID NOs: 801 and 802 above, four amino acid substitutions can be introduced into the Fc domain of the ALK1 fusion polypeptide as indicated by double underline above. The amino acid sequence of SEQ ID NO: 803 may optionally be provided with the lysine removed from the C-terminus.

A processed ALK1-Fc fusion protein sequence (SEQ ID NO: 804) is as follows and may optionally be provided with the lysine removed from the C-terminus.

| (SEQ ID NO: 804) | |
|---|---|
| 1 | DPVKPSRGPL VTCTCESPHC KGPTCRGAWC TVVLVREEGR HPQEHRGCGN |
| 51 | LHRELCRGRP TEFVNHYCCD SHLCNHNVSL VLEATQPPSE QPGTDGQLAT |
| 101 | GGGTHTCPPC PAPELLGGPS VFLFPPKPKD TLMISRTPEV TCVVVDVSHE |
| 151 | DPEVKFNWYV DGVEVHNAKT KPREEQYNST YRVVSVLTVL HQDWLNGKEY |
| 201 | KCKVSNKALP APIEKTISKA KGQPREPQVC TLPPSREEMT KNQVSLSCAV |
| 251 | KGFYPSDIAV EWESNGQPEN NYKTTPPVLD SDGSFFLVSK LTVDKSRWQQ |
| 301 | GNVFSCSVMH EALHNHYTQK SLSLSPGK |

The ENG(26-346)-Fc and ALK1-Fc proteins of SEQ ID NO: 802 and SEQ ID NO: 804, respectively, may be co-expressed and purified from a CHO cell line, to give rise to a heteromultimer comprising ENG(26-346)-Fc:ALK1-Fc.

A third approach to promote the formation of heteromultimer complexes using asymmetric Fc fusion proteins is illustrated in the ENG-Fc and ALK1-Fc polypeptide sequences of SEQ ID NOs: 807-809 and 810-812, respectively. In this approach, the Fc domains are altered to introduce complementary hydrophobic interactions and an additional intermolecular disulfide bond as described in the second approach above. In addition, substitutions are also made in the Fc domains to alter net molecular charge, thereby facilitating purification of the desired protein complex. The ENG(26-346)-Fc fusion polypeptide and ALK1-Fc fusion polypeptide each employ the TPA leader.

The ENG(26-346)-Fc polypeptide sequence (SEQ ID NO: 807) is shown below:

| (SEQ ID NO: 807) | |
|---|---|
| 1 | MDAMKRGLCC VLLLCGAVFV SPGAETVHCD LQPVGPERDE VTYTTSQVSK |
| 51 | GCVAQAPNAI LEVHVLFLEF PTGPSQLELT LQASKQNGTW PREVLLVLSV |
| 101 | NSSVFLHLQA LGIPLHLAYN SSLVTFQEPP GVNTTELPSF PKTQILEWAA |
| 151 | ERGPITSAAE LNDPQSILLR LGQAQGSLSF CMLEASQDMG RTLEWRPRTP |
| 201 | ALVRGCHLEG VAGHKEAHIL RVLPGHSAGP RTVTVKVELS CAPGDLDAVL |
| 251 | ILQGPPYVSW LIDANHNMQI WTTGEYSFKI FPEKNIRGFK LPDTPQGLLG |
| 301 | EARMLNASIV ASFVELPLAS IVSLHASSCG GRLQTSPAPI QTTPPTGGGT |
| 351 | HTCPPCPAPE LLGGPSVFLF PPKPKDTLMI SRTPEVTCVV VDVSHEDPEV |
| 401 | KFNWYVDGVE VHNAKTKPRE EQYNSTYRVV SVLTVLHQDW LNGKEYKCKV |
| 451 | SNKALPAPIE KTISKAKGQP REPQVCTLPP SREEMTKNQV SLSCAVKGFY |
| 501 | PSDIAVEWES RGQPENNYKT TPPVLDSRGS FFLVSKLTVD KSRWQQGNVF |
| 551 | SCSVMHEALH NHYTQKSLSL SPG |

The leader sequence and linker sequence are underlined. To promote formation of the ENG(26-346)-Fc:ALK1-Fc heterodimer rather than either of the possible homodimerics, four amino acid substitutions (replacement of a tyrosine with cysteine, a threonine with serine, a leucine with alanine, and a tyrosine with valine) can be introduced into the Fc domain of the ENG fusion polypeptide as indicated by double underline above. As indicated by single underline, two additional amino acid substitutions (replacement of an asparagine and an aspartate with arginines) can also be introduced into this Fc domain to facilitate purification of the desired heterodimer on the basis of net molecular charge. The amino acid sequence of SEQ ID NO: 807 may optionally be provided with a lysine added at the C-terminus.

This ENG(26-346)-Fc fusion protein is encoded by the following nucleic acid sequence (SEQ ID NO: 808) in which the leader sequence and linker sequence are underlined:

| (SEQ ID NO: 808) | |
|---|---|
| 1 | ATGGATGCAA TGAAGAGAGG GCTCTGCTGT GTGCTGCTGC TGTGTGGAGC |
| 51 | AGTCTTCGTT TCGCCCGGCG CCGAAACAGT CCATTGTGAC CTTCAGCCTG |
| 101 | TGGGCCCCGA GAGGGACGAG GTGACATATA CCACTAGCCA GGTCTCGAAG |
| 151 | GGCTGCGTGG CTCAGGCCCC CAATGCCATC CTTGAAGTCC ATGTCCTCTT |
| 201 | CCTGGAGTTC CCAACGGGCC CGTCACAGCT GGAGCTGACT CTCCAGGCAT |
| 251 | CCAAGCAAAA TGGCACCTGG CCCCGAGAGG TGCTTCTGGT CCTCAGTGTA |
| 301 | AACAGCAGTG TCTTCCTGCA TCTCCAGGCC CTGGGAATCC CACTGCACTT |
| 351 | GGCCTACAAT TCCAGCCTGG TCACCTTCCA AGAGCCCCCG GGGGTCAACA |
| 401 | CCACAGAGCT GCCATCCTTC CCCAAGACCC AGATCCTTGA GTGGGCAGCT |
| 451 | GAGAGGGGCC CCATCACCTC TGCTGCTGAG CTGAATGACC CCCAGAGCAT |
| 501 | CCTCCTCCGA CTGGGCCAAG CCCAGGGGTC ACTGTCCTTC TGCATGCTGG |
| 551 | AAGCCAGCCA GGACATGGGC CGCACGCTCG AGTGGCGGCC GCGTACTCCA |
| 601 | GCCTTGGTCC GGGGCTGCCA CTTGGAAGGC GTGGCCGGCC ACAAGGAGGC |
| 651 | GCACATCCTG AGGGTCCTGC CGGGCCACTC GGCCGGGCCC CGGACGGTGA |
| 701 | CGGTGAAGGT GGAACTGAGC TGCGCACCCG GGGATCTCGA TGCCGTCCTC |
| 751 | ATCCTGCAGG GTCCCCCCTA CGTGTCCTGG CTCATCGACG CCAACCACAA |
| 801 | CATGCAGATC TGGACCACTG GAGAATACTC CTTCAAGATC TTTCCAGAGA |
| 851 | AAAACATTCG TGGCTTCAAG CTCCCAGACA CACCTCAAGG CCTCCTGGGG |
| 901 | GAGGCCCGGA TGCTCAATGC CAGCATTGTG GCATCCTTCG TGGAGCTACC |
| 951 | GCTGGCCAGC ATTGTCTCAC TTCATGCCTC CAGCTGCGGT GGTAGGCTGC |
| 1001 | AGACCTCACC CGCACCGATC CAGACCACTC CTCCCACCGG TGGTGGAACT |
| 1051 | CACACATGCC CACCGTGCCC AGCACCTGAA CTCCTGGGGG GACCGTCAGT |
| 1101 | CTTCCTCTTC CCCCCAAAAC CCAAGGACAC CCTCATGATC TCCCGGACCC |
| 1151 | CTGAGGTCAC ATGCGTGGTG GTGGACGTGA GCCACGAAGA CCCTGAGGTC |
| 1201 | AAGTTCAACT GGTACGTGGA CGGCGTGGAG GTGCATAATG CCAAGACAAA |
| 1251 | GCCGCGGGAG GAGCAGTACA ACAGCACGTA CCGTGTGGTC AGCGTCCTCA |
| 1301 | CCGTCCTGCA CCAGGACTGG CTGAATGGCA AGGAGTACAA GTGCAAGGTC |
| 1351 | TCCAACAAAG CCCTCCCAGC CCCCATCGAG AAAACCATCT CCAAAGCCAA |
| 1401 | AGGGCAGCCC CGAGAACCAC AGGTGTGCAC CCTGCCCCCA TCCCGGGAGG |
| 1451 | AGATGACCAA GAACCAGGTC AGCCTGTCCT GCGCCGTCAA AGGCTTCTAT |
| 1501 | CCCAGCGACA TCGCCGTGGA GTGGGAGAGC CGCGGGCAGC CGGAGAACAA |
| 1551 | CTACAAGACC ACGCCTCCCG TGCTGGACTC CCGCGGCTCC TTCTTCCTCG |
| 1601 | TGAGCAAGCT CACCGTGGAC AAGAGCAGGT GGCAGCAGGG GAACGTCTTC |
| 1651 | TCATGCTCCG TGATGCATGA GGCTCTGCAC AACCACTACA CGCAGAAGAG |
| 1701 | CCTCTCCCTG TCTCCGGGT |

A processed ENG(26-346)-Fc fusion polypeptide (SEQ ID NO: 809) is as follows and may optionally be provided with a lysine added at the C-terminus.

| (SEQ ID NO: 809) | |
|---|---|
| 1 | ETVHCDLQPV GPERDEVTYT TSQVSKGCVA QAPNAILEVH VLFLEFPTGP |
| 51 | SQLELTLQAS KQNGTWPREV LLVLSVNSSV FLHLQALGIP LHLAYNSSLV |
| 101 | TFQEPPGVNT TELPSFPKTQ ILEWAAERGP ITSAAELNDP QSILLRLGQA |
| 151 | QGSLSFCMLE ASQDMGRTLE WRPRTPALVR GCHLEGVAGH KEAHILRVLP |
| 201 | GHSAGPRTVT VKVELSCAPG DLDAVLILQG PPYVSWLIDA NHNMQIWTTG |
| 251 | EYSFKIFPEK NIRGFKLPDT PQGLLGEARM LNASIVASFV ELPLASIVSL |
| 301 | HASSCGGRLQ TSPAPIQTTP PTGGGTHTCP PCPAPELLGG PSVFLFPPKP |
| 351 | KDTLMISRTP EVTCVVVDVS HEDPEVKFNW YVDGVEVHNA KTKPREEQYN |
| 401 | STYRVVSVLT VLHQDWLNGK EYKCKVSNKA LPAPIEKTIS KAKGQPREPQ |
| 451 | VCTLPPSREE MTKNQVSLSC AVKGFYPSDI AVEWESRGQP ENNYKTTPPV |
| 501 | LDSRGSFFLV SKLTVDKSRW QQGNVFSCSV MHEALHNHYT QKSLSLSPG |

The complementary form of ALK1-Fc fusion polypeptide (SEQ ID NO: 810) is as follows:

| (SEQ ID NO: 810) | |
|---|---|
| 1 | MDAMKRGLCC VLLLCGAVFV SPGADPVKPS RGPLVTCTCE SPHCKGPTCR |
| 51 | GAWCTVVLVR EEGRHPQEHR GCGNLHRELC RGRPTEFVNH YCCDSHLCNH |
| 101 | NVSLVLEATQ PPSEQPGTDG QLATGGGTHT CPPCPAPELL GGPSVFLFPP |
| 151 | KPKDTLMISR TPEVTCVVVD VSHEDPEVKF NWYVDGVEVH NAKTKPREEQ |
| 201 | YNSTYRVVSV LTVLHQDWLN GKEYKCKVSN KALPAPIEKT ISKAKGQPRE |
| 251 | PQVYTLPPCR EEMTENQVSL WCLVKGFYPS DIAVEWESNG QPENNYKTTP |
| 301 | PVLDSDGSFF LYSKLTVDKS RWQQGNVFSC SVMHEALHNH YTQDSLSLSP |
| 351 | G |

The leader sequence and linker sequence are underlined. To guide heterodimer formation with the ENG(26-346)-Fc fusion polypeptide of SEQ ID NOs: 807 and 809 above, two amino acid substitutions (replacing a serine with cysteine and a threonine with tryptophan) can be introduced into the Fc domain of the ALK1-Fc fusion polypeptide as indicated by double underline above. As indicated by single underline, two additional amino acid substitutions (replacement of lysines with glutamate and aspartate) can also be introduced into this Fc domain to facilitate purification of the desired heterodimer on the basis of net molecular charge. The amino acid sequence of SEQ ID NO: 810 may optionally be provided with a lysine added at the C-terminus.

This ALK1-Fc fusion protein is encoded by the following nucleic acid (SEQ ID NO: 811) in which the leader sequence and linker sequence are underlined:

| (SEQ ID NO: 811) | |
|---|---|
| 1 | ATGGATGCAA TGAAGAGAGG GCTCTGCTGT GTGCTGCTGC TGTGTGGAGC |
| 51 | AGTCTTCGTT TCGCCCGGCG CCGACCCTGT GAAGCCGTCT CGGGGCCCGC |
| 101 | TGGTGACCTG CACGTGTGAG AGCCCACATT GCAAGGGGCC TACCTGCCGG |
| 151 | GGGGCCTGGT GCACAGTAGT GCTGGTGCGG GAGGAGGGGA GGCACCCCCA |
| 201 | GGAACATCGG GGCTGCGGGA ACTTGCACAG GGAGCTCTGC AGGGGCCGCC |
| 251 | CCACCGAGTT CGTCAACCAC TACTGCTGCG ACAGCCACCT CTGCAACCAC |
| 301 | AACGTGTCCC TGGTGCTGGA GGCCACCCAA CCTCCTTCGG AGCAGCCGGG |
| 351 | AACAGATGGC CAGCTGGCCA CCGGTGGTGG AACTCACACA TGCCCACCGT |
| 401 | GCCCAGCACC TGAACTCCTG GGGGGACCGT CAGTCTTCCT CTTCCCCCCA |
| 451 | AAACCCAAGG ACACCCTCAT GATCTCCCGG ACCCCTGAGG TCACATGCGT |
| 501 | GGTGGTGGAC GTGAGCCACG AAGACCCTGA GGTCAAGTTC AACTGGTACG |
| 551 | TGGACGGCGT GGAGGTGCAT AATGCCAAGA CAAAGCCGCG GGAGGAGCAG |
| 601 | TACAACAGCA CGTACCGTGT GGTCAGCGTC CTCACCGTCC TGCACCAGGA |
| 651 | CTGGCTGAAT GGCAAGGAGT ACAAGTGCAA GGTCTCCAAC AAAGCCCTCC |
| 701 | CAGCCCCCAT CGAGAAAACC ATCTCCAAAG CCAAAGGGCA GCCCCGAGAA |
| 751 | CCACAGGTGT ACACCCTGCC CCCATGCCGG GAGGAGATGA CCGAGAACCA |
| 801 | GGTCAGCCTG TGGTGCCTGG TCAAAGGCTT CTATCCCAGC GACATCGCCG |
| 851 | TGGAGTGGGA GAGCAATGGG CAGCCGGAGA ACAACTACAA GACCACGCCT |
| 901 | CCCGTGCTGG ACTCCGACGG CTCCTTCTTC CTCTATAGCA AGCTCACCGT |
| 951 | GGACAAGAGC AGGTGGCAGC AGGGGAACGT CTTCTCATGC TCCGTGATGC |
| 1001 | ATGAGGCTCT GCACAACCAC TACACGCAGG ACAGCCTCTC CCTGTCTCCG |
| 1051 | GGT |

The mature ALK1-Fc fusion protein sequence (SEQ ID NO: 812) is as follows and may optionally be provided with a lysine added at the C-terminus.

| (SEQ ID NO: 812) | |
|---|---|
| 1 | DPVKPSRGPL VTCTCESPHC KGPTCRGAWC TVVLVREEGR HPQEHRGCGN |
| 51 | LHRELCRGRP TEFVNHYCCD SHLCNHNVSL VLEATQPPSE QPGTDGQLAT |
| 101 | GGGTHTCPPC PAPELLGGPS VFLFPPKPKD TLMISRTPEV TCVVVDVSHE |
| 151 | DPEVKFNWYV DGVEVHNAKT KPREEQYNST YRVVSVLTVL HQDWLNGKEY |
| 201 | KCKVSNKALP APIEKTISKA KGQPREPQVY TLPPCREEMT ENQVSLWCLV |
| 251 | KGFYPSDIAV EWESNGQPEN NYKTTPPVLD SDGSFFLYSK LTVDKSRWQQ |
| 301 | GNVFSCSVMH EALHNHYTQD SLSLSPG |

The ENG(26-346)-Fc and ALK1-Fc proteins of SEQ ID NO: 809 and SEQ ID NO: 812, respectively, may be co-expressed and purified from a CHO cell line, to give rise to a heteromeric complex comprising ENG(26-346)-Fc:ALK1-Fc.

Purification of various ENG-Fc:ALK1-Fc complexes could be achieved by a series of column chromatography steps, including, for example, three or more of the following, in any order: protein A chromatography, Q sepharose chromatography, phenylsepharose chromatography, size exclusion chromatography, cation exchange chromatography, epitope-based affinity chromatography (e.g., with an antibody or functionally equivalent ligand directed against an epitope on ENG or ALK1), and multimodal chromatography (e.g., with resin containing both electrostatic and hydrophobic ligands). The purification could be completed with viral filtration and buffer exchange.

### Example 2. Ligand Binding Profile of an ENG-Fc:ALK1-Fc Heterodimer

In a preliminary screen of 20 different ligands, only BMP9 and BMP10 exhibited binding to an ENG(26-346)-Fc:ALK1-Fc heterodimeric complex. A Biacore™-based binding assay was then used to compare ligand binding properties of ENG(26-346)-Fc:ALK1-Fc heterodimer with those of ENG(26-346)-Fc:Fc monomeric, ALK1-Fc:Fc monomeric complex, and ALK1-Fc:ALK1-Fc homodimer. These protein complexes were independently captured in the system using an anti-Fc antibody. Ligands were injected and allowed to flow over the captured receptor protein. Results are summarized in the table below, in which ligand off-rates (k_{d}) most indicative of effective ligand traps are denoted by bold text.

Ligand binding profile of ENG(26-346)-Fc:ALK1-Fc heterodimer compared to
ENG(26-346)-Fc monomer, ALK1-Fc monomer, and ALK1-Fc homodimer

| | Ligand | | | | | |
|---|---|---|---|---|---|---|
| | BMP9 | | | BMP10 | | |
| | kₐ | k_{d} | K_{D} | kₐ | k_{d} | K_{D} |
| Protein | (1/Ms) | (1/s) | (pM) | (1/Ms) | (1/s) | (pM) |
| ENG(26-346)-Fc:Fc | Transient binding | | >36000 | No binding | | |
| ALK1-Fc:Fc | 5.1 × 10⁶ | 1.1 × 10⁻³ | 210 | 7.5 × 10⁶ | 5.1 × 10⁻⁴ | 69 |
| ALK1-Fc:ALK1-Fc | 7.9 × 10⁶ | 1.3 × 10⁻⁴ | 16 | 4.1 × 10⁶ | 1.6 × 10⁻⁴ | 37 |
| ENG(26-346)-Fc:ALK1-Fc | 1.1 × 10⁷ | 3.9 × 10⁻⁴ | 35 | 1.4 × 10⁷ | 4.7 × 10⁻⁴ | 33 |

These binding data demonstrate that an ENG-Fc:ALK1-Fc heterodimer has useful ligand binding properties differing from those exhibited by either of the monomeric complexes. Specifically, the ENG(26-346)-Fc:ALK1-Fc heterodimer displayed enhanced binding to BMP9 and similar binding to BMP10 compared with the ALK1-Fc:Fc complex, whereas the ENG(26-346)-Fc:Fc complex showed only transient binding to BMP9 and no binding to BMP10. On the other hand, ENG(26-346)-Fc:ALK1-Fc heterodimer bound BMP9 and BMP10 with off-rate constants (k_{d}) and equilibrium dissociation constants (K_{D}) similar to those of ALK1-Fc homodimer. These results therefore demonstrate that an ENG-Fc:ALK1-Fc heterodimer has a ligand binding profile similar to that of ALK1-Fc homodimer. Accordingly, an ENG-Fc:ALK1-Fc heterodimer will be useful in therapeutic applications where it is desirable to antagonize BMP9 and BMP10.

### Example 3. Generation of an ENG-Fc:ALK2-Fc Heterodimer

A soluble ENG-Fc:ALK2-Fc heterodimer can be generated comprising a C-terminally truncated extracellular domain of human endoglin (ENG) and the extracellular domain of human ALK2, which are each fused to an Fc domain with a linker positioned between the extracellular domain and the Fc domain. The individual constructs are referred to as ENG(26-346)-Fc and ALK2-Fc fusion proteins, respectively.

Formation of heteromeric ENG(26-346)-Fc:ALK2-Fc may be guided by approaches similar to those described in Example 1.

In a first approach, the polypeptide sequence of the ENG(26-346)-Fc fusion protein and a nucleic acid sequence encoding it are provided in Example 1 as SEQ ID NOs: 101-103.

The complementary ALK2-Fc fusion protein employs the TPA leader and is as follows (SEQ ID NO: 107):

| (SEQ ID NO: 107) | |
|---|---|
| 1 | MDAMKRGLCC VLLLCGAVFV SPGAMEDEKP KVNPKLYMCV CEGLSCGNED |
| 51 | HCEGQQCFSS LSINDGFHVY QKGCFQVYEQ GKMTCKTPPS PGQAVECCQG |
| 101 | DWCNRNITAQ LPTKGKSFPG TQNFHLETGG GTHTCPPCPA PELLGGPSVF |
| 151 | LFPPKPKDTL MISRTPEVTC VVVDVSHEDP EVKFNWYVDG VEVHNAKTKP |
| 201 | REEQYNSTYR VVSVLTVLHQ DWLNGKEYKC KVSNKALPAP IEKTISKAKG |
| 251 | QPREPQVYTL PPSREEMTKN QVSLTCLVKG FYPSDIAVEW ESNGQPENNY |
| 301 | DTTPPVLDSD GSFFLYSDLT VDKSRWQQGN VFSCSVMHEA LHNHYTQKSL |
| 351 | SLSPG |

The signal sequence and linker sequence are underlined. To promote formation of the ENG(26-346)-Fc:ALK2-Fc heterodimer rather than either of the possible homodimeric complexes, two amino acid substitutions (replacing lysines with aspartic acids) can be introduced into the Fc domain of the fusion protein as indicated by double underline above. The amino acid sequence of SEQ ID NO: 107 may optionally be provided with a lysine added at the C-terminus.

This ALK2-Fc fusion protein is encoded by the following nucleic acid (SEQ ID NO: 108):

| (SEQ ID NO: 108) | |
|---|---|
| 1 | ATGGATGCAA TGAAGAGAGG GCTCTGCTGT GTGCTGCTGC TGTGTGGAGC |
| 51 | AGTCTTCGTT TCGCCCGGCG CCATGGAAGA TGAGAAGCCC AAGGTCAACC |
| 101 | CCAAACTCTA CATGTGTGTG TGTGAAGGTC TCTCCTGCGG TAATGAGGAC |
| 151 | CACTGTGAAG GCCAGCAGTG CTTTTCCTCA CTGAGCATCA ACGATGGCTT |
| 201 | CCACGTCTAC CAGAAAGGCT GCTTCCAGGT TTATGAGCAG GGAAAGATGA |
| 251 | CCTGTAAGAC CCCGCCGTCC CCTGGCCAAG CTGTGGAGTG CTGCCAAGGG |
| 301 | GACTGGTGTA ACAGGAACAT CACGGCCCAG CTGCCCACTA AAGGAAAATC |
| 351 | CTTCCCTGGA ACACAGAATT TCCACTTGGA GACCGGTGGT GGAACTCACA |
| 401 | CATGCCCACC GTGCCCAGCA CCTGAACTCC TGGGGGGACC GTCAGTCTTC |
| 451 | CTCTTCCCCC CAAAACCCAA GGACACCCTC ATGATCTCCC GGACCCCTGA |
| 501 | GGTCACATGC GTGGTGGTGG ACGTGAGCCA CGAAGACCCT GAGGTCAAGT |
| 551 | TCAACTGGTA CGTGGACGGC GTGGAGGTGC ATAATGCCAA GACAAAGCCG |
| 601 | CGGGAGGAGC AGTACAACAG CACGTACCGT GTGGTCAGCG TCCTCACCGT |
| 651 | CCTGCACCAG GACTGGCTGA ATGGCAAGGA GTACAAGTGC AAGGTCTCCA |
| 701 | ACAAAGCCCT CCCAGCCCCC ATCGAGAAAA CCATCTCCAA AGCCAAAGGG |
| 751 | CAGCCCCGAG AACCACAGGT GTACACCCTG CCCCCATCCC GGGAGGAGAT |
| 801 | GACCAAGAAC CAGGTCAGCC TGACCTGCCT GGTCAAAGGC TTCTATCCCA |
| 851 | GCGACATCGC CGTGGAGTGG GAGAGCAATG GGCAGCCGGA GAACAACTAC |
| 901 | GACACCACGC CTCCCGTGCT GGACTCCGAC GGCTCCTTCT TCCTCTATAG |
| 951 | CGACCTCACC GTGGACAAGA GCAGGTGGCA GCAGGGGAAC GTCTTCTCAT |
| 1001 | GCTCCGTGAT GCATGAGGCT CTGCACAACC ACTACACGCA GAAGAGCCTC |
| 1051 | TCCCTGTCTC CGGGT |

A processed ALK2-Fc fusion protein sequence (SEQ ID NO: 109) is as follows and may optionally be provided with a lysine added at the C-terminus.

| (SEQ ID NO: 109) | |
|---|---|
| 1 | MEDEKPKVNP KLYMCVCEGL SCGNEDHCEG QQCFSSLSIN DGFHVYQKGC |
| 51 | FQVYEQGKMT CKTPPSPGQA VECCQGDWCN RNITAQLPTK GKSFPGTQNF |
| 101 | HLETGGGTHT CPPCPAPELL GGPSVFLFPP KPKDTLMISR TPEVTCVVVD |
| 151 | VSHEDPEVKF NWYVDGVEVH NAKTKPREEQ YNSTYRVVSV LTVLHQDWLN |
| 201 | GKEYKCKVSN KALPAPIEKT ISKAKGQPRE PQVYTLPPSR EEMTKNQVSL |
| 251 | TCLVKGFYPS DIAVEWESNG QPENNYDTTP PVLDSDGSFF LYSDLTVDKS |
| 301 | RWQQGNVFSC SVMHEALHNH YTQKSLSLSP G |

The ENG(26-346)-Fc and ALK2-Fc fusion proteins of SEQ ID NO: 103 and SEQ ID NO: 109, respectively, may be co-expressed and purified from a CHO cell line to give rise to a heteromeric complex comprising ENG(26-346)-Fc:ALK2-Fc.

In another approach to promoting the formation of heteromultimers using asymmetric Fc fusion proteins, illustrated in the ENG(26-346)-Fc and ALK2-Fc polypeptide sequences of SEQ ID NOs: 801-802 and 805-806, respectively, the Fc domains are altered to introduce complementary hydrophobic interactions and an additional intermolecular disulfide bond. The ENG(26-346)-Fc fusion polypeptide sequences are discussed in Example 1.

The complementary form of ALK2-Fc fusion polypeptide (SEQ ID NO: 805) is as follows:

| (SEQ ID NO: 805) | |
|---|---|
| 1 | MDAMKRGLCC VLLLCGAVFV SPGAMEDEKP KVNPKLYMCV CEGLSCGNED |
| 51 | HCEGQQCFSS LSINDGFHVY QKGCFQVYEQ GKMTCKTPPS PGQAVECCQG |
| 101 | DWCNRNITAQ LPTKGKSFPG TQNFHLETGG GTHTCPPCPA PELLGGPSVF |
| 151 | LFPPKPKDTL MISRTPEVTC VVVDVSHEDP EVKFNWYVDG VEVHNAKTKP |
| 201 | REEQYNSTYR VVSVLTVLHQ DWLNGKEYKC KVSNKALPAP IEKTISKAKG |
| 251 | QPREPQVCTL PPSREEMTKN QVSLSCAVKG FYPSDIAVEW ESNGQPENNY |
| 301 | KTTPPVLDSD GSFFLVSKLT VDKSRWQQGN VFSCSVMHEA LHNHYTQKSL |
| 351 | SLSPGK |

The leader sequence and linker sequence are underlined. To guide heterodimer formation with the ENG(26-346)-Fc fusion polypeptide of SEQ ID NOs 801 and 802 above, four amino acid substitutions can be introduced into the Fc domain of the ALK2 fusion polypeptide as indicated by double underline above. The amino acid sequence of SEQ ID NO: 805 may optionally be provided with the lysine removed from the C-terminus.

A processed ALK2-Fc fusion protein sequence (SEQ ID NO: 806) is as follows and may optionally be provided with the lysine removed from the C-terminus.

| (SEQ ID NO: 806) | |
|---|---|
| 1 | MEDEKPKVNP KLYMCVCEGL SCGNEDHCEG QQCFSSLSIN DGFHVYQKGC |
| 51 | FQVYEQGKMT CKTPPSPGQA VECCQGDWCN RNITAQLPTK GKSFPGTQNF |
| 101 | HLETGGGTHT CPPCPAPELL GGPSVFLFPP KPKDTLMISR TPEVTCVVVD |
| 151 | VSHEDPEVKF NWYVDGVEVH NAKTKPREEQ YNSTYRVVSV LTVLHQDWLN |
| 201 | GKEYKCKVSN KALPAPIEKT ISKAKGQPRE PQVCTLPPSR EEMTKNQVSL |
| 251 | SCAVKGFYPS DIAVEWESNG QPENNYKTTP PVLDSDGSFF LVSKLTVDKS |
| 301 | RWQQGNVFSC SVMHEALHNH YTQKSLSLSP GK |

The ENG(26-346)-Fc and ALK2-Fc proteins of SEQ ID NO: 802 and SEQ ID NO: 806, respectively, may be co-expressed and purified from a CHO cell line, to give rise to a heteromeric complex comprising ENG(26-346)-Fc:ALK2-Fc.

Purification of various ENG(26-346)-Fc:ALK2-Fc complexes could be achieved by a series of column chromatography steps, including, for example, three or more of the following, in any order: protein A chromatography, Q sepharose chromatography, phenylsepharose chromatography, size exclusion chromatography, cation exchange chromatography, and epitope-based affinity chromatography (e.g., with an antibody or functionally equivalent ligand directed against an epitope on ENG or ALK2), and multimodal chromatography (e.g., with resin containing both electrostatic and hydrophobic ligands). The purification could be completed with viral filtration and buffer exchange.

### Example 4. Exemplary Co-Receptor Polypeptide Elements of Heteromeric Fusion Proteins

Additional heteromeric fusion proteins can be generated which comprise at least one co-receptor polypeptide and at least one type I receptor polypeptide or one type II receptor polypeptide. Exemplary co-receptor polypeptides for such heteromeric fusion protein complexes are provided herein as soluble fragments of TGFβ superfamily co-receptors whose NCBI Reference Sequence numbers are indicated in FIGS. 11A and 11B. An individual co-receptor isoform can give rise to multiple exemplary polypeptides of different lengths as shown in FIGS. 11A and 11B.

A soluble heteromultimer can be generated comprising a soluble human co-receptor polypeptide, or fragment thereof, and an extracellular domain, or fragment thereof, of a human type I or type II receptor, which are each fused to an Fc domain with optionally a linker positioned between the non-Fc polypeptide and the Fc domain to yield a co-receptor-Fc:receptor-Fc protein complex. A methodology for promoting formation of desirable heteromultimers, as opposed to unwanted homomultimers, is to introduce alterations in the amino acid sequence of the Fc domains to guide the formation of asymmetric heteromultimers. Many different approaches to making asymmetric interaction pairs using Fc domains are described in this disclosure.

1. A recombinant heteromultimer comprising a TGF-beta superfamily co-receptor polypeptide and a TGF-beta superfamily type I receptor polypeptide, wherein the TGF-beta superfamily co-receptor polypeptide is endoglin, and wherein the TGF-beta superfamily type I receptor is ALK1.

2-213. (canceled)

214. The recombinant heteromultimer of claim 1, wherein the ALK1 polypeptide is selected from the group consisting of:
a) a polypeptide that is at least 70%, 75%, 80%, 85%, 90%, 91%, 92%, 93%, 94%, 95%, 96%, 97%, 98%, 99%, or 100% identical to the amino acid sequence of SEQ ID NOs: 14 or 15;
b) a polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 22-34 (e.g., amino acid residues 22, 23, 24, 25, 26, 27, 28, 29, 30, 31, 32, 33, or 34) of SEQ ID NO: 14, and ends at any one of amino acids 95-118 (e.g., amino acid residues 95, 96, 97, 98, 99, 100, 101, 102, 103, 104, 105, 106, 107, 108, 109, 110, 111, 112, 113, 114, 115, 116, 117, 118, or 119) of SEQ ID NO: 14;
c) a polypeptide that is at least 70%, 75%, 80%, 85%, 90%, 91%, 92%, 93%, 94%, 95%, 96%, 97%, 98%, 99%, or 100% identical to amino acids of 22-118 of SEQ ID NO: 14; and
d) a polypeptide that is at least 70%, 75%, 80%, 85%, 90%, 91%, 92%, 93%, 94%, 95%, 96%, 97%, 98%, 99%, or 100% identical to amino acids of 34-95 of SEQ ID NO: 14.

215-226. (canceled)

227. The recombinant heteromultimer of claim 1, wherein the endoglin polypeptide is selected from the group consisting of:
a) a polypeptide that is at least 70%, 75%, 80%, 85%, 90%, 91%, 92%, 93%, 94%, 95%, 96%, 97%, 98%, 99%, or 100% identical to the amino acid sequence of SEQ ID NOs: 501, 502, 505, 506, 509, 510, or 593;
b) polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 26-30 (e.g., amino acid residues 26, 27, 28, 29, or 30) of SEQ ID NO: 501, and ends at any one of amino acids 330-346 (e.g., amino acid residues 330, 331, 332, 333, 334, 335, 336, 337, 338, 339, 340, 341, 342, 343, 344, 345, or 346) of SEQ ID NO: 501;
c) a polypeptide that is at least 70%, 75%, 80%, 85%, 90%, 91%, 92%, 93%, 94%, 95%, 96%, 97%, 98%, 99%, or 100% identical to amino acids of 30-330 of SEQ ID NO: 501;
d) a polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 26-30 (e.g., amino acid residues 26, 27, 28, 29, or 30) of SEQ ID NO: 505, and ends at any one of amino acids 330-346 (e.g., amino acid residues 330, 331, 332, 333, 334, 335, 336, 337, 338, 339, 340, 341, 342, 343, 344, 345, or 346) of SEQ ID NO: 505;
e) a polypeptide that is at least 70%, 75%, 80%, 85%, 90%, 91%, 92%, 93%, 94%, 95%, 96%, 97%, 98%, 99%, or 100% identical to amino acids of 30-330 of SEQ ID NO: 505;
f) a polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 1-25 (e.g., amino acid residues 1, 2, 3, 4, 5, 6, 7, 8, 9, 10, 11, 12, 13, 14, 15, 16, 17, 18, 19, 20, 21, 22, 23, 24, or 25) of SEQ ID NO: 509, and ends at any one of amino acids 148-164 (e.g., amino acid residues 148, 149, 150, 151, 152, 153, 154, 155, 156, 157, 158, 159, 160, 161, 162, 163, or 164) of SEQ ID NO: 509;
g) a polypeptide that is at least 70%, 75%, 80%, 85%, 90%, 91%, 92%, 93%, 94%, 95%, 96%, 97%, 98%, 99%, or 100% identical to amino acids of 25-148 of SEQ ID NO: 509;
h) a polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 26-30 (e.g., amino acid residues 26, 27, 28, 29, or 30) of SEQ ID NO: 501, and ends at any one of amino acids 582-586 (e.g., amino acid residues 582, 583, 584, 585, or 586) of SEQ ID NO: 501;
i) a polypeptide that is at least 70%, 75%, 80%, 85%, 90%, 91%, 92%, 93%, 94%, 95%, 96%, 97%, 98%, 99%, or 100% identical to amino acids of 26-586 of SEQ ID NO: 501;
j) a polypeptide that is at least 70%, 75%, 80%, 85%, 90%, 91%, 92%, 93%, 94%, 95%, 96%, 97%, 98%, 99%, or 100% identical to amino acids of 30-582 of SEQ ID NO: 501;
k) a polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 26-30 (e.g., amino acid residues 26, 27, 28, 29, or 30) of SEQ ID NO: 505, and ends at any one of amino acids 582-586 (e.g., amino acid residues 582, 583, 584, 585, or 586) of SEQ ID NO: 505;
l) a polypeptide that is at least 70%, 75%, 80%, 85%, 90%, 91%, 92%, 93%, 94%, 95%, 96%, 97%, 98%, 99%, or 100% identical to amino acids of 26-586 of SEQ ID NO: 505;
m) a polypeptide that is at least 70%, 75%, 80%, 85%, 90%, 91%, 92%, 93%, 94%, 95%, 96%, 97%, 98%, 99%, or 100% identical to amino acids of 30-582 of SEQ ID NO: 505;
n) a polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 1-25 (e.g., amino acid residues 1, 2, 3, 4, 5, 6, 7, 8, 9, 10, 11, 12, 13, 14, 15, 16, 17, 18, 19, 20, 21, 22, 23, 24, or 25) of SEQ ID NO: 509, and ends at any one of amino acids 400-404 (e.g., amino acid residues 401, 402, 403, or 404) of SEQ ID NO: 509;
o) a polypeptide that is at least 70%, 75%, 80%, 85%, 90%, 91%, 92%, 93%, 94%, 95%, 96%, 97%, 98%, 99%, or 100% identical to amino acids of 1-404 of SEQ ID NO: 509; and
p) a polypeptide that is at least 70%, 75%, 80%, 85%, 90%, 91%, 92%, 93%, 94%, 95%, 96%, 97%, 98%, 99%, or 100% identical to amino acids of 25-400 of SEQ ID NO: 509.

228-238. (canceled)

239. The recombinant heteromultimer of claim 1, wherein the TGF-beta superfamily type I receptor polypeptide is a fusion protein further comprising a heterologous polypeptide domain, and wherein the heterologous polypeptide domain is a first or second member of an interaction pair.

240. (canceled)

241. The recombinant heteromultimer of claim 1, wherein the TGF-beta superfamily co-receptor polypeptide is a fusion protein further comprising a heterologous polypeptide domain, and wherein the heterologous polypeptide domain is a first or second member of an interaction pair.

242. The recombinant heteromultimer of claim 1, wherein the heterologous polypeptide domain comprises a constant region from an IgG heavy chain.

243. The recombinant heteromultimer of claim 242, wherein the constant region from an IgG heavy chain is an immunoglobulin Fc domain.

244. (canceled)

245. The recombinant heteromultimer of claim 242, wherein the constant region from an IgG heavy chain comprises one or more amino acid mutations (e.g., amino acid additions, deletions, or substitutions) that promote heteromultimer formation, and/or inhibit homomultimer formation.

246-252. (canceled)

253. The recombinant heteromultimer of claim 239, wherein the fusion protein comprises a linker domain positioned between the TGF-beta superfamily receptor polypeptide (e.g., TGF-beta superfamily type I receptor polypeptide, and/or TGF-beta superfamily co-receptor polypeptide) and the heterologous polypeptide domain.

254. (canceled)

255. The recombinant heteromultimer of claim 1, wherein the TGF-beta superfamily type I receptor polypeptide and/or TGF-beta superfamily co-receptor polypeptide comprises one or more amino acid modifications selected from the group consisting of: a glycosylated amino acid, a PEGylated amino acid, a farnesylated amino acid, an acetylated amino acid, a biotinylated amino acid, and an amino acid conjugated to a lipid moiety.

256. The recombinant heteromultimer of claim 1, wherein the TGF-beta superfamily type I receptor polypeptide and/or TGF-beta superfamily co-receptor polypeptide is glycosylated and has a glycosylation pattern obtainable from expression of the TGF-beta superfamily type I receptor polypeptide, TGF-beta superfamily type II receptor polypeptide, and/or TGF-beta superfamily co-receptor polypeptide in a CHO cell.

257-258. (canceled)

259. The recombinant heteromultimer of claim 1, wherein the recombinant heteromultimer binds to and/or inhibits BMP9 and/or BMP10.

260-264. (canceled)

265. The recombinant heteromultimer of claim 1, wherein the heteromultimer is an endoglin-Fc:ALK1-Fc heterodimer.

266. A pharmaceutical preparation comprising the recombinant heteromultimer of claim 1 and a pharmaceutically acceptable carrier.

267. The pharmaceutical preparation of claim 266, wherein the pharmaceutical preparation comprises less than about 10%, 9%, 8%, 7%, 6%, 5%, 4%, 3%, 2%, or less than about 1% TGF-beta type 1 receptor homomultimers and/or TGF-beta co-receptor homomultimers.

268-322. (canceled)

323. The recombinant heteromultimer of claim 1, wherein the endoglin:ALK1 heteromultimer comprises a polypeptide that is at least 90% identical to a polypeptide that begins at any one of amino acids of 22-34 of SEQ ID NO: 14 and ends at any one of amino acids 95-118 of SEQ ID NO: 14.

324. The recombinant heteromultimer of claim 1, wherein the endoglin:ALK1 heteromultimer comprises a polypeptide that is at least 90% identical to a polypeptide that begins at any one of amino acids of 26-30 of SEQ ID NO: 501, and ends at any one of amino acids 330-346 of SEQ ID NO: 501.

325. The recombinant heteromultimer of claim 1, wherein the endoglin:ALK1 heteromultimer comprises a polypeptide that is at least 90% identical to a polypeptide that begins at any one of amino acids of 22-34 of SEQ ID NO: 14 and ends at any one of amino acids 95-118 of SEQ ID NO: 14; and wherein the endoglin:ALK1 heteromultimer comprises a polypeptide that is at least 90% identical to a polypeptide that begins at any one of amino acids of 26-30 of SEQ ID NO: 501, and ends at any one of amino acids 330-346 of SEQ ID NO: 501.

326. The recombinant heteromultimer of claim 1, wherein the endoglin:ALK1 heteromultimer comprises a polypeptide that is at least 95% identical to a polypeptide that begins at any one of amino acids of 22-34 of SEQ ID NO: 14 and ends at any one of amino acids 95-118 of SEQ ID NO: 14; and wherein the endoglin:ALK1 heteromultimer comprises a polypeptide that is at least 95% identical to a polypeptide that begins at any one of amino acids of 26-30 of SEQ ID NO: 501, and ends at any one of amino acids 330-346 of SEQ ID NO: 501.

327. The recombinant heteromultimer of claim 1, wherein the endoglin:ALK1 heteromultimer comprises a polypeptide that begins at any one of amino acids of 22-34 of SEQ ID NO: 14 and ends at any one of amino acids 95-118 of SEQ ID NO: 14; and wherein the endoglin:ALK1 heteromultimer comprises a polypeptide that begins at any one of amino acids of 26-30 of SEQ ID NO: 501, and ends at any one of amino acids 330-346 of SEQ ID NO: 501.
